(19) United States
(12) Patent Application Publication
(10) Pub. No.: US 2013/0295666 A1
(43) Pub. Date: Nov. 7, 2013

(54) CRYSTALS OF GLUCOKINASE REGULATORY PROTEIN (GKRP)

(71) Applicant: Boehringer Ingelheim International GmbH, Ingelheim am Rhein (DE)

(72) Inventors: Gisela SCHNAPP, Biberach an der Riss (DE); Adina Berg, Biberach an der Riss (DE); Stefan Kauschke, Biberach an der Riss (DE); Martin Lenter, Neu-Ulm (DE); Alexander Pautsch, Biberach an der Riss (DE); Wolfgang Rist, Mittelbiberach (DE)

(73) Assignee: Boehringer Ingelheim International GmbH, Ingelheim am Rhein (DE)

(21) Appl. No.: 13/716,332

(22) Filed: Dec. 17, 2012

(30) Foreign Application Priority Data
Dec. 22, 2011 (EP) 111 95 335.2

(51) Int. Cl. C07K 14/47

(52) U.S. Cl. CPC C07K 14/4703
USPC 435/348; 530/350; 435/320.1; 536/23.5; 435/325; 530/412

(57) ABSTRACT

The present invention pertains to crystals of glucokinase regulatory protein (GKRP) and of GKRP variants, to the molecular biology of certain GKRP variants, to processes for the crystallization of GKRP and GKRP variants, to such crystals and corresponding structural information obtained by X-ray crystallography. Such crystals and crystallographic data can be used for the identification of compounds that bind to GKRP, especially of compounds that inhibit GKRP or interfere with the interaction of GKRP with its natural interacting partner Glucokinase (GK).

### FIELD OF THE INVENTION

The present invention relates to the technical field of protein biochemistry, precisely to to structural studies of proteins. The present invention pertains to crystals of glucokinase regulatory protein (GKRP) and of GKRP variants, to the molecular biology of certain GKRP variants, to processes for the crystallization of GKRP and GKRP variants, to such crystals and corresponding structural information obtained by X-ray crystallography. Such crystals and crystallographic data can be used for the identification of compounds that bind to GKRP, especially of compounds that inhibit GKRP or interfere with the interaction of GKRP with its natural interacting partner Glucokinase (GK).

### BACKGROUND OF THE INVENTION

Glucokinase (Hexokinase IV, GK) plays a major role in the regulation of blood glucose homeostasis due to its important role as the dominant glucose phosphorylating enzyme in both the liver and the pancreas, its major sites of expression. GK functions as a sensor for both the regulation of hepatic glucose metabolism (hepatic glucose uptake, hepatic glucose output) as well as for pancreatic insulin secretion. Its sigmoidal activation curve by glucose, a unique feature among the family of hexokinases, allows a fast and pronounced response in activity to fluctuations in plasma glucose levels. Using small molecule activators of GK (GKAs) in order to increase its activity is under intense investigation both preclinically as well as in clinical phases as a novel anti-diabetic principle.

In the liver, GK is regulated not only by the presence of its substrate glucose but also by a 68 kD regulatory protein, GKRP (glucokinase regulatory protein), that inhibits GK in a competitive manner with respect to glucose. In the presence of low glucose levels, GK is bound to GKRP forming an inactive complex which is predominantly localized in the nucleus. Upon replenishing glucose levels e.g. by feeding, the inactive GK-GKRP complex dissociates and a translocation of GK into the cytosol, its site of action, takes place.

In addition to the impact of glucose itself on the dissociation of the GK-GKRP complex likely via affecting GK directly, different fructose phosphates play an important role in increasing the respective probabilities of both the assembly of the inactive nuclear complex of GKRP-GK as well as its dissociation: While it could be shown that the binding of fructose-1-phosphate (F1P) to GKRP increases its affinity for GK thereby favouring the inactive complex, the binding of fructose-6-phosphate (F6P) (as well as its analogue sorbitol 6-phosphate) to GKRP on the other hand destabilizes the complex and shifts the equilibrium of total GK to the free and active form in the cytosol.

The current knowledge of the molecular details of the GK-GKRP complex is limited and originates mainly from indirect evidence, largely enzymatic experiments. While first site-directed mutagenesis efforts investigating selected amino acids on their potential involvement in fructose binding and their impact on the GK-GKRP complex formation indicated at least in part overlapping binding sites for fructose phosphates on GKRP, there is a lack of in-depth details on either the molecular structure of GKRP, the precise binding sites of these important endogenous regulators or the underlying regulatory mechanisms.

It is discussed if activators of GK could be used for the therapy of diseases of the energy metabolism, especially of type 2 diabetes. Mechanisms of activation of GK may be increasing its presence as well as the destabilizing or inhibition of the binding by GKRP. Accordingly it is desired to identify possible binding sites on GKRP and to better understand its regulation, e.g. via fructose phosphates. Such learnings could be drawn from the three-dimensional structure of GKRP which is expected to be possible via the crystallization of this protein.

Though a lot of know-how about protein crystallization has accumulated in the state of the art, every protein possesses characteristic features imposing difficulties on the crystallization. Accordingly, there is no general teaching on protein crystallization to be applied on each and every protein. In the case of GKRP the inventors were confronted especially with the problem of a well behaved protein which fulfilled all necessary quality demands for crystallization (purity, homogeneity, solubility and the like) but would nevertheless not yield to a crystal form suitable for X-ray analysis.

On the other hand a GKRP crystal was desired to understand its three-dimensional structure especially with respect to binding interfaces to other proteins like GK and/or to identify small chemical molecules that could be proposed to interfere with GKRP's in vivo interactions and biochemical activities. Such molecules could then be proposed for medical uses, as explained above.

Therefore there was a need in the state of the art to provide detailed structural data of GKRP, esp. about the active site and/or interaction sites with GK, with F1P and/or other molecules, preferably about the enzyme in total, in order to analyze its interaction with the different binding partners on a molecular basis and to provide a means for the identification of interacting molecules.

Such a GKRP should preferably be the GKRP of a mammalian organism, preferably a primate like human or closely related molecules.

Along with this need there was the necessity to define sequences of GKRP, preferably derived from a mammalian, or of variants thereof that can be used as starting points for structural analyses. Along with this need, appropriate expression systems had to be identified.

Further, there was a need to identify appropriate crystallization conditions, not only for the protein per se but also for co-crystals of GKRP or variants of GKRP in complex with one or more interacting small molecular weight chemical molecules.

### SUMMARY OF THE INVENTION

As a solution for the identified problems, the present invention provides crystals of a glucokinase regulatory protein (GKRP) comprising (i) at least 82% identity to SEQ ID NO: 2, (ii) at least 82% identity to SEQ ID NO: 4, (iii) at least 82% identity to SEQ ID NO: 6, or (iv) at least 82% identity to SEQ ID NOS: 2 and 4, to SEQ ID NOS: 4 and 6, or to SEQ ID NOS: 2 and 6. The present invention further provides crystals of a deletion mutant (truncated form of GKRP) comprising (i) at least 82% identity to positions 6 to 606 of SEQ ID NO: 2, (ii) at least 82% identity to positions 6 to 606 of SEQ ID NO: 4, (iii) at least 82% identity to positions 6 to 606 of SEQ ID NO: 6, or (iv) at least 82% identity to positions 6 to 606 of SEQ ID NOS: 2 and 4, to positions 6 to 606 of SEQ ID NOS: 4 and 6, or to positions 6 to 606 of SEQ ID NOS: 2 and 6.

The crystals of the glucokinase regulatory protein (GKRP) according to the invention may further comprise at least 85, 90, 95, 97.5, 98, 99 or 100% identity to SEQ ID NO: 2, at least 85, 90, 95, 97.5, 98, 99 or 100% identity to SEQ ID NO: 4, or at least 85, 90, 95, 97.5, 98, 99 or 100% identity to SEQ ID NO: 6.

Similarly, the crystals of the deletion mutant (truncated form) of GKRP may further comprises at least 85, 90, 95, 97.5, 98, 99 or 100% identity to positions 6 to 606 of SEQ ID NO: 2, at least 85, 90, 95, 97.5, 98, 99 or 100% identity to positions 6 to 606 of SEQ ID NO: 4, or at least 85, 90, 95, 97.5, 98, 99 or 100% identity to positions 6 to 606 of SEQ ID NO: 6.

The crystals of GKRP or deletion mutant (truncated form) of GKRP may comprise point mutations selected from 1 to 20 additional amino acids. These mutations may be added to the C- and/or N-terminus as tags. Preferably, 1 to 10 additional amino acids may be added to the C- and/or N-terminus as tags.

Where the crystals of GKRP or the deletion mutant (truncated form) of GKRP comprise one or more tags, the tags may be selected from 1 to 10 additional histidines added to the N-terminus (His-tag), optionally with a linker of 1 to 5 additional amino acids, and/or 1 to 10 additional histidines added to the C-terminus (His-tag), optionally with a linker of 1 to 5 additional amino acids.

In one embodiment, the crystals of GKRP or the deletion mutant (truncated form) of GKRP may comprise 6 additional histidines added to the C-terminus, with a linker of one aliphatic and one acidic amino acid. Preferably, the C-terminus is defined by the octapeptide LEHHHHHH or VEHHHHHH.

In another embodiment, the crystals of GKRP or the deletion mutant (truncated form) of GKRP comprises deletions of 1 to 50 amino acids from the N-terminus (N-terminal truncation) and/or from the C-terminus (C-terminal truncation) of the non-tagged GKRP or of the deletion mutant (truncated form) of GKRP. In a preferred embodiment, there is a deletion of the N-terminal 44 amino acids in the numbering according to SEQ ID NO: 2 and/or of the C-terminal 20 amino acids in the numbering according to SEQ ID NO: 2. Either the GKRP or the deletion mutant (truncated form) of GKRP may have point mutations selected from 1 to 15 deletions or substitutions of solvent exposed amino acids.

Crystals may comprise one or more of the following substitutions of solvent exposed amino acids: K164T, K165T, K170T, K171T, K326T, K327T, K450T, K451T, K567T, in the numbering according to SEQ ID NO: 2 and FIG. 9, preferably K326T and/or K327T, more preferred K326T and K327T.

In one embodiment, a crystal of GKRP or deletion mutant (truncated form) of GKRP is selected from: hGKRP (SEQ ID NO: 2), mGKRP (SEQ ID NO: 4), rGKRP (SEQ ID NO: 6), hGKRP_C-His (SEQ ID NO: 8), hGKRP_C-His_K326T/K327T (SEQ ID NO: 10), mGKRP_C-His (SEQ ID NO: 12) or rGKRP_C-His (SEQ ID NO: 14). Preferably, the crystal is hGKRP_C-His_K326T/K327T (SEQ ID NO: 10).

In another embodiment, a crystal of GKRP or deletion mutant (truncated form) of GKRP is complexed with a low molecular weight binding ligand in the active site, and preferably with a low molecular weight binding ligand selected from Fructose-1-Phosphate (F1P), Fructose-6-Phosphate (F6P), Orthophosphate (Pᵢ) or Sorbitol-6-Phosphate (S6P). Fructose-1-Phosphate (F1P) or Orthophosphate (Pᵢ) is preferred.

In a further embodiment, a crystal of GKRP or deletion mutant (truncated form) of GKRP is hGKRP_C-His_K326T/K327T (SEQ ID NO. 10), and the low molecular weight binding ligand in the active site is Fructose-1-Phosphate (F1P) or Orthophosphate (Pᵢ).

A crystal of GKRP or the deletion mutant (truncated form) of GKRP may also not be complexed with a low molecular weight binding ligand in the active site. Instead, one or more molecules of water and/or one or more of one atom cations may be complexed. Preferably one or more of water molecules, magnesium ions (Mg²⁺) and/or calcium ions (Ca²⁺) are complexed.

In one embodiment of this invention, the active site of a crystal of GKRP or the deletion mutant (truncated form) of GKRP is formed by one or more of the amino acid residues or H₂O molecules selected from Arg518, Leu515, His351, Lys514, Asn512, Ser183, Glu153, Glu348, Gly181, Ala184, Ser179, Arg259, Gly107, Val180, Thr109, Ser110, Ser258, Gly108, Ile178, a H₂O molecule complexed by Arg518 and His351, a H₂O molecule complexed by Gly153 and Ser183, a H₂O molecule complexed by Arg259 and Ser258, a H₂O molecule complexed by Thr109 or a H₂O molecule complexed by Gly107 and Ile178. Preferably, the active site of a crystal of GKRP or the deletion mutant (truncated from) of GKRP is formed by one or more of the amino acid residues selected from Lys514, Asn512, Glu153, Gly181, Ser179, Val180, Gly107, Ser110, Thr109 or Glu348, wherein all numbers refer to SEQ ID NO: 2.

A crystal of GKRP or deletion mutant (truncated form) of GKRP may also comprise a fructose-phosphate binding site at the interface between a SIS domain and a 2^{nd} α-helical domain with ubiquitin-like fold.

Preferably, the crystal according to this invention has a space group P2₁2₁2₁. Also preferred is a crystal having unit cell dimensions between 60.0 and 62.0 Å for a, between 71.5 to 73.5 Å for b, and between 136.0 and 139.0 Å for c. Preferably, the crystals has a space group of P2₁2₁2₁ and/or unit cell dimensions of a=61.0 Å, b=72.3 Å and c=136.9 Å or a space group of P2₁2₁2₁ and/or unit cell dimensions of a=60.8 Å, b=72.2 Å and c=138.0 Å.

In yet another embodiment, the crystal according to this invention has amino acids coordinated as shown in FIG. 2 or FIG. 3.

Further aspects of the invention pertain to nucleotide and amino acid sequences, vectors, host cells and related molecular biological aspects of the proteins relevant for the invention; processes for the crystallization of GKRP or GKRP variants relevant for the invention; and uses of crystals of a GKRP or GKRP variant according to the invention for the identification of low molecular chemical molecules or proteins that bind to GKRP.

In one embodiment, a polynucleotide encodes for a GKRP variant with at least one nucleotide different from SEQ ID NO: 1, 3 or 5 (other than wildtype). The polynucleotide may comprise one or more codons optimized for an expression system, preferably one or more codons optimized for the expression in an eukaryotic expression system, more preferred for the expression in mammalian or insect cells.

In another embodiment, the polynucleotide may encode for a GKRP variant selected from SEQ ID NO: 8, 10, 12, or 14, or the polynucleotide of SEQ ID NO: 7, 9, 11, 13 or 15. In a preferred embodiment, the polynucleotide may encode for a GKRP variant of SEQ ID NO: 15. The GKRP variant may have at least one amino acid difference from SEQ ID NO: 2, 4 or 6 (other than wildtype).

In yet another embodiment, the GKRP variant is selected from hGKRP_C-His (SEQ ID NO: 8), hGKRP_C-His_K326T/K327T (SEQ ID NO: 10), mGKRP_C-His (SEQ ID NO: 12) or rGKRP_C-His (SEQ ID NO: 14). In a preferred embodiment, the GKRP variant is hGKRP_C-His_K326T/K327T (SEQ ID NO: 10).

A vector comprising a polynucleotide encoding for a GKRP or GKRP variant is also part of this invention. Vector may be an expression vector. Host cells comprising a polynucleotide encoding for a GKRP or GKRP variant is also part of this invention. Specifically, host cells expressing the GKRP or GKRP variant, preferably an eukaryotic host cell, more preferred a mammalian or insect cell, mostly preferred a cell derived from Spodoptera frugiperda, are part of this invention.

Another embodiment of this invention relates to a process for the crystallization of a GKRP or GKRP variant comprising the steps of (1) purification of the protein and (2) crystallization of the purified protein.

The process may comprise, for example, in step (2), that the purified protein is complexed with a low molecular weight binding ligand in the active site, preferably with a low molecular weight binding ligand selected from Fructose-1-Phosphate (F1P), Fructose-6-Phosphate (F6P), Orthophosphate (Pᵢ) or Sorbitol-6-Phosphate (S6P), preferably Fructose-1-Phosphate (F1P) or Orthophosphate (Pᵢ).

The process may also employ using a sitting drop vapour diffusion method for step (2). Furthermore, the process step (2) may be performed between 17.5 and 22.5° C. and preceded by a preincubation of the solution of the purified GKRP or GKRP variant at 12-16 mg/ml in buffer-P2 (25 mM Hepes pH 7.4, 50 mM KCl, 1 mM MgCl₂, 2 mM DTT) supplemented with 5 mM fructose-1-phosphate (F1P) for 0.5 to 1.5 h at 3 to 5° C. According to the process of this invention, the solution of the GKRP or GKRP variant and a reservoir solution consisting of 14.4% PEG 8.000, 20% Glycerin, 0.16 M Calcium acetate and 0.08 M Cacodylate pH 6.5 are mixed in a volume ratio of 1:1 resulting in the mixture of the sitting drop, preferably by a mixture of 0.75 to 1.25 μl each.

In another embodiment, the crystals resulting from step (2) of the process are flash frozen with the mother liquor serving as cryo-protectant, preferably in a nitrogen stream below 150 K.

Also included in this invention are crystals made according to the process steps described herein.

A crystal of a GKRP or GKRP variant may also be used for the identification of a low molecular weight chemical molecule or protein that binds to GKRP. The binding low molecular chemical molecule or protein binds to the active site of GKRP and/or to the contact site of its respective Glucokinase (GK), and preferably inhibits the enzymatic activity of the GKRP and/or interferes with the interaction of the GKRP with its respective GK.

The active site of GKRP may be defined by one or more of the amino acid residues or H₂O molecules selected from Arg518, Leu515, His351, Lys514, Asn512, Ser183, Glu153, Glu348, Gly181, Ala184, Ser179, Arg259, Gly107, Val180, Thr109, Ser110, Ser258, Gly108, Ile178, a H₂O molecule complexed by Arg518 and His351, a H₂O molecule complexed by Gly153 and Ser183, a H₂O molecule complexed by Arg259 and Ser258, a H₂O molecule complexed by Thr109 and a H₂O molecule complexed by Gly107 and Ile178, preferably by one or more of the aminoacid residues selected from Lys514, Asn512, Glu153, Gly181, Ser179, Val180, Gly107, Ser110, Thr109, Glu348, wherein all numbers refer to SEQ ID NO: 2.

Additionally, the binding low molecular chemical molecule or protein may bind partially or completely to another site than the active site of GKRP but nonetheless interferes with the enzymatic activity and/or the interaction with the respective Glucokinase (GK).

The binding of the low molecular weight chemical molecule or protein may also induce a conformational change and/or stabilizes a conformation of the GKRP that negatively affects the interaction with the respective Glucokinase (GK) in comparison to the conformation of the GKRP free from the same low molecular chemical molecule or protein.

The identification may also take place by the cocrystallization with the low molecular weight chemical molecule or protein, according to a process in this invention, with the low molecular weight chemical molecule or protein instead of the otherwise complexed low molecular weight binding ligands, preferably instead of the complexed low molecular weight binding ligands. The identification may take place by soaking the crystal with a solution comprising the low molecular weight chemical molecule or protein. The identification may also take place by a computer-aided modelling program for the design of binding molecules, preferably starting from the structure of hGKRP_C-His_K326T/K327T (SEQ ID NO: 12) and the low molecular weight binding ligand in the active site selected from Fructose-1-Phosphate (F1P; FIG. 2) and Orthophosphate (Pᵢ; FIG. 3). The low molecular weight chemical molecule may also be selected from a sugar and/or phosphate containing compound.

Where a protein is used, it may be selected from antibodies. Also, a low molecular weight chemical molecule or protein may further be characterized by a biochemical assay before, after or in parallel to the use of the crystal. Finally, the biochemical assay may be characterized by the presence of glucokinase (GK; coupled assay), preferably an assay that measures the activity of glucokinase.

These and other aspects of the present invention are described herein by reference to the following figures and examples. The figures and examples serve for demonstrative purposes and do not limit the scope of the claims.

As explained below in more detail and demonstrated by the examples of this application, crystals of biochemically active GKRP variants could be prepared by the constructs and the expression systems according to the invention. The X-ray structures of two specific crystals are outlines in the figures; comparable structures of comparable crystals are now at hand and have thus enriched the state of the art.

### BRIEF DESCRIPTION OF THE DRAWINGS

FIG. 1

Diffraction quality crystals of the double mutant GKRP_{K326} (i.e. GKRP_{WT-His} K326T/K327T) in complex with Fructose-1-Phosphate, result of example 4.

FIGS. 2.1-2.142

Coordinates of hGKRP_C-His_K326T/K327T in complex with Fructose-1-Phosphate (hGKRP_C-His_K326T/K327T-F1P), result of example 4.

FIGS. 3.1-3.140

Coordinates of hGKRP_C-His_K326T/K327T in complex with Phosphate (hGKRP_C-His_K326T/K327T-P), result of example 5.

FIG. 4.1

Structure of hGKRP_C-His_K326T/K327T:

GKRP domain arrangement.

| | | |
|---|---|---|
| | 4 to 44 | N-terminus |
| SIS 1: | 45 to 284 | sugar isomerase (SIS) domain 1 |
| SIS 2: | 289 to 498 | sugar isomerase (SIS) domain 2 |
| LID: | 499 to 606 | alpha helical C-terminal domain |

FIG. 4.2

Structure of hGKRP_C-His_K326T/K327T:

Ribbon diagram of hGKRP_C-His_K326T/K327T. The individual domains are shaded as in A. F1P is shown as a sphere representation. The view is approximately down the pseudo two fold axis that relates SIS1 and SIS2.

FIG. 5.1

Fructose Phosphate Binding Site

Stick representation of the F1P binding site. Water molecules are shown as spheres, hydrogen bonds as light dotted lines. The final weighted 2|Fₒ|−|F_{c}| electron density map for F1P is shown as a mesh contoured at 1.5 σ.

FIG. 5.2

Surface plot of the F1P binding site.

FIG. 6

Schematic plot of Fructose-1-interactions in the active site of GKRP, as identified by the examples and the structure given in FIG. 2.

FIG. 7

H/D mapping of hGKRP_C-His after 1 min in D₂O-buffer.

FIG. 8

H/D mapping of hGKRP and fructose phosphate binding. Protection against H/D exchange due to ligand binding. Six regions are shown which are protected against deuterium incorporation in the presence of ligand (F6P or F1P) as compared to apo-hGKRP (D_{GKRP}: deuterium incorporation in apo-hGKRP after 30 min; D_{(hGKRP+Ligand)}: deuterium incorporation in ligand-bound hGKRP after 30 min).

FIGS. 9.1-9.2

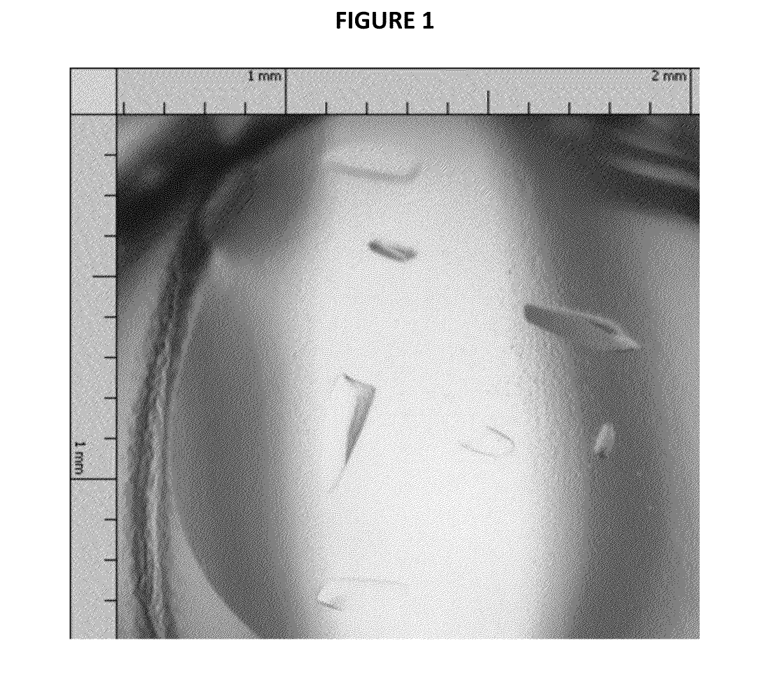

Alignment of aminoacid sequences relevant for the invention;

hGKRP: wildtype of human GKRP according to SEQ ID NO. 2

mGKRP: wildtype of mouse GKRP according to SEQ ID NO. 4

rGKRP: wildtype of rat GKRP according to SEQ ID NO. 6

Solvent exposed aminoacid positions: K164, K165, K170, K171, K326, K327, K450, K451, K567 in the numbering according to SEQ ID NO: 2 are marked in bold letters.

### SEQUENCE LISTING

### Free Text

The sequence listing enclosed with this application defines in total 18 DNA and amino acid sequences relevant for the invention.

SEQ ID NOs. 1 to 6 define wildtype sequences of GKRP derived from human (SEQ ID NOs. 1 and 2), from mouse (SEQ ID NOs. 3 and 4) and from rat (SEQ ID NOs. 5 and 6), respectively.

SEQ ID NO. 7 is an artificial DNA sequence of 1905 positions, with a coding sequence from positions 1 to 1902, characterized by this free text: human GKRP comprising C-terminal His-tag; codon optimized. SEQ ID NO. 8 is the derived amino acid sequence calculated automatically by the computer program used for the creation of the sequence listing, i.e. by Patentln version 3.3.

SEQ ID NO. 9 is an artificial DNA sequence of 1905 positions, with a coding sequence from positions 1 to 1902, characterized by this free text: human GKRP comprising C-terminal His-tag; codon optimized; variant K326T/K327T. SEQ ID NO. 10 is the derived amino acid sequence calculated by Patentln version 3.3.

SEQ ID NO. 11 is an artificial DNA sequence of 1896 positions, with a coding sequence from positions 1 to 1893, characterized by this free text: mouse GKRP comprising C-terminal His-tag. SEQ ID NO. 12 is the derived amino acid sequence calculated by Patentln version 3.3.

SEQ ID NO. 13 is an artificial DNA sequence of 1929 positions, with a coding sequence from positions 1 to 1926, characterized by this free text: rat GKRP comprising C-terminal His-tag. SEQ ID NO. 14 is the derived amino acid sequence calculated by Patentln version 3.3.

SEQ ID NO. 15 is an artificial DNA sequence of 1878 positions, with a coding sequence from positions 1 to 1875, characterized by this free text: human GKRP comprising no C-terminal His-tag; codon optimized. SEQ ID NO. 16 is the derived amino acid sequence calculated by Patentln version 3.3.

SEQ ID NO. 17 is an artificial DNA sequence of 25 positions, characterized by this free text: Primer attB1.

SEQ ID NO. 18 is an artificial DNA sequence of 24 positions, characterized by this free text: Primer attB2.

### DETAILED DESCRIPTION OF THE PRESENT INVENTION

Unless otherwise defined, all technical and scientific terms used herein have the same meaning as those commonly understood by one of ordinary skill in the art to which the invention pertains. Generally, the procedures for cell culture, infection, protein purification, molecular biology methods and the like are common methods used in the art. Such techniques can be found in reference manuals such as, for example, Sambrook et al. (2001, Molecular Cloning—A Laboratory Manual, Cold Spring Harbor Laboratory Press); Ausubel et al. (1994, Current Protocols in Molecular Biology, Wiley, New York) and Coligan et al. (1995, Current Protocols in Protein Science, Volume 1, John Wiley & Sons, Inc., New York).

Nucleotide sequences are presented herein by single strand, in the 5′ to 3′ direction, from left to right, using the one letter nucleotide symbols as commonly used in the art and in accordance with the recommendations of the IUPAC-IUB Biochemical Nomenclature Commission (Biochemistry, 1972, 11:1726-1732). The same applies mutatis mutandis to aminoacid sequences which are given from the N-terminus, on the left, to the C-terminus, on the right.

All values and concentrations presented herein are subject to inherent variations acceptable in biological science within an error of ±10%. The term “about” also refers to this acceptable variation.

A “crystal” according to the invention is a solid material whose constituent molecules are arranged in an orderly repeating pattern extending in all three spatial dimensions. The process of forming a crystalline structure from a fluid or from materials dissolved in the fluid is referred to as the crystallization process. Which crystal structure the fluid will form depends on the chemistry of the fluid, the conditions under which it is being solidified, and also on the ambient pressure.

A crystal “of a protein” according to the invention comprises molecules of the respective protein as main constituent molecules. Proteins like other chemical material can grow into protein crystals under appropriate conditions, regularly by undergoing slow precipitation, mostly from an aqueous solution. As a result, individual protein molecules align themselves in a repeating series of unit cells by adopting a consistent orientation. The forming crystalline lattice is held together by noncovalent interactions. Further molecules like water, ions or small molecule binding partners of the protein might also become integrated into the protein crystal, becoming part of the regular structure, e.g. by forming ion or hydrogen bonds to certain aminoacid sidechains in the same ordered manner. According to the invention crystallization of the relevant protein is intended to allow X-ray crystallography based on the protein crystal. This commonly known technique is used to determine the protein's three-dimensional structure via X-ray diffraction.

“Glucokinase regulatory protein (GKRP)”, also called glucokinase (hexokinase 4) regulator (GCKR) is to be understood as the glucokinase regulatory protein that interacts with and inhibits glucokinase (GK) in a competitive manner with respect to glucose. It inhibits glucokinase by forming an inactive complex with GK. The human protein is found in liver and pancreas, but not detected in muscle, brain, heart, thymus, intestine, uterus, adipose tissue, kidney, adrenal, lung or spleen. The human protein comprises 626 aminoacids and a molecular weight of about 68 kD. The structure of the protein contains two SIS (sugar isomerase) domains, as derived from sequence information. The human gene comprises 19 exons and is located on the short arm of chromosome 2 (2p23). Up to date there are four members of the GCKR family known on the aminoacid level and listed in protein databases, e.g. in UniProtKB (available via the URL http://www.uniprot.org/uniprot; inspected 20 August, 2011).

TABLE 1
Known sequenced glucokinase regulatory proteins: the
GCKR family as disclosed in the database UniProtKB

| Acces- | | | | | |
|---|---|---|---|---|---|
| sion | | Protein | Gene | | |
| number | Entry name | name | name | Organism | Length |
| Q14397 | GCKR_HUMAN | Gluco- | GCKR | Homo | 625 |
| | | kinase | | sapiens | |
| | | regulatory | | (Human) | |
| | | protein | | | |
| Q07071 | GCKR_RAT | Gluco- | Gckr | Rattus | 627 |
| | | kinase | | norvegicus | |
| | | regulatory | | (Rat) | |
| | | protein | | | |
| Q91X44 | GCKR_MOUSE | Gluco- | Gckr | Mus | 587 |
| | | kinase | | musculus | |
| | | regulatory | | (Mouse) | |
| | | protein | | | |
| Q91754 | GCKR_XENLA | Gluco- | gckr | Xenopus | 619 |
| | | kinase | | laevis | |
| | | regulatory | | (African | |
| | | protein | | clawed | |
| | | | | frog) | |

The invention provides crystals of (a) a glucokinase regulatory protein (GKRP) and of (b) deletion mutants, i.e. of truncated forms of GKRP as summarized above.

The human aminoacid sequence of the wildtype enzyme (hGKRP), relevant for the invention discussed here is derived from its accompanying DNA sequence published e.g. in SWISS-Prot. entry Q14397 (SWISS-Prot. being available via the URL http://www.uniprot.org; August 2011). The coding sequence for hGKRP is also given in SEQ ID NO. 1 of this application, the derived aminoacid sequence in SEQ ID NO. 2.

The mouse aminoacid sequence of the wildtype enzyme (mGKRP) has been identified from genome data. The coding sequence for mGKRP is given in SEQ ID NO. 3, the derived aminoacid sequence in SEQ ID NO. 4.

The respective sequences from rat (rGKRP) are derived from SWISS-Prot. entry Q07071. The coding sequence for rGKRP is given in SEQ ID NO. 5, the derived aminoacid sequence in SEQ ID NO. 6.

Nucleic acid sequences and aminoacid sequences can be compared with respect to their degree of homology, e.g. by way of an alignment of the sequences to be compared. According to the invention, the degree of homology is defined by a percentage of identity measured e.g. by a method as described in D. J. Lipman and W. R. Pearson in Science 227 (1985), p. 1435-1441. It is preferred to perform such a comparison by use of commercially available computer programs like Vector NTI® Suite 7.0, sold by Invitrogen/InforMax, Inc., Bethesda, USA, preferably by the preselected default parameters. The calculated homology value can refer to the sequences as a whole or for partial sequences only. A broader understanding of the term homology includes the similarity which includes conservative exchanges, i.e. of aminoacids with comparable chemical activities which most often determine the overall activity of the protein in a similar way. With respect to nucleotide sequences only the percentage of identity is used.

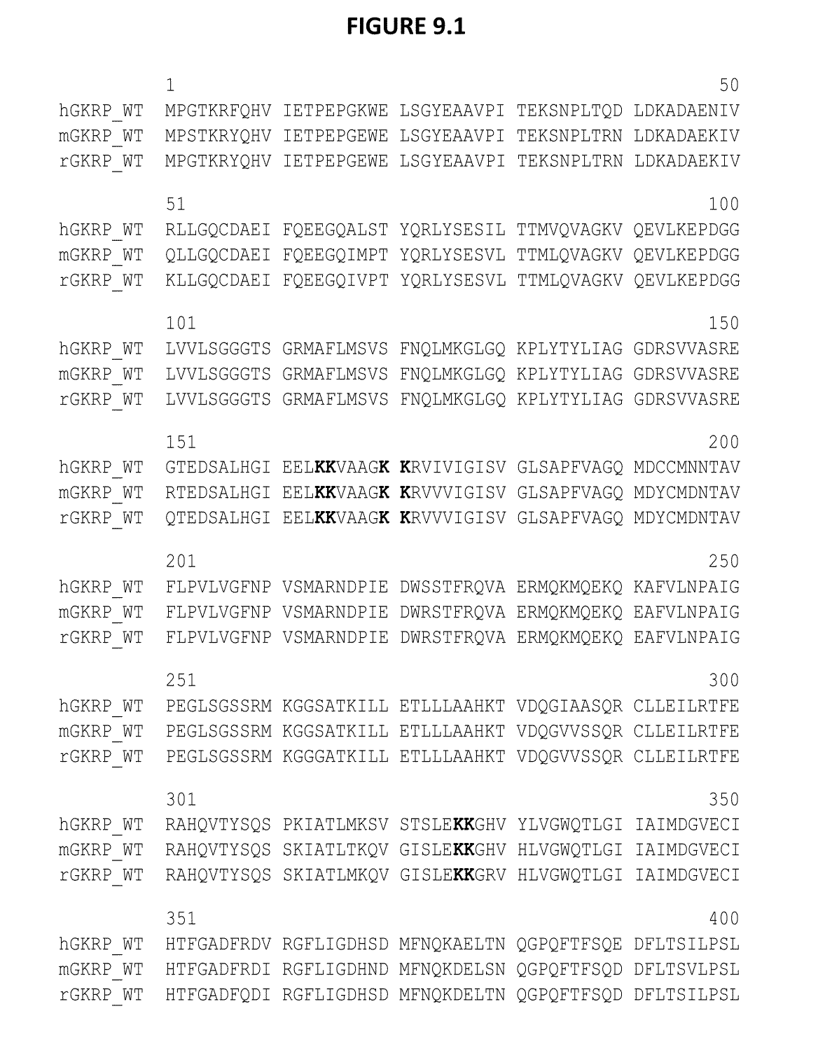

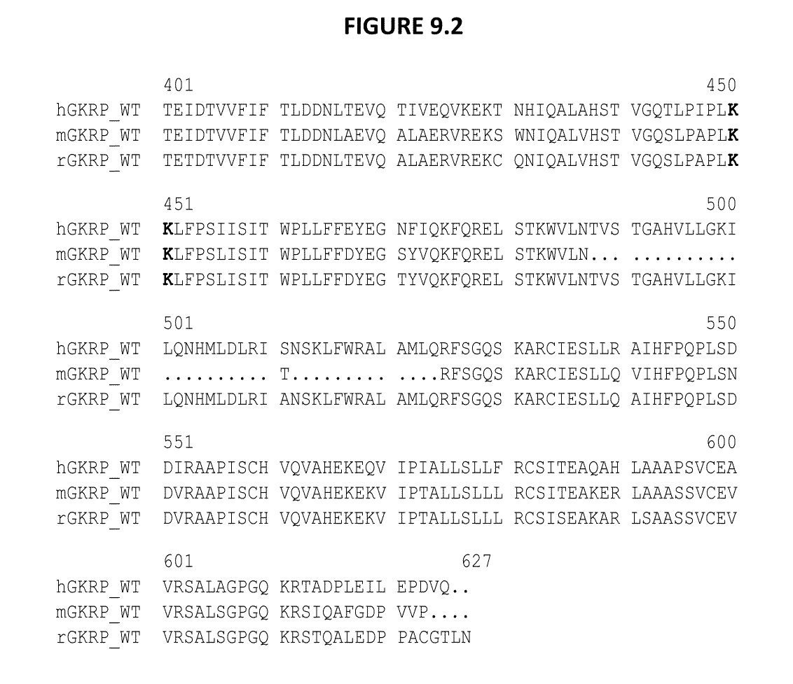

FIG. 9 shows an alignment of the aminoacid sequences of hGKRP (first line), mGKRP (second line) and of rGKRP (third line). By use of the mentioned computer program Vector NTI® Suite 7.0 with the preselected default parameters, the following homology ranges have been calculated:
hGKRP vs. mGKRP: 81.9% identity
hGKRP vs. rGKRP: 88.2% identity
mGKRP vs. rGKRP: 89.2% identity

The homology with the Xenopus aminoacid sequence has been calculated to be 58.2% identity (with human), 54.1% identity (with mouse) and 55.6% identity (with rat). On the other hand, no GKRP aminoacid sequences from other organisms have been found that are more closely related with hGKRP, mGKRP and/or rGKRP.

Accordingly, it lies well within the ambit of the actual invention to include all crystals of such GKRP proteins that are at least 82% identical with one or more of hGKRP, mGKRP and/or rGKRP, as disclosed under SEQ ID NO. 2, 4 and/or 6 of this application.

A second aspect of this invention pertains to crystals of (b) deletion mutants (truncated forms) of GKRP comprising (i) at least 82% identity to positions 6 to 606 of SEQ ID NO. 2, (ii) at least 82% identity to positions 6 to 606 of SEQ ID NO. 4 and/or (iii) at least 82% identity to positions 6 to 606 of SEQ ID NO. 6.

This is supported by two facts: On the one hand, especially the N- and/or C-terminus of a protein is very often solvent exposed and flexible over the more ordered structure of the remaining protein and thus hard to fix in a protein crystal. On the other hand, especially the termini are in many cases not essential for the biochemical function of the protein. Accordingly it is legitimate to reduce the protein to its core structure in order to allow crystallization, while the derived protein crystal and three-dimensional structure still give insight about the real structure of the protein and can thus be used for the intended purposes. To which extent, however, such termini can be cut off from the protein in order to ease crystallization depend on the peculiarities of the protein and needs to be analyzed in each specific case.

In the context of the underlying invention, the constructs listed in following Table 2 have experimentally proven to crystallize at an acceptable or good quality.

TABLE 2
Expression constructs of GKRP experimentally
proven to crystallize

| Construct | Type | Abbreviation | Quality |
|---|---|---|---|
| hGKRP(1-625)_C-His | Full length, | hGKRP(1- | acceptable |
| | human, | 625)_C-His | |
| | reference | hGKRP_{WT-His} | |
| mGKRP_C-His | orthologue | | acceptable |
| hGKRP(1-625)_C-strep2 | affinity tag | | acceptable |
| hGKRP(1-625)_G5_C- | affinity tag | | acceptable |
| His | | | |
| hGKRP(1- | Surface | hGKRP_{K326} | good; suitable |
| 625)_K326T_K327T | mutation | | for structure |
| | | | determination |
| hGKRP(1- | Surface | hGKRP_{K450} | acceptable |
| 625)_K450T_K451T | mutation | | |
| hGKRP(1-625)_K567T | Surface | | acceptable |
| | mutation | | |

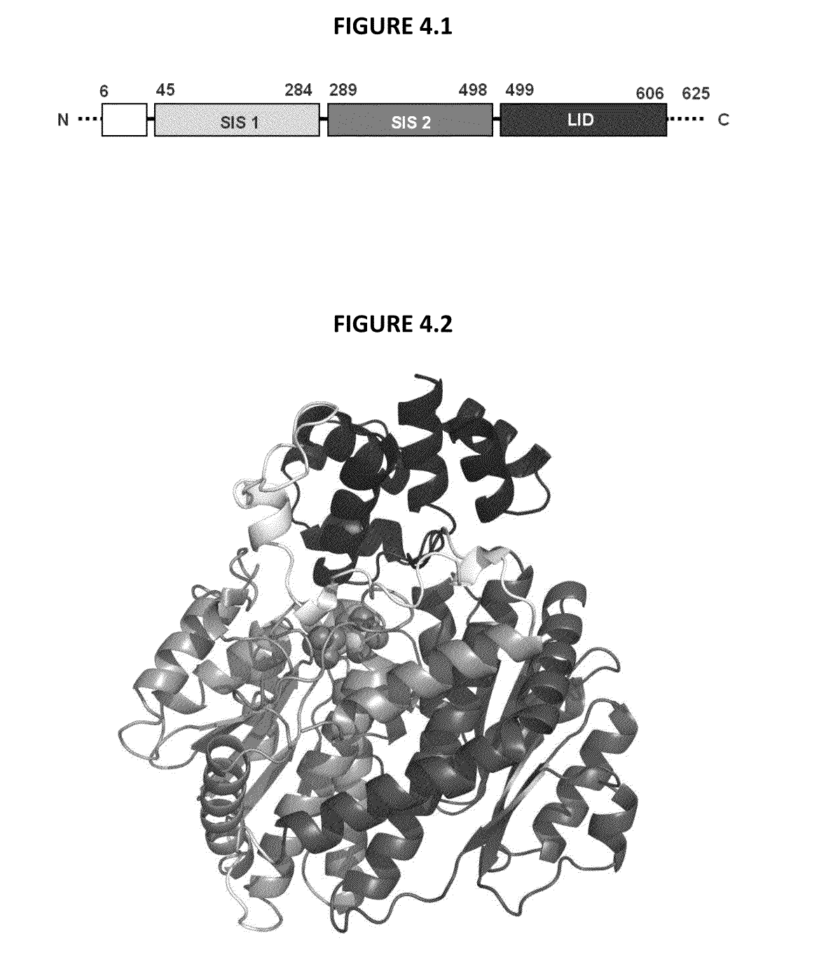

Two different crystals of human GKRP have been created as described in the examples of this application. Their common structure is shown in FIG. 4 which can be described as follows.

GKRP is trilobal in shape. It consists of two topologically identical sugar isomerase (SIS) domains of equal size, herein referred to as SIS-1 (residues 45-284) and SIS-2 (residues 289-498), respectively, capped by an alpha helical C-terminal domain (residues 499-606, termed LID-domain) which in turn is embraced by residues 6-44 of the N-terminus.

Below, secondary structure elements of SIS-1, SIS-2 and LID domain are designated with indices A, B and C, respectively. Each subdomain has an αβ structure and is dominated by a five-stranded parallel β sheet flanked on either side by α helices forming a three-layered αβα sandwich. Helices in the loops connecting β strands run approximately antiparallel to the strands. The SIS domain fold represents the nucleotide-binding motif of a flavodoxin type. In addition to this motif, there is a α helical extension of about 20 residues donated by the N-terminus of each subdomain (αA1, residues 46-61 of SIS-1 and αB1, residues 289-310 of SIS-2, respectively) which folds over the domain interface and onto the respective other domain. The two SIS domains are related by an approximate twofold axis going through the SIS domain interface which is build from helices αA1, αA3, and αA7 (SIS-1) and the corresponding helices of SIS-2 (αB1, αB3, and αB7). The two SIS domains can be superimposed with an rmsd of 1.7 Å for 129 equivalent a carbon atoms. The structural and topological similarity of the subdomains suggests that GKRP has evolved through a gene duplication step, similar to other SIS domain containing proteins.

The LID-domain is build from a bundle of 7 α-helices (αC1-7). Its core is build by a triple helical bundle (αC1, αC2, αC5) with an ubiquitin-like fold. The core is flanked by the C-terminal αC7 which stacks approximately parallel to the central bundle and by helices αC3, αC4 and αC6 which run approximately perpendicular. The LID-domain is initiated by a rather irregular peptide stretch (residues 499-512) where a short β-hairpin (residues 401-504) is the only secondary structure feature. These N-terminal 14 residues are wedged between the α-helical bundle that constitutes the core of the cap domain and the SIS-domain dimer, and contributes significantly to the cap-SIS interface.

Accordingly, GKRP crystals according to the invention are trilobal in shape, comprising two more or less equally sized SIS domains and one LID domain which in turn is embraced by a part of the N-terminus.

A dimerization via the SIS domains is possible. More preferred are monomers.

GKRP crystals according to the invention comprise those which are free from binding low molecular weight molecules as well as those which are complexed with certain low molecular weight molecules, especially natural interacting partners. Such crystals are described below in more detail.

In preferred modes, the invention pertains to a crystal according to aspect (a) (GKRP), wherein the GKRP comprises
(i) increasingly preferred at least 85, 90, 95, 97.5, 98, 99 and mostly preferred 100% identity to SEQ ID NO:2,
(ii) increasingly preferred at least 85, 90, 95, 97.5, 98, 99 and mostly preferred 100% identity to SEQ ID NO. 4 and/or
(iii) increasingly preferred at least 85, 90, 95, 97.5, 98, 99 and mostly preferred 100% identity to SEQ ID NO. 6, or
   to a crystal according to aspect (b) wherein the deletion mutant (truncated form) of GKRP comprises
(i) increasingly preferred at least 85, 90, 95, 97.5, 98, 99 and mostly preferred 100% identity to positions 6 to 606 of SEQ ID NO:2,
(ii) increasingly preferred at least 85, 90, 95, 97.5, 98, 99 and mostly preferred 100% identity to positions 6 to 606 of SEQ ID NO. 4 and/or
(iii) increasingly preferred at least 85, 90, 95, 97.5, 98, 99 and mostly preferred 100% identity to positions 6 to 606 of SEQ ID NO. 6.

The increasingly preferred identity values are calculated as explained above by way of an alignment of the sequences to be compared. According to the invention, the degree of homology is defined by a percentage of identity measured e.g. by the method as described in D. J. Lipman and W. R. Pearson in Science 227 (1985), p. 1435-1441. Such a comparison can be performed by use of commercially available computer programs like Vector NTI® Suite 7.0, sold by Invitrogen/InforMax, Inc., Bethesda, USA, preferably by the preselected default parameters. The calculated homology value can refer to the sequences as a whole for aspect (a) and for the complete partial sequences as defined by aspect (b).

As can be seen from the examples, crystals derived from the complete hGKRP sequence with just little sequence variations could successfully be made in accordance with the invention. Because of the high sequence homology between the examined species human, mouse and rat including large identical stretches (compare FIG. 9) it can be expected that related GKRP proteins form crystals under the same or similar conditions. Further it can be expected that with increasing identity with the wildtype sequences, i.e. with SEQ ID NO. 2, 4 and/or 6 the information about the native structure and the exerted biochemical activities will be more predictive. This especially applies to the intended use of the crystal and/or its structural data for the identification of small molecular compounds that could interact with respective parts of the protein in vivo.

The same applies mutatis mutandis to the deletion mutants (truncated forms) of GKRPs of aspect (b) because the respective deletion mutants are expected to give more robust crystals and thus more confident structural data than the complete sequences with still predictive value for the binding and enzymatic characteristics of GKRP in its in vivo environment.

In one preferred form the invention pertains to such crystals, wherein the GKRP or the deletion mutant (truncated form) of GKRP comprises point mutations selected from 1 to 20 additional aminoacids, added to the C- and/or N-terminus (tags), preferably 1 to 10 additional aminoacids, added to the C- and/or N-terminus (tags).

Especially preferred are those, wherein the GKRP or the deletion mutant (truncated form) of GKRP comprises one or more of the tags selected from: 1 to 10 additional Histidins added to the N-terminus (His-tag), optionally with a linker of 1 to 5 additional aminoacids, and/or 1 to 10 additional Histidins added to the C-terminus (His-tag), optionally with a linker of 1 to 5 additional aminoacids.

Such point mutations are e.g. helpful for stabilizing the protein structure in solution and thus ameliorate the crystallization process. N- or C-terminal extensions, especially the mentioned tags ease the purification of the respective proteins, e.g. by affinity chromatography and are thus helpful for the preparation of sufficient amounts for the crystallization process. On the other hand, such point mutations and/or extensions are expected to have basically no negative influence on the structure of the protein crystal itself so that they will still be predictive for the in vivo situation of the analyzed GKRP.

Especially preferred are also those, wherein the GKRP or the deletion mutant (truncated form) of GKRP comprises 6 additional Histidins added to the C-terminus, with a linker of one aliphatic and one acidic aminoacid, preferred a C-terminus defined by the octapeptide LEHHHHHH or VEHHHHHH.

As supported e.g. by the accompanying examples, such proteins can be purified by affinity chromatography via a immobilized metal (e.g. nickel-)chelates.

One preferred mode of the invention is a crystal according to the aspects before, wherein the GKRP or the deletion mutant (truncated form) of GKRP comprises deletions of 1 to 50 aminoacids from the N-terminus (N-terminal truncation) and/or from the C-terminus (C-terminal truncation) of the non-tagged GKRP or of the deletion mutant (truncated form) of GKRP, preferably a deletion of the N-terminal 44 aminoacids in the numbering according to SEQ ID NO. 2 and/or of the C-terminal 20 aminoacids in the numbering according to SEQ ID NO. 2.

For it has been found advantageous to delete these stretches from the respective termini in order to allow a well ordered crystal structure which is still predictive for the protein's in vivo function.

Another mode of the invention resides in such crystals wherein the GKRP or the deletion mutant (truncated form) of GKRP comprises point mutations selected from 1 to 15 deletions or substitutions of solvent exposed aminoacids.

This is based on the fact that especially solvent exposed aminoacids have an influence on the physicochemical behaviour of the protein, esp. during the crystallization process. For example polar or ionic groups might interfere with the same ionic groups on the surface of neighbour proteins thus hindering an easy crystallization. Accordingly, it has been found advantageous to delete these aminoacids or to exchange them e.g. to non-polar or non-ionic groups.

Based on this teaching, preferred modes of the invention reside in such crystals, comprising one or more of the following substitutions of solvent exposed aminoacids: K164T, K165T, K170T, K171T, K326T, K327T, K450T, K451T, K567T, in the numbering according to SEQ ID NO: 2 and FIG. 9, preferably K326T and/or K327T, more preferred K326T and K327T.

This is exemplified by the present disclosure. The analyzed K326T/K327T double mutation is located on the surface of the SIS-2 domain at the end of helix αB2. The region is neither involved in contacts to SIS-N, or the active site, nor does it interact with the LID-domain or the N-terminus. Biochemically, GKRP_{K326} behaves identical to wild type GKRP and it can thus be assumed that all conclusions drawn from the mutant structure are valid for wild type GKRP as well. Despite the improvement that the K326T/K327T mutation made on crystal quality, there are only modest involvments in crystal contacts: Thr327 is solvent exposed and not involved in contacts to neighboring molecules at all. The Thr326 sidechain is found in two conformations, one of which makes two interactions with a symmetry related molecule (denoted by a *): a van der Waals interactions of Thr326 CG2 with Asn197* and a hydrogen bond of OG1 to water W283, which in turn contacts Thr198*.

Much preferred, within this aspect of the invention, are such crystals wherein the GKRP or the deletion mutant (truncated form) of GKRP is selected from: hGKRP (SEQ ID NO. 2), mGKRP (SEQ ID NO. 4), rGKRP (SEQ ID NO. 6), hGKRP_C-His (SEQ ID NO. 8), hGKRP_C-His_K326T/K327T (SEQ ID NO. 10), mGKRP_C-His (SEQ ID NO. 12) and rGKRP_C-His (SEQ ID NO. 14), preferably hGKRP_C-His_K326T/K327T (SEQ ID NO. 10).

One preferred mode of the invention pertains to crystals, wherein the GKRP or the deletion mutant (truncated form) of GKRP is complexed with a low molecular weight binding ligand in the active site, preferably with a low molecular weight binding ligand selected from Fructose-1-Phosphate (F1P), Fructose-6-Phosphate (F6P), Orthophosphate (Pᵢ) and Sorbitol-6-Phosphate (S6P), preferably Fructose-1-Phosphate (F1P) or Orthophosphate (Pᵢ).

This has been found advantageous with respect to the natural function of the protein which is much influenced by the interaction with its natural binding partners, especially in the active site, or with close homologs to them. This allows an easier crystallization. It further allows more reliable data about the in-vivo situation. This is especially useful with respect to the identification of other small molecule weight compounds that might substitute these partners or might only be desired to bind to the conformations of GKRP which are only formed in contact with the mentioned low molecular weight binding ligands.

The success of this approach is demonstrated by the examples of this application.

One highly preferred mode of the invention is such a crystal, wherein the GKRP or the deletion mutant (truncated form) of GKRP is hGKRP_C-His_K326T/K327T (SEQ ID NO. 10), and the low molecular weight binding ligand in the active site is selected from Fructose-1-Phosphate (F1P) and Orthophosphate (Pᵢ).

The success of the combined approach of C-terminal extension, exchange of solvent-exposed aminoacids and complexing with a low molecular weight binding ligand in the active site is demonstrated by the examples of this application.

One not less preferred mode of the invention is such a crystal, wherein the GKRP or the deletion mutant (truncated form) of GKRP is not complexed with a low molecular weight binding ligand in the active site, except one or more molecules of water and/or one or more of one atom cations, preferably one or more of water, magnesium ions (Mg²⁺) and/or calcium ions (Ca²⁺).

Such crystals are expected to give an alternative realistic insight into the in-vivo situation of the protein, e.g. in a non-active conformation. This might be useful to understand the changes in the protein's three-dimensional structure during its activity and might support the design of other small molecular weight molecules that interact especially with this form of the protein.

One preferred mode of the invention is such a crystal, wherein the active site of GKRP or the deletion mutant (truncated form) of GKRP is formed by one or more of the aminoacid residues or H₂O molecules selected from Arg518, Leu515, His351, Lys514, Asn512, Ser183, Glu153, Glu348, Gly181, Ala184, Ser179, Arg259, Gly107, Val180, Thr109, Ser110, Ser258, Gly108, Ile178, a H₂O molecule complexed by Arg518 and His351, a H₂O molecule complexed by Gly153 and Ser183, a H₂O molecule complexed by Arg259 and Ser258, a H₂O molecule complexed by Thr109 and a H₂O molecule complexed by Gly107 and Ile178, preferably by one or more of the aminoacid residues selected from Lys514, Asn512, Glu153, Gly181, Ser179, Val180, Gly107, Ser110, Thr109, Glu348, wherein all numbers refer to SEQ ID NO. 2.

This is supported by the fact that especially these side chains and complexed molecules are responsible for the three-dimensional structure of the active site of GKRP and are thus highly predictive for its function and possible interacting partners. Whereas it might be useful to exchange some aminoacid side chains of the protein, as explained above, it is expected by the teaching of this aspect of the invention, that especially these side chains should not be changed in order to reach a predictive model for the activity of the protein.

This is further supported by the examples of this application: GKRP was crystallised in the presence of fructose-1P (Kd=1 μM (rat GKRP)), which acts in a competitive manner with fructose-6P (Kd=20 μM (rat GKRP)) on mammalian GKRPs likely through a single binding site (rat GKRP: Kd (F6P)=20 μM; Kd (F1P)=1 μM)) (Van Schaftingen E., 1989; Veiga-da-Cunha and Van Schaftingen E., 2002). Clear ligand electron density indicates that αβ-D-fructose-1P binds in the pyranose configuration at the edge of the β-sheet of the SIS-1 domain. The binding site is formed by 3 loops (residues 107-109, 179-184, and 256-258) and one face of helix αA3″ (Glu150 and Glu 153). One loop (residues 179-183) embraces the phosphate group, whereas the other three polypeptides bind the fructose moiety. Terminal phosphate oxygens each form three hydrogen bonds with Ser110 and Ser179 (hydroxyl groups), Val180 and Gly181 (mainchain amino groups) and with water molecules (with low B-factors) tightly bound in the pocket. The dipole of helix αA5 directed to the phosphate seems additionally favourable for binding of fructose-1P. The binding site is complemented by one helix of SIS-2 (αB4, residues Glu348 and His 351) and one edge of the LID-domain (residues 512-518). The Lys514 amino group compensates one negative charge of the phosphate by interacting with oxygen O14 (3.3 Å) and with phosphoester O12 (2.9 Å). Hydroxyl substituents of fructopyranose are involved in polar contacts to residues from SIS-1 (Thr109 backbone NH; Glu153, carboxylate OE1), SIS-2 (Glu348, carboxylate OE2), the LID-domain (Lys514-NZ, Asn512-ND2) as well as two water molecules. When bound to phosphate instead of F1P, GKRP_{K326} assumes a conformation almost identical to GKRP_{K326}-F1P (0.14 Å rmsd on all Cα atoms). The lacking sugar moiety is replaced by several water molecules, but otherwise there are no significant deviations in the active site architecture. Despite the internal twofold symmetry of the SIS domains, GKRP contains only one ligand binding site, namely that in SIS-1, with the bound F1P. Another putative binding site at the equivalent region in SIS-2 is not occupied.

One preferred mode of the invention is such a crystal, wherein the GKRP or the deletion mutant (truncated form) of GKRP comprises a fructose-phosphate binding site at the interface between a SIS domain and a 2^{nd} α-helical domain with ubiquitin-like fold.

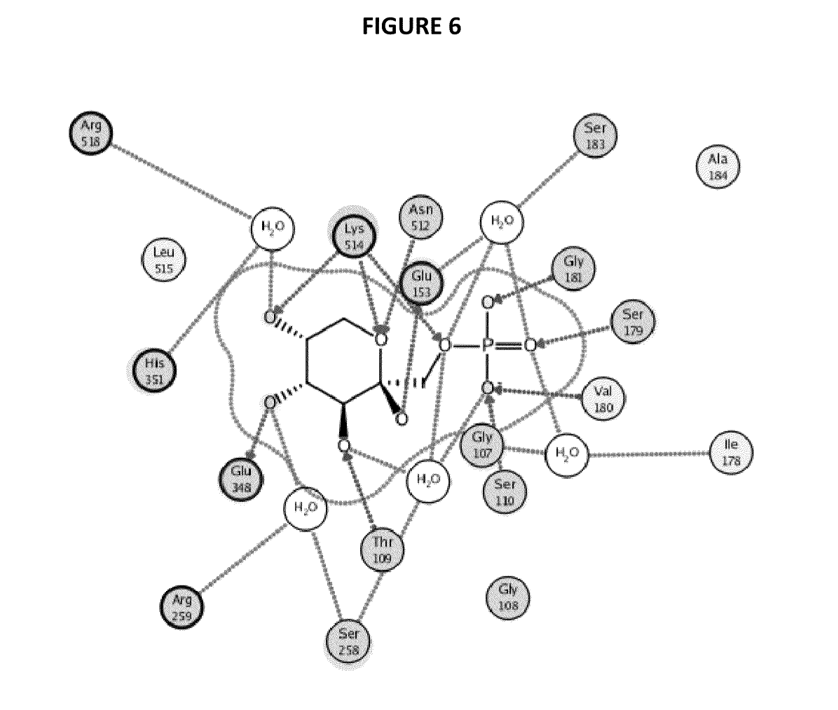

A specifically preferred mode of this aspect is illustrated by FIG. 6. The aminoacids depicted there are to be understood as the ones that define the relevant interface. Their identity was confirmed by example 7 (see below). Even more preferred are structures with the contacting partners of this region as listed in detail in table 3; even more preferred are the distances mentioned therein.

TABLE 3
The fructose-phosphate binding site at the interface
between a SIS domain and a 2^{nd} α-helical domain with
ubiquitin-like fold; further illustrated by FIG. 6.

| | | |
|---|---|---|
| Source atoms | Target atoms | Distance (Å) |

Sugar:

| | | |
|---|---|---|
| F1p 701A O2 | Lys 514A NZ | 3.13 |
| F1p 701A O10 | Glu 153A OE1 | 2.79 |
| F1p 701A O11 | Thr 109A N | 2.90 |
| | Wat 10W O | 2.68 |
| F1p 701A O7 | Lys 514A NZ | 2.80 |
| | Wat 3W O | 2.73 |
| F1p 701A O8 | Glu 348A OE1 | 2.88 |
| | Wat 104W O | 2.66 |
| | Glu 348A OE2 | 2.67 |

Phosphate-ester:

| | | |
|---|---|---|
| F1p 701A O12 | Lys 514A NZ | 2.90 |
| | Wat 20W O | 3.25 |

Phosphate:

| | | |
|---|---|---|
| F1p 701A O16 | Wat 20W O | 2.81 |
| | Wat 1W O | 2.85 |
| | Ser 179A OG | 2.60 |
| F1p 701A O14 | Gly 181A N | 2.77 |
| | Wat 135W O | 2.60 |
| F1p 701A O15 | Wat 10W O | 2.76 |
| | Ser 110A OG | 2.63 |
| | Val 180A N | 2.84 |

Sugar (van-der-Waals):

| | | |
|---|---|---|
| F1p 701A C9 | Lys 514A NZ | 3.94 |
| | Wat 10W O | 3.72 |
| | Glu 153A OE1 | 3.47 |
| | Glu 153A OE2 | 3.92 |
| | Wat 20W O | 3.57 |
| | Gly 107A O | 3.59 |
| F1p 701A C3 | Lys 514A NZ | 3.85 |
| | Glu 153A OE1 | 3.51 |
| F1p 701A C4 | Lys 514A NZ | 3.89 |
| | Ser 258A OG | 3.76 |
| | Wat 10W O | 3.30 |
| F1p 701A C5 | His 351A CE1 | 3.85 |
| | Wat 104W O | 3.92 |
| | Glu 348A OE2 | 3.52 |
| F1p 701A C6 | His 351A CE1 | 3.67 |
| | His 351A NE2 | 3.51 |
| | Lys 514A NZ | 3.84 |
| | Wat 3W O | 3.57 |
| F1p 701A C1 | Asn 512A CG | 3.95 |
| | Leu 515A CD1 | 3.97 |
| | Lys 514A CE | 3.79 |
| | Lys 514A NZ | 3.87 |
| | Asn 512A ND2 | 3.19 |

One preferred mode of the invention is such a crystal with the space group P2₁2₁2₁.

This is supported by the examples of this application. Further it can be expected that similar proteins, e.g. from other organisms within the homology range defined above will assume the same space group. Accordingly the crystals exemplified herewith will help to create further comparable crystals.

Highly preferred modes of the invention pertain to crystals according to the invention with unit cell dimensions between 60.0 and 62.0 Å for a, between 71.5 to 73.5 Å for b, and between 136.0 and 139.0 Å for c, preferably
(i) with the space group P2₁2₁2₁ and/or unit cell dimensions of a=61.0 Å, b=72.3 Å and c=136.9 Å.
(ii) with the space group P2₁2₁2₁ and/or unit cell dimensions of a=60.8 Å, b=72.2 Å and c=138.0 Å.

Crystals according to aspect (i) are exemplified by the hGKRP_C-His_K326T/K327T-Fructose-1-phosphate complex (F1P) of example 6 (table 2). Crystals according to aspect (ii) are exemplified by the hGKRP_C-His_K326T/K327T-phosphate complex (Phosphate) of example 6 (table 2). Accordingly it can be expected that further successfully producible crystals lie within these defined ranges, regardless of their exact aminoacid sequence and/or their organism of origin.

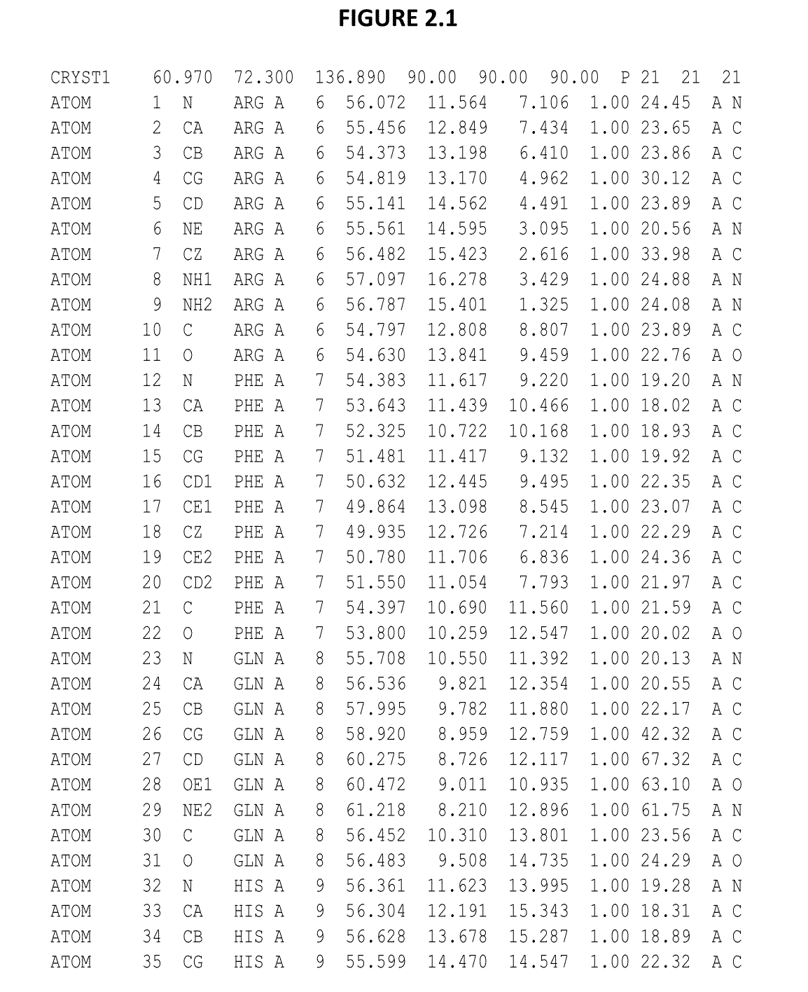

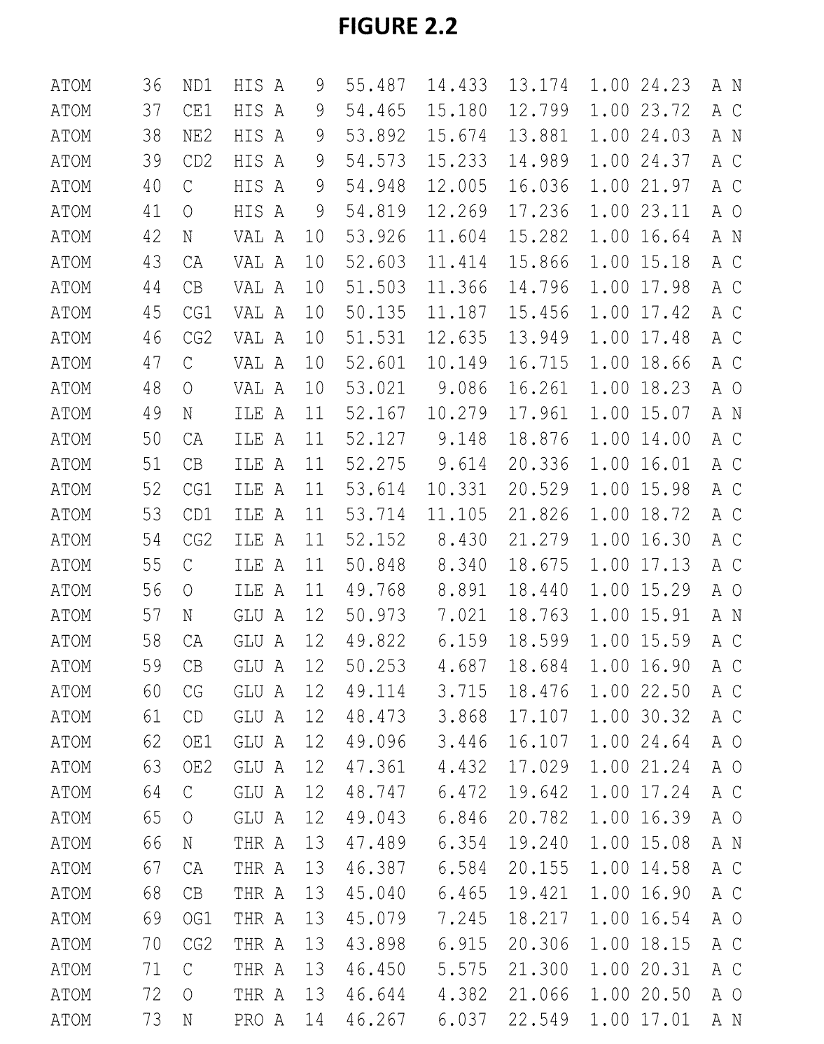

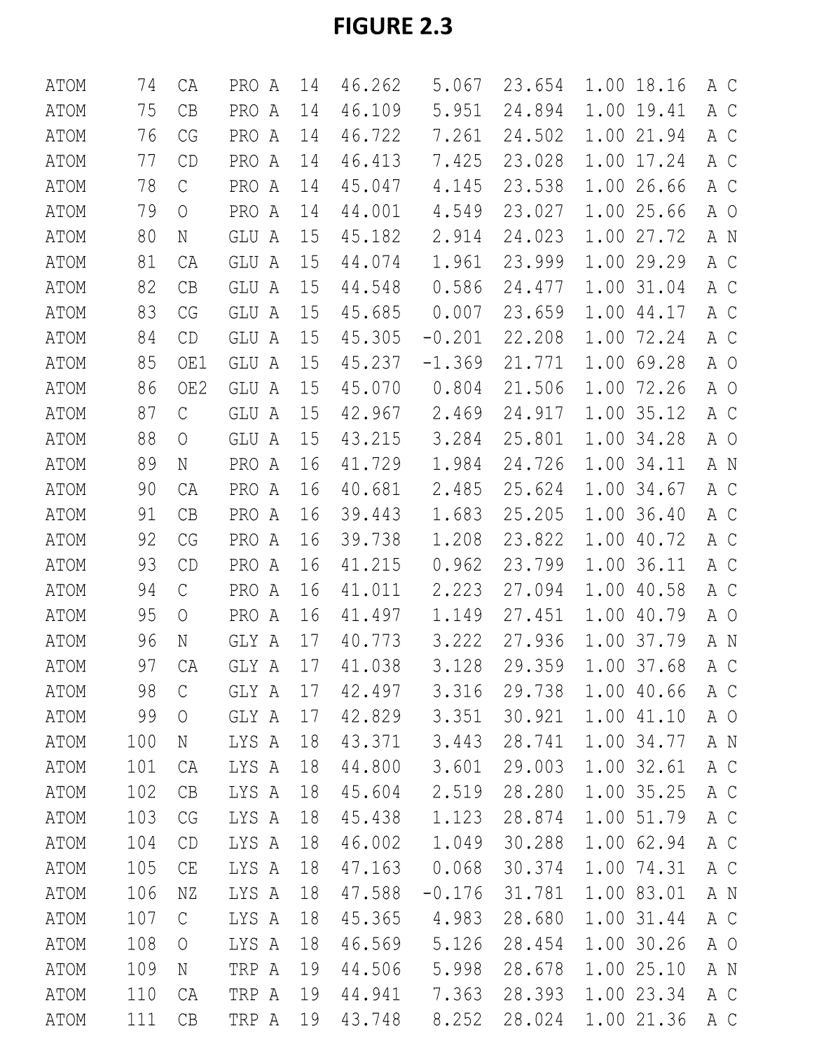

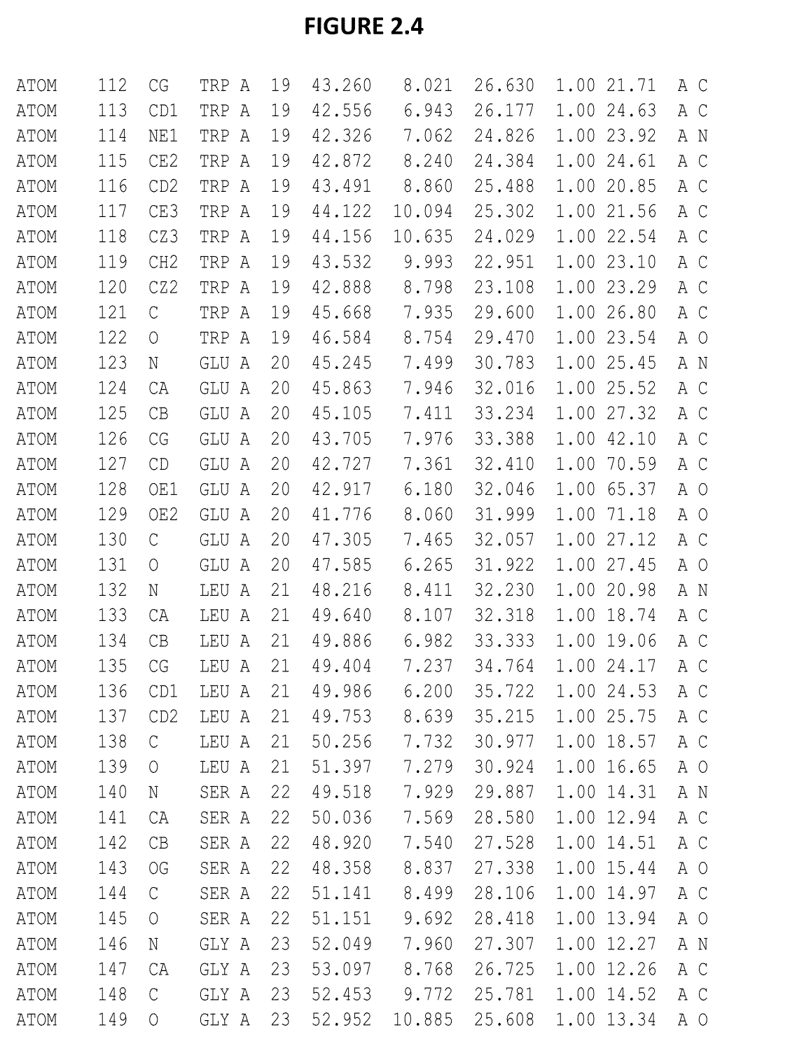

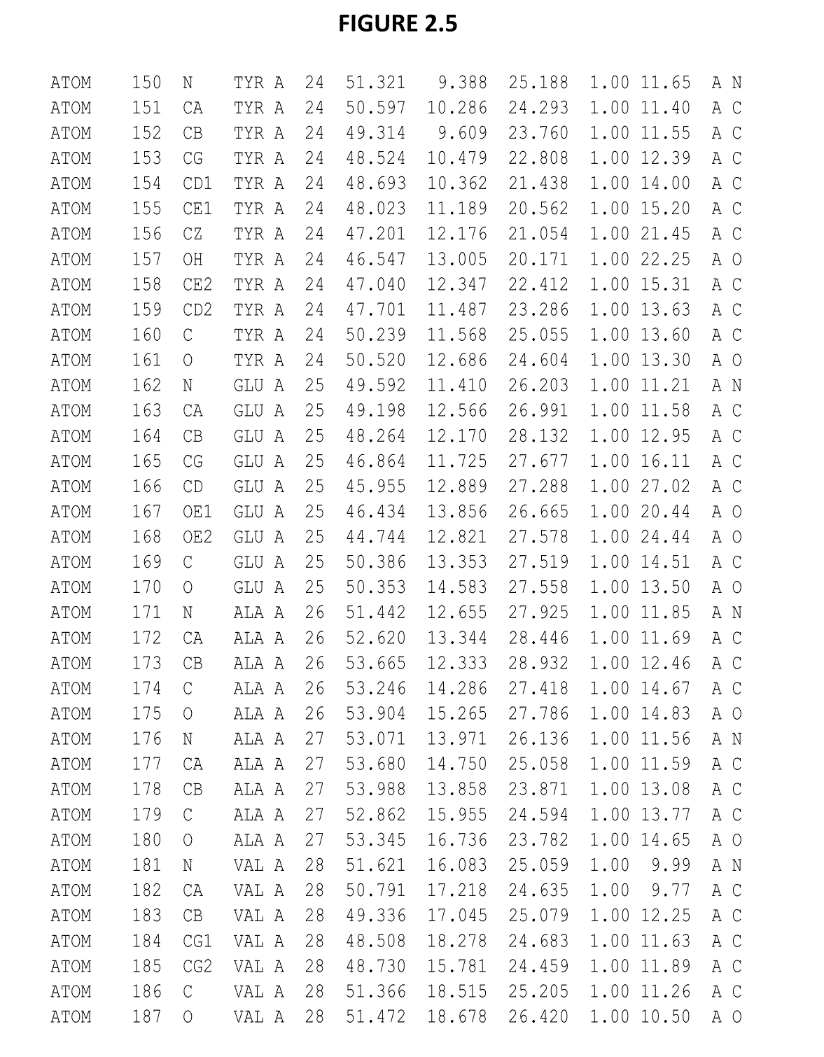

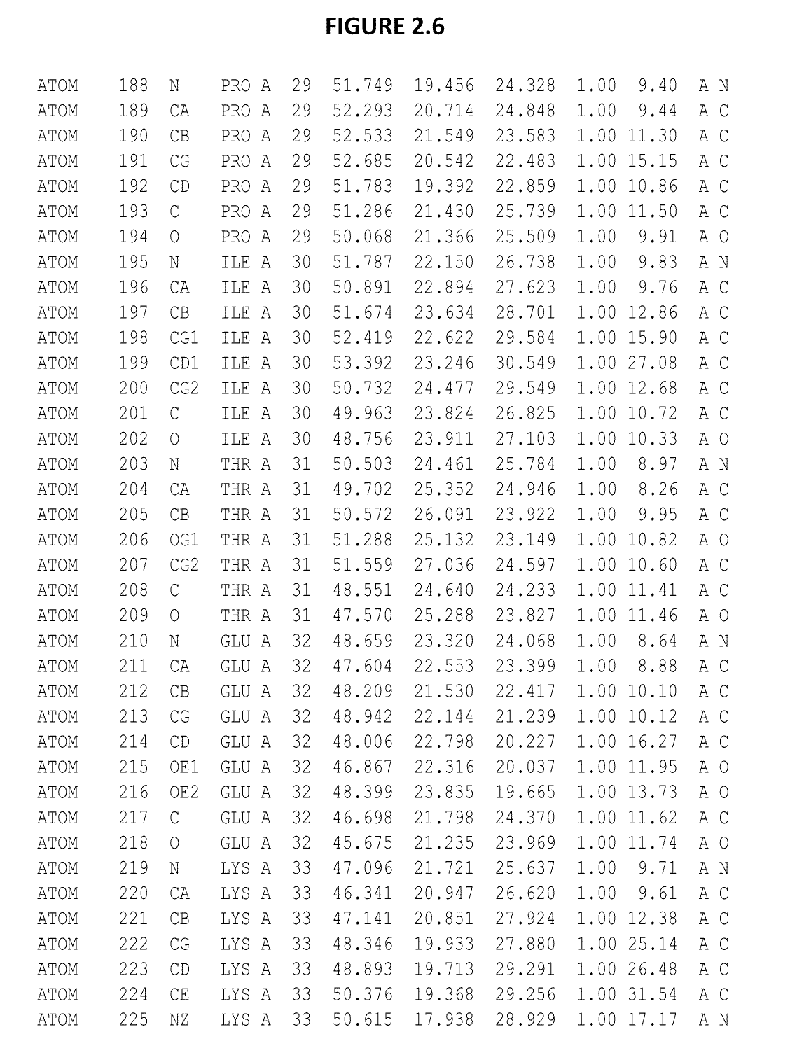

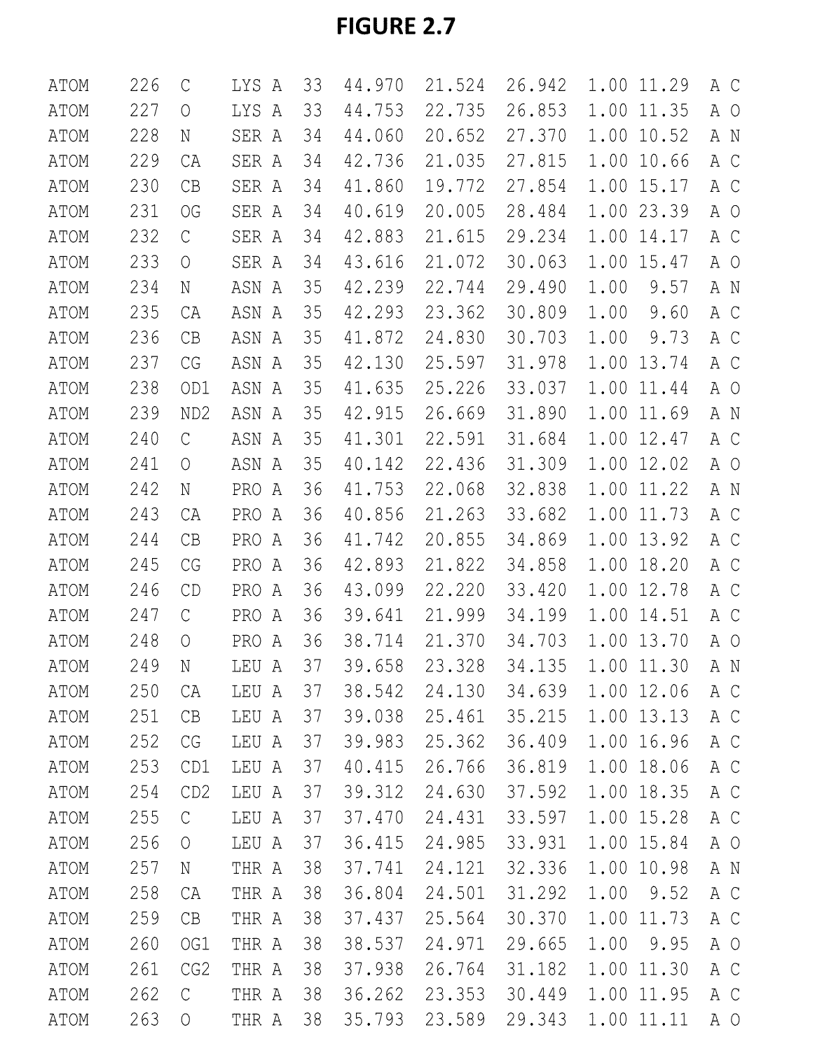

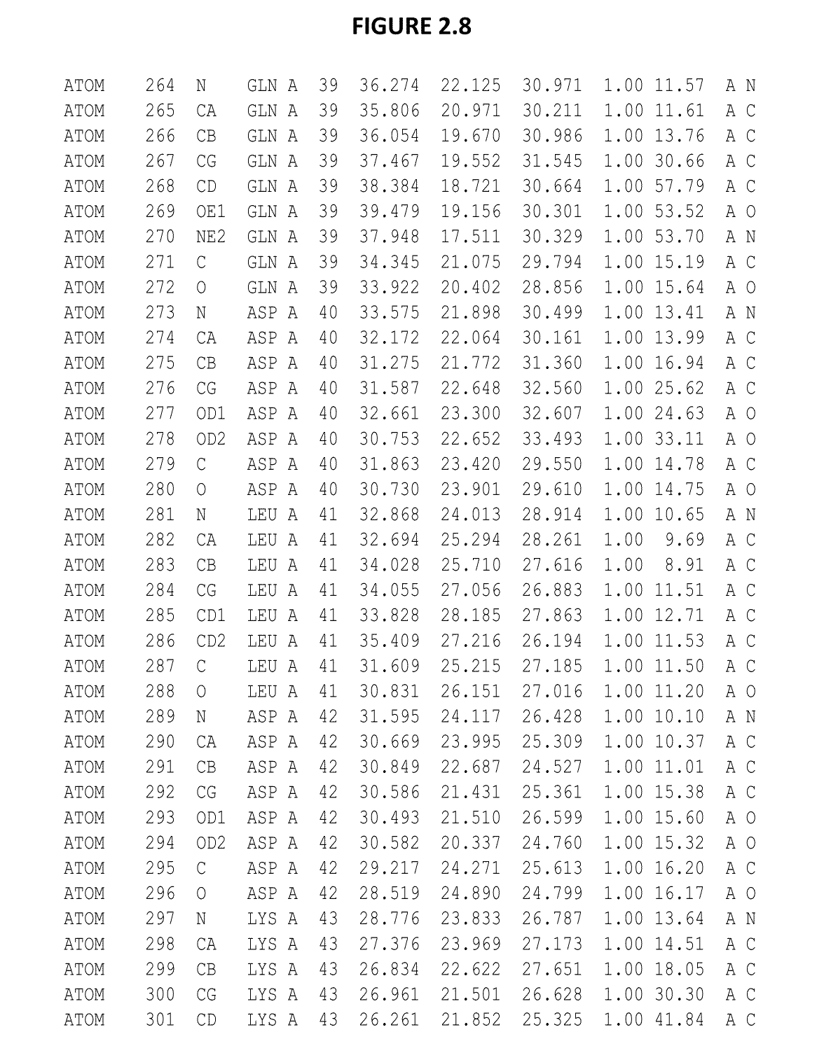

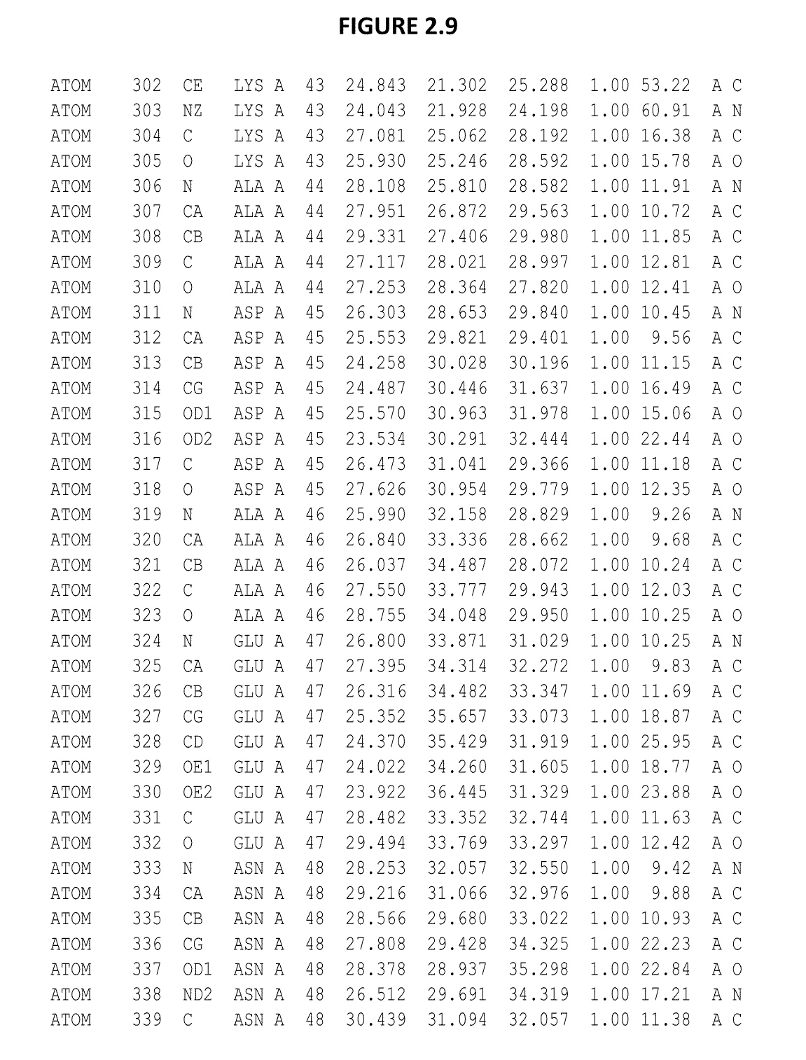

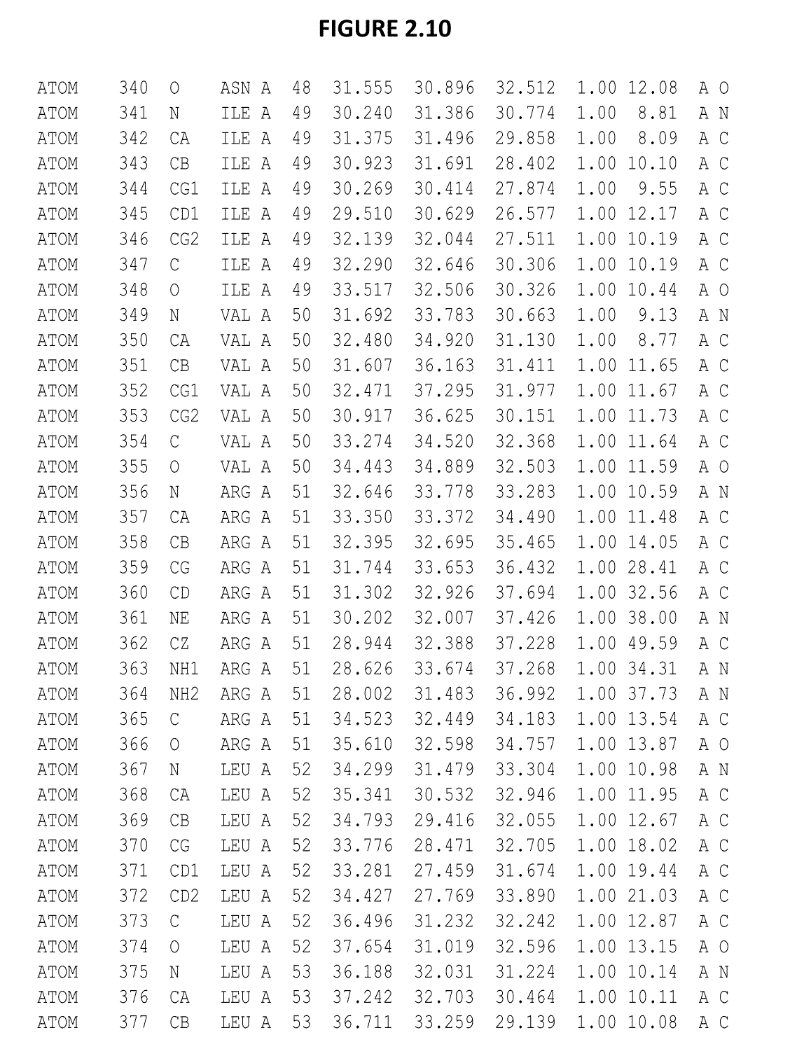

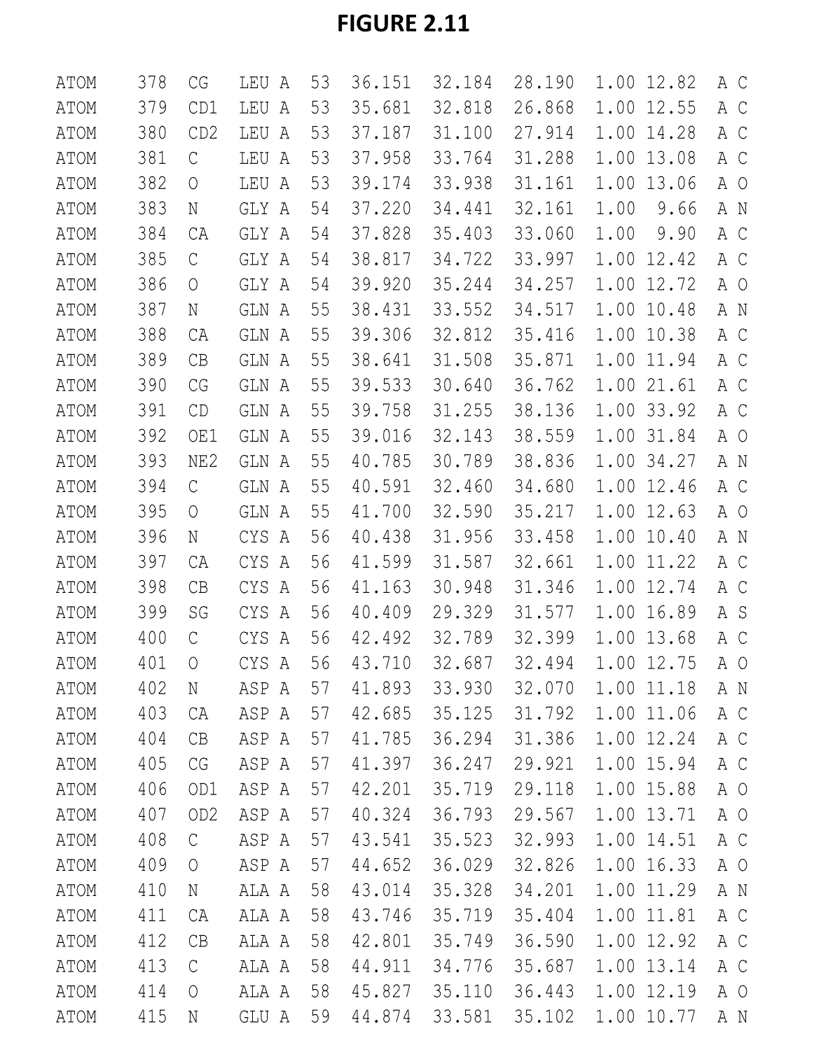

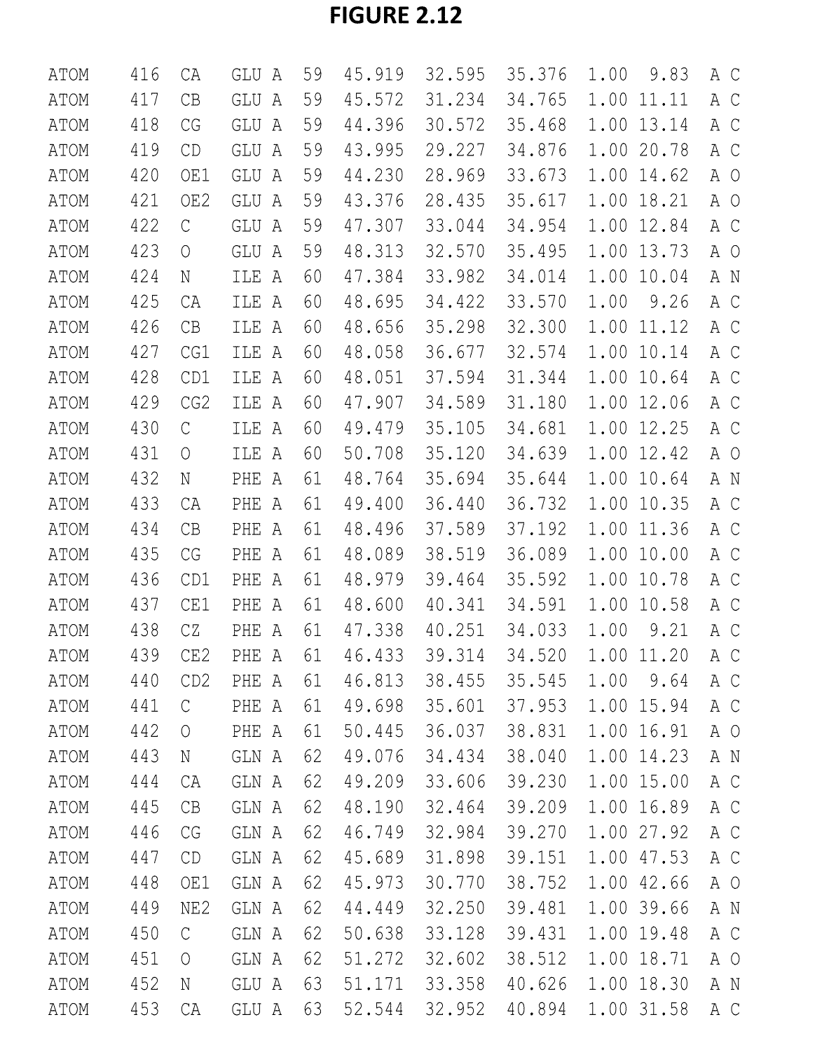

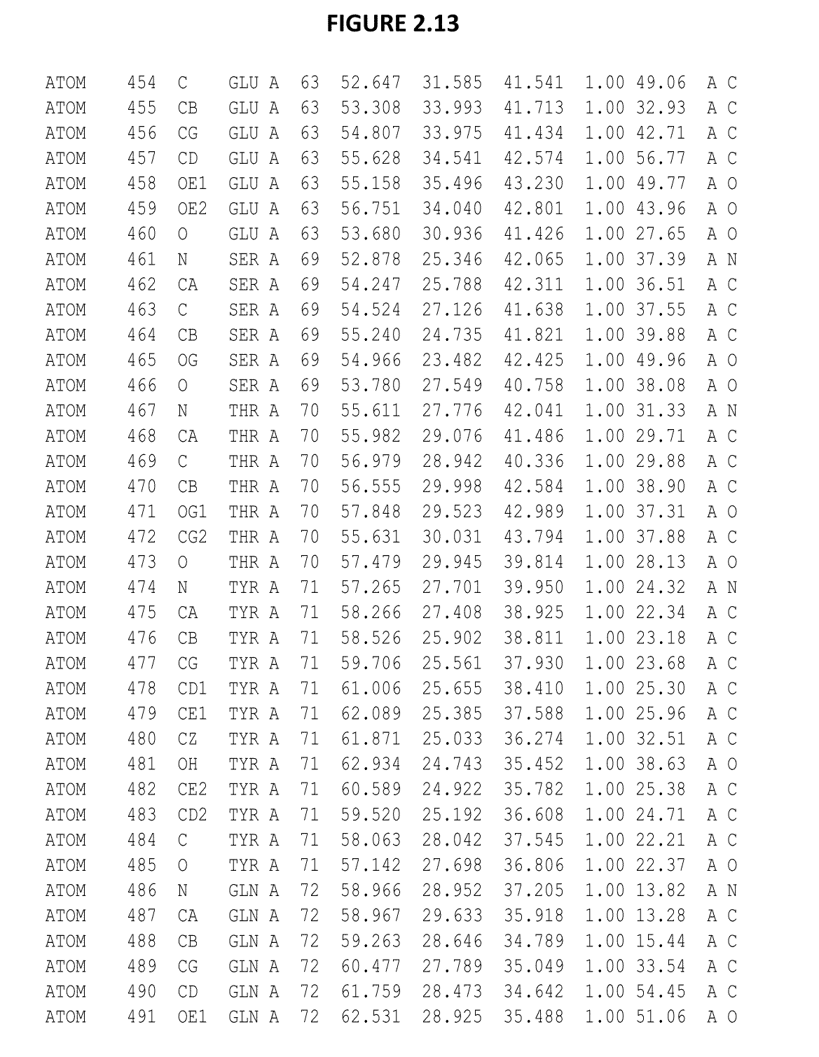

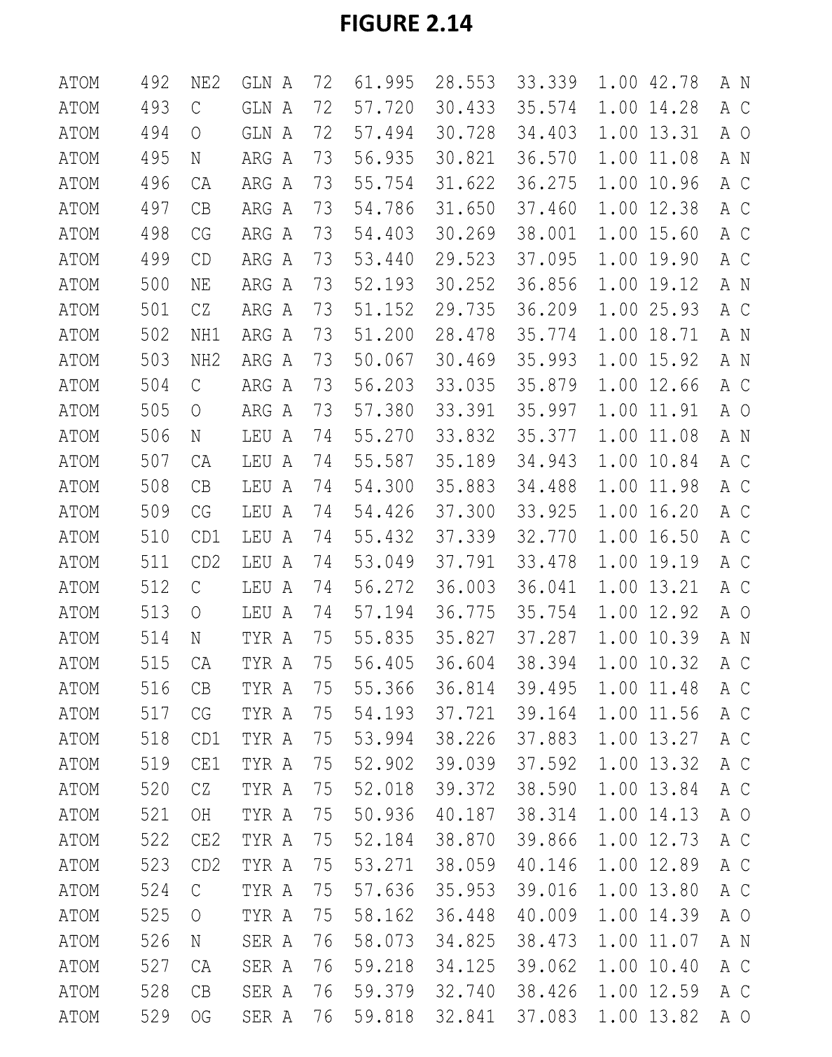

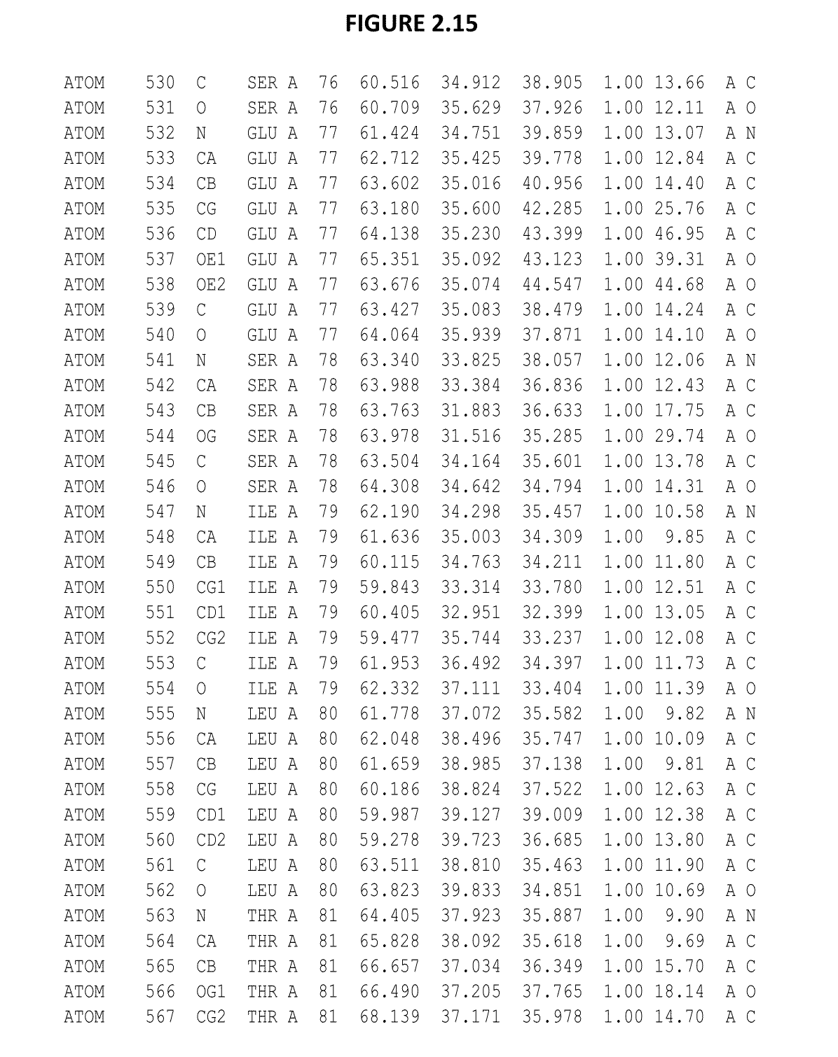

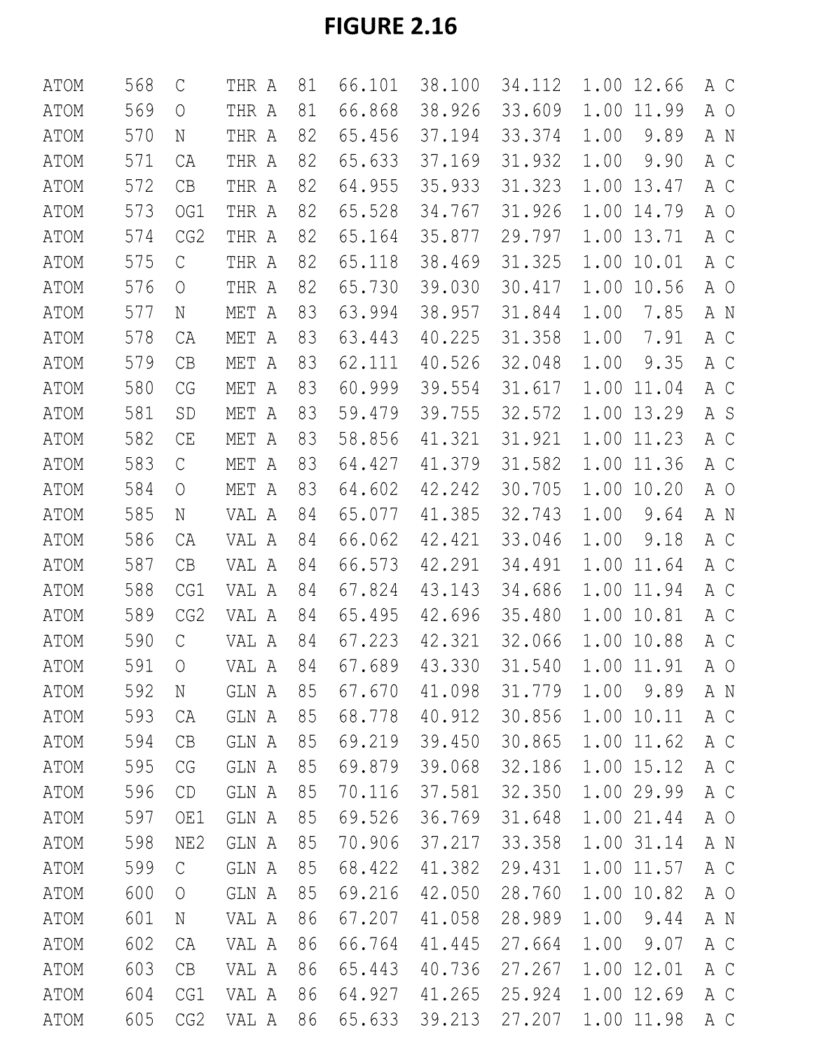

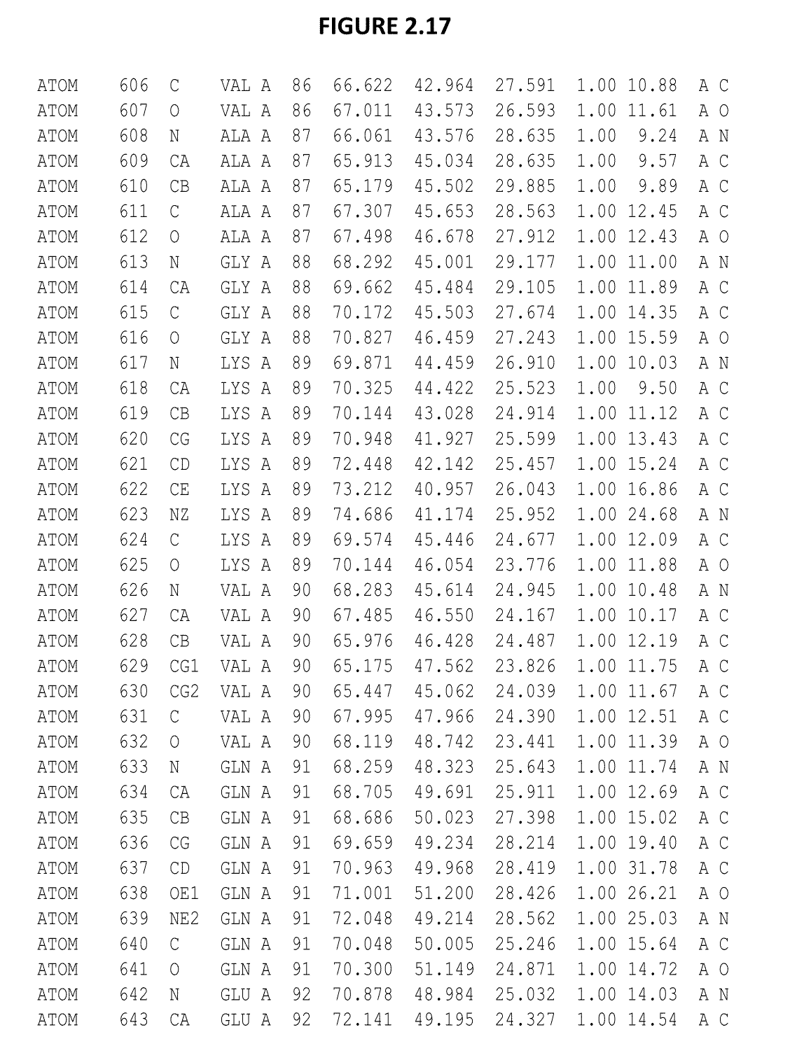

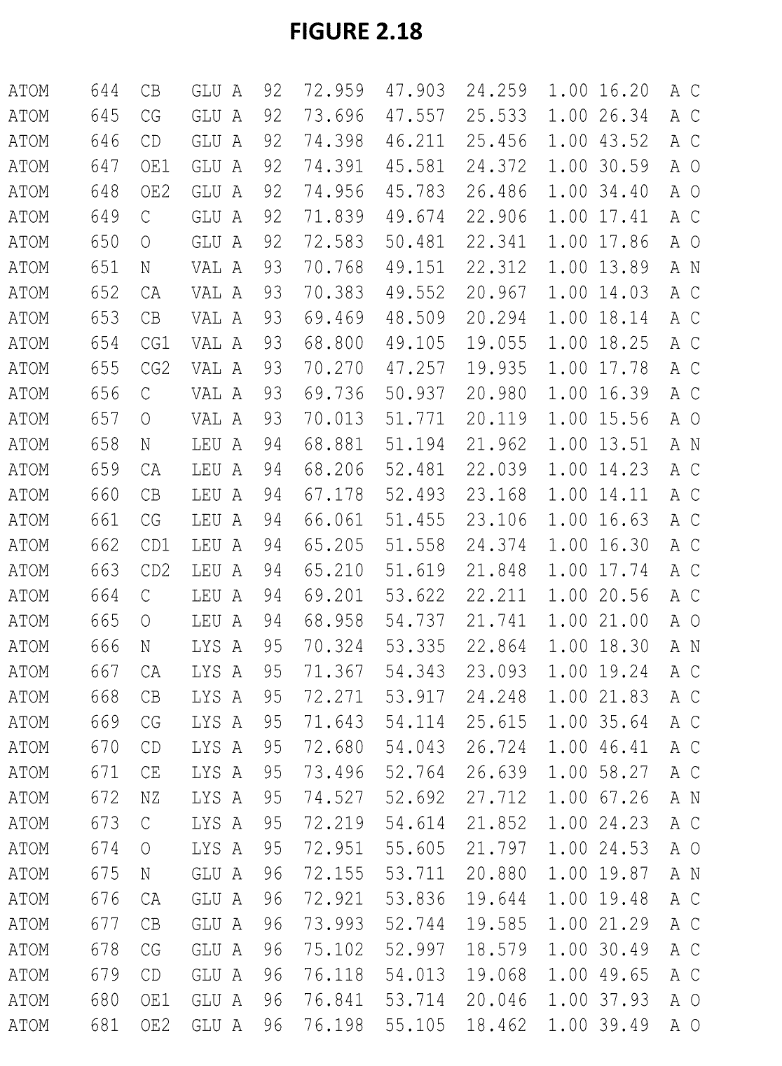

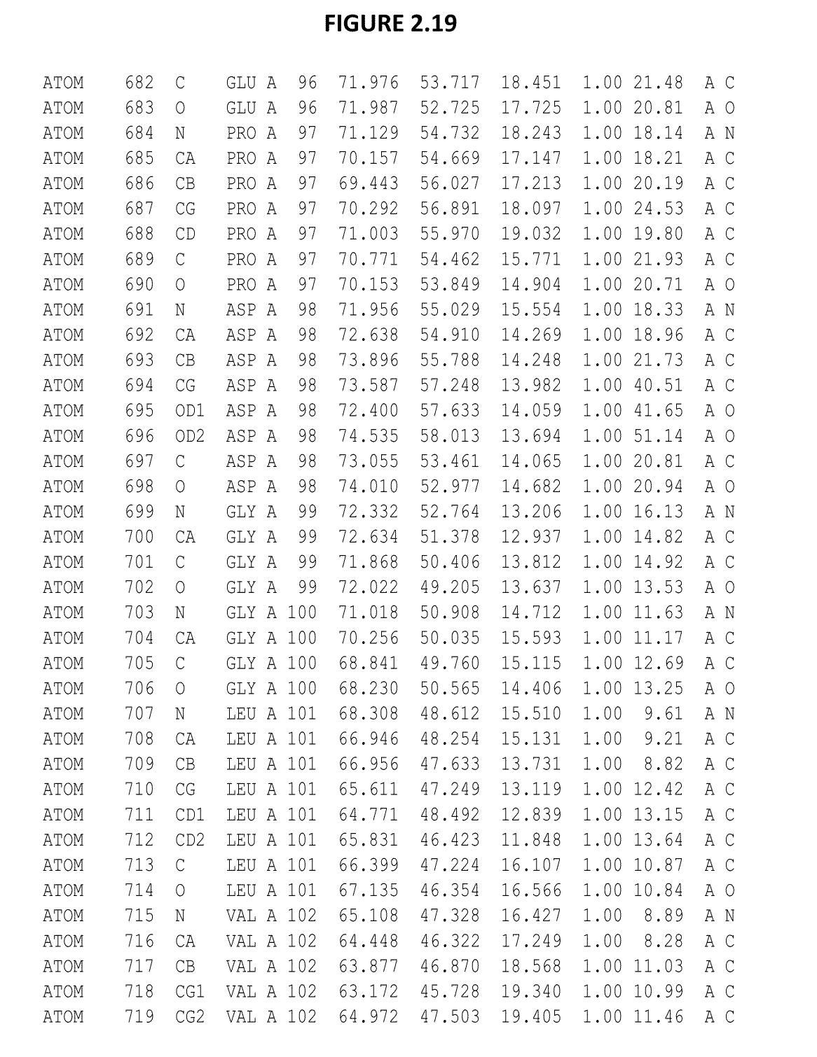

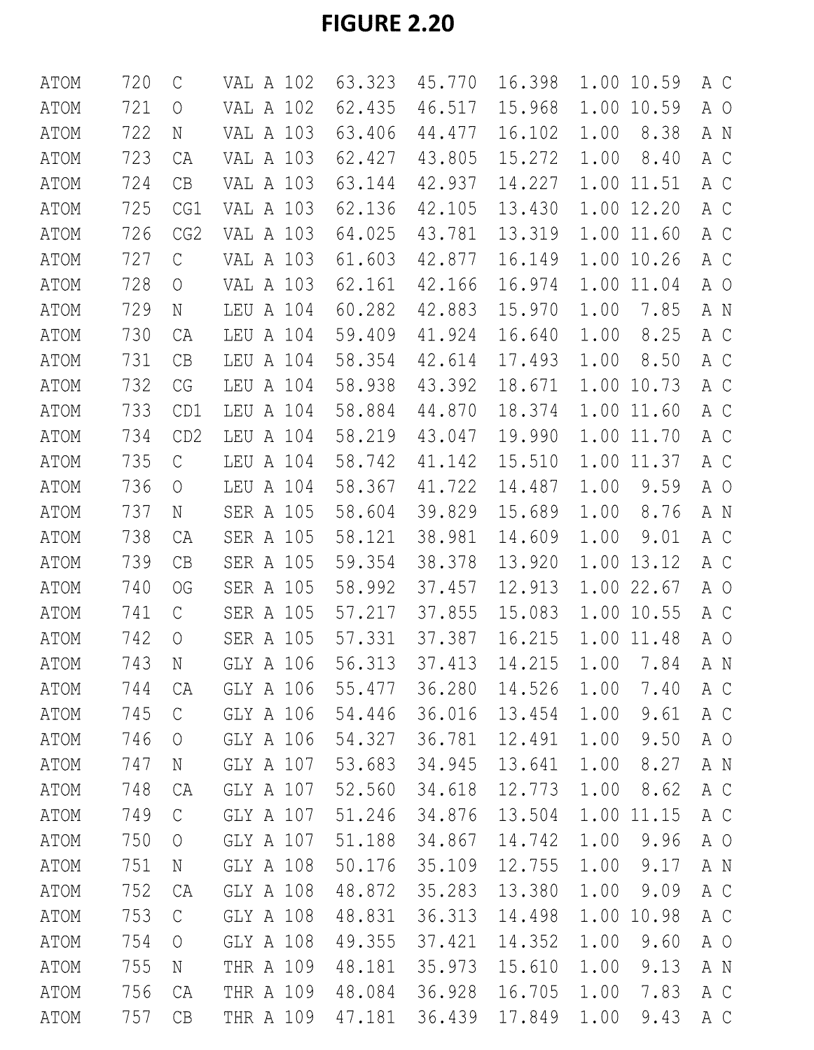

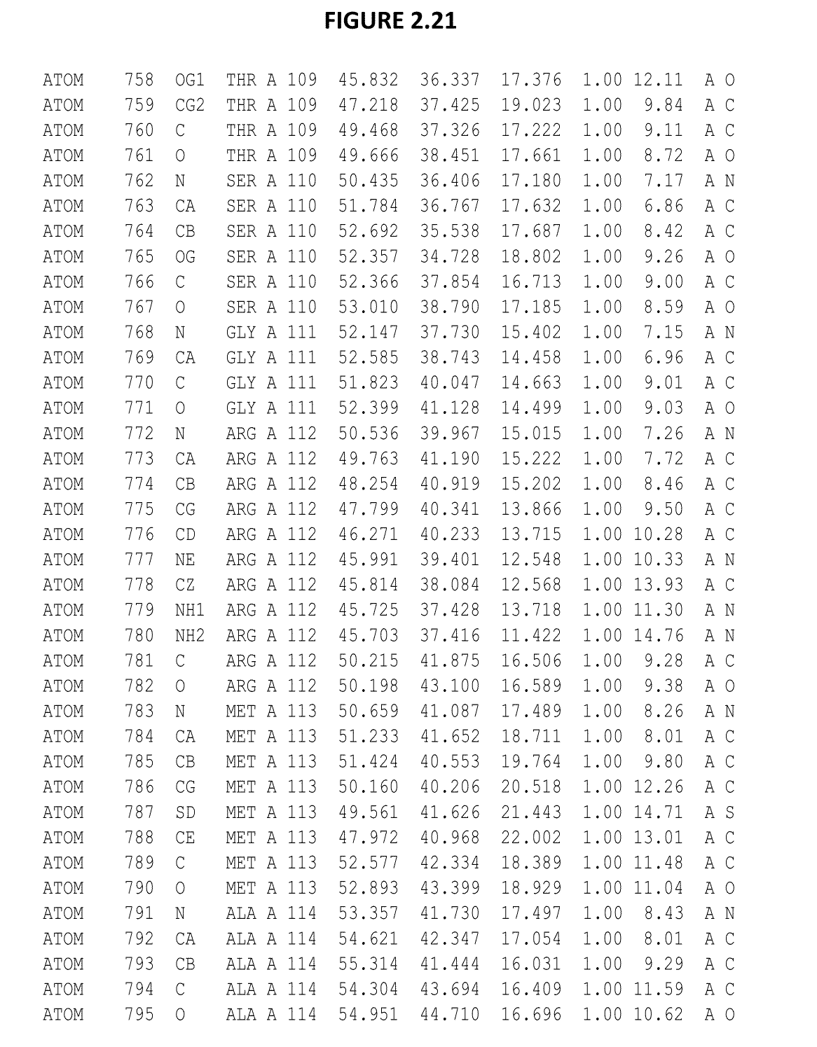

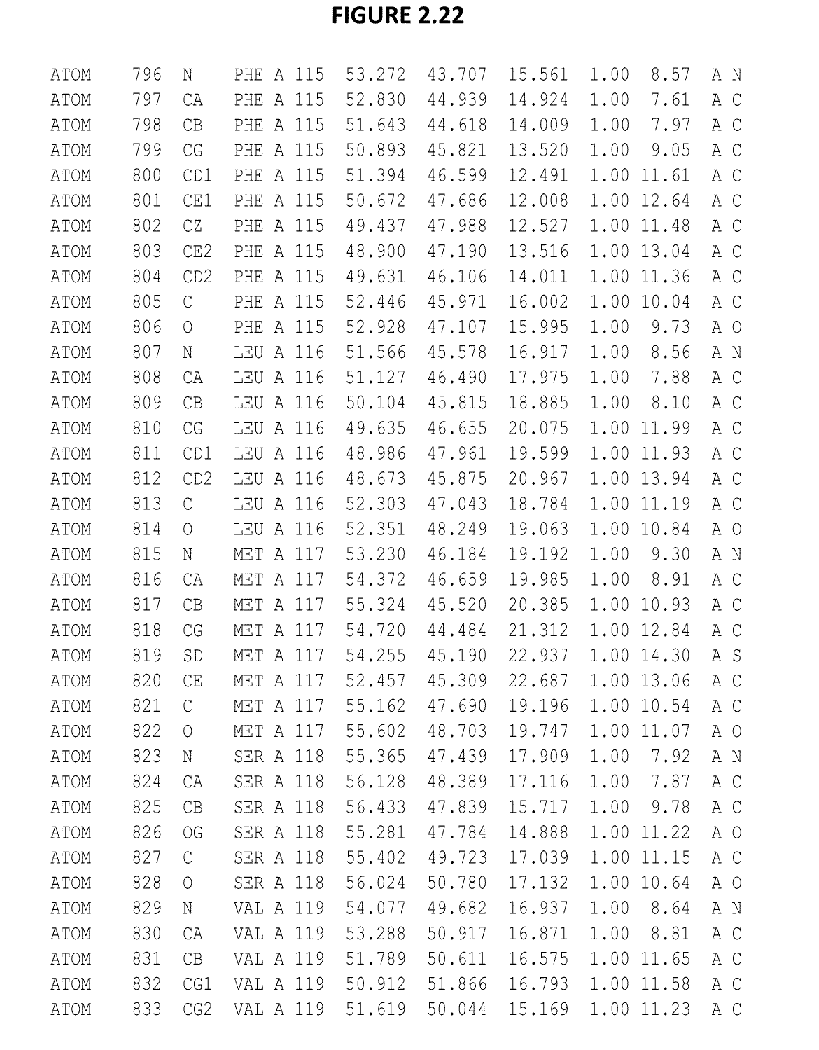

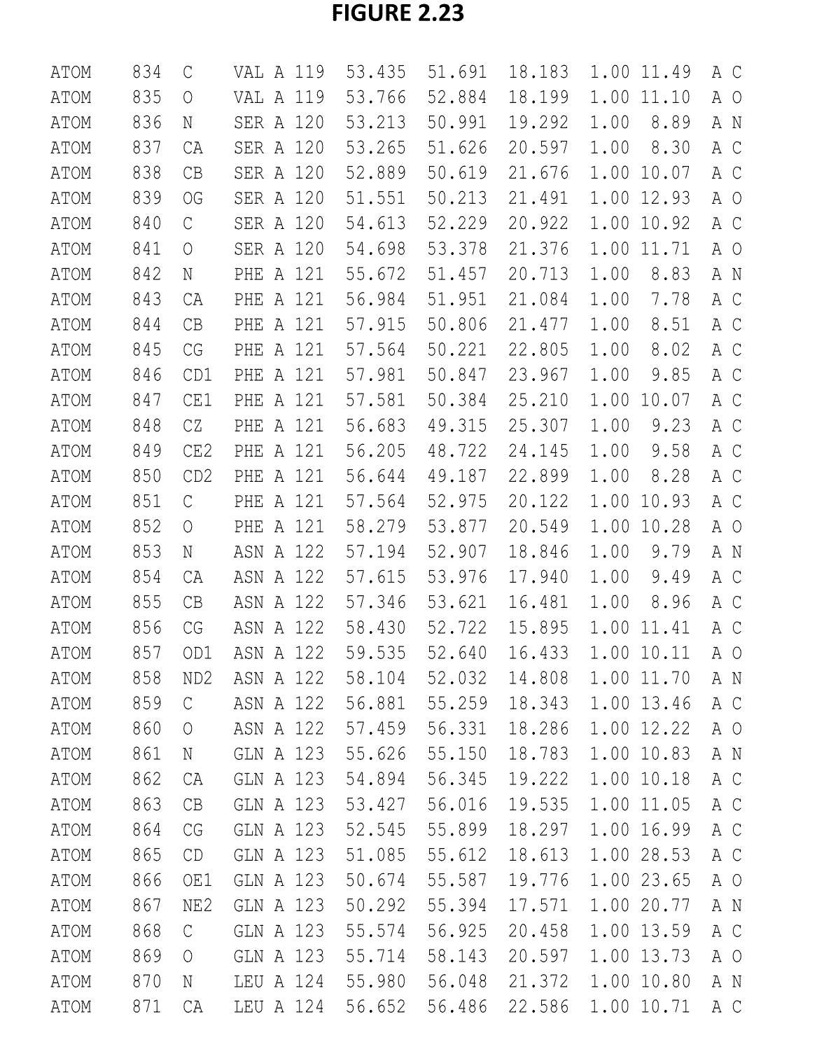

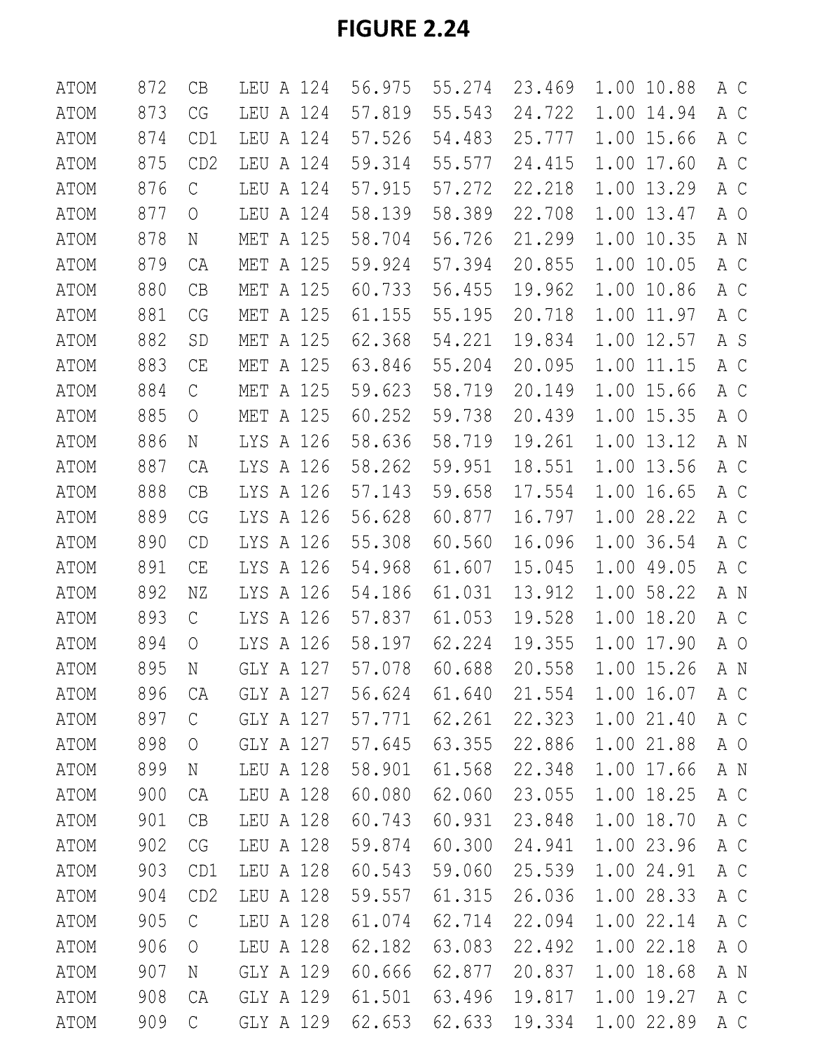

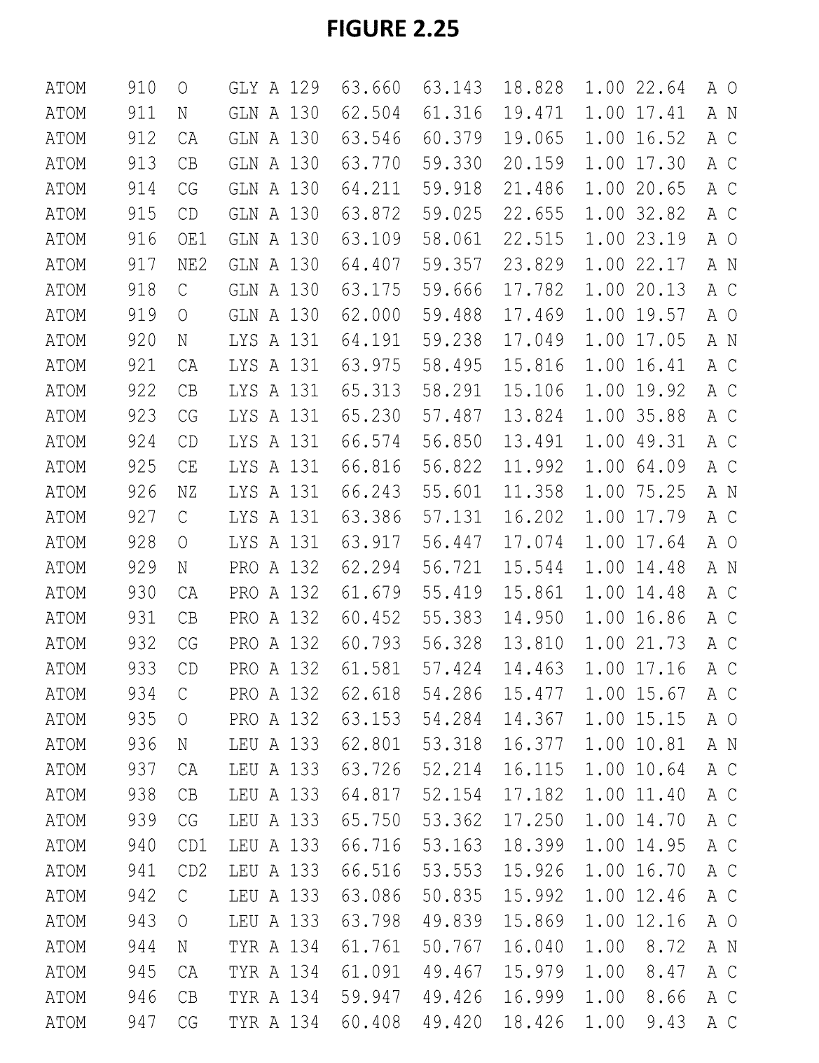

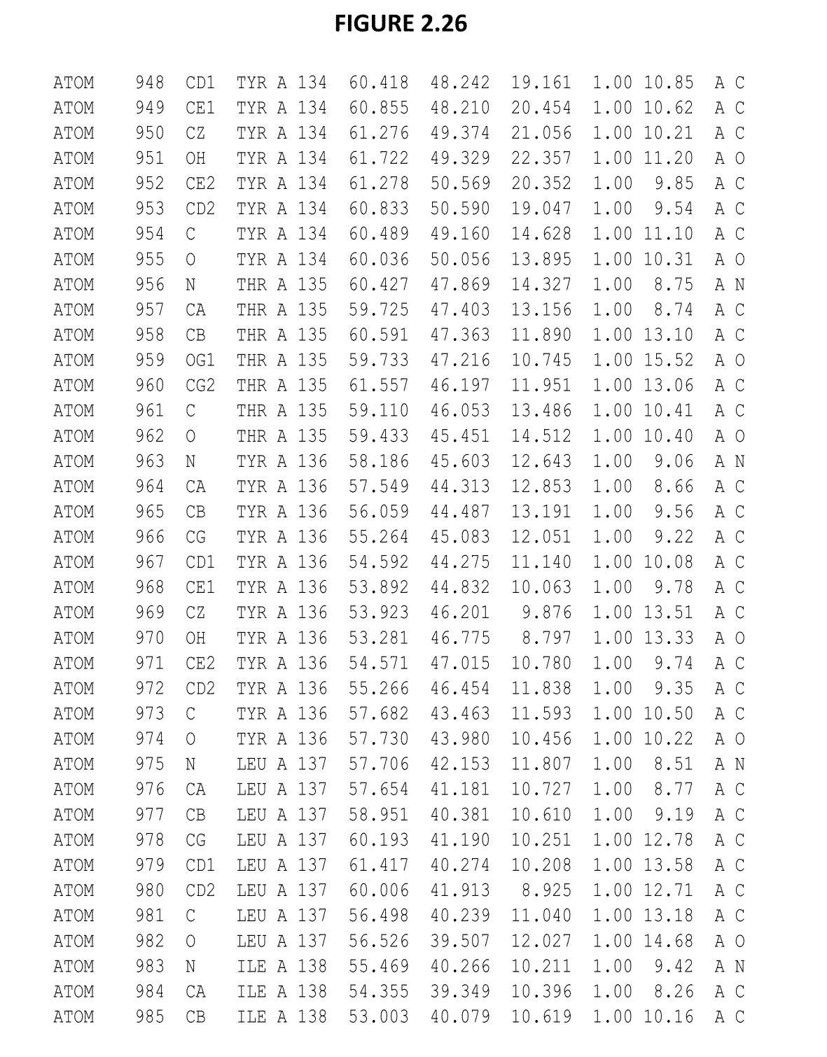

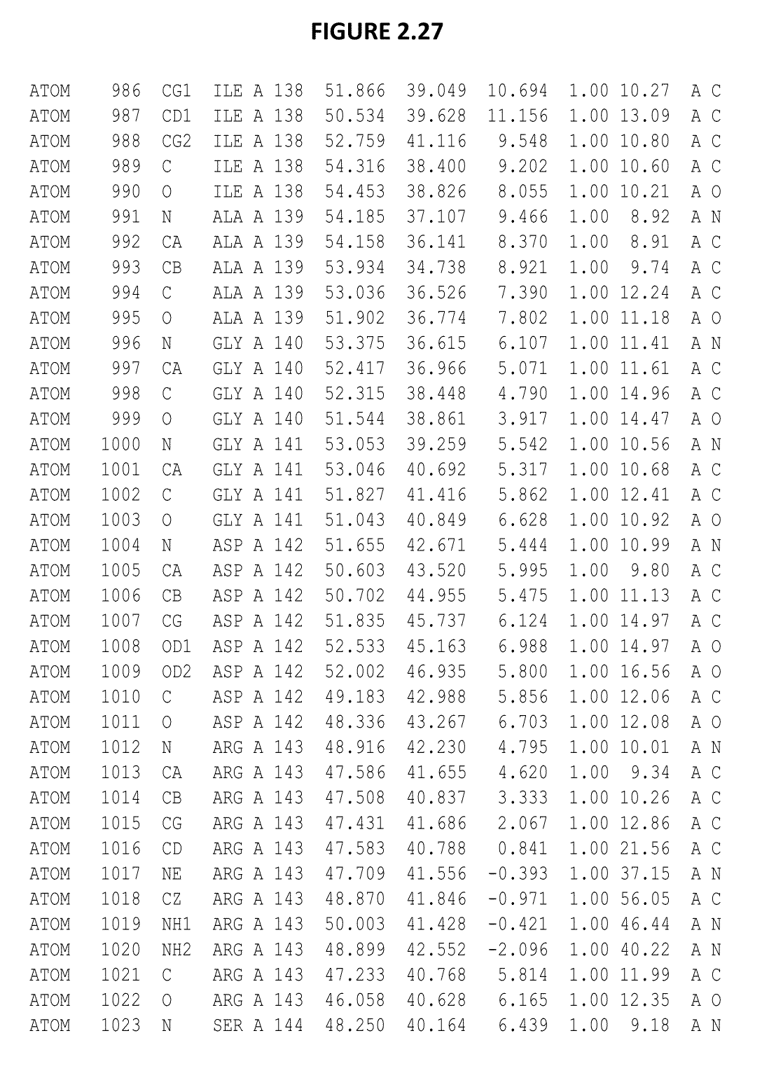

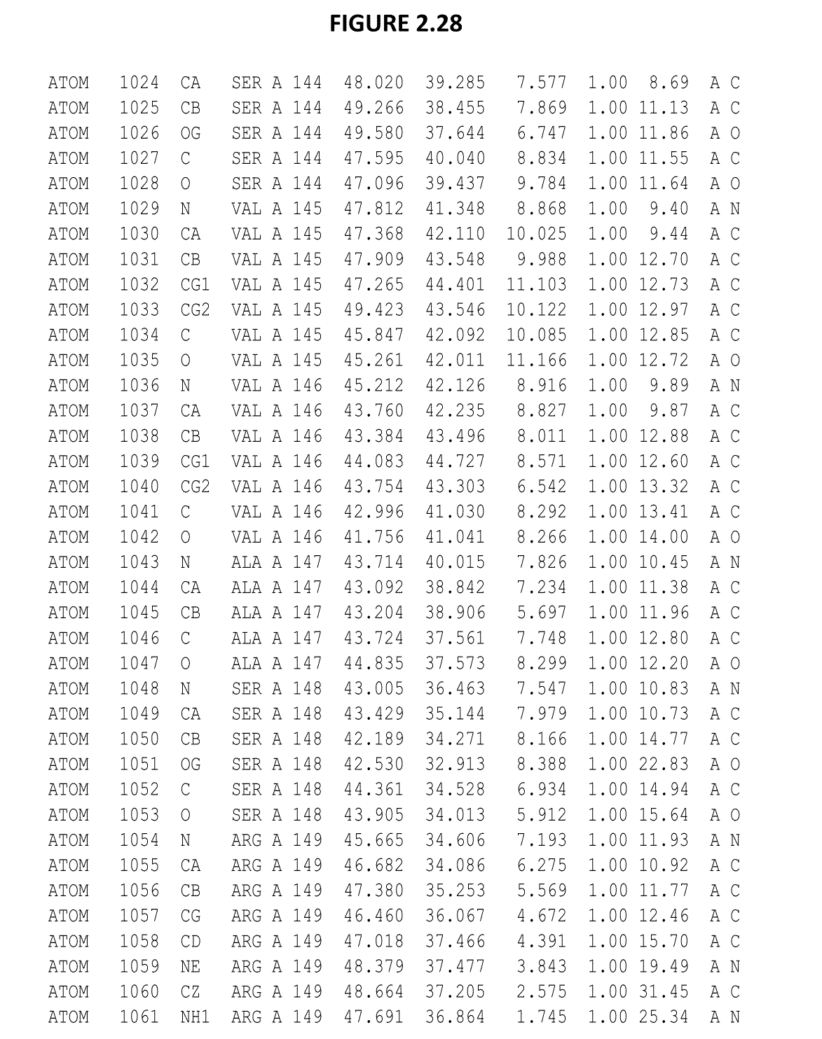

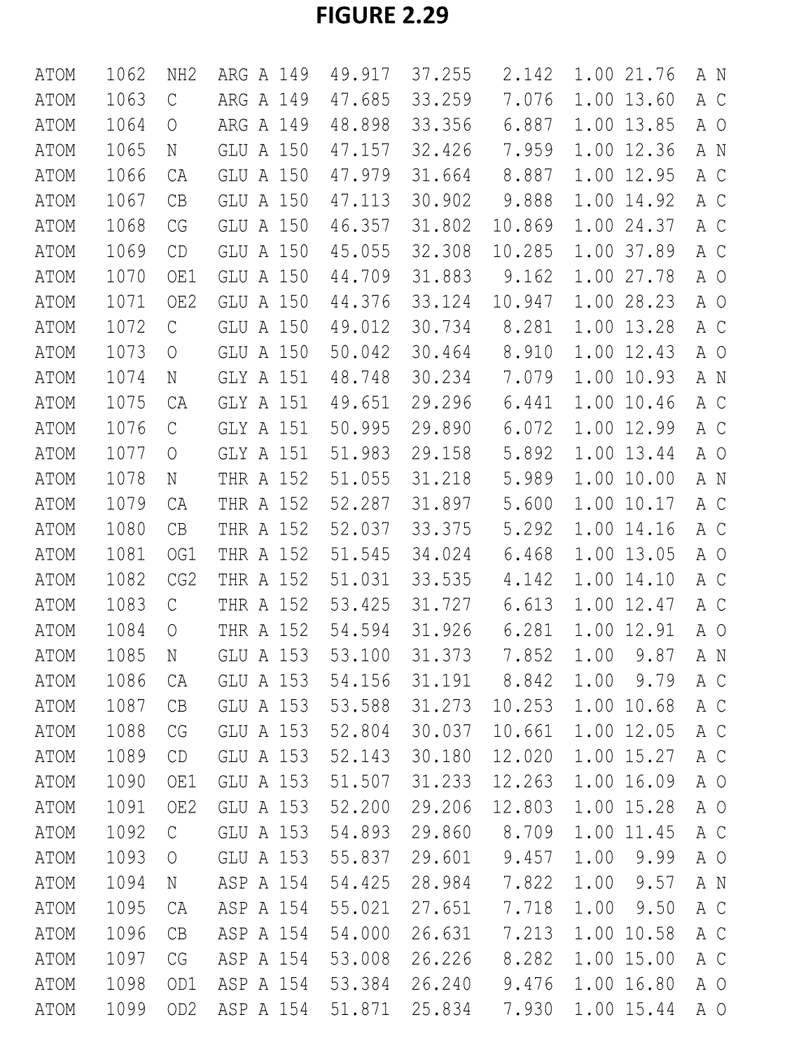

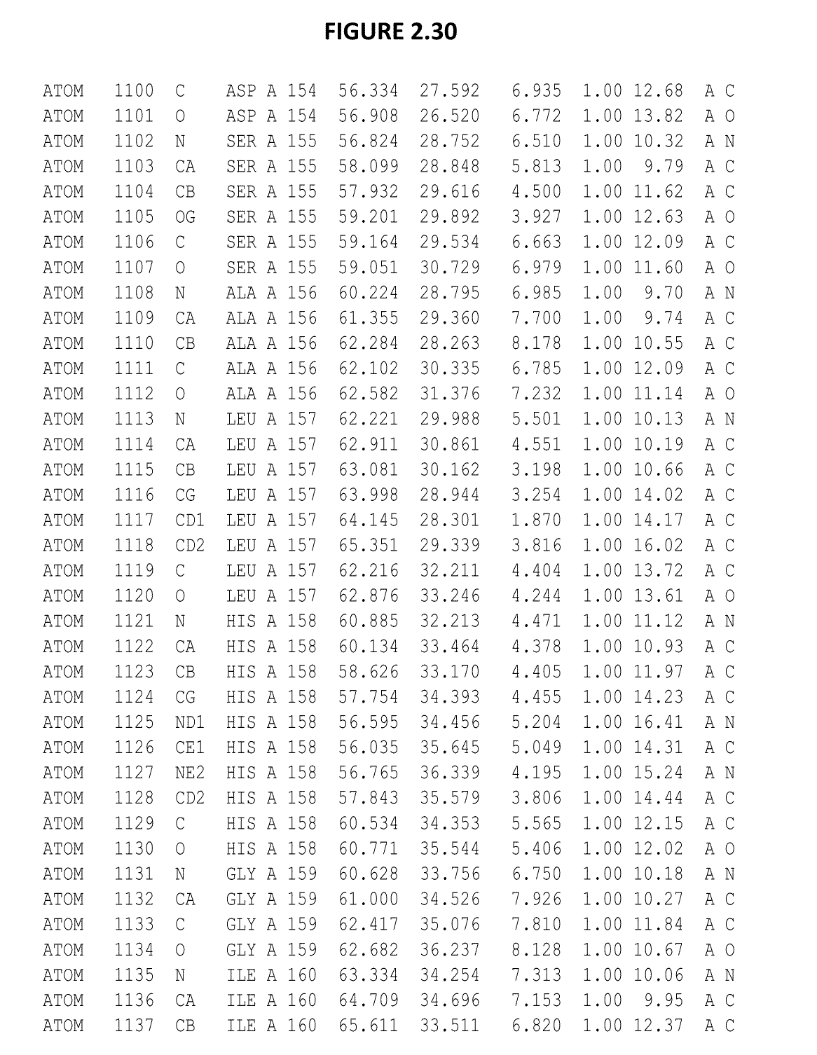

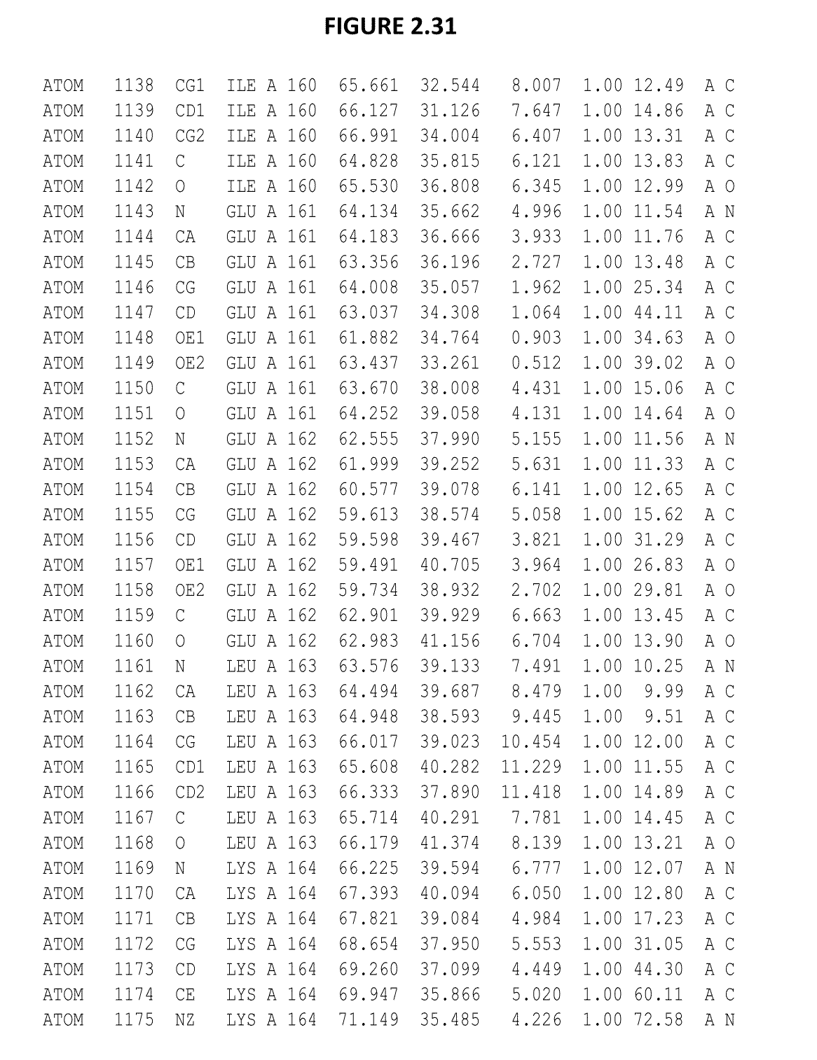

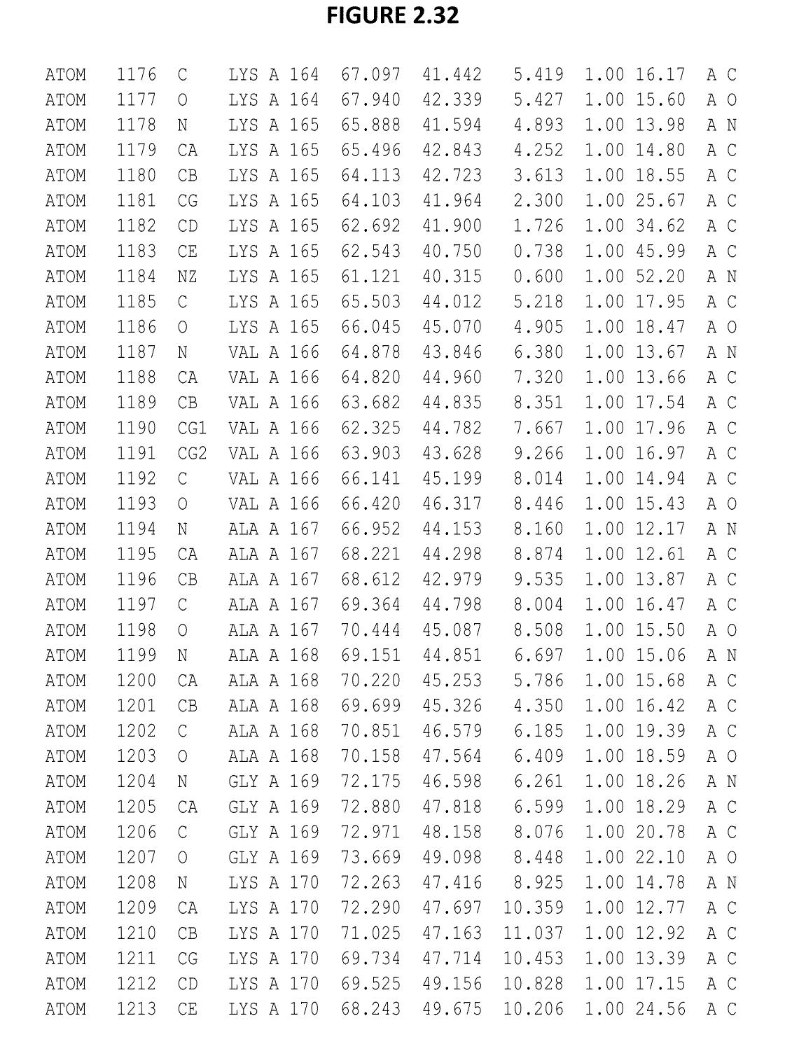

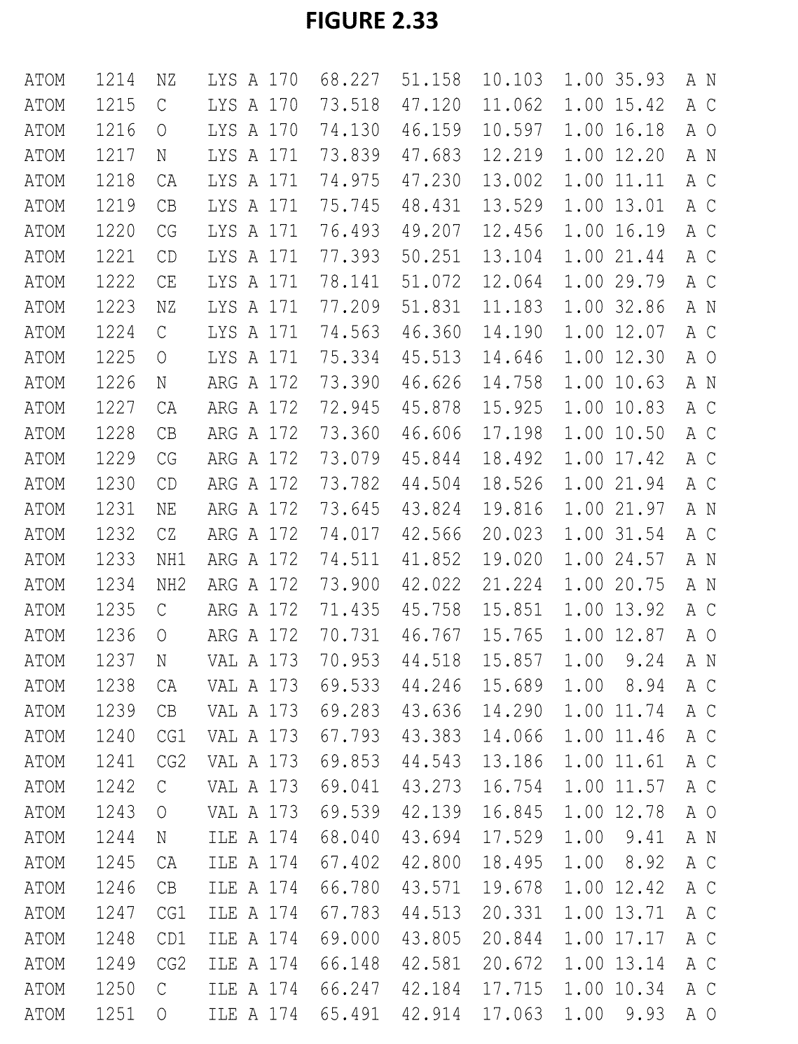

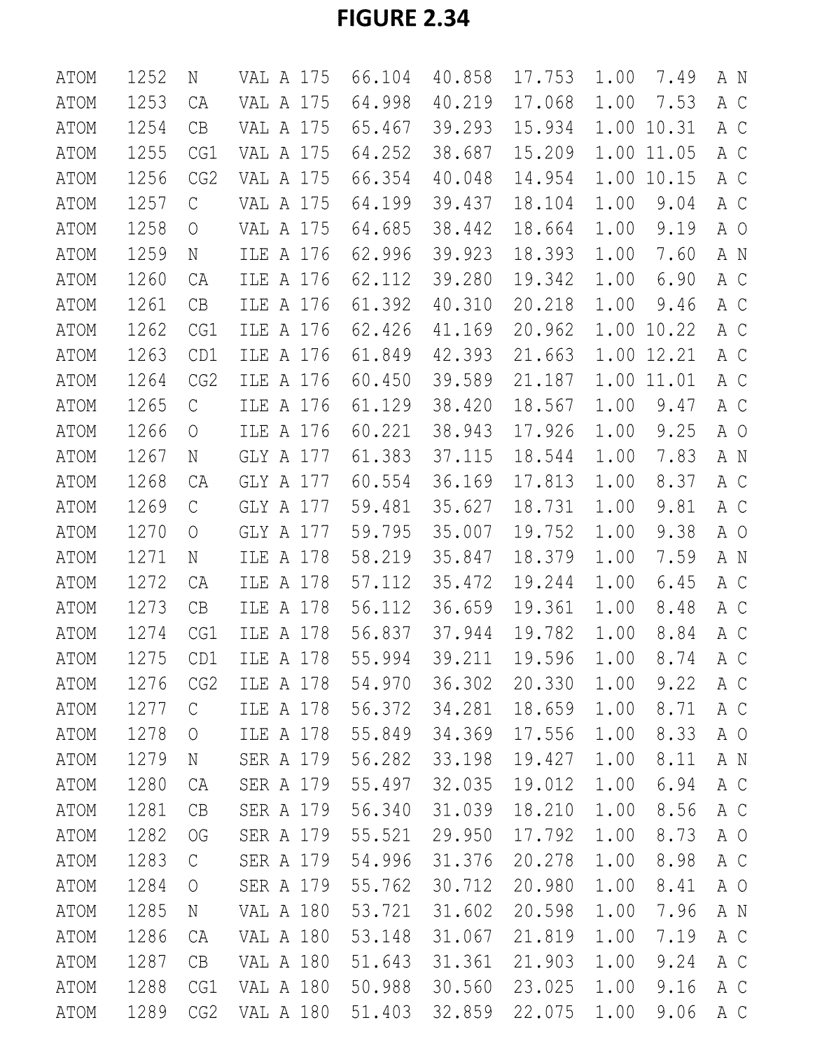

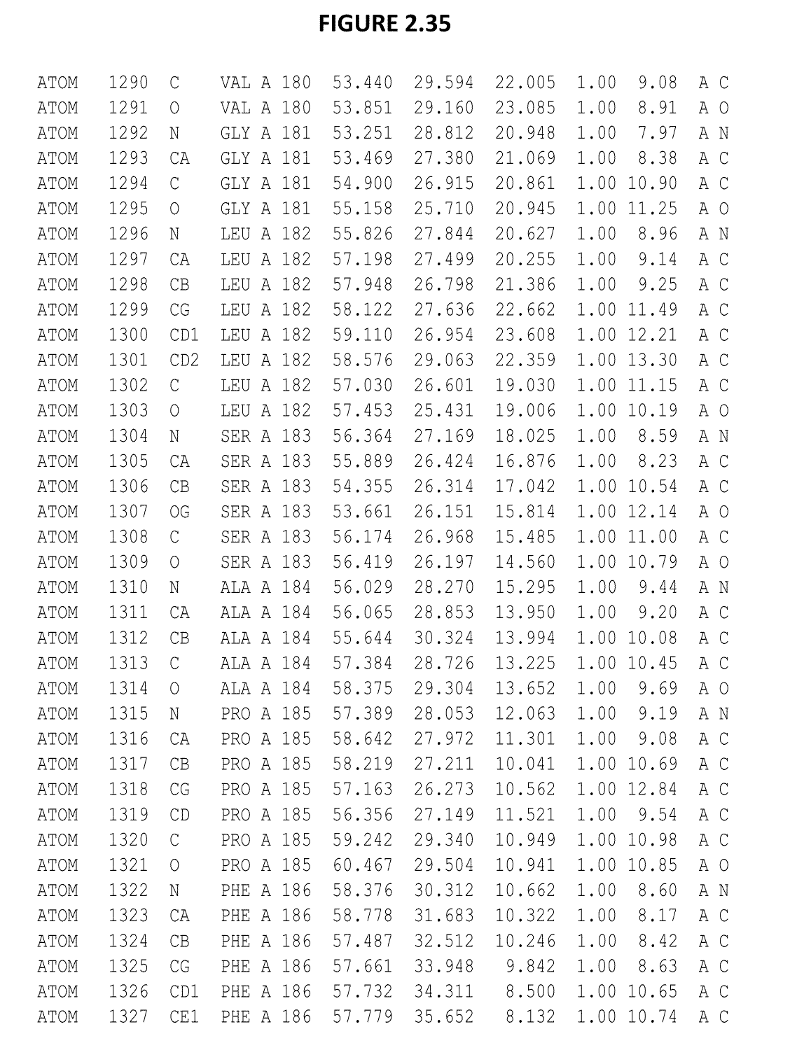

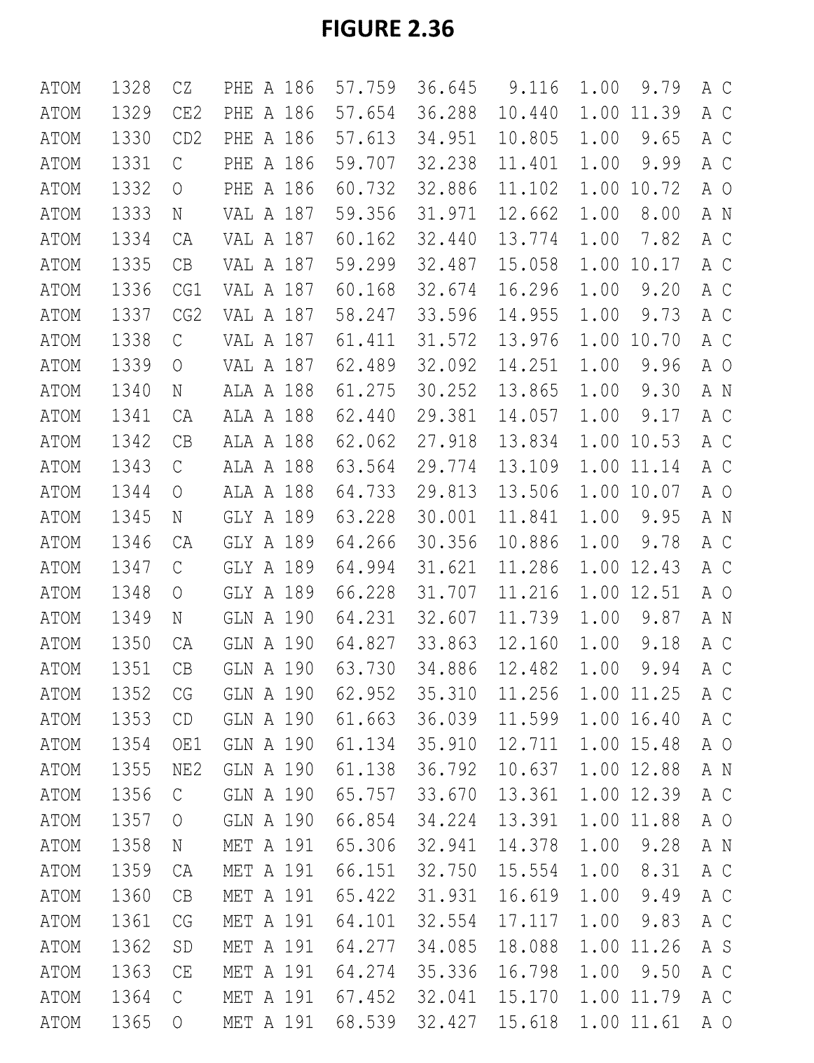

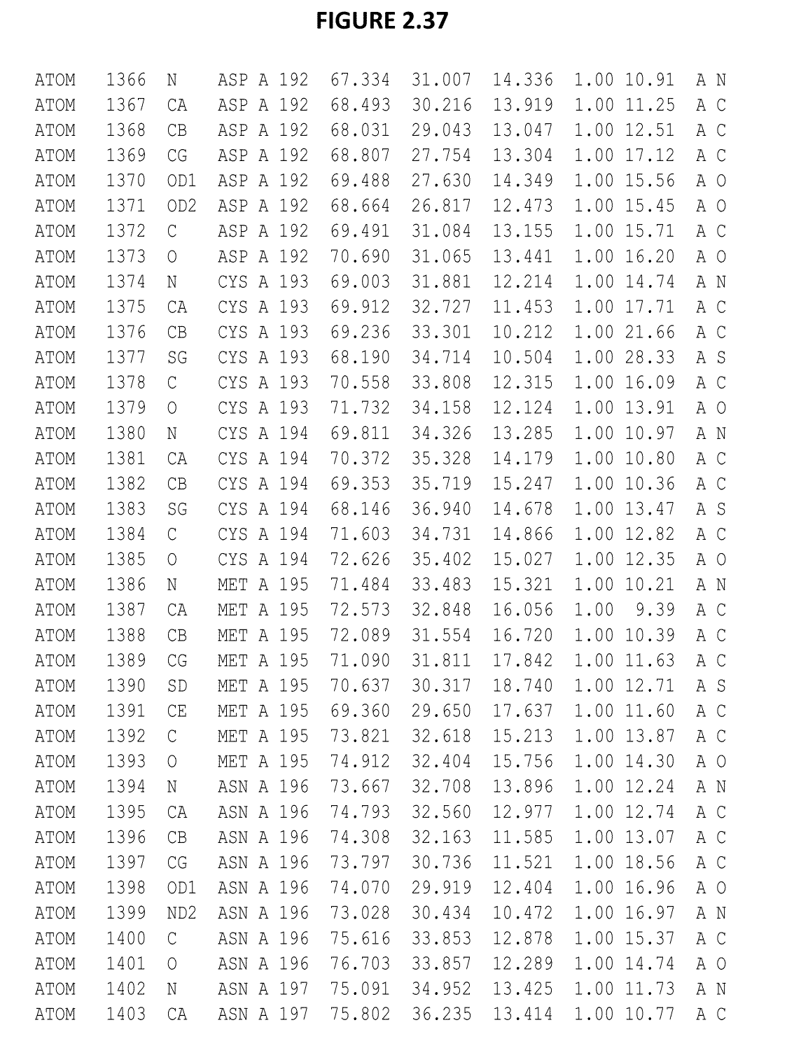

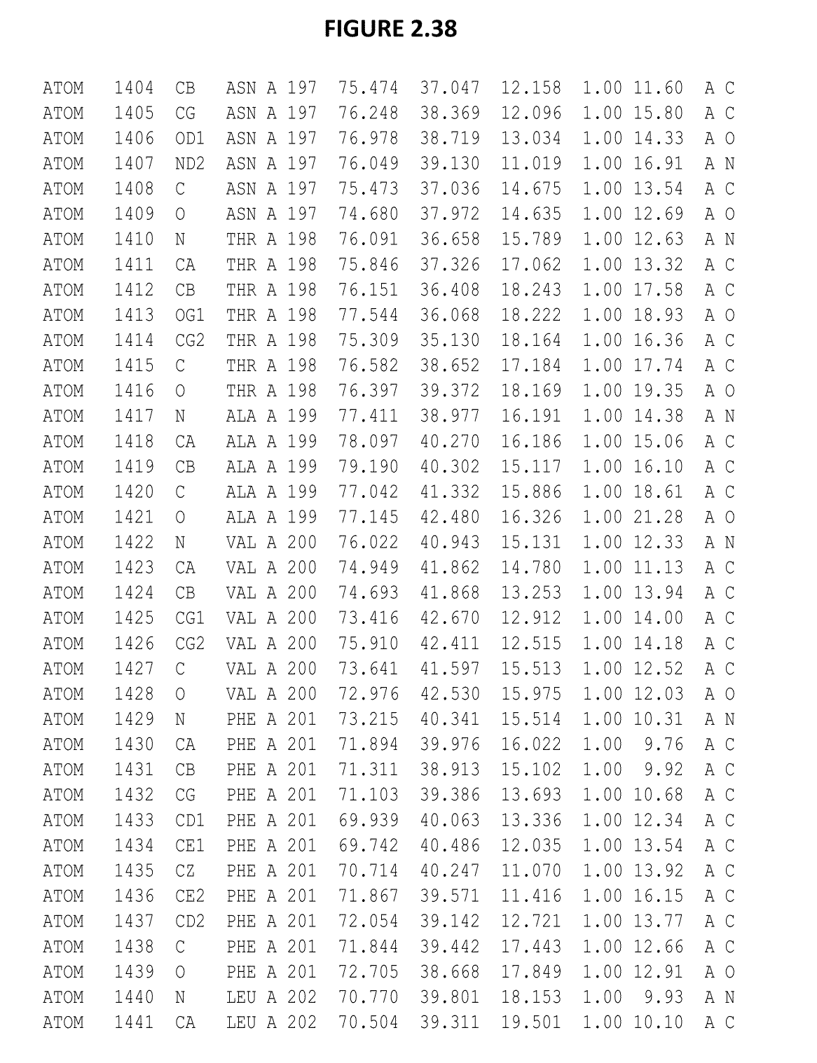

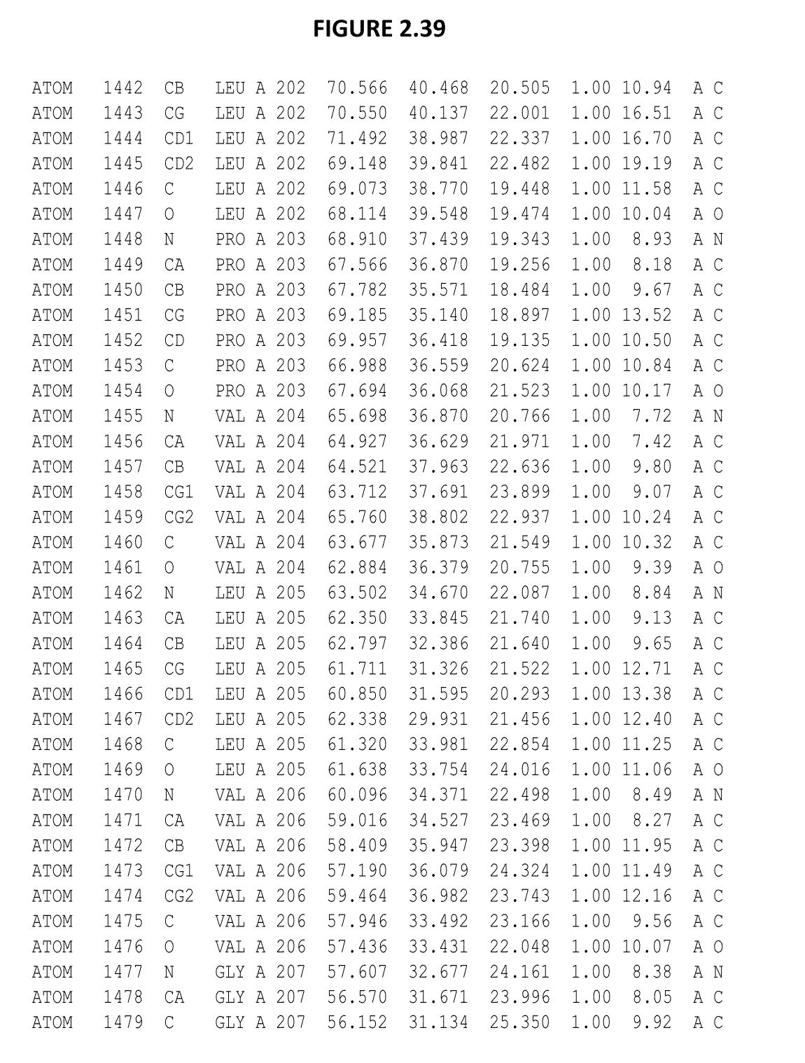

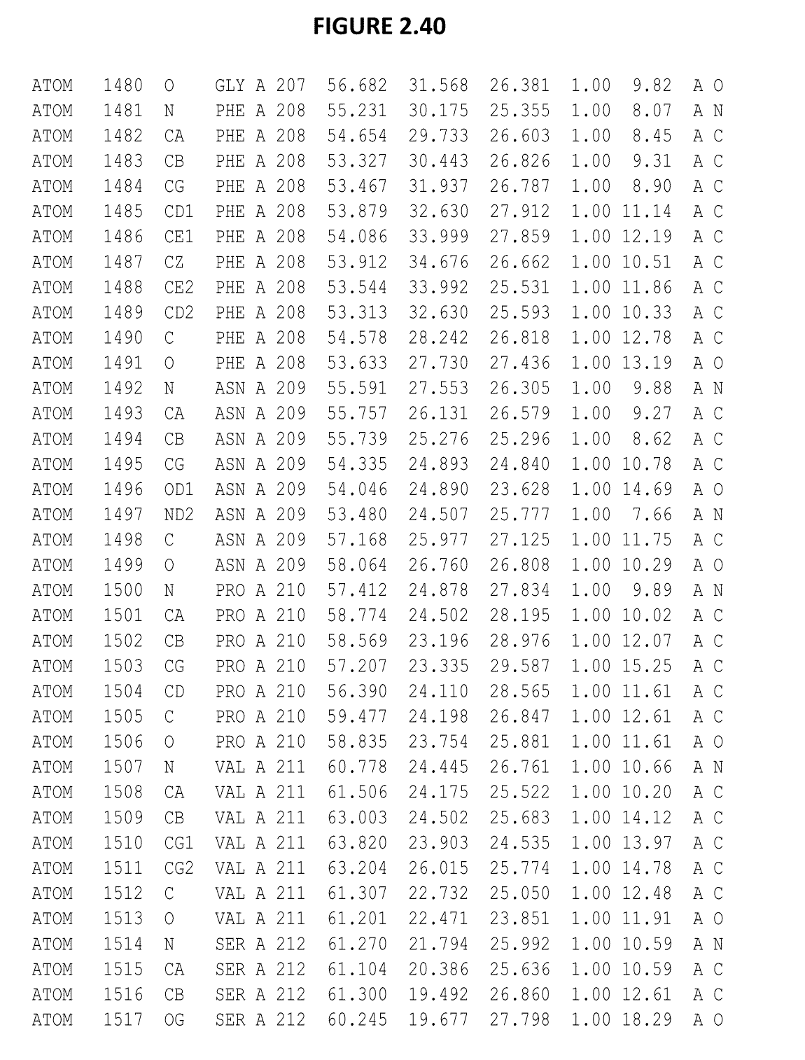

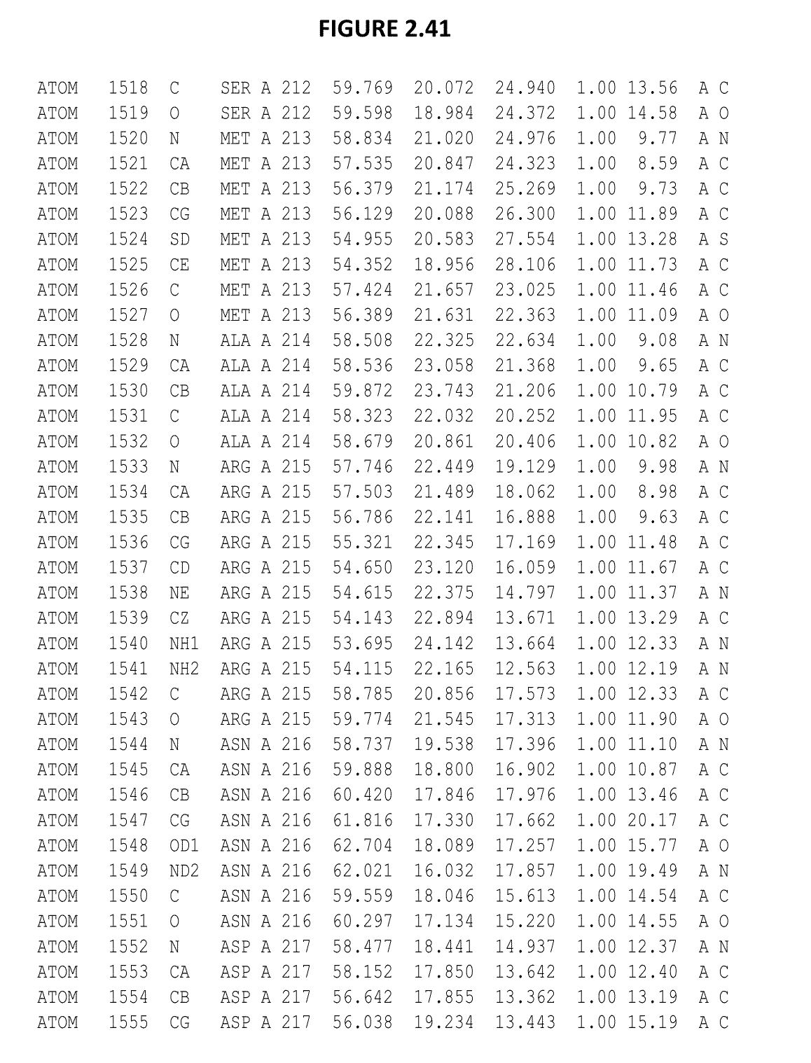

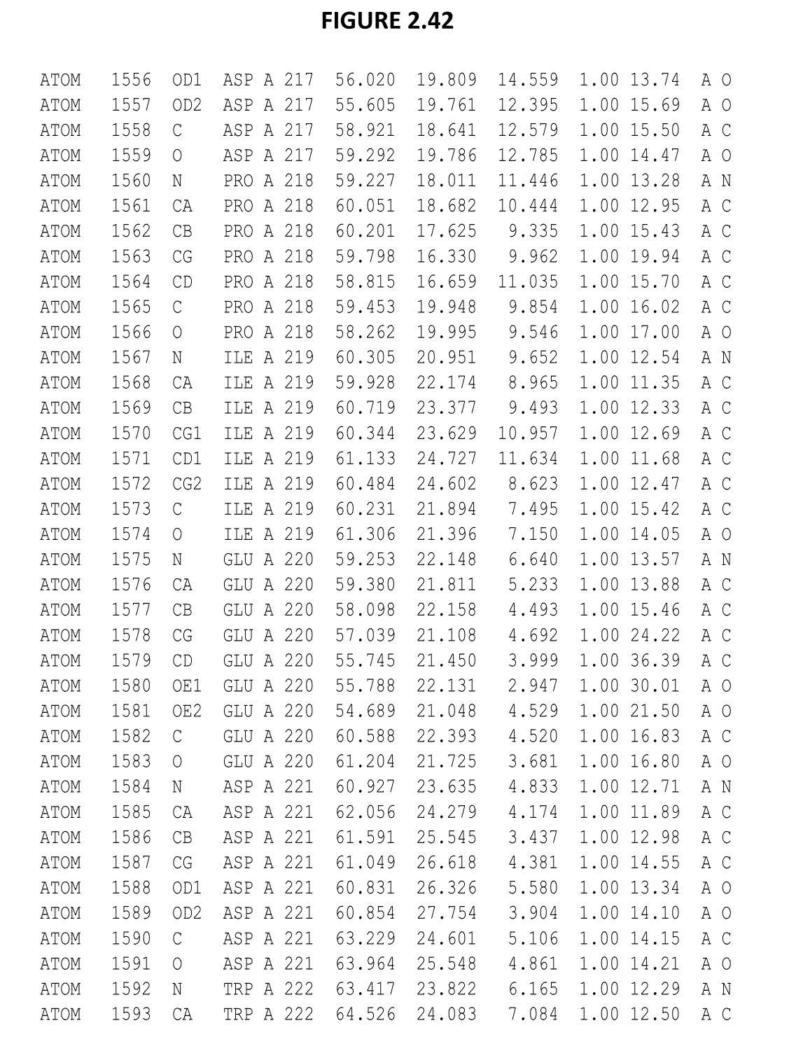

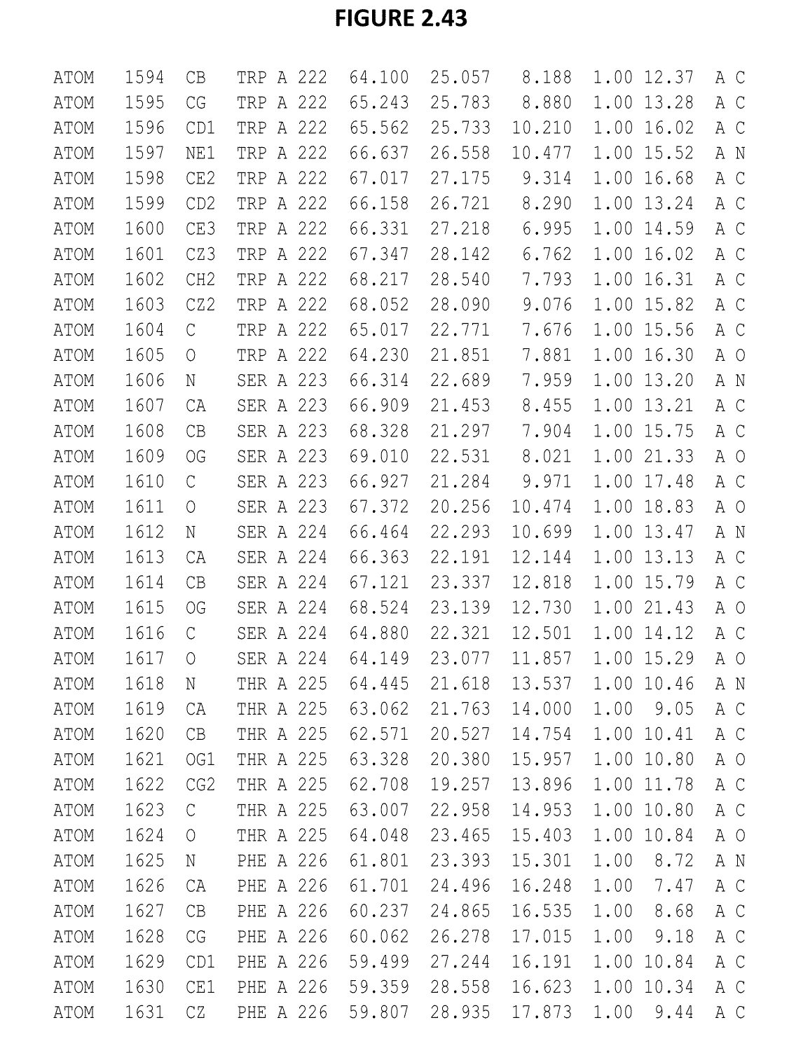

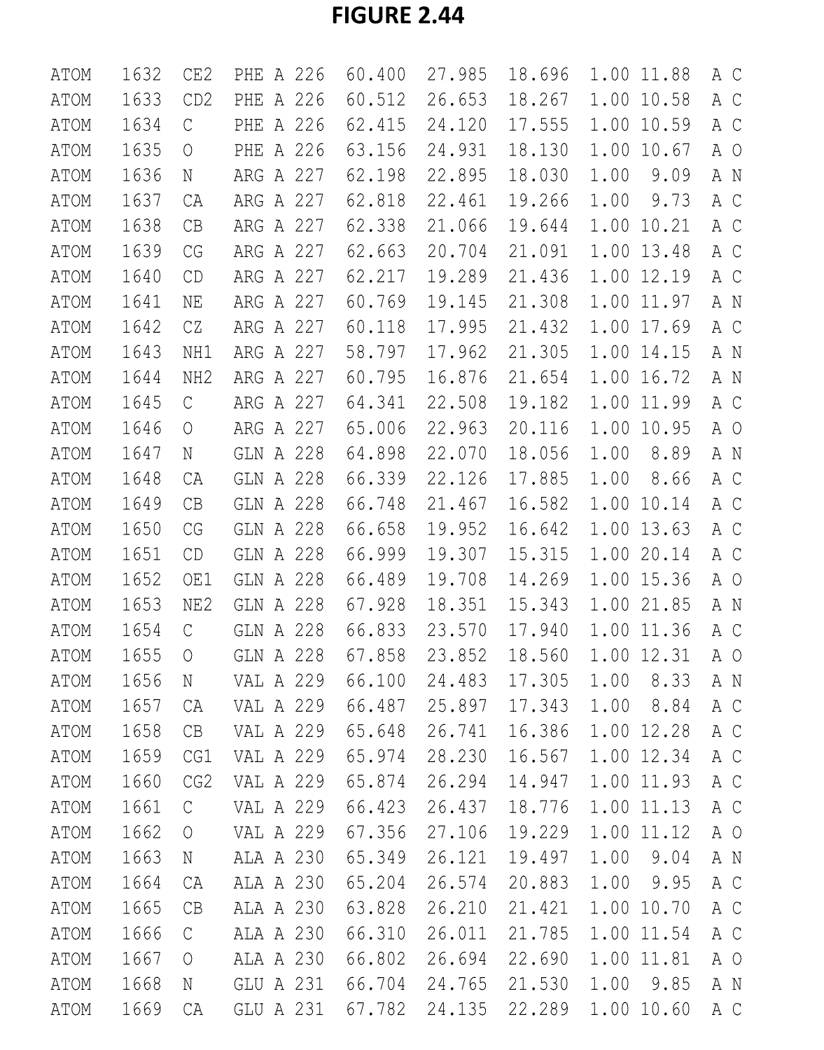

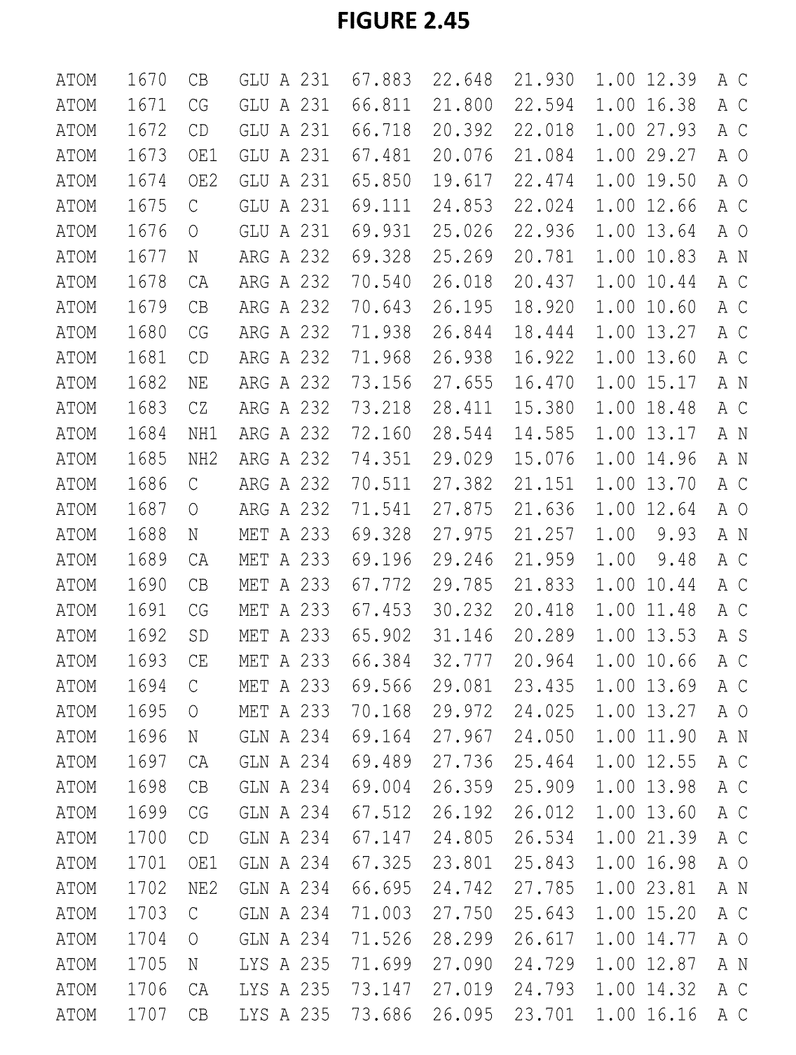

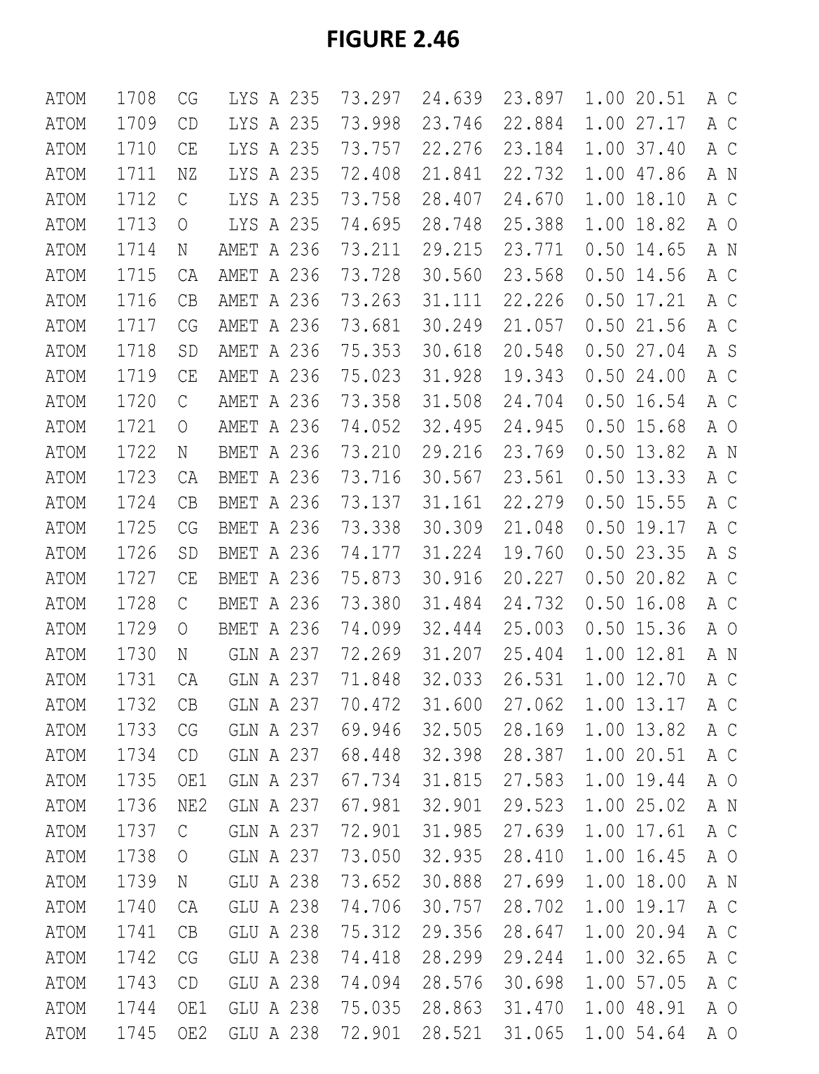

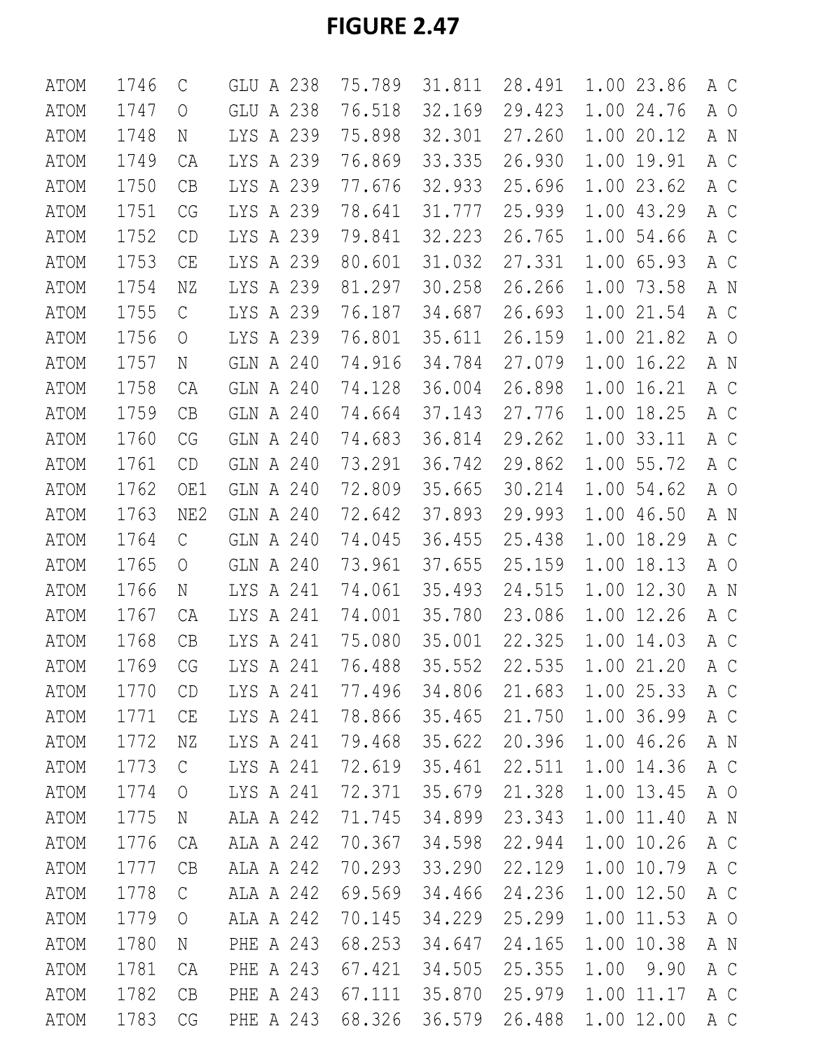

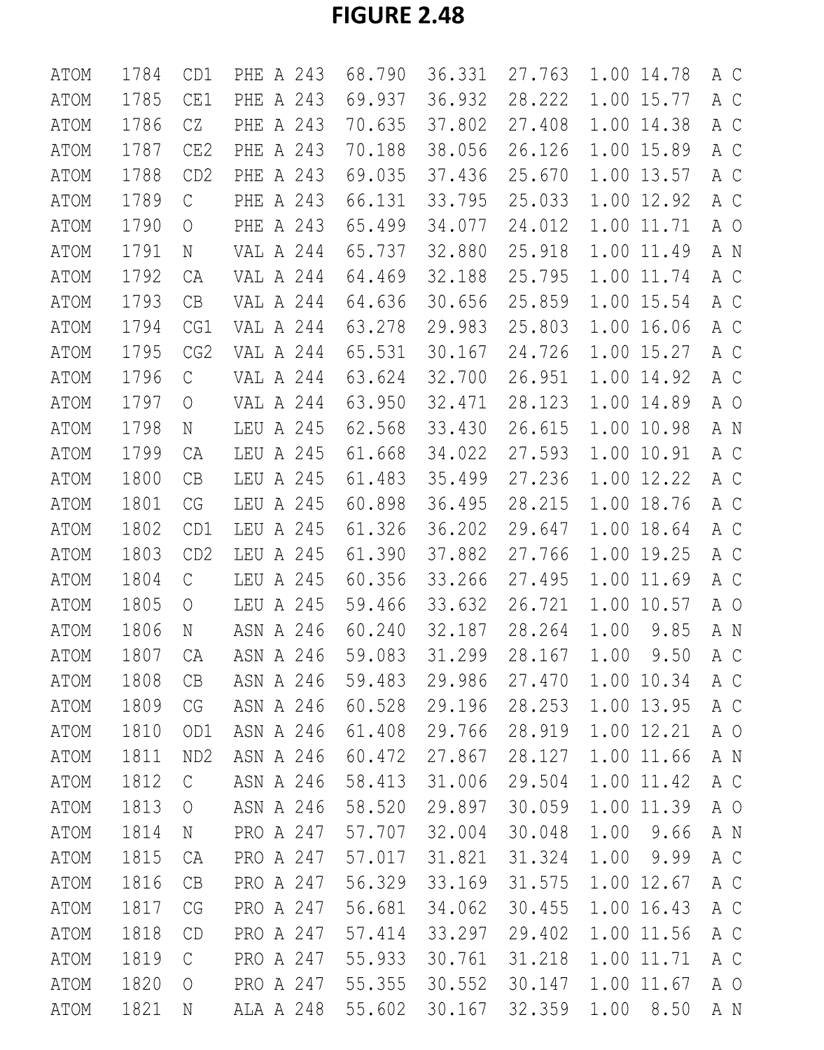

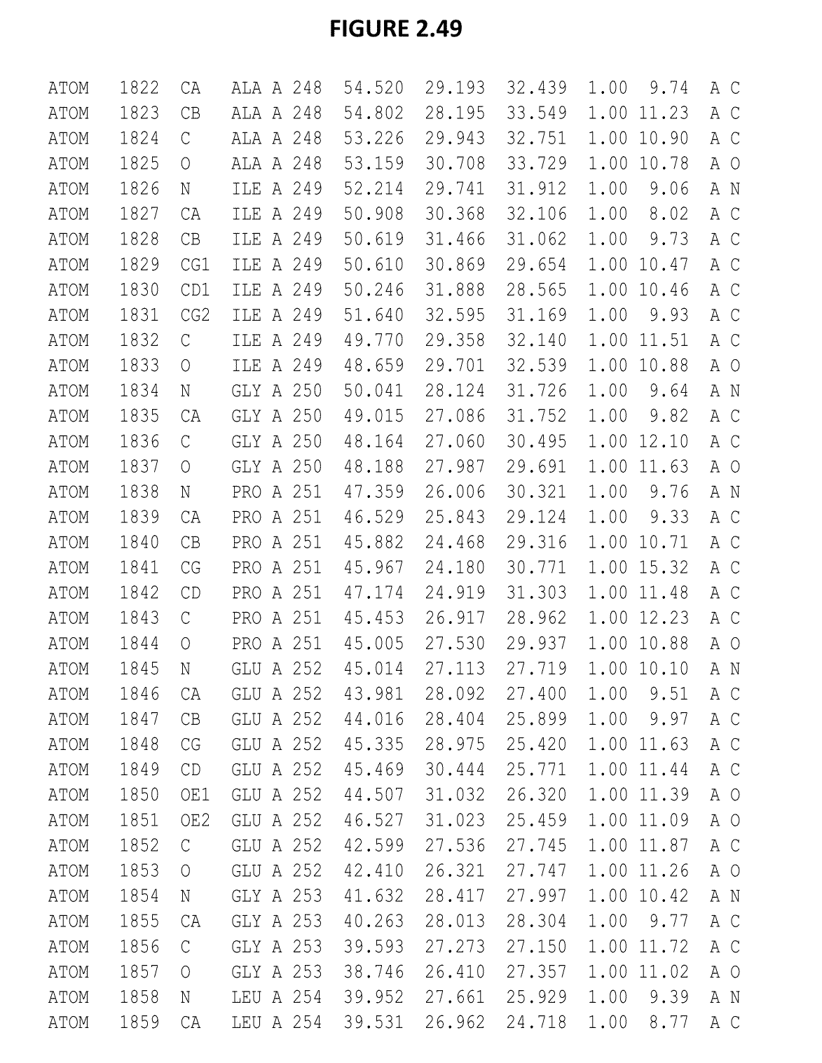

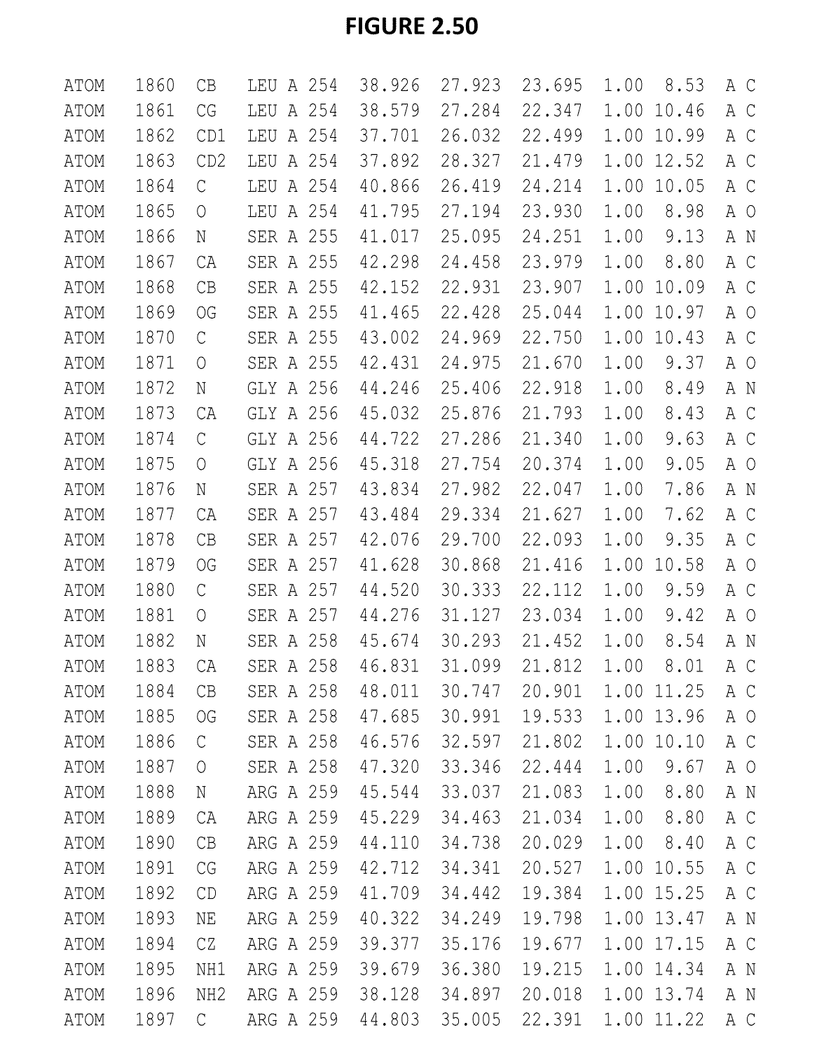

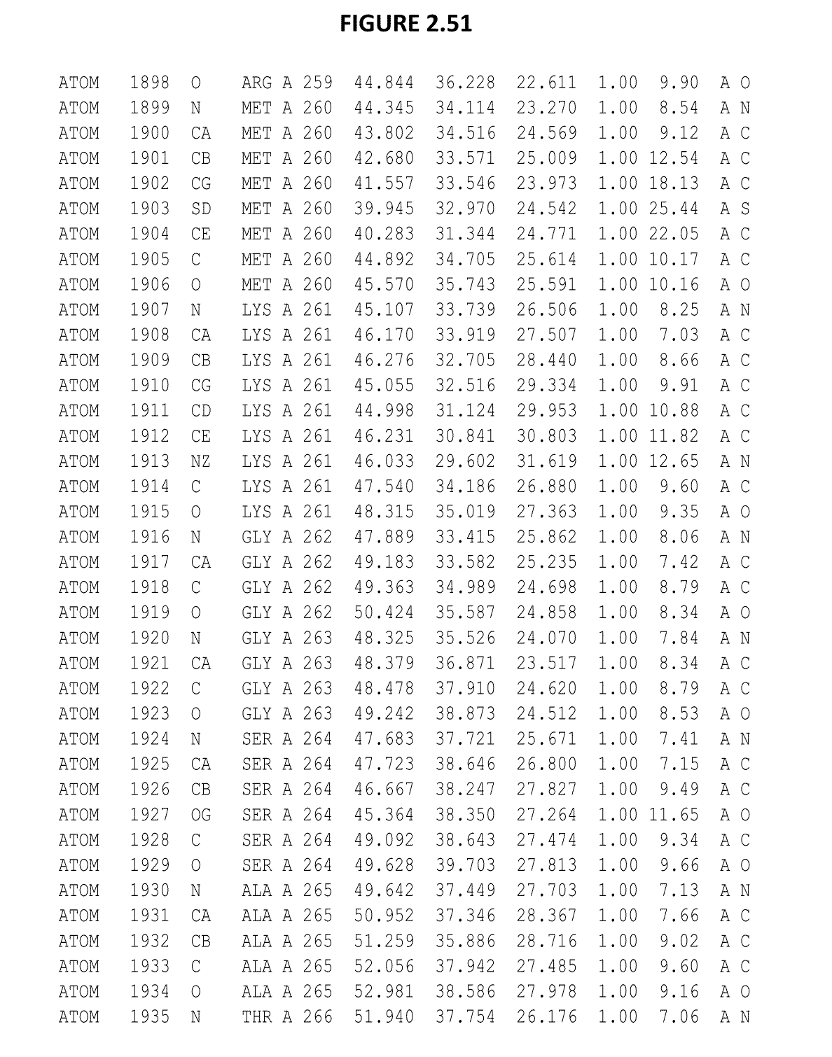

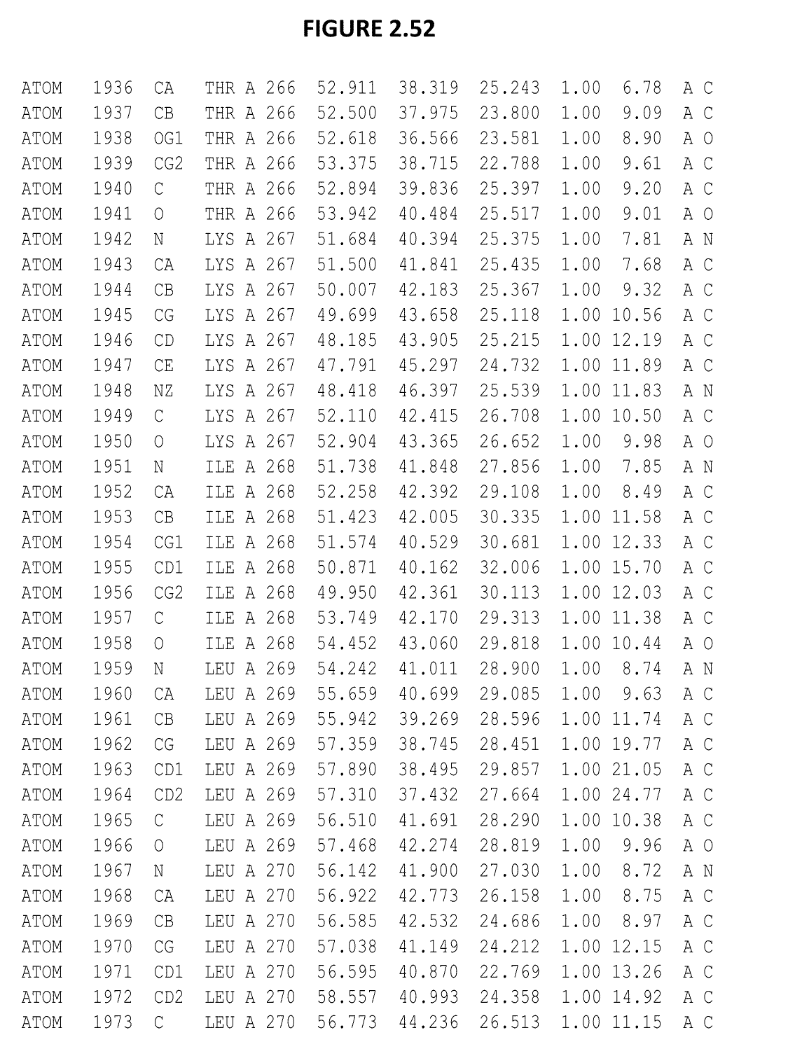

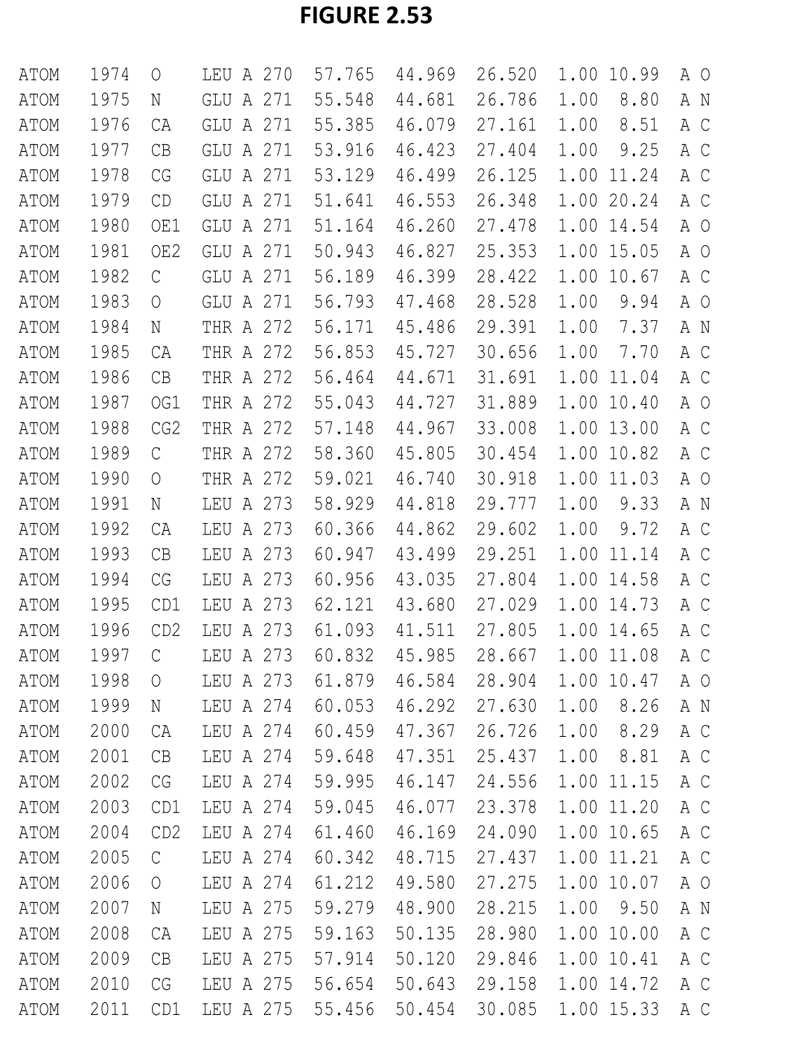

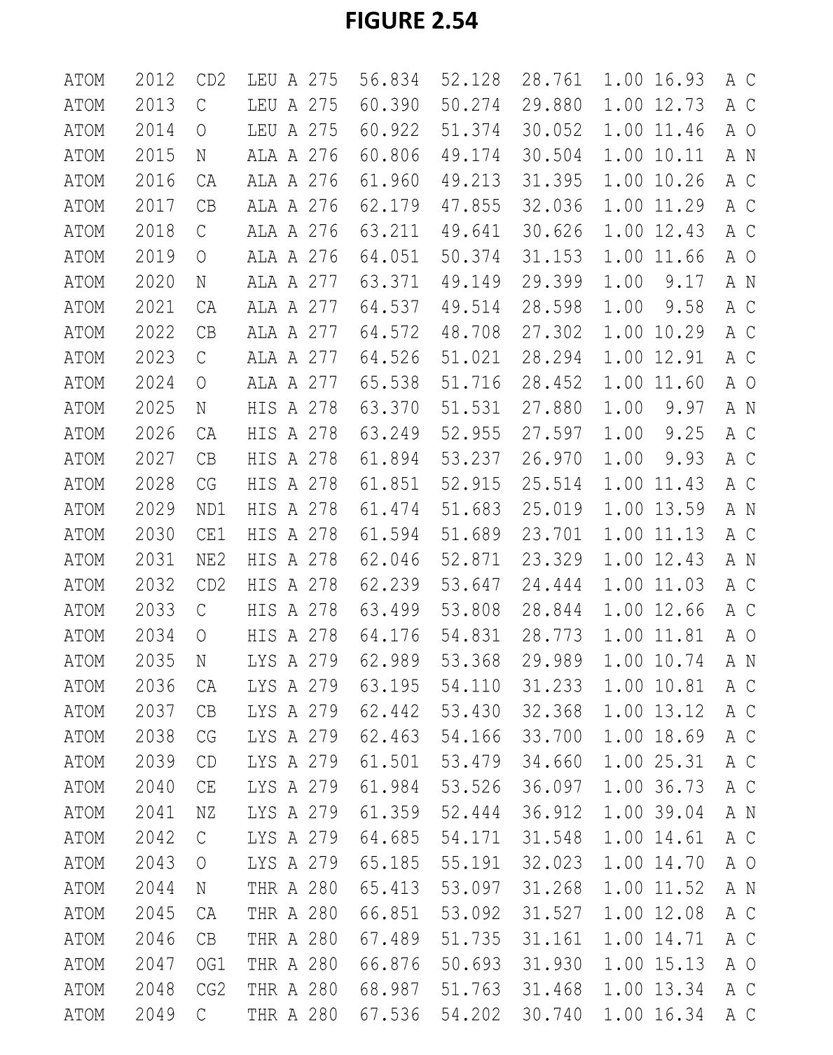

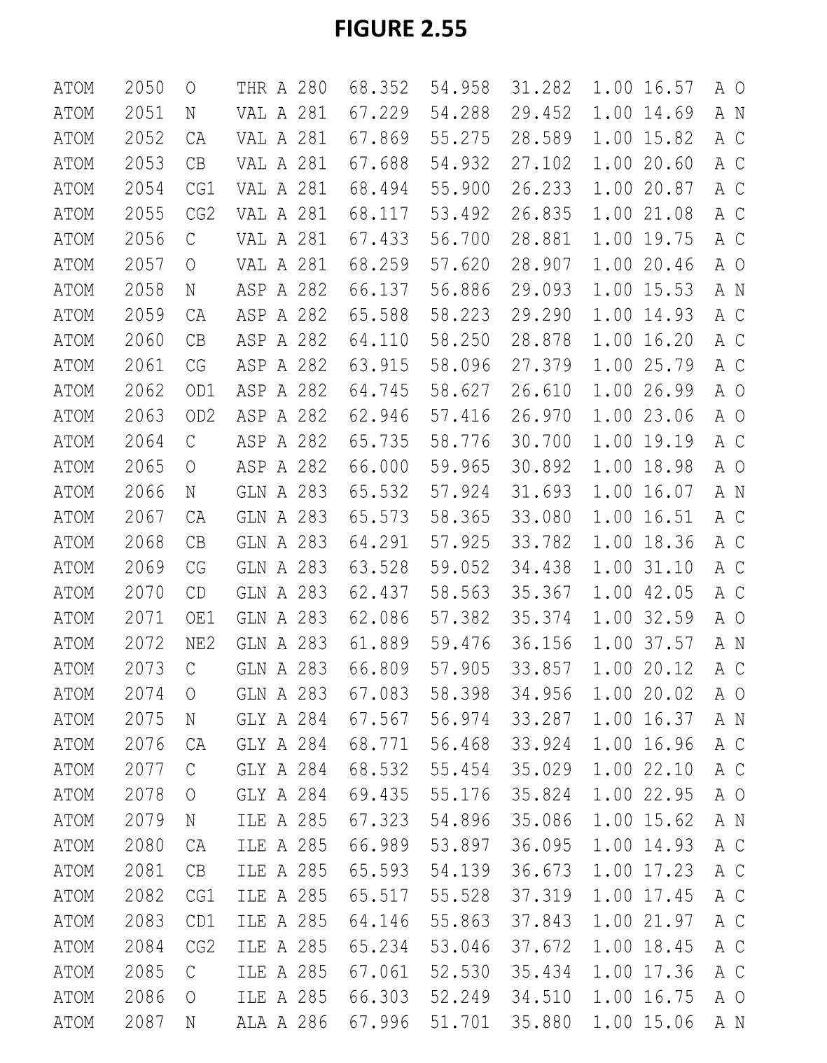

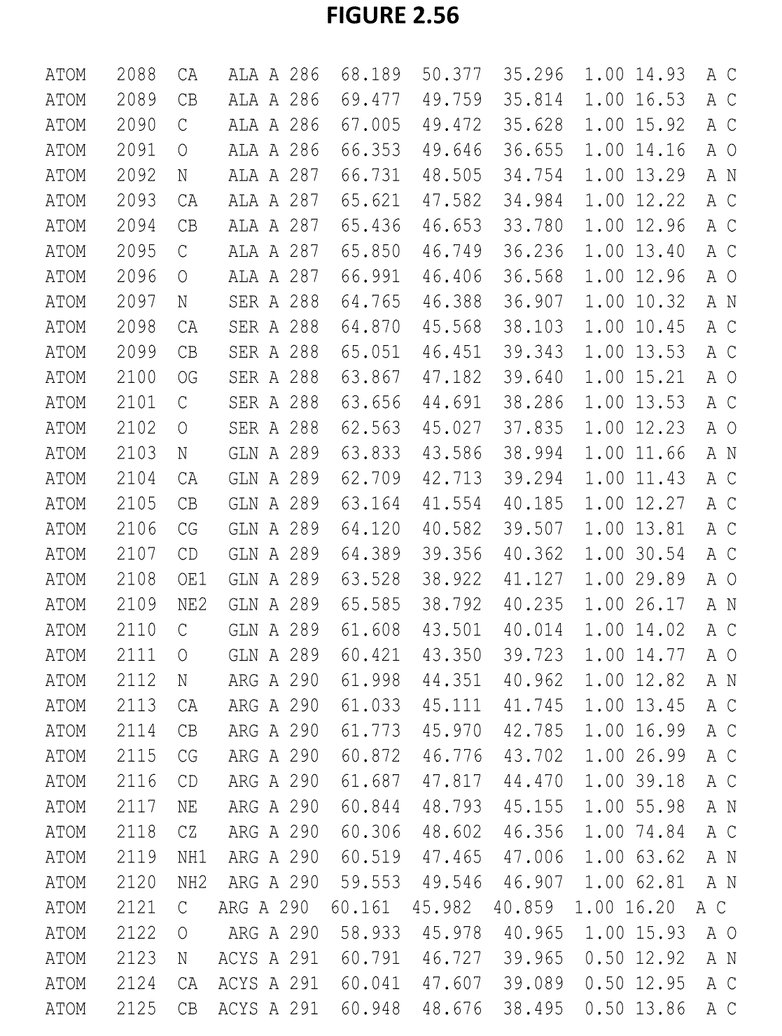

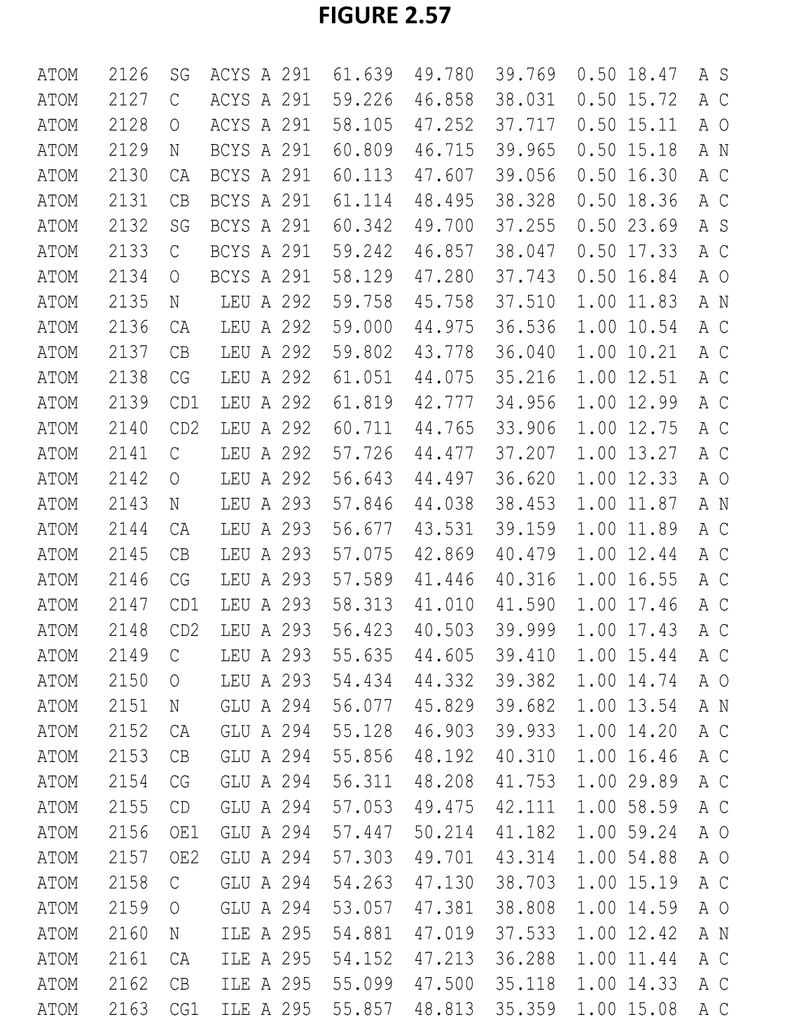

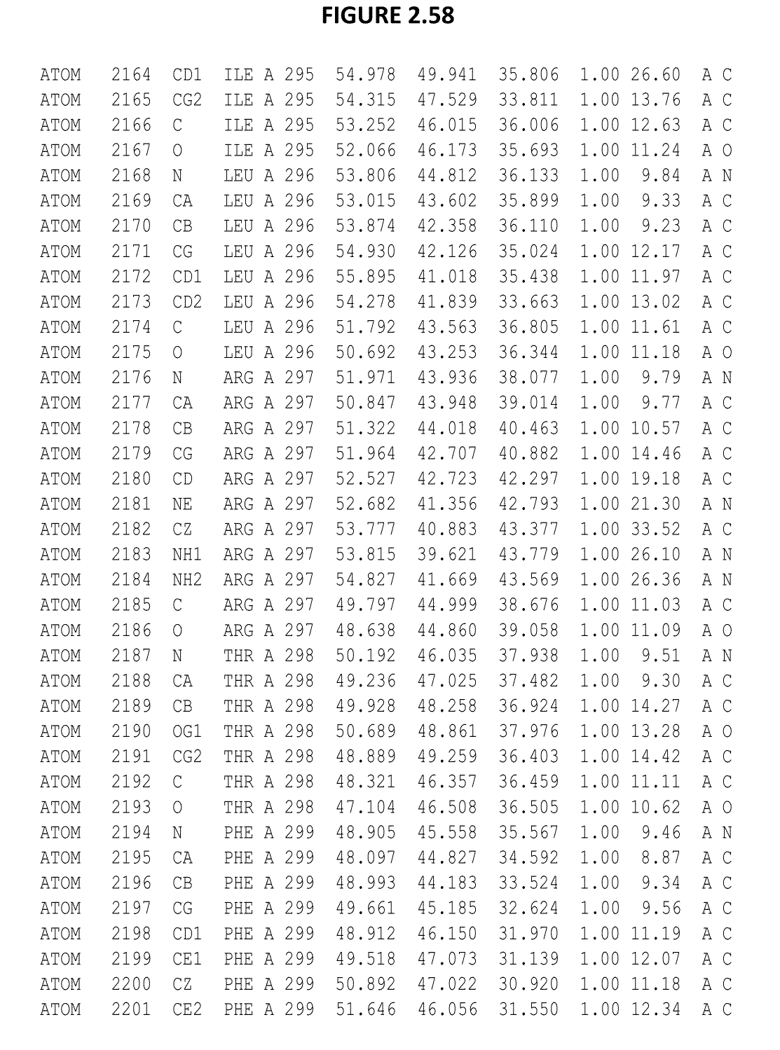

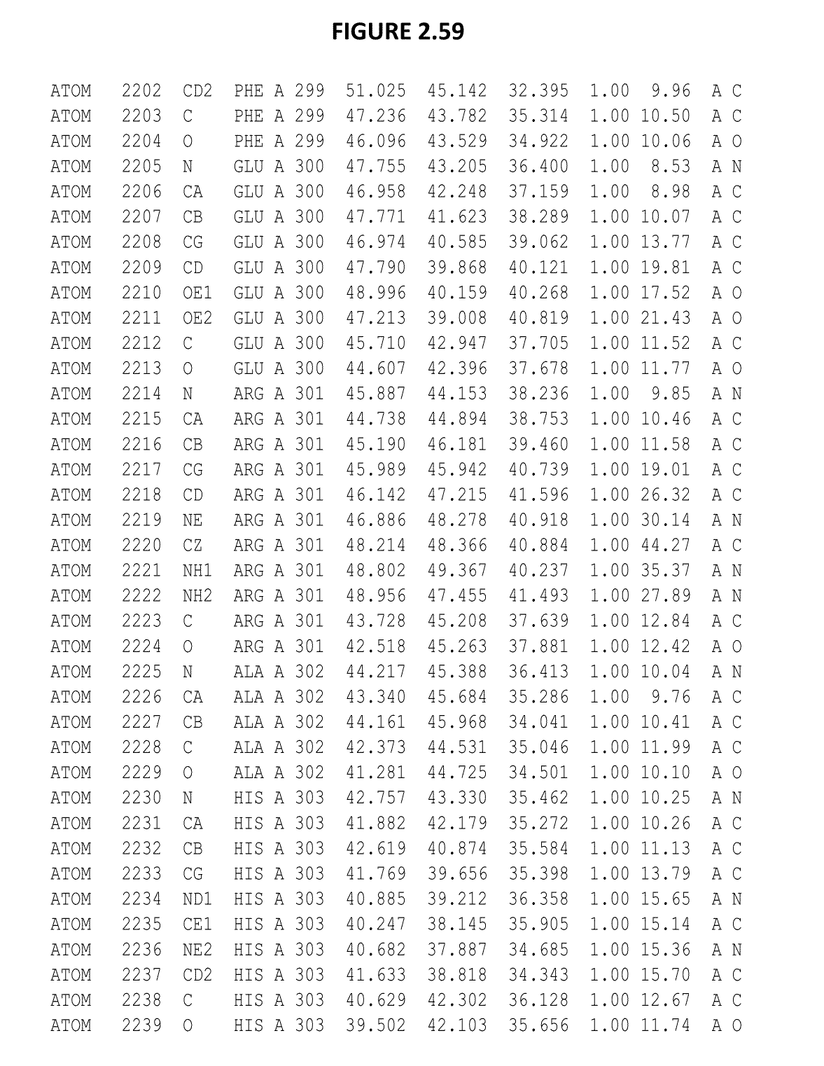

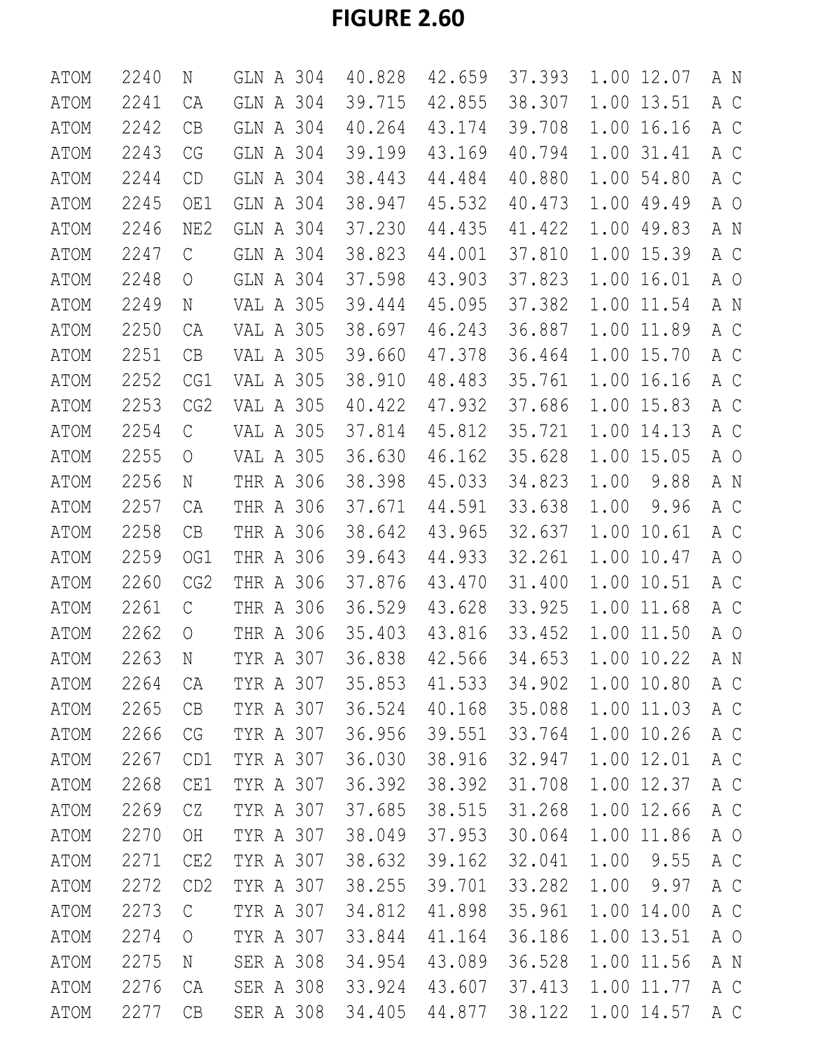

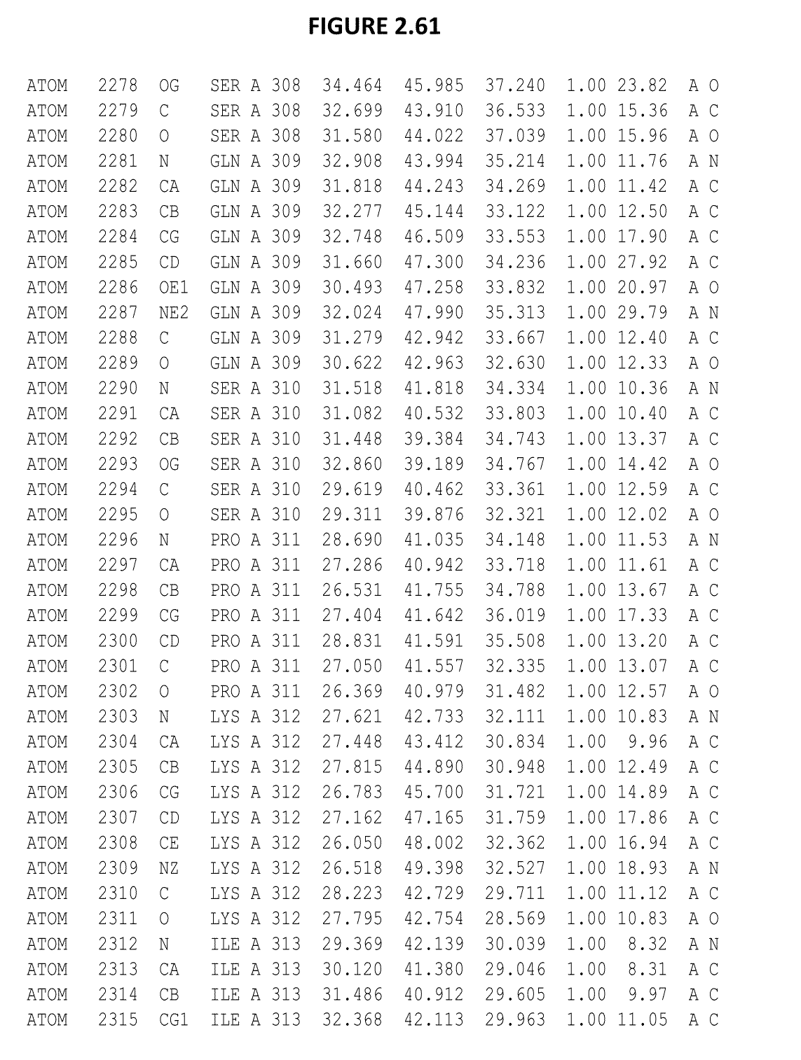

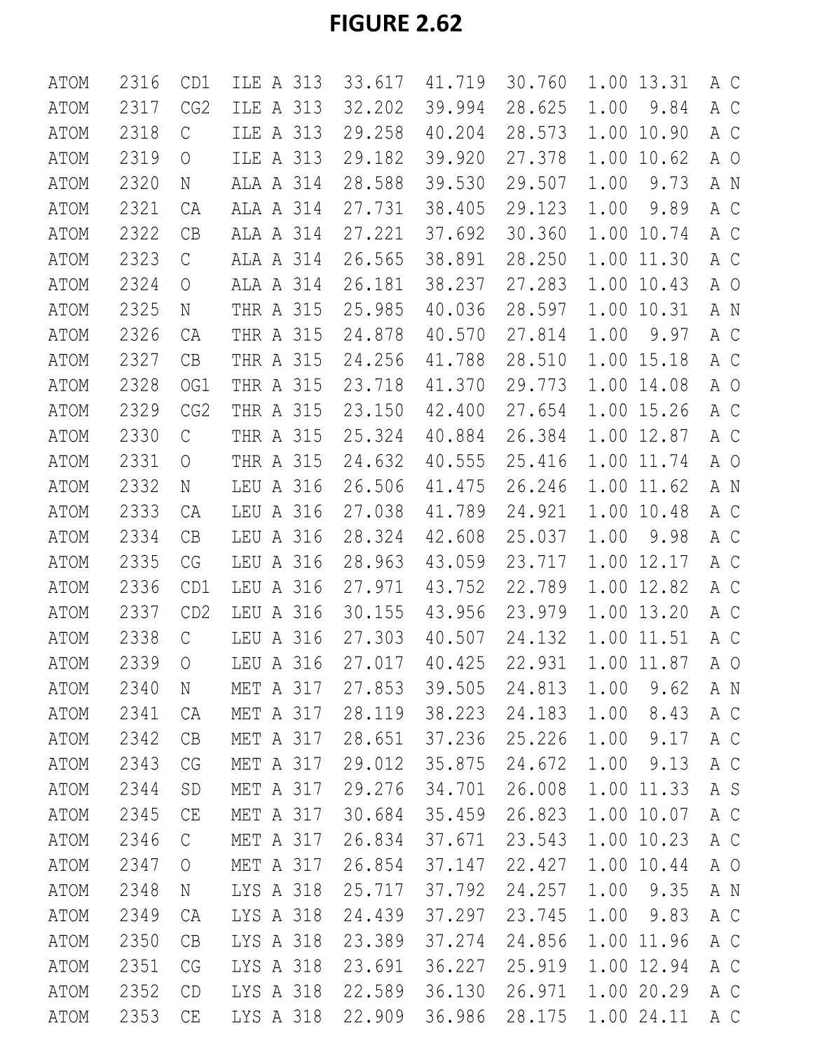

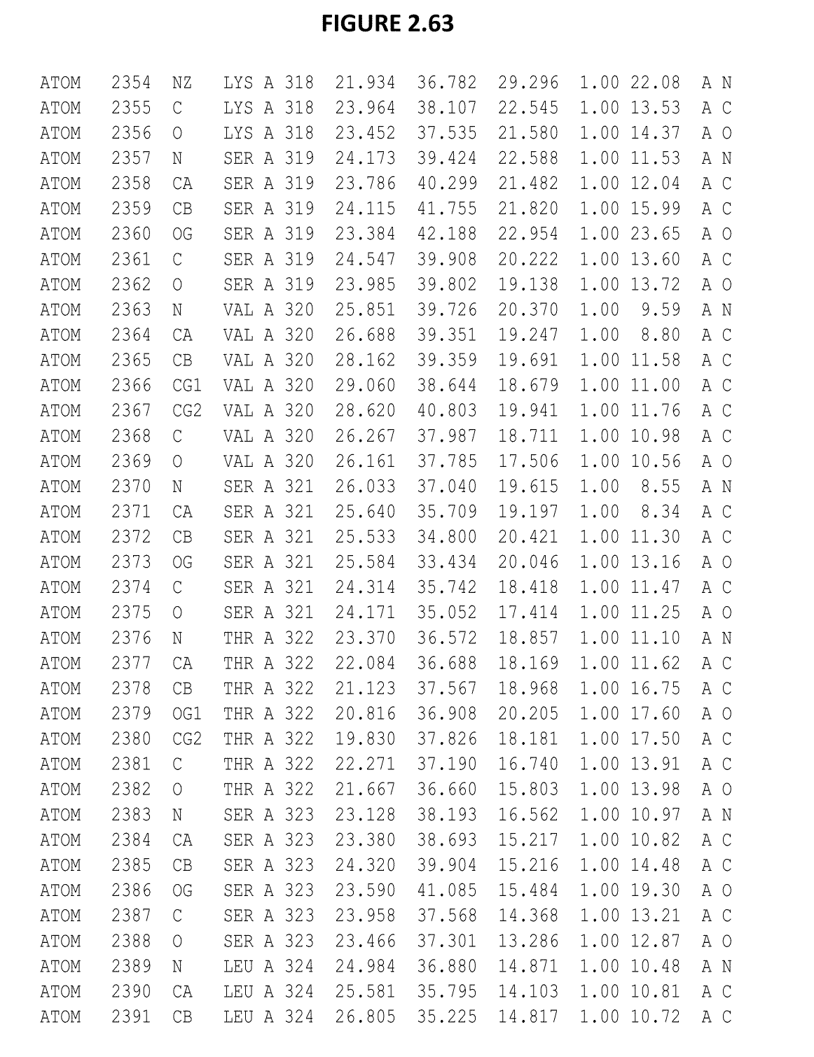

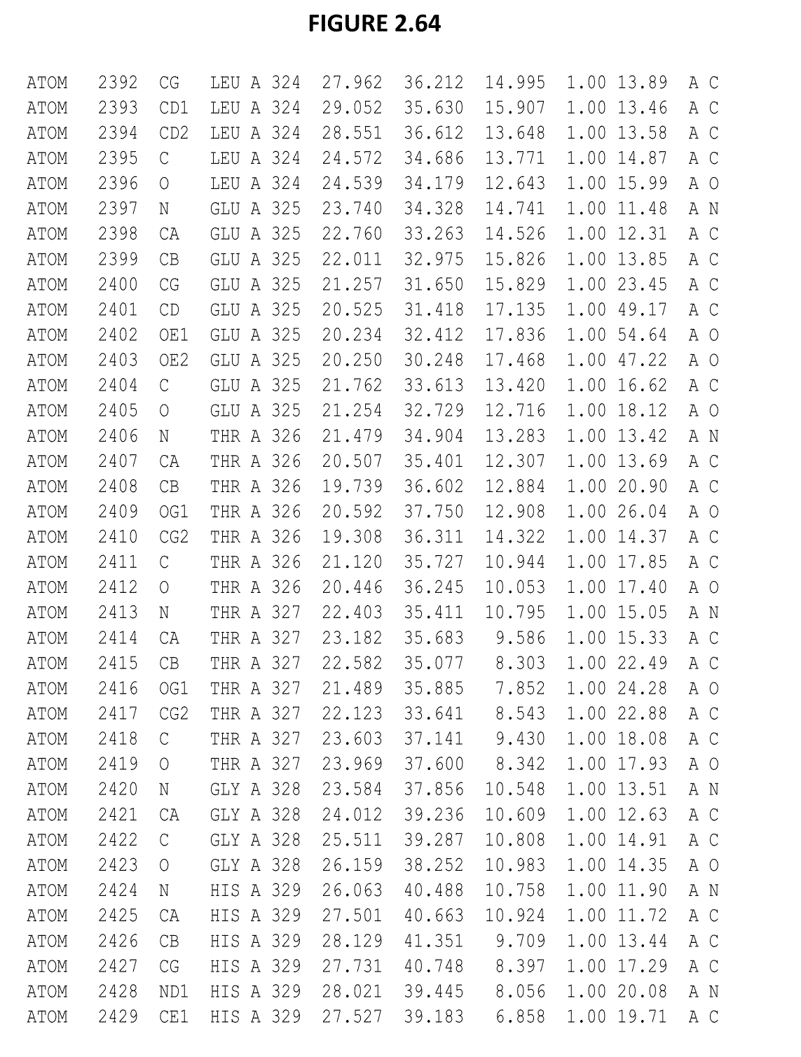

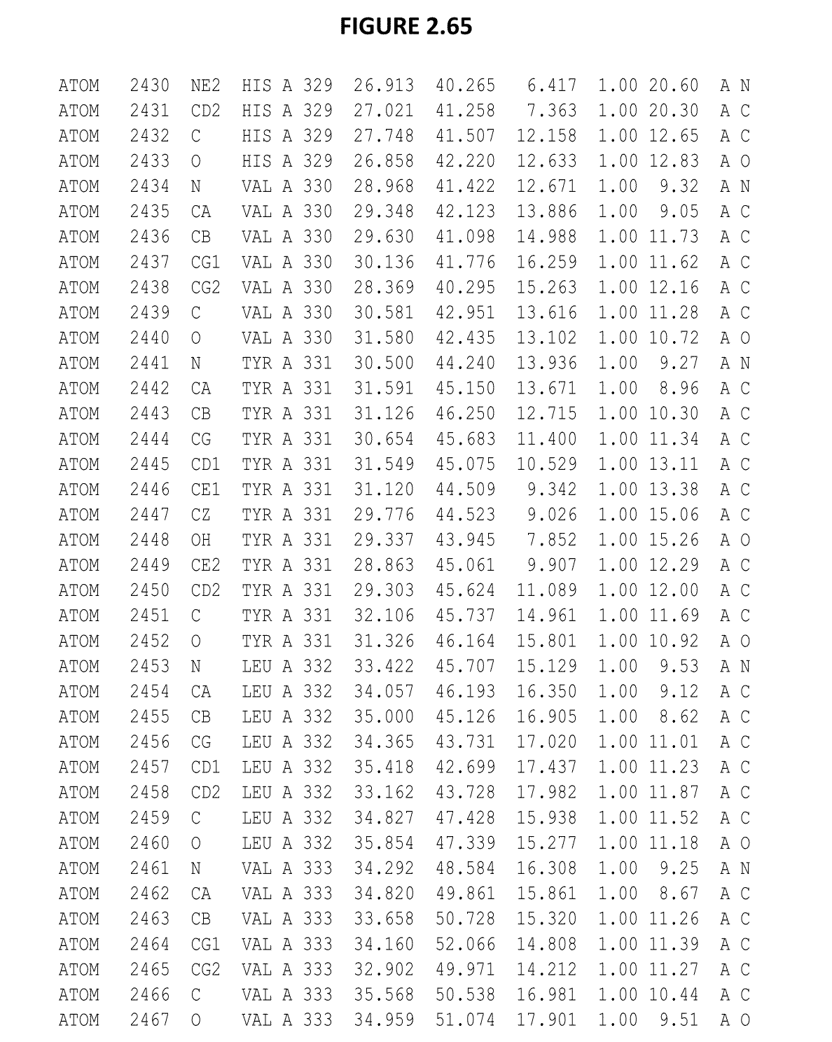

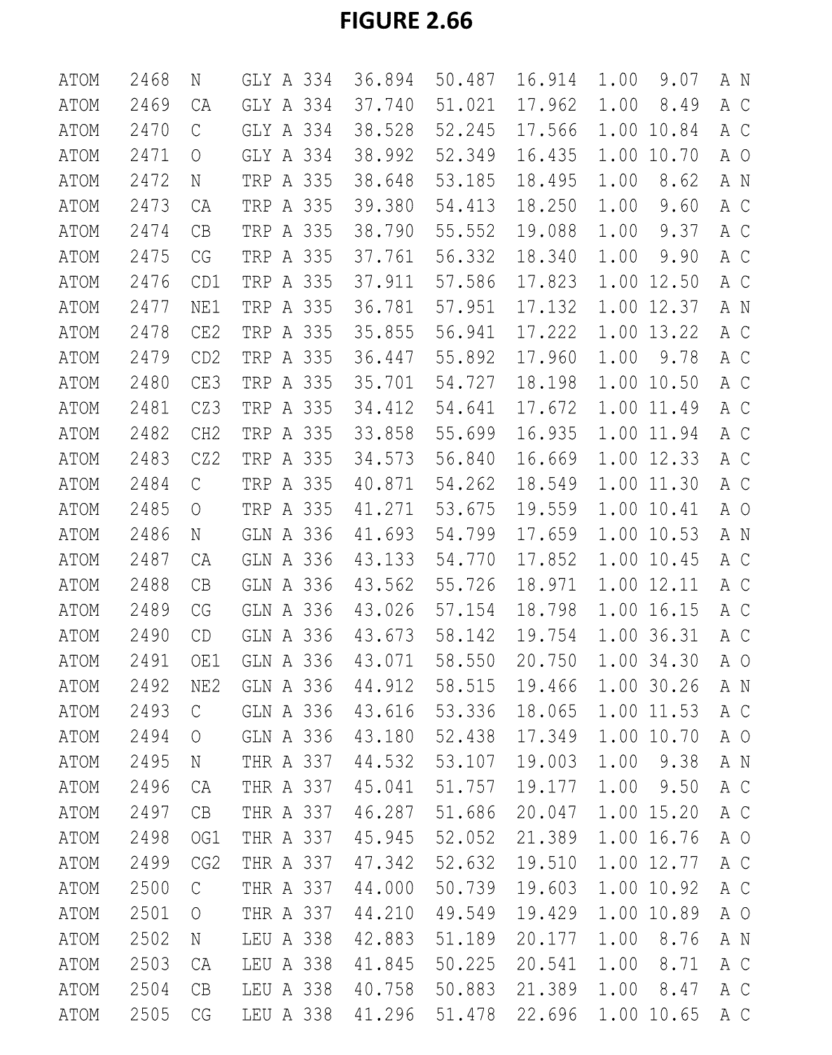

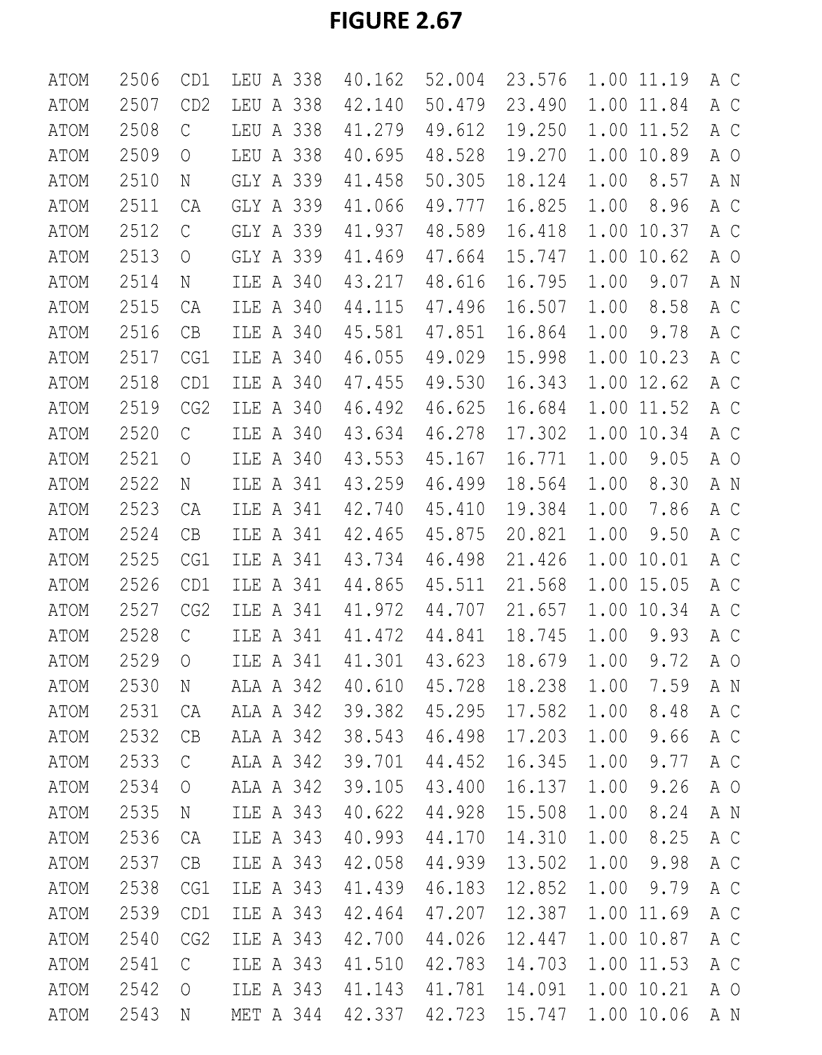

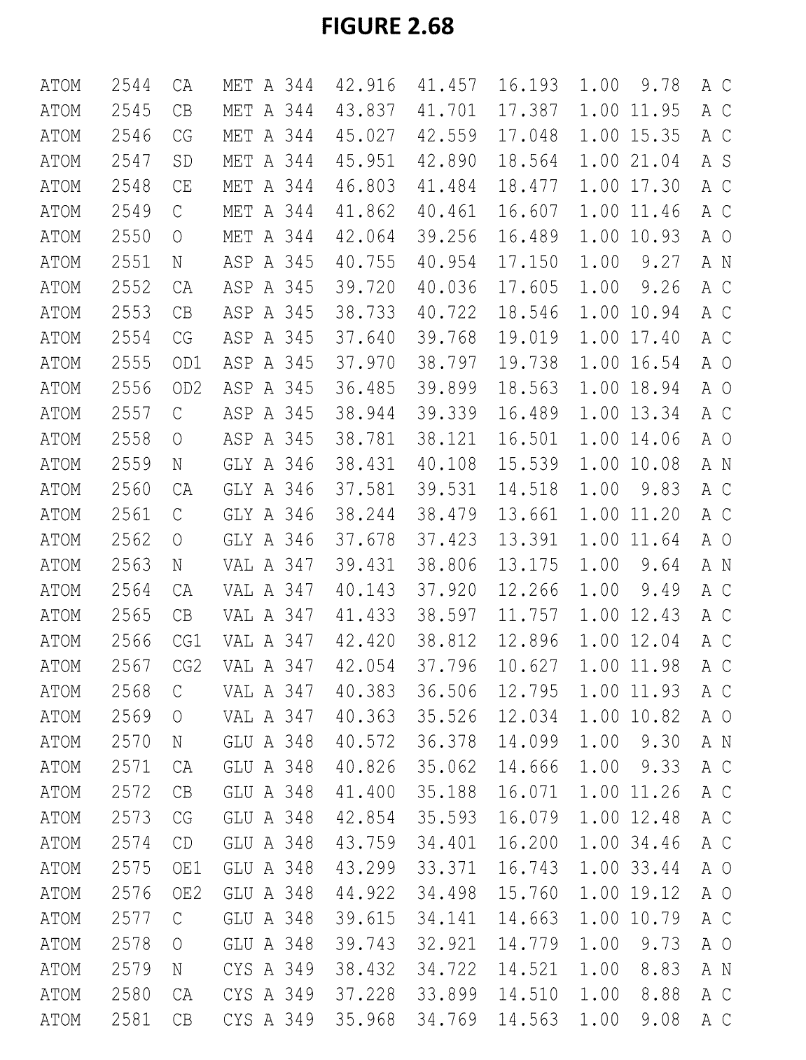

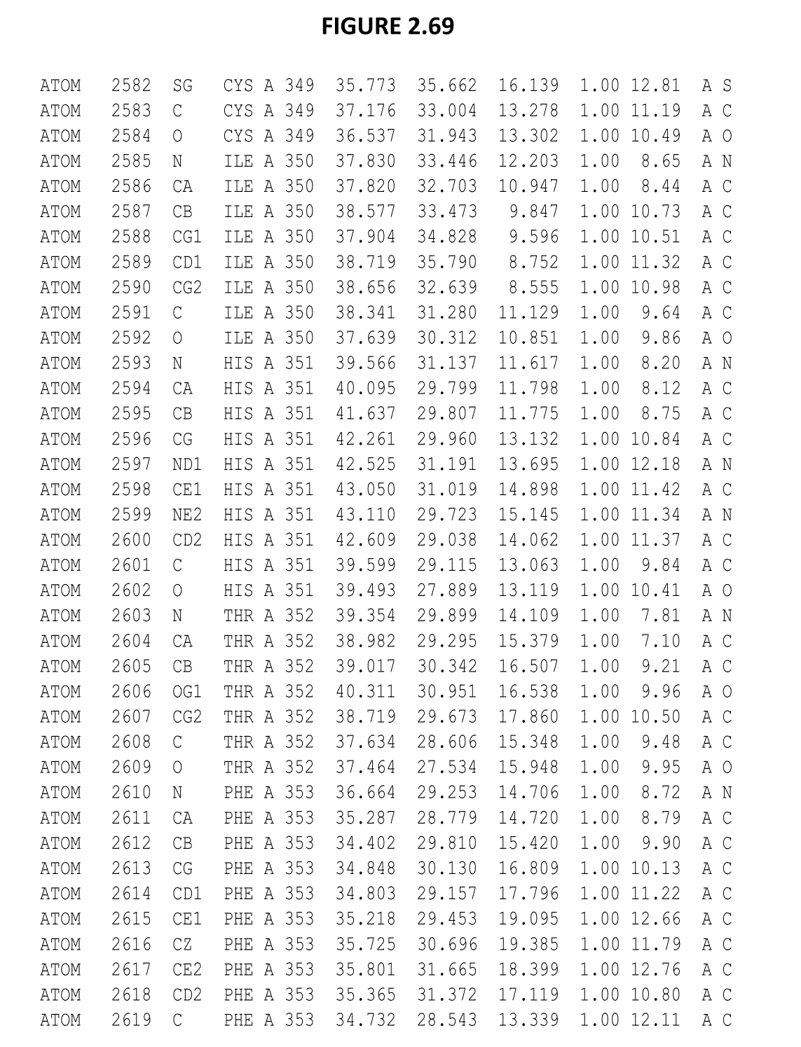

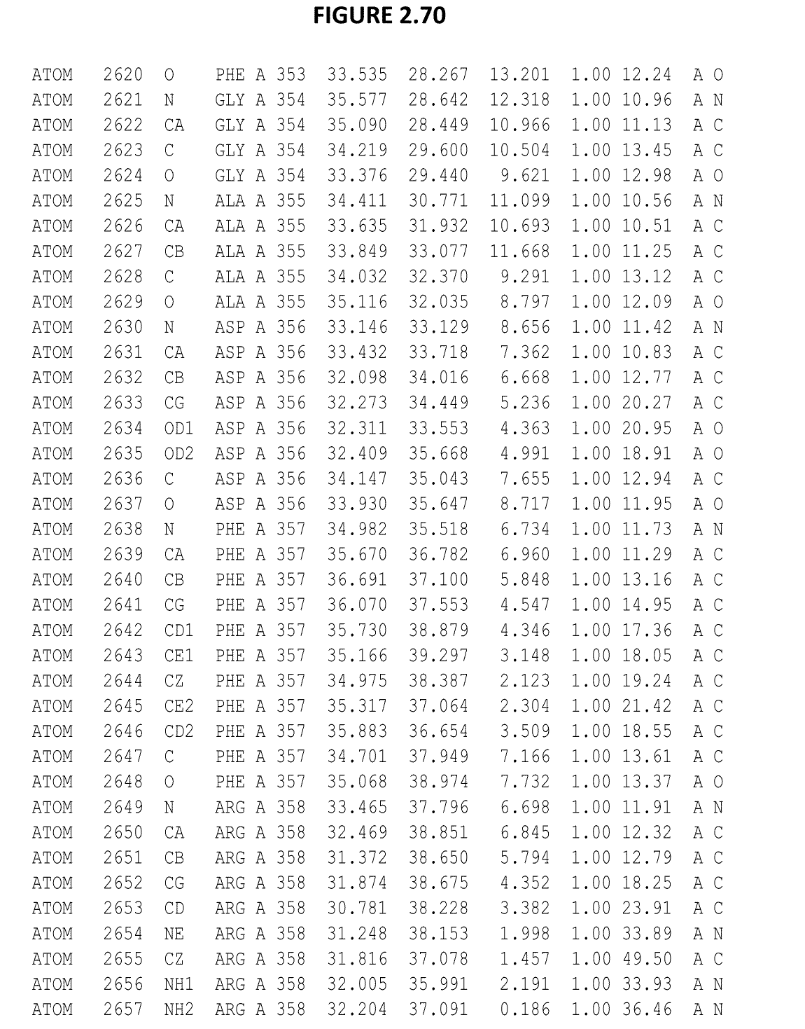

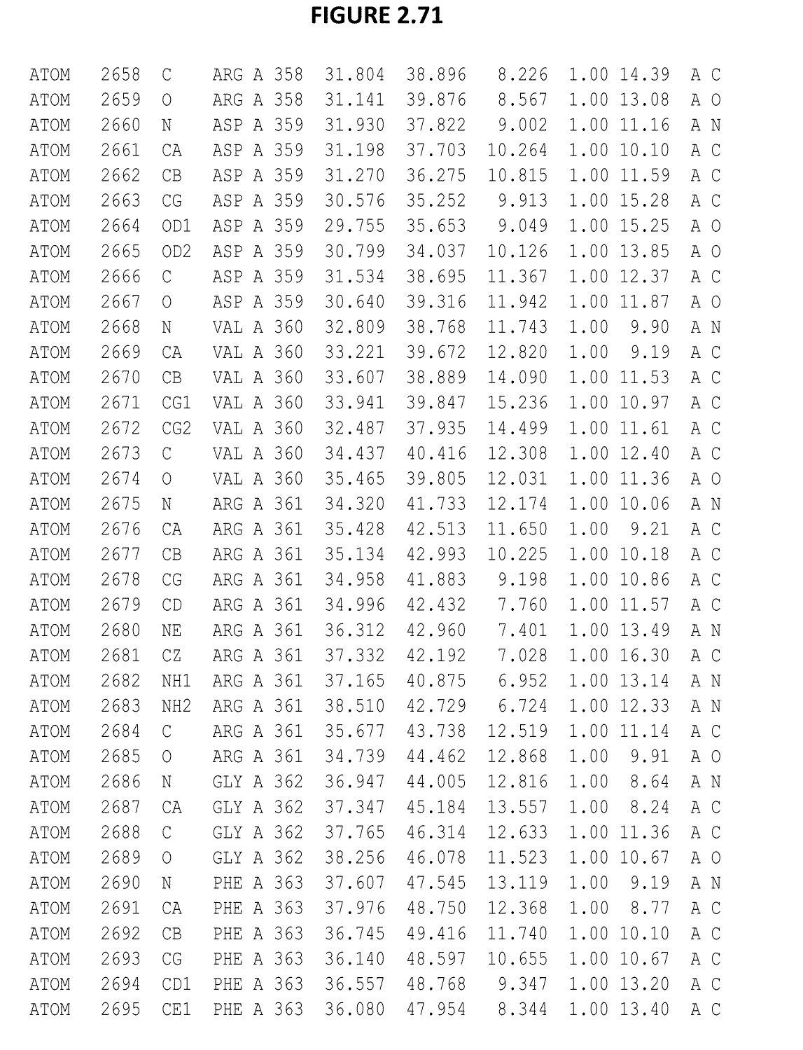

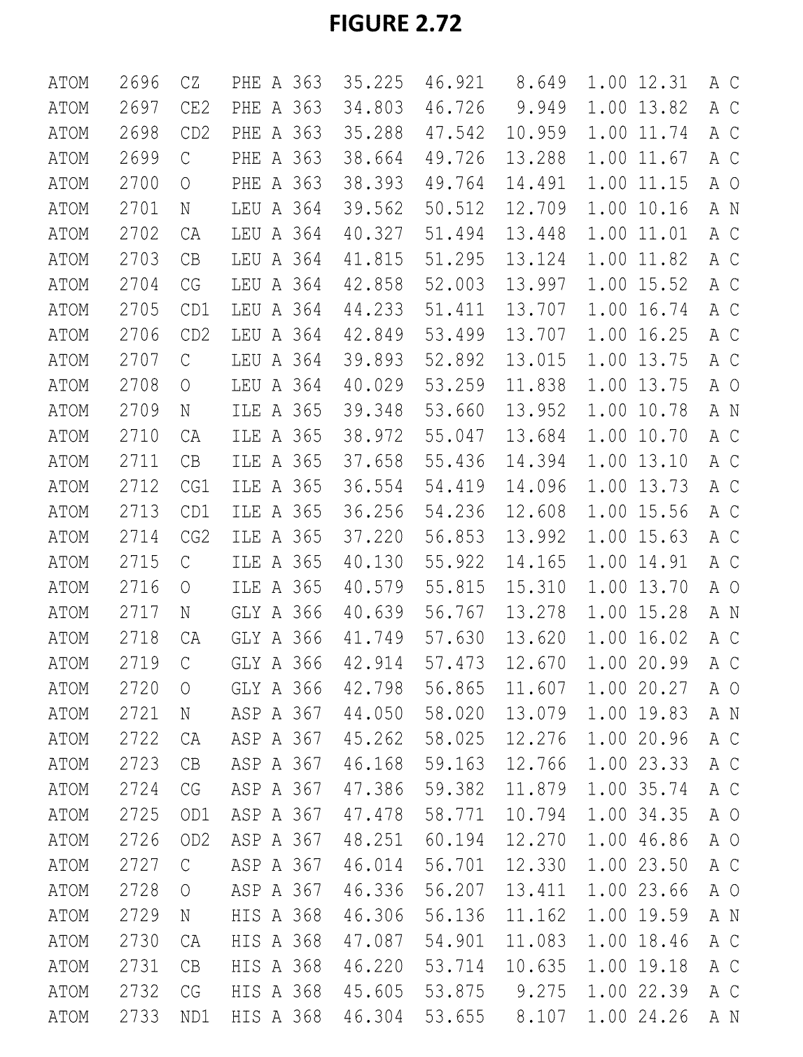

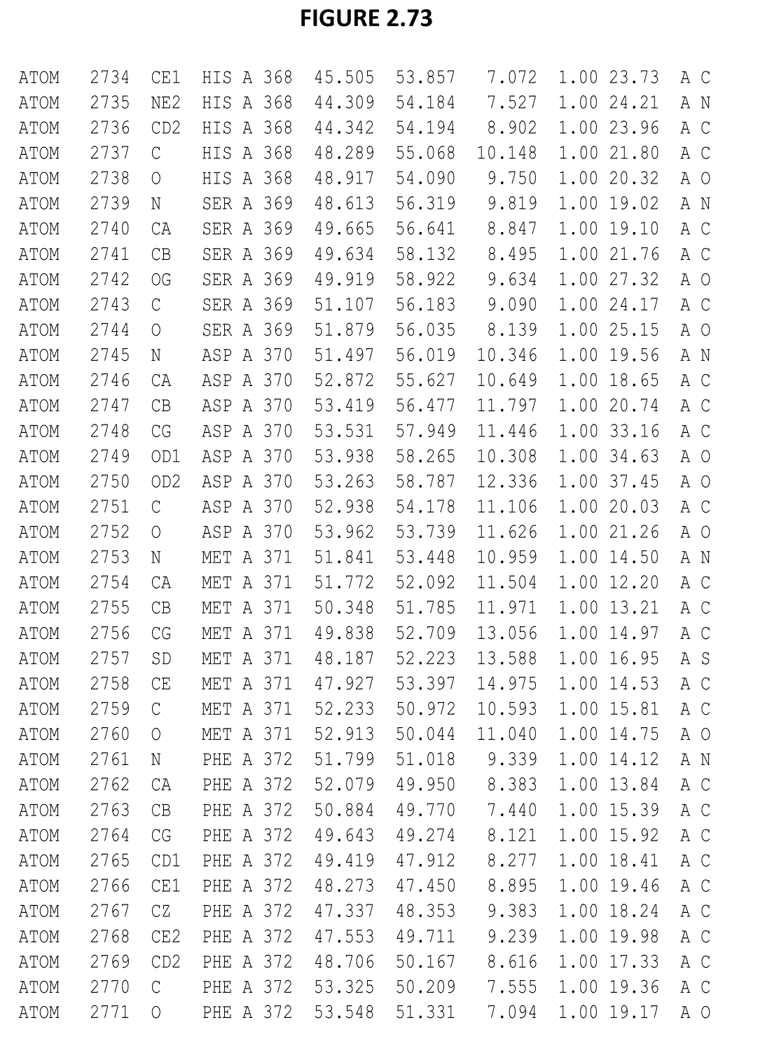

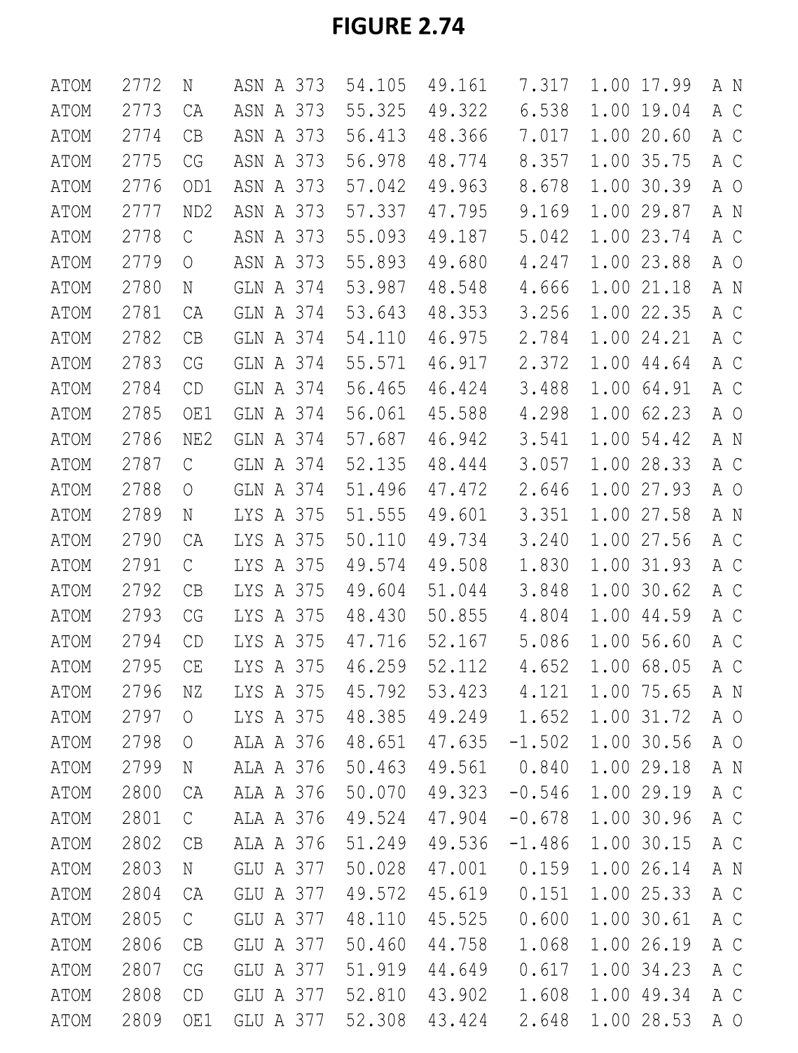

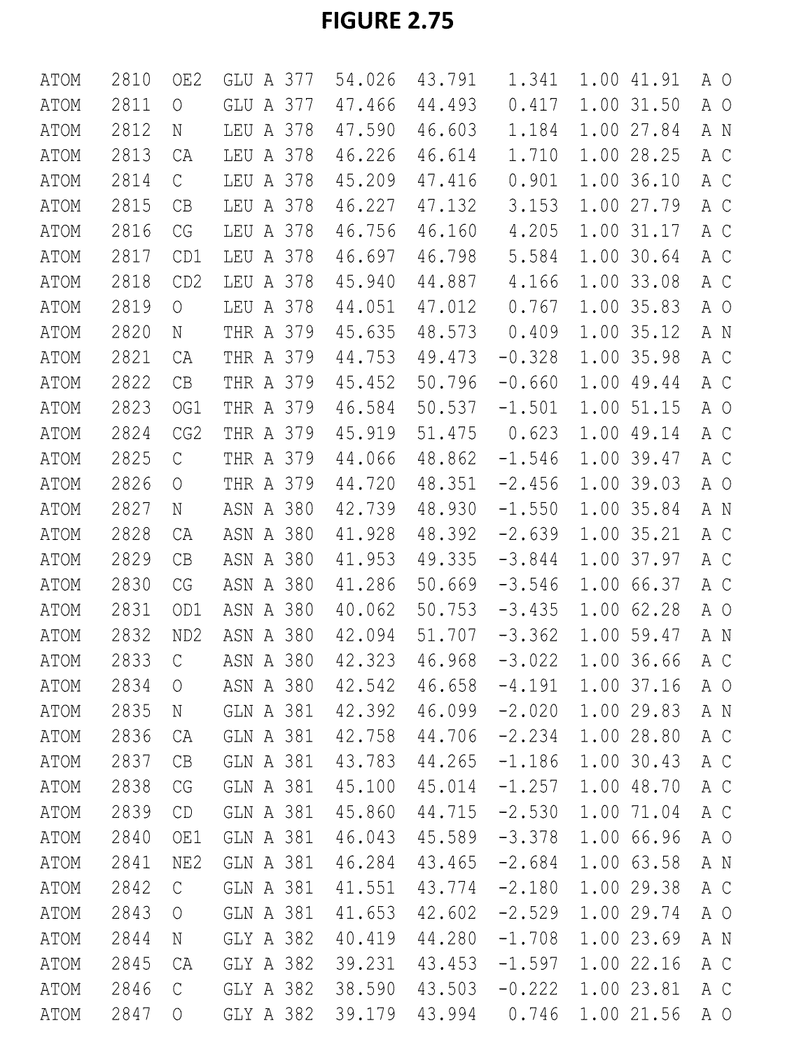

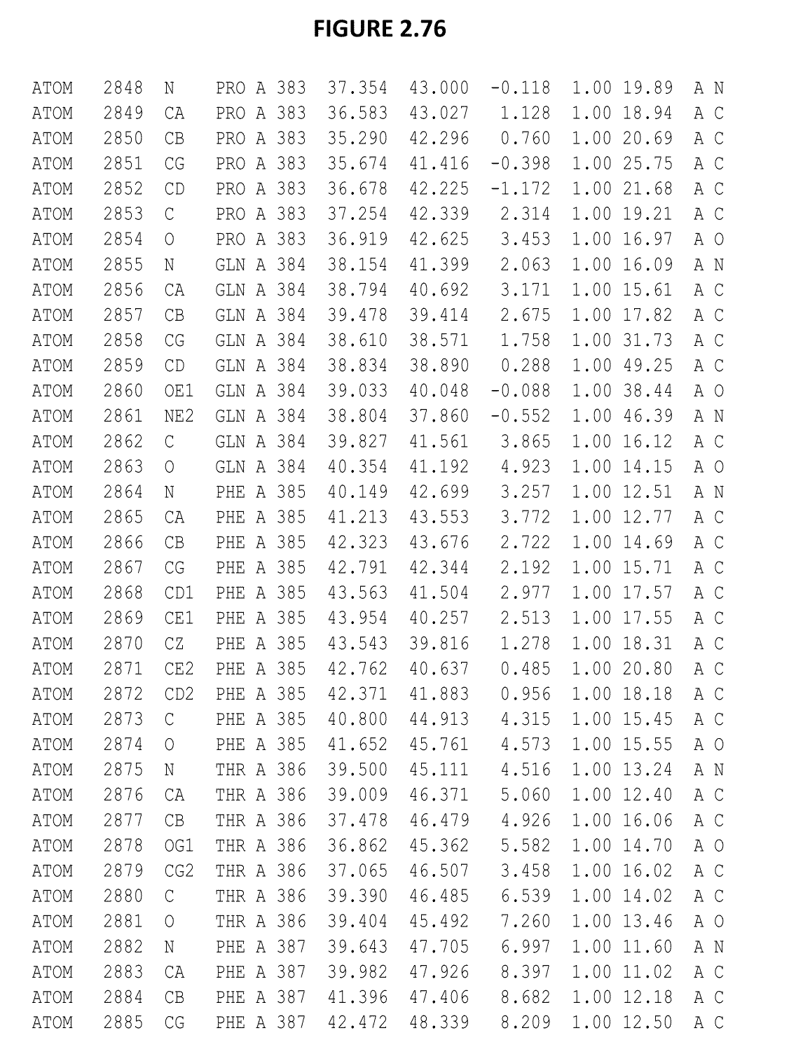

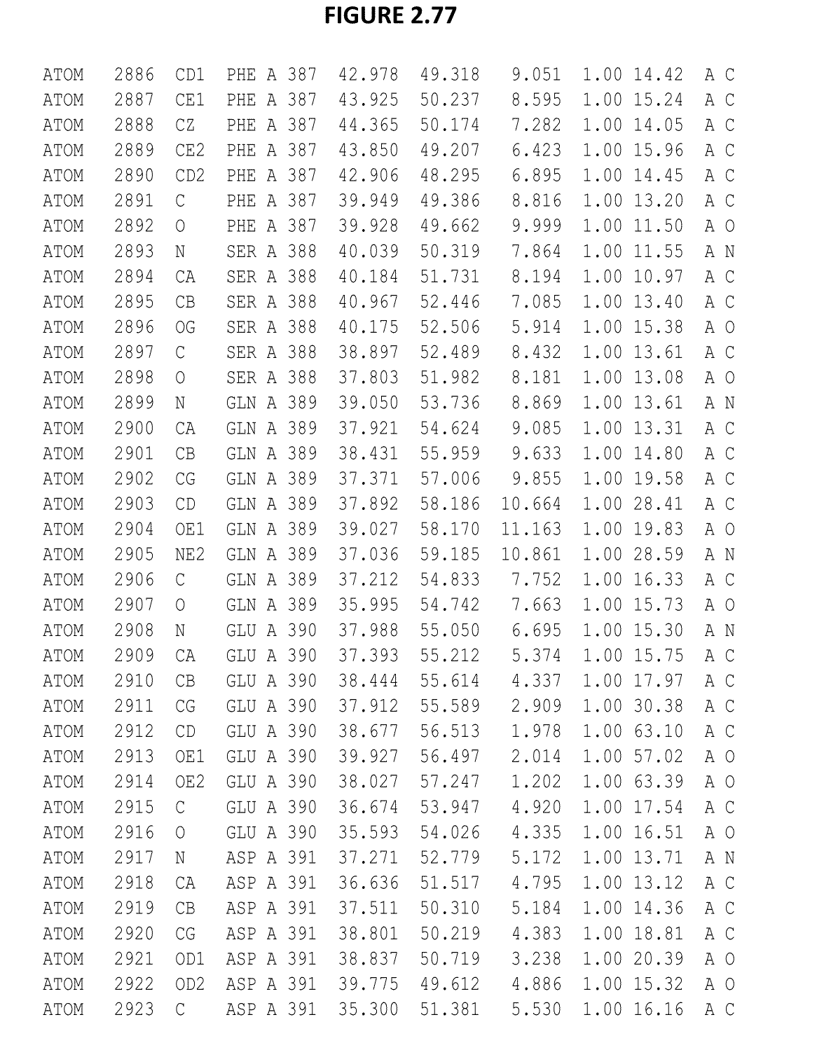

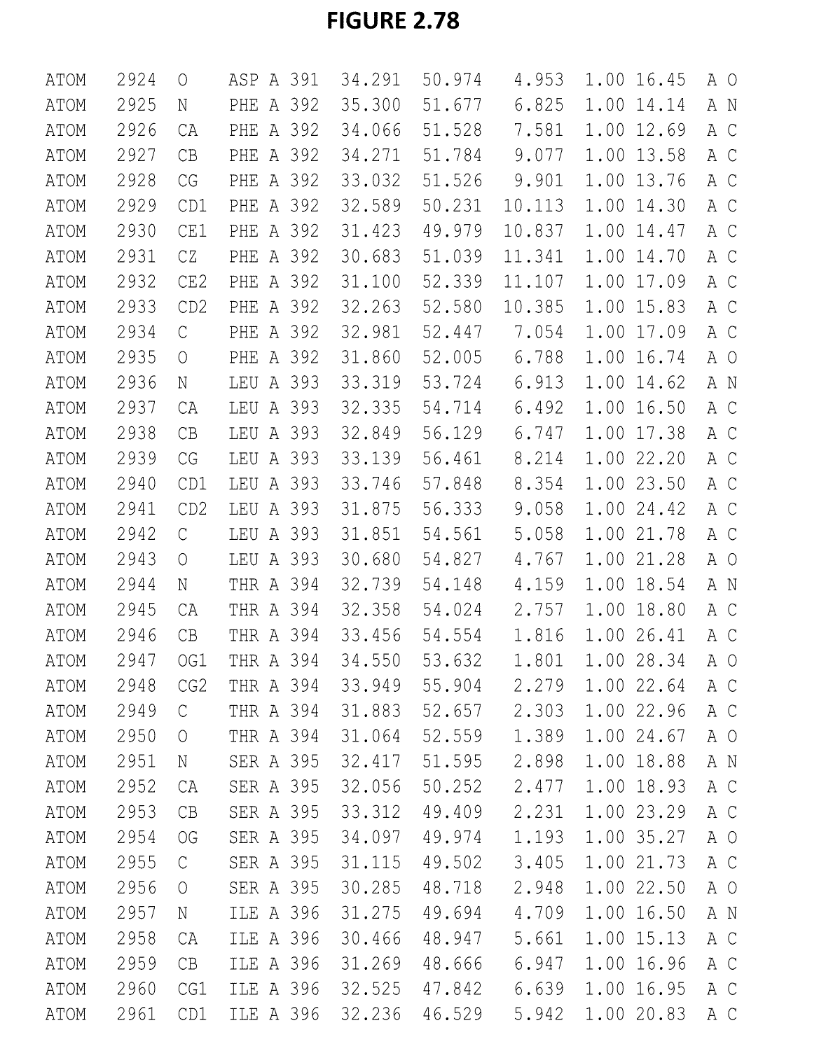

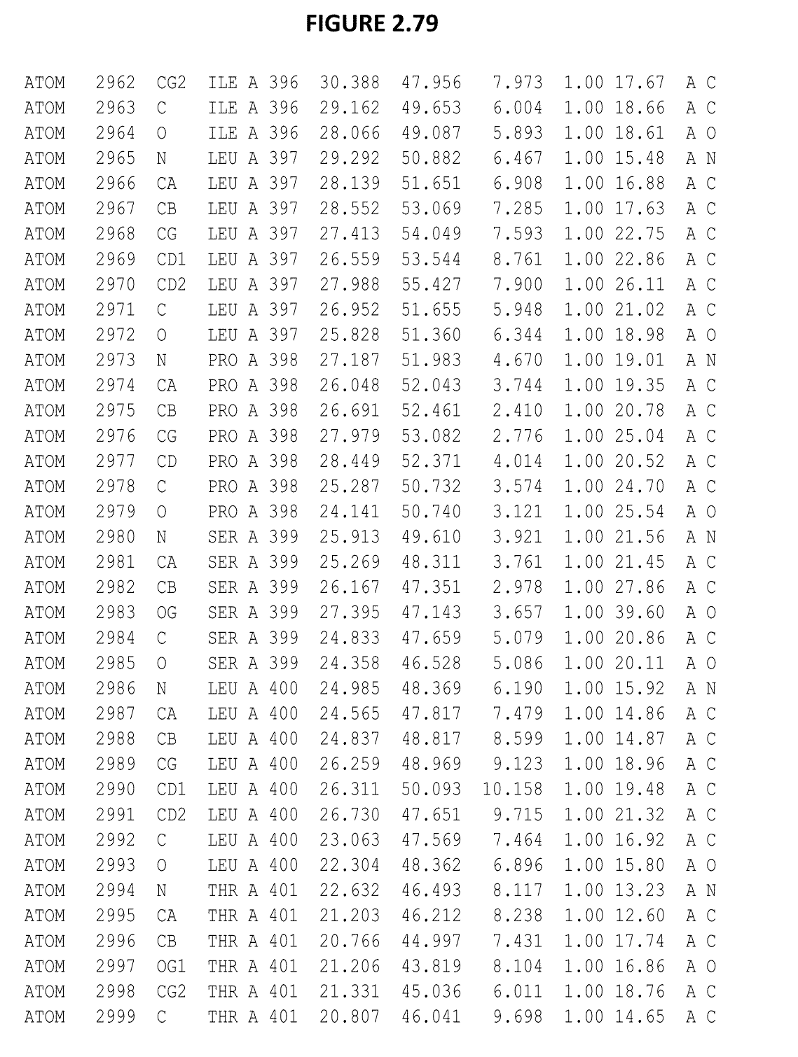

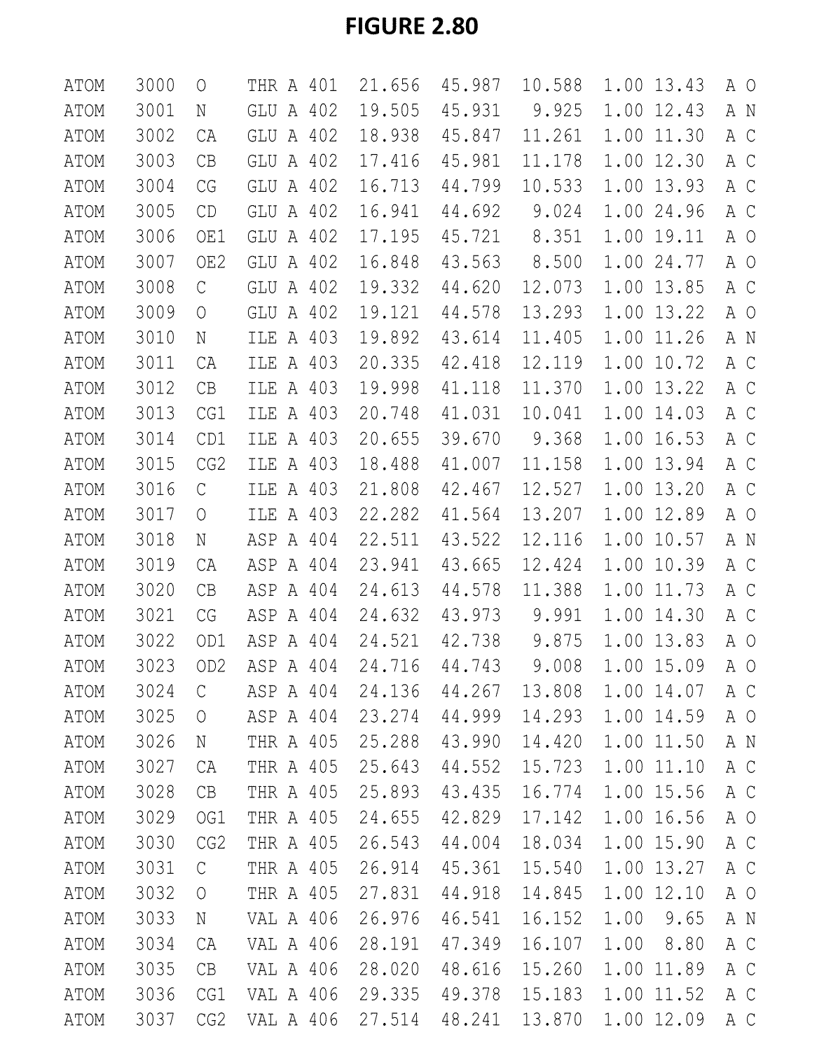

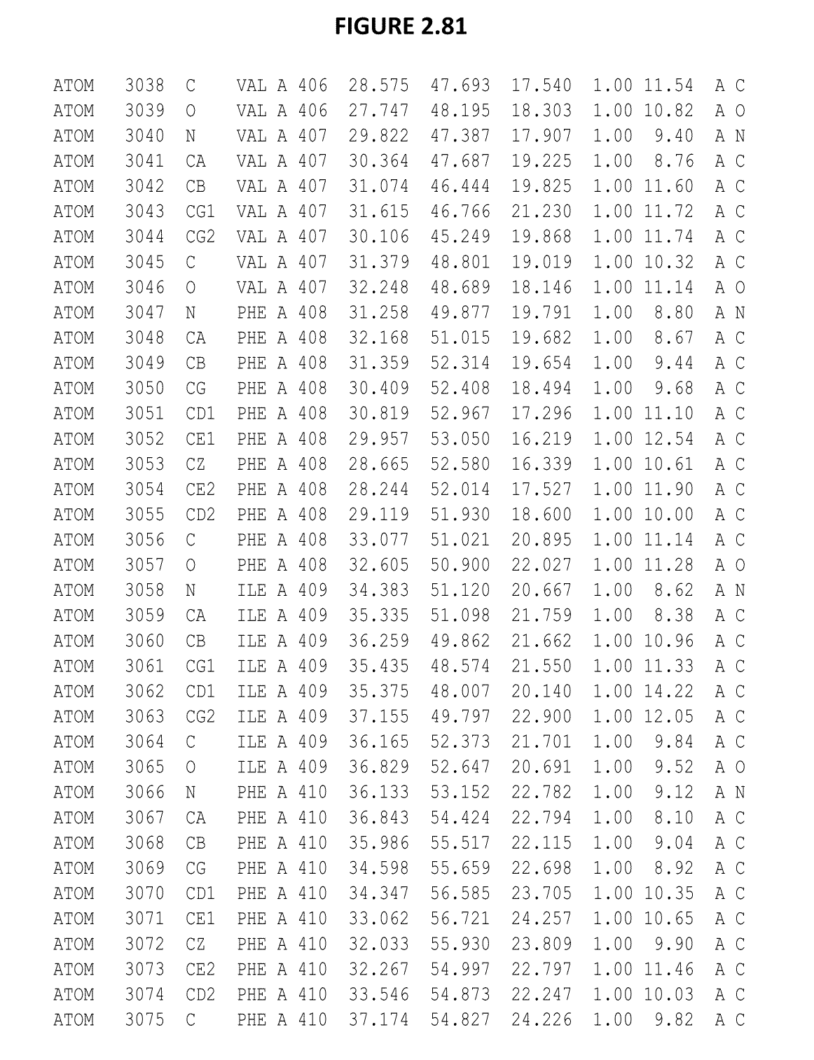

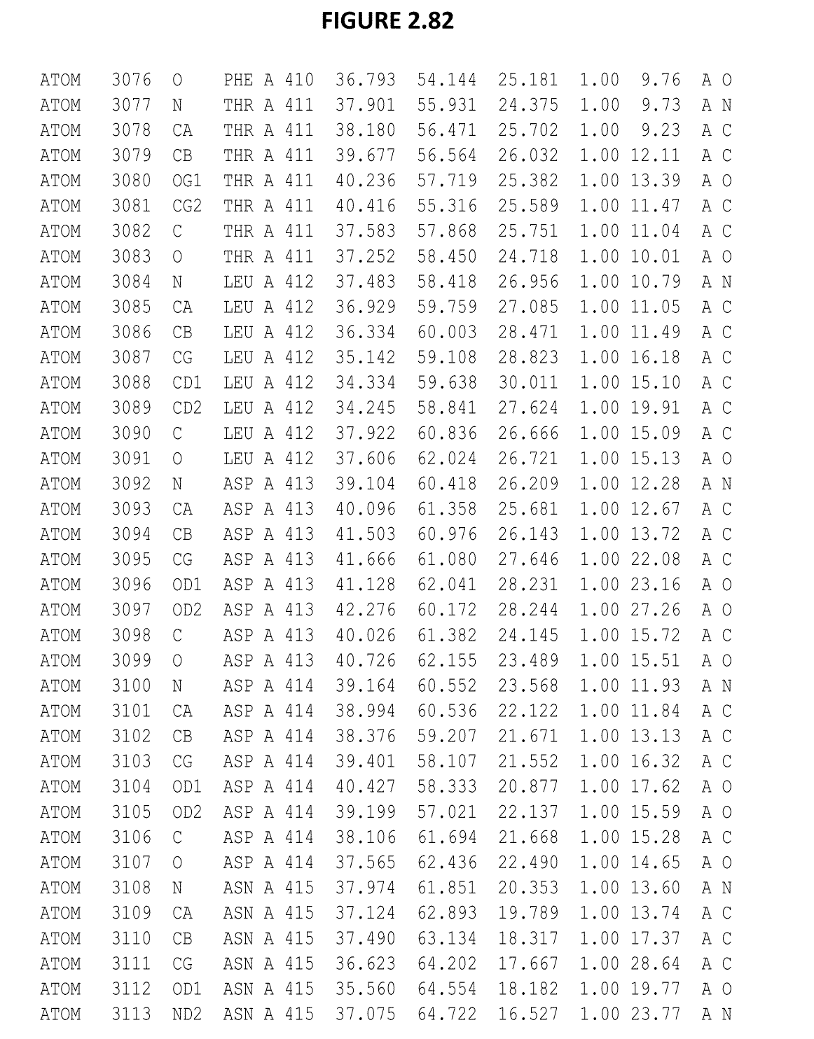

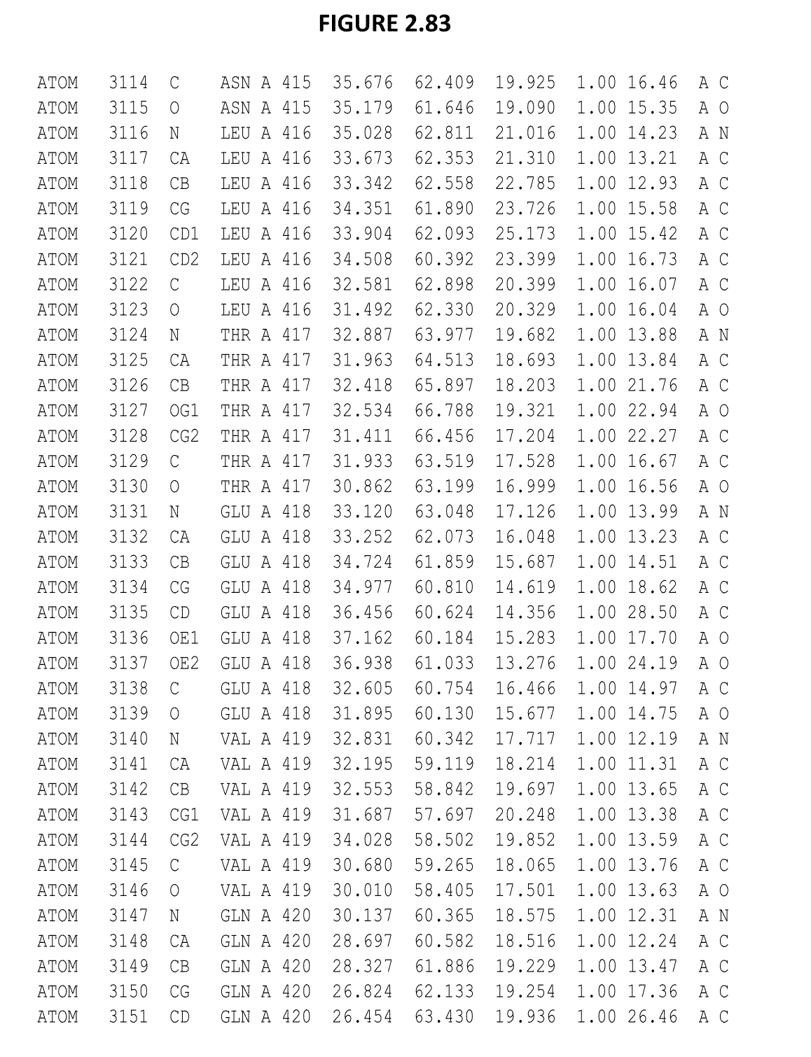

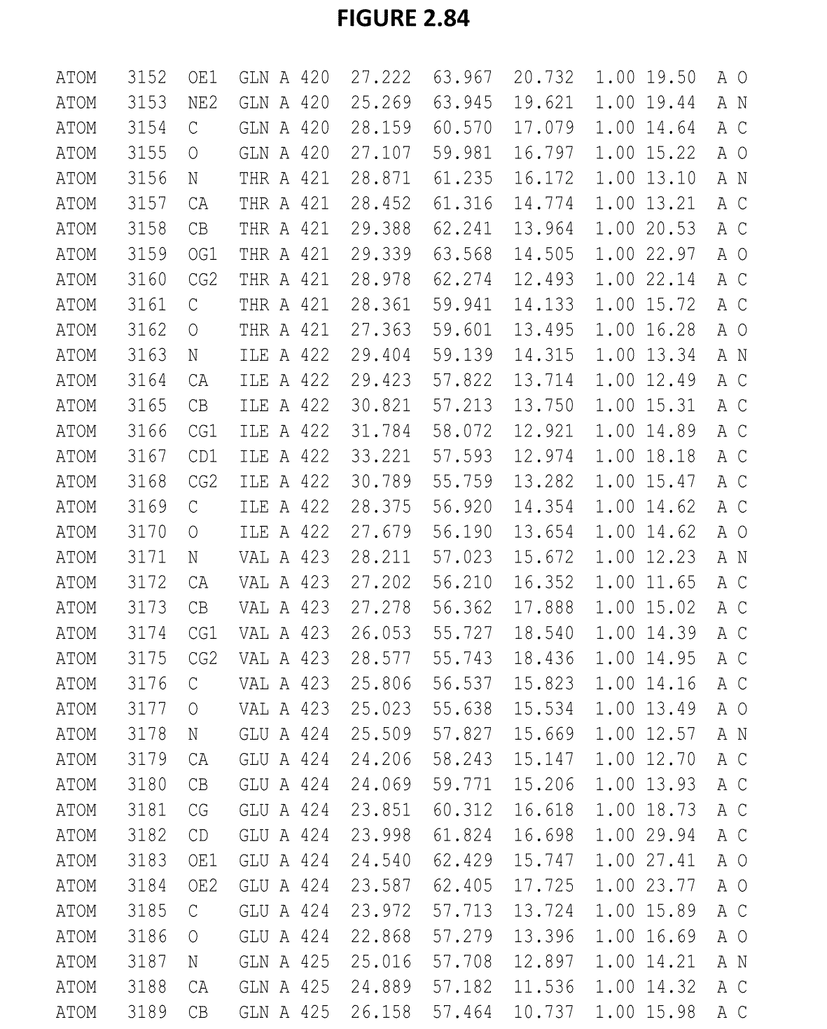

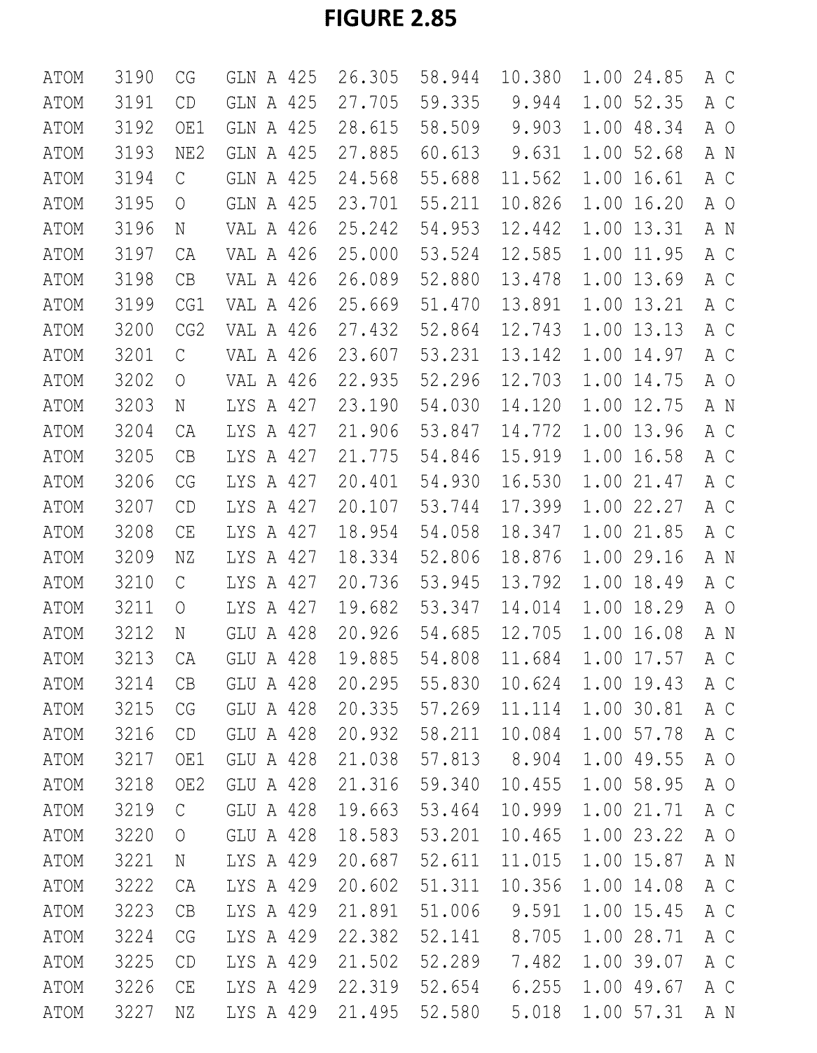

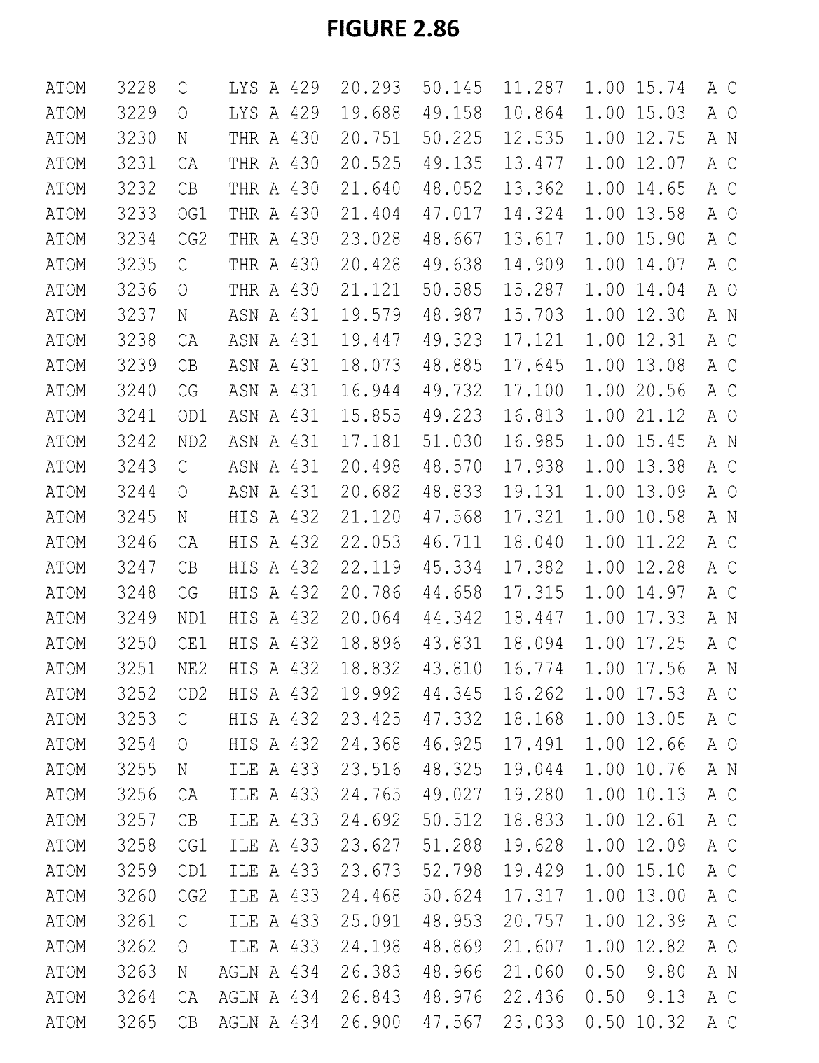

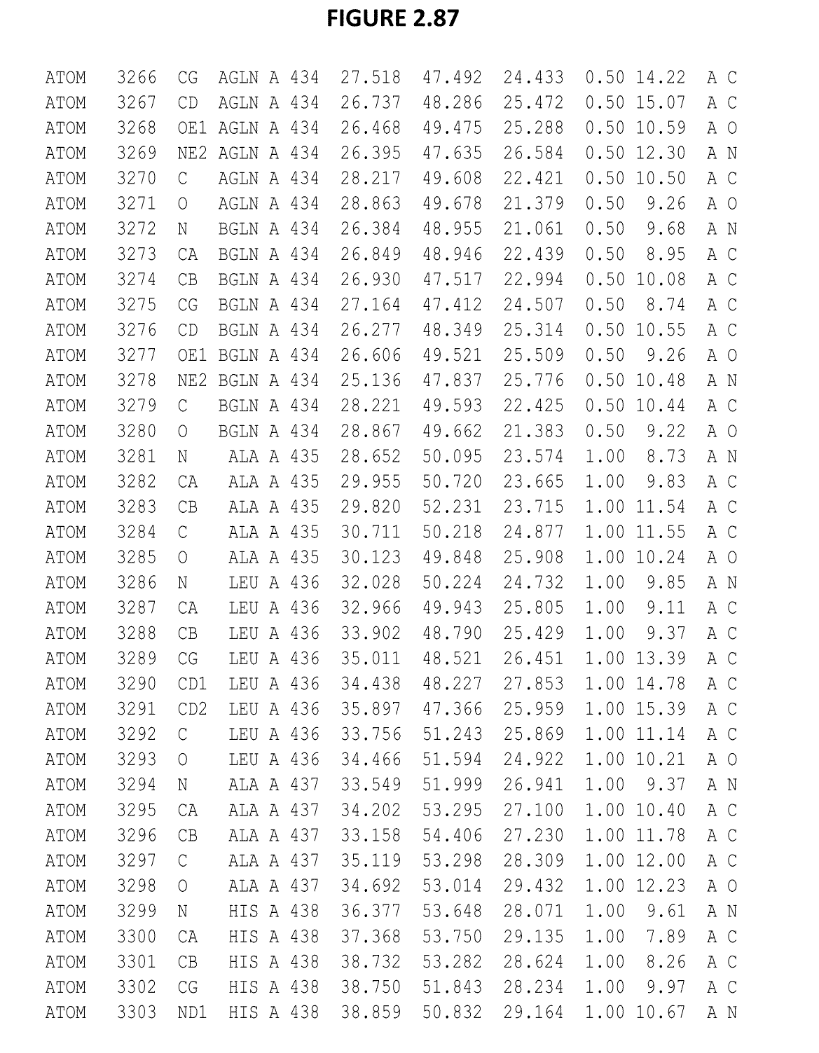

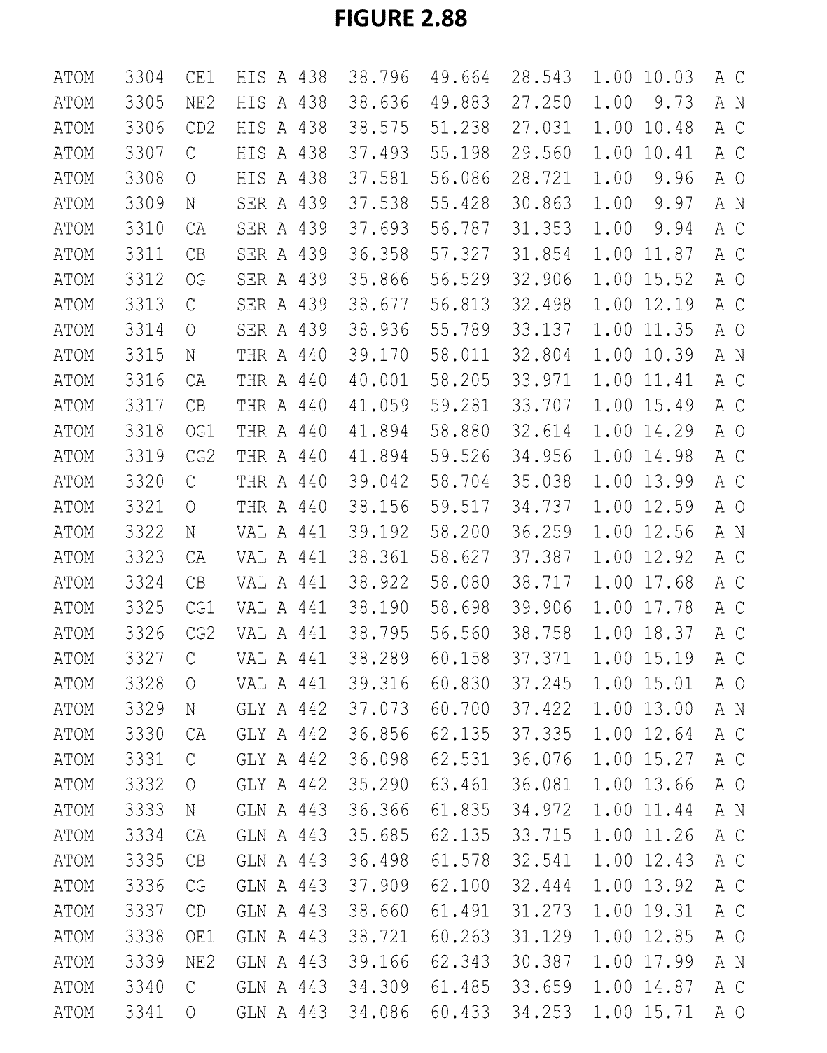

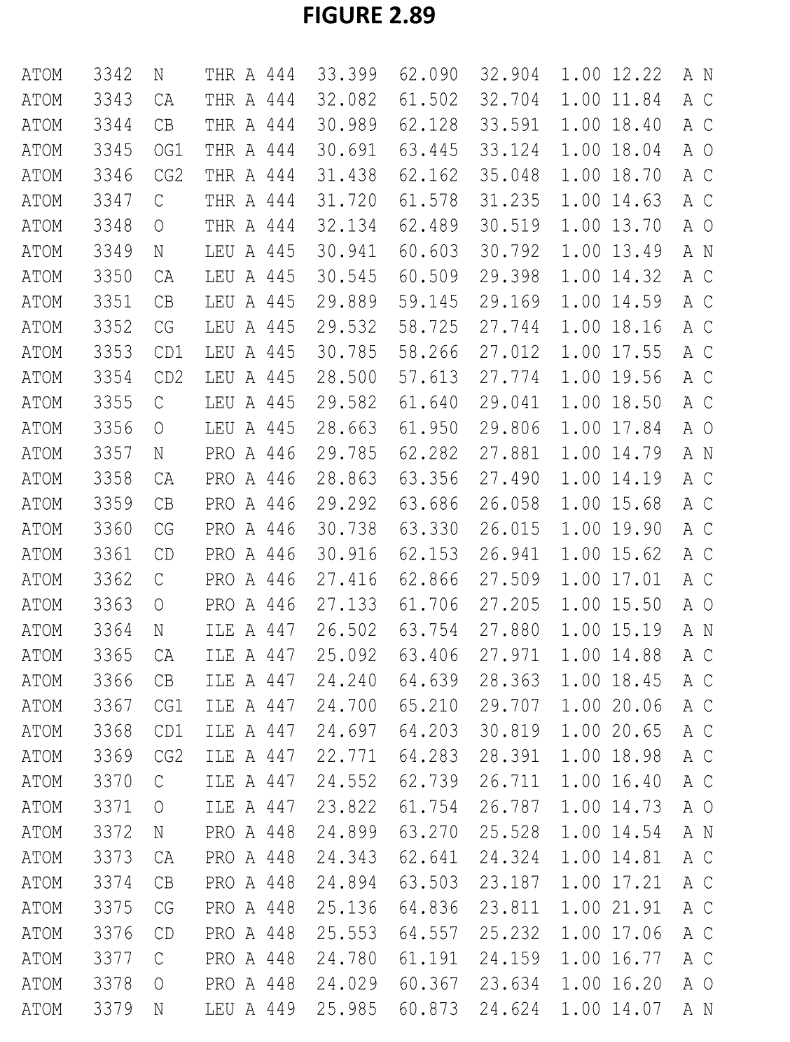

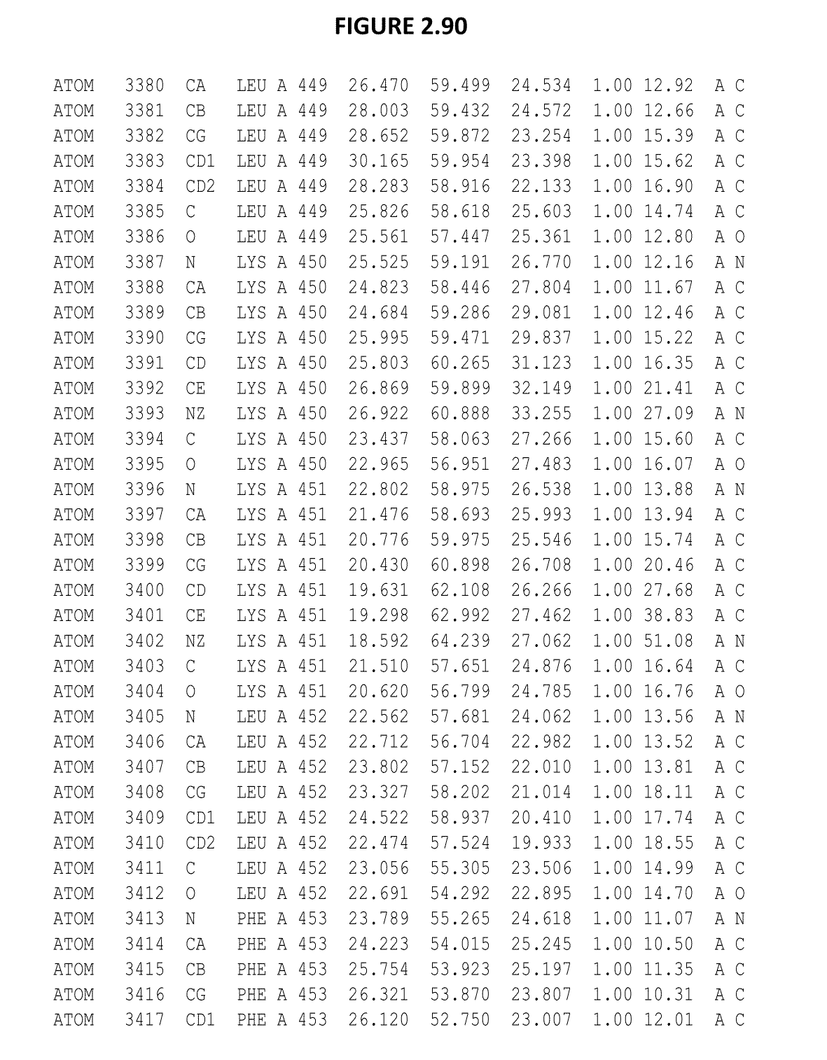

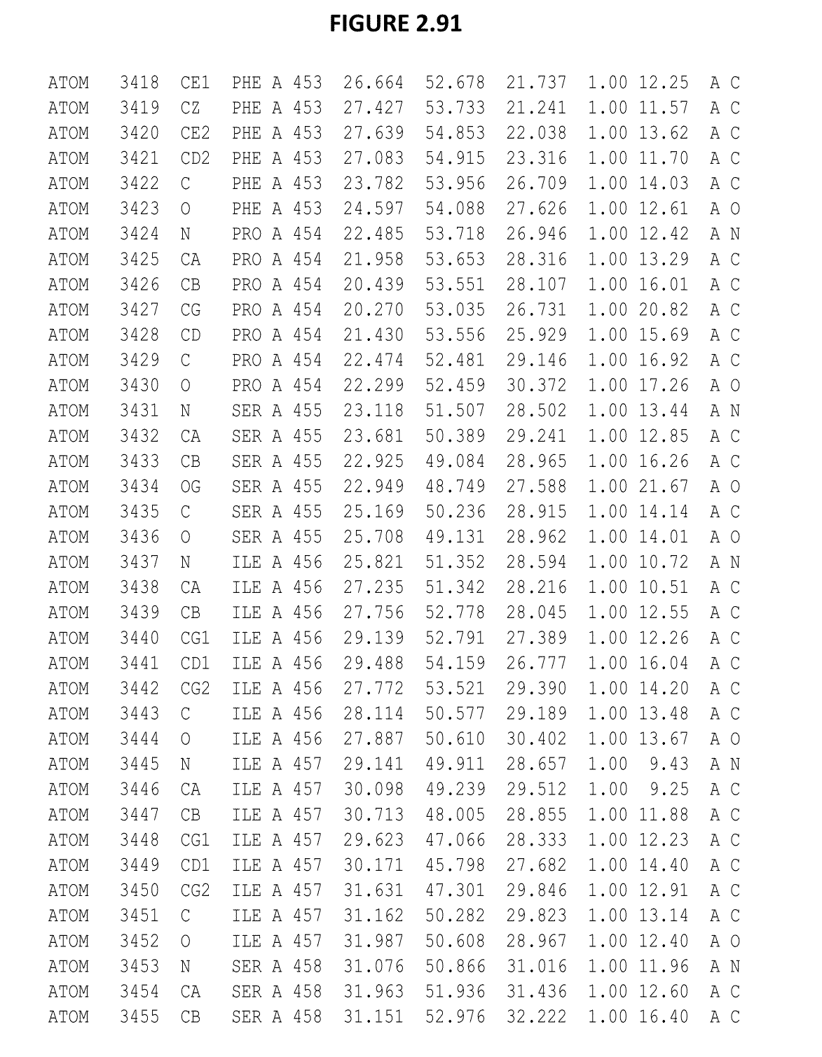

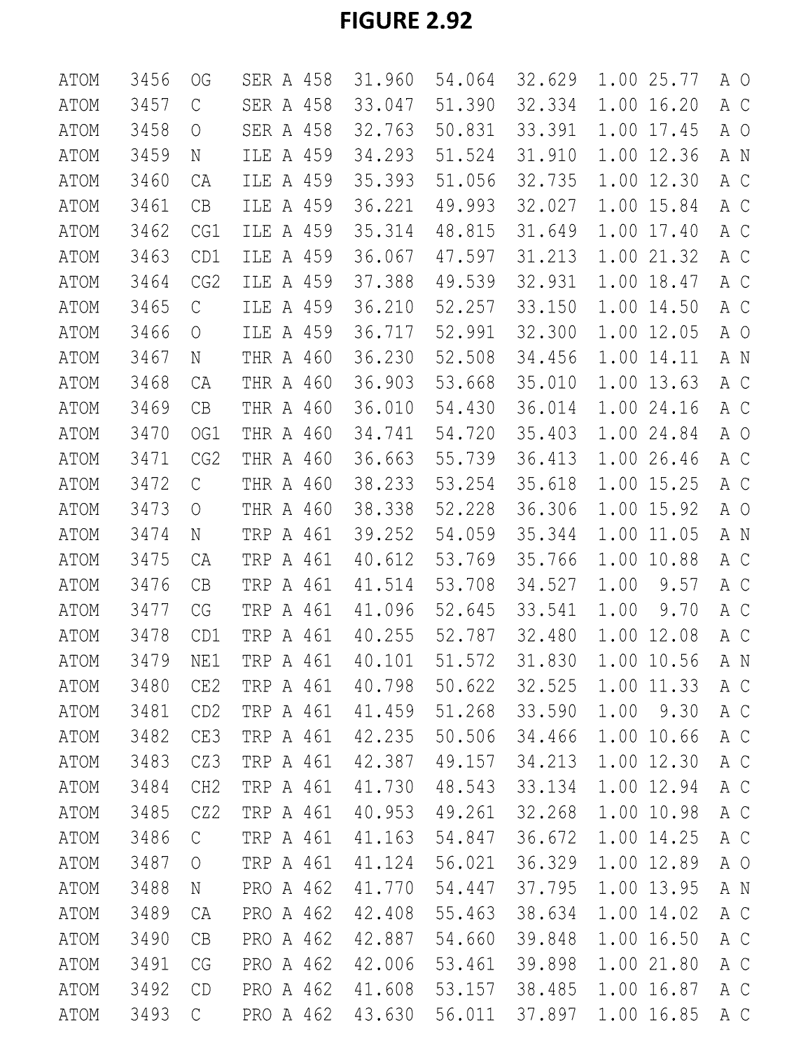

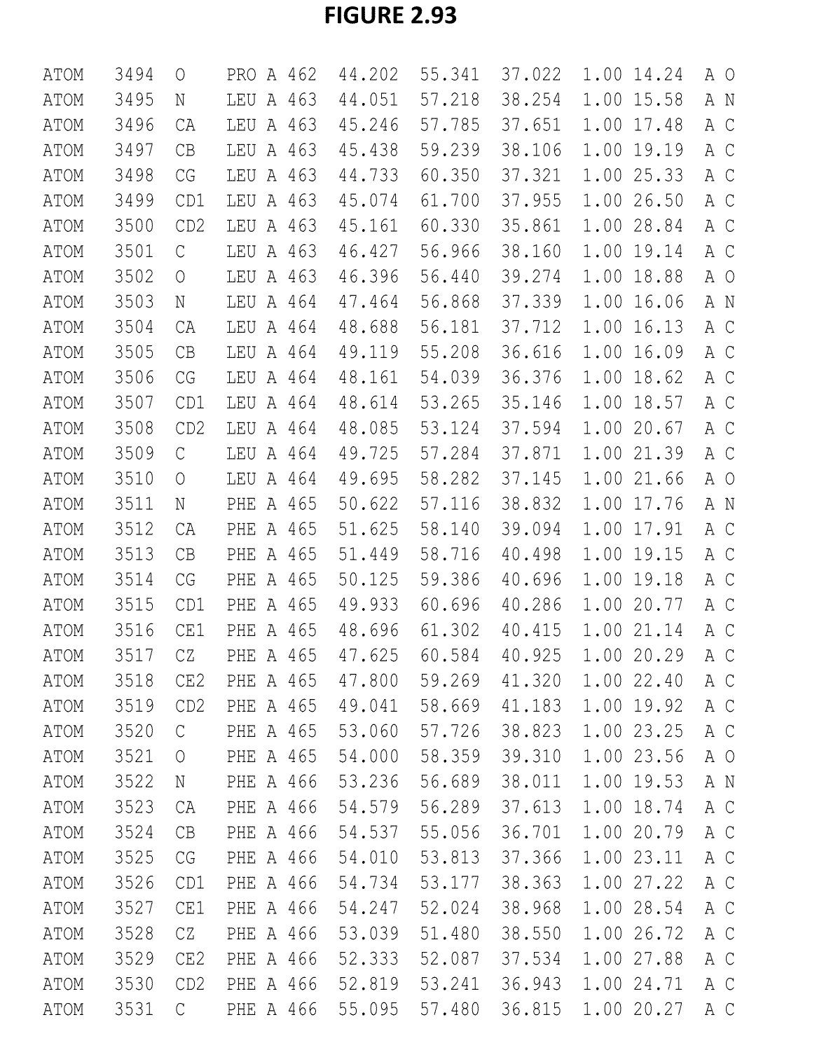

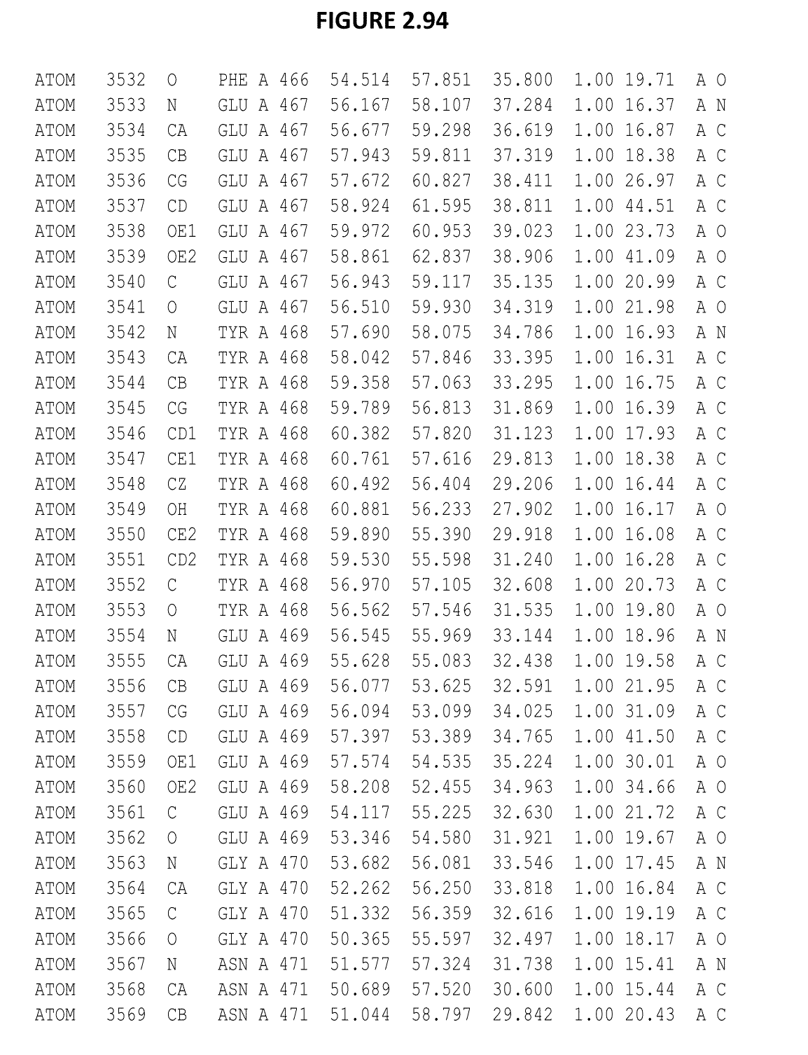

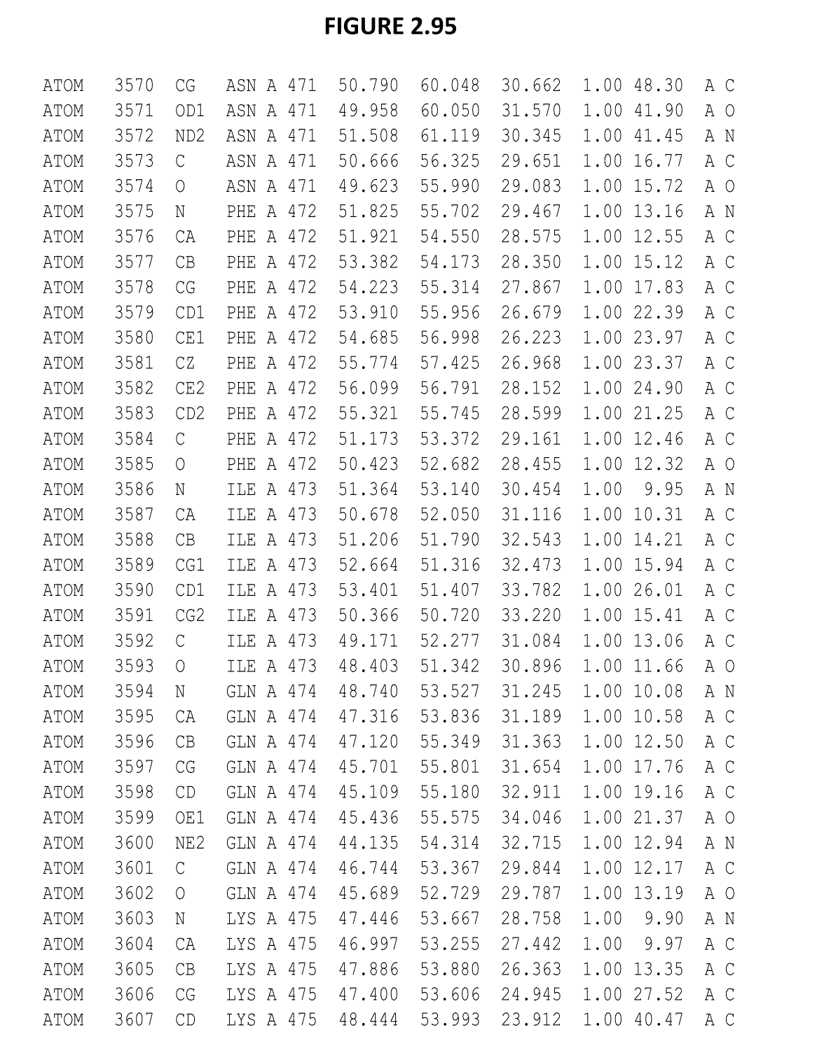

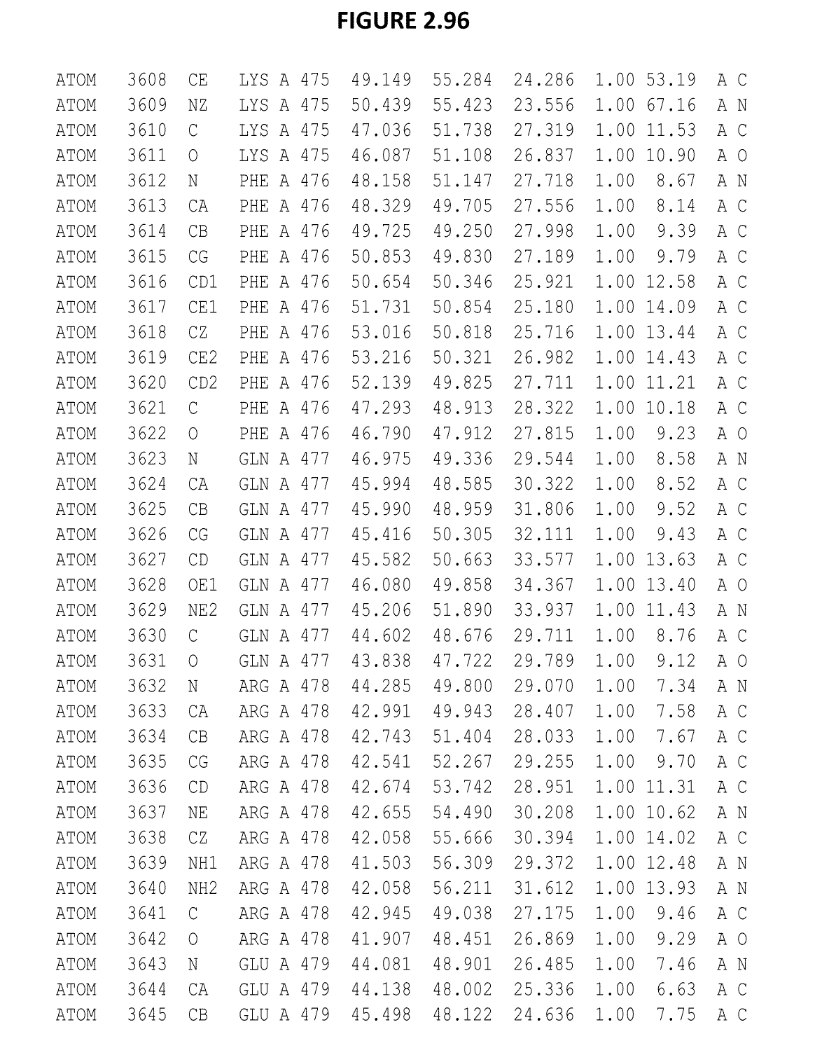

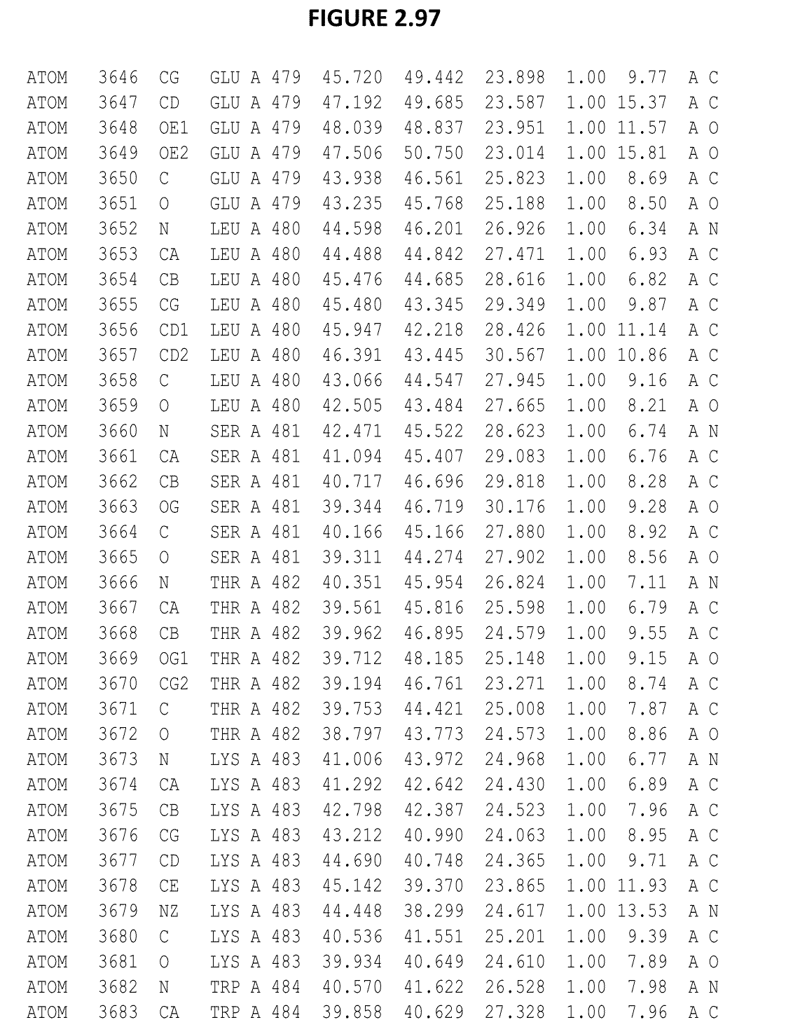

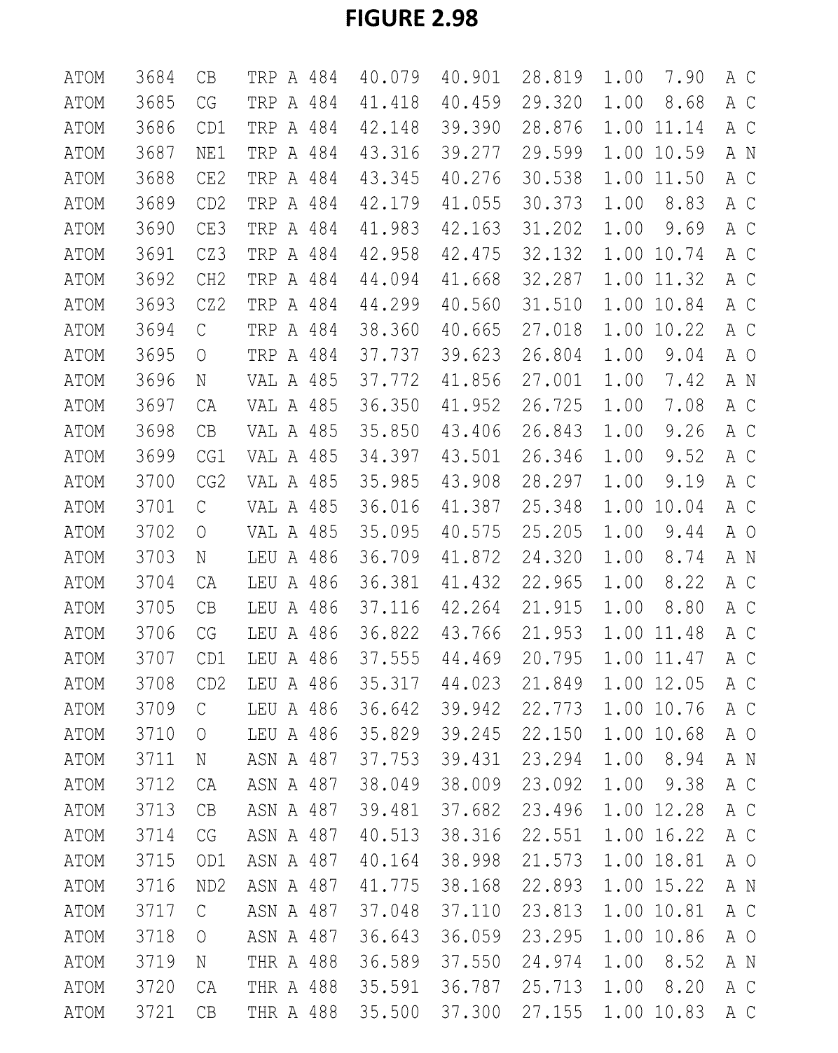

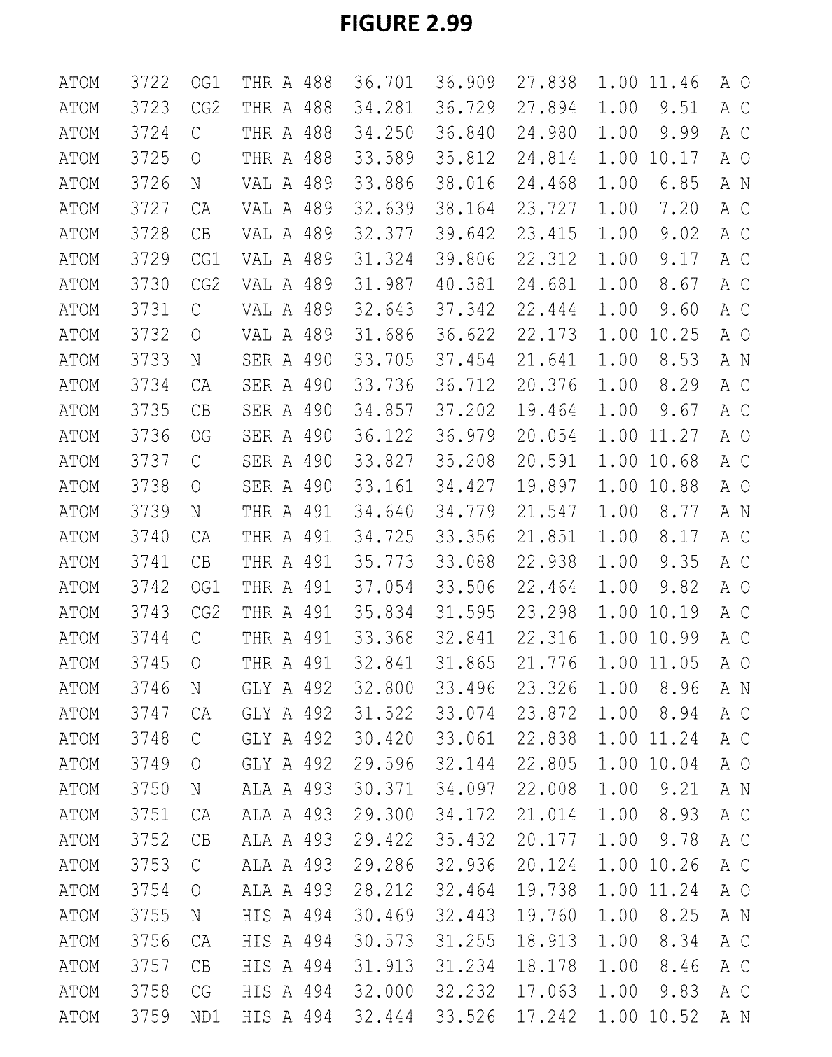

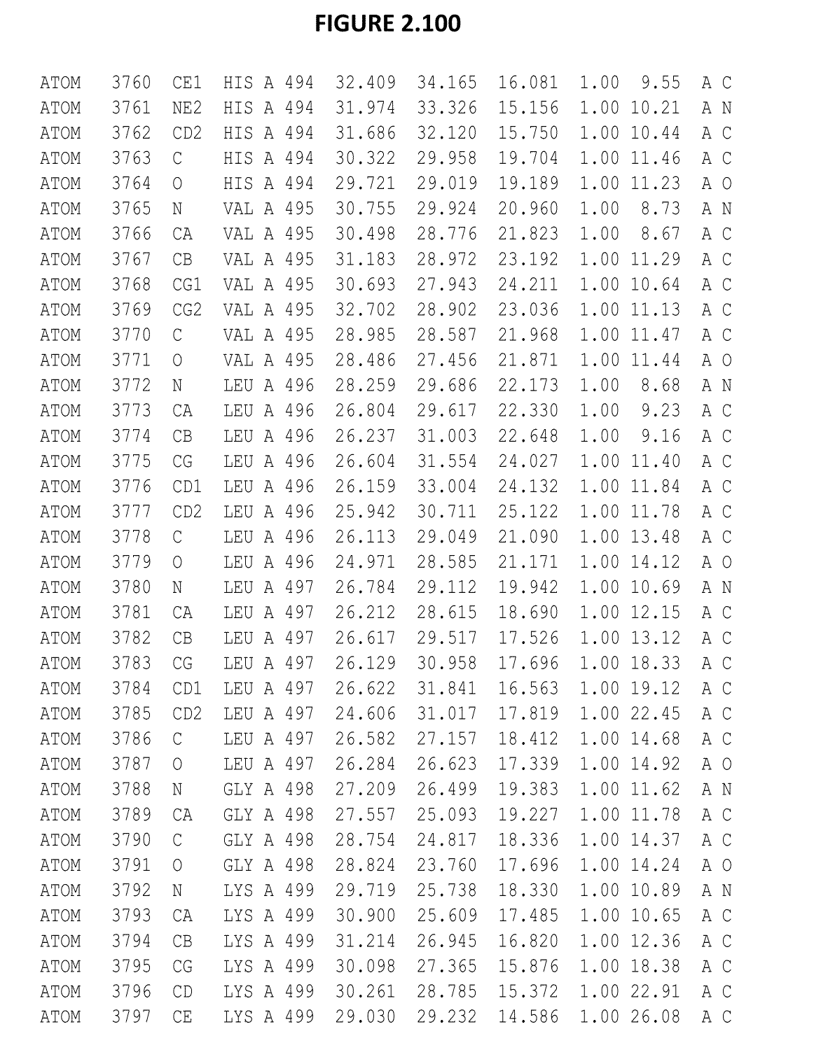

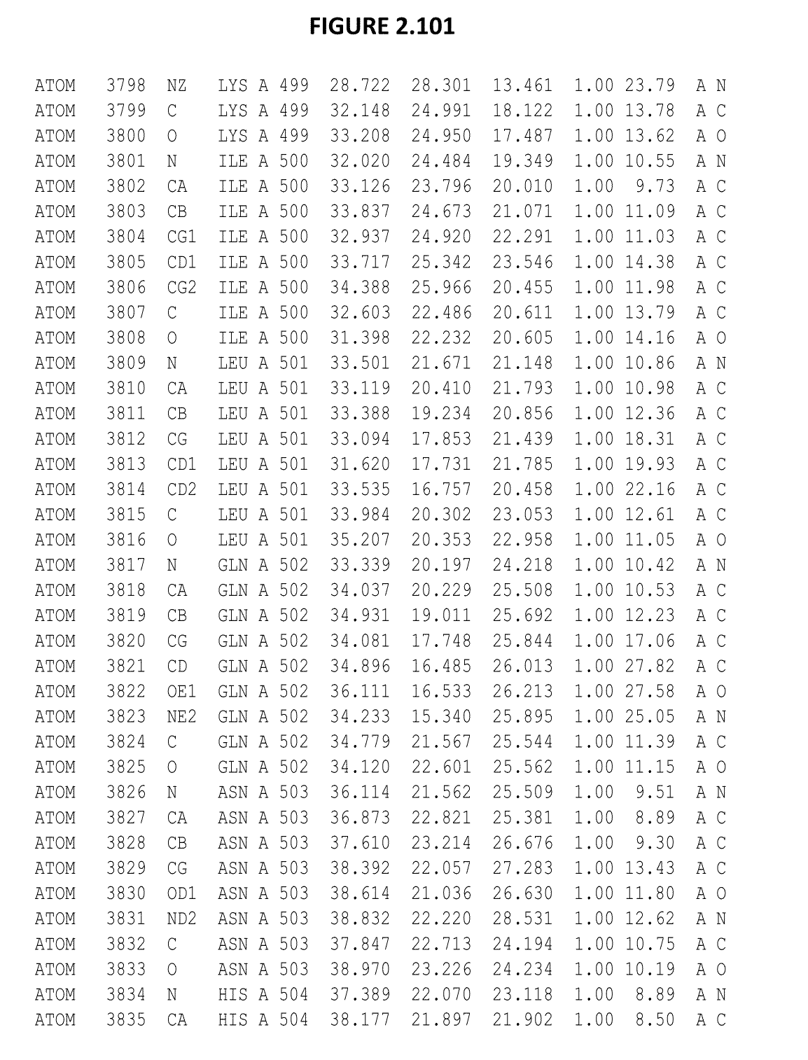

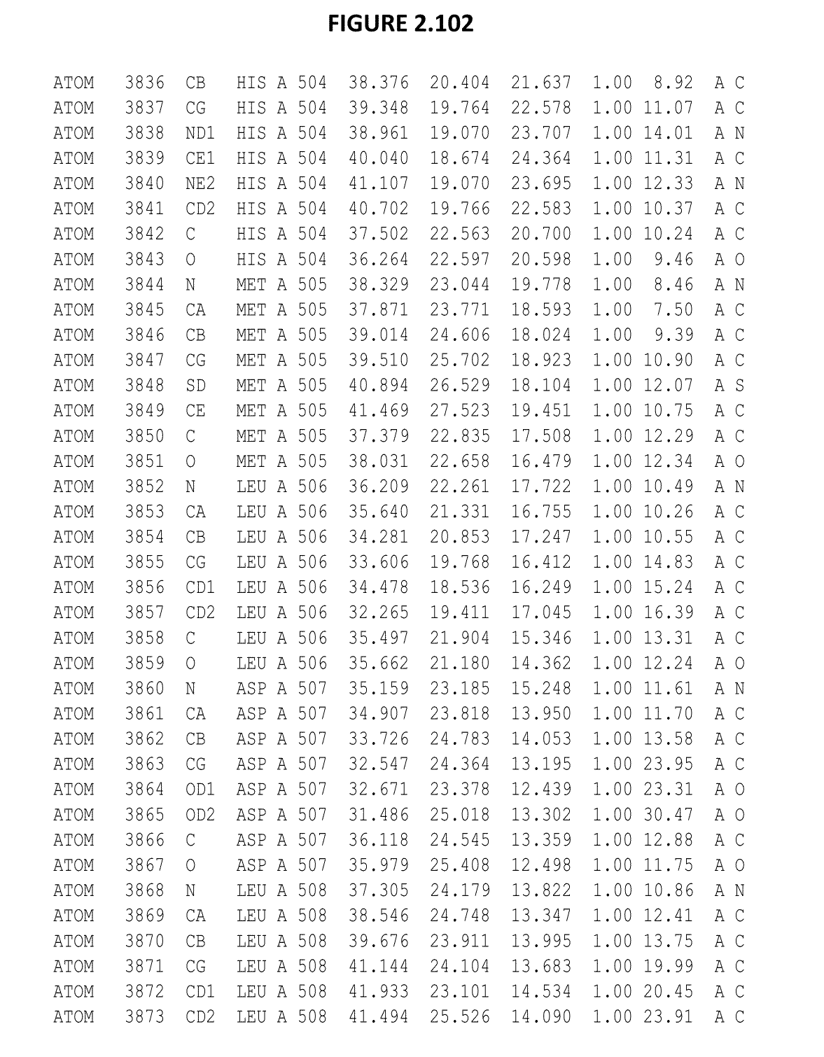

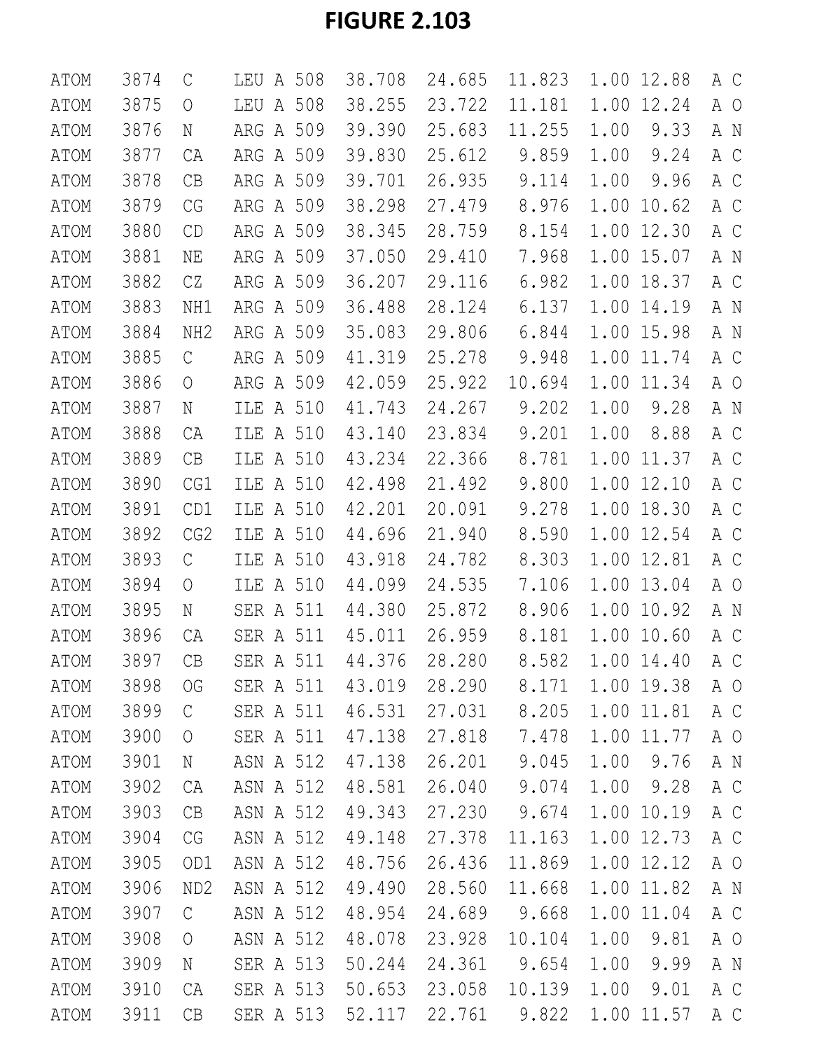

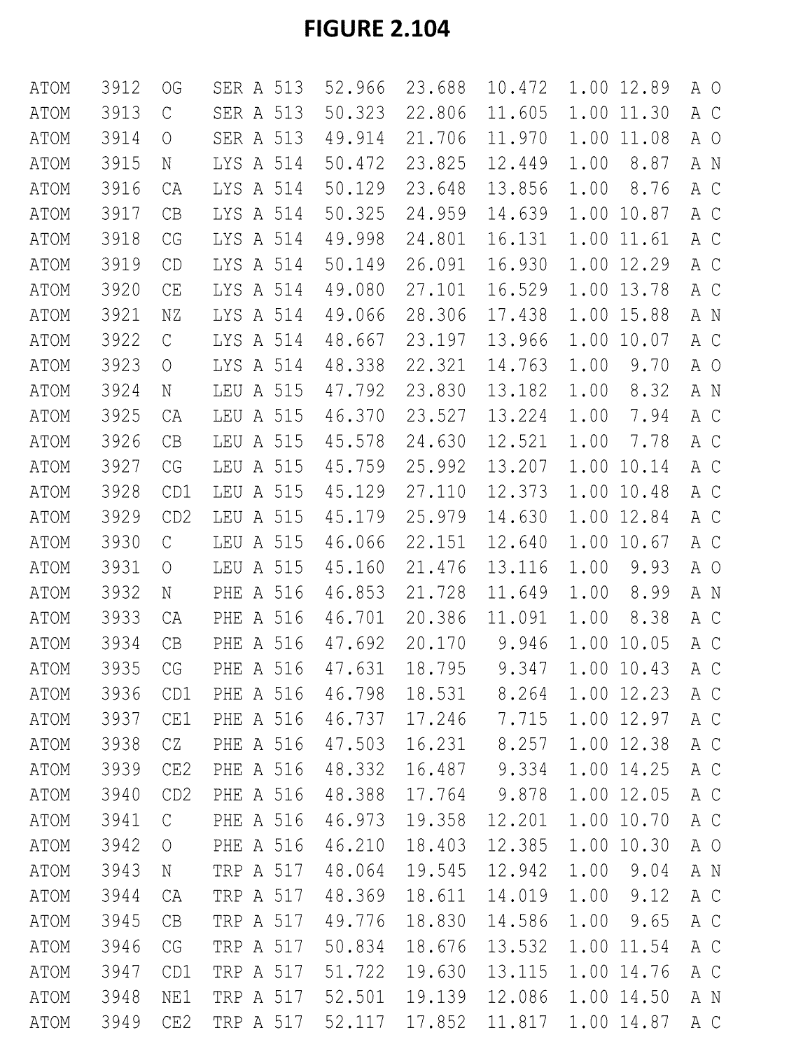

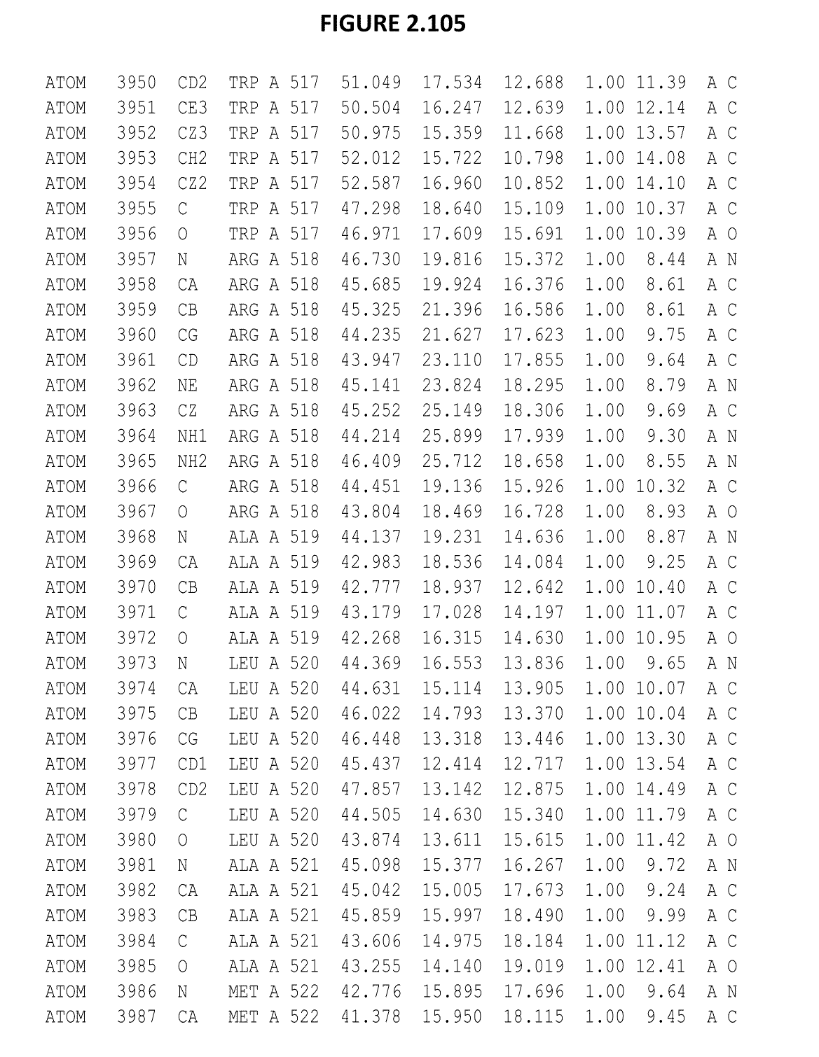

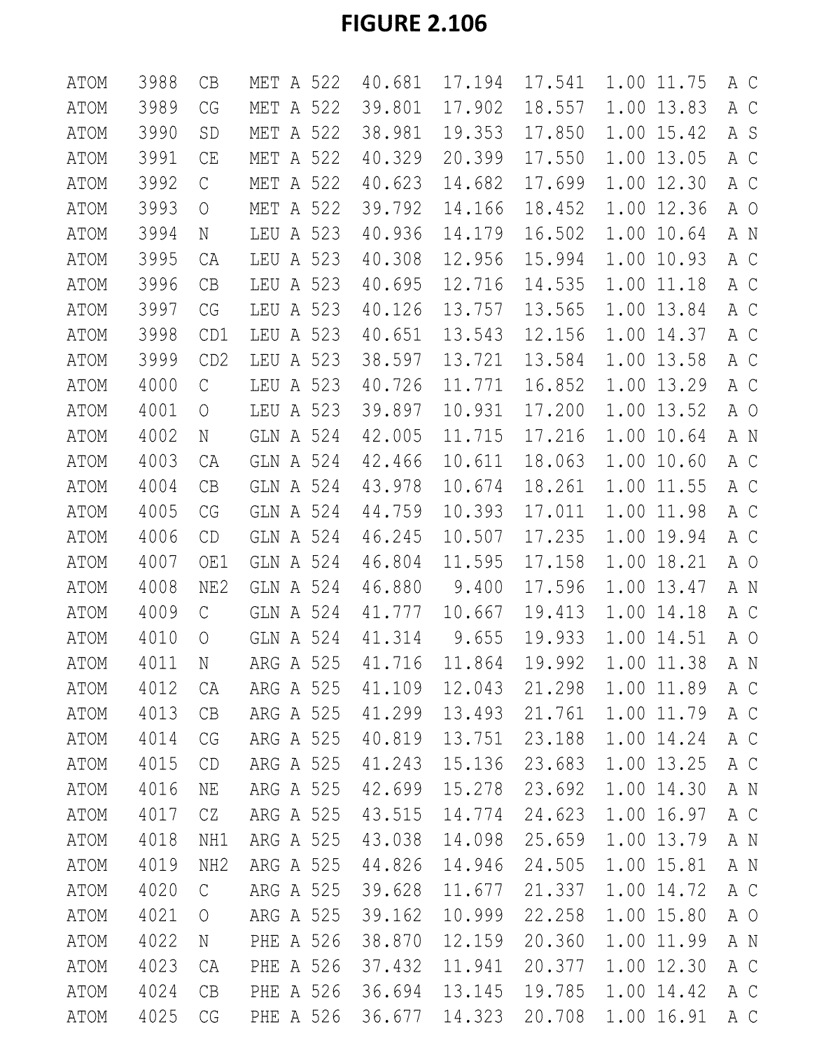

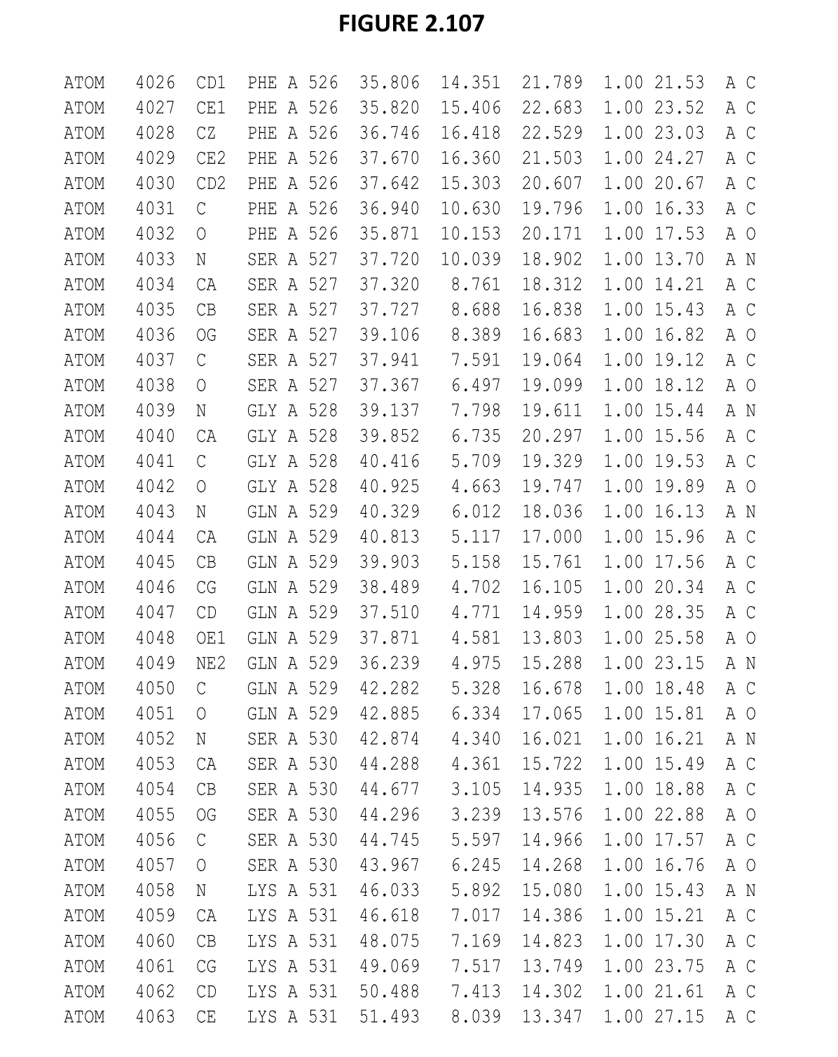

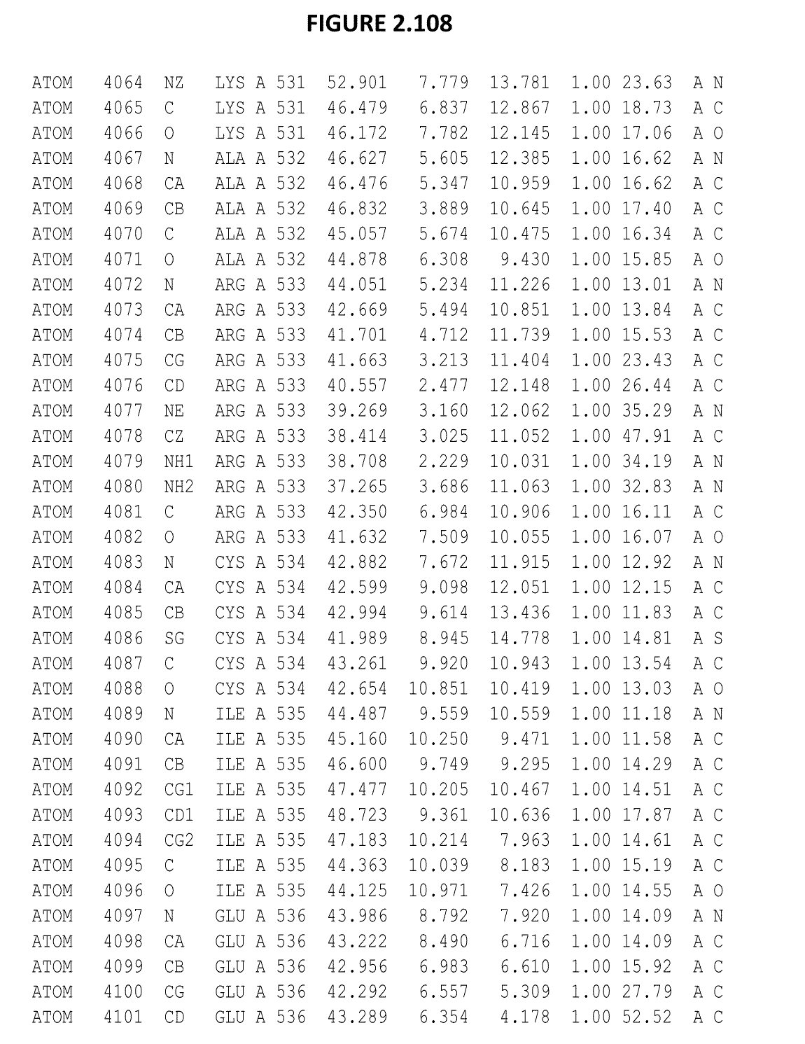

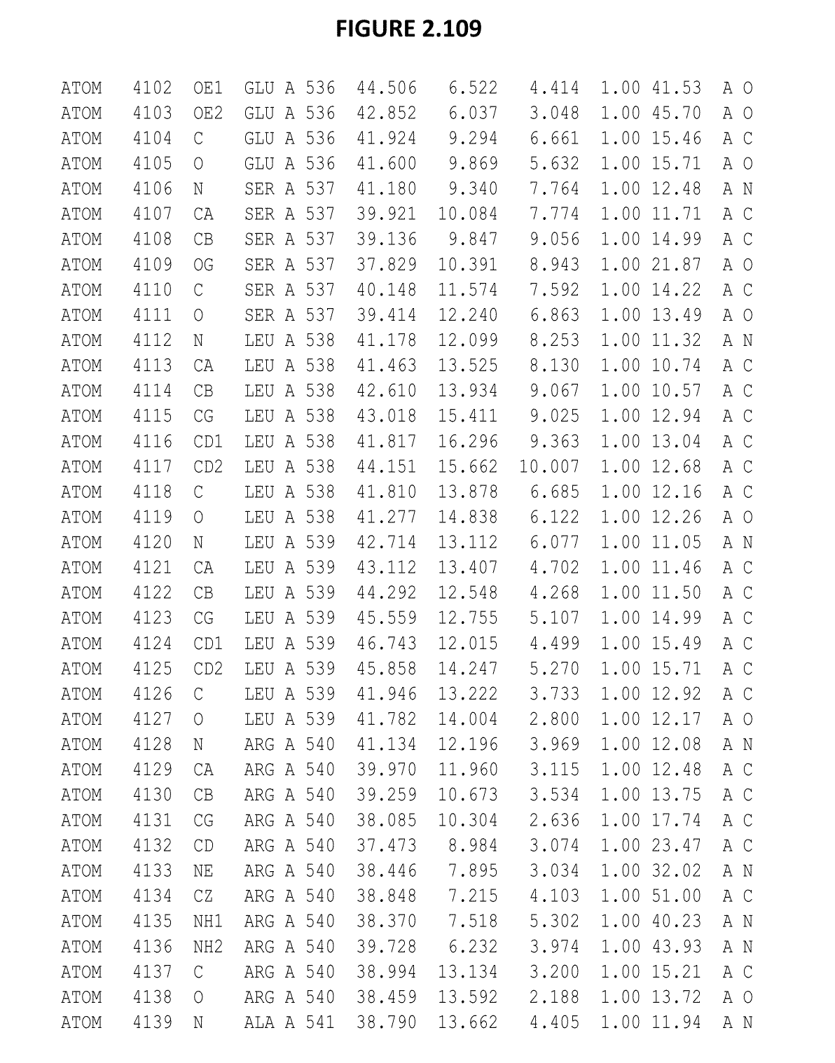

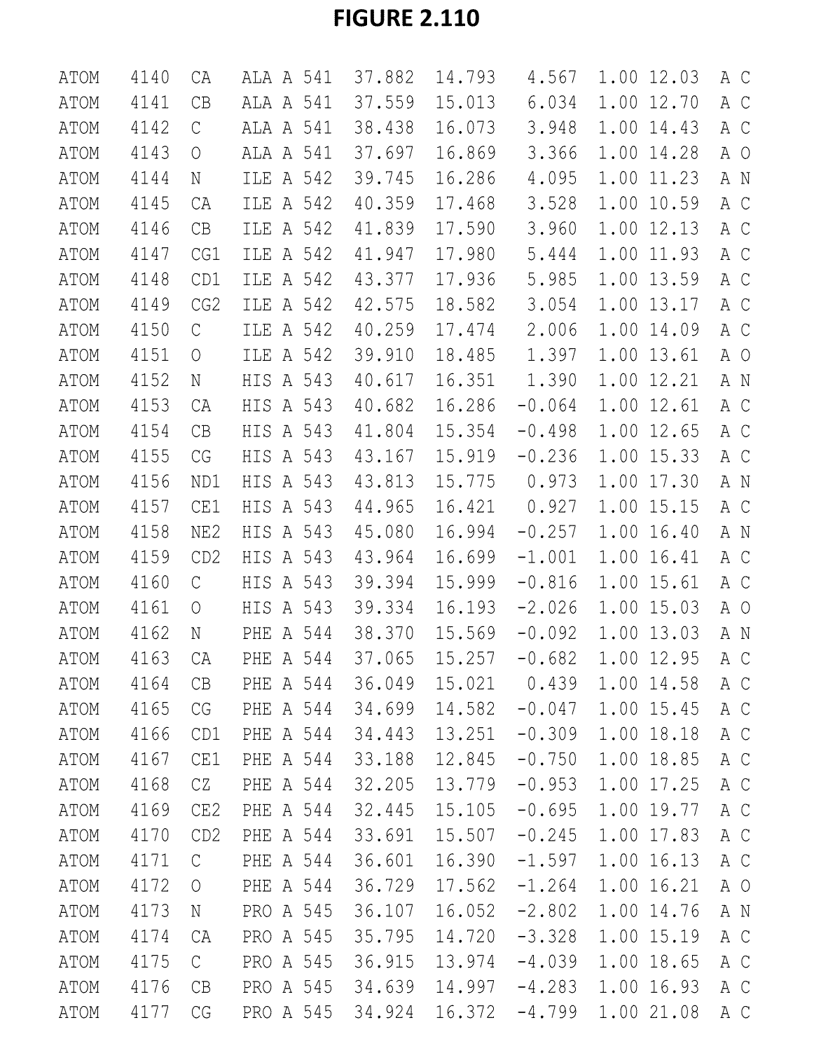

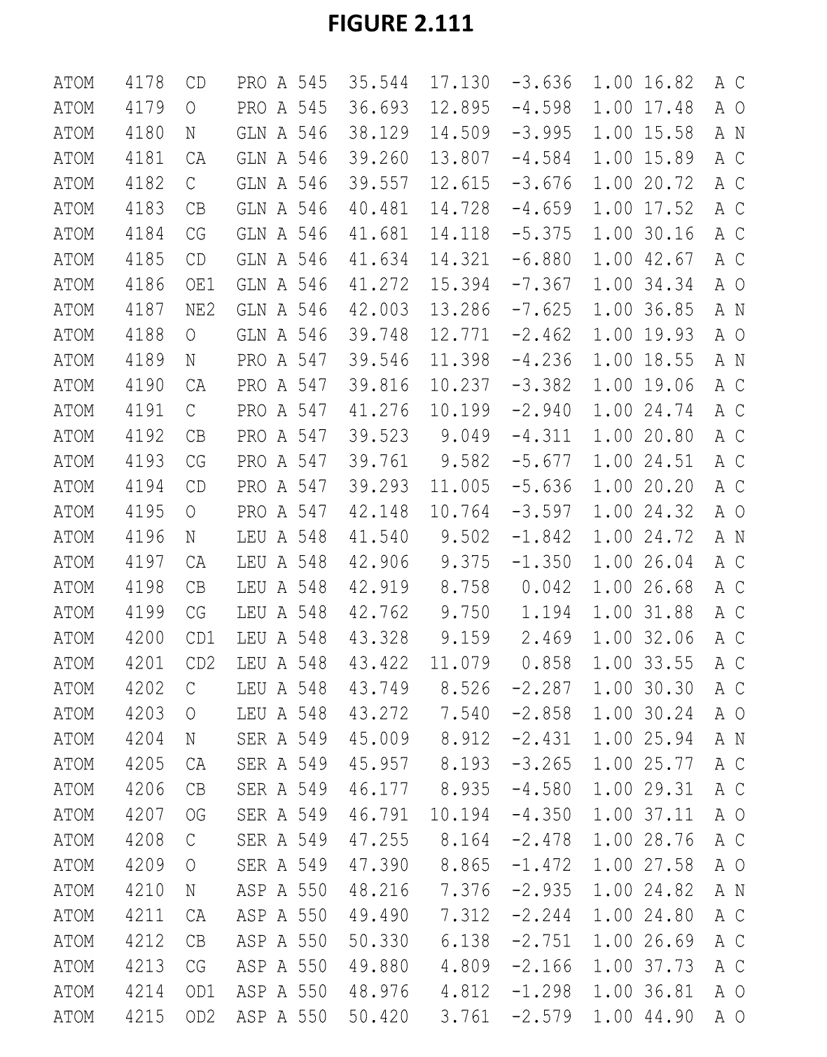

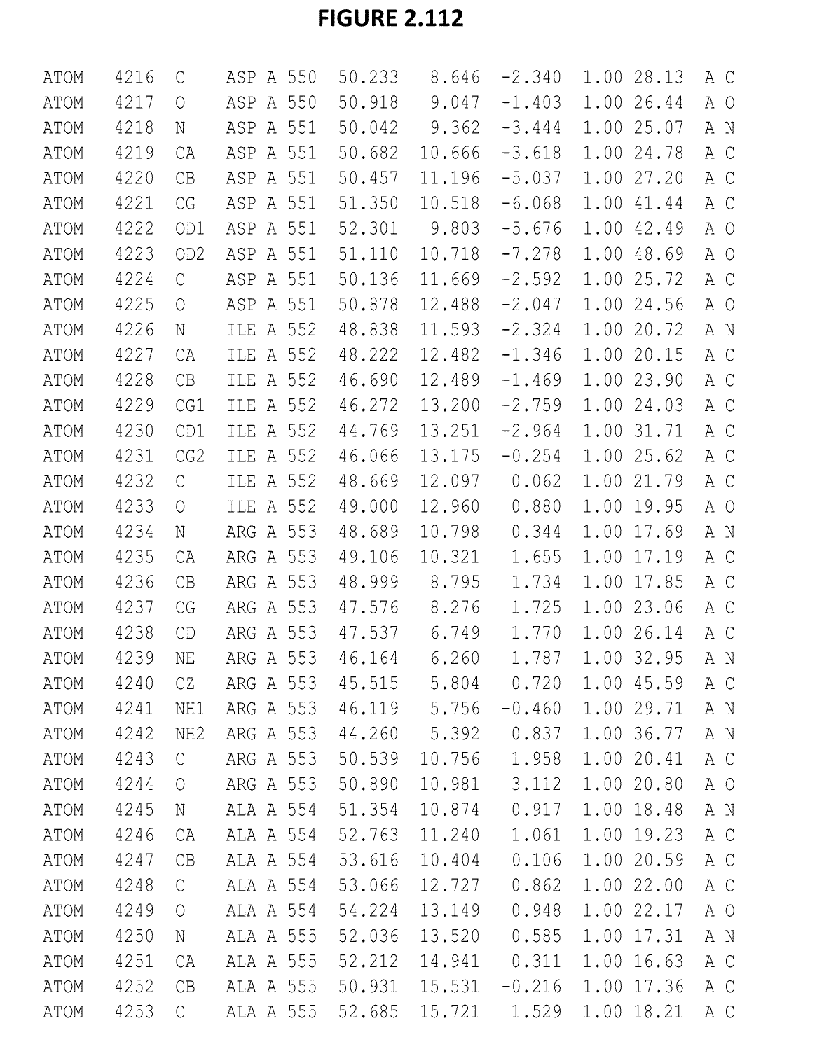

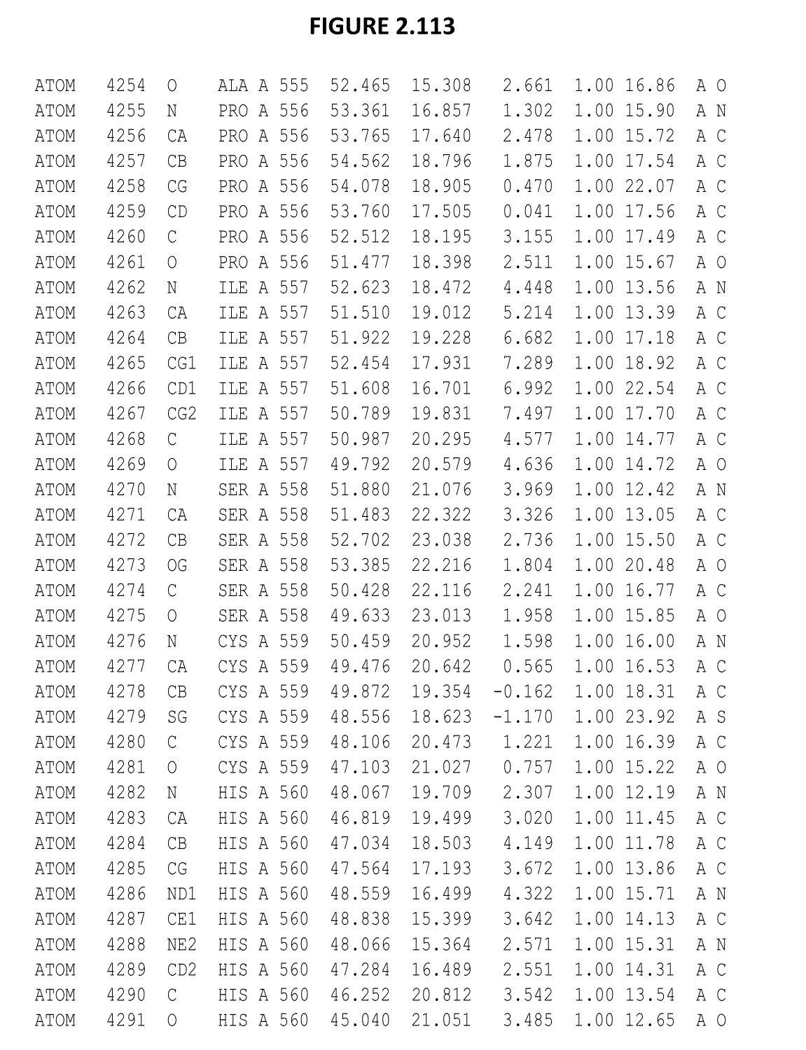

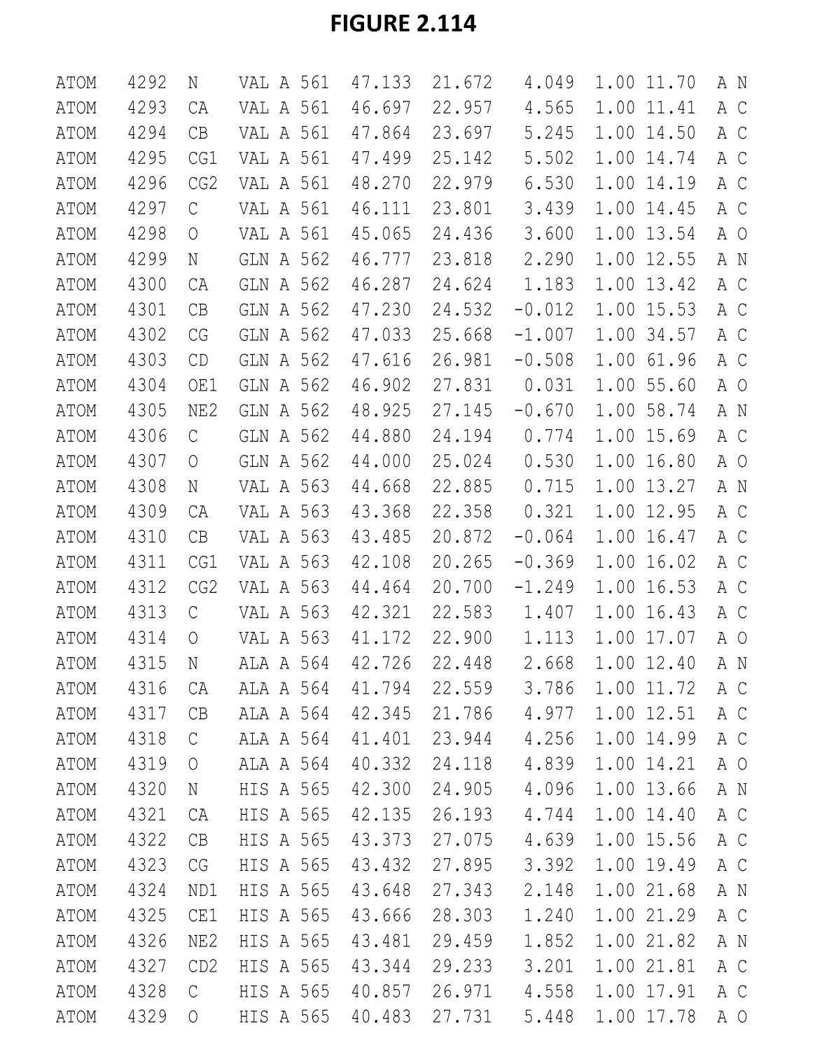

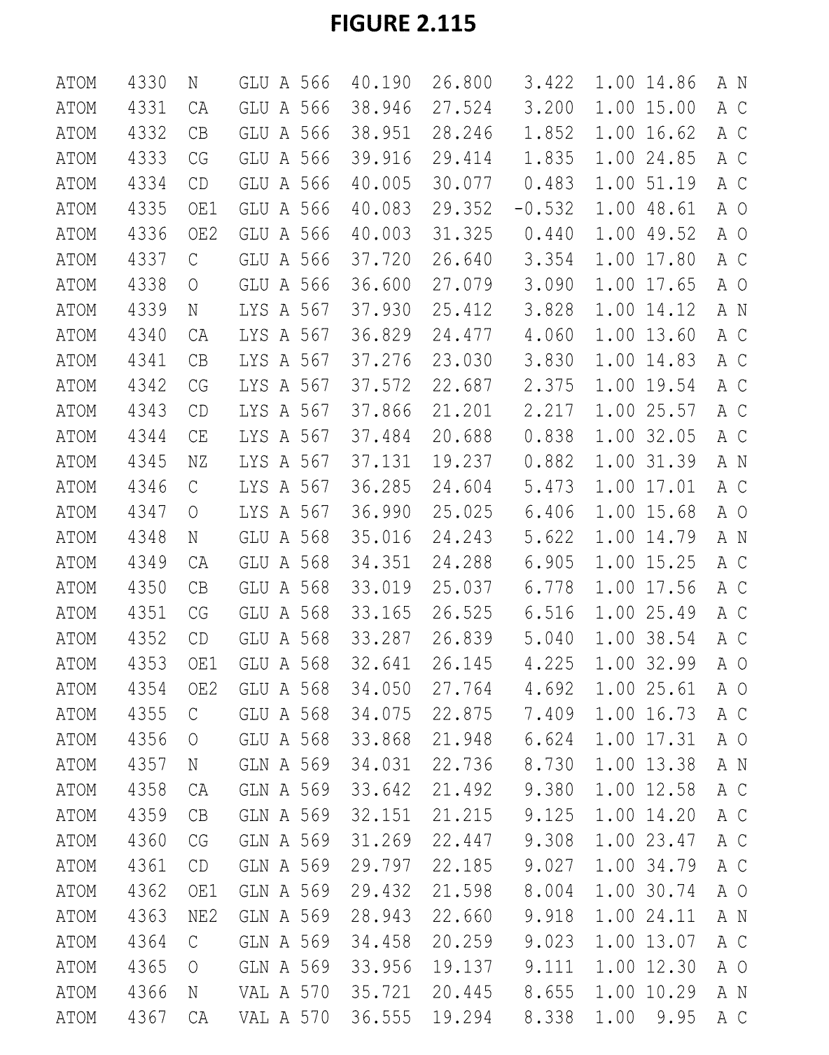

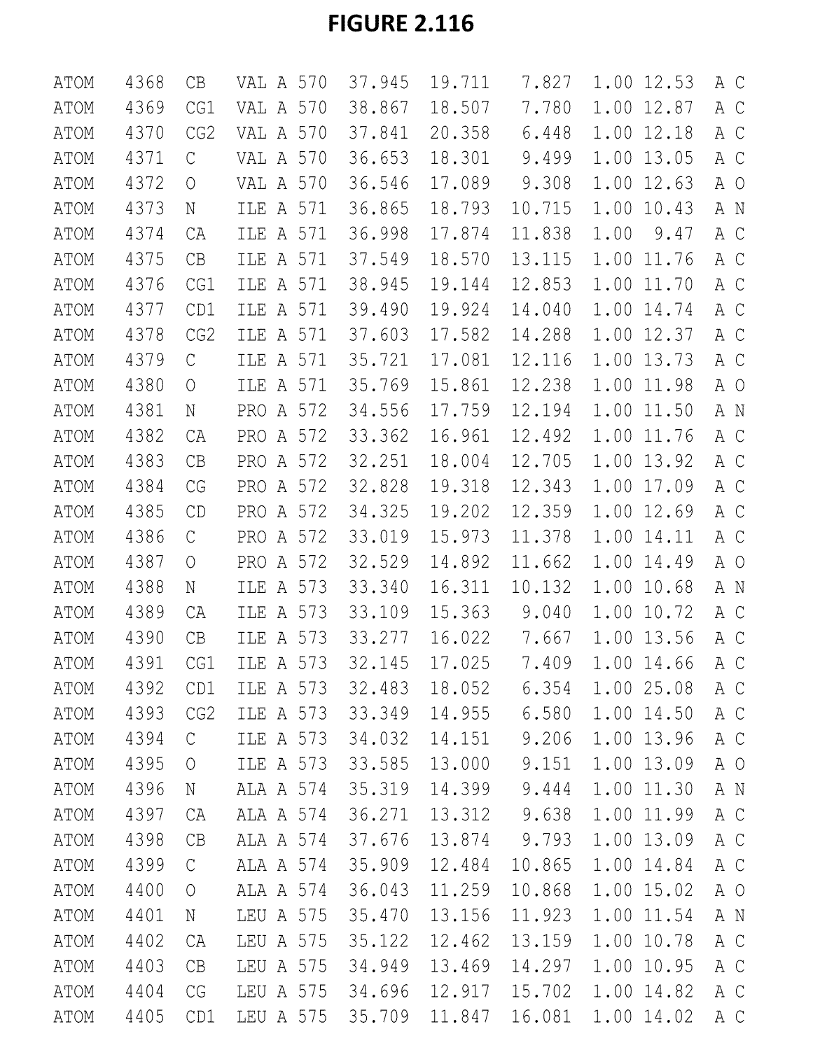

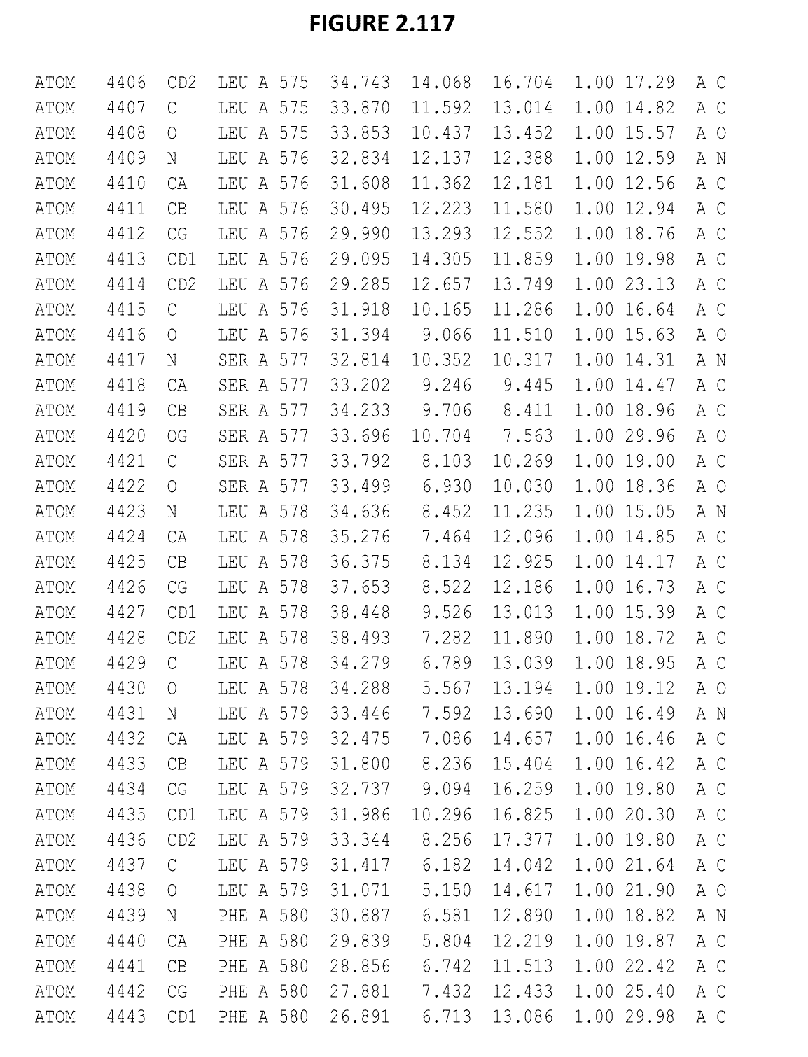

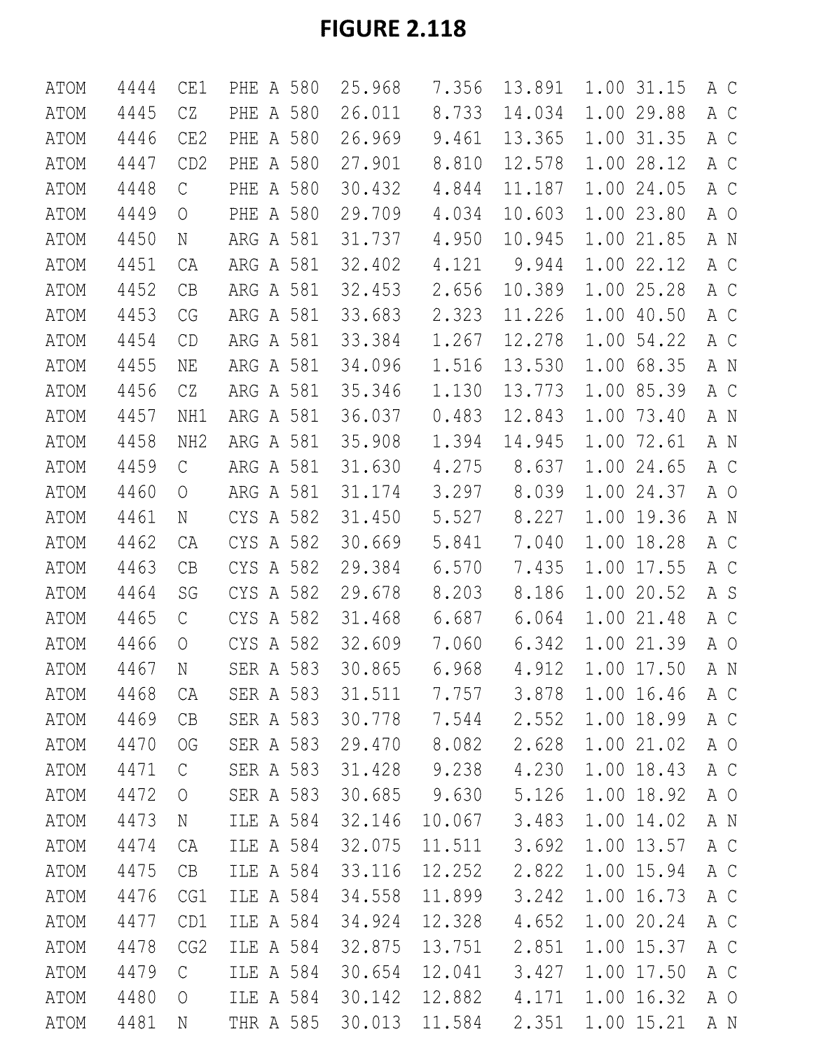

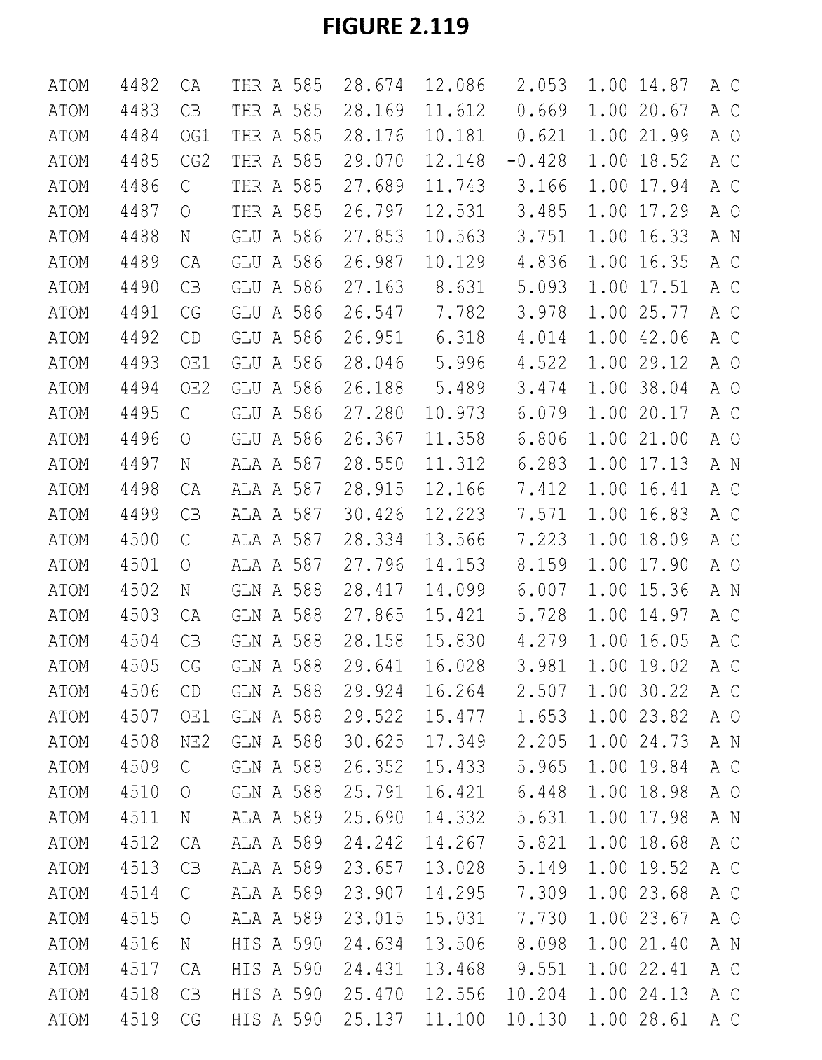

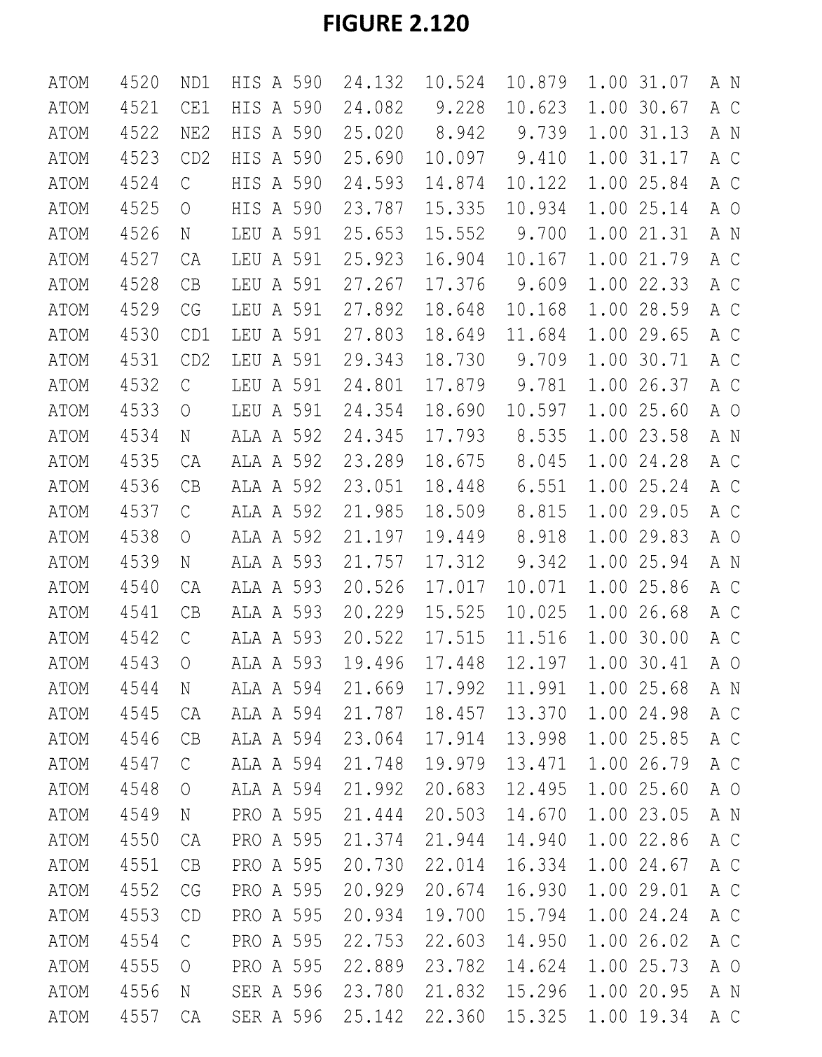

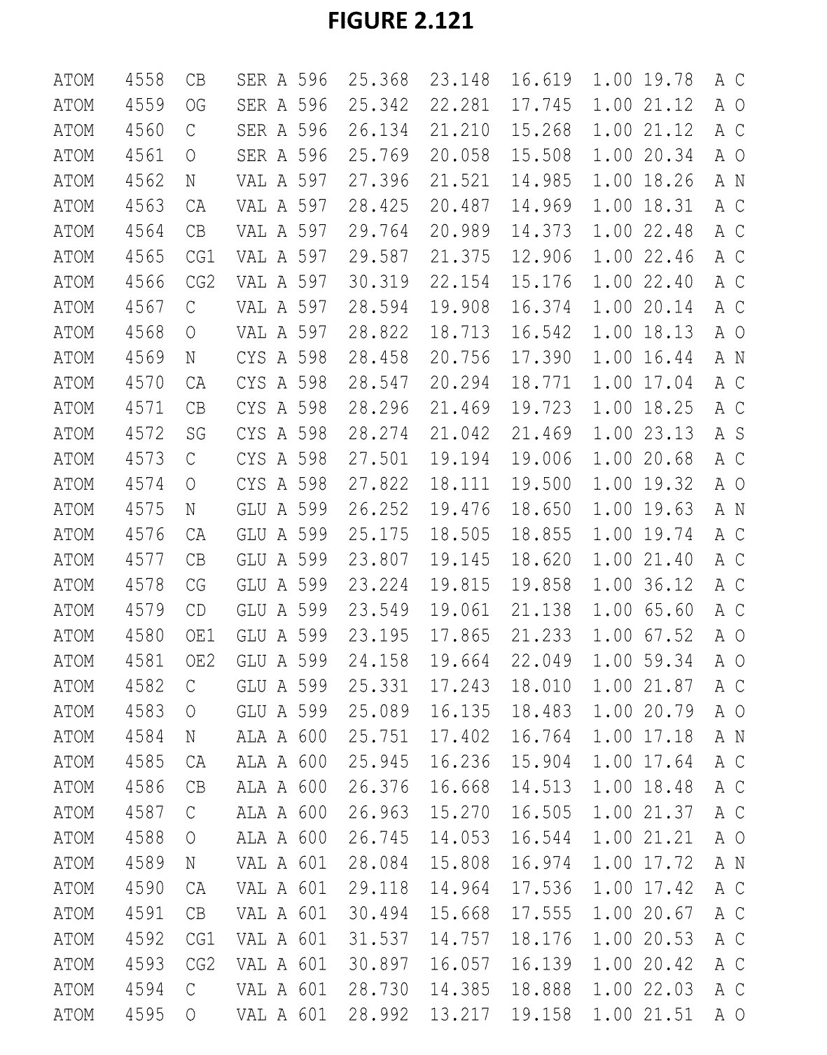

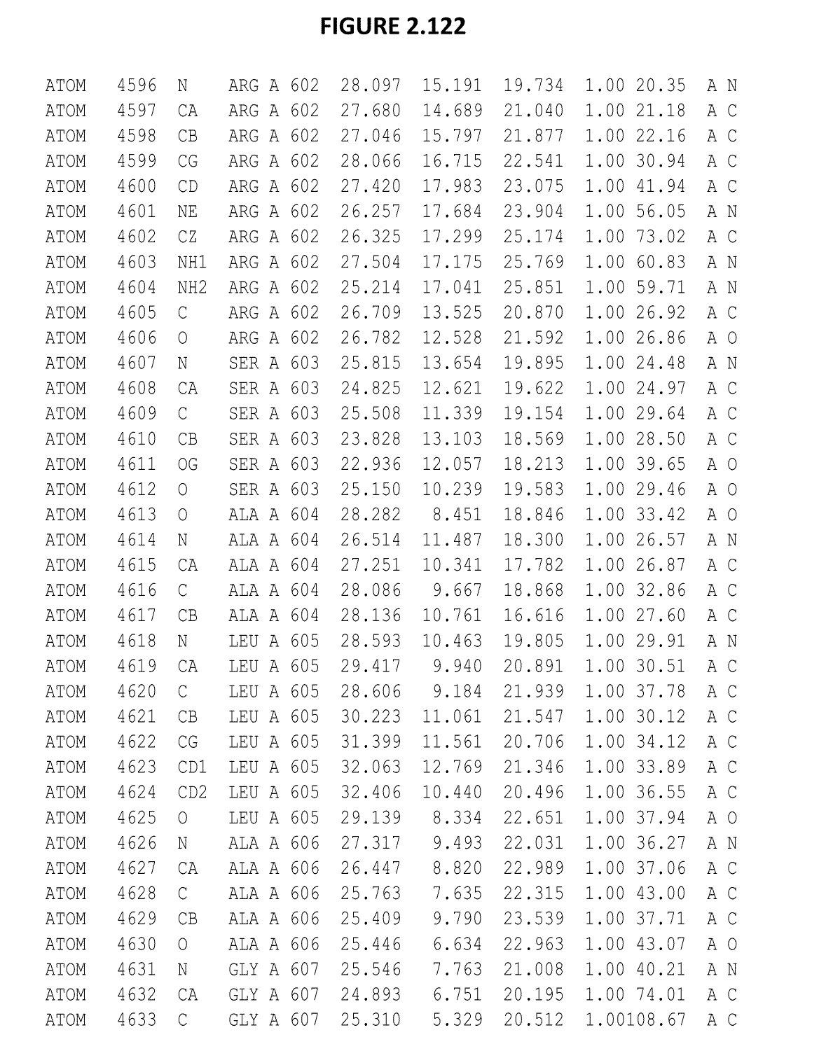

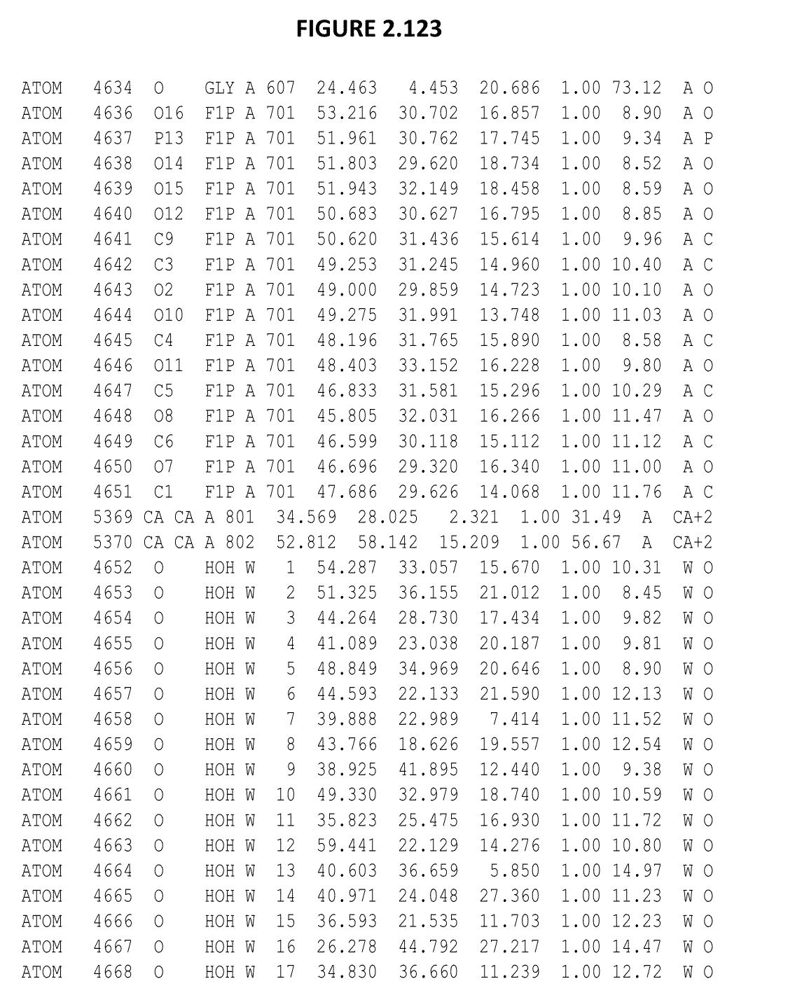

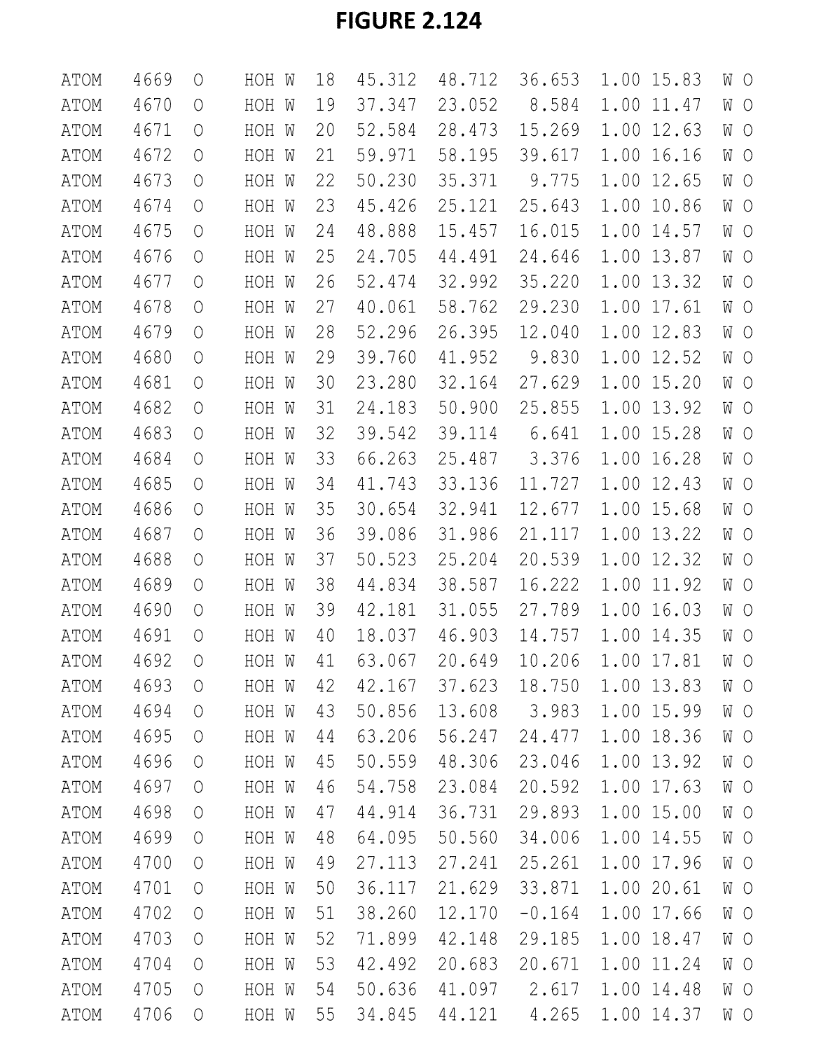

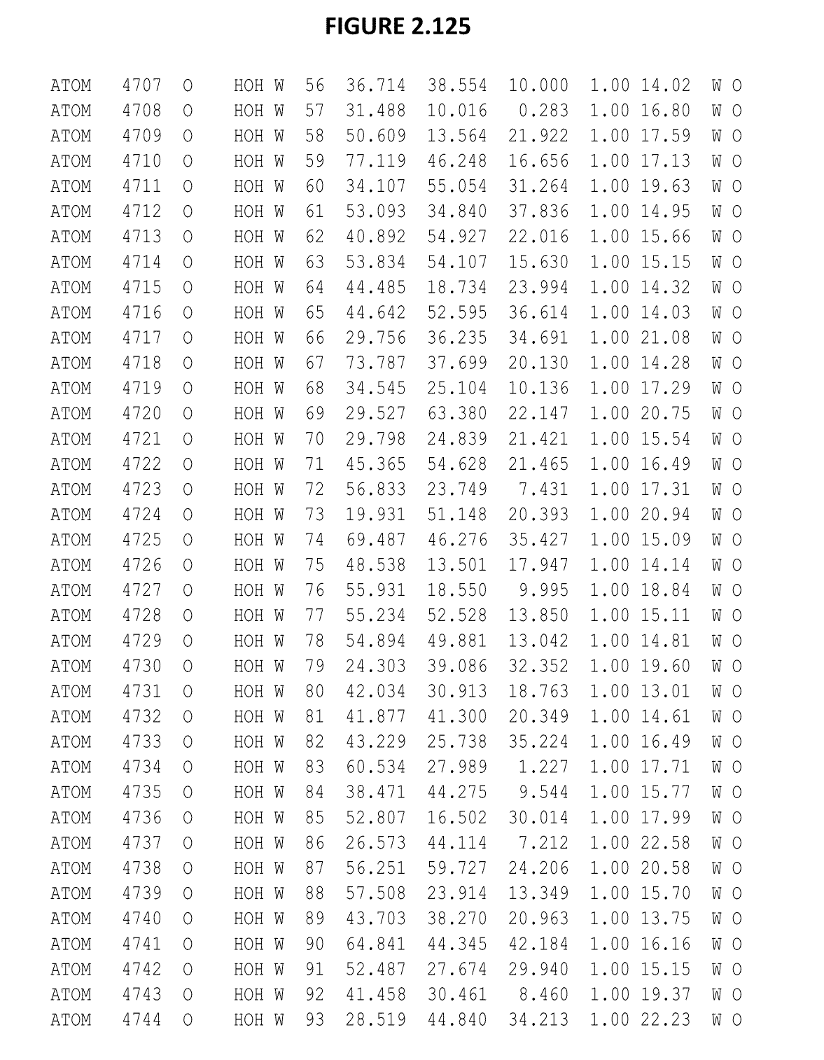

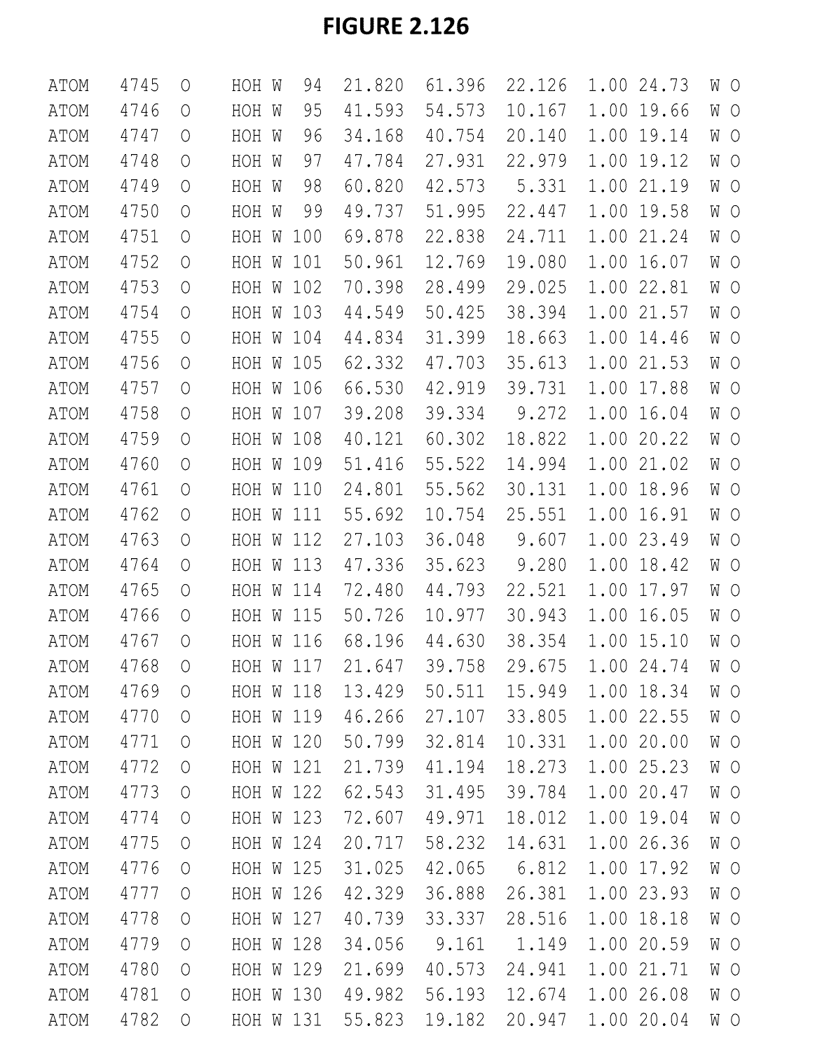

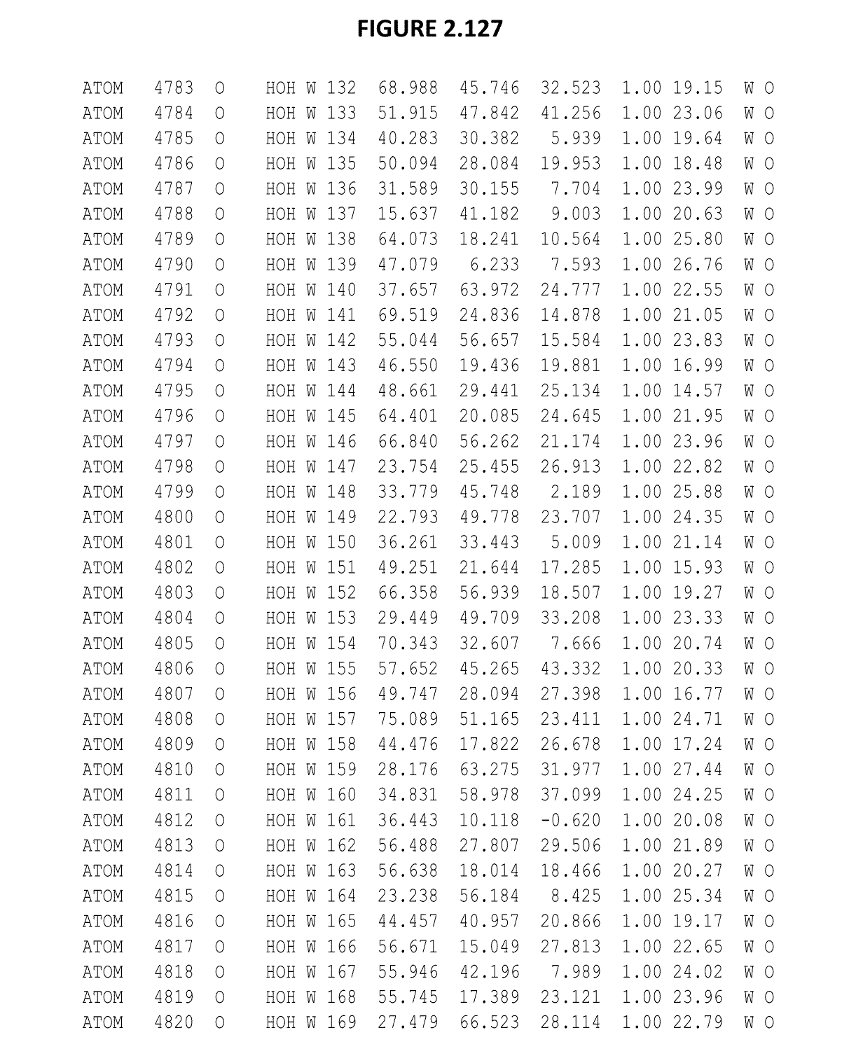

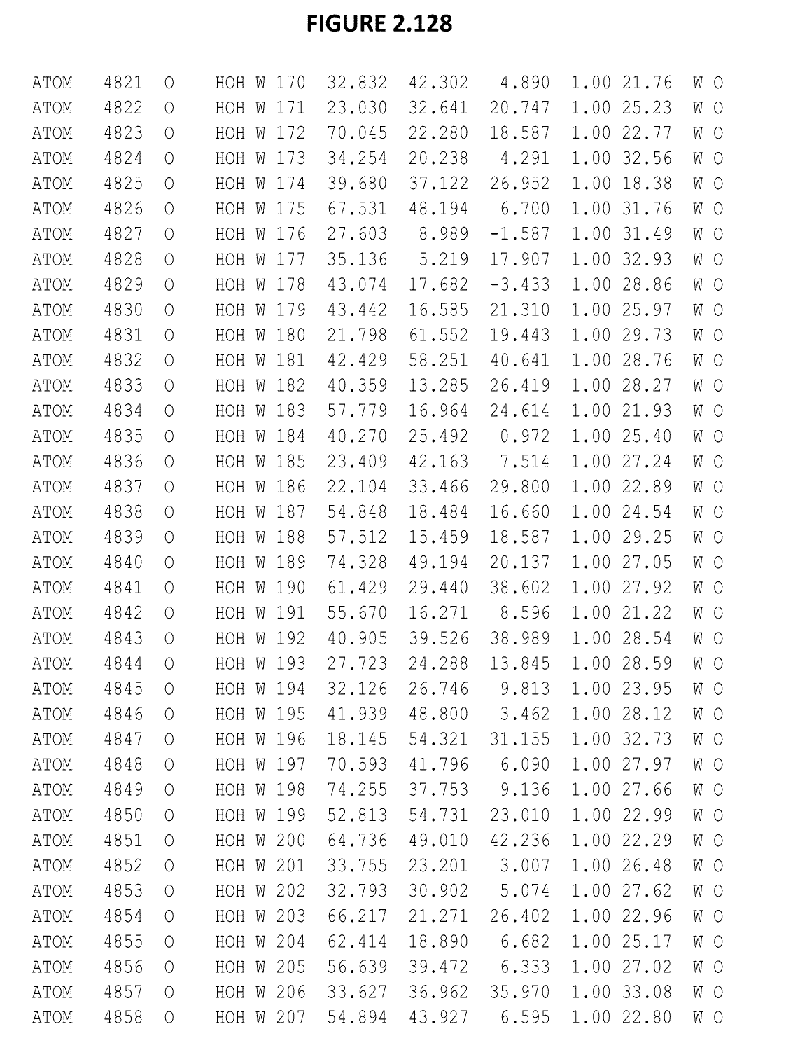

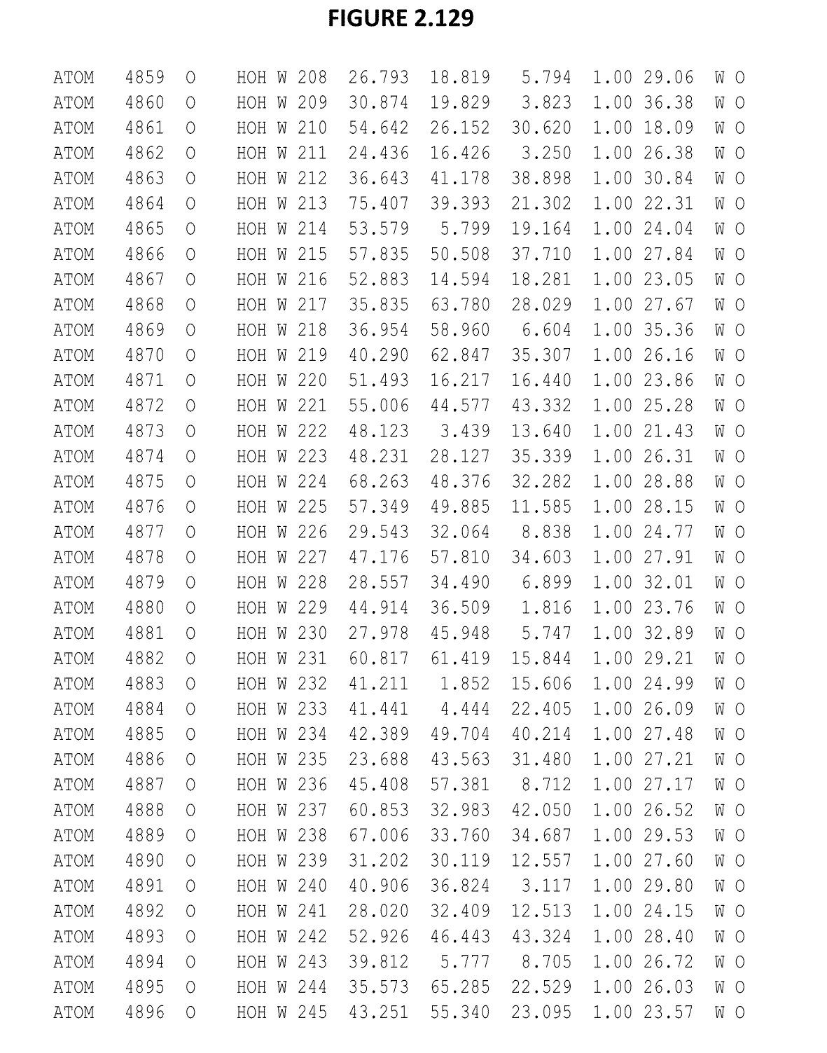

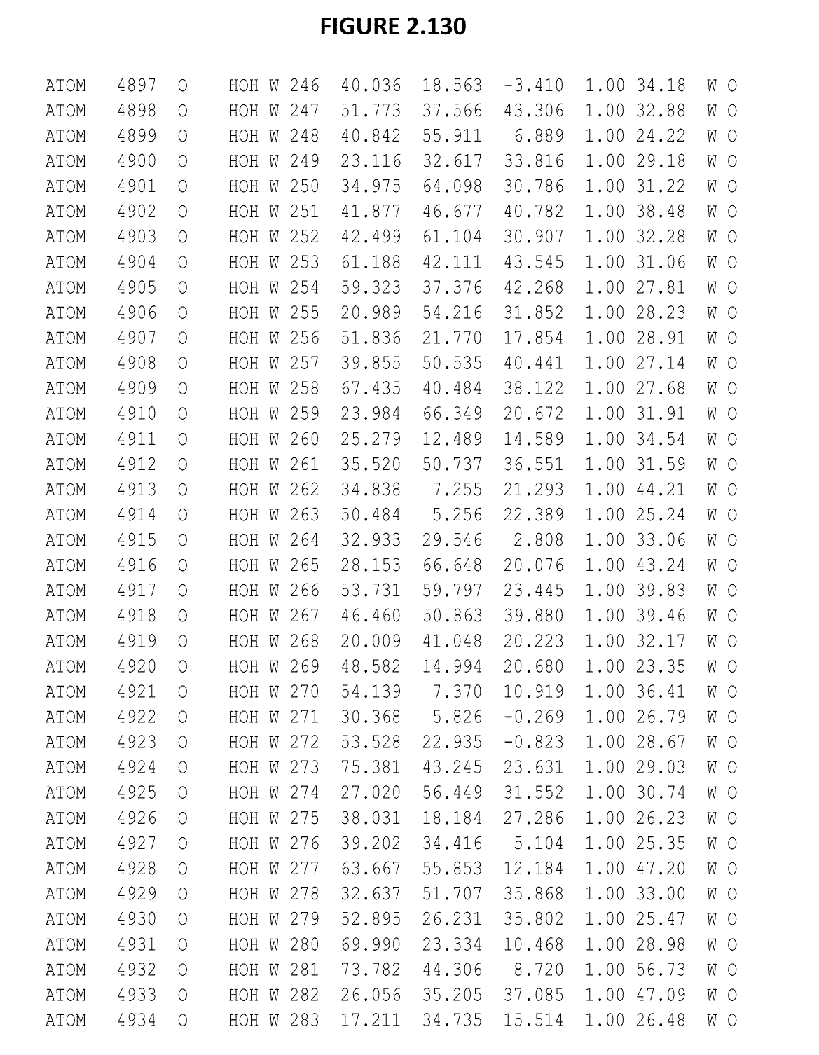

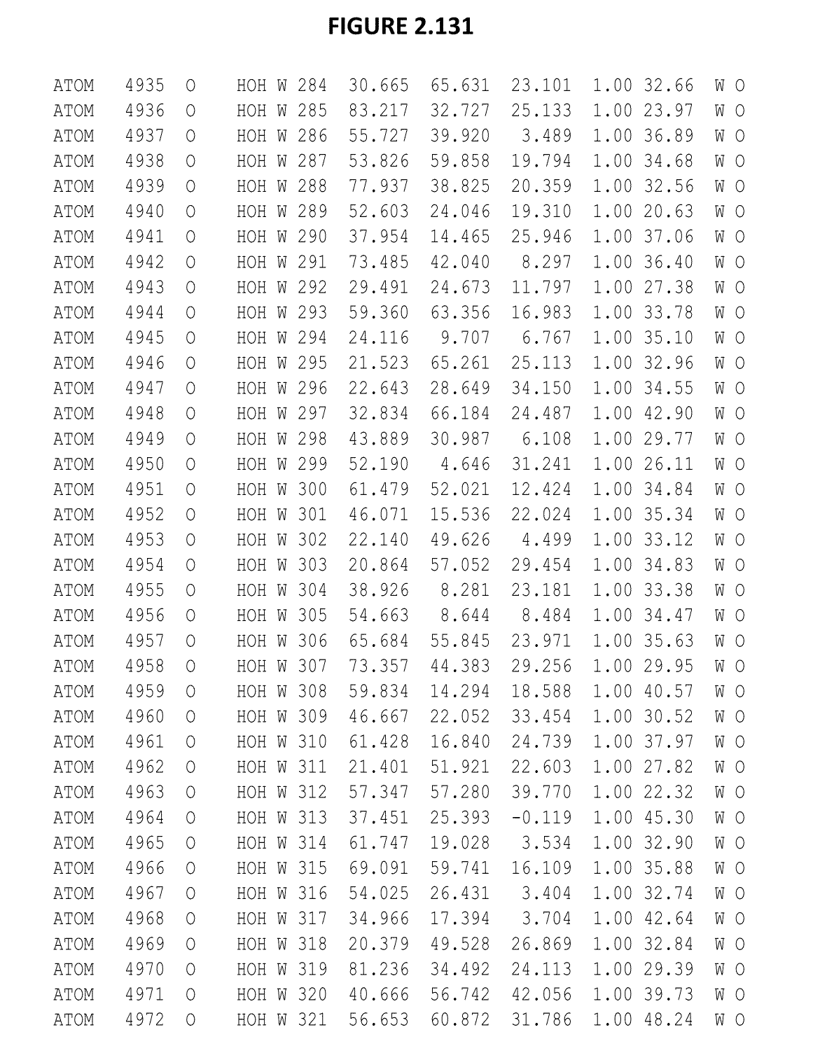

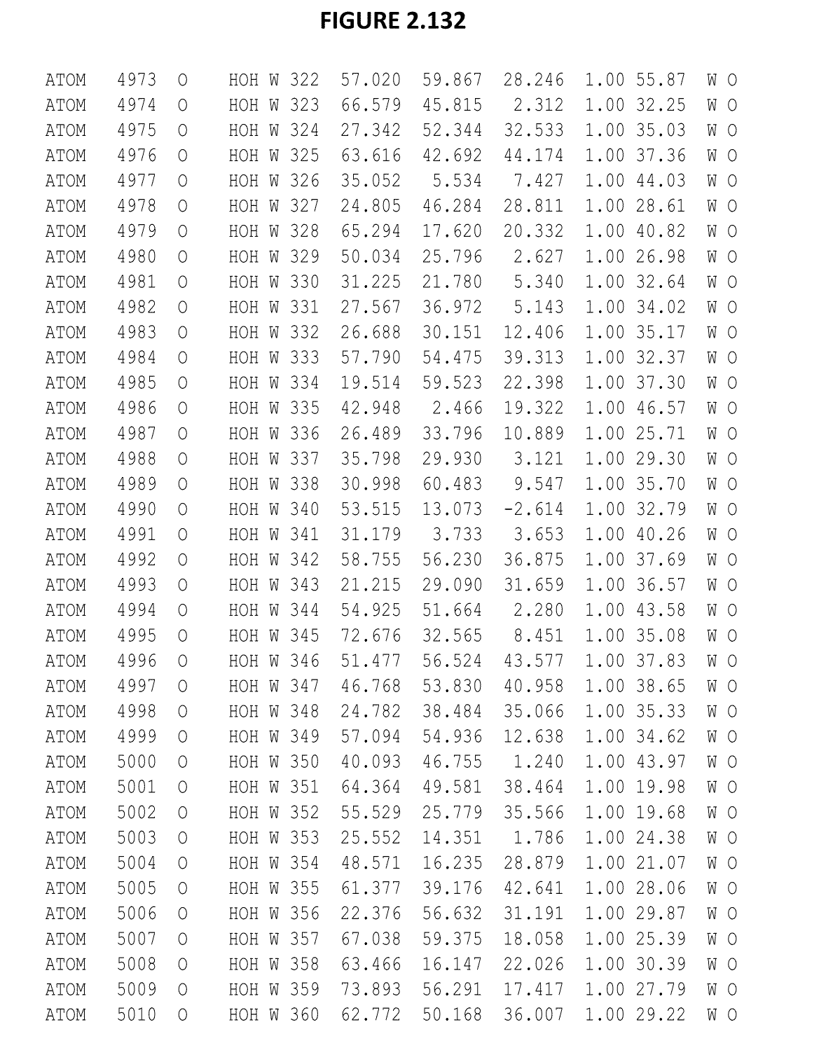

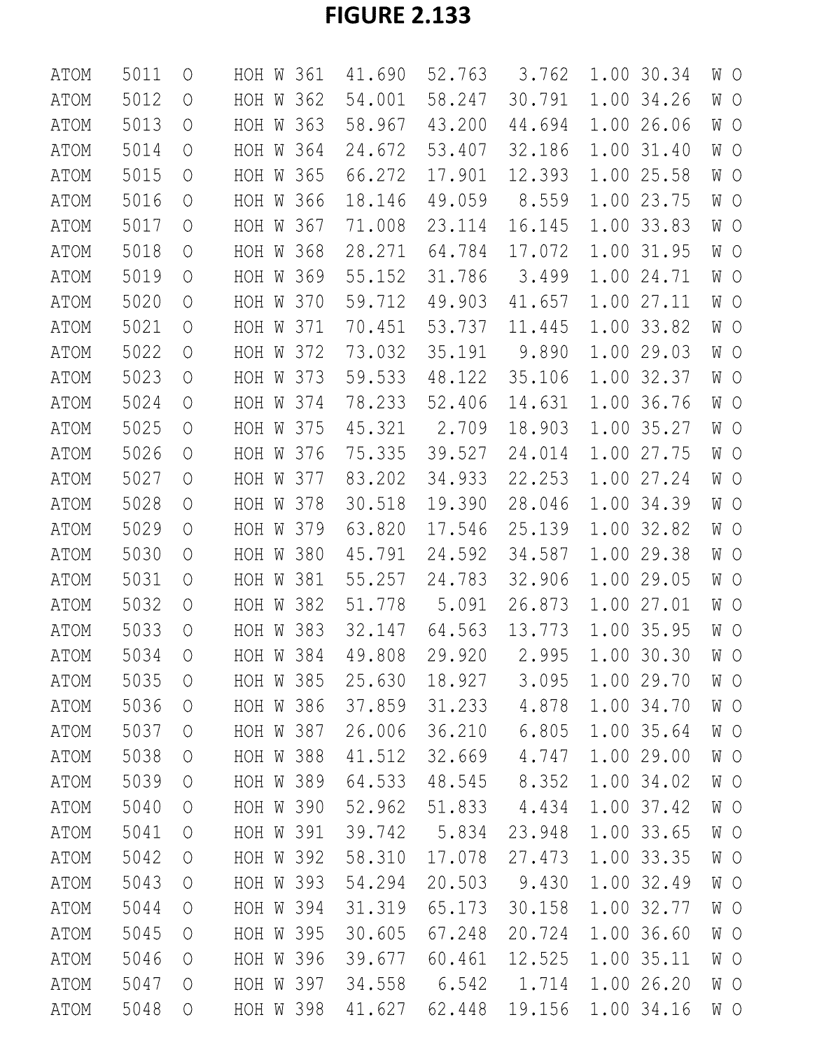

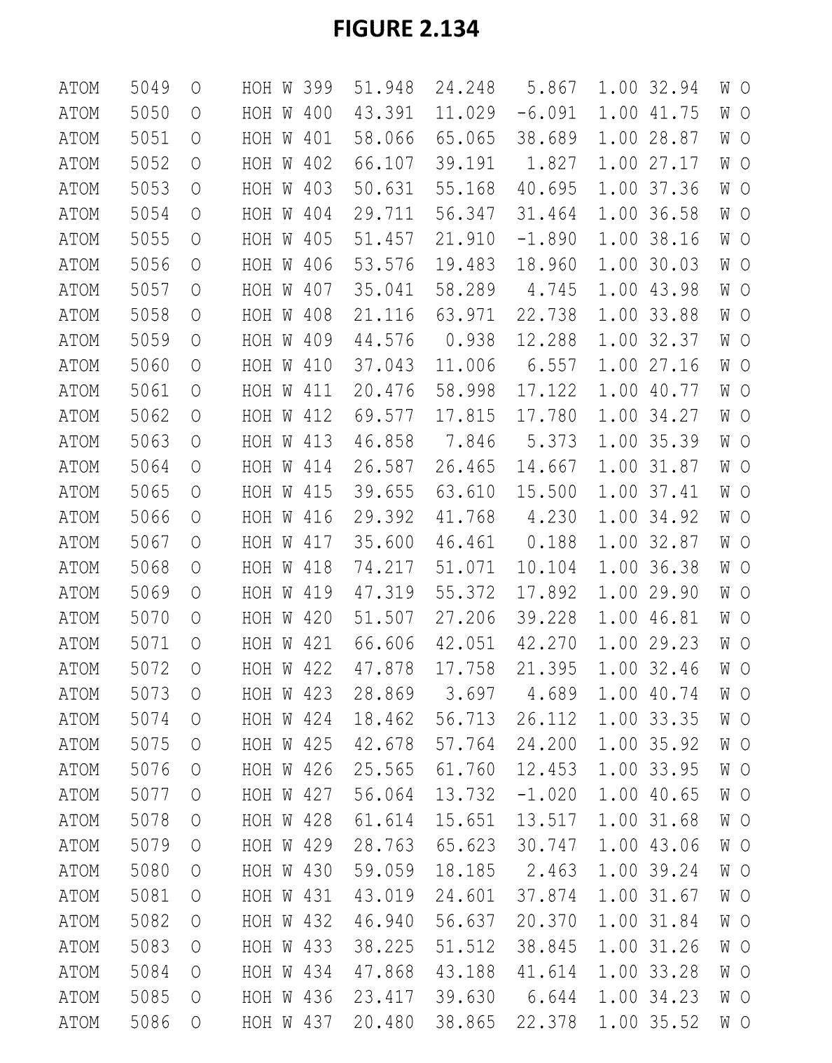

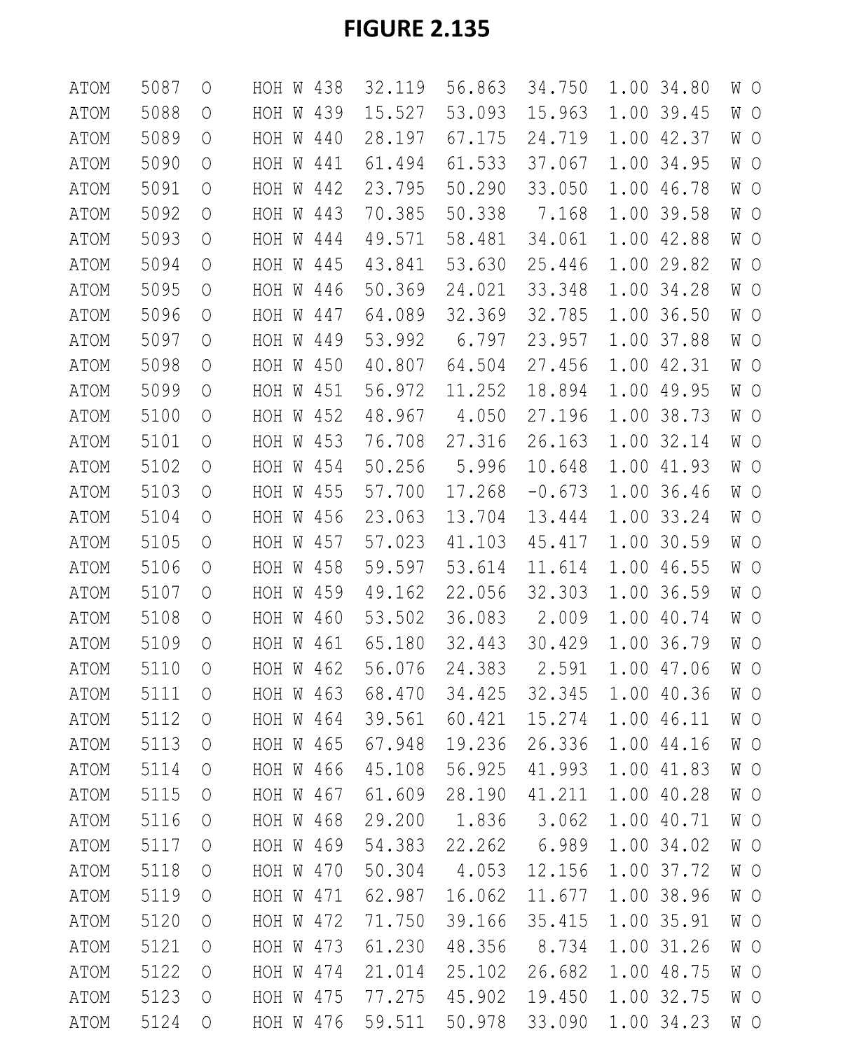

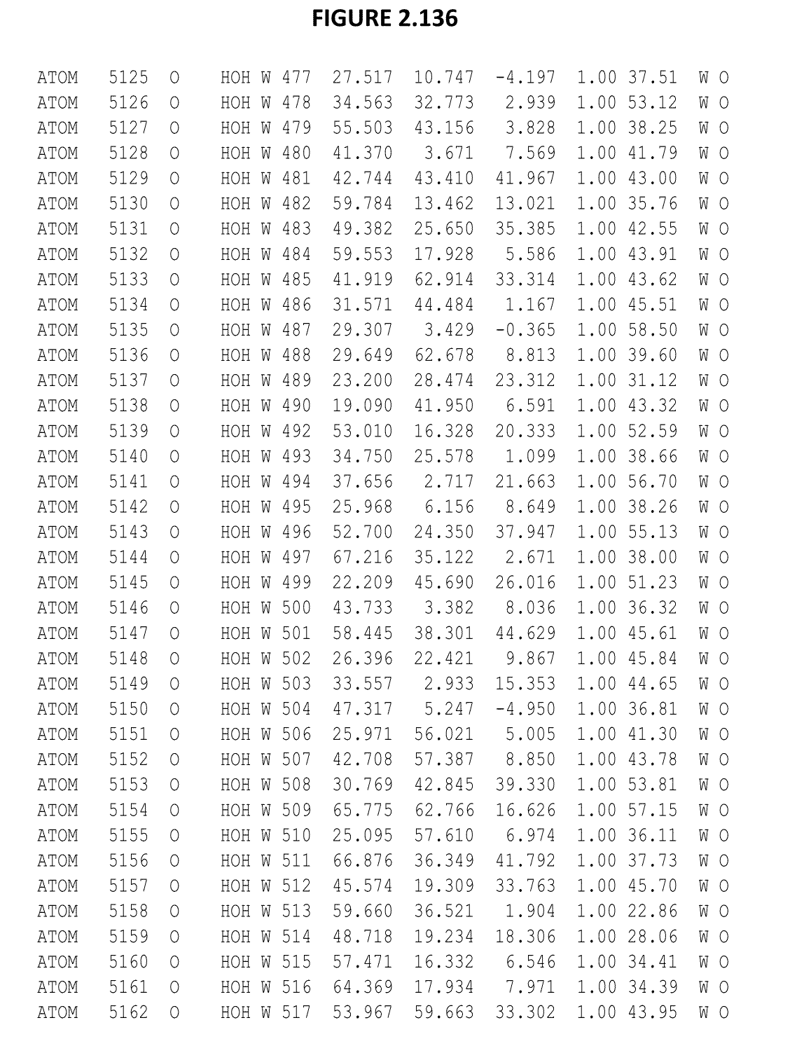

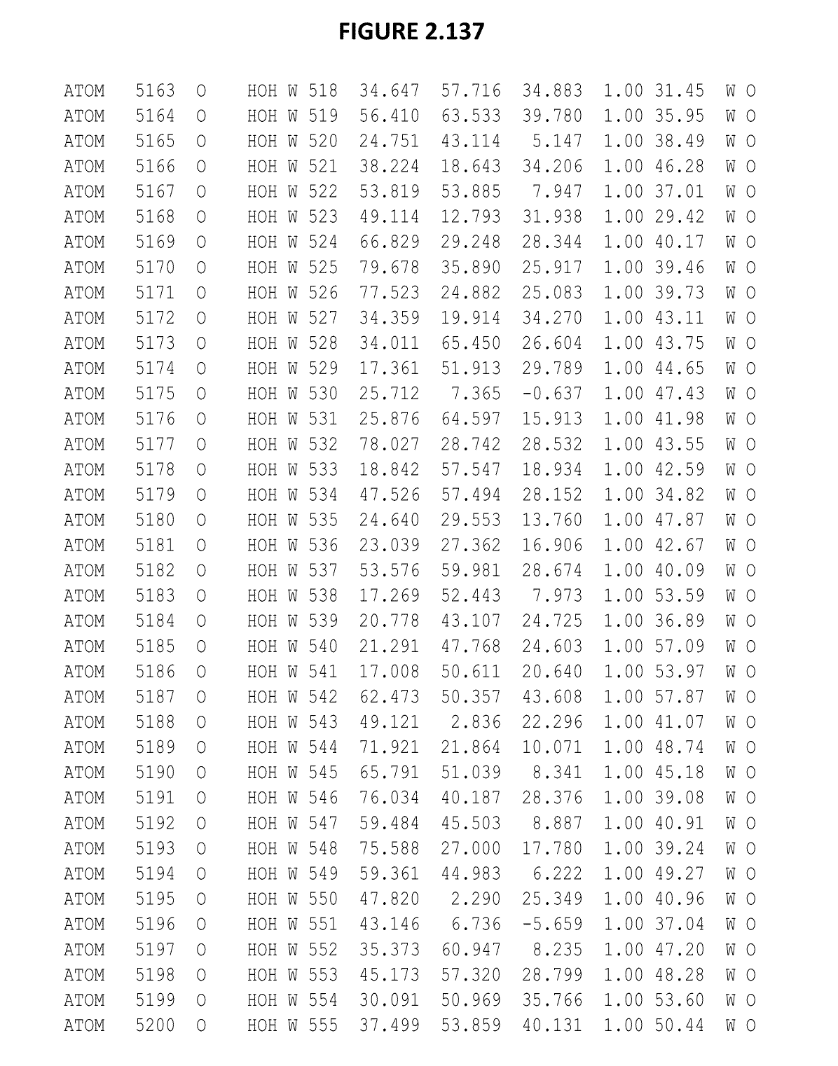

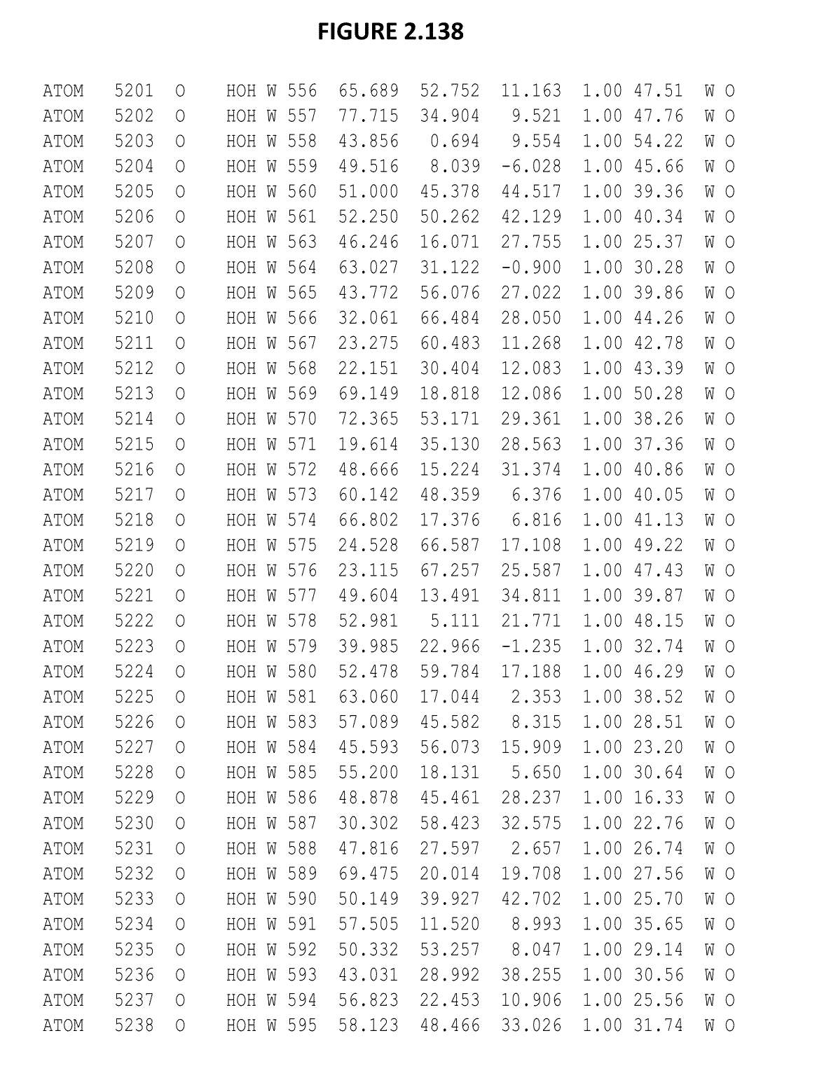

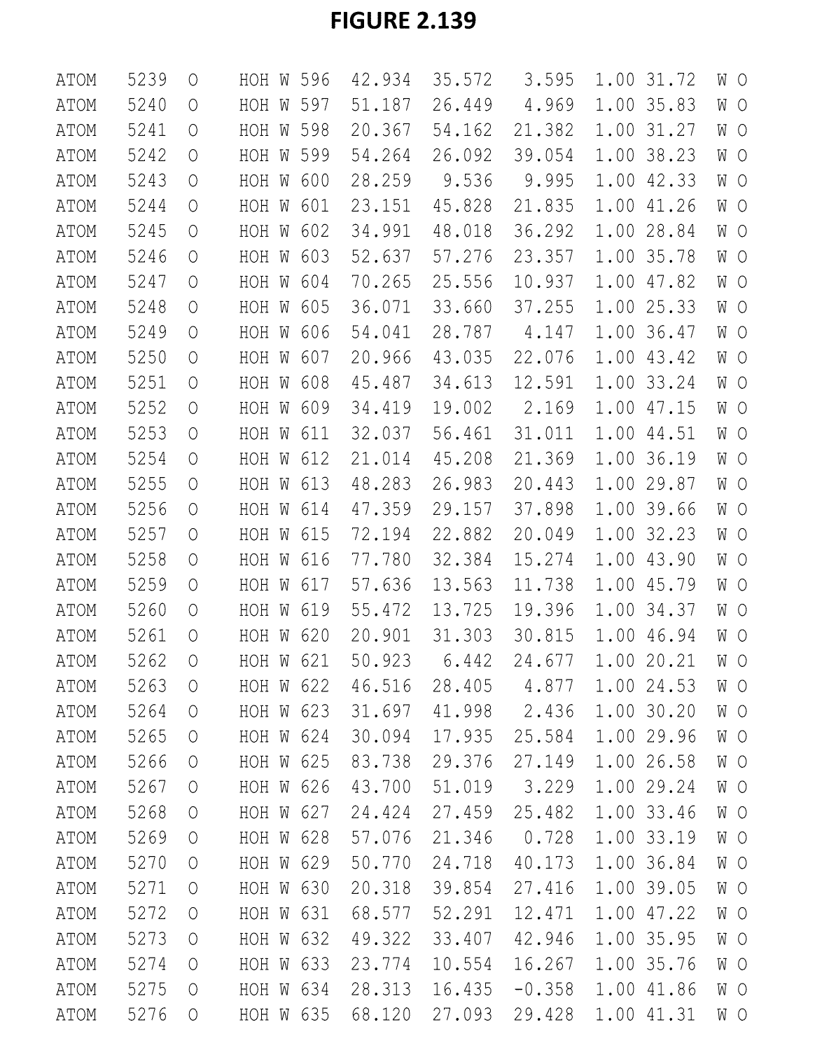

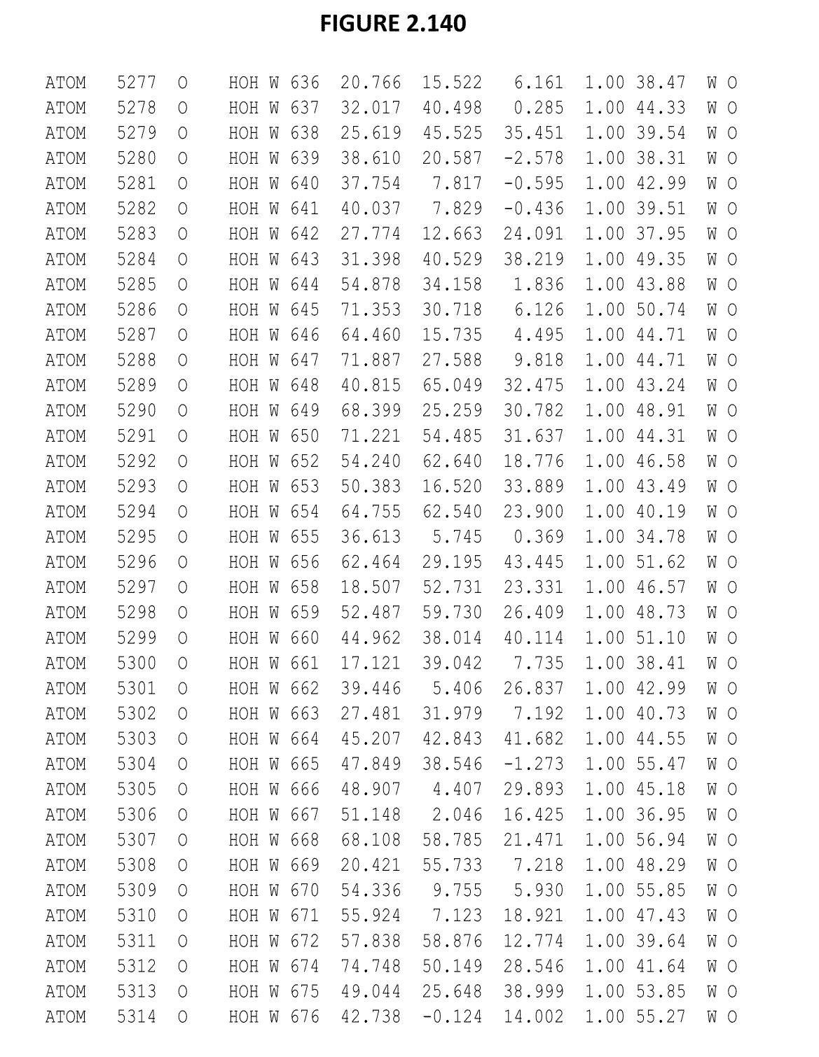

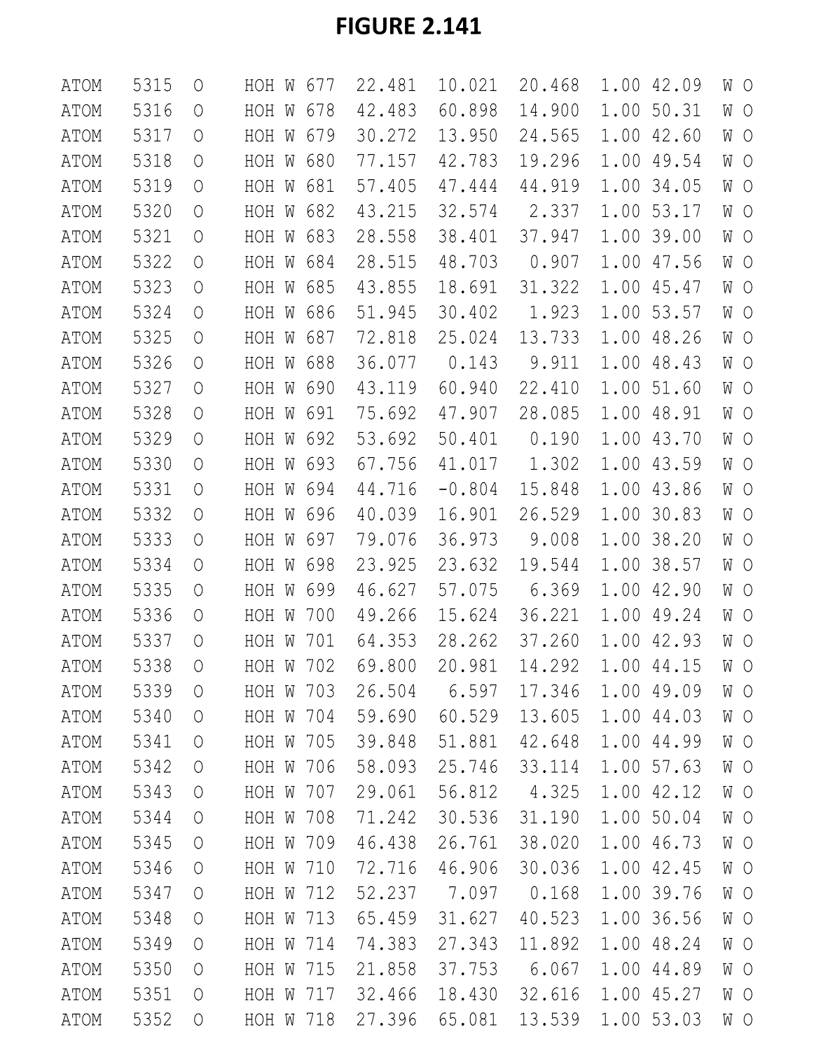

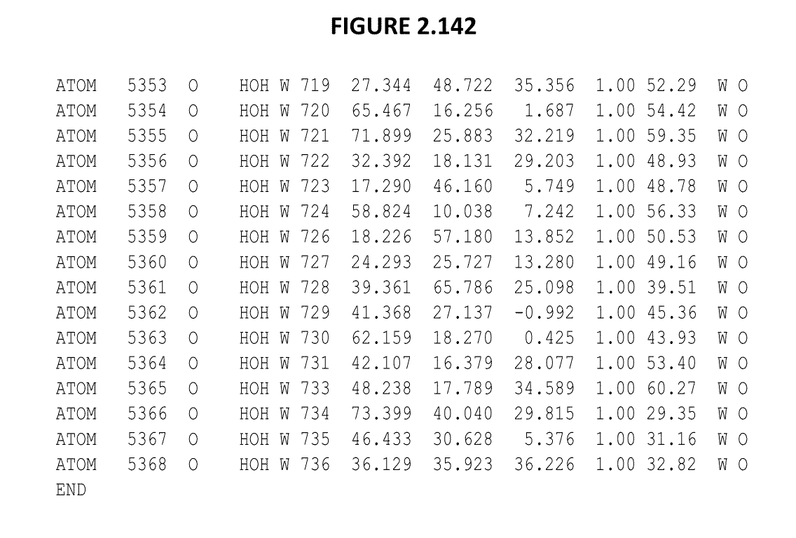

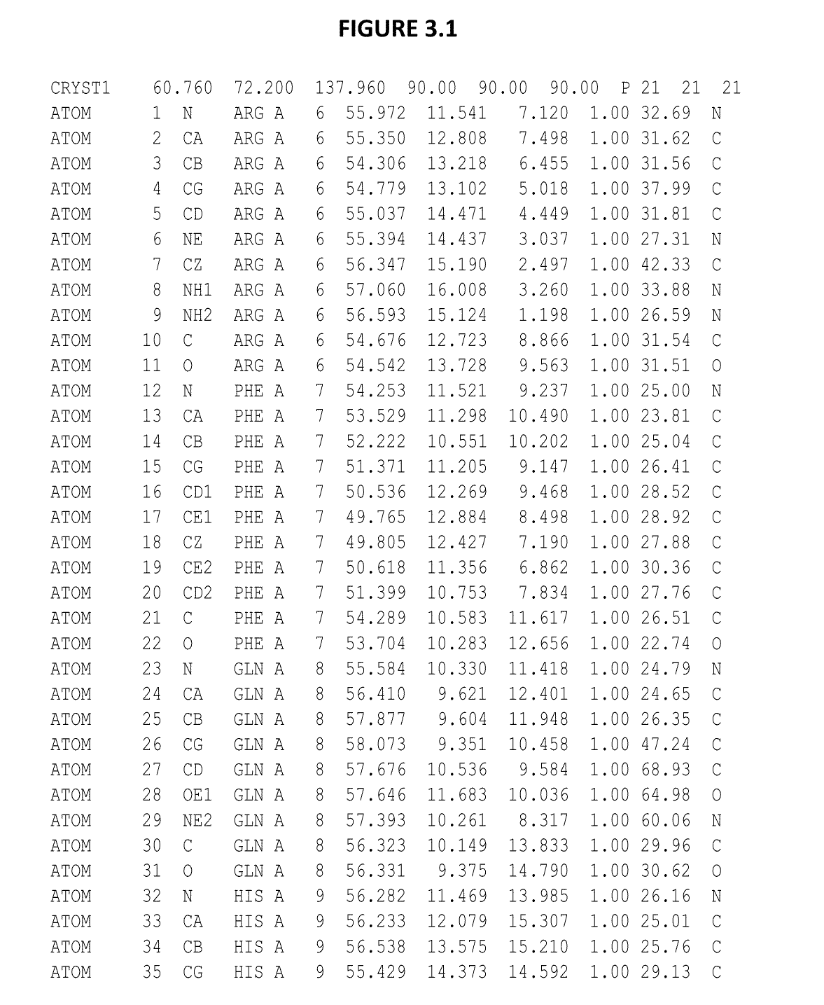

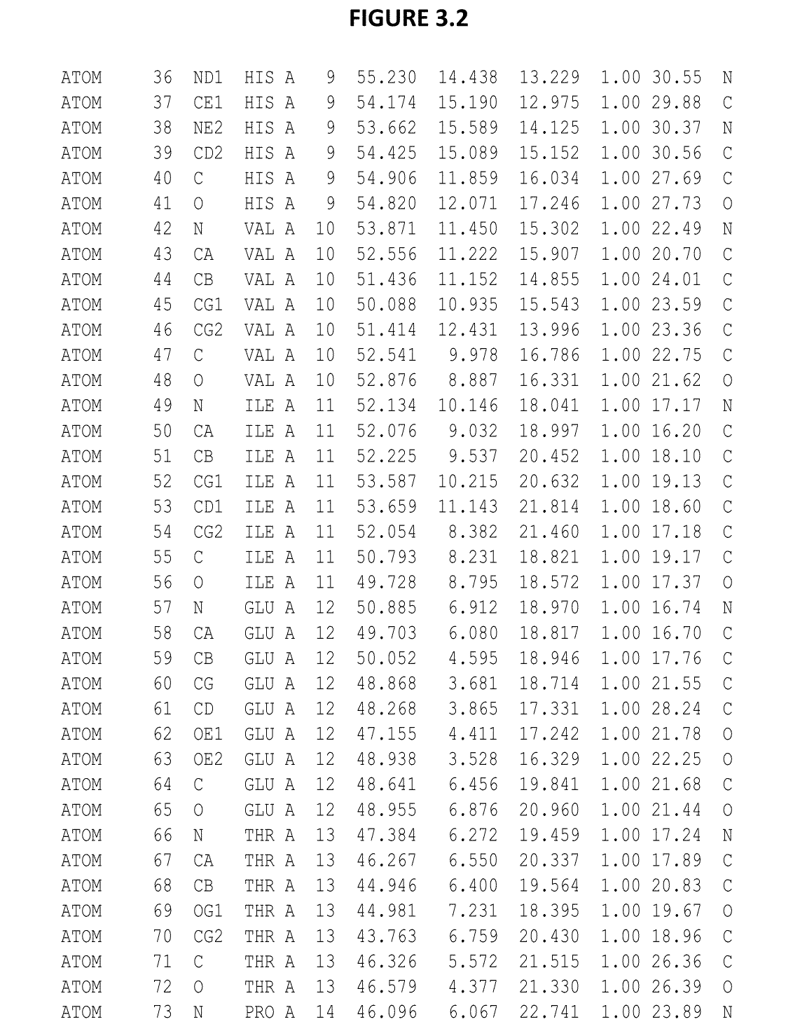

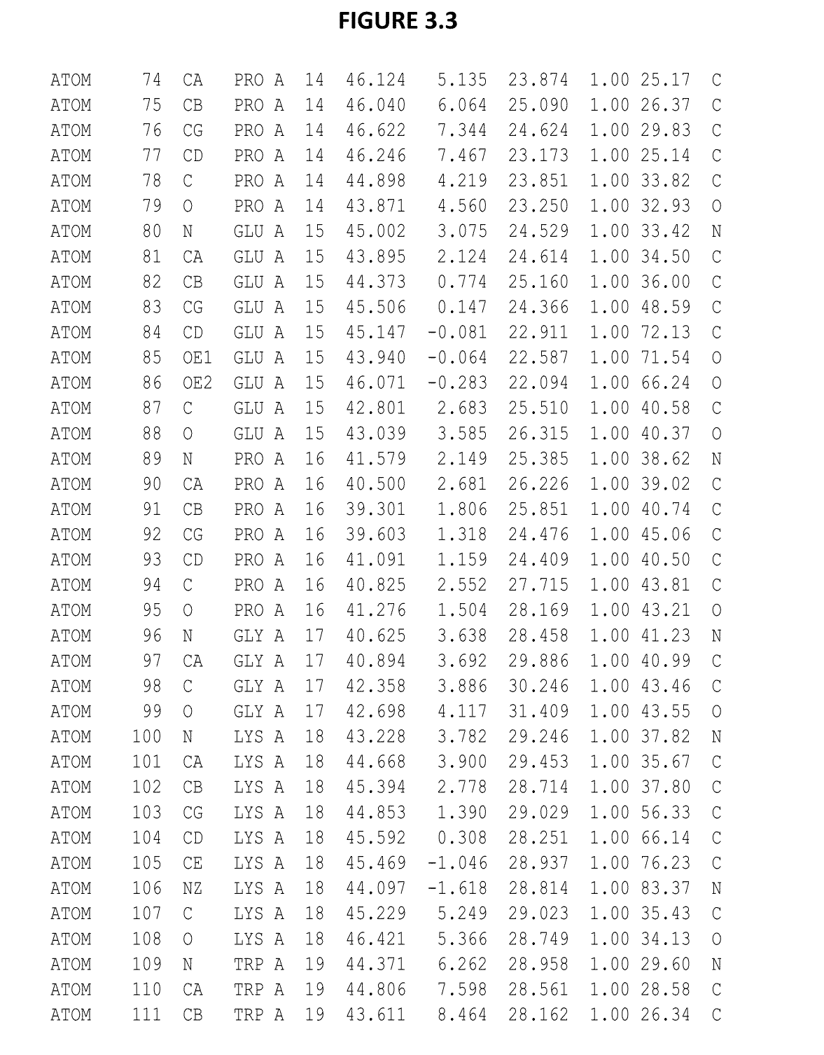

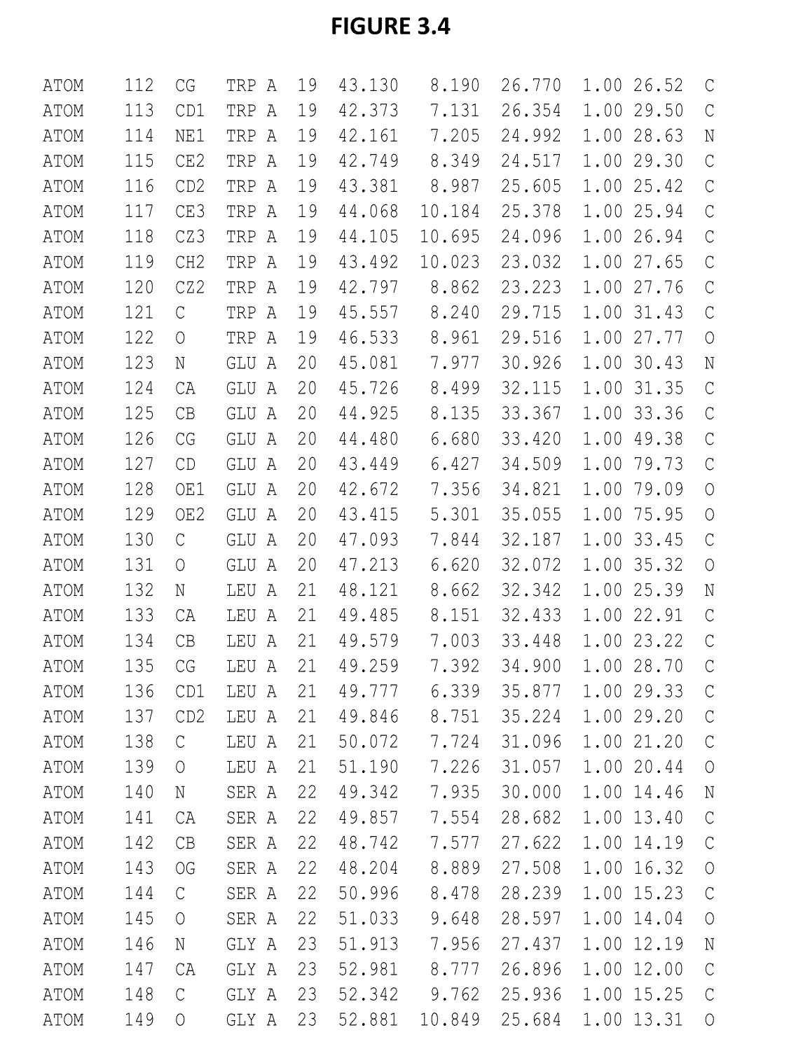

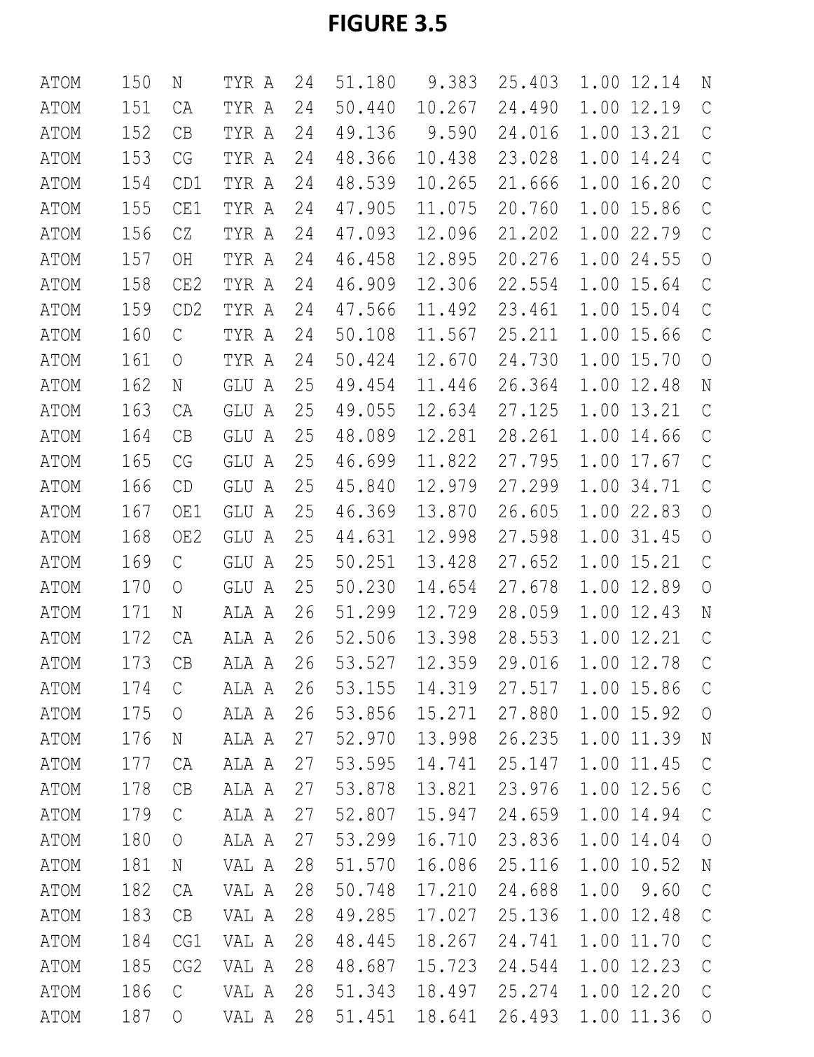

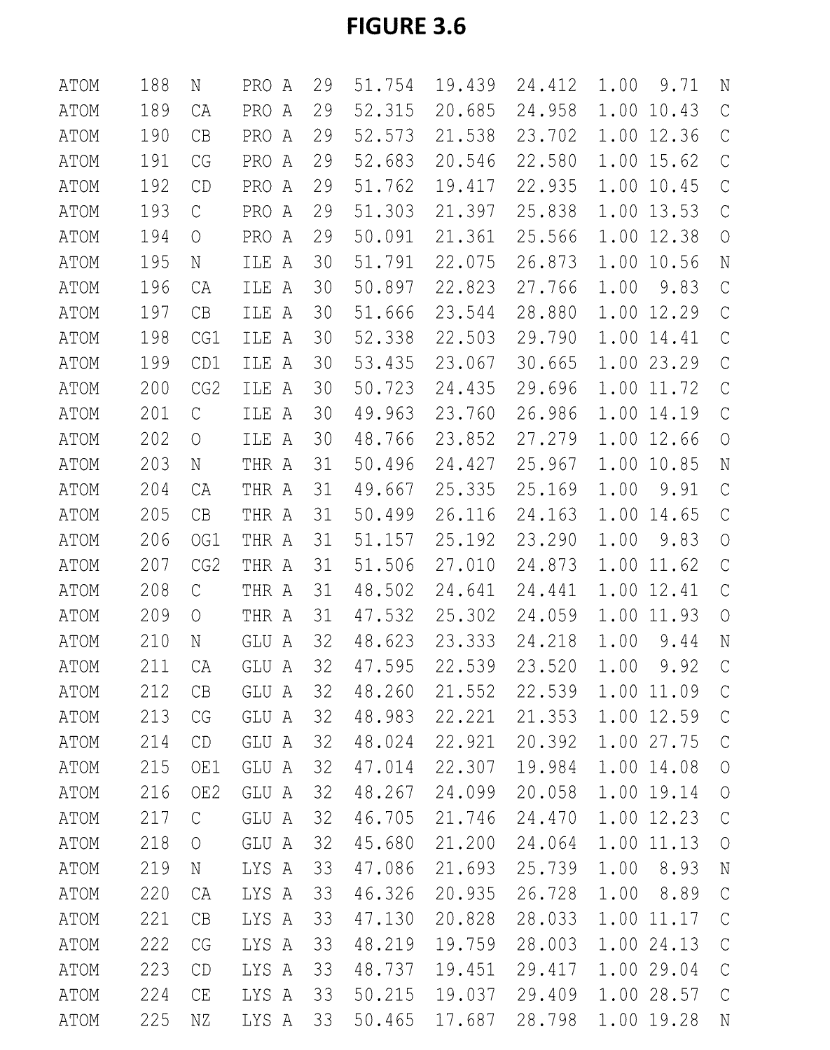

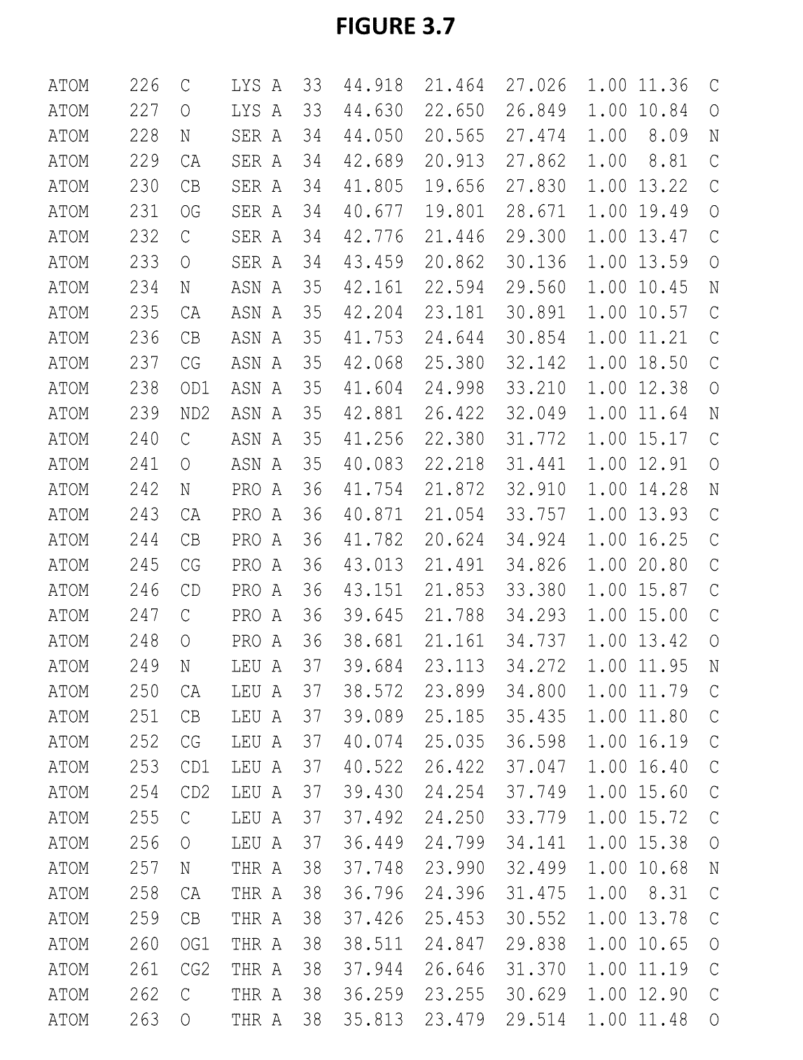

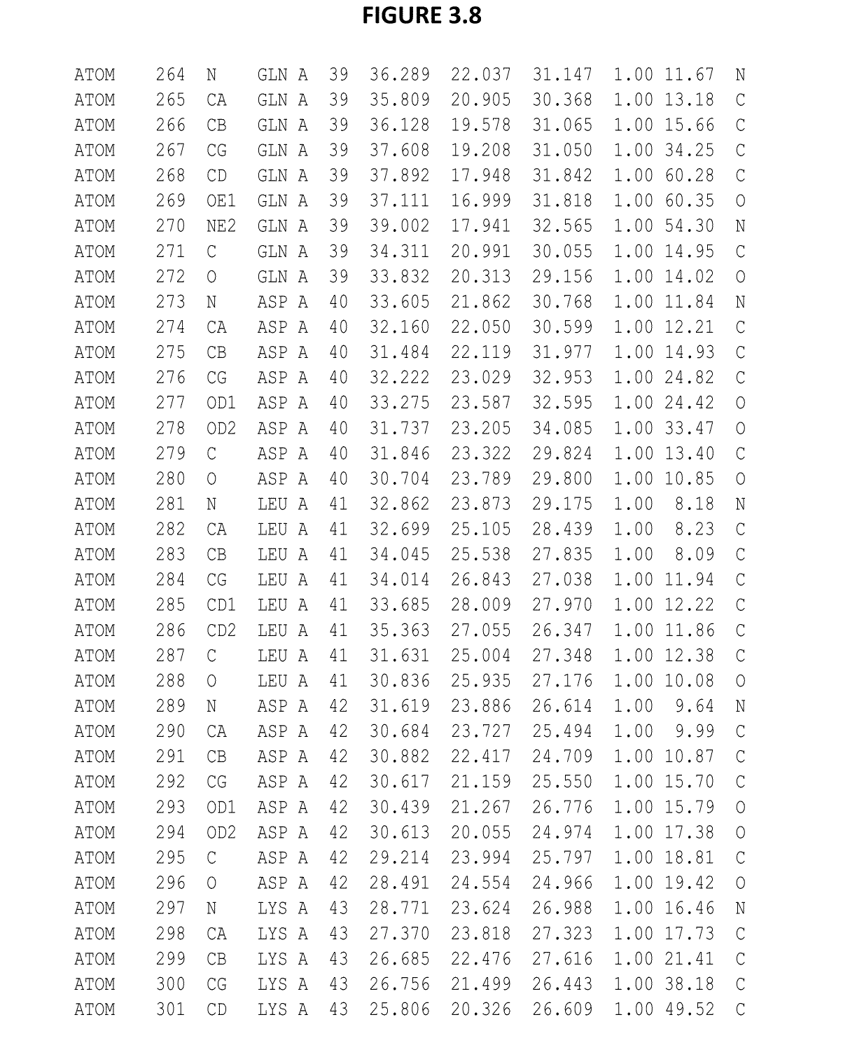

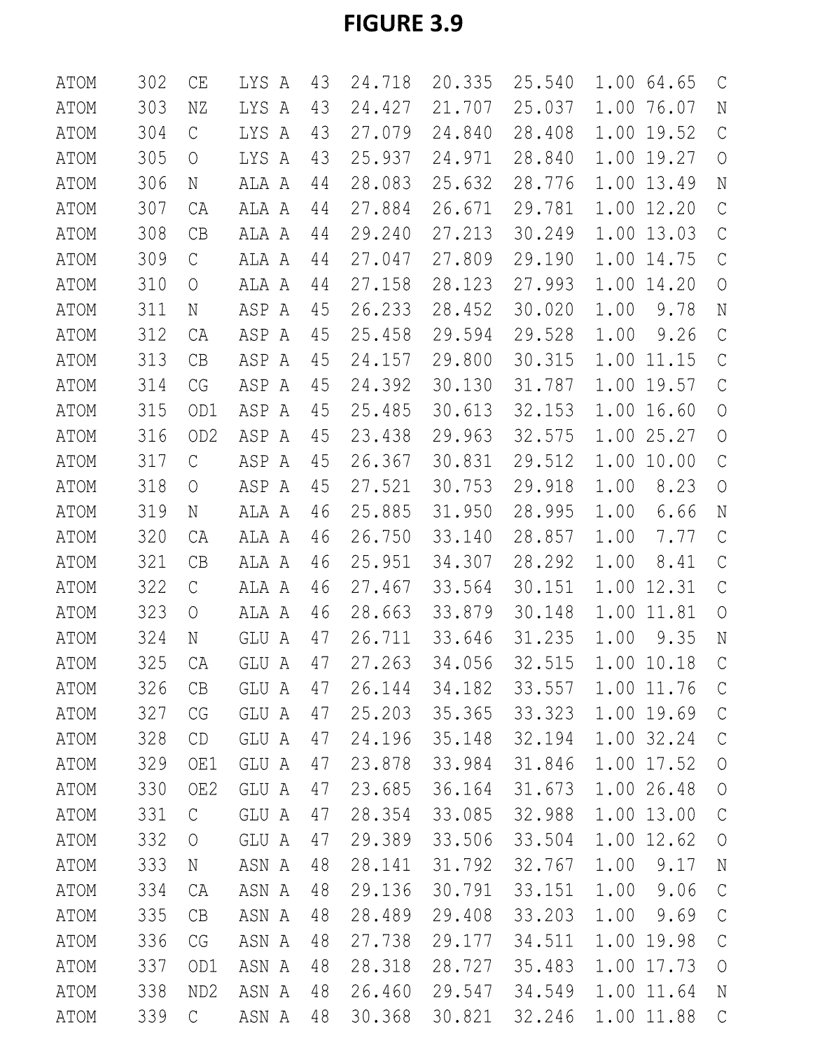

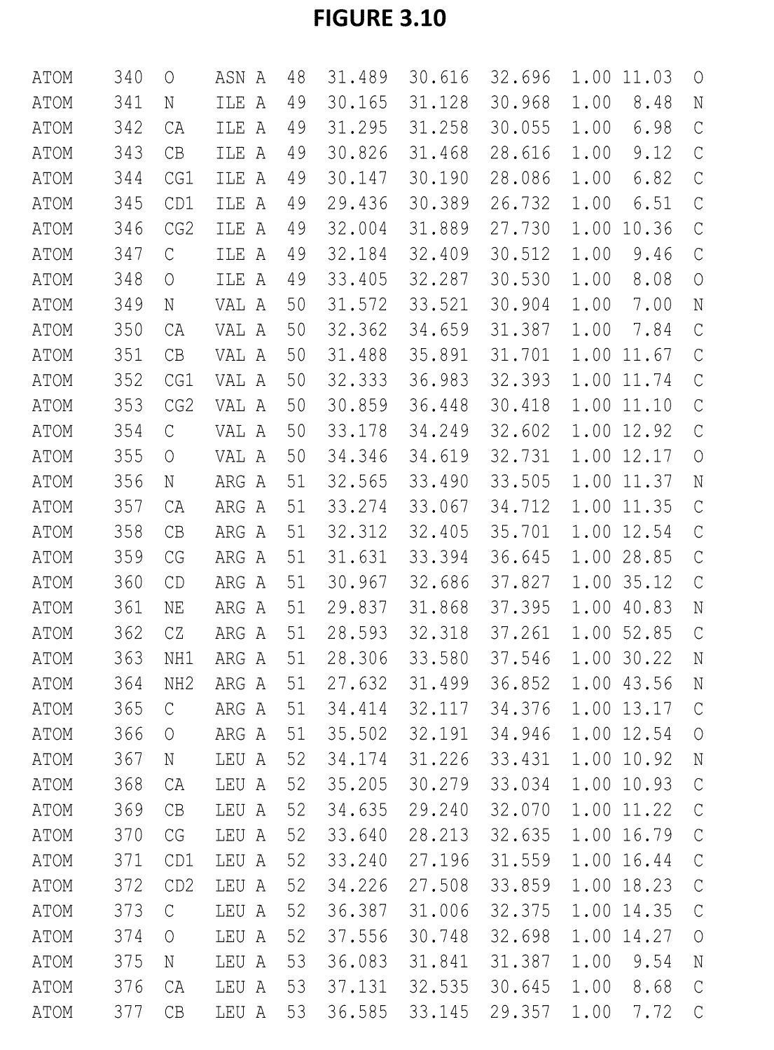

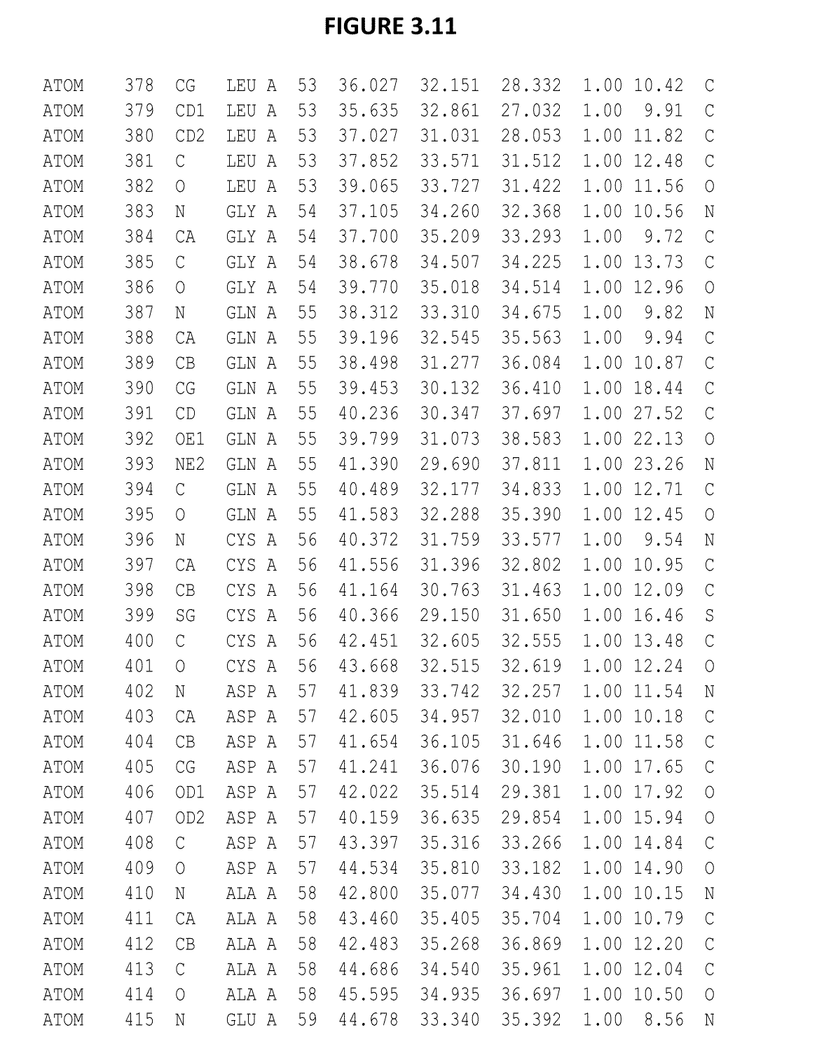

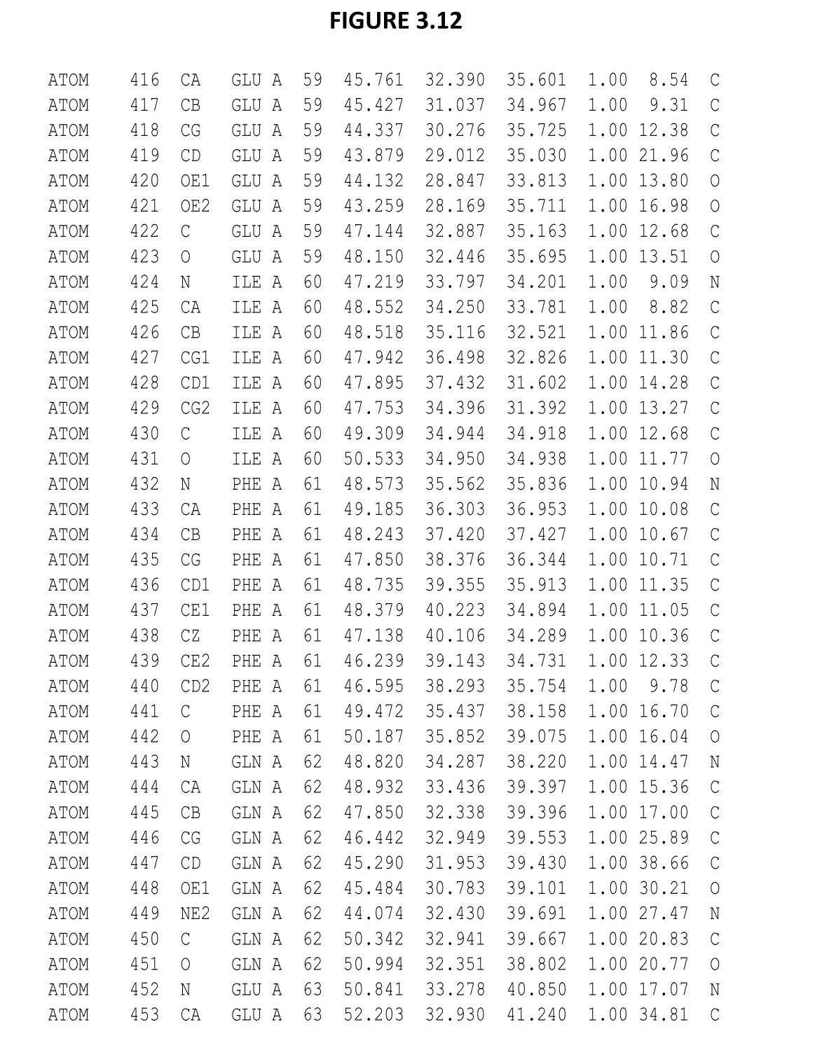

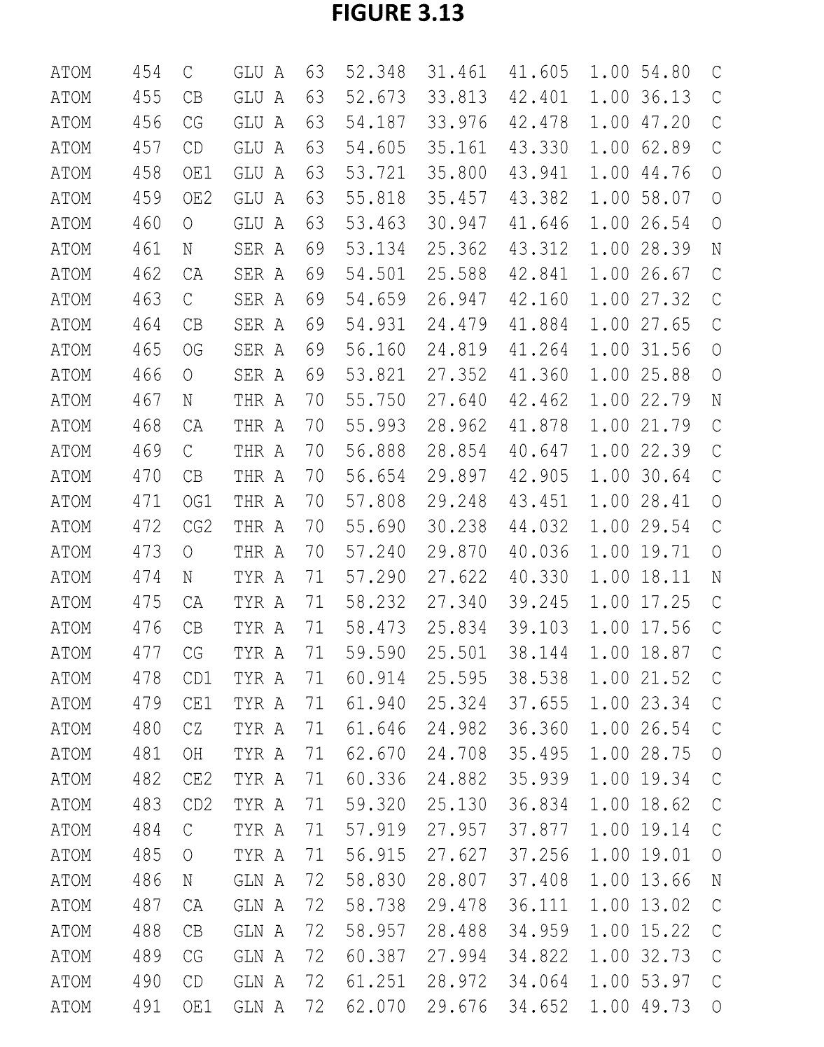

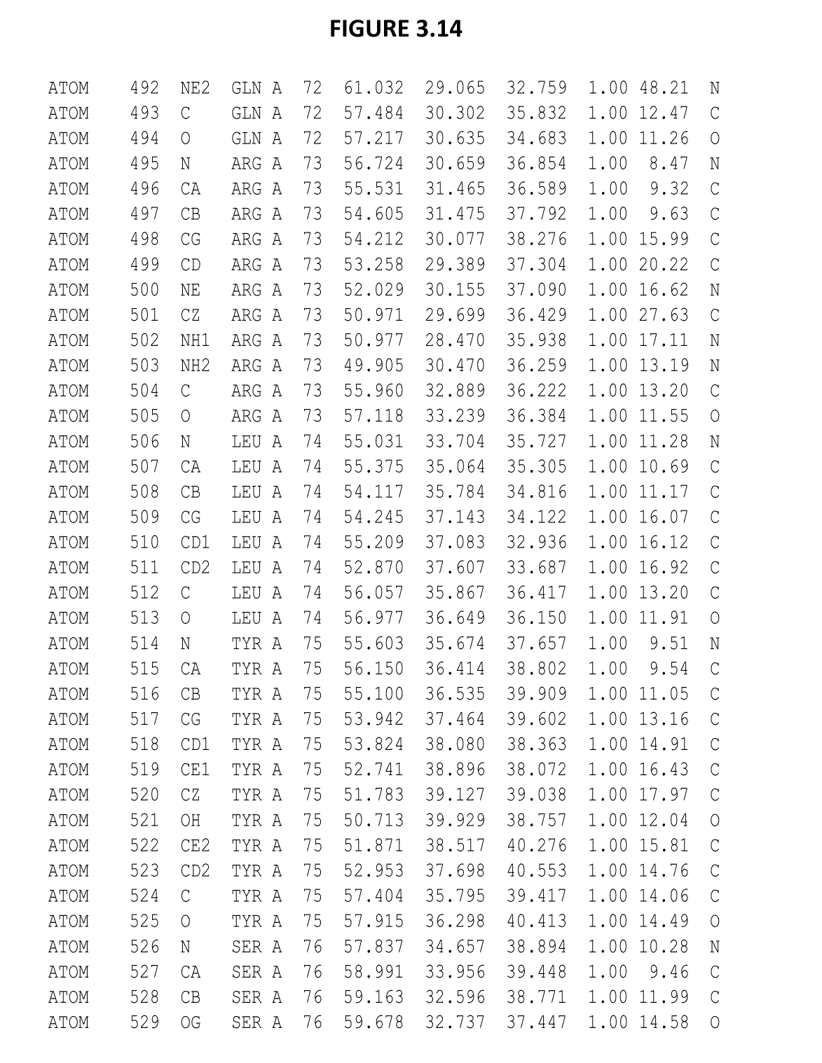

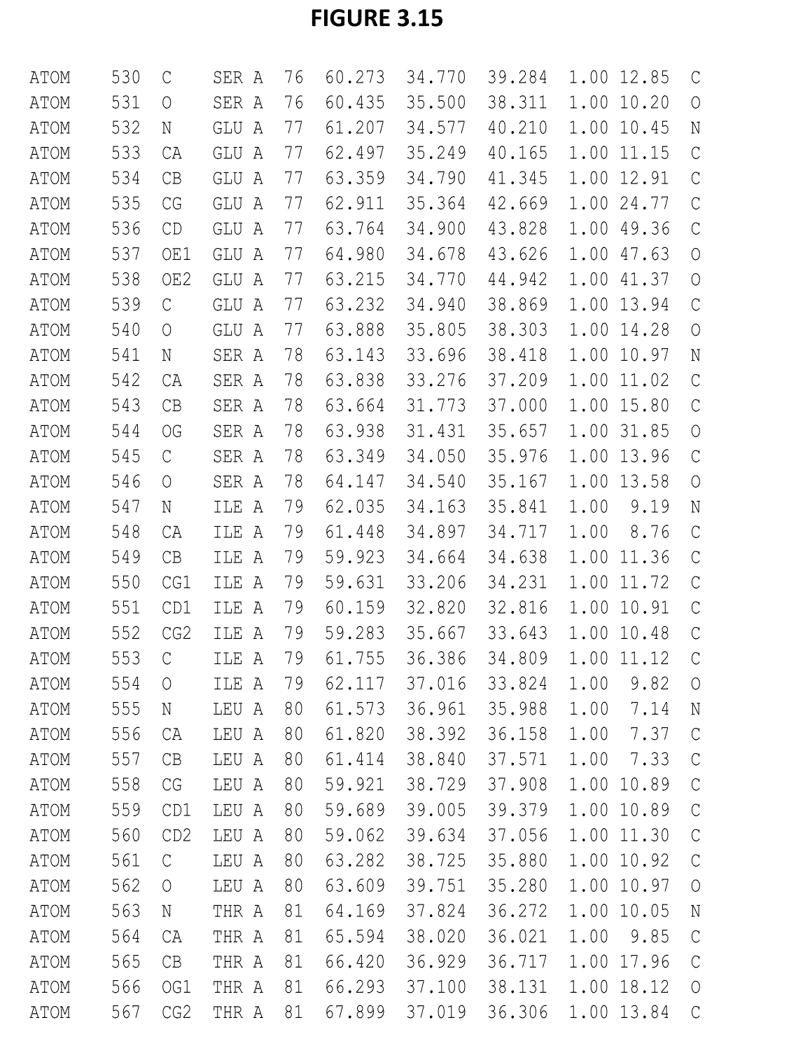

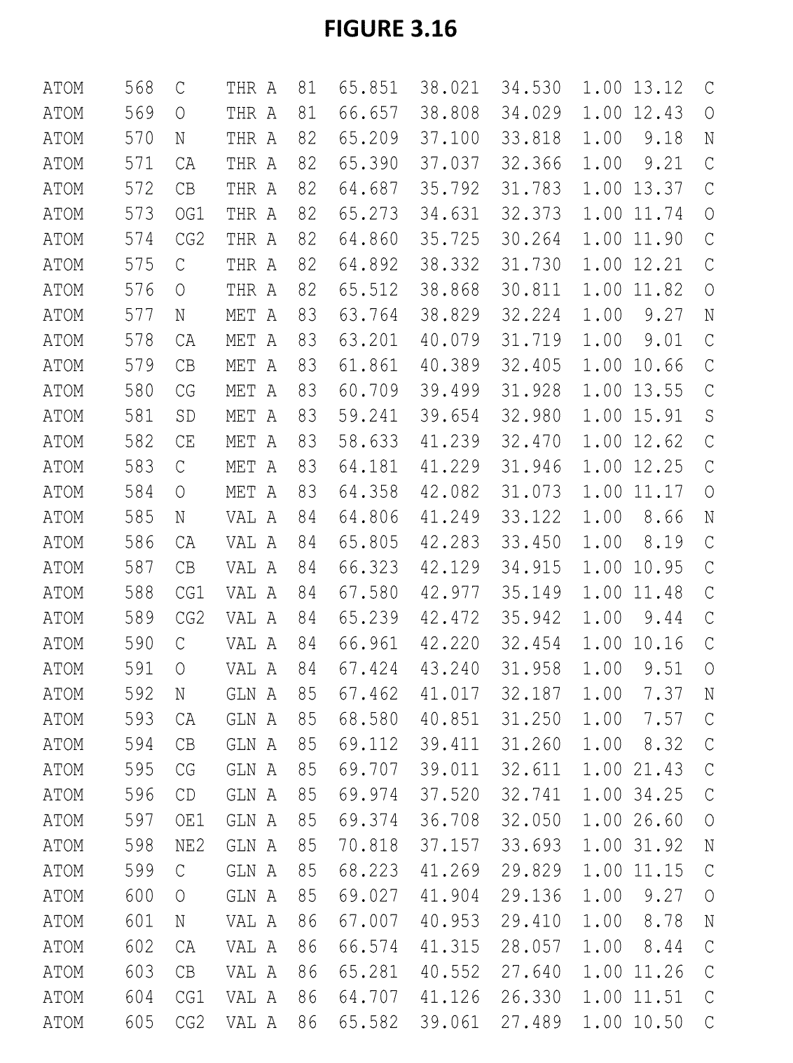

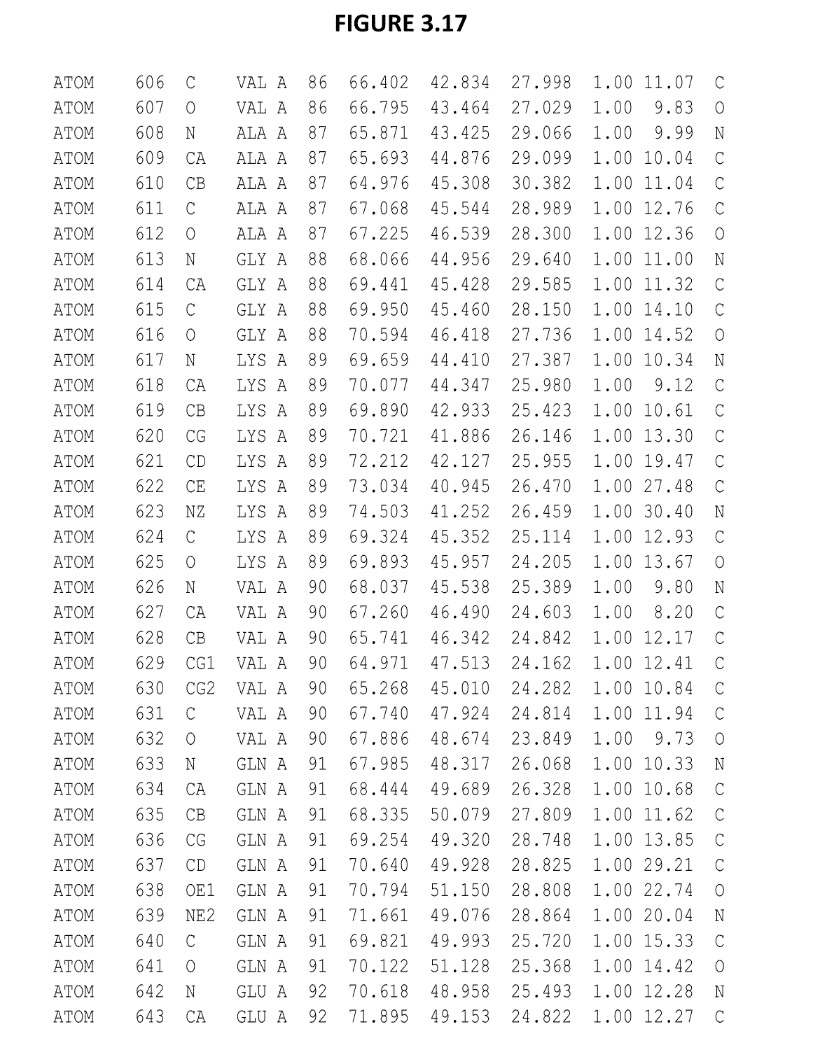

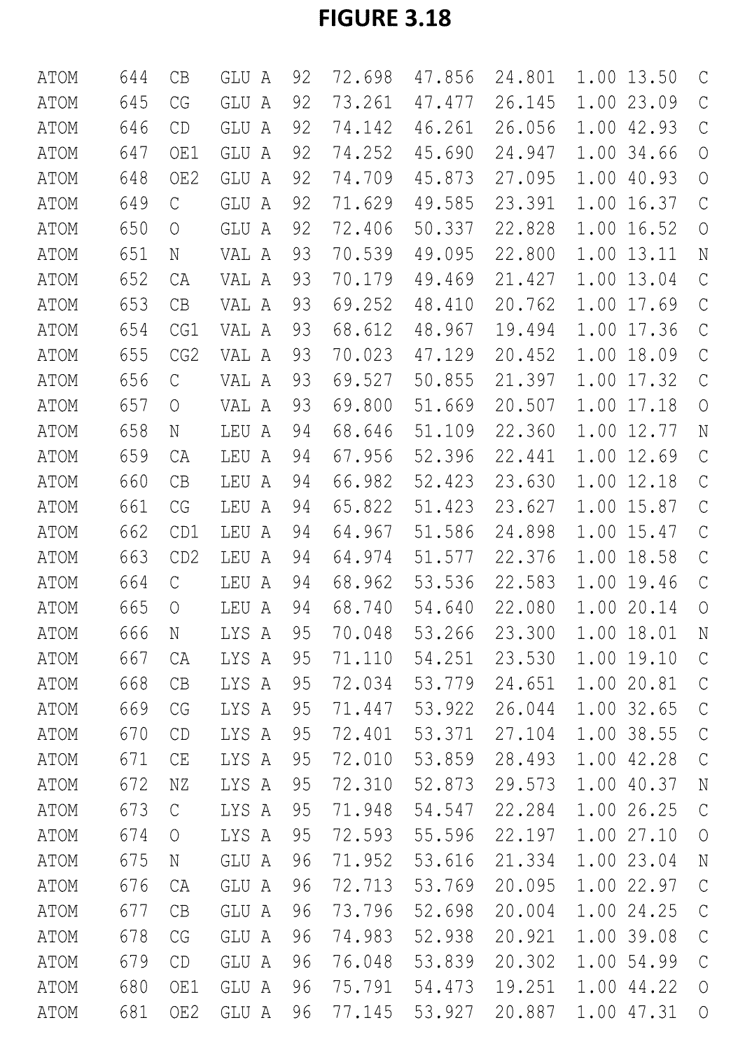

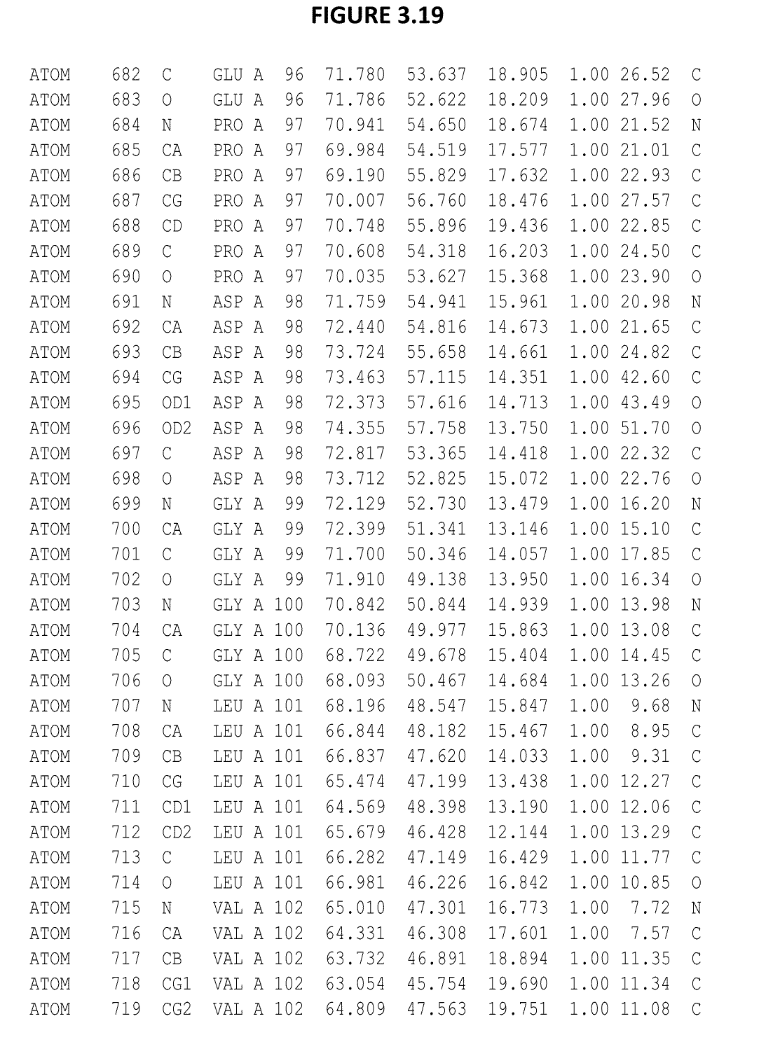

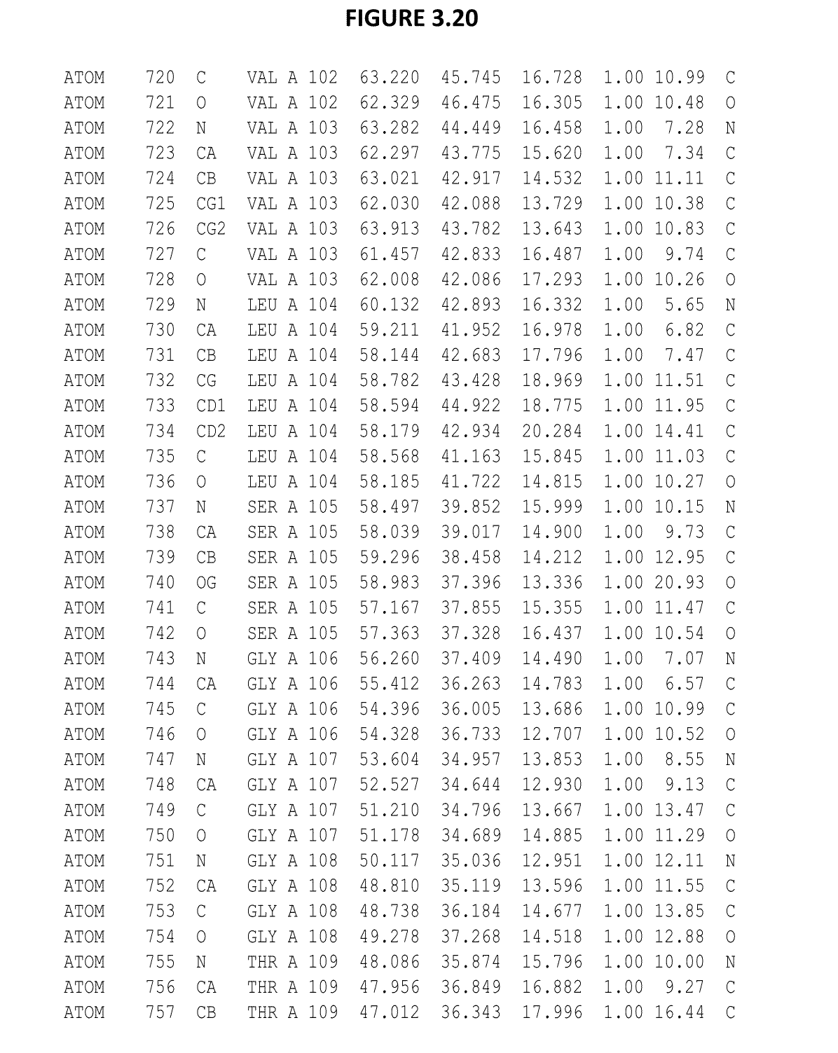

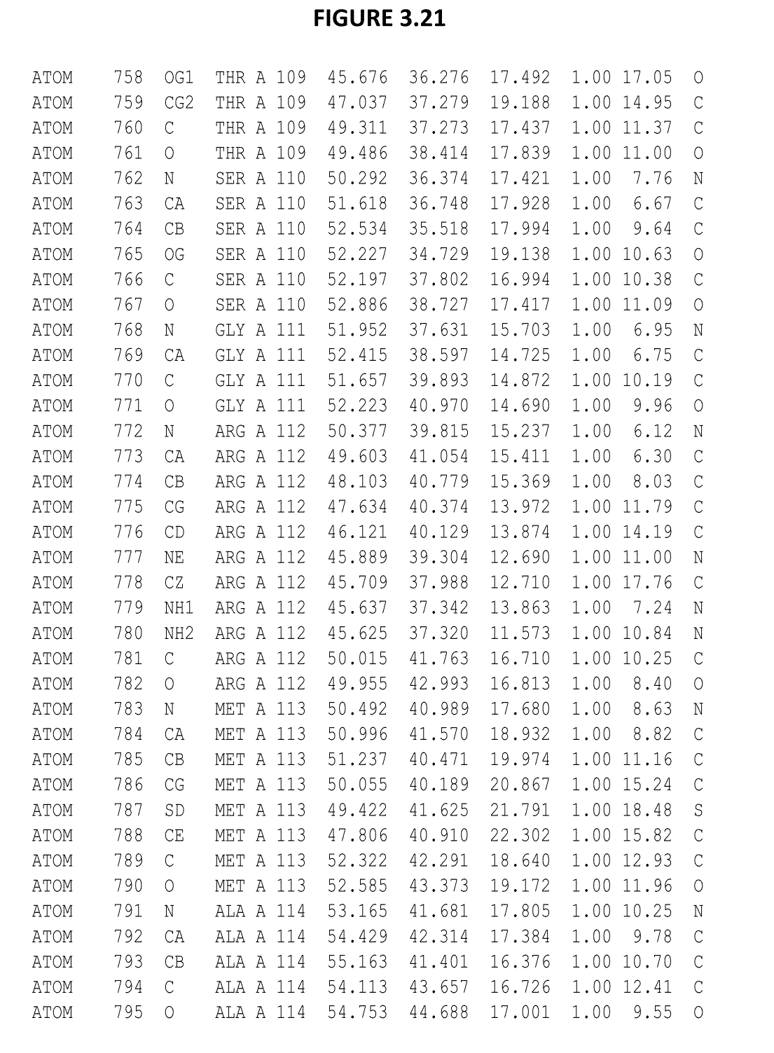

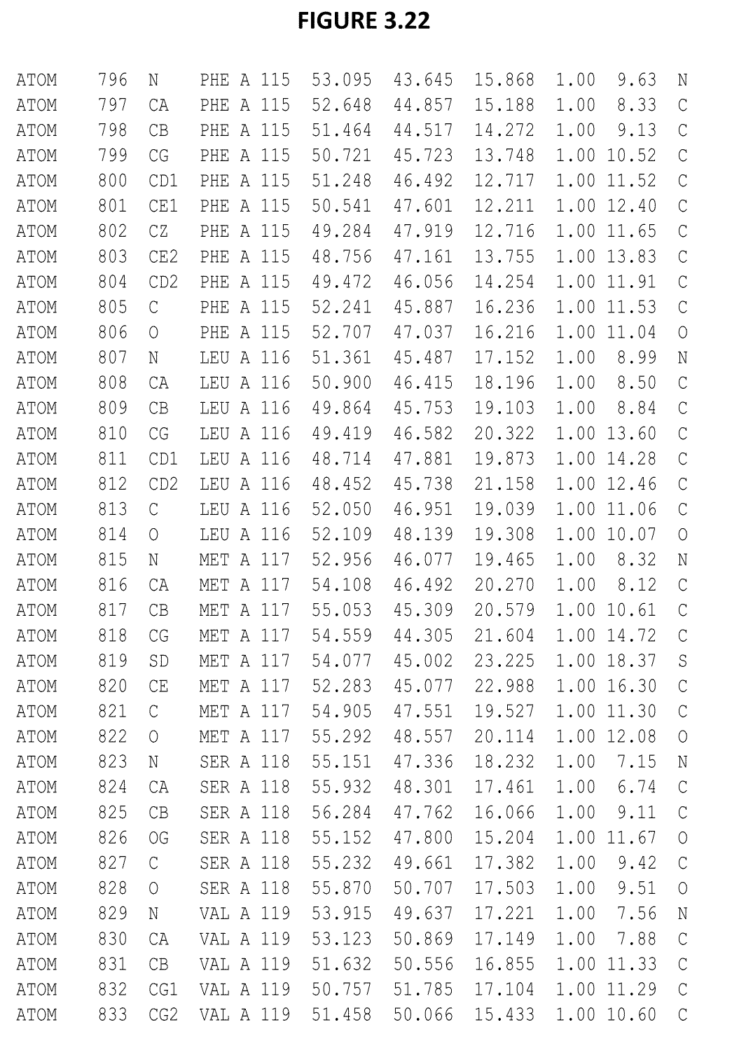

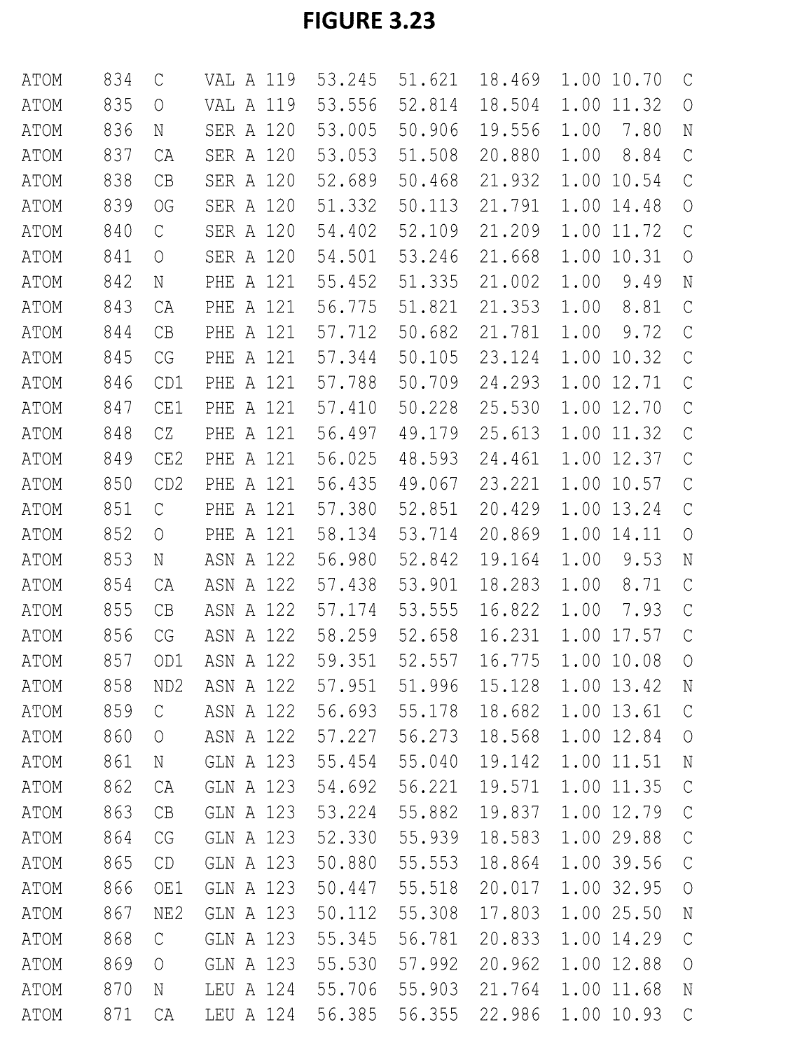

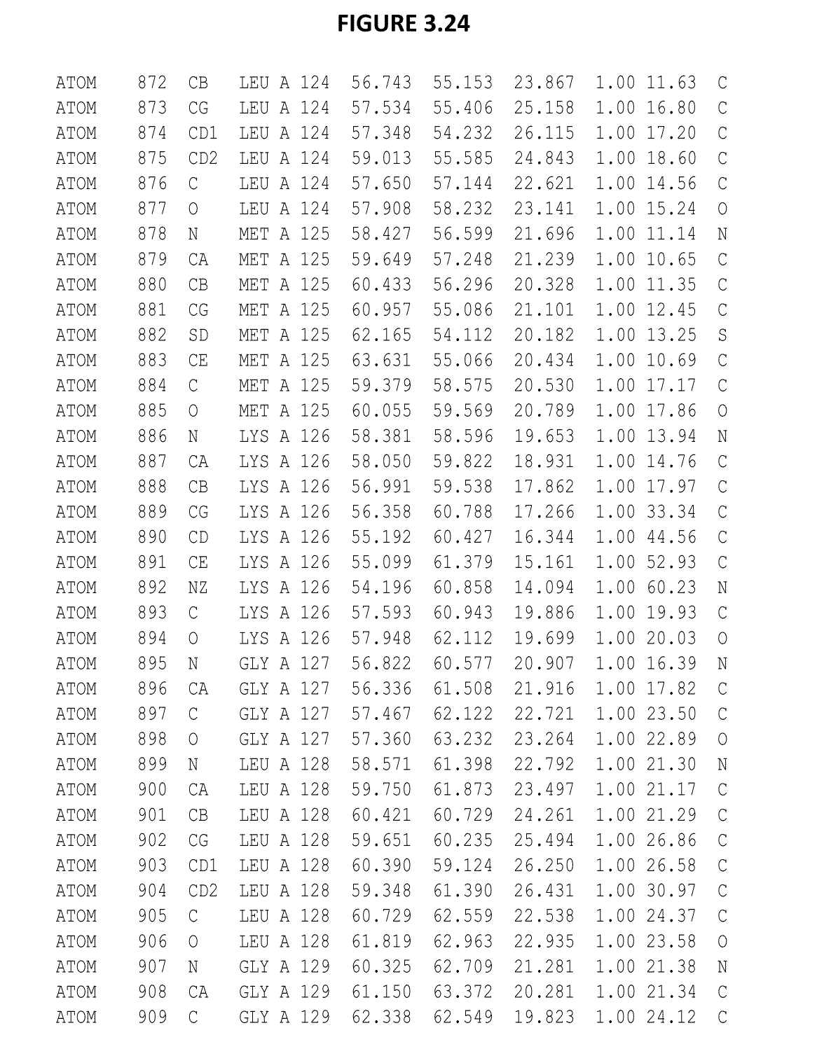

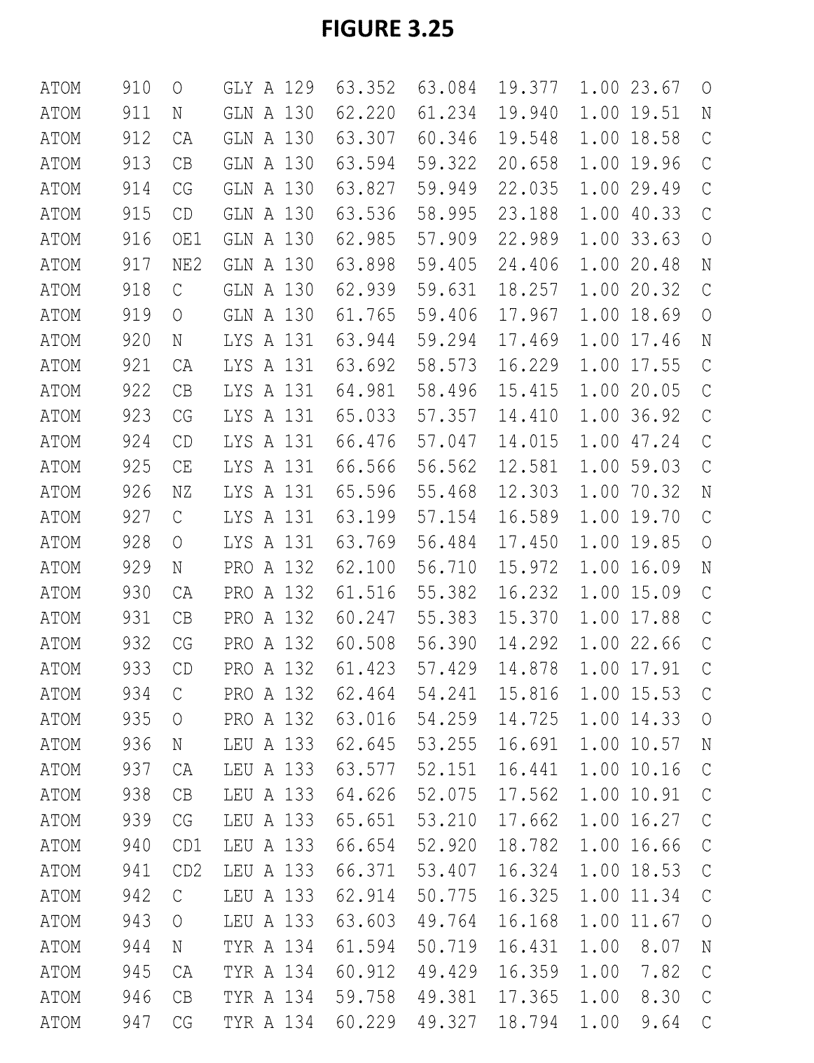

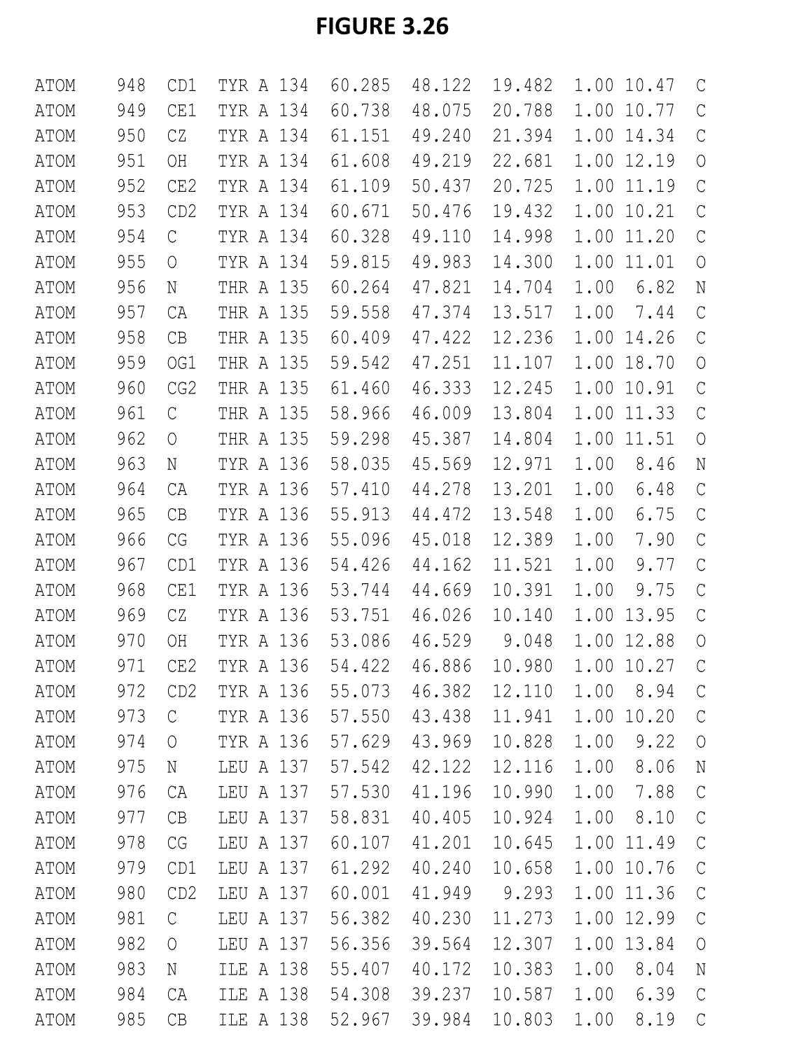

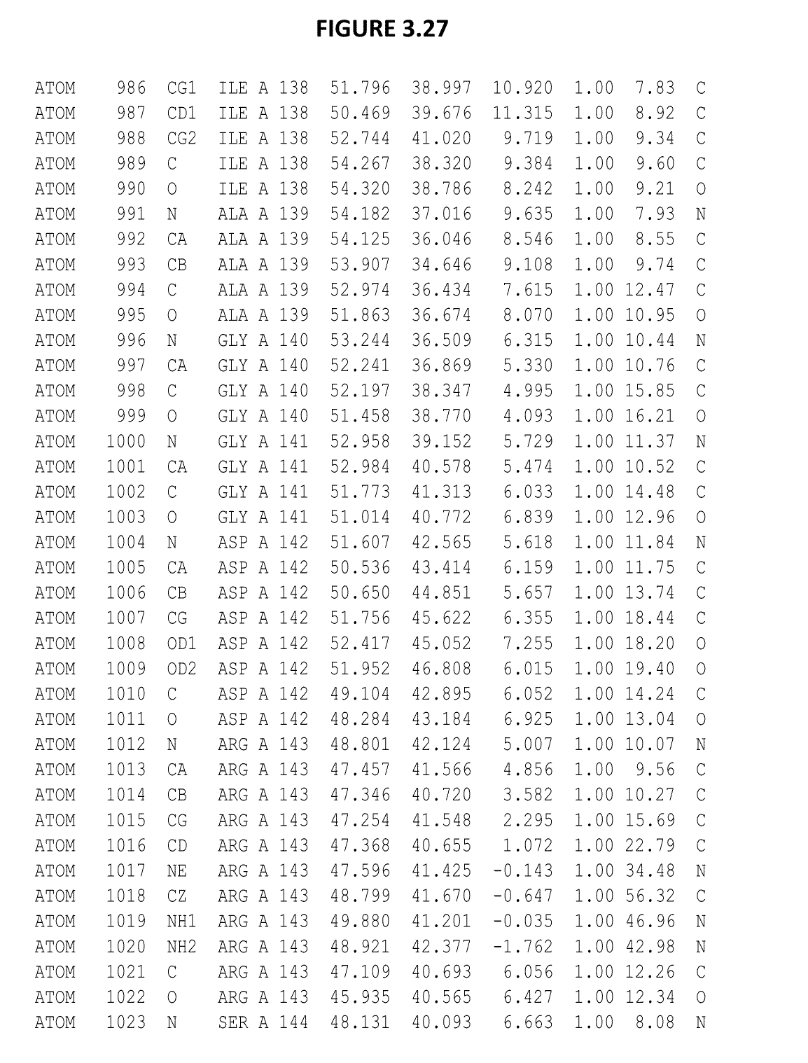

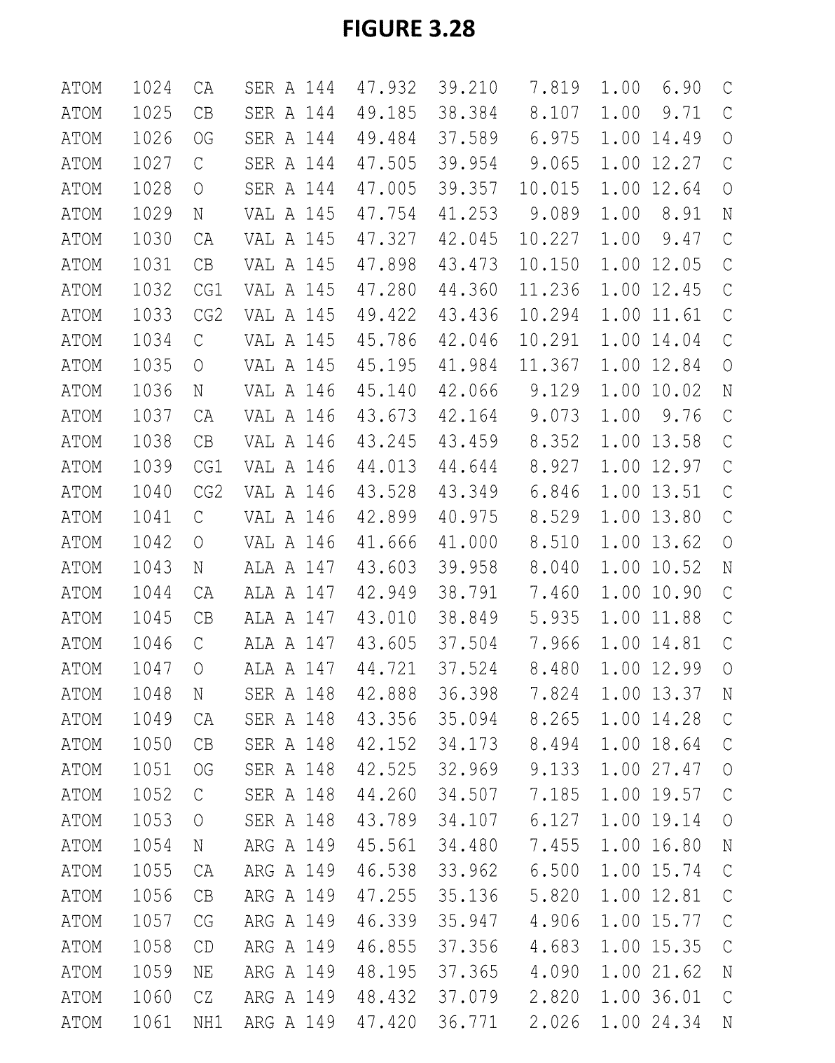

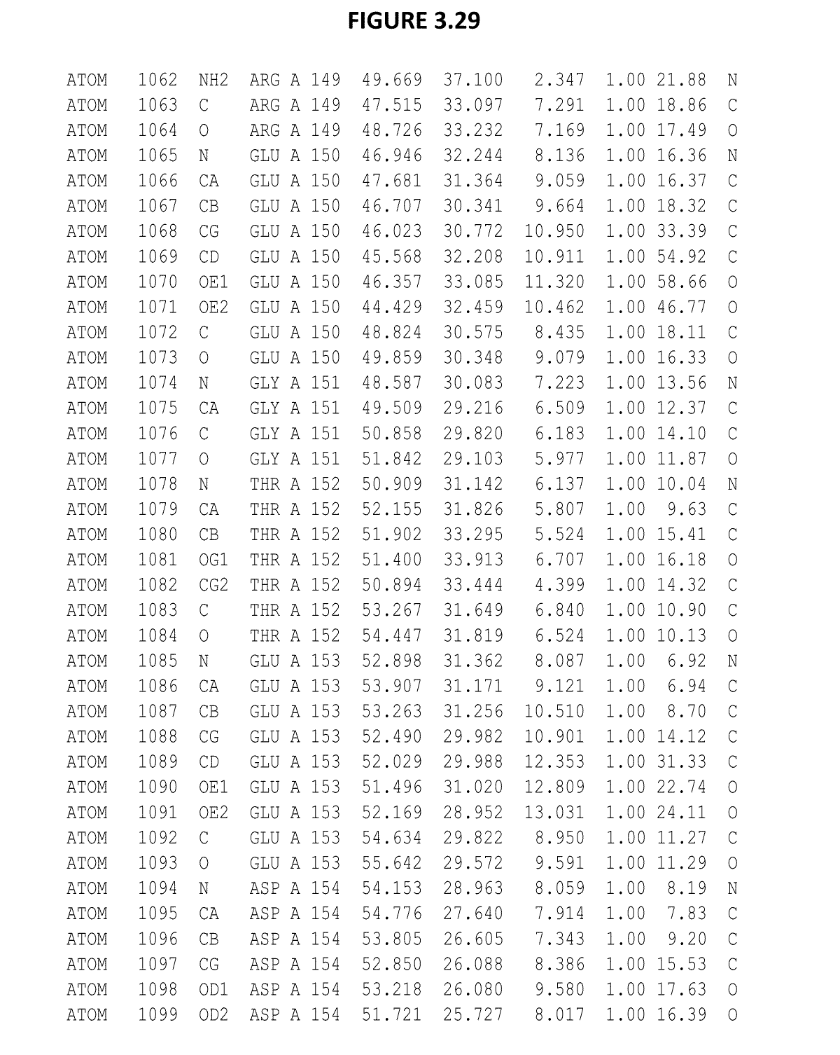

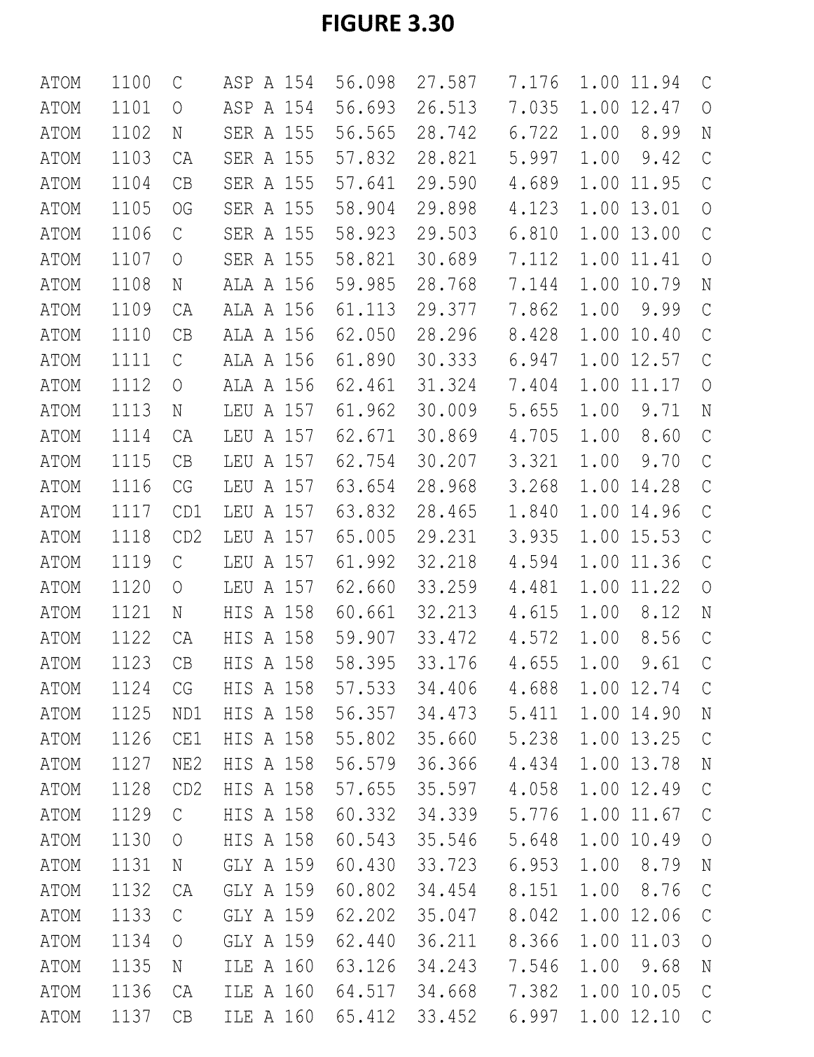

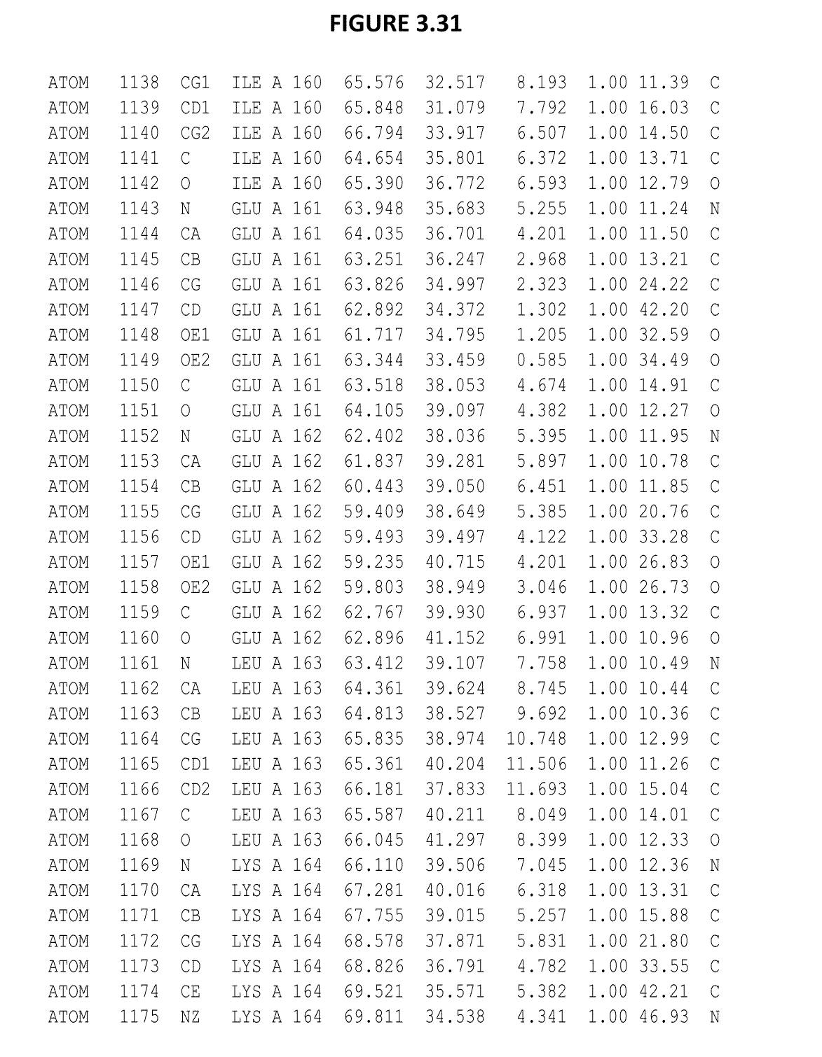

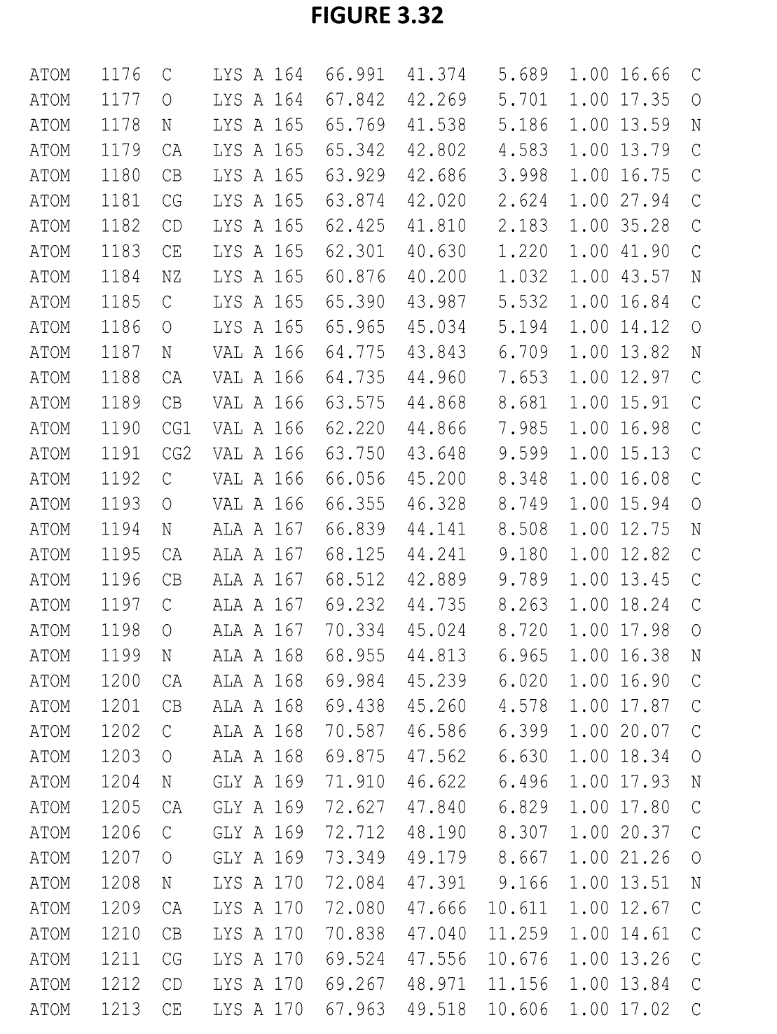

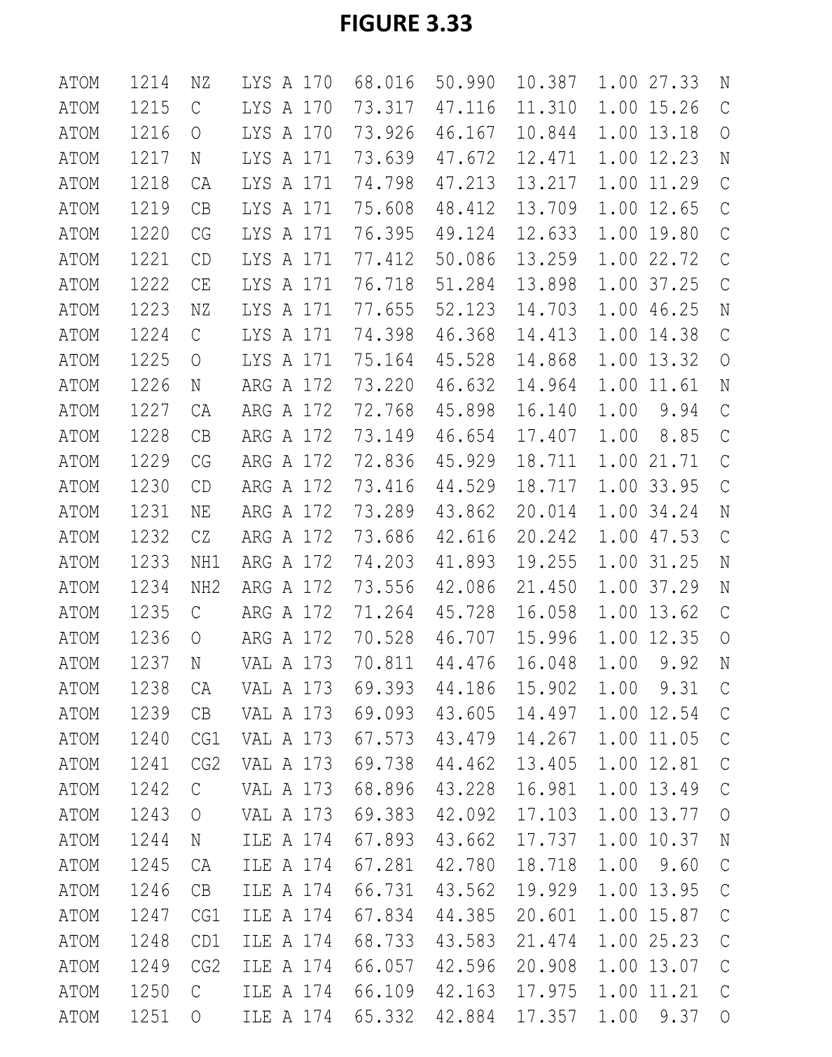

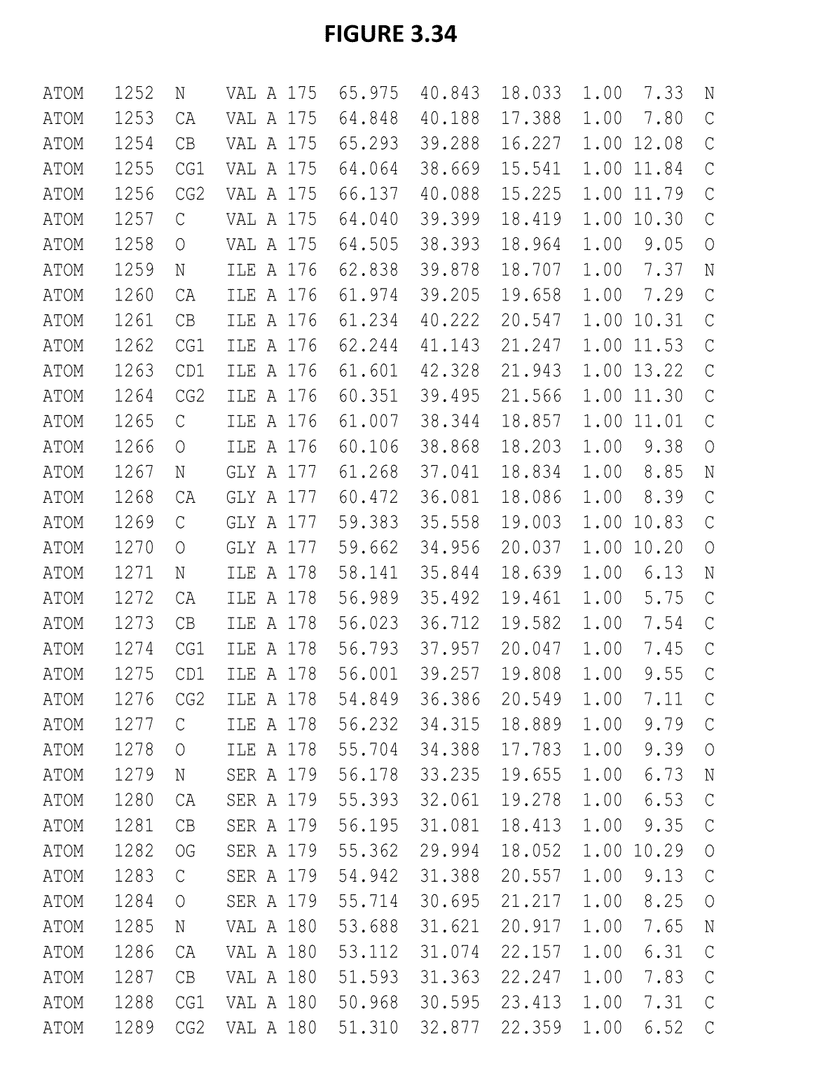

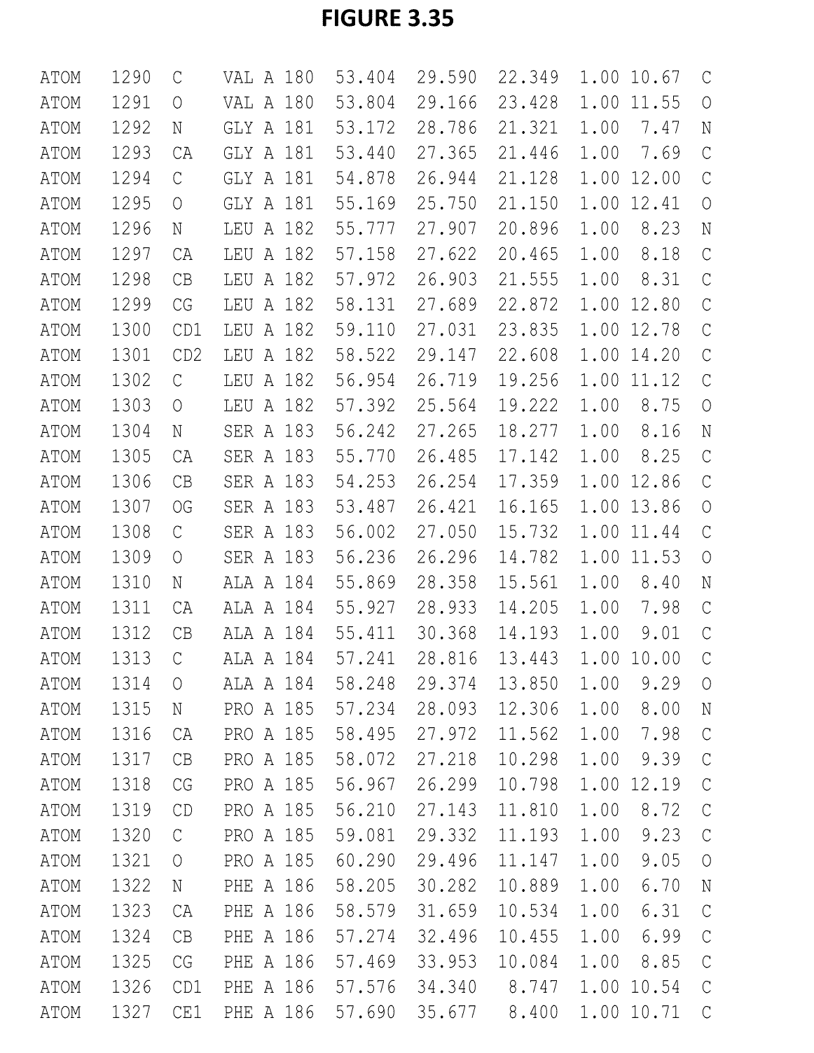

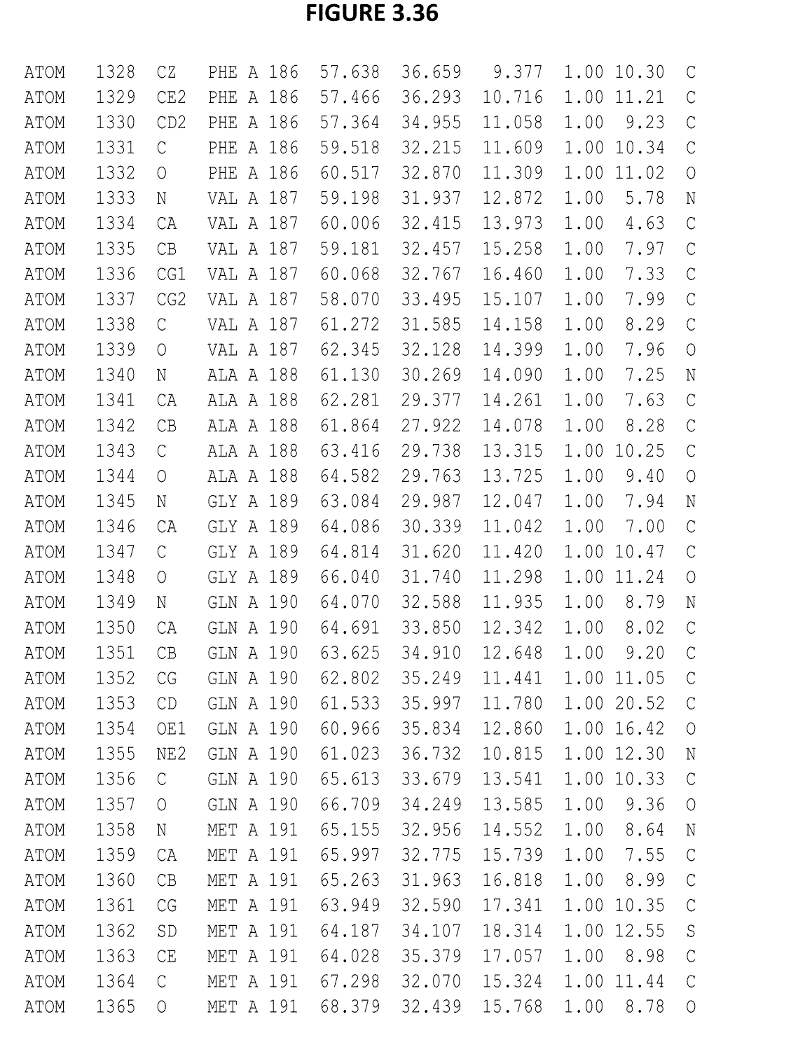

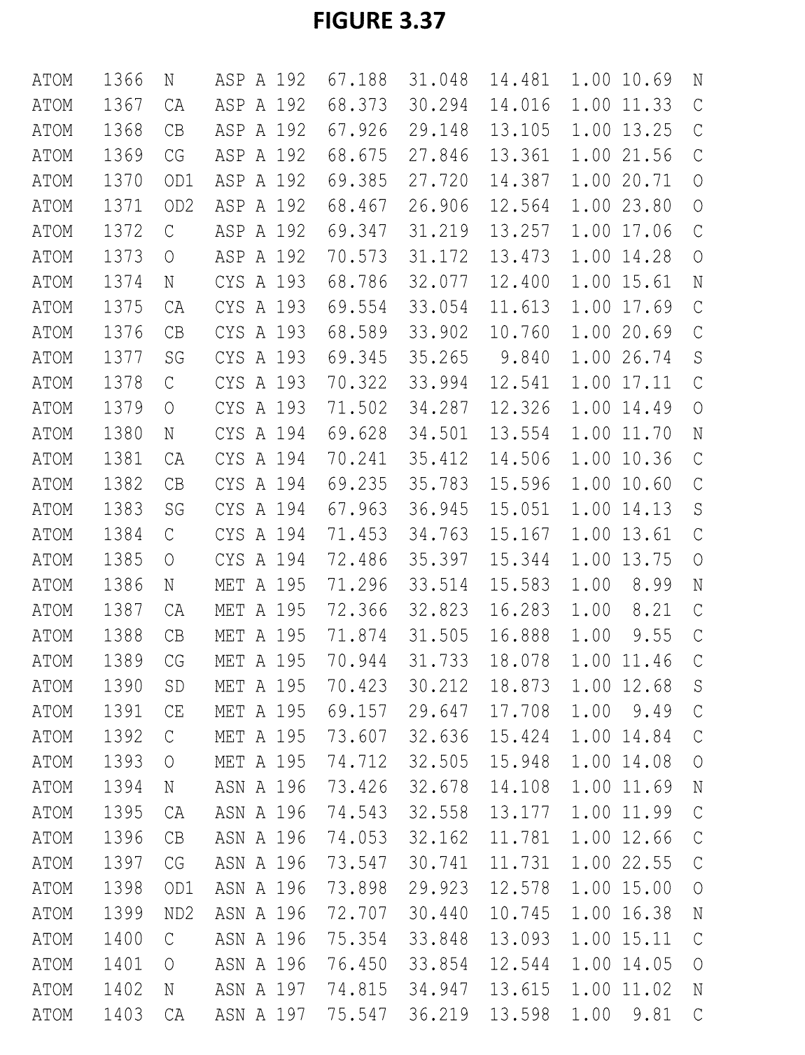

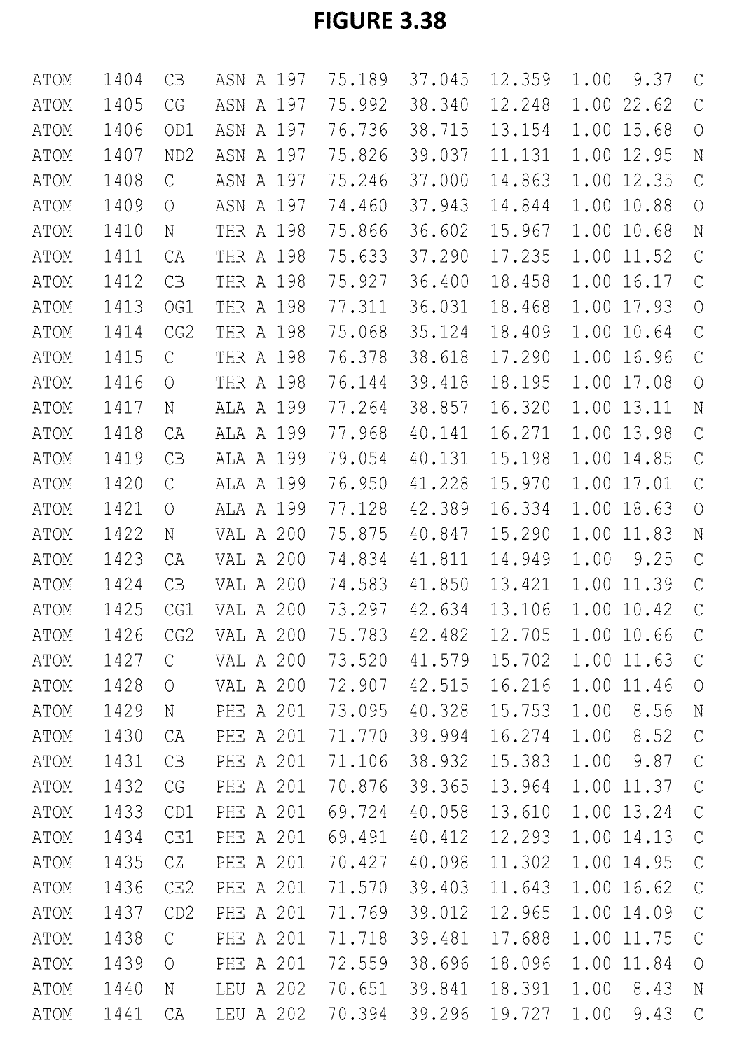

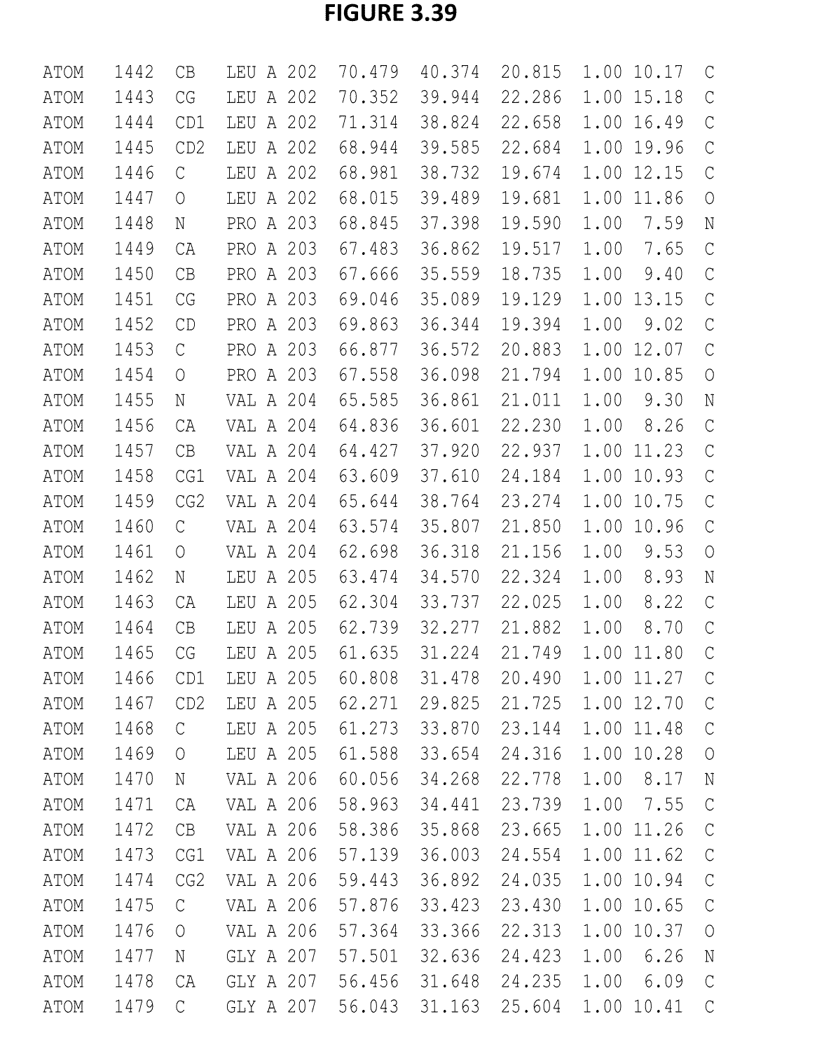

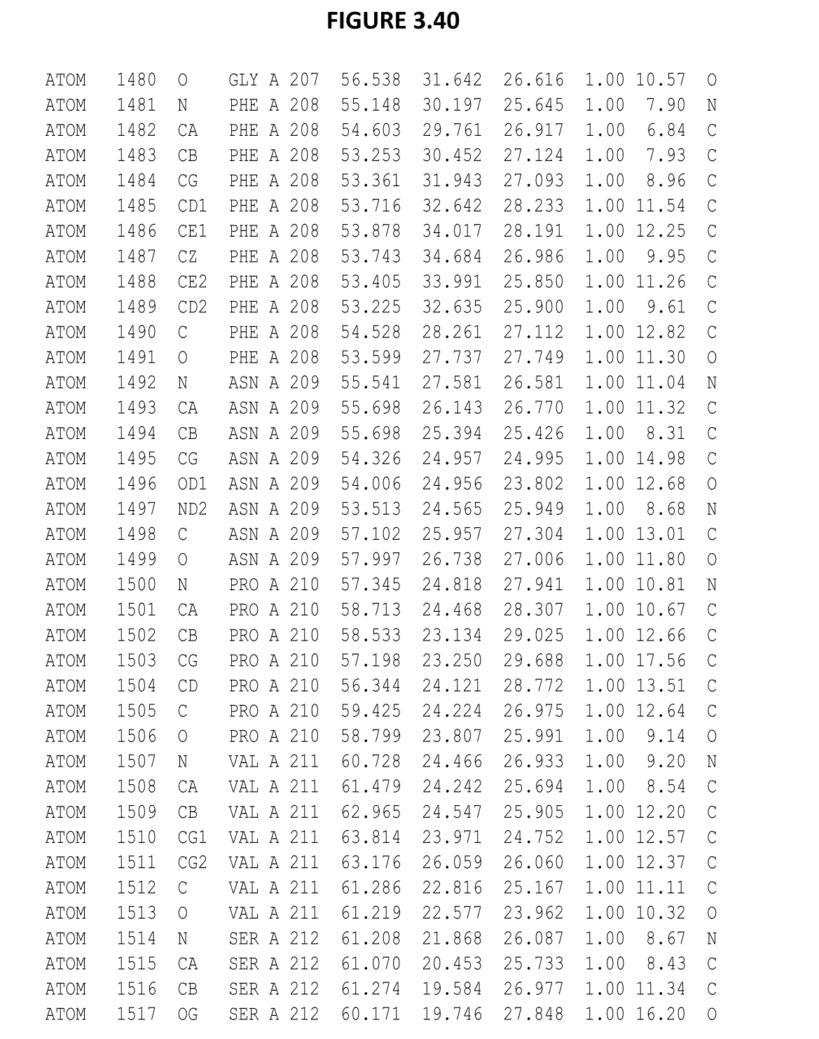

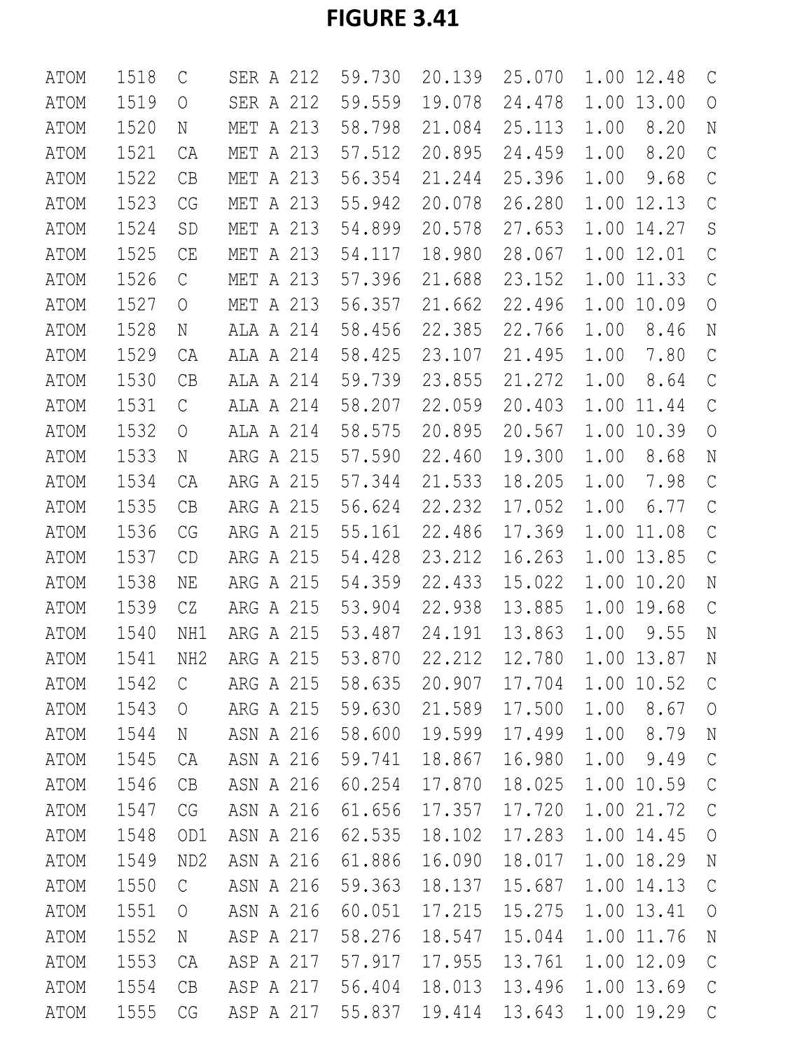

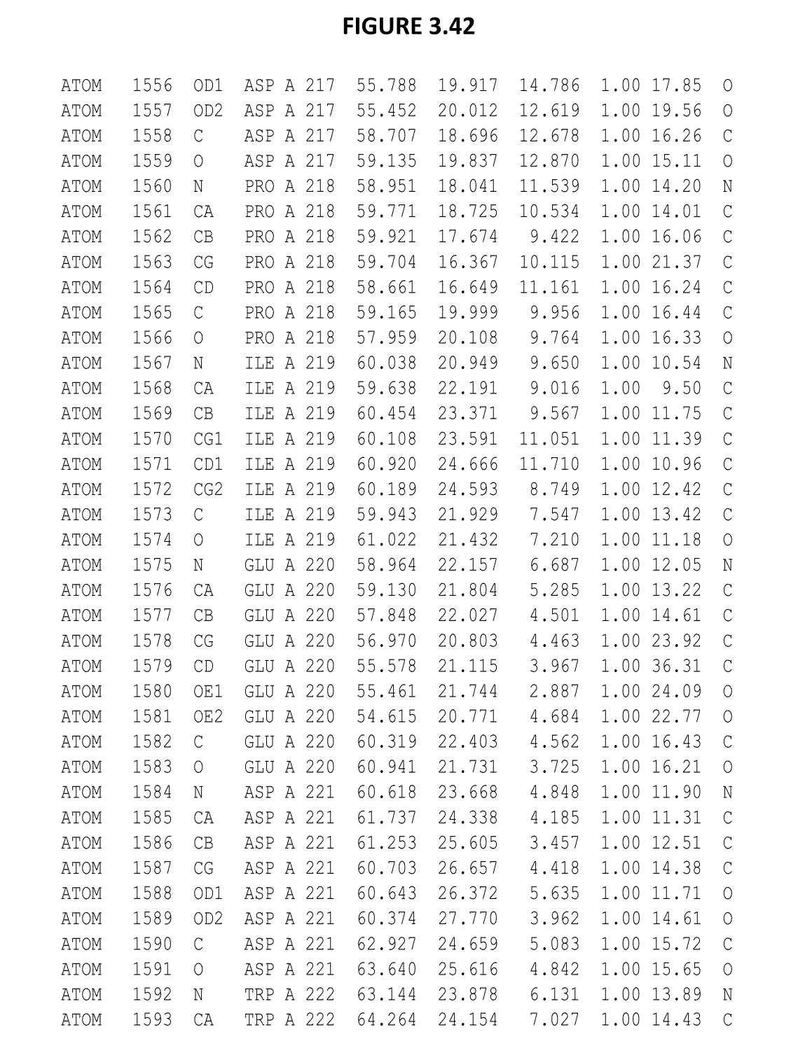

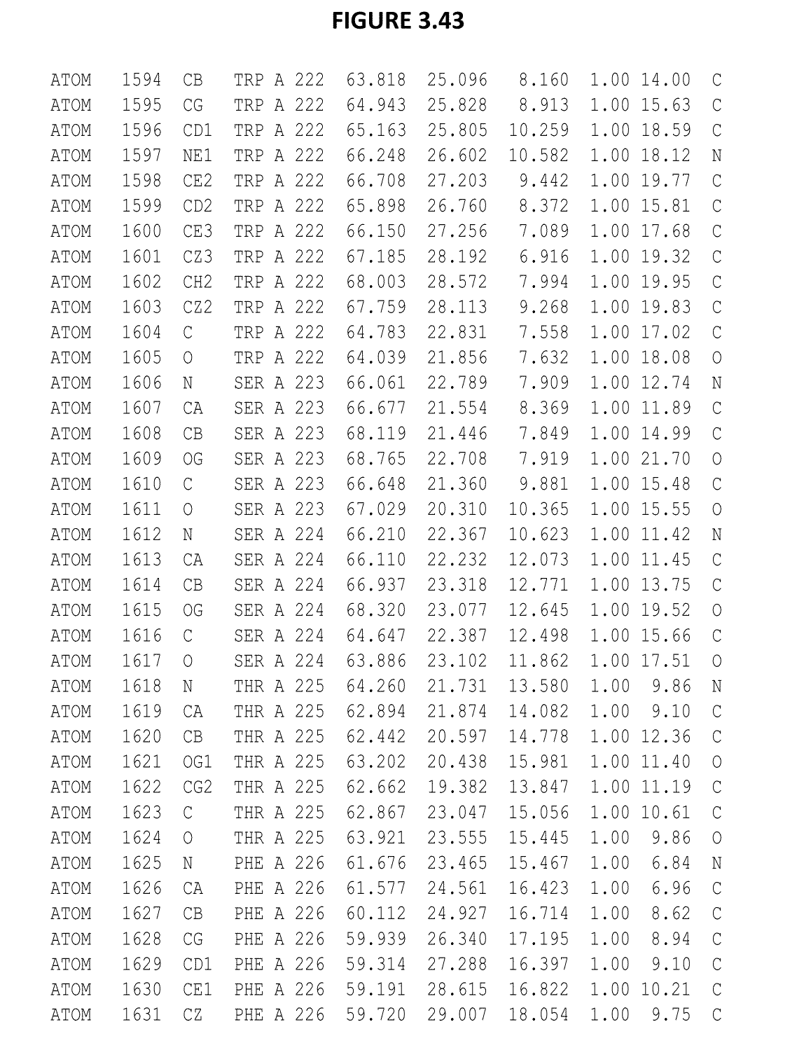

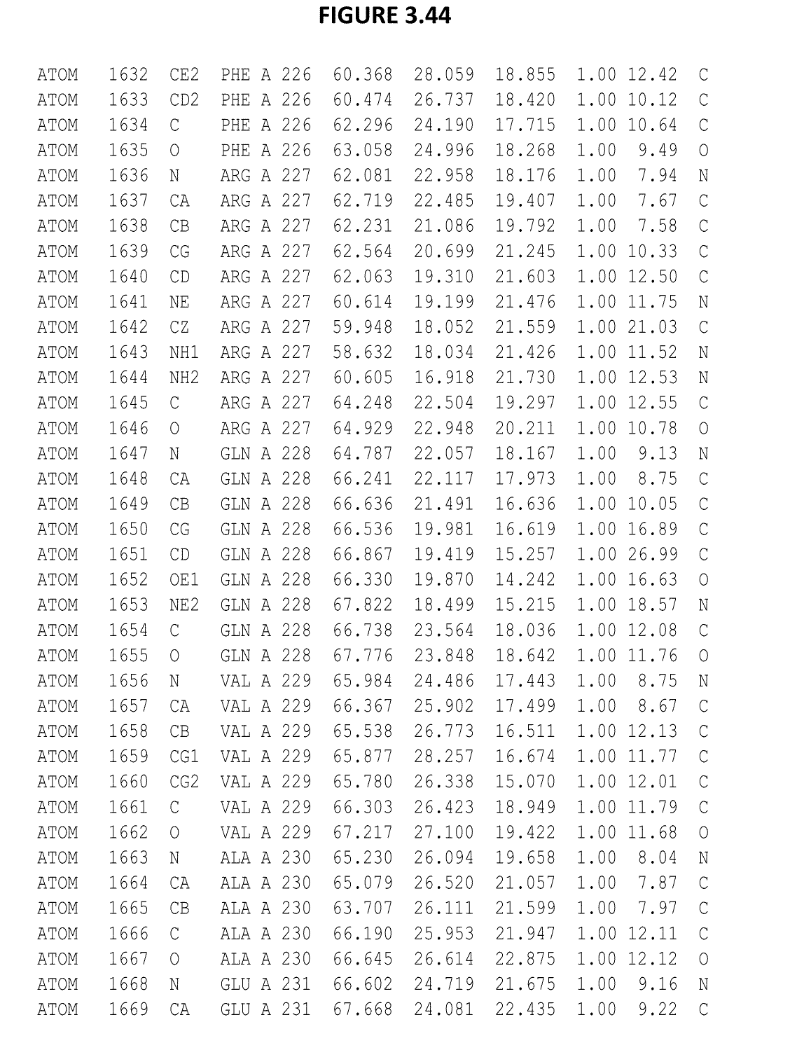

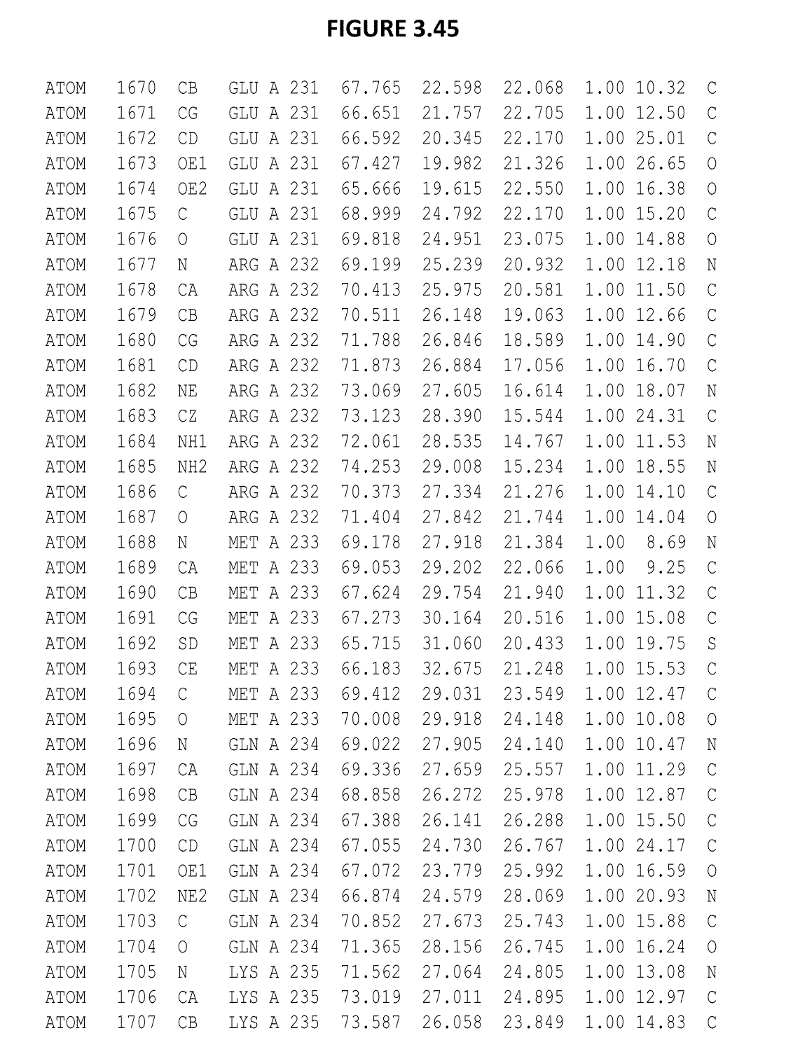

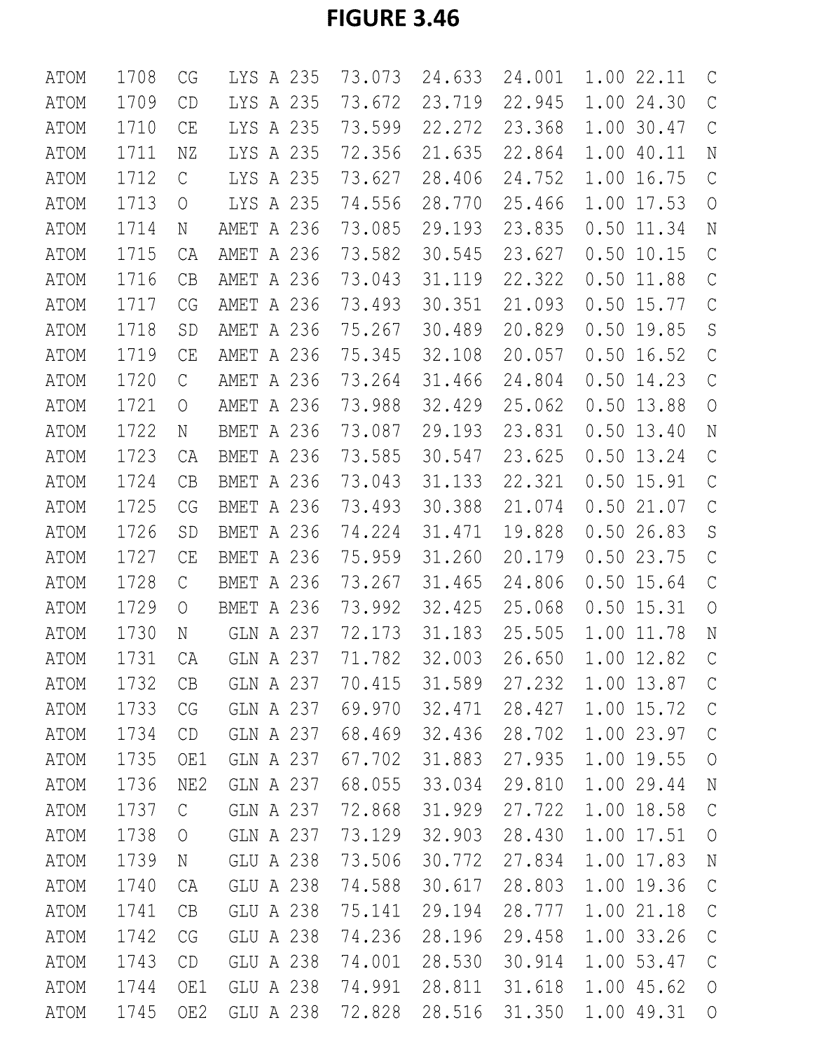

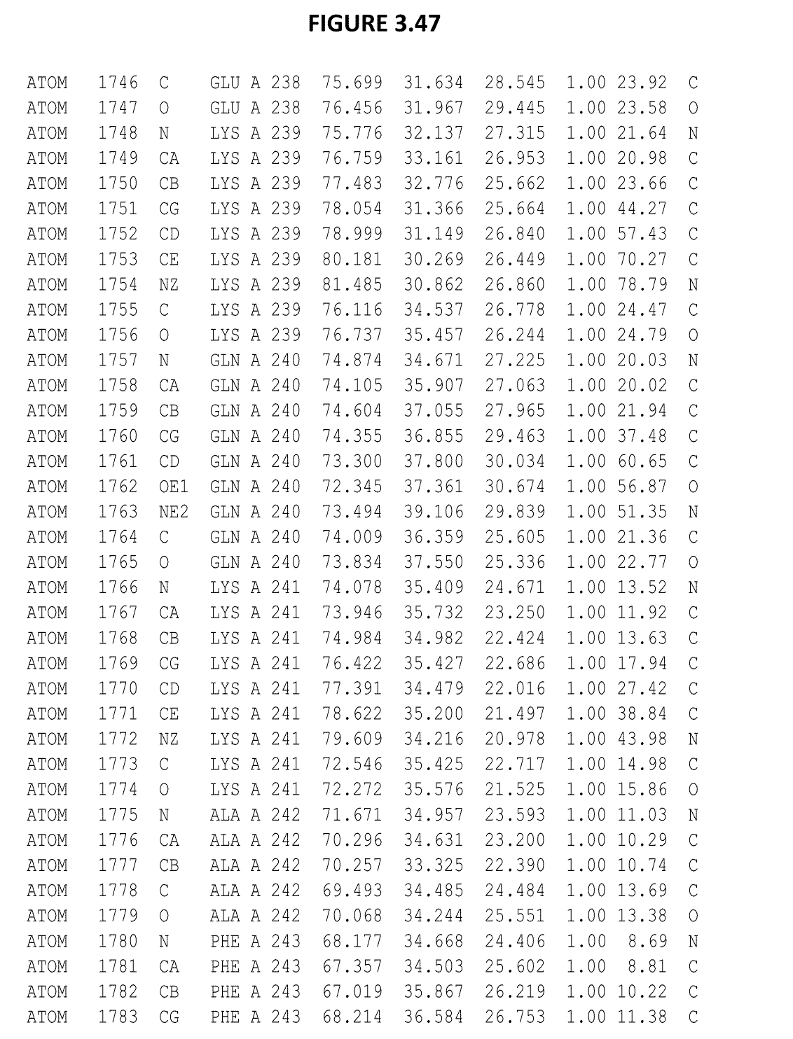

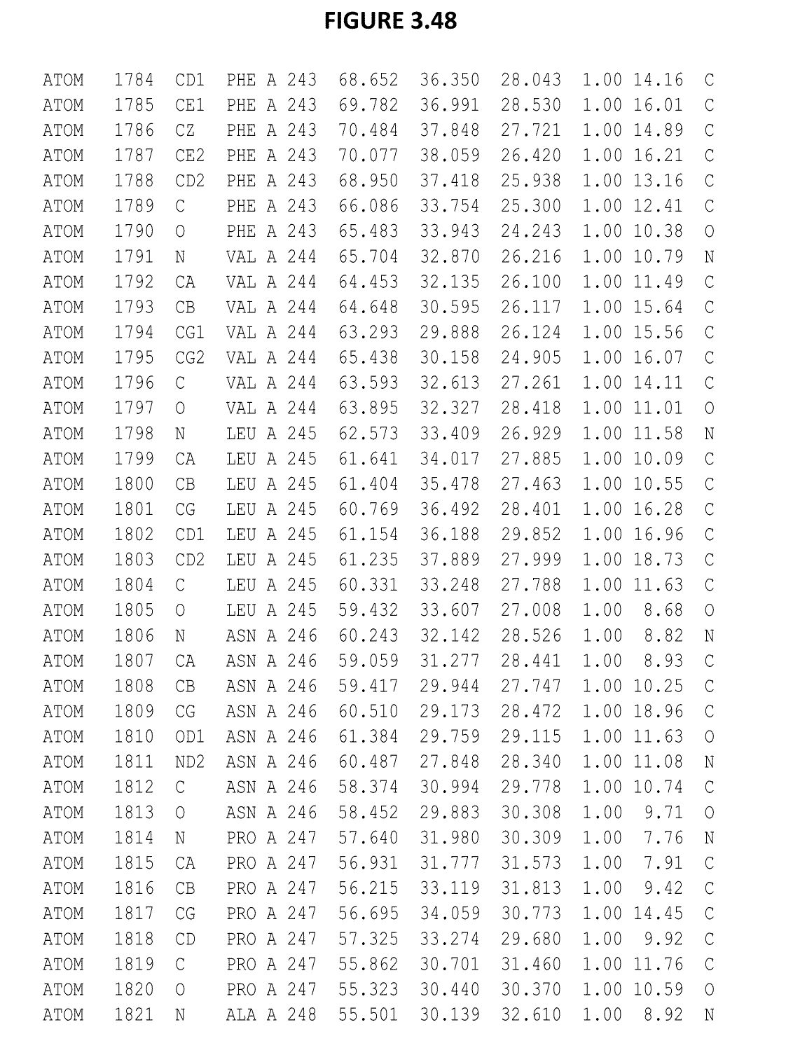

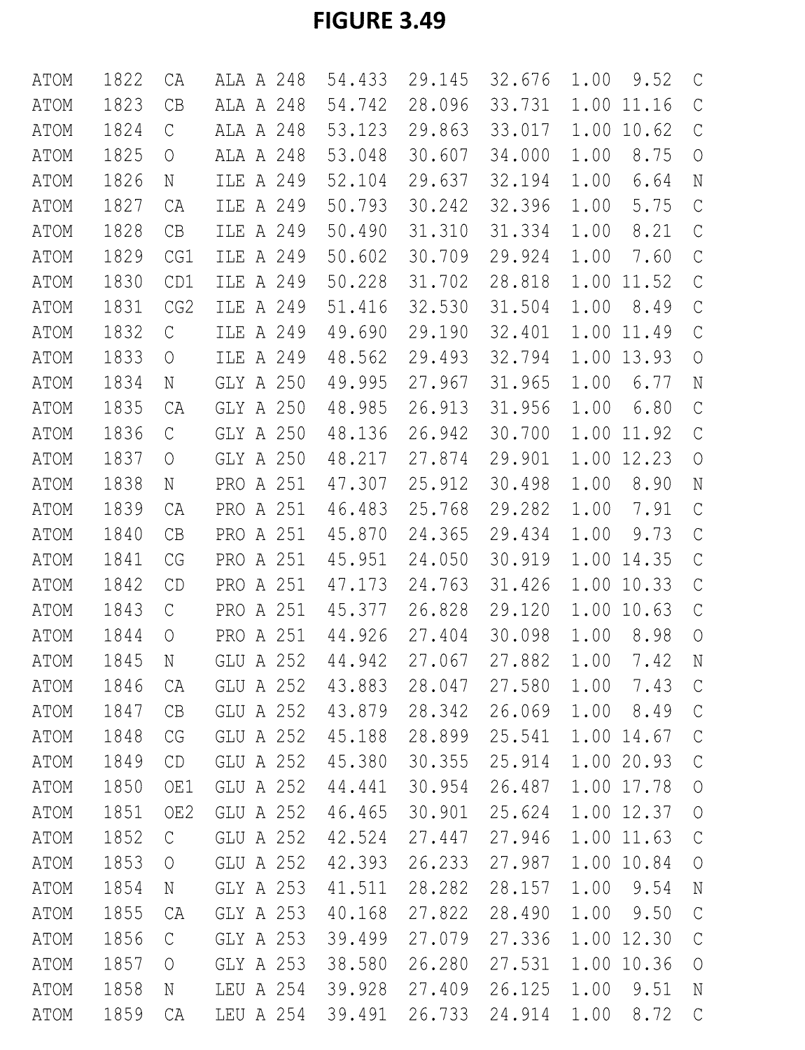

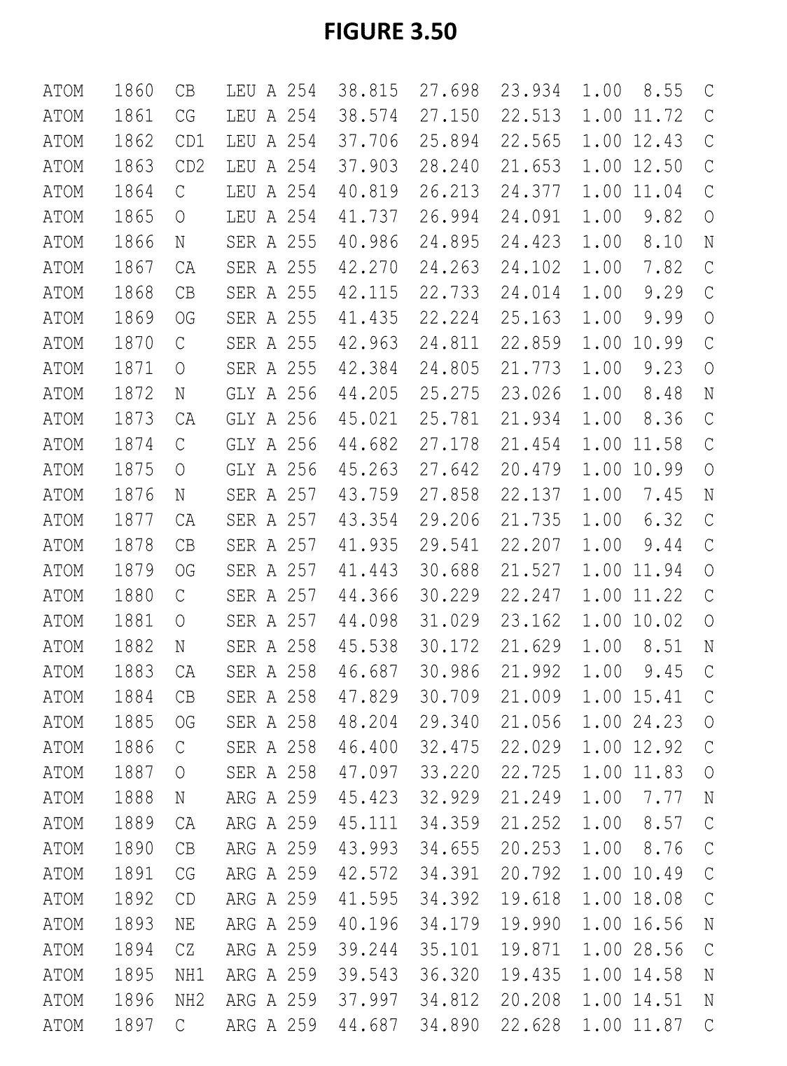

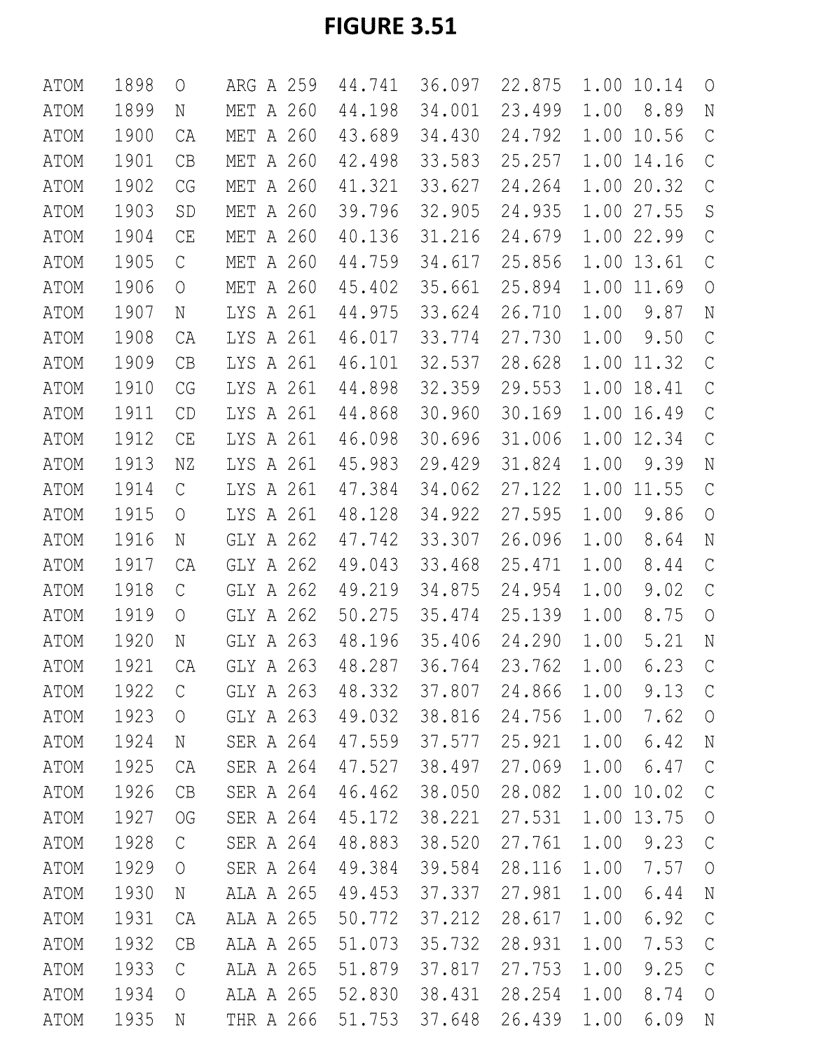

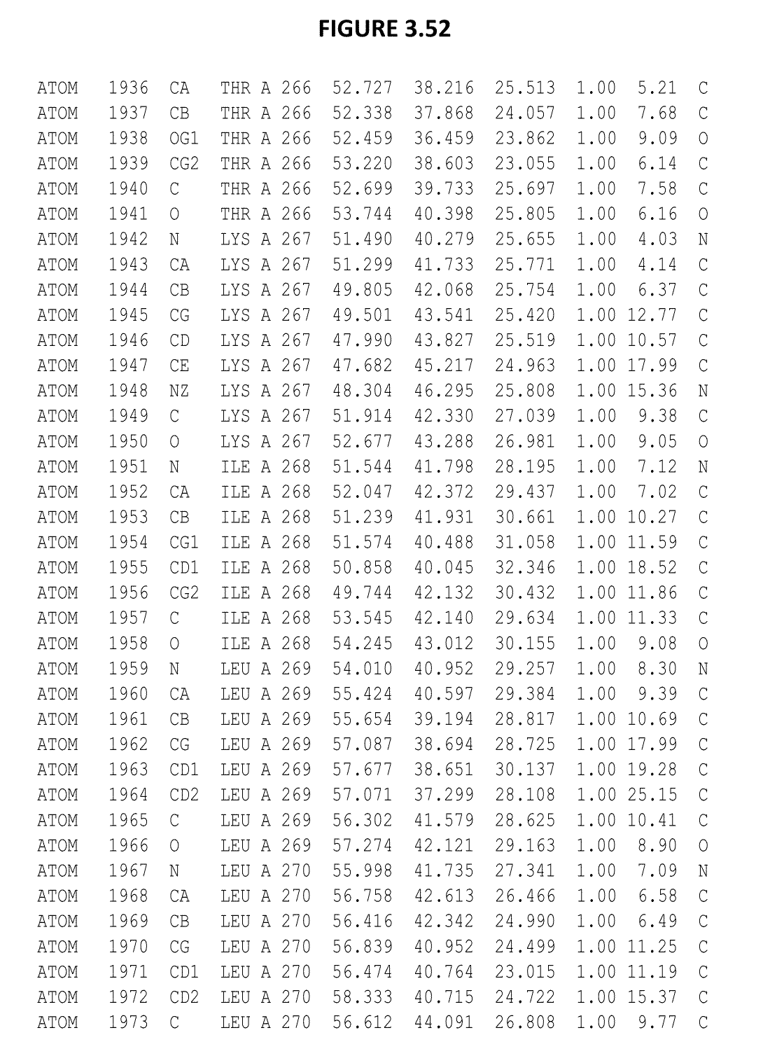

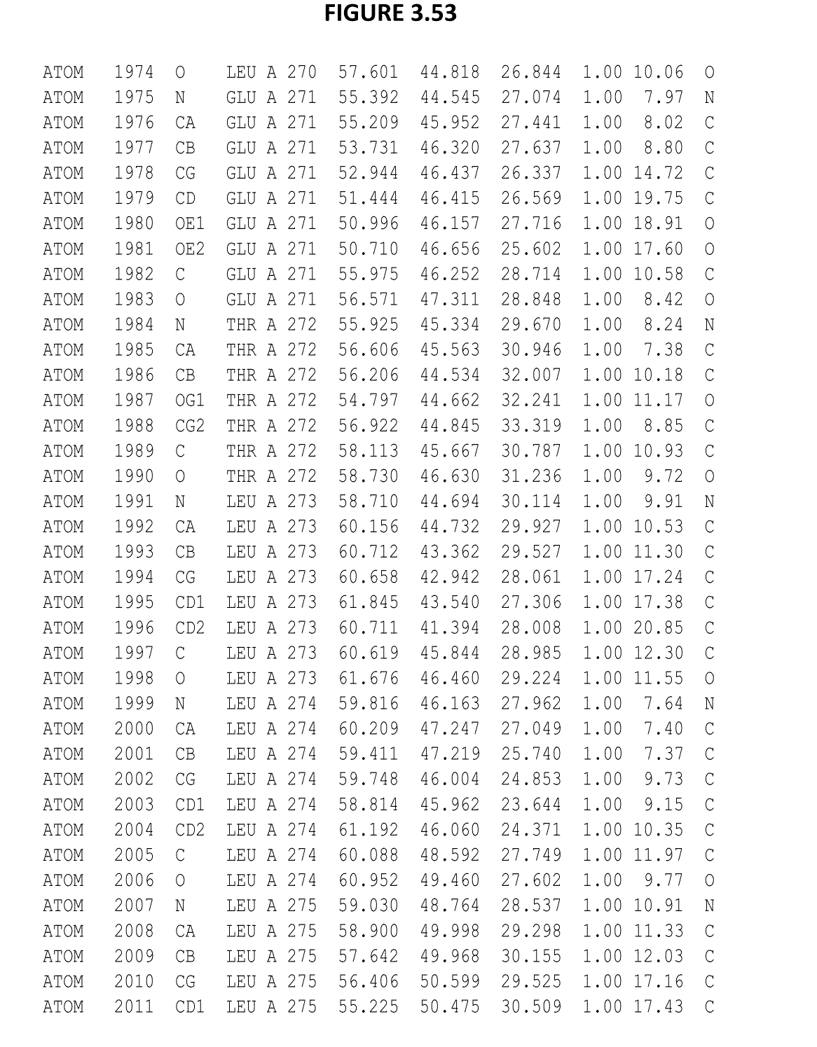

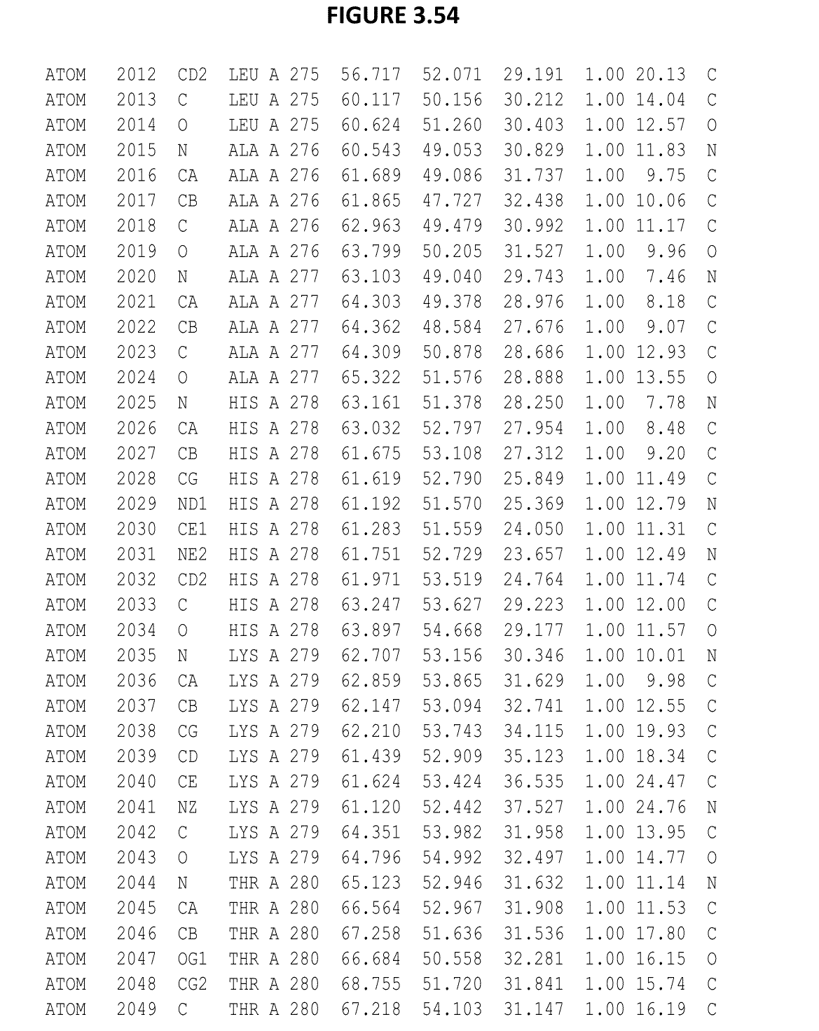

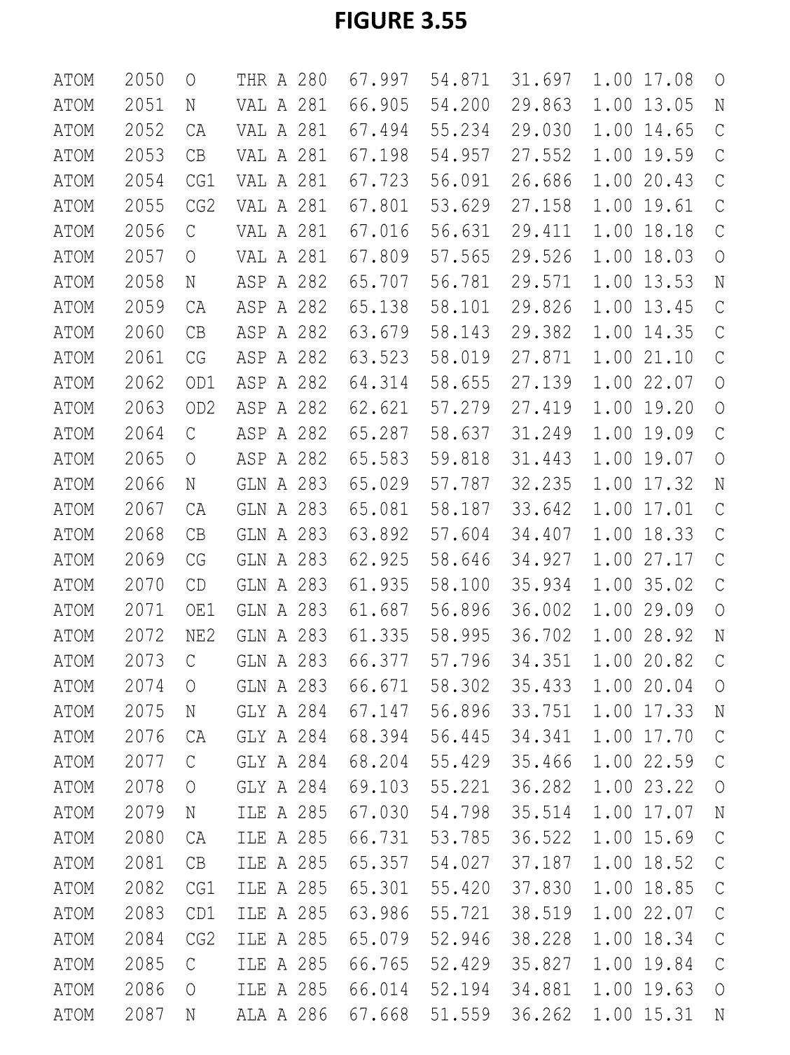

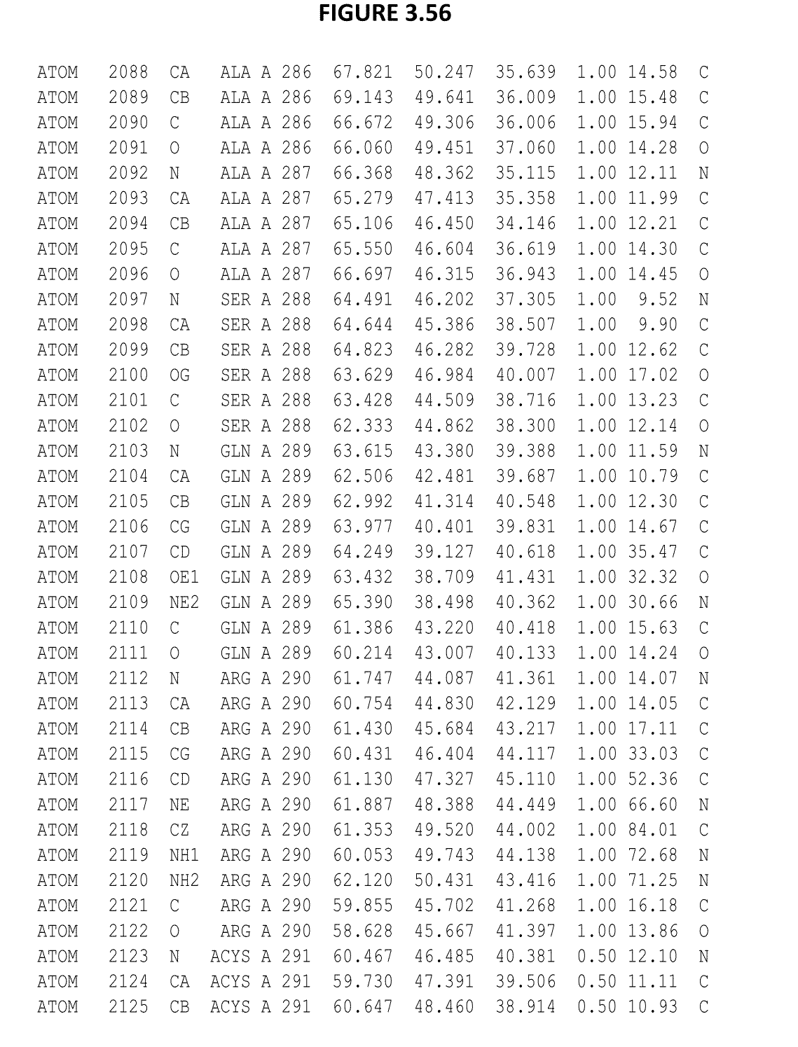

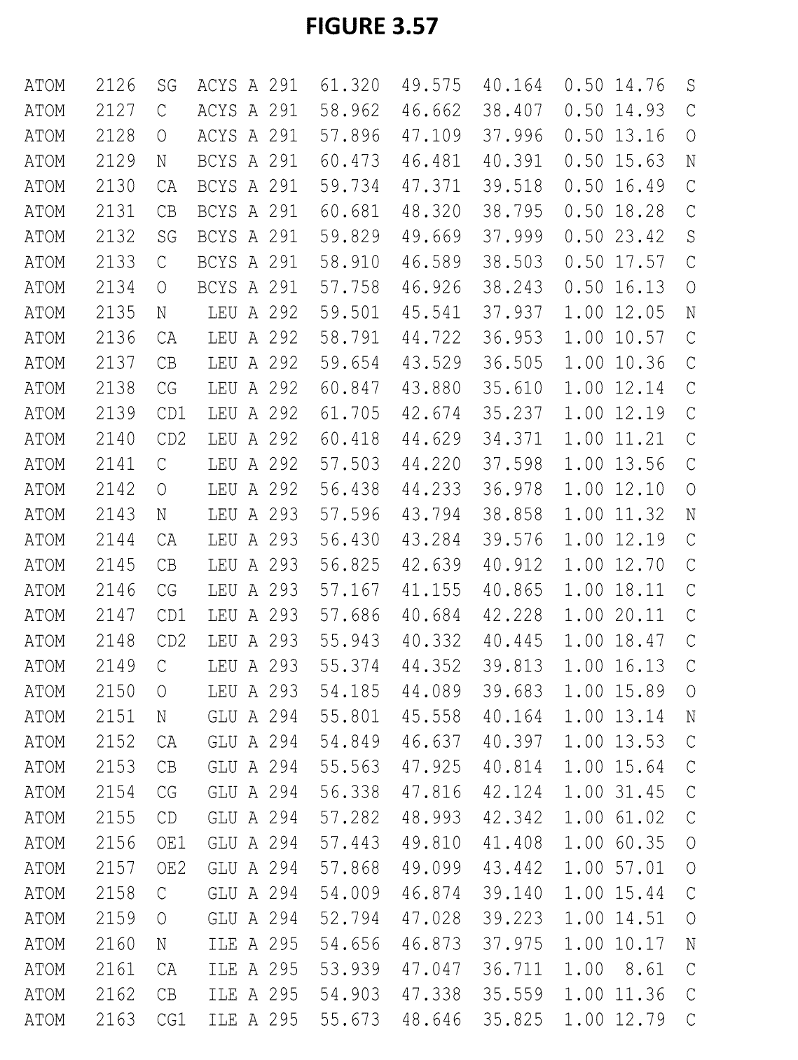

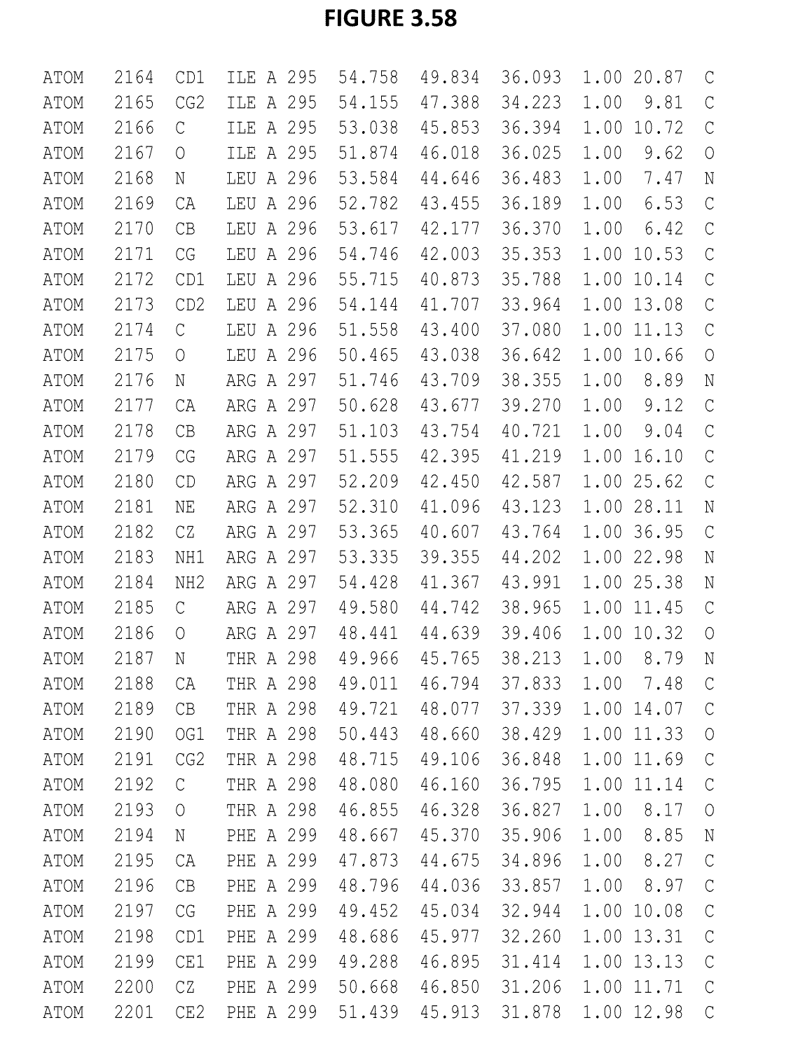

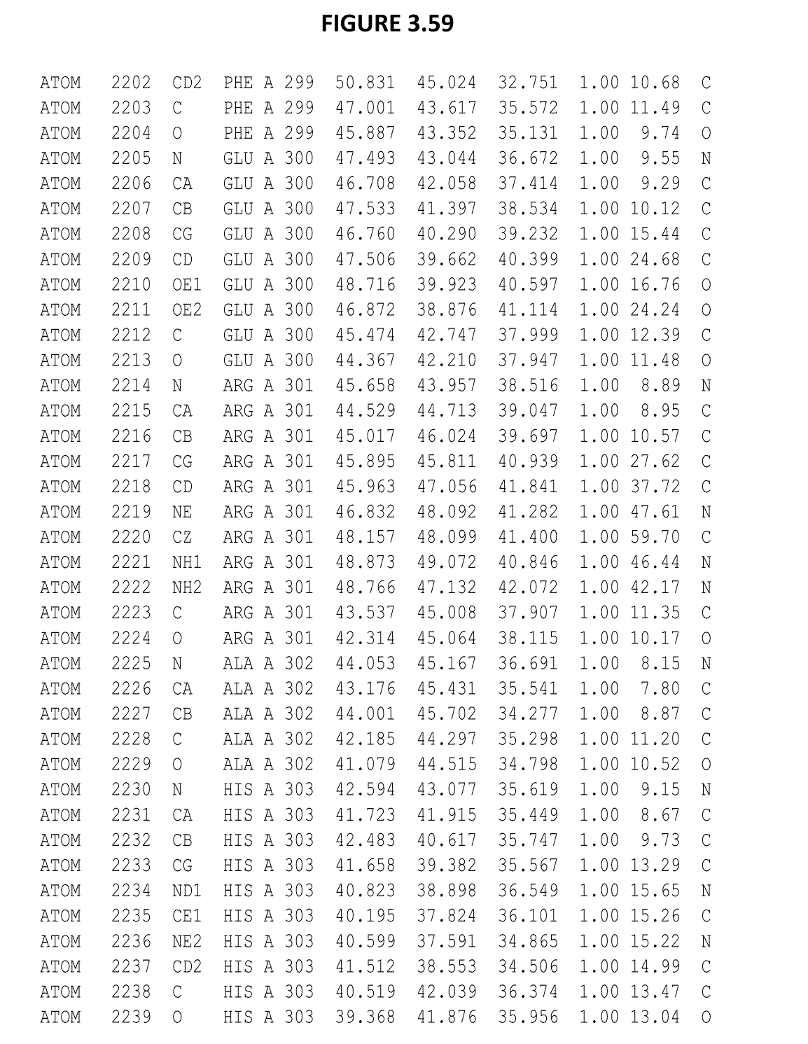

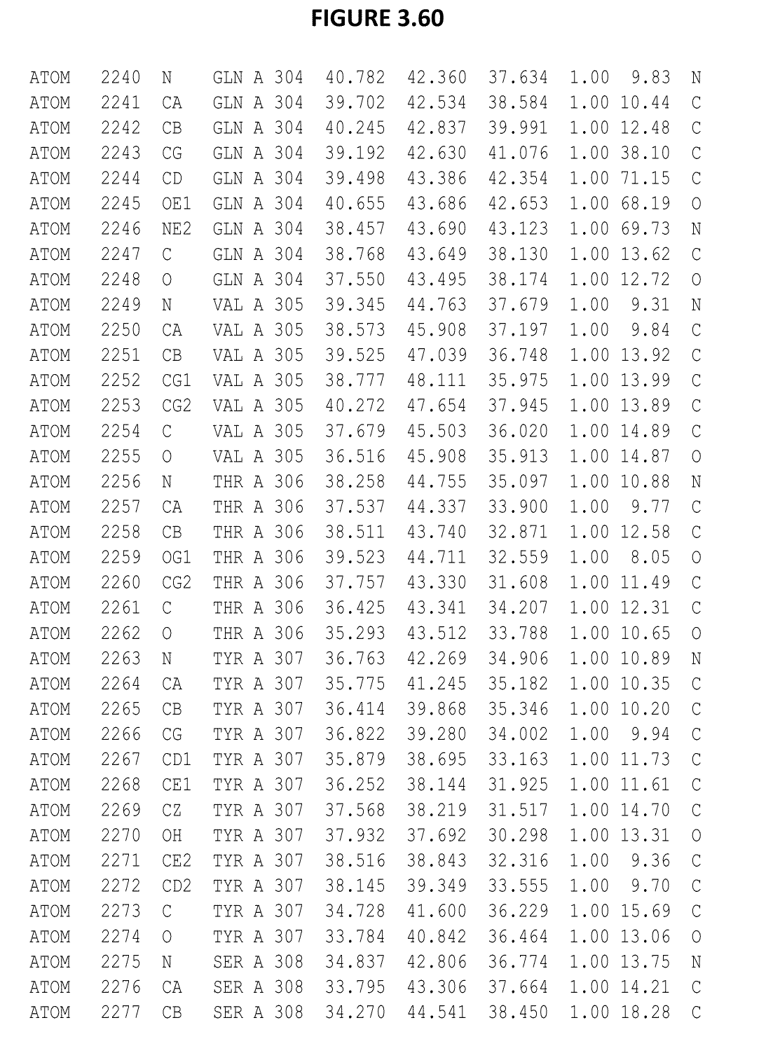

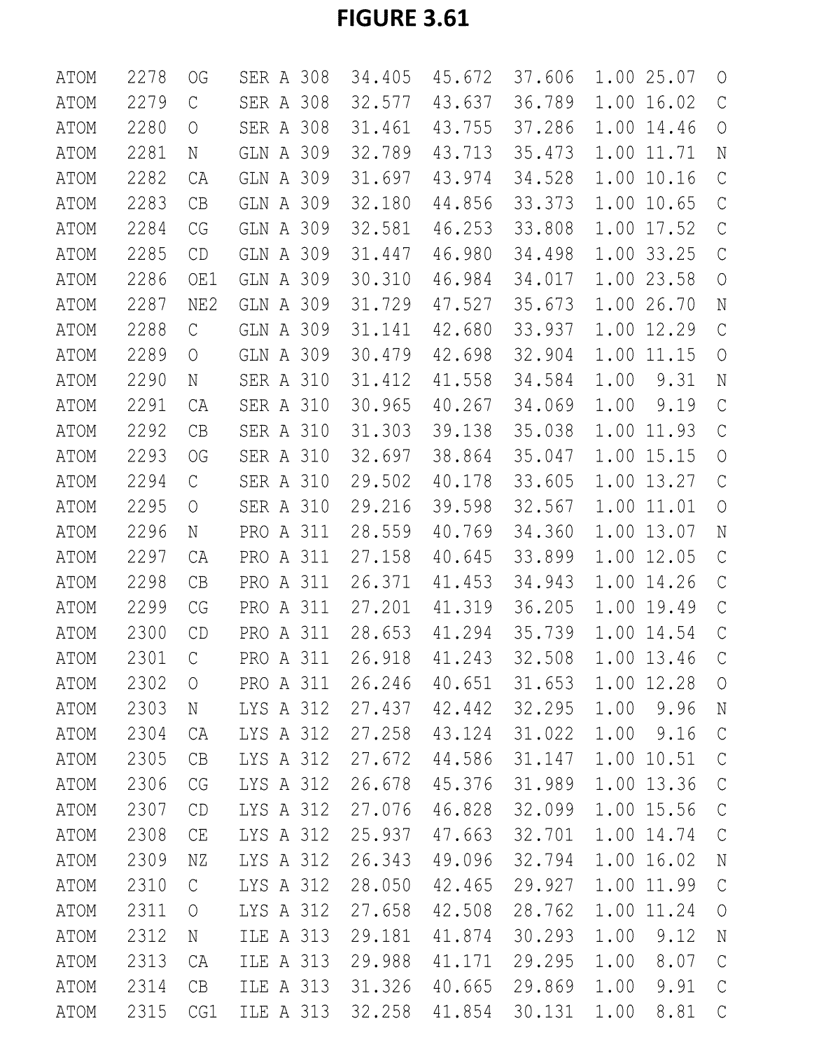

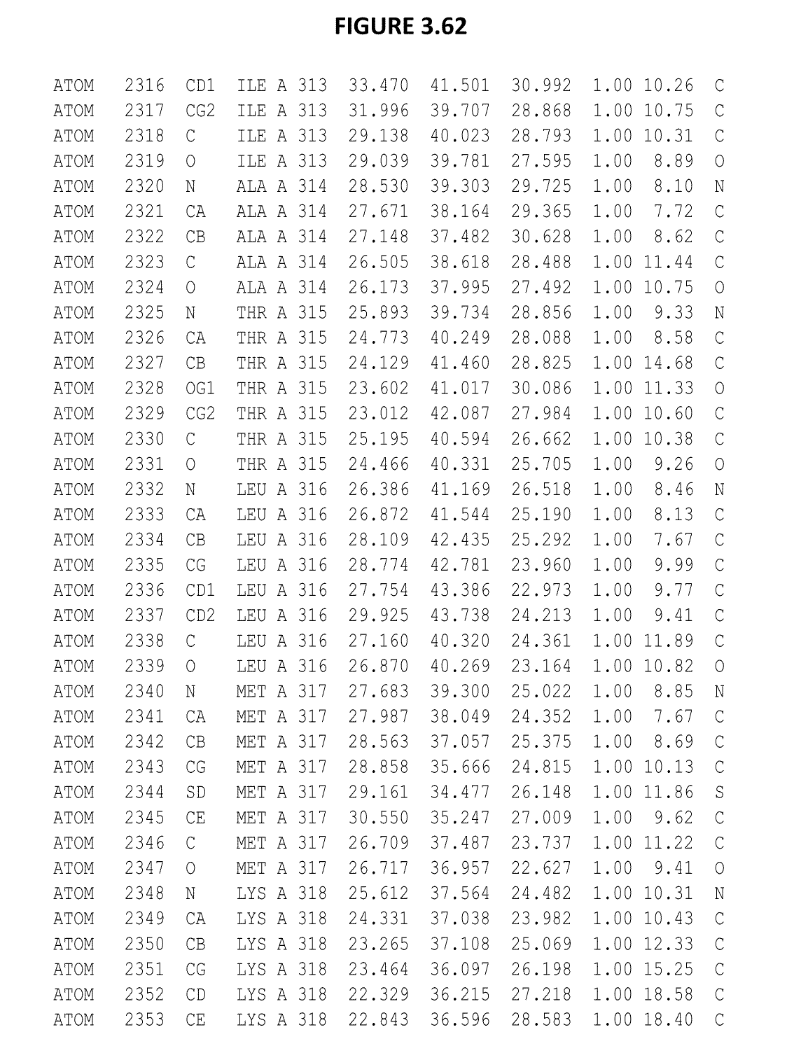

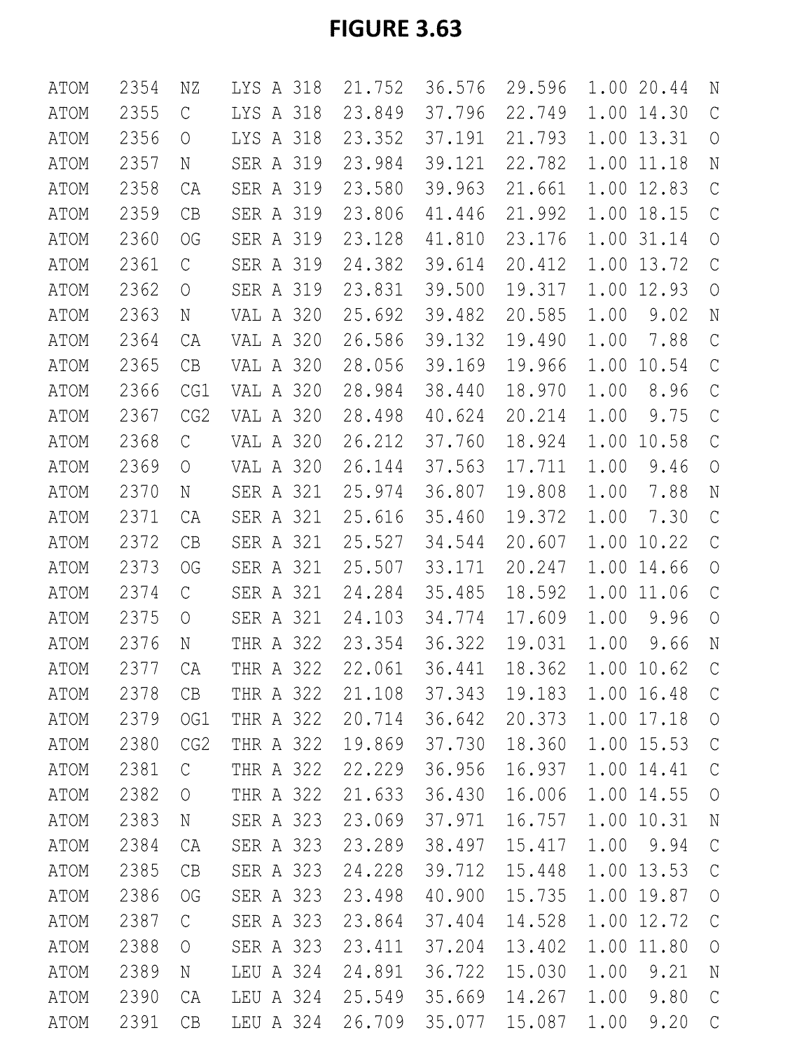

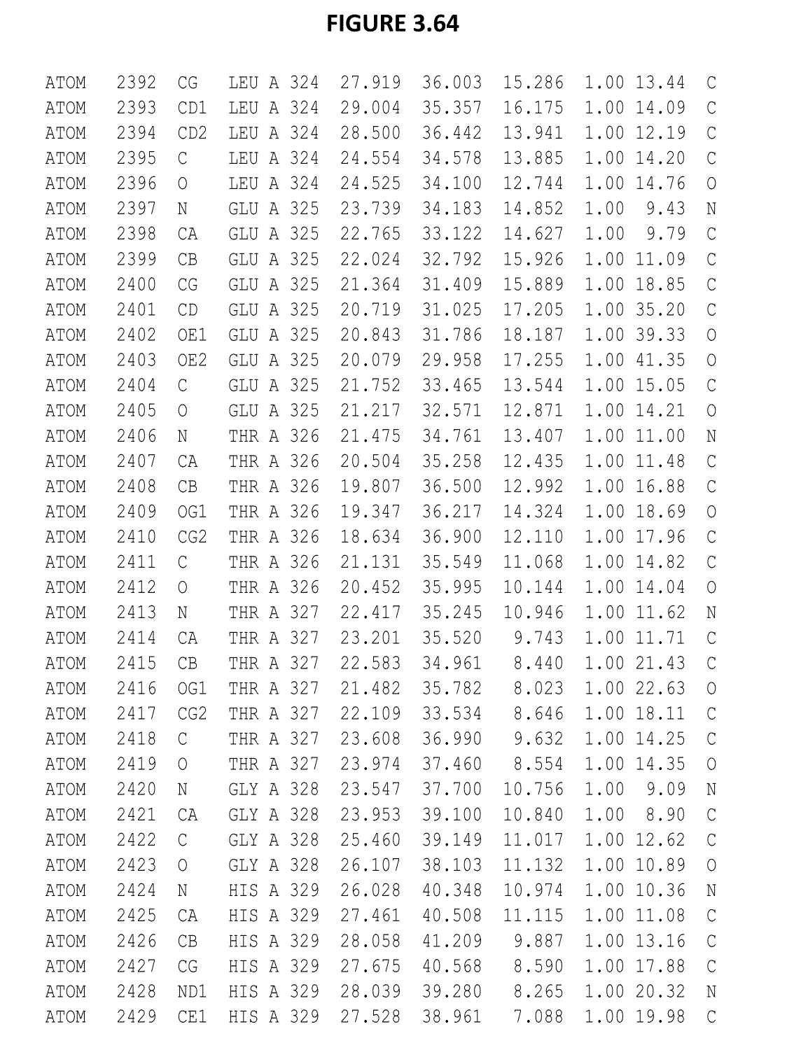

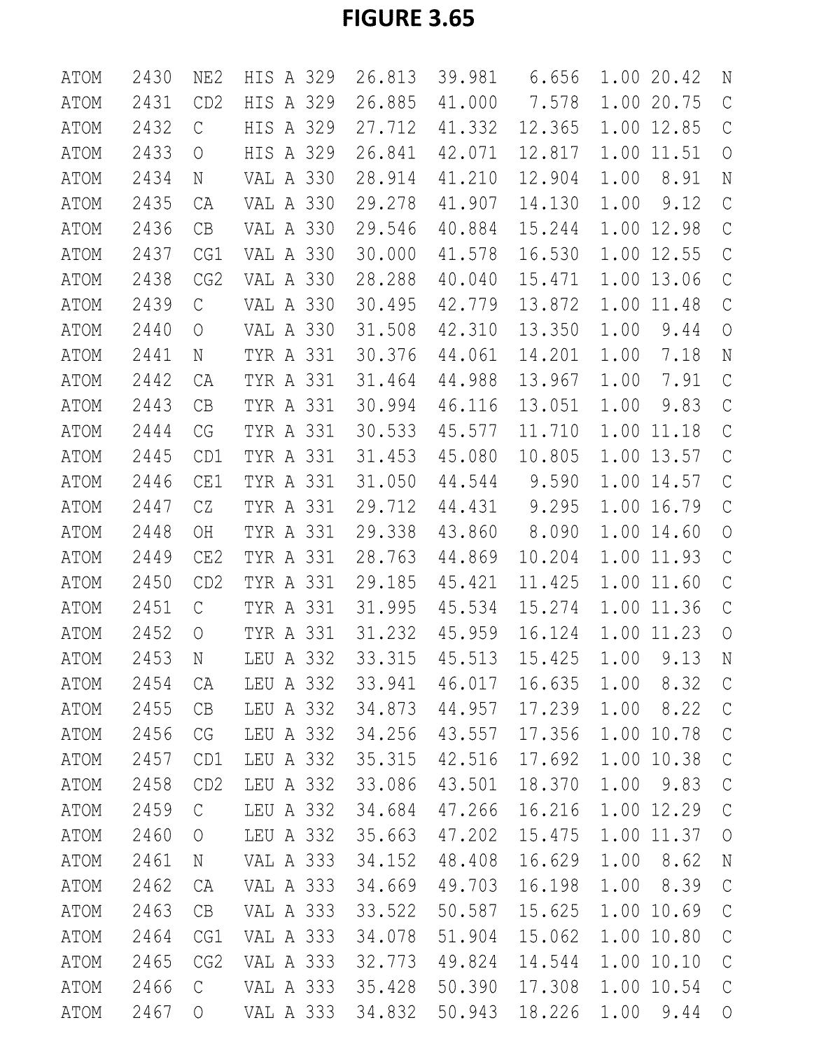

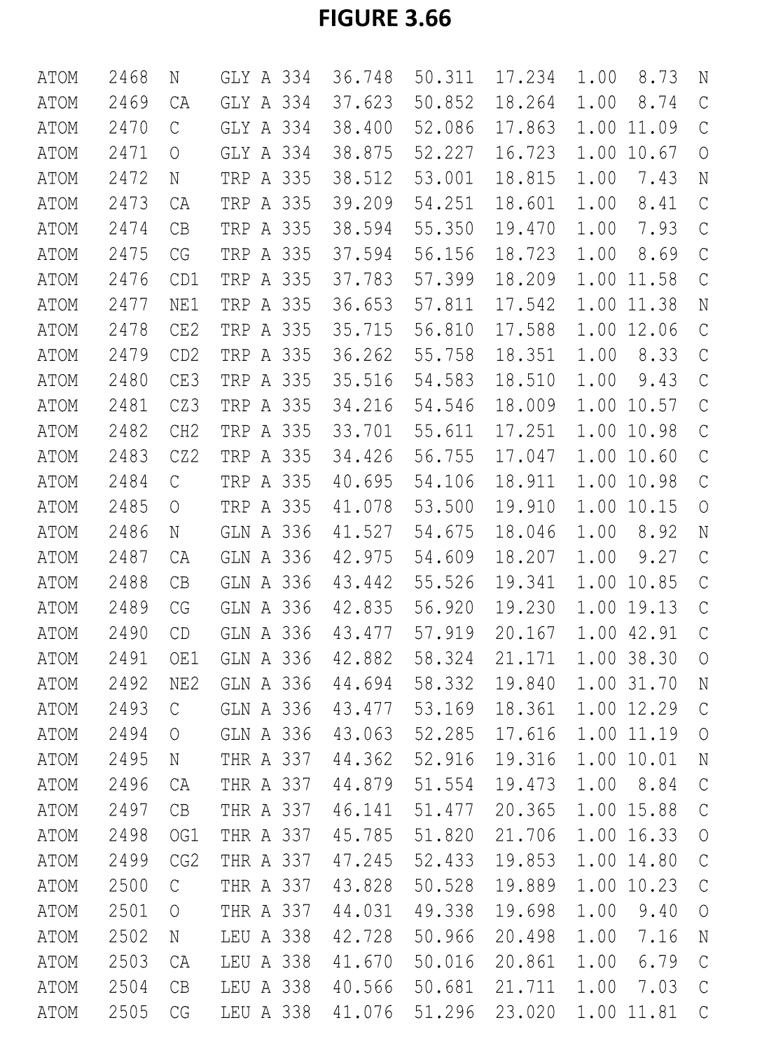

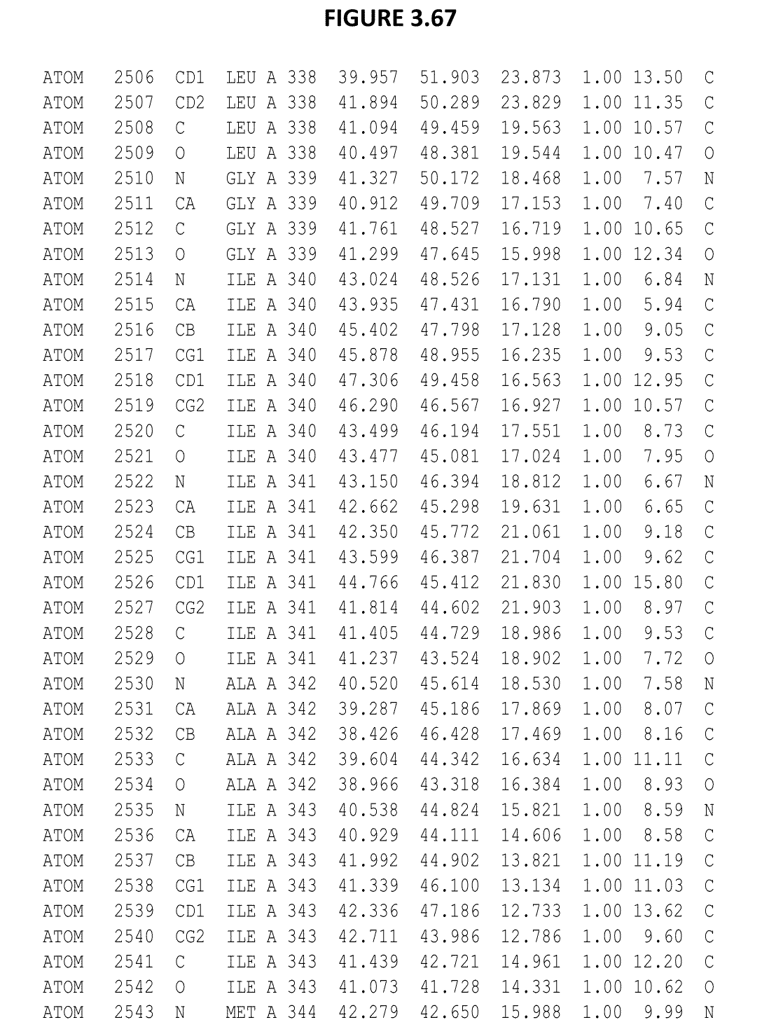

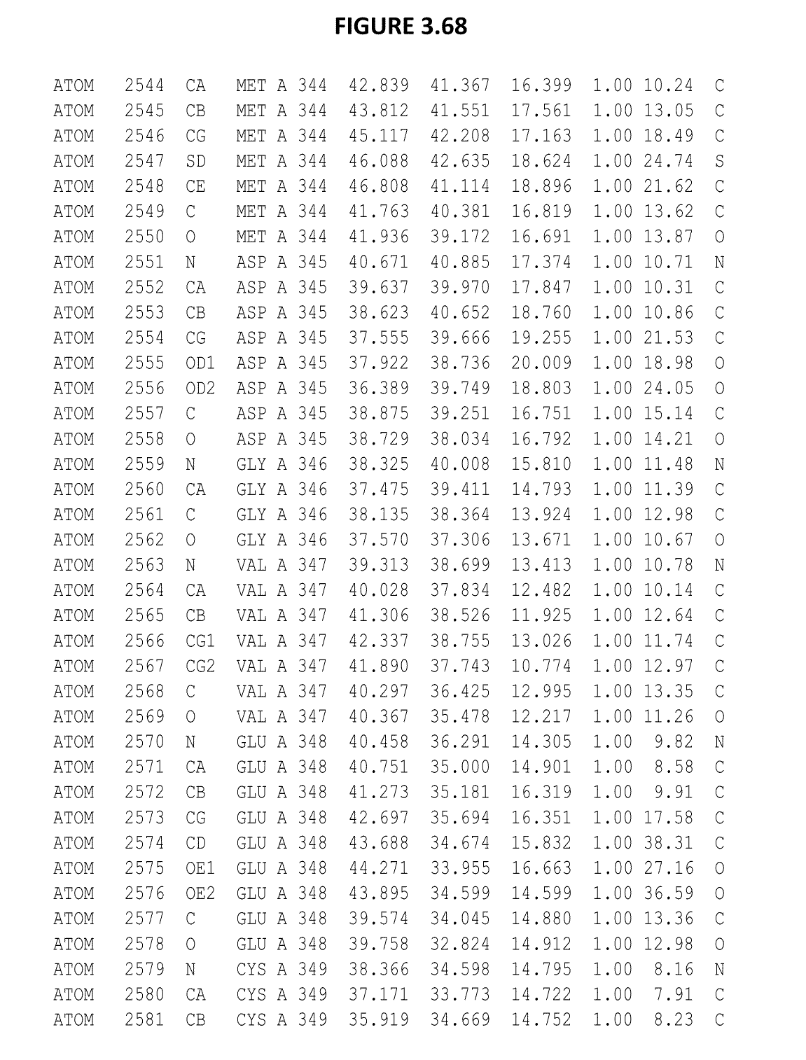

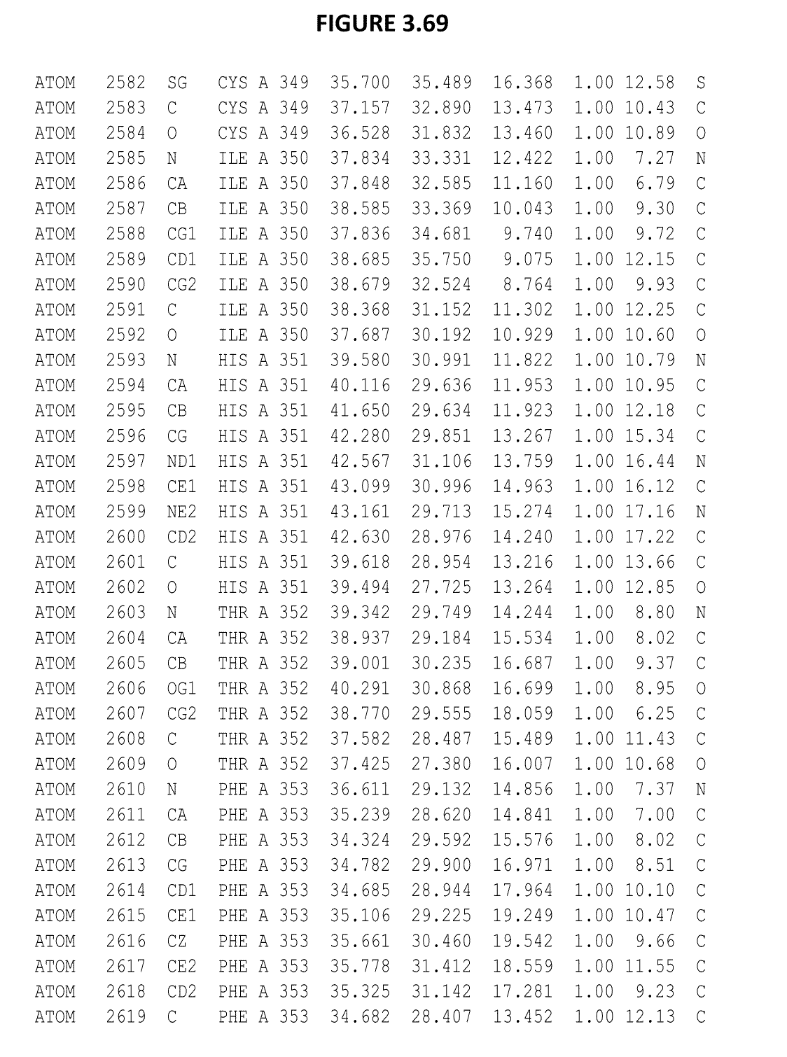

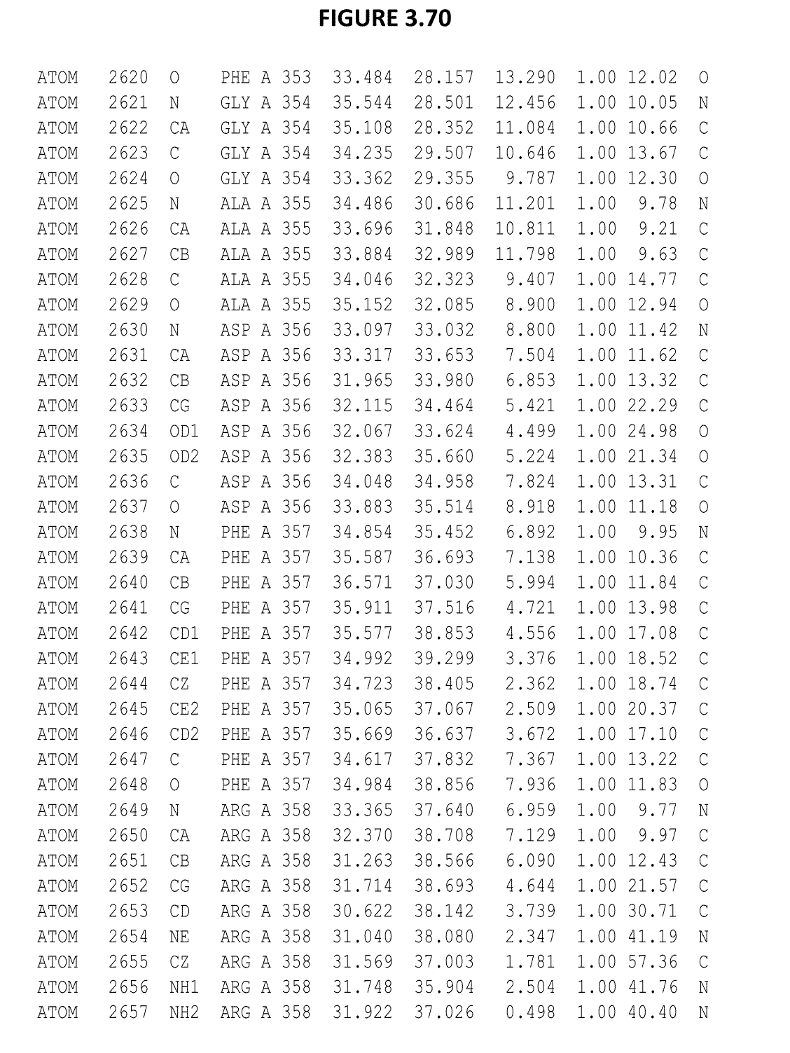

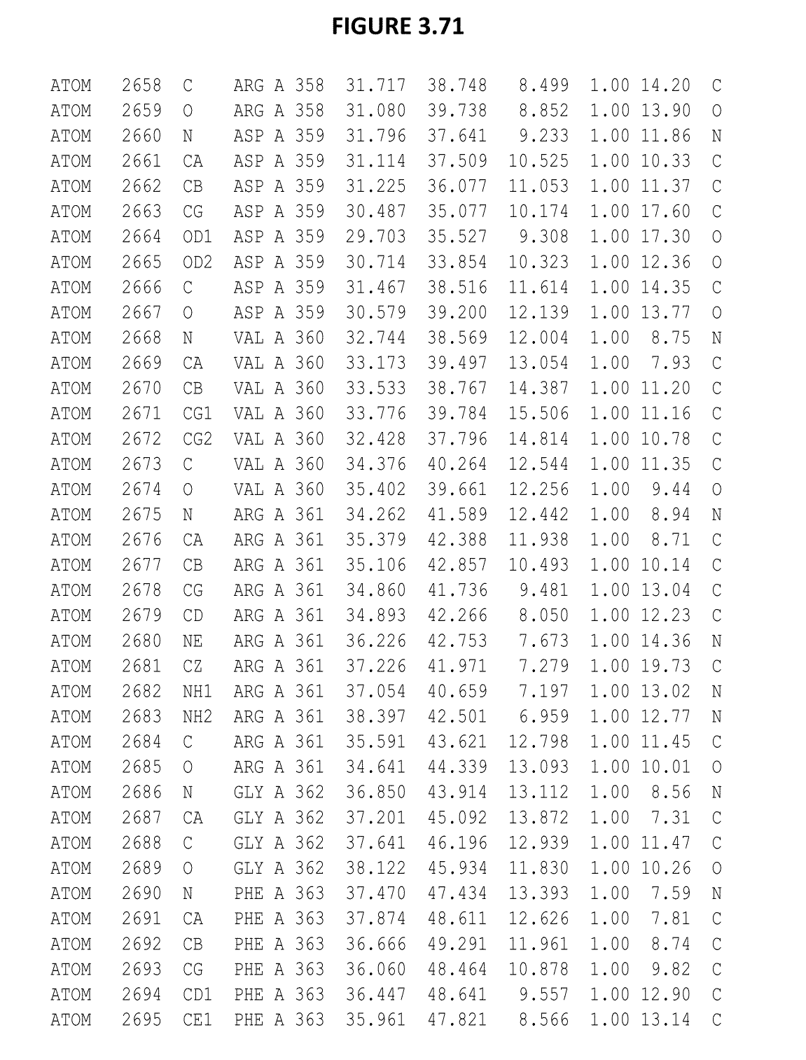

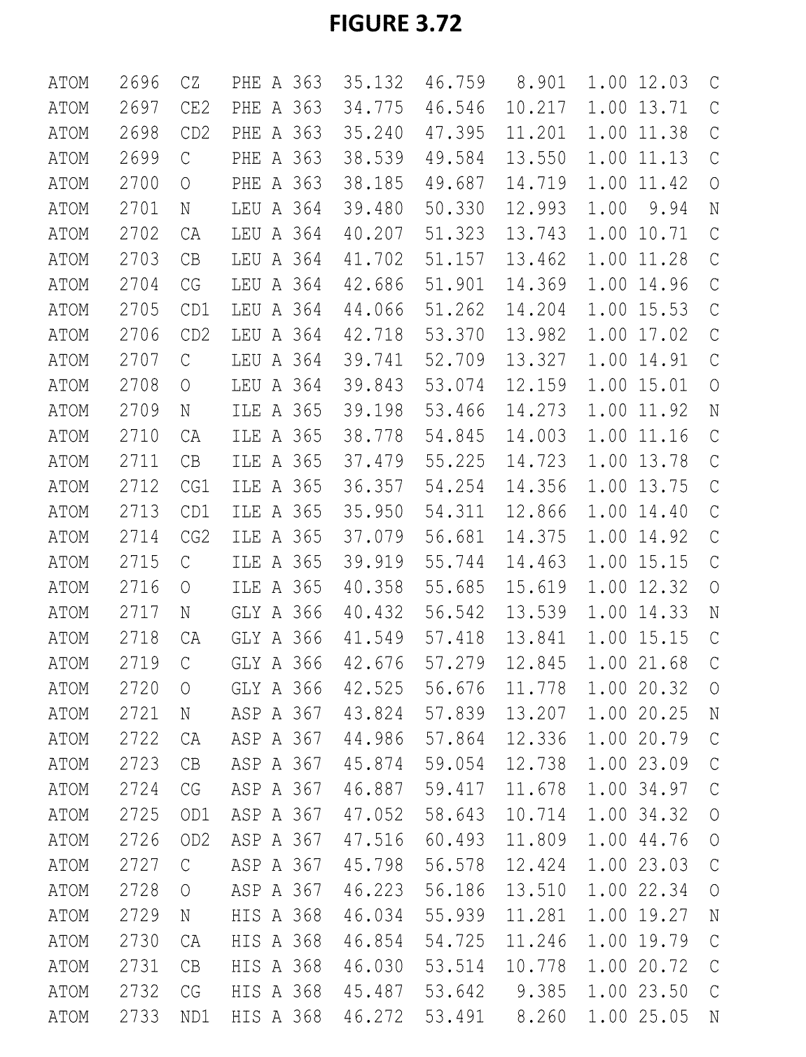

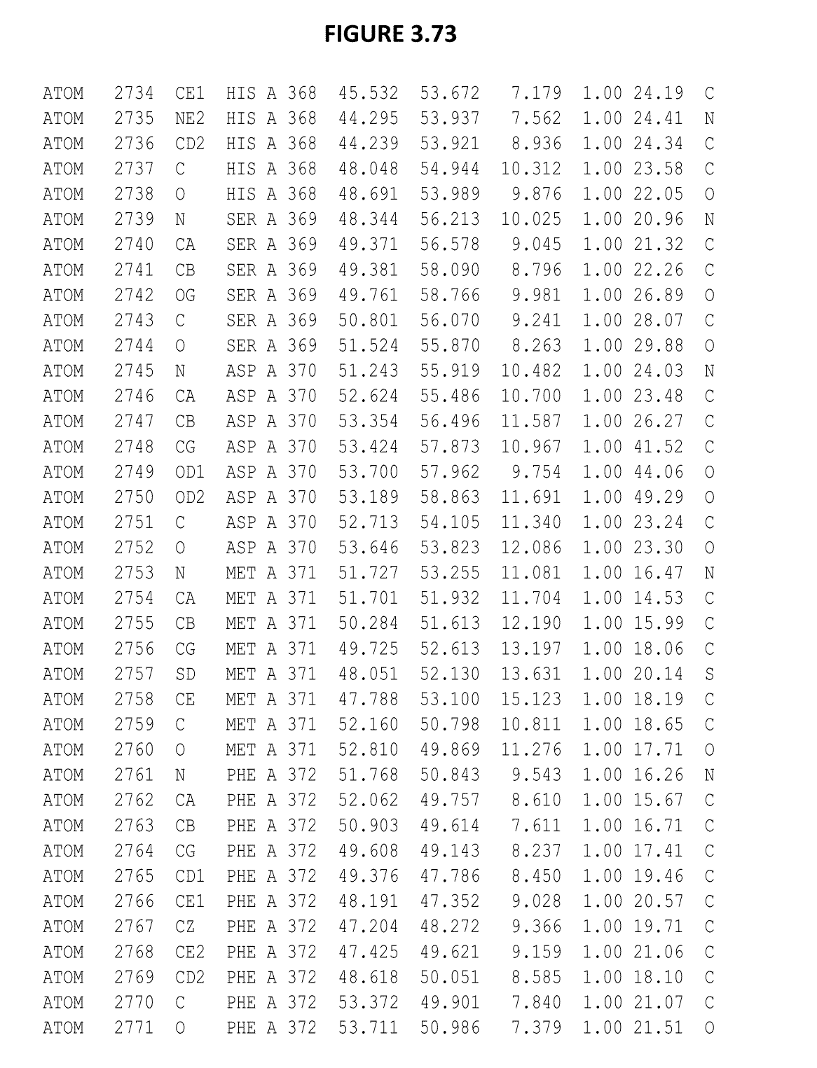

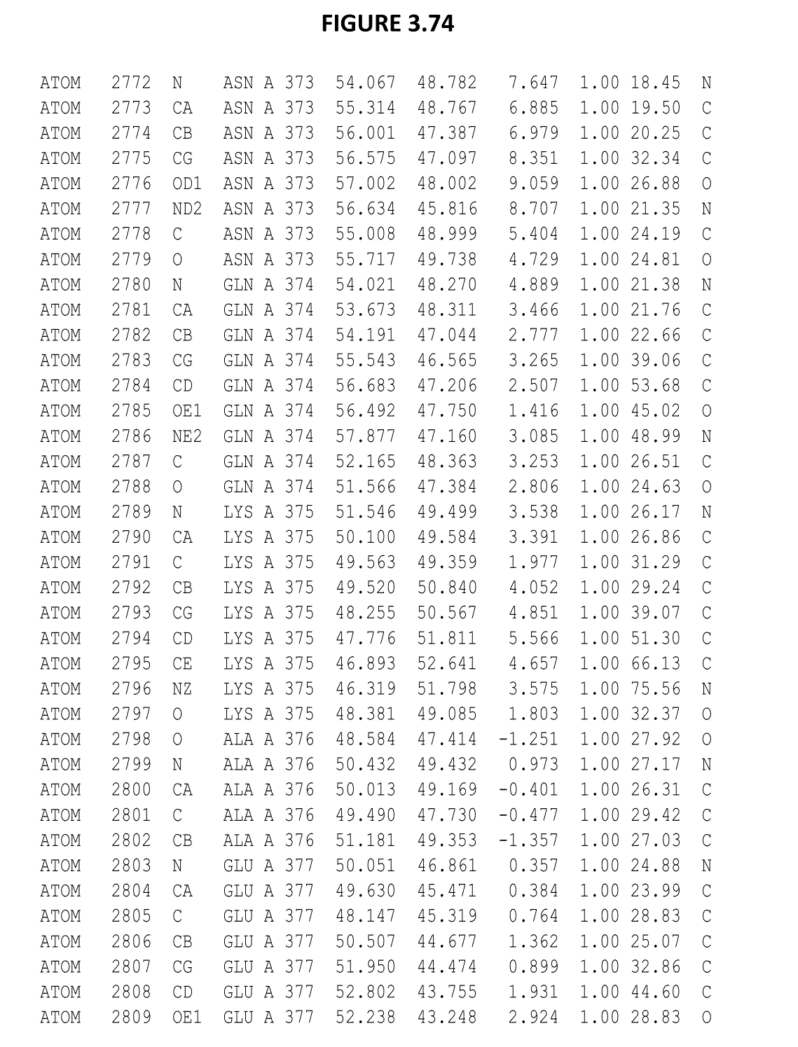

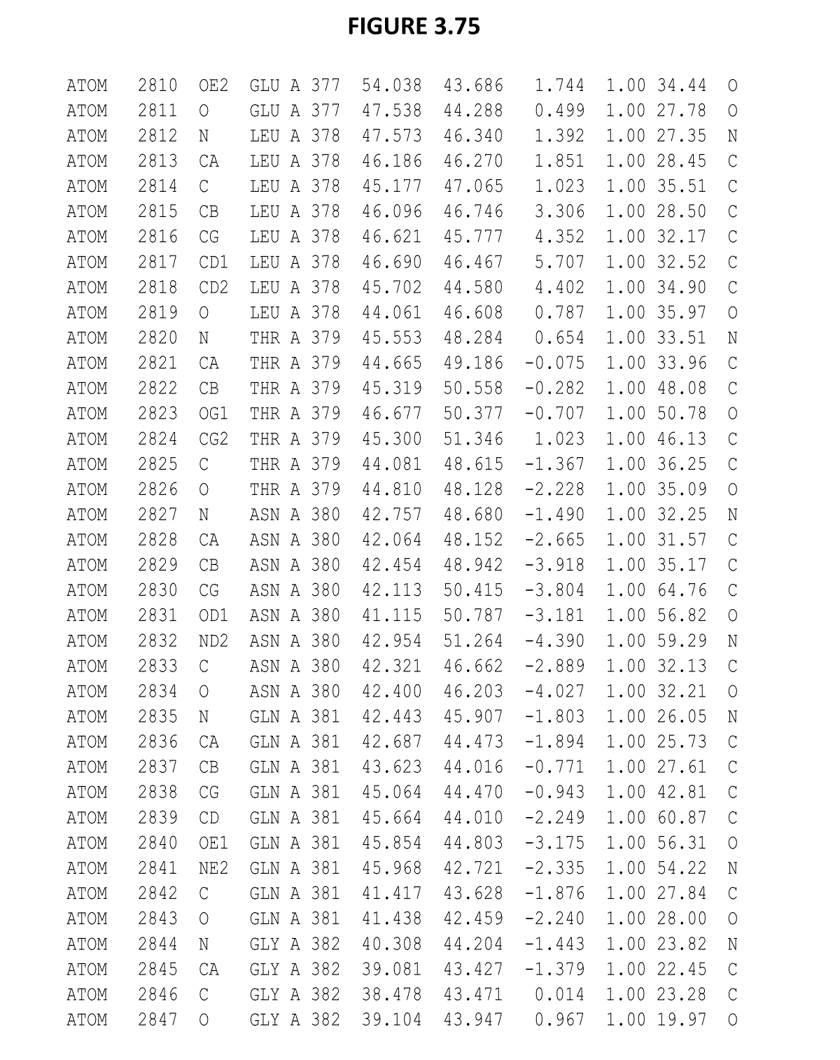

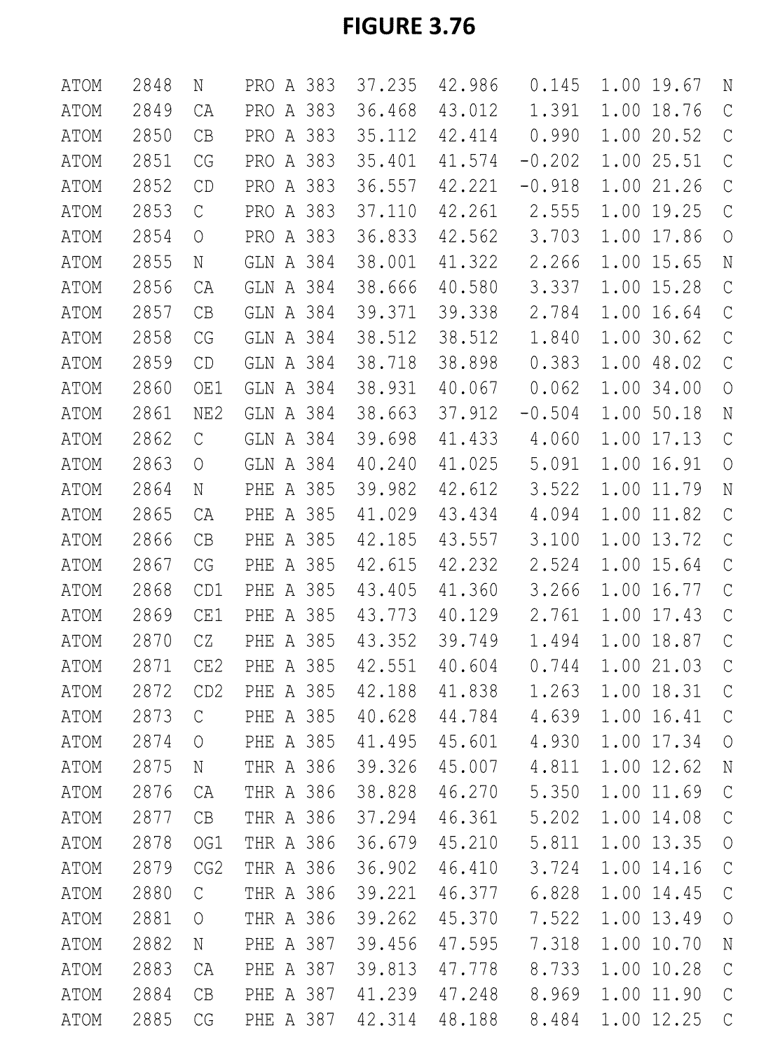

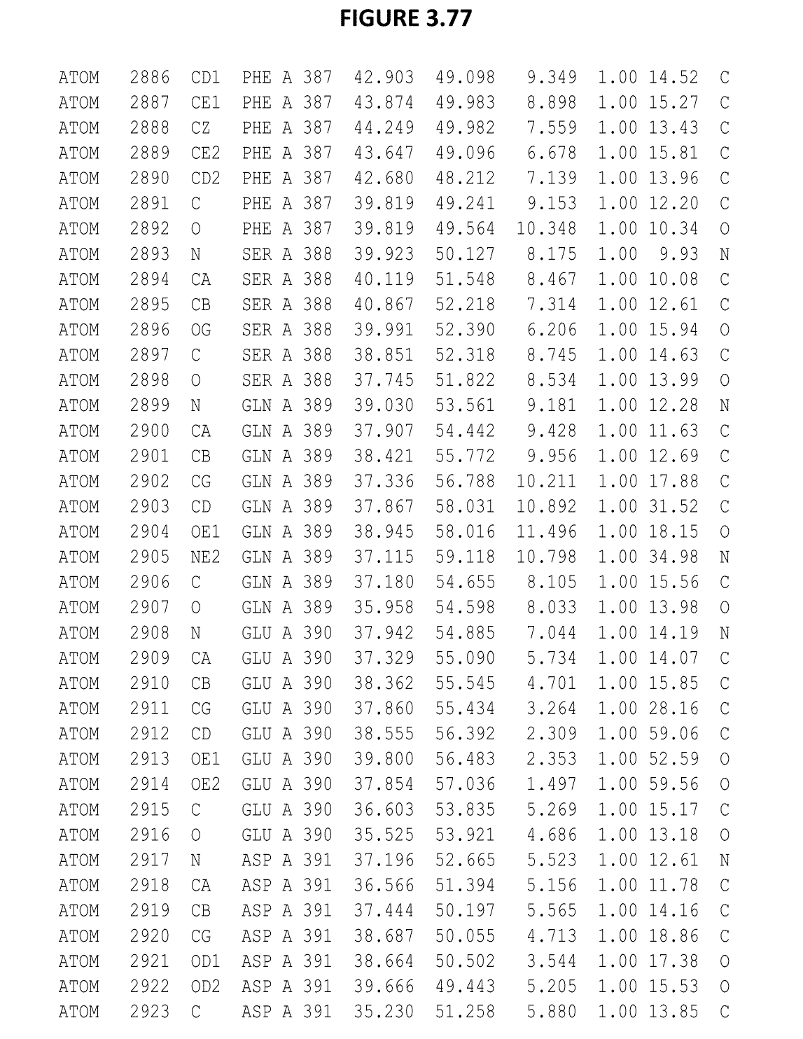

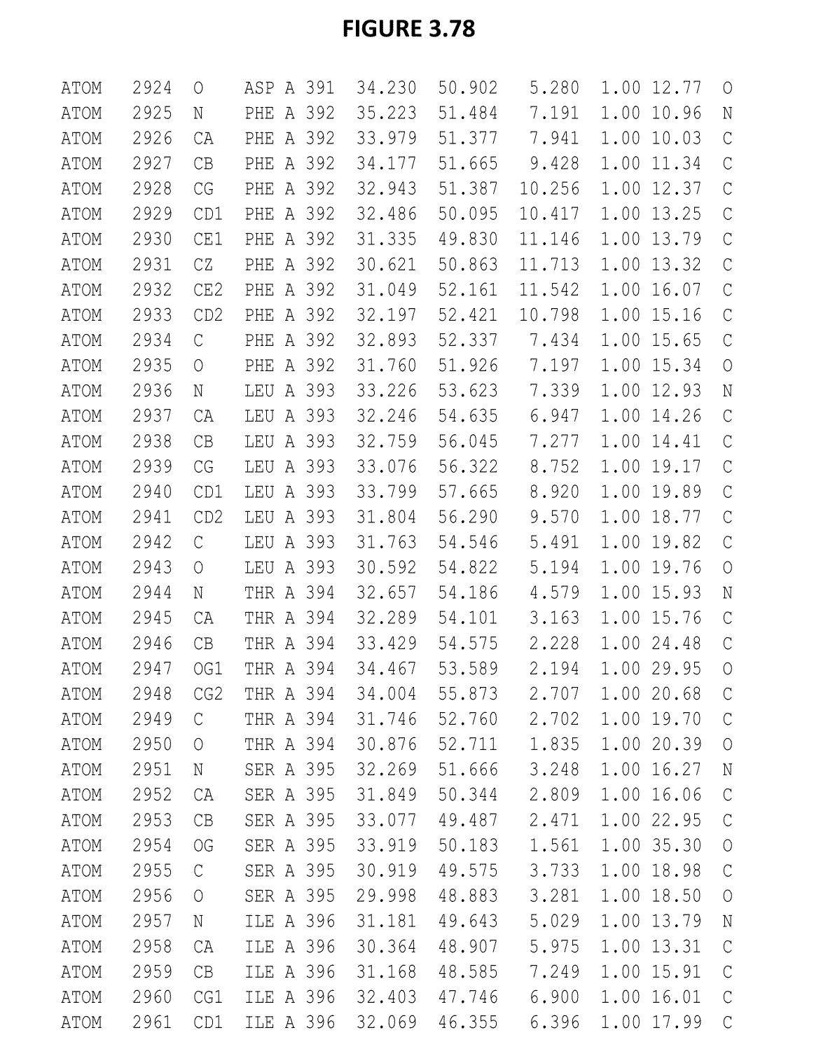

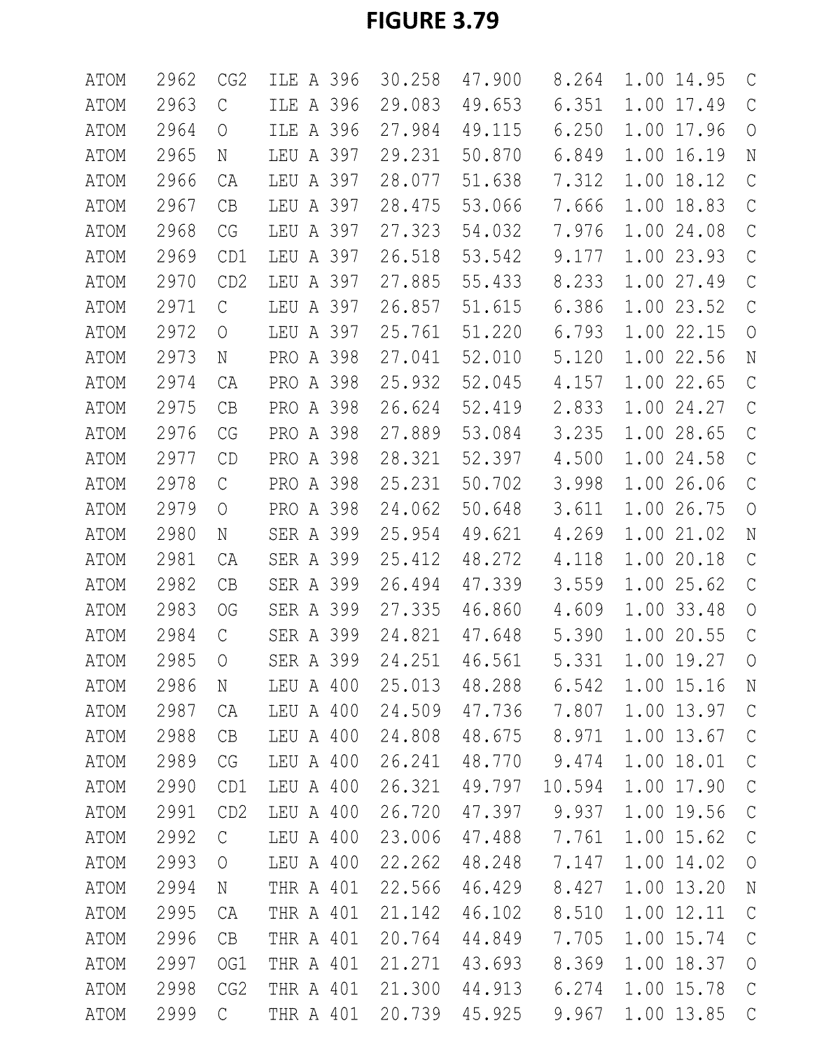

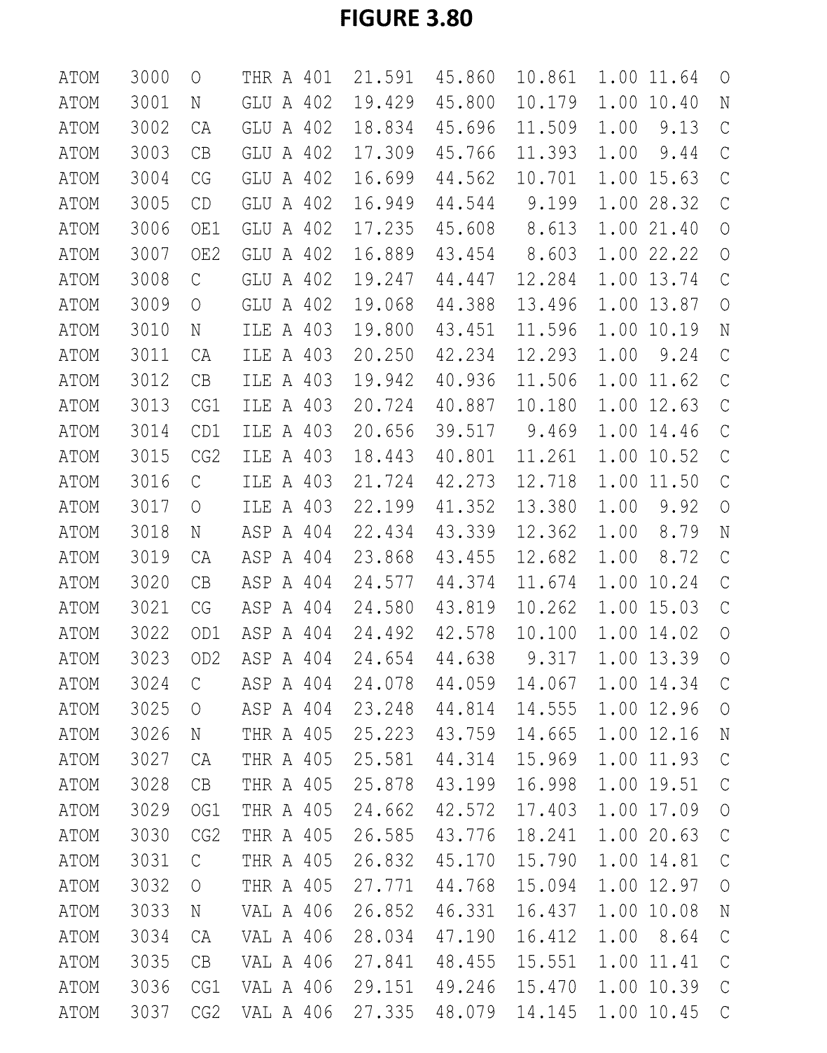

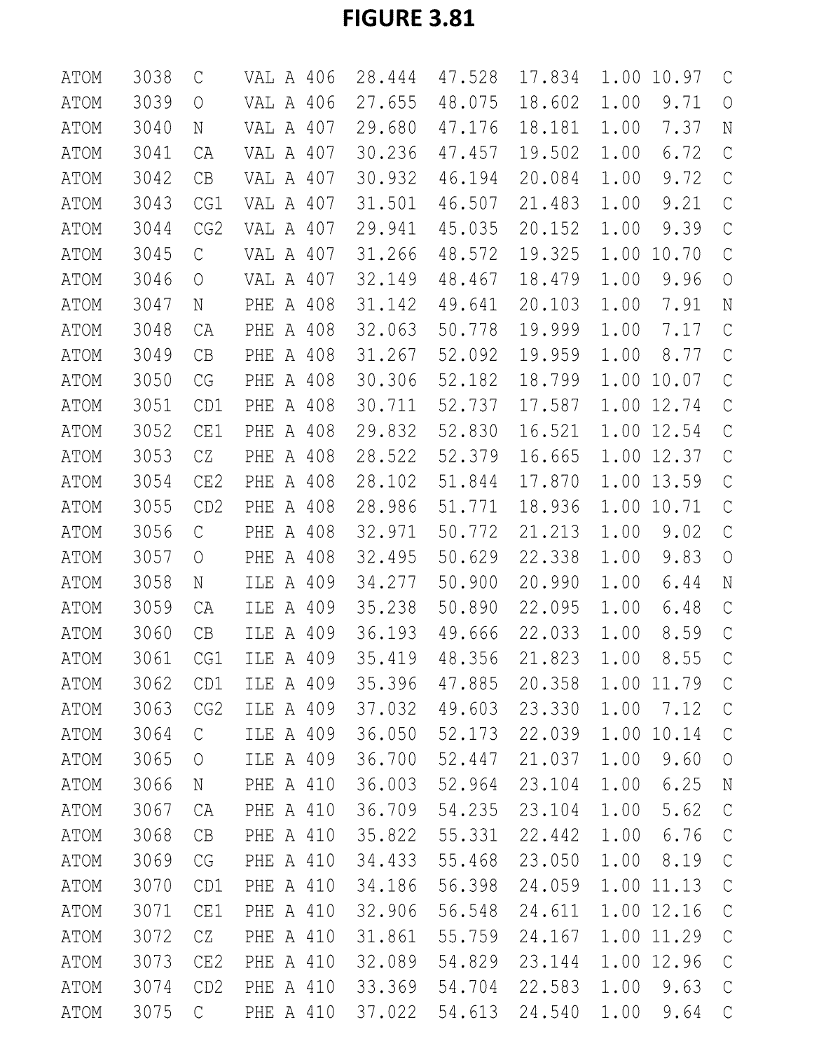

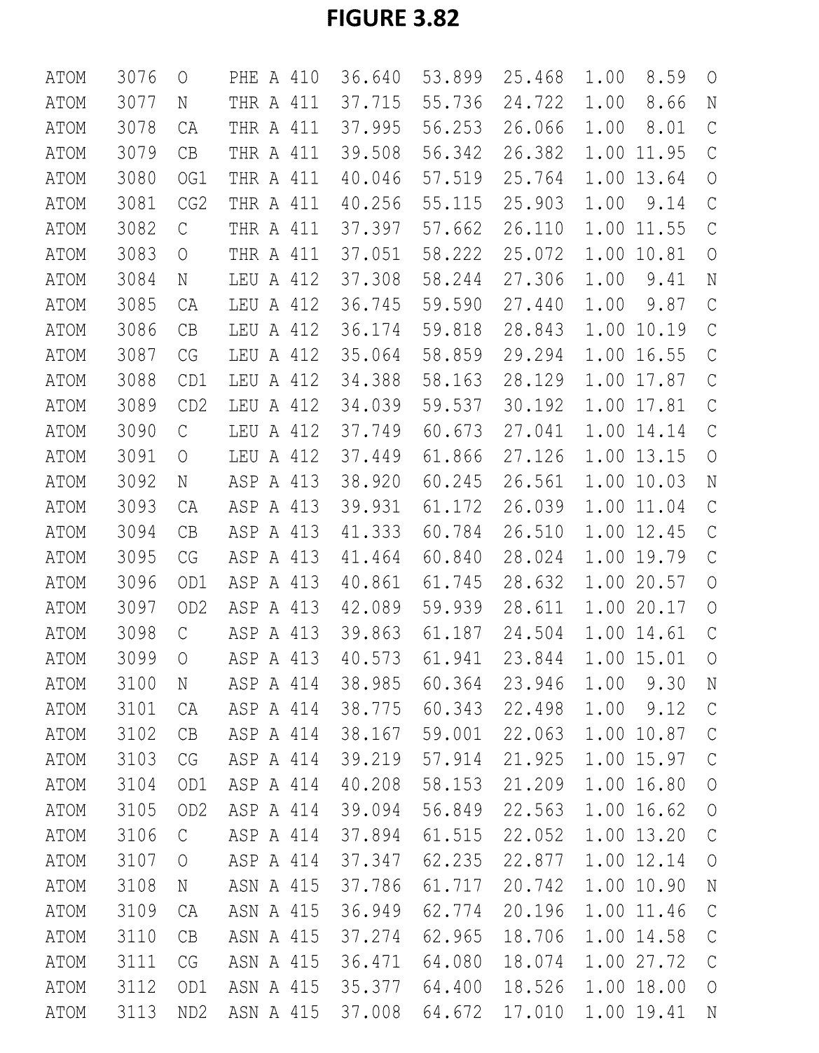

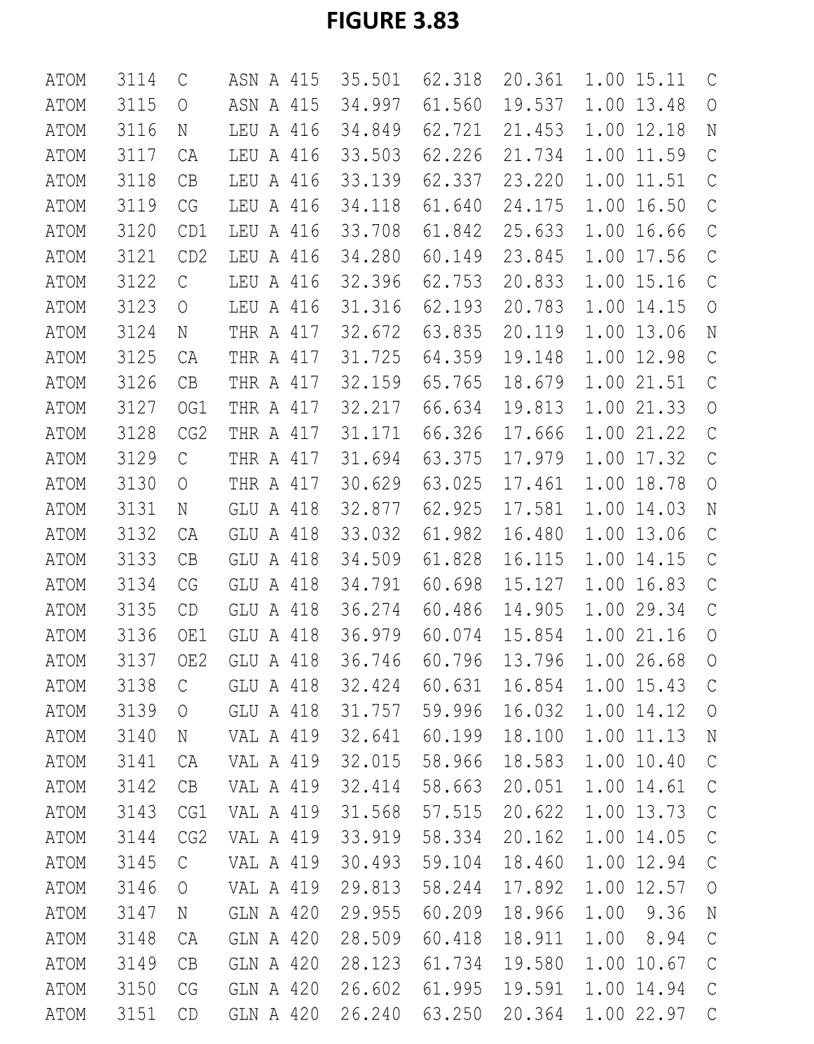

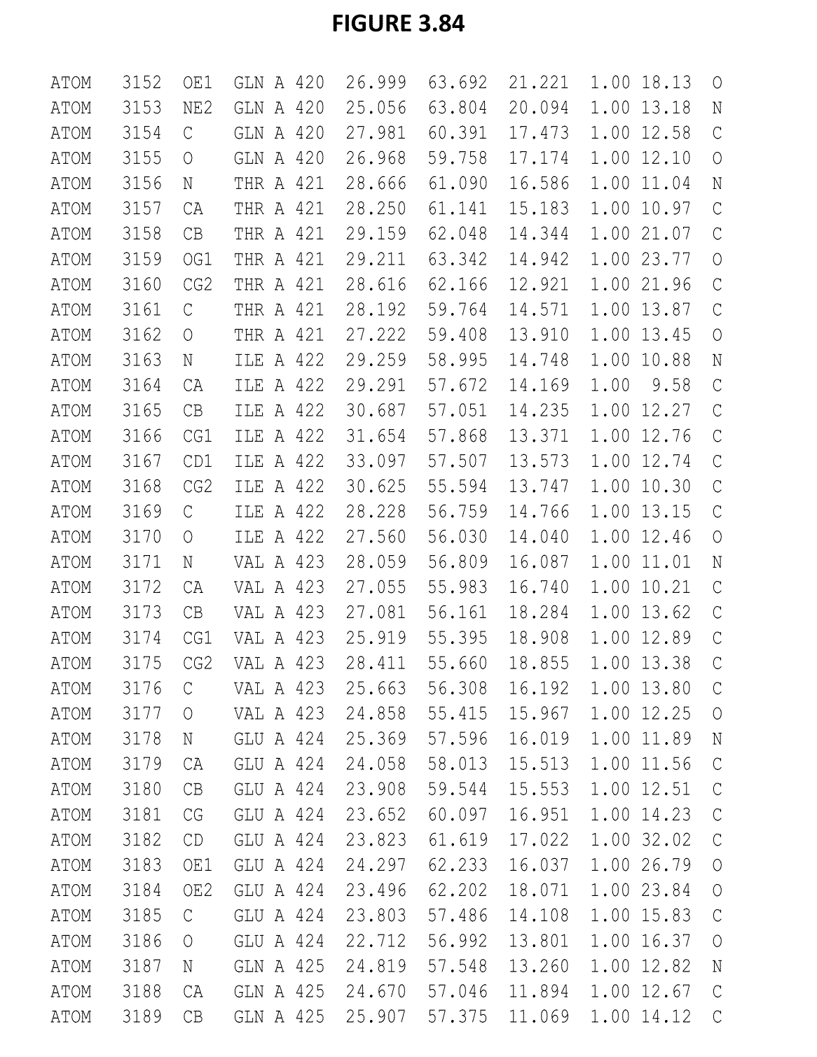

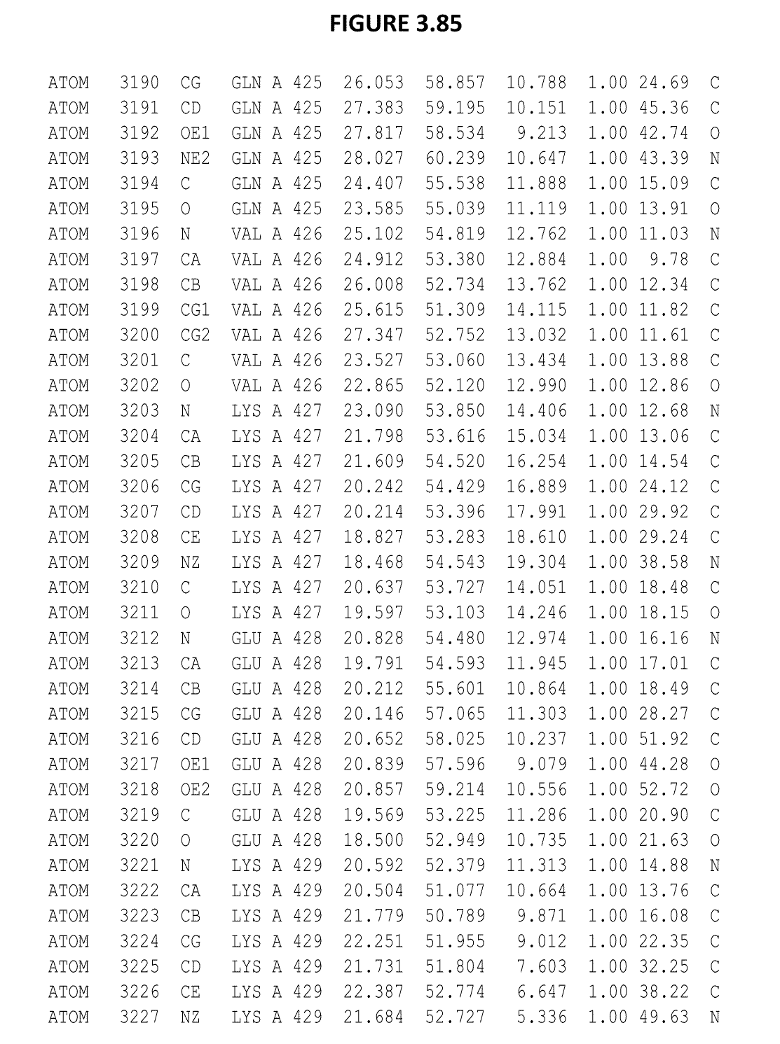

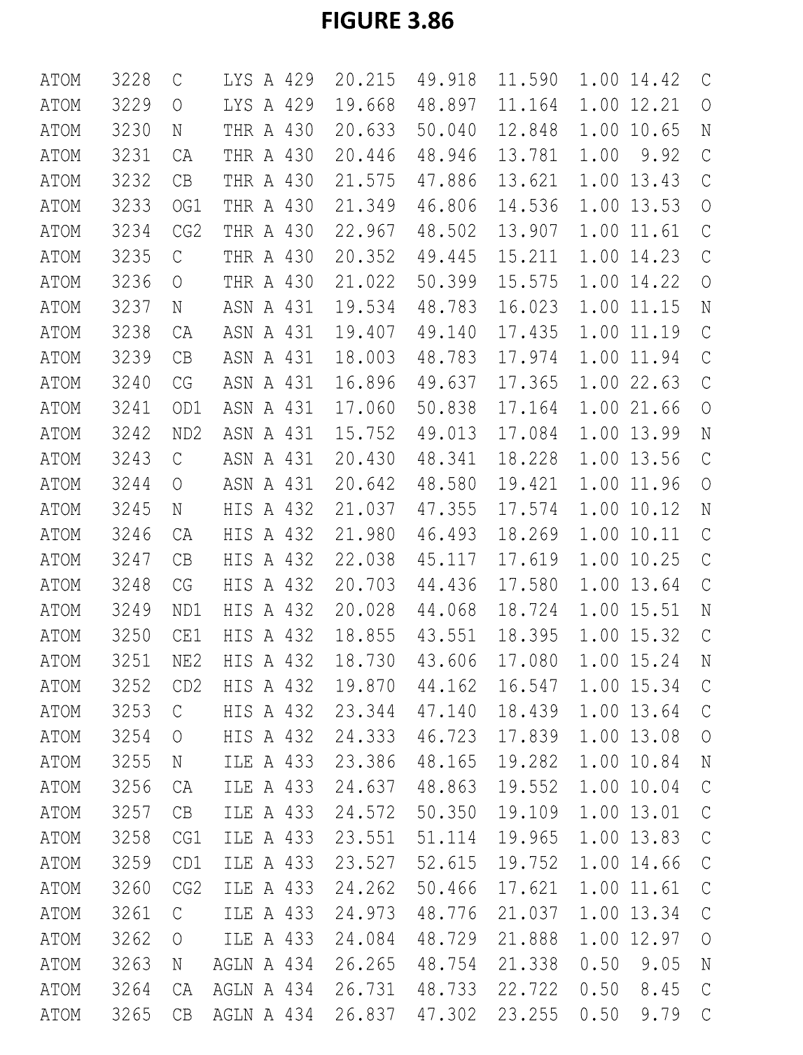

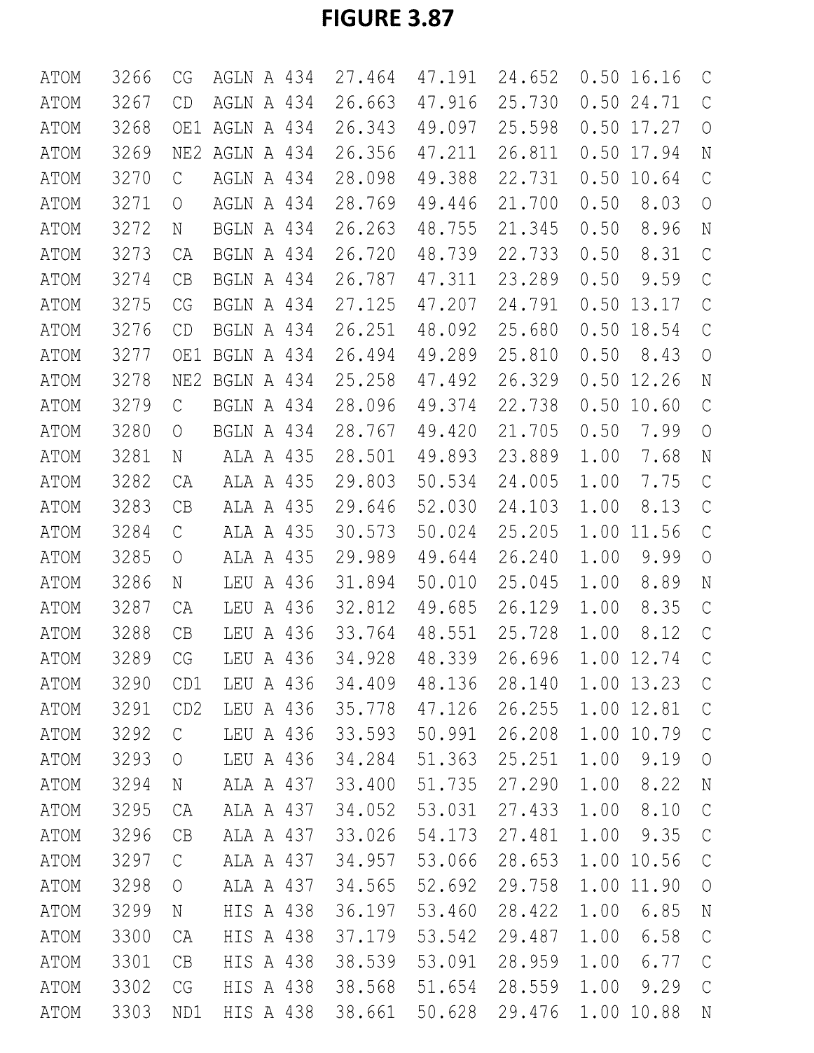

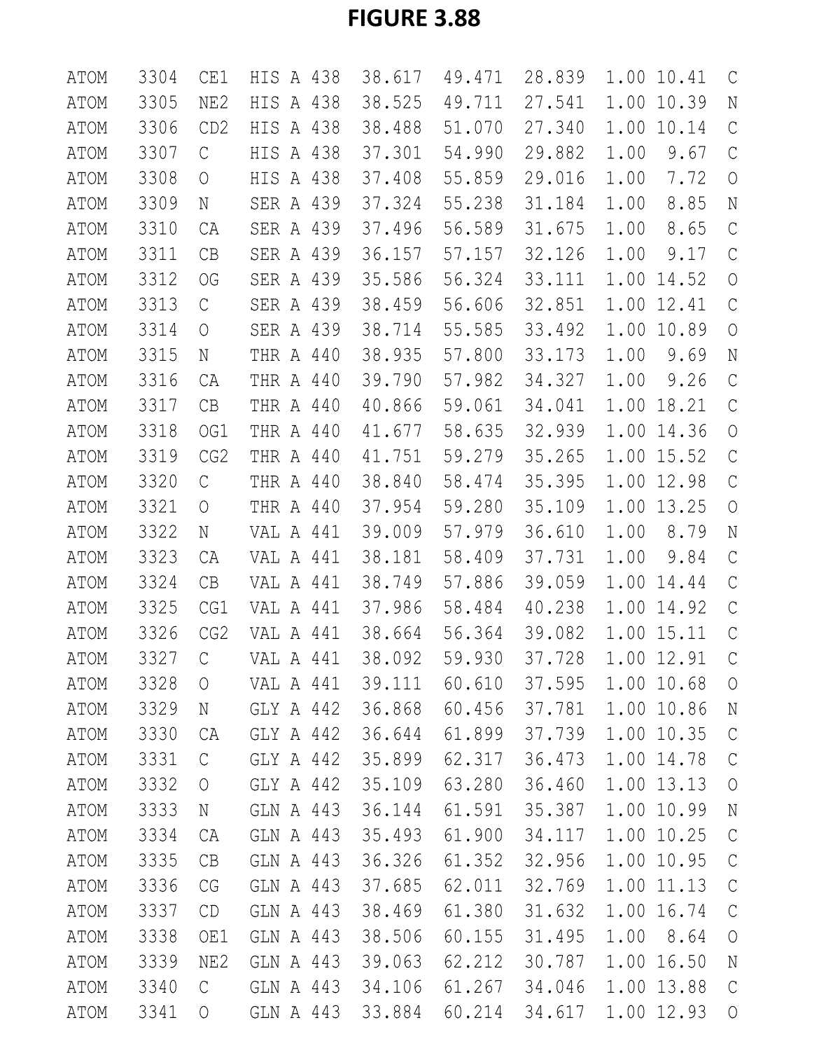

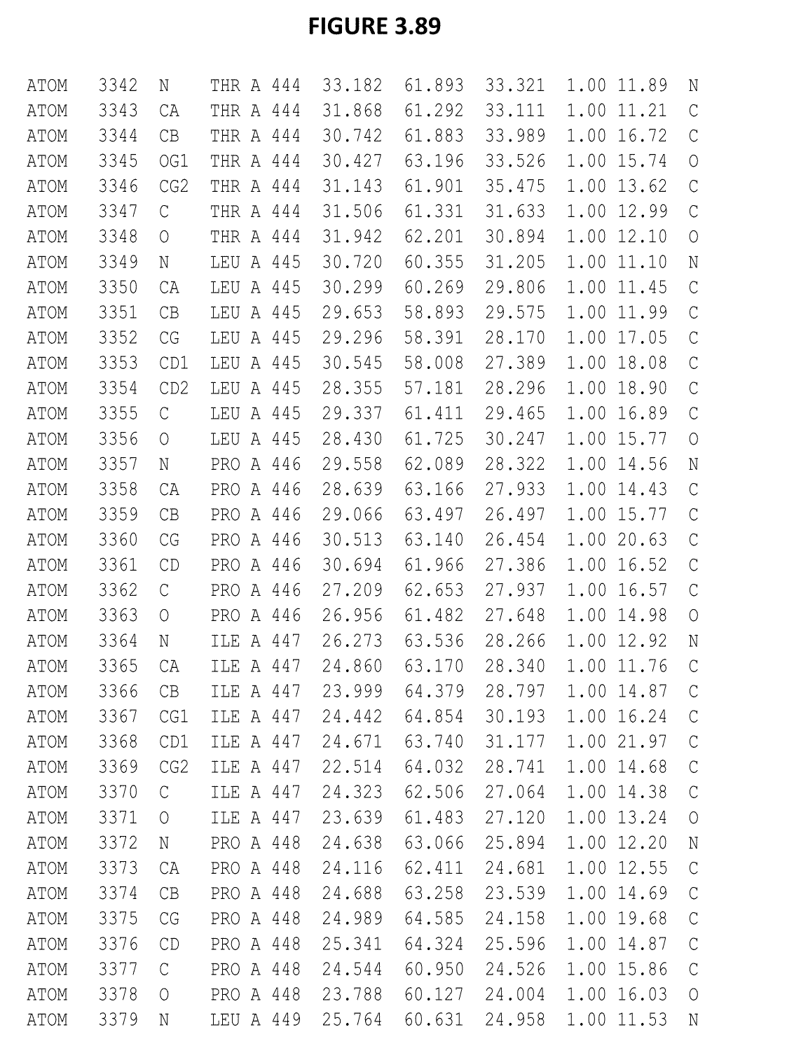

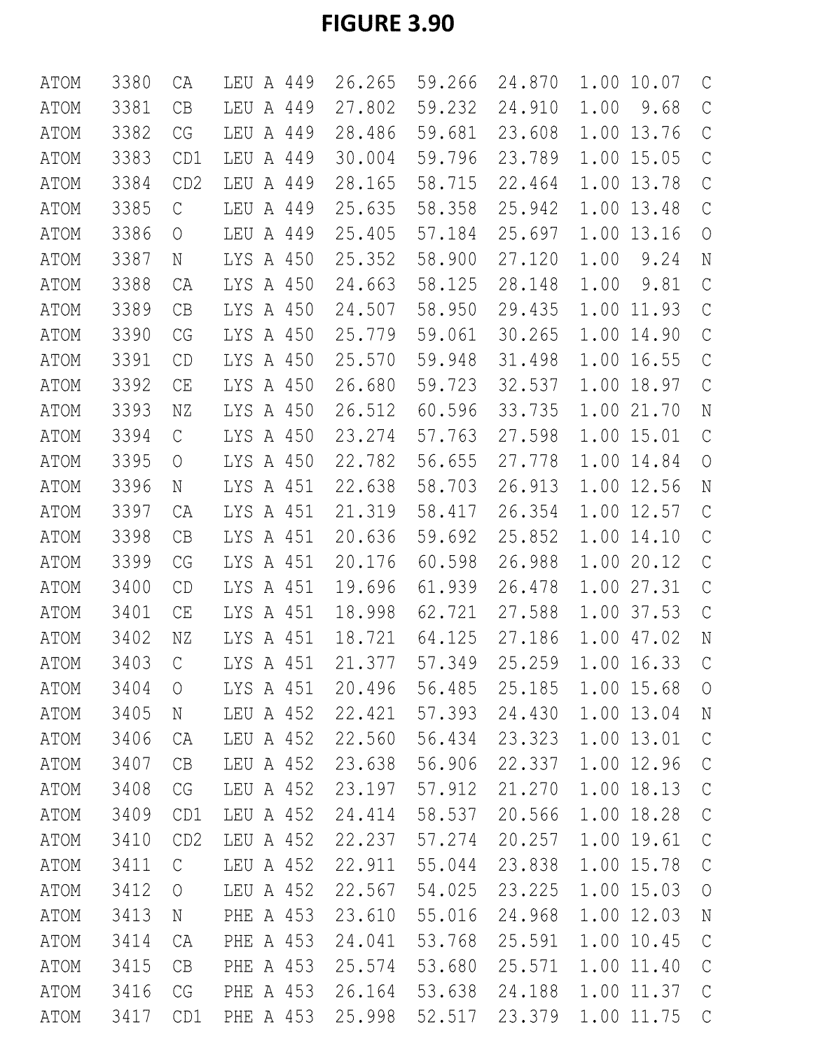

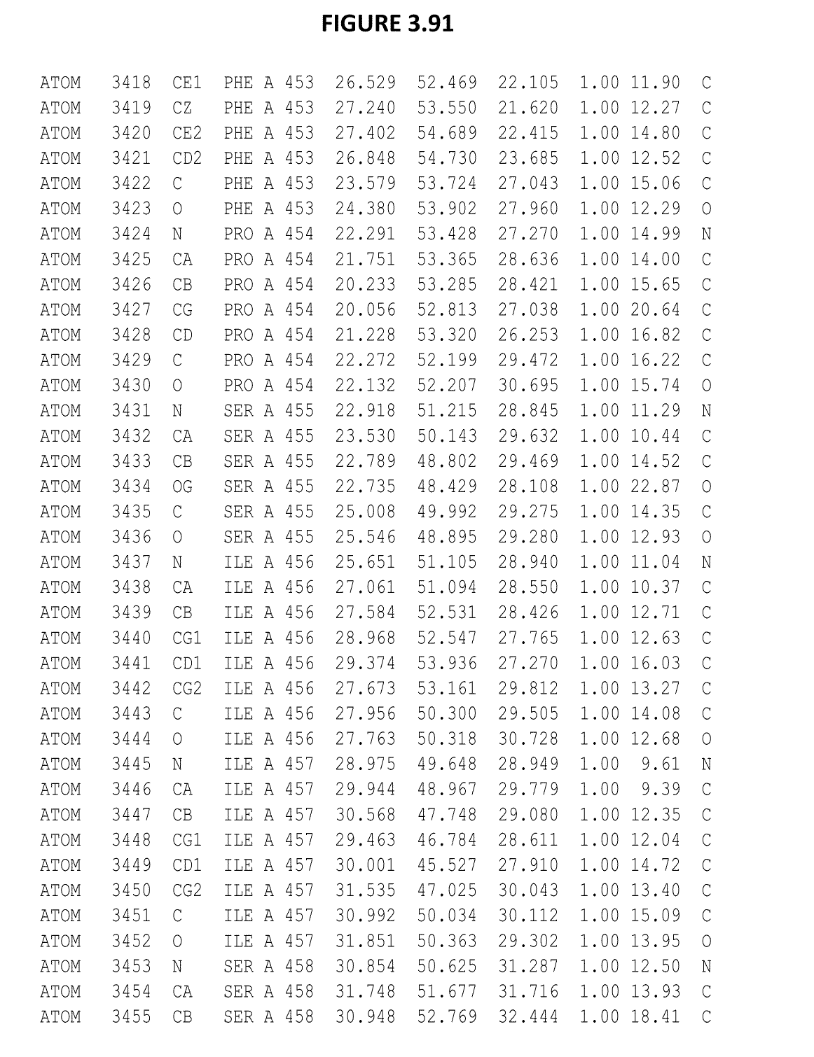

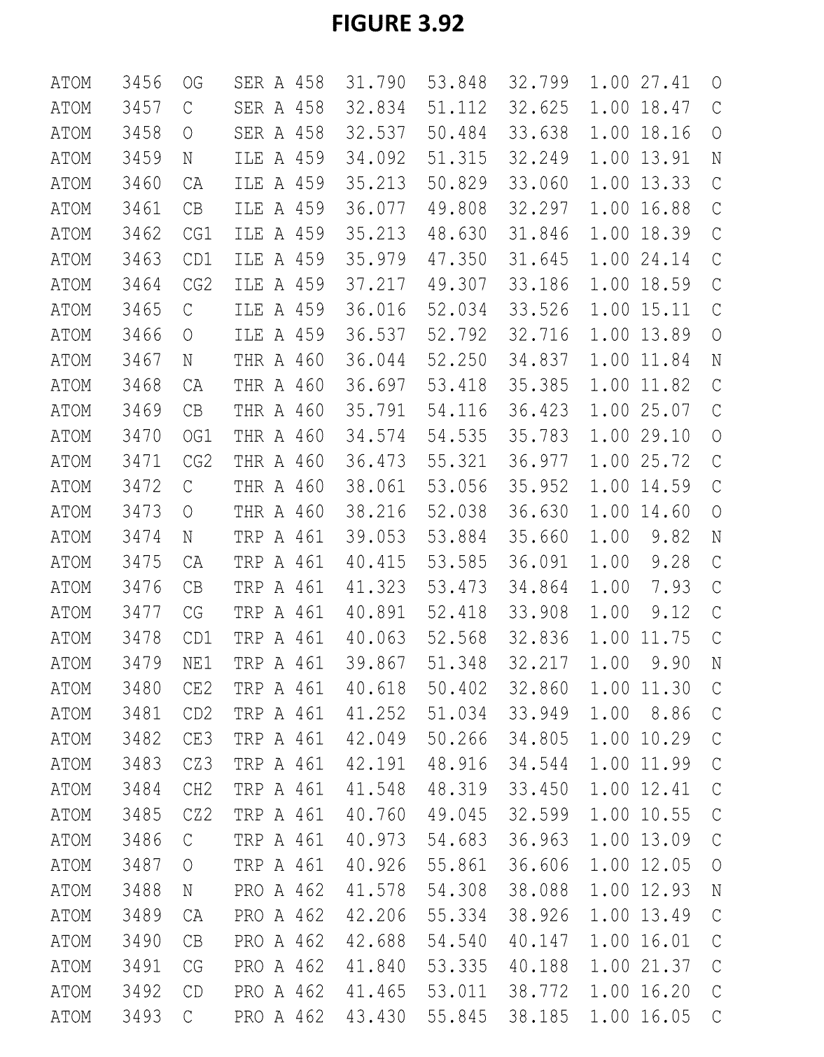

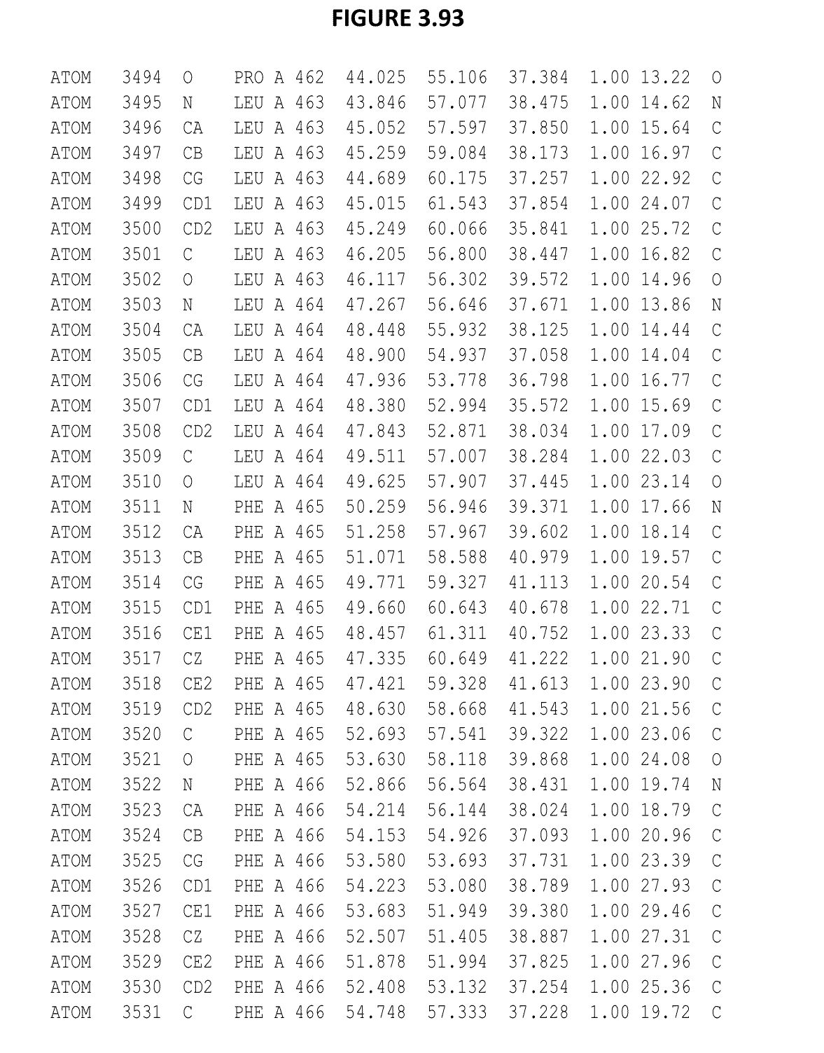

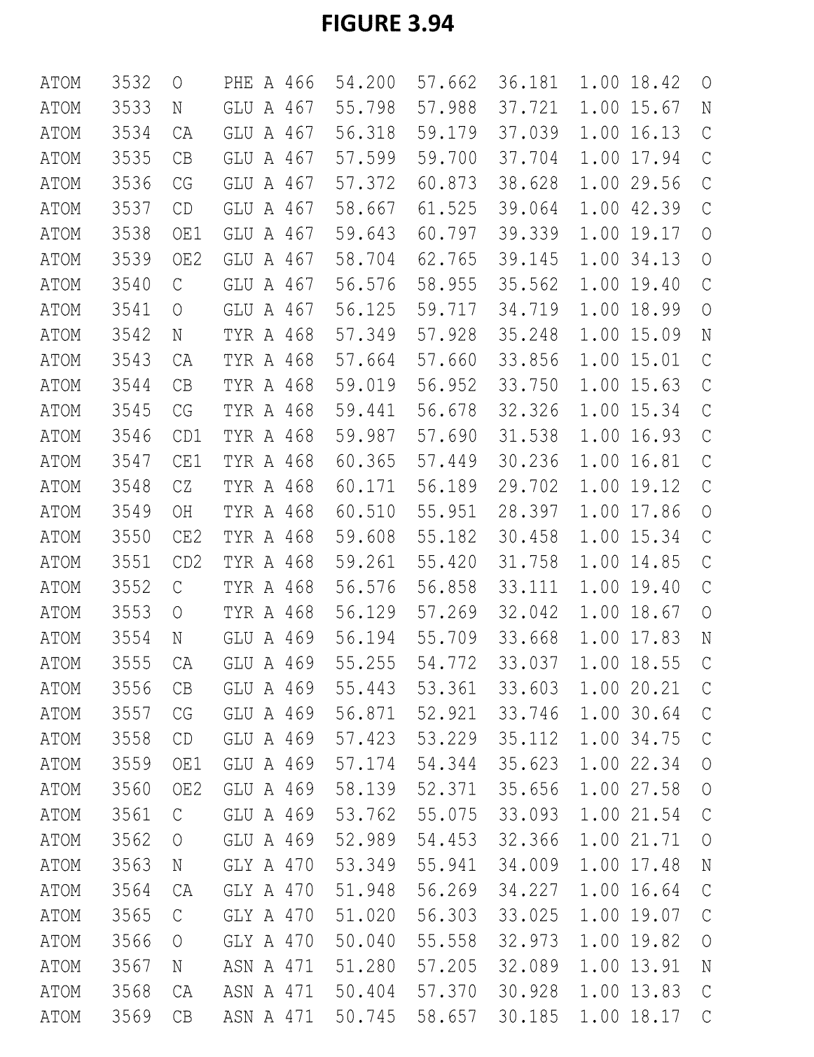

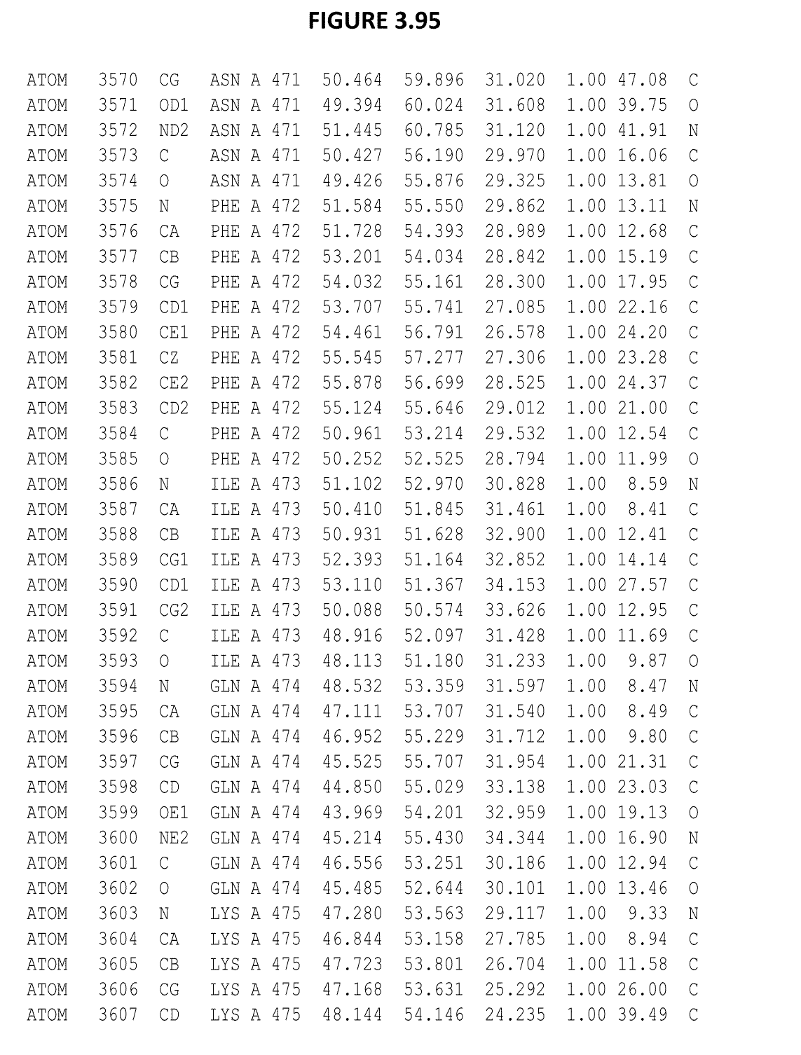

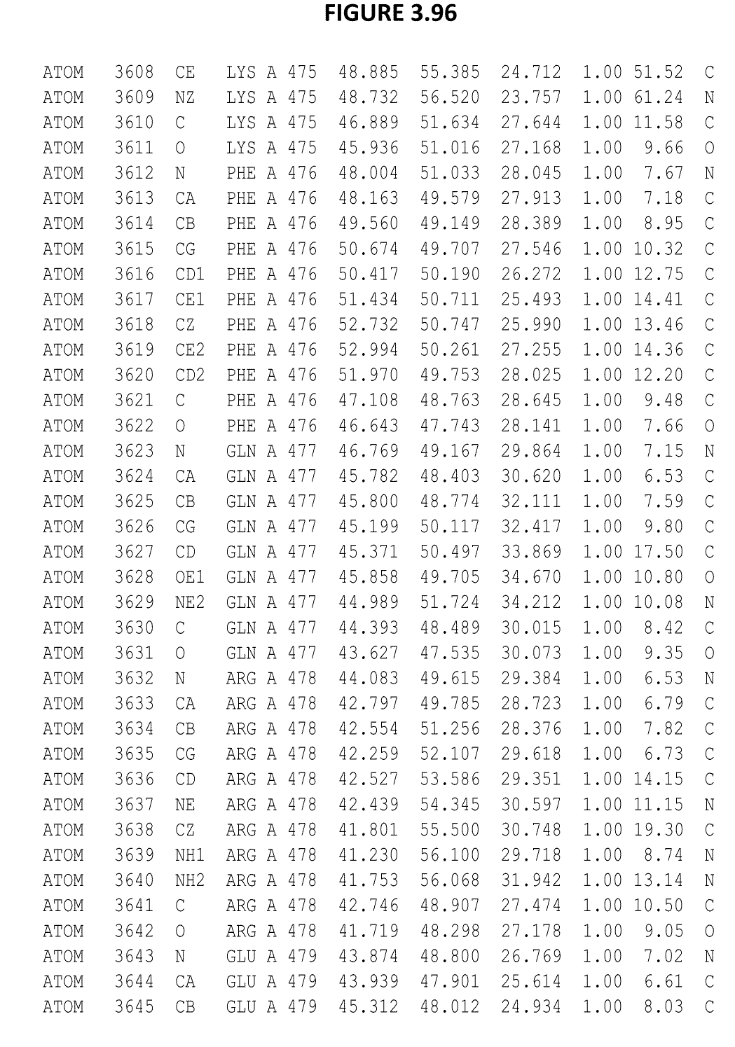

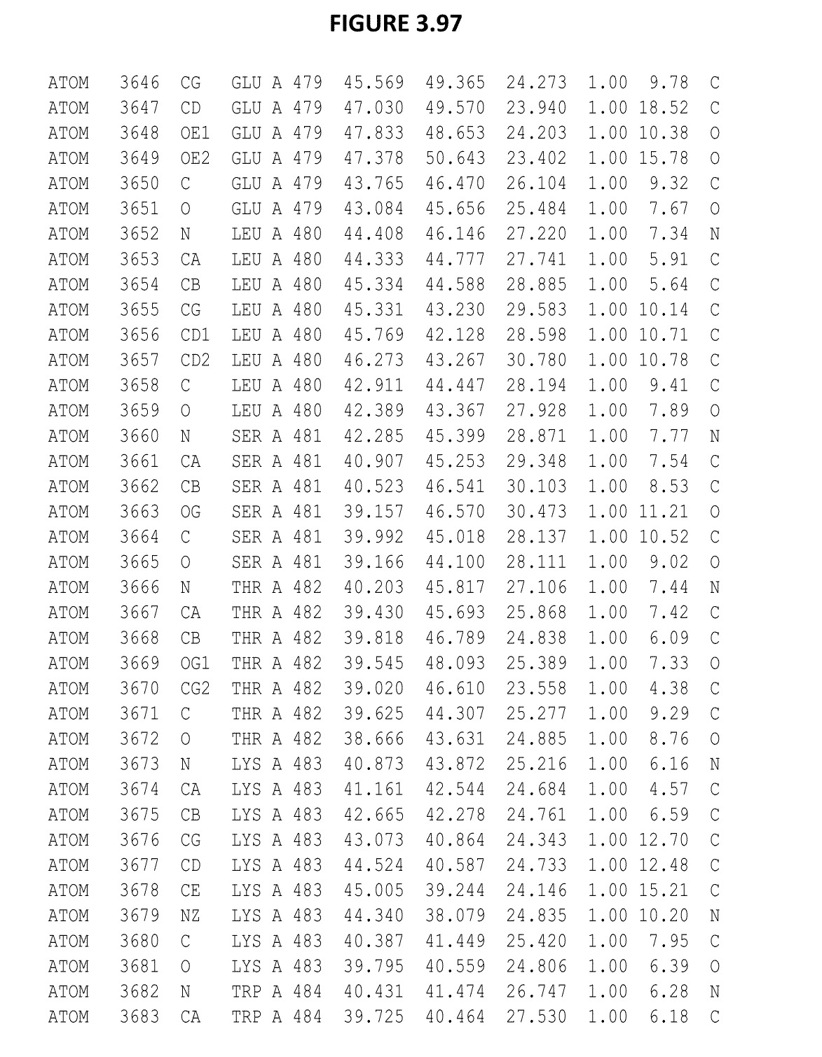

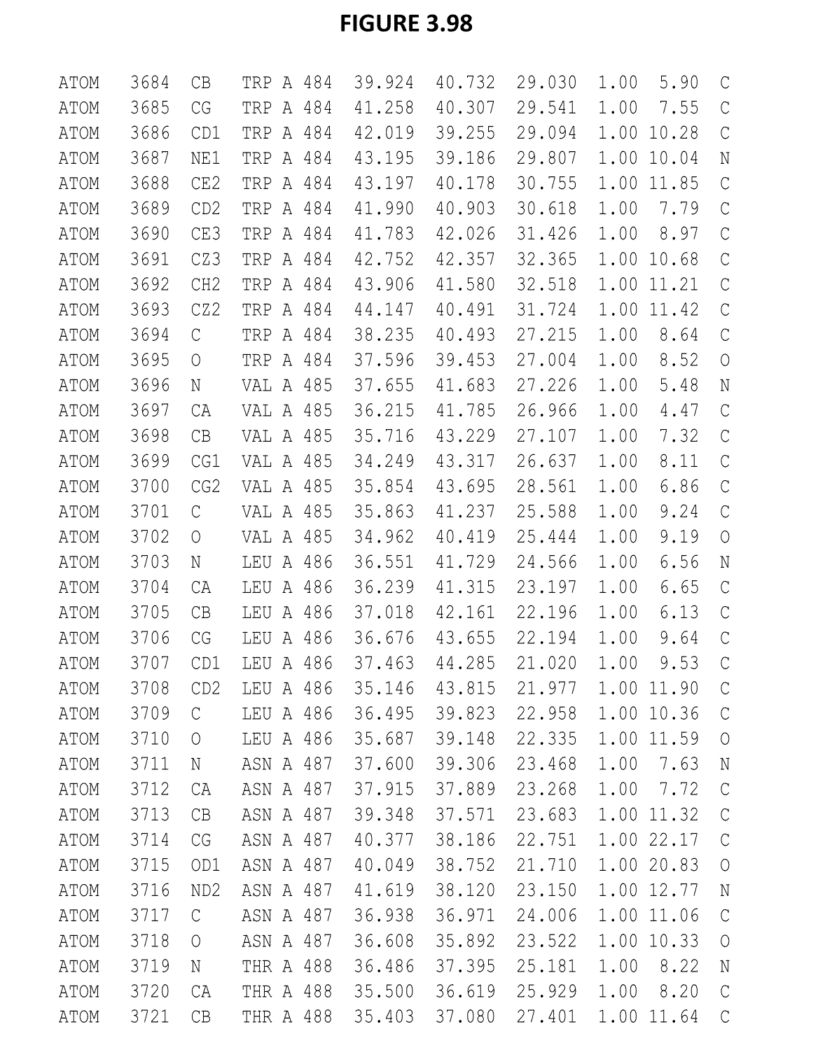

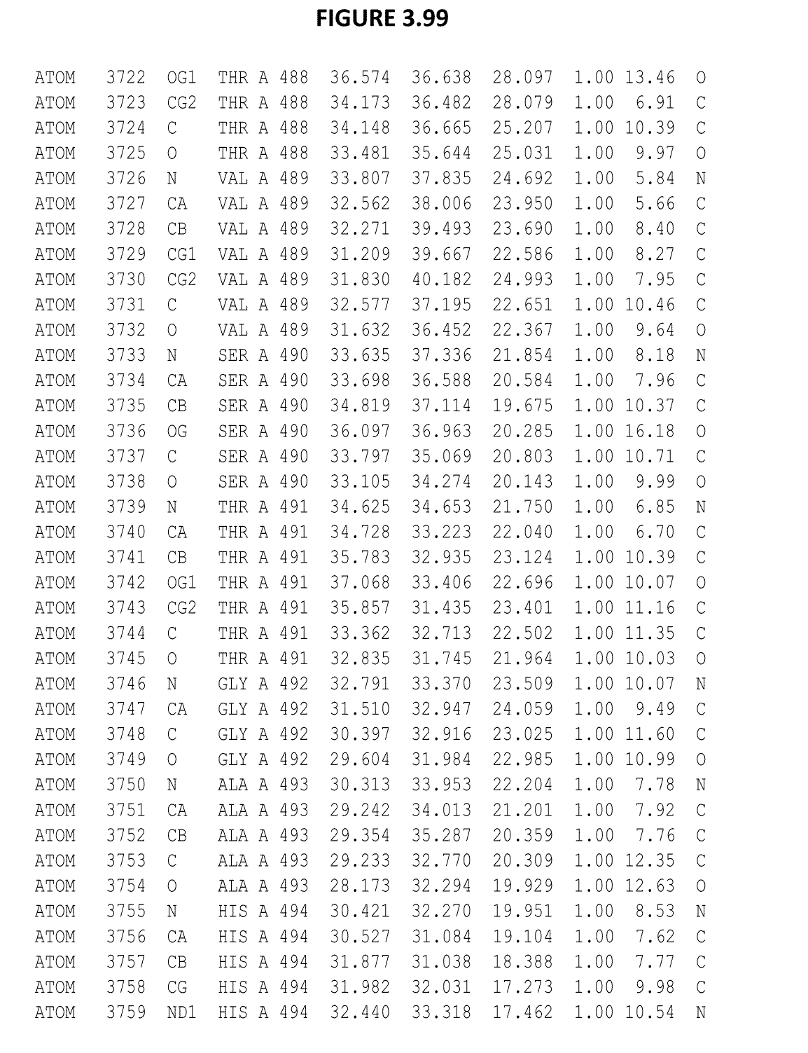

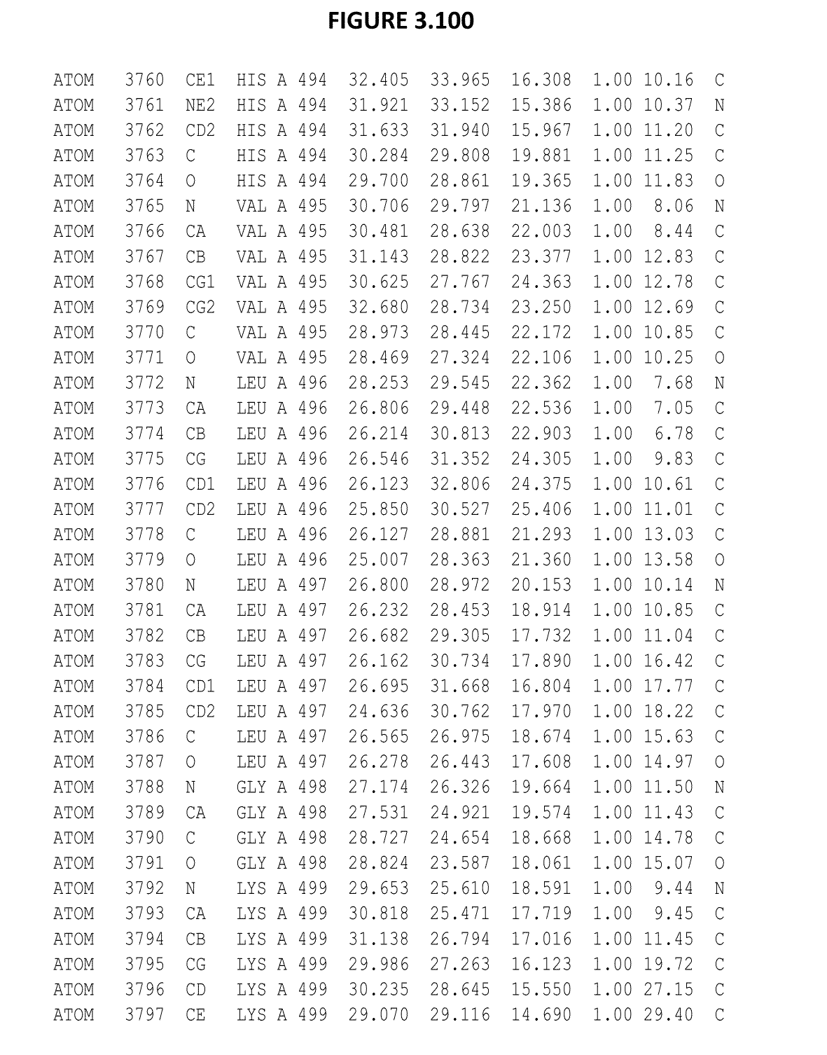

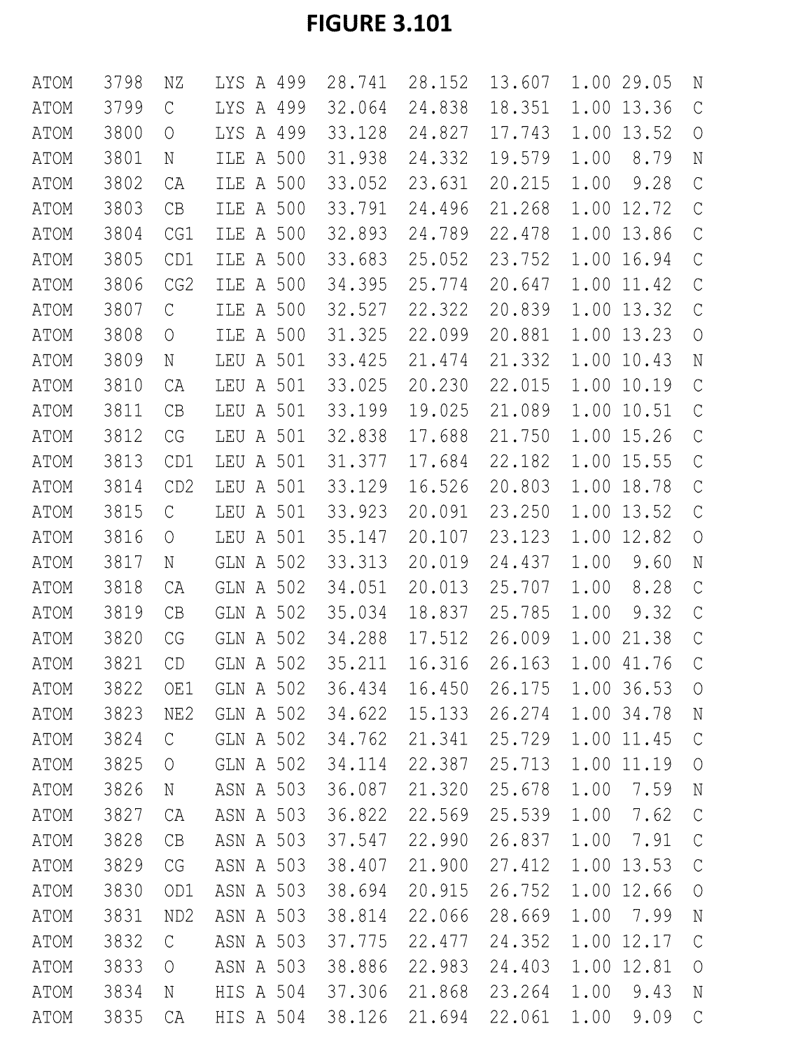

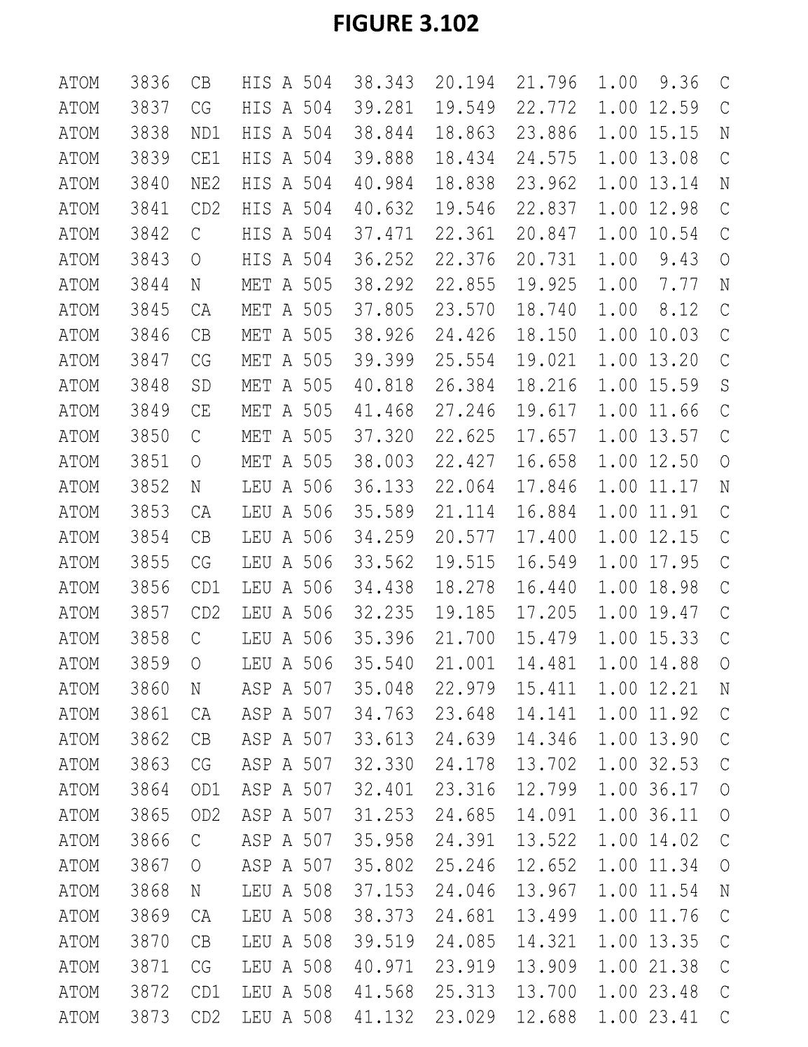

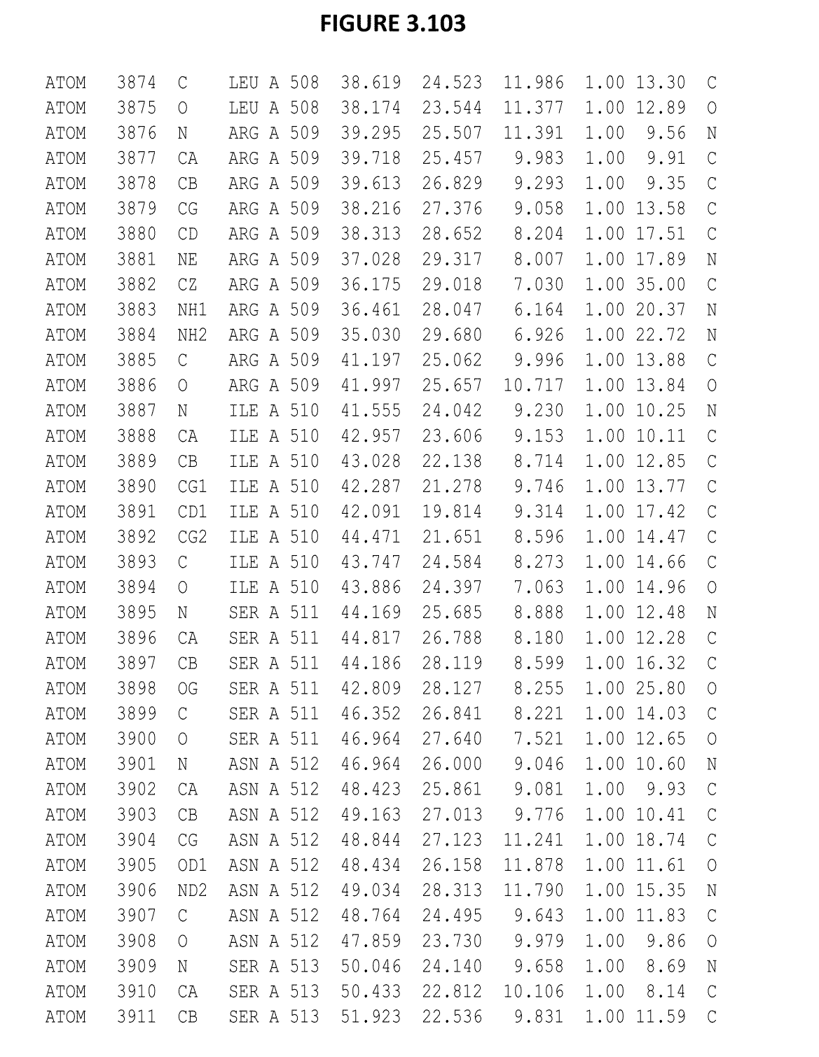

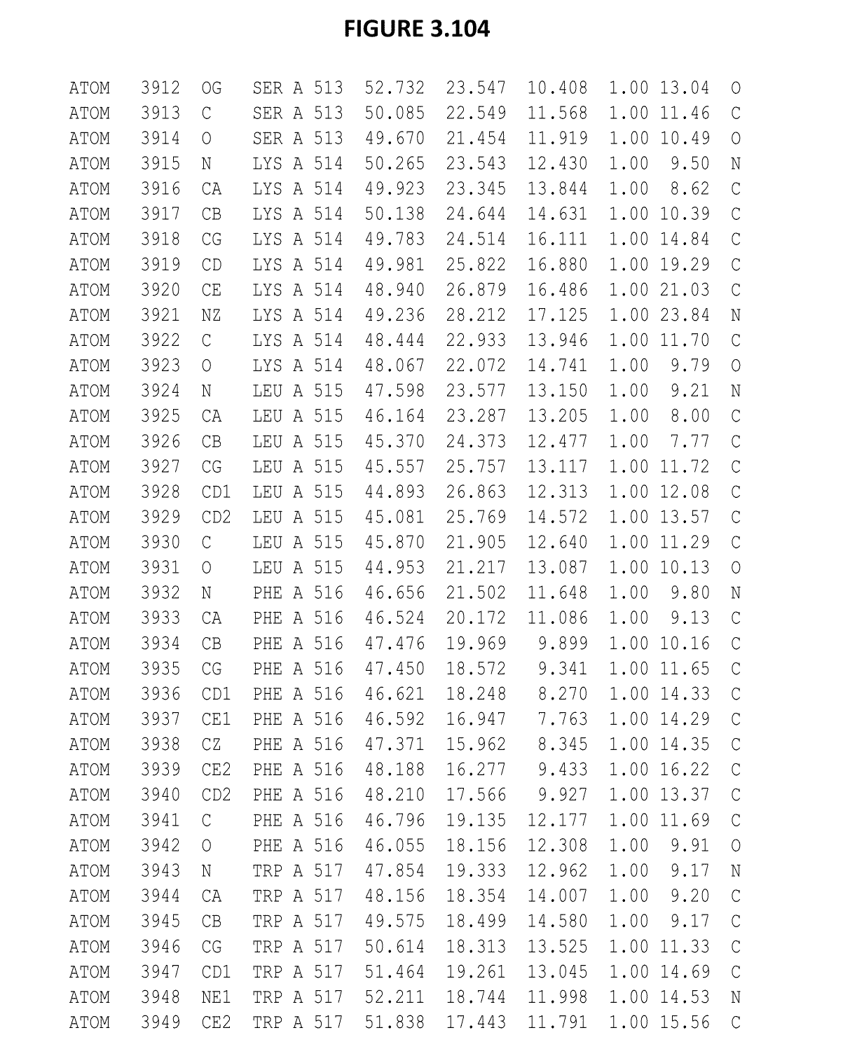

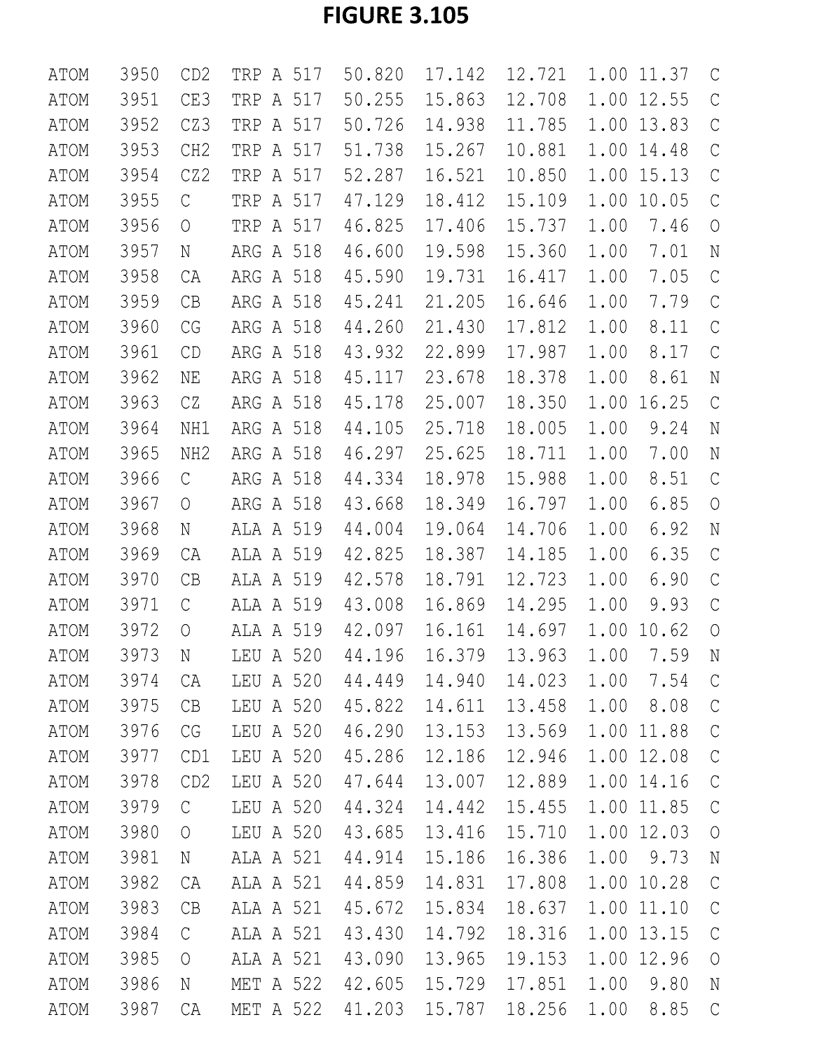

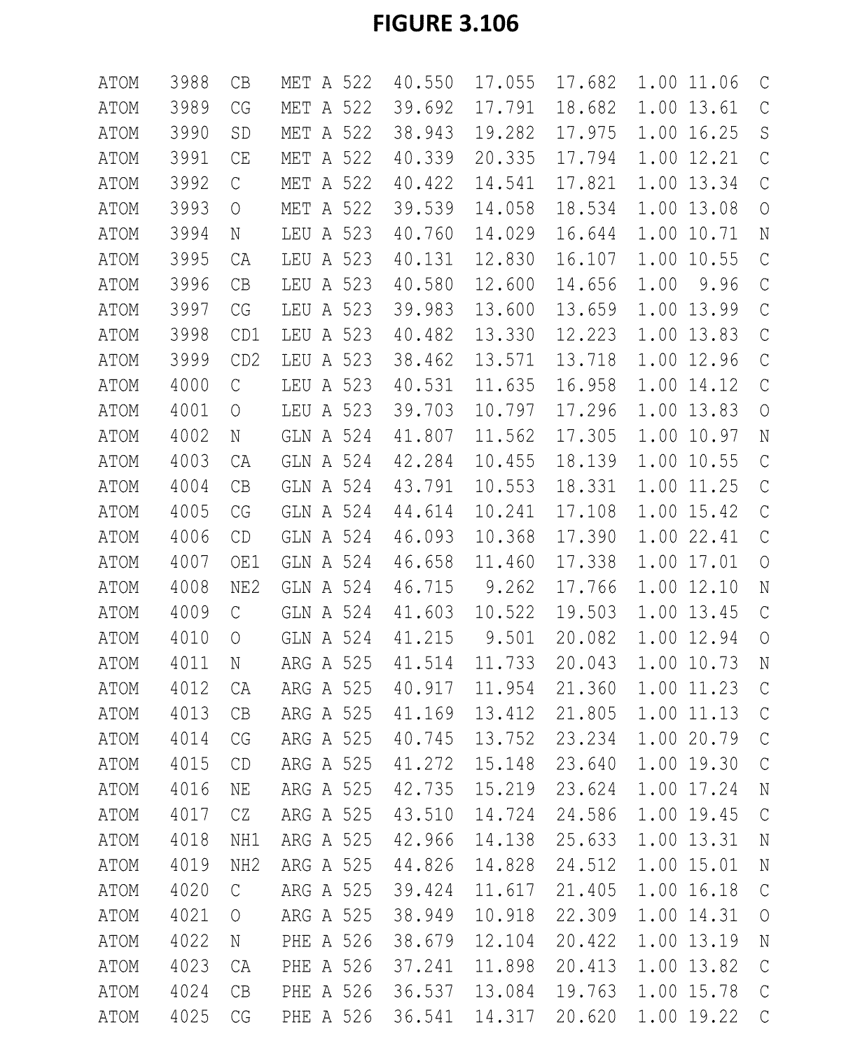

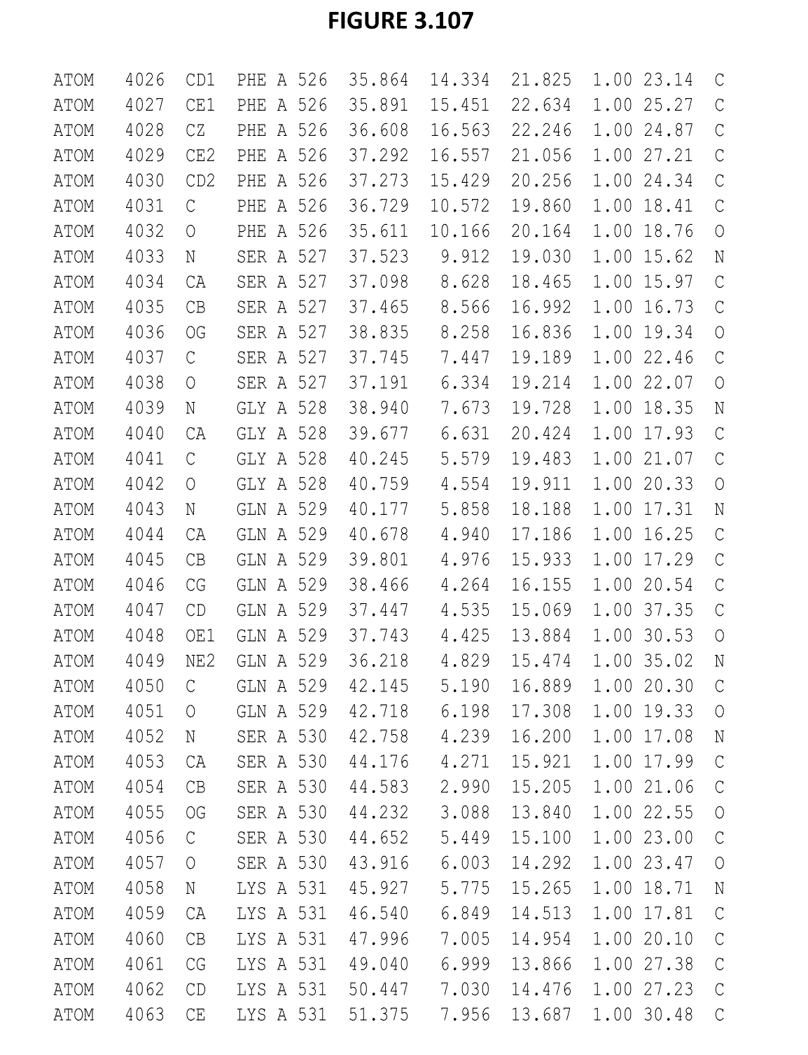

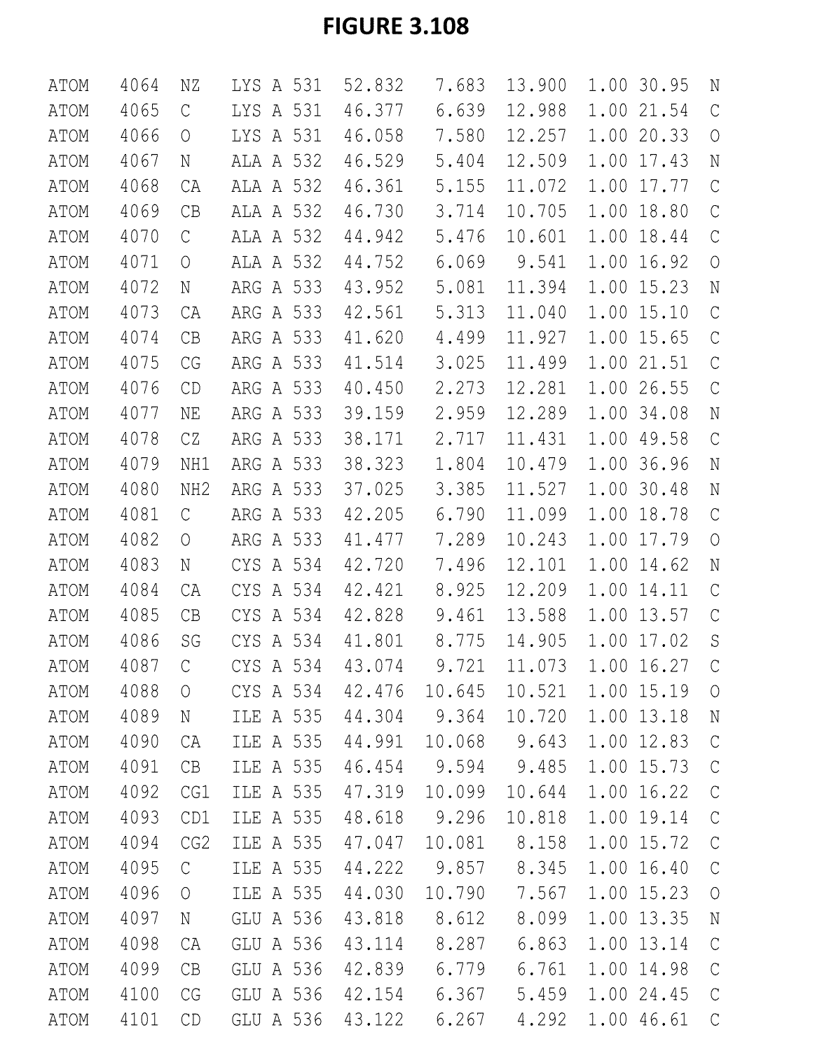

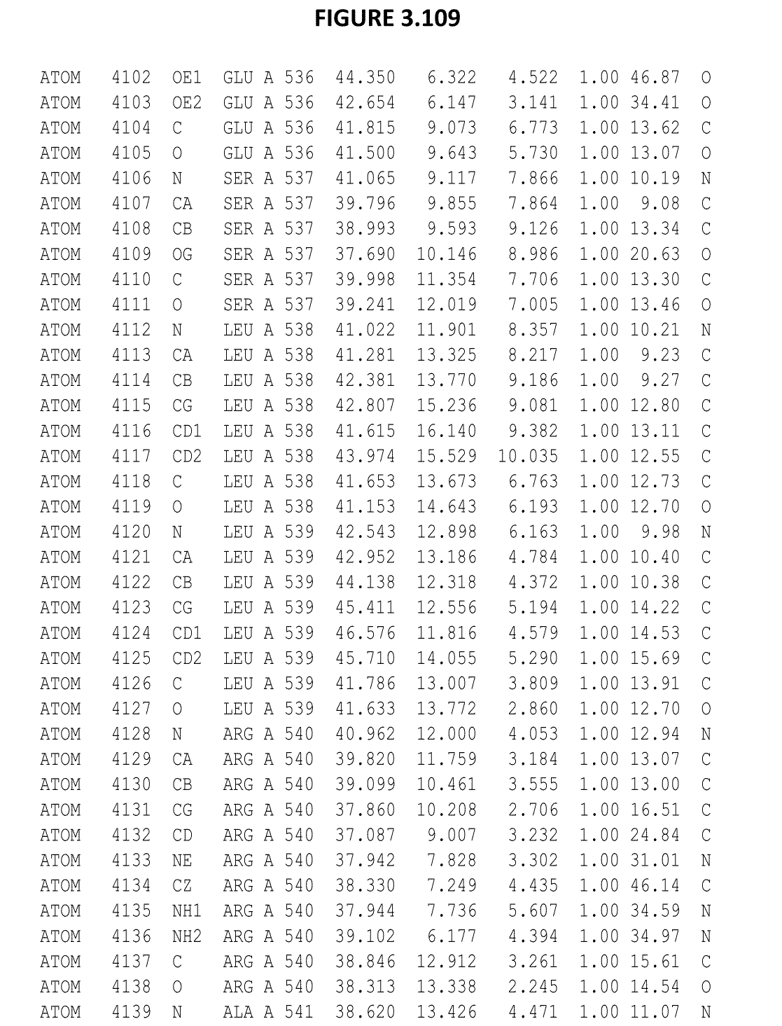

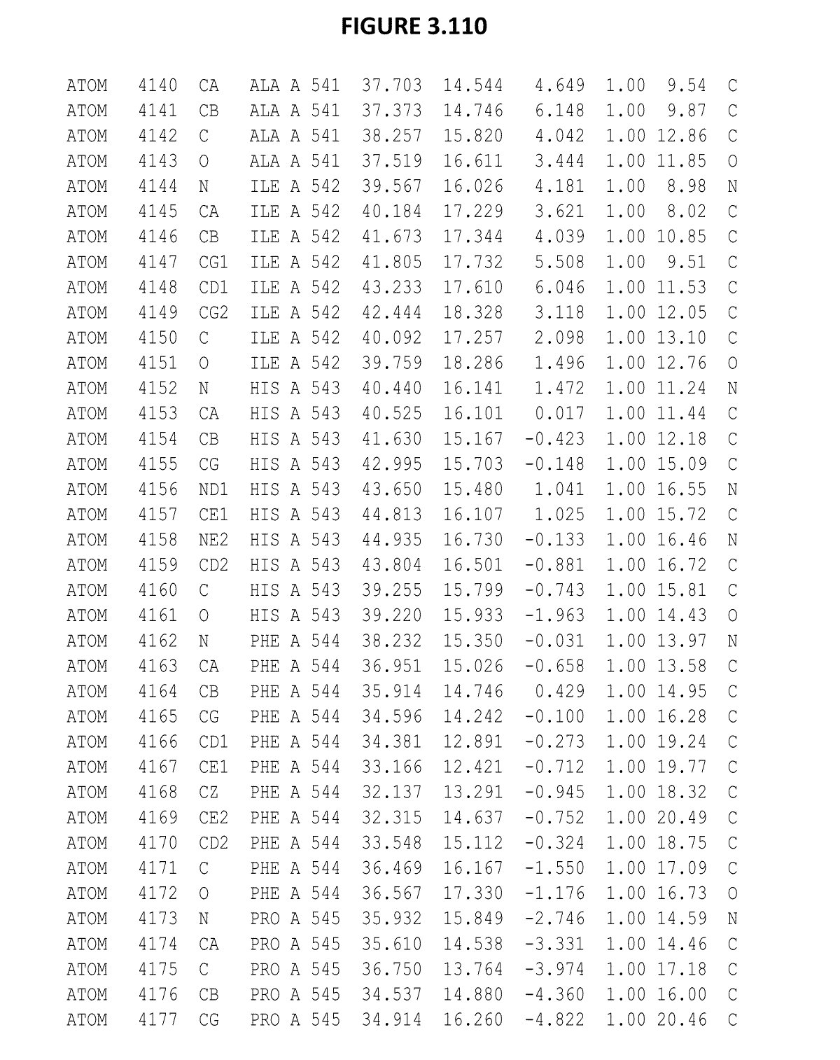

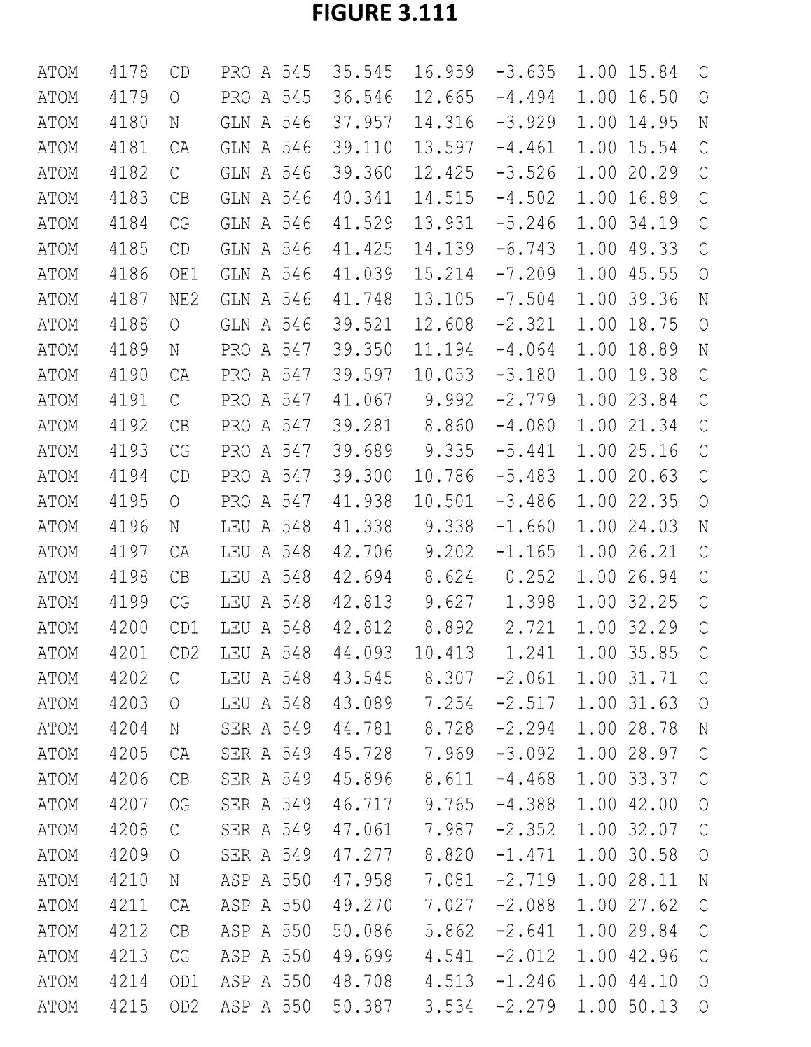

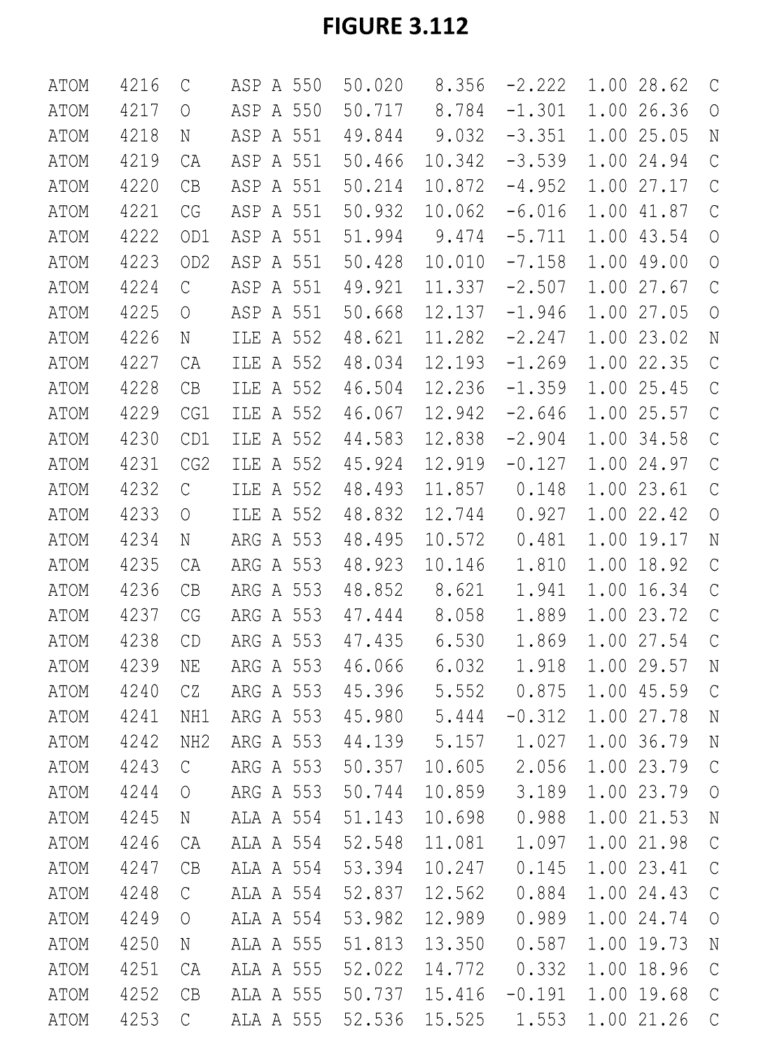

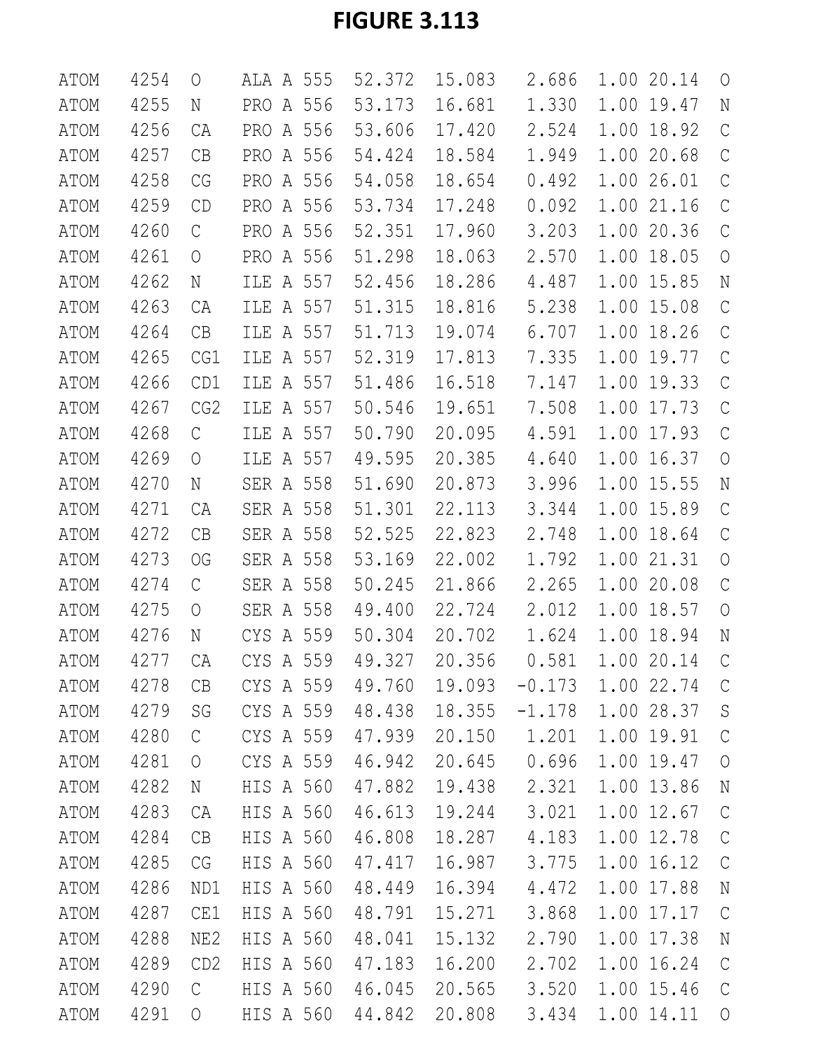

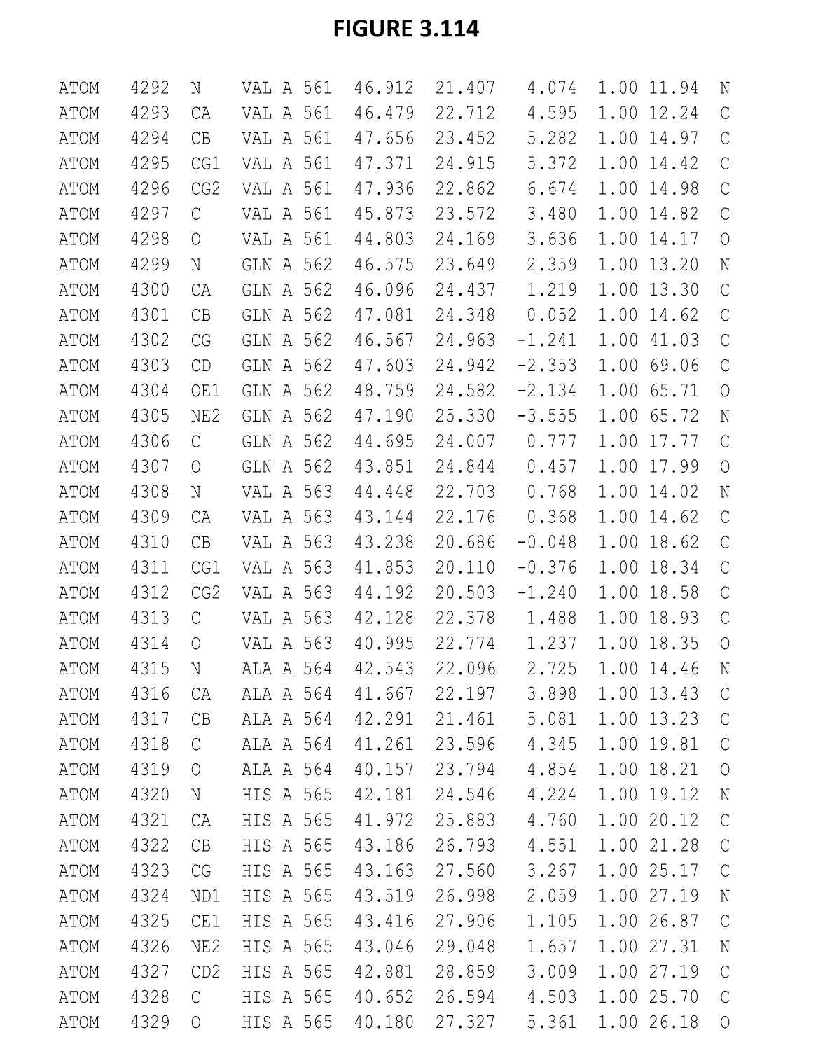

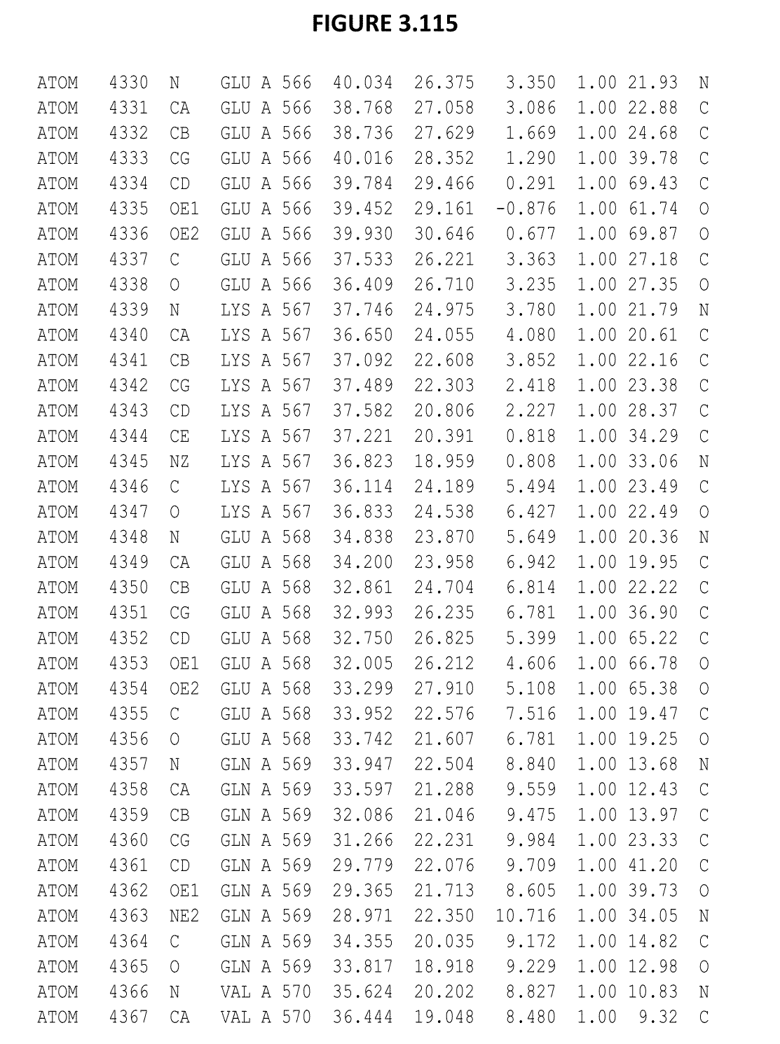

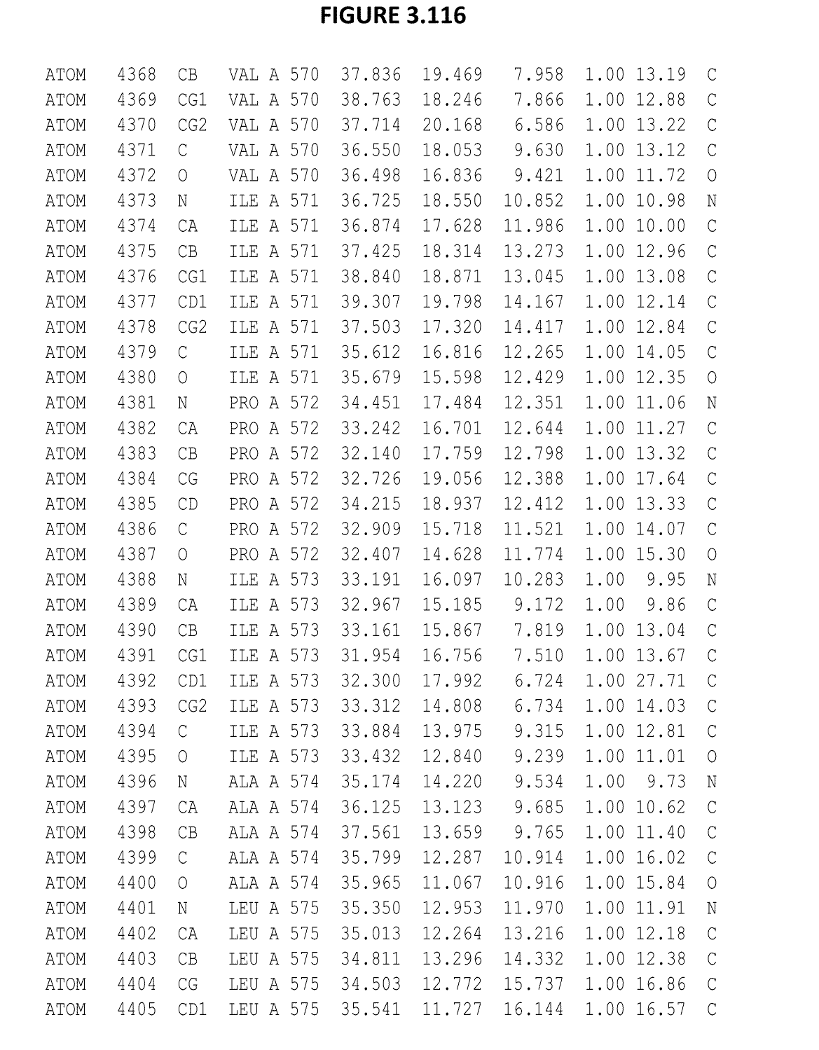

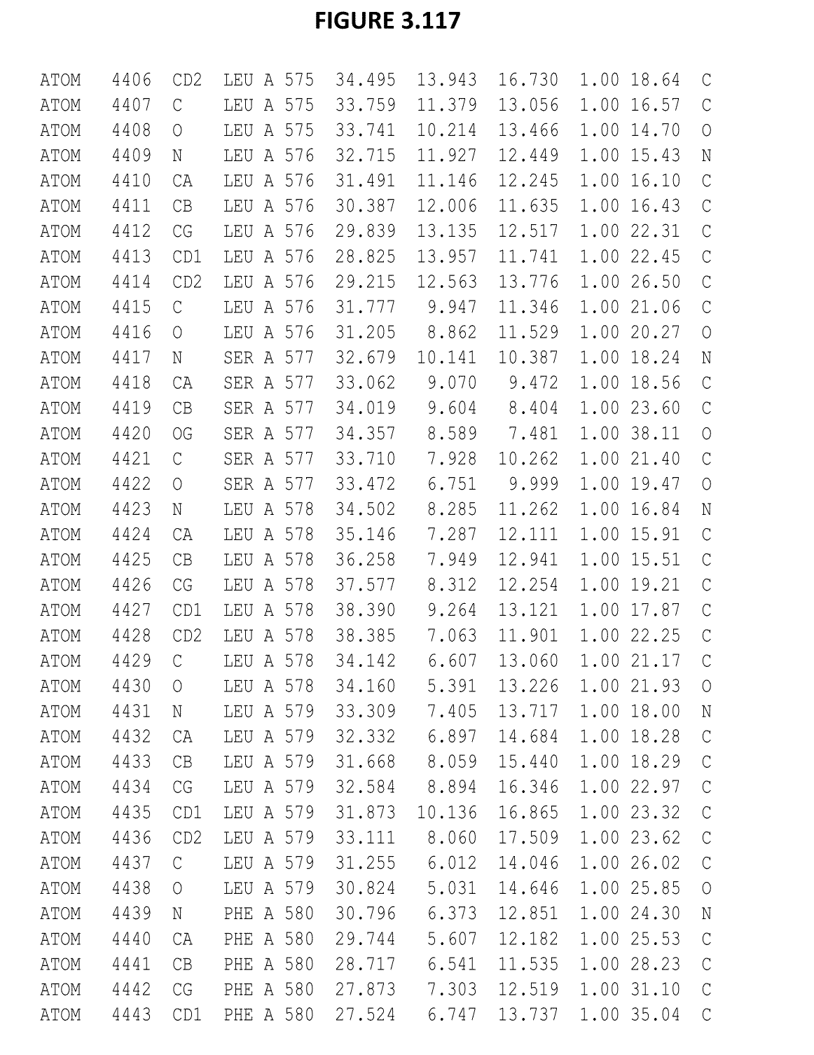

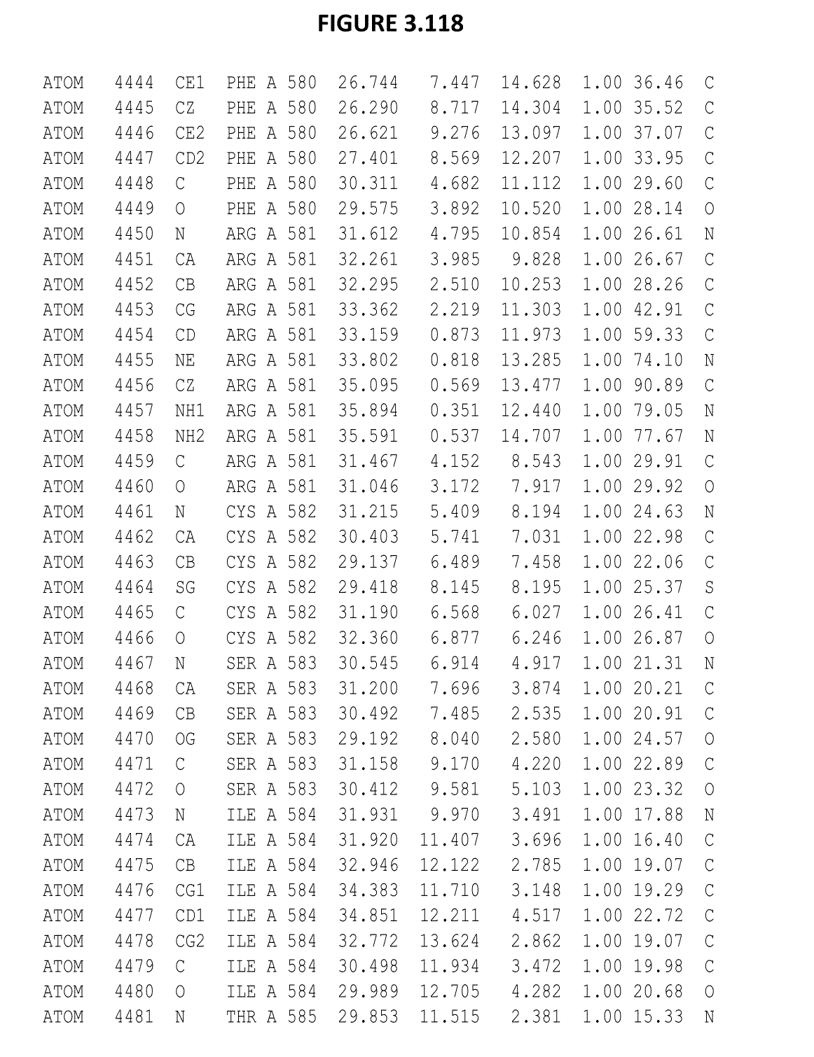

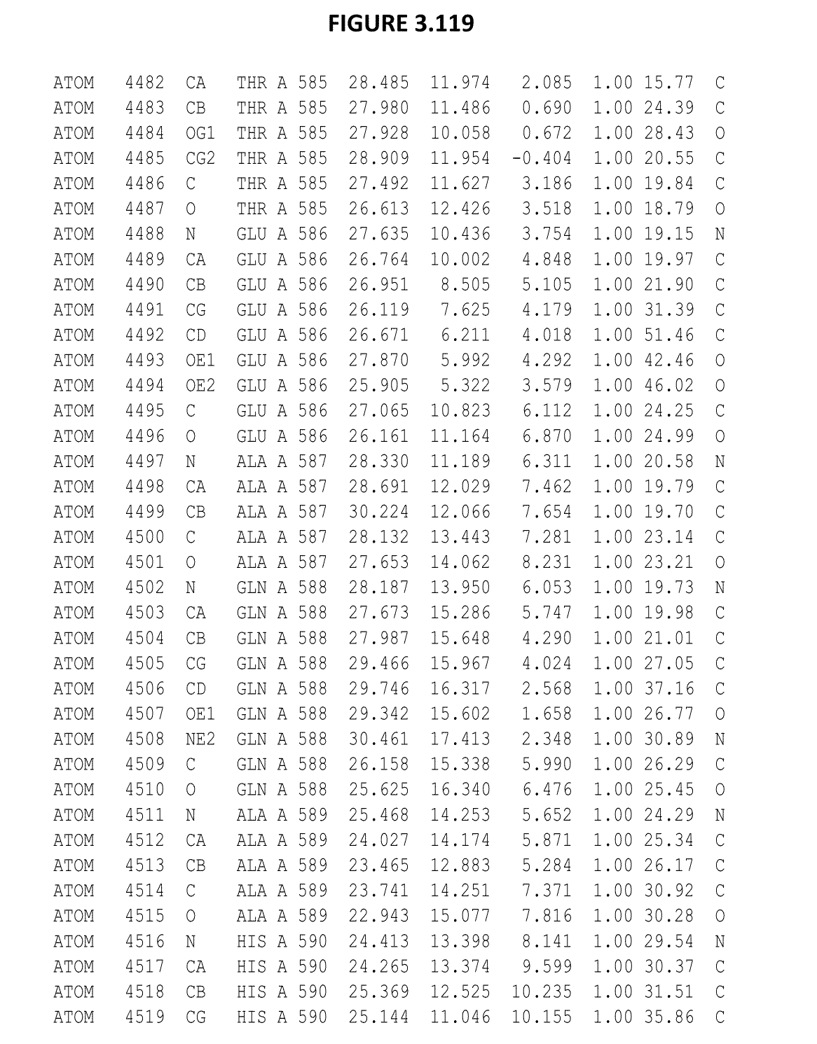

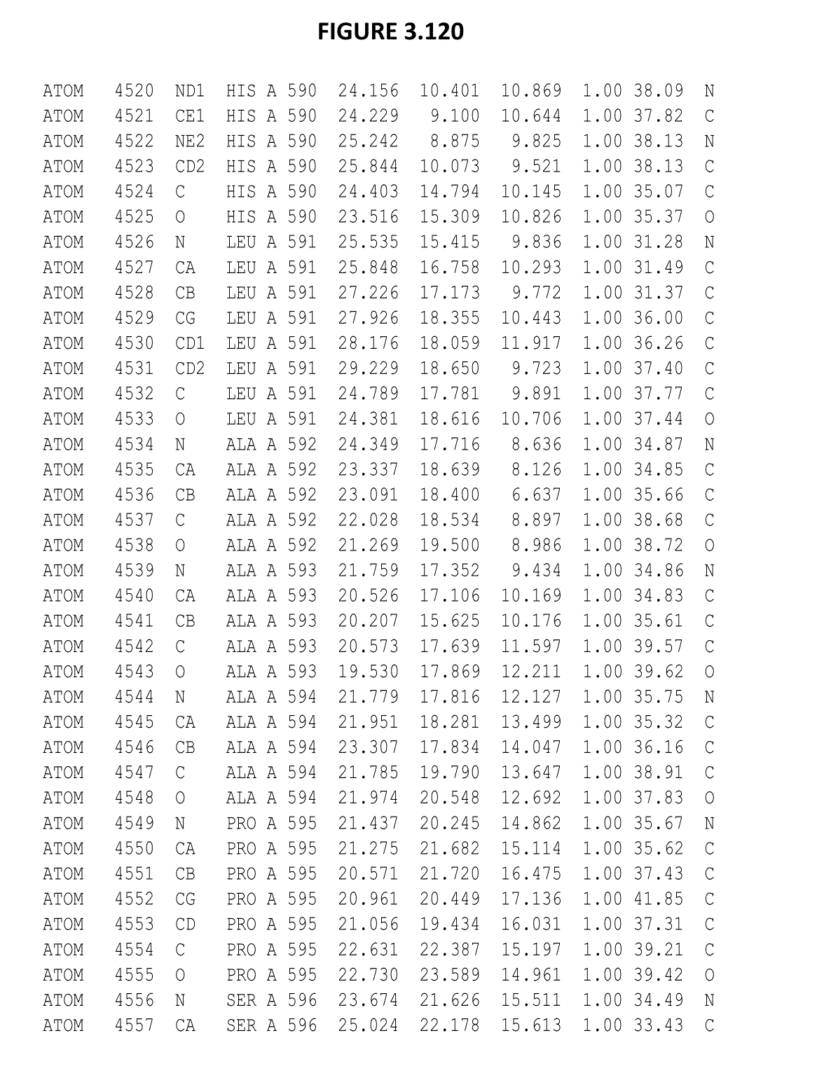

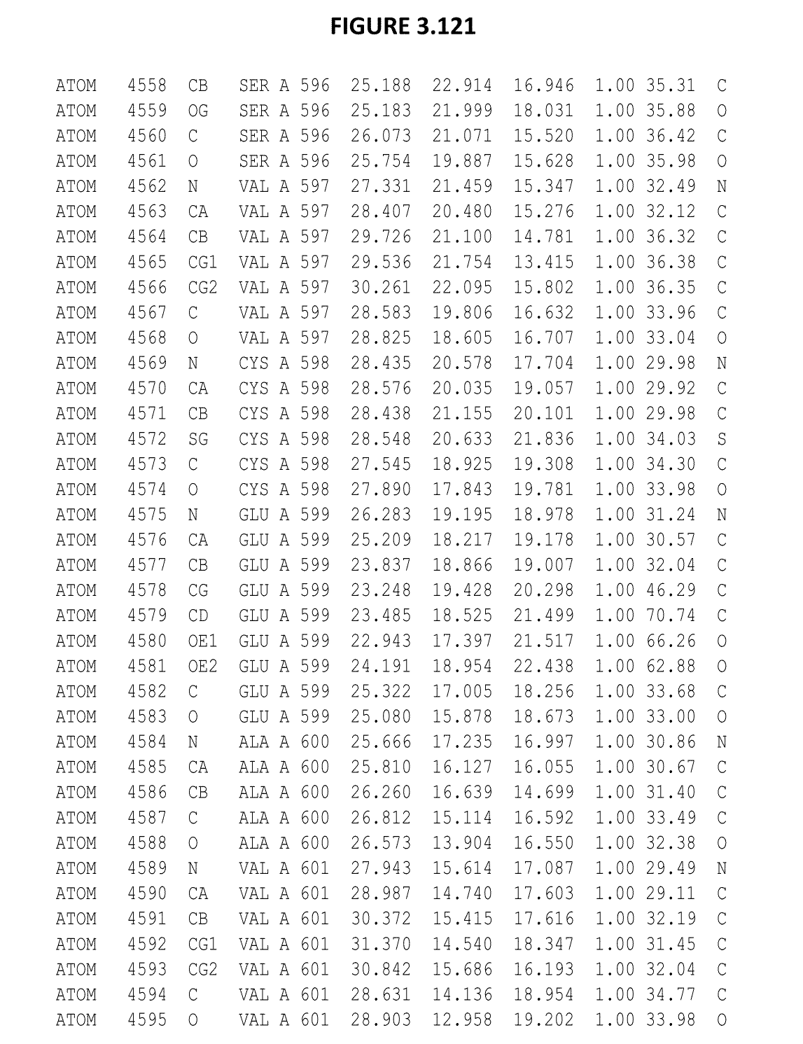

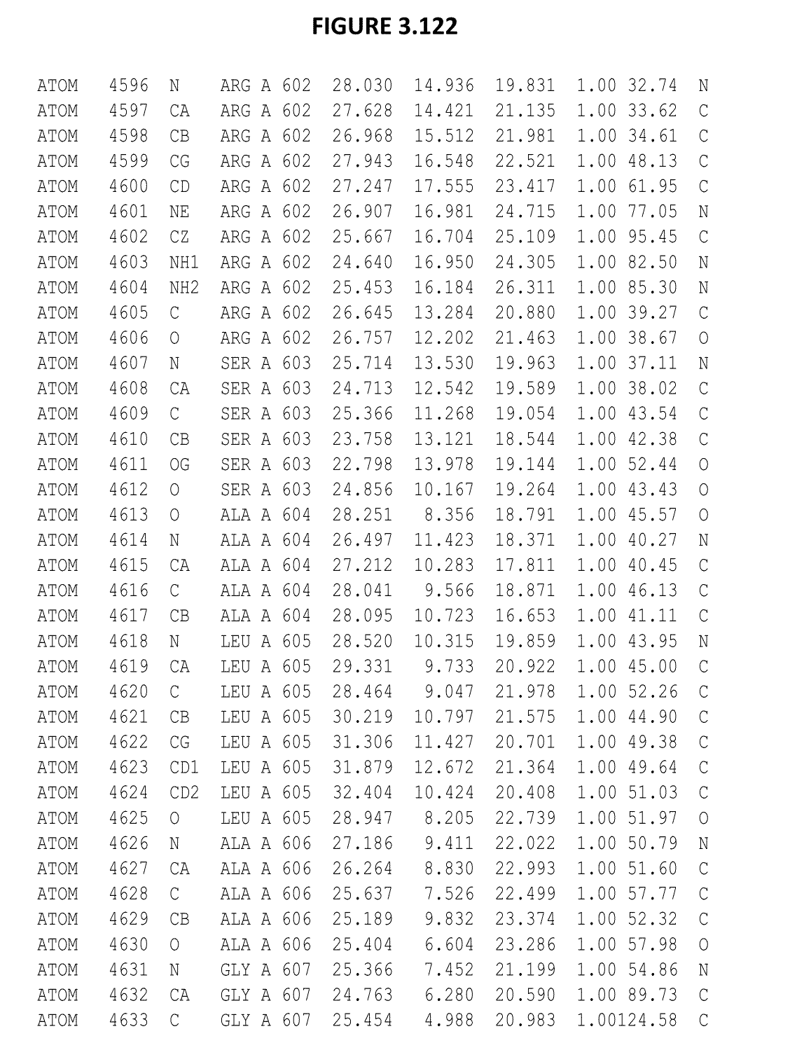

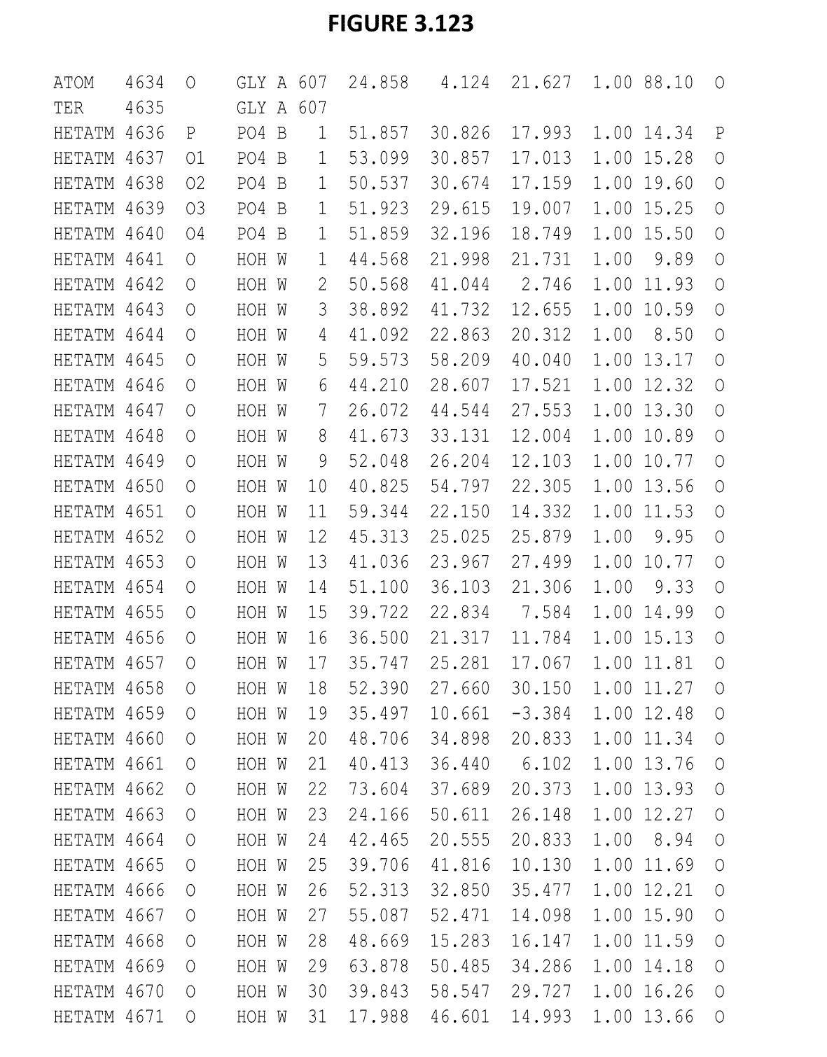

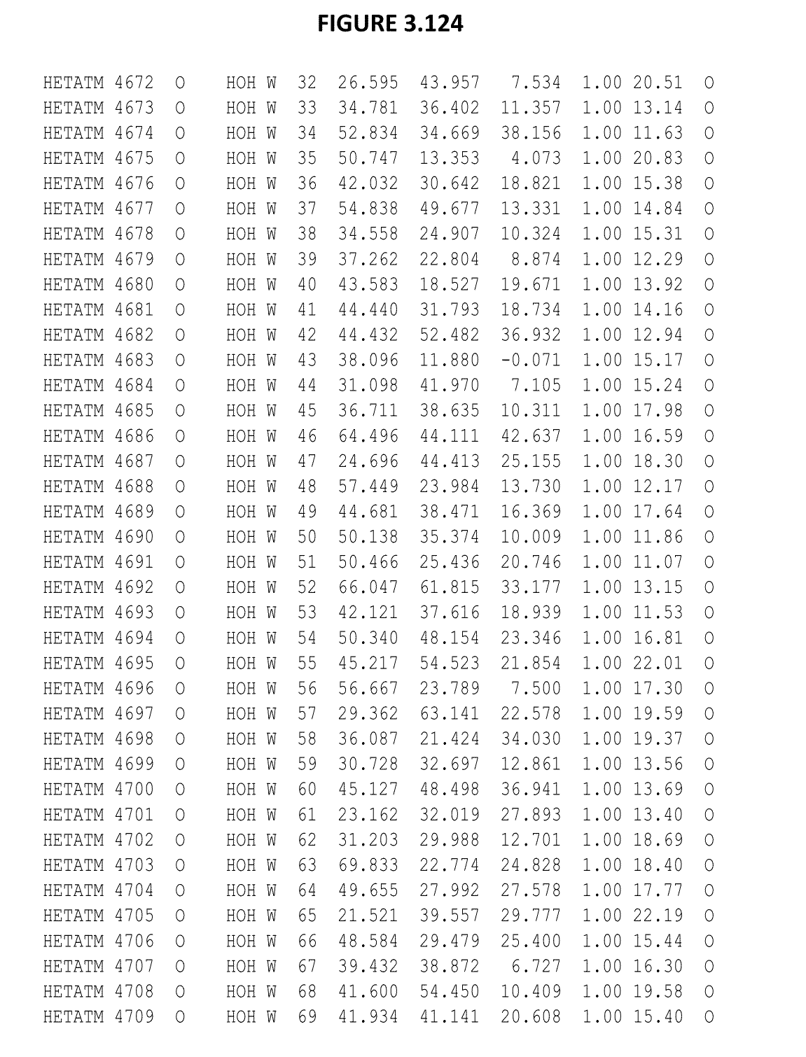

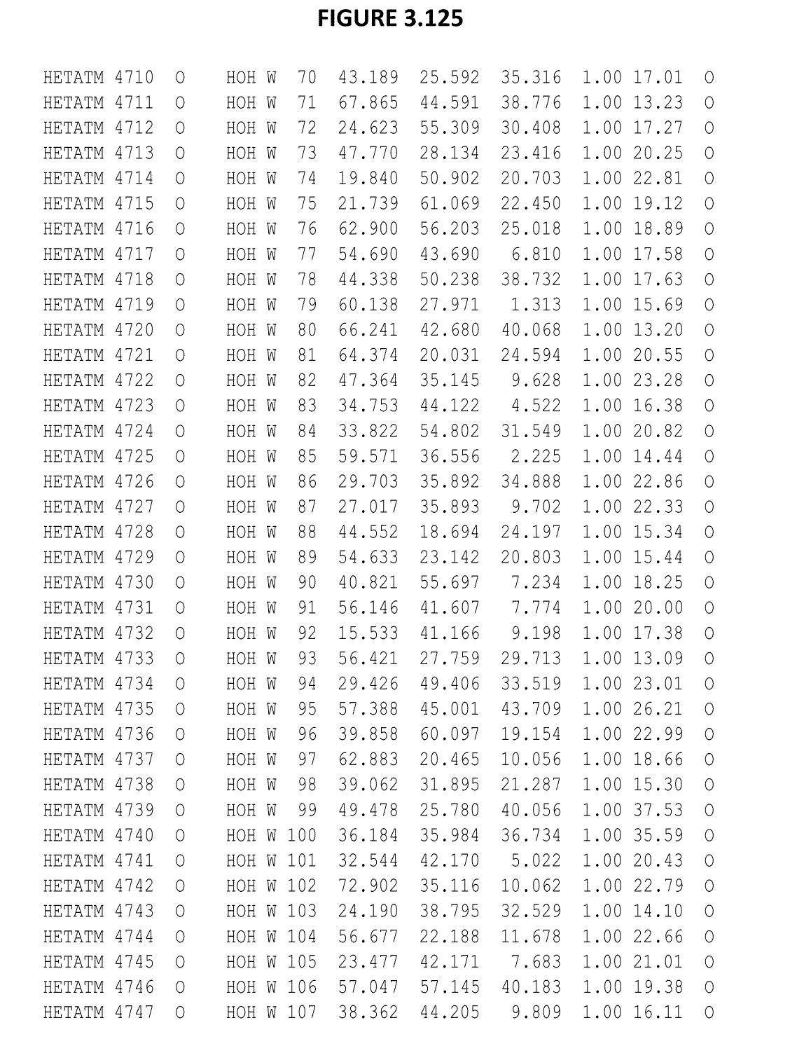

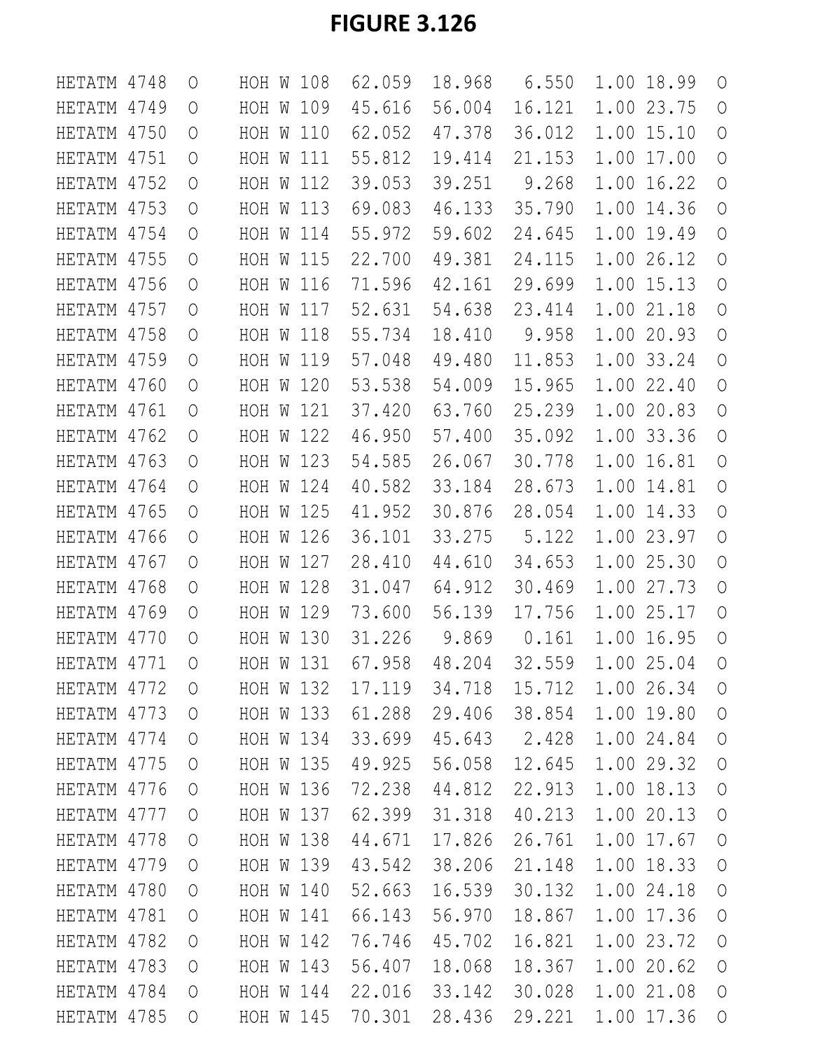

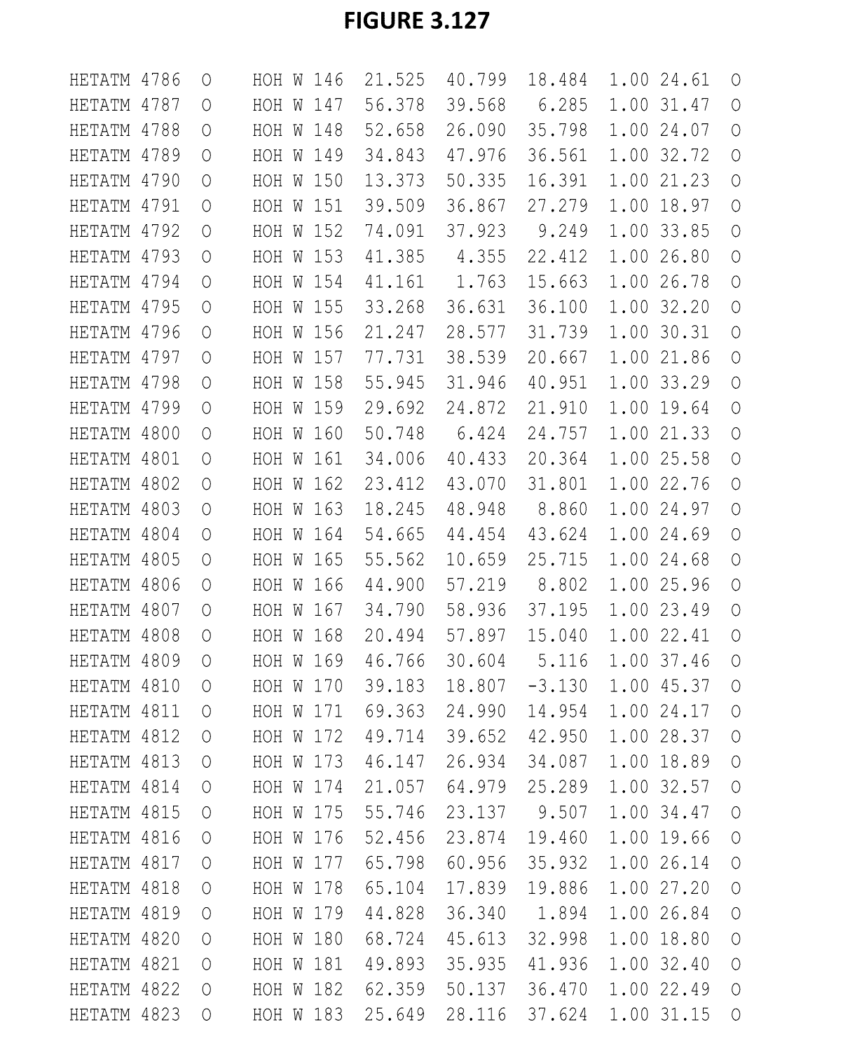

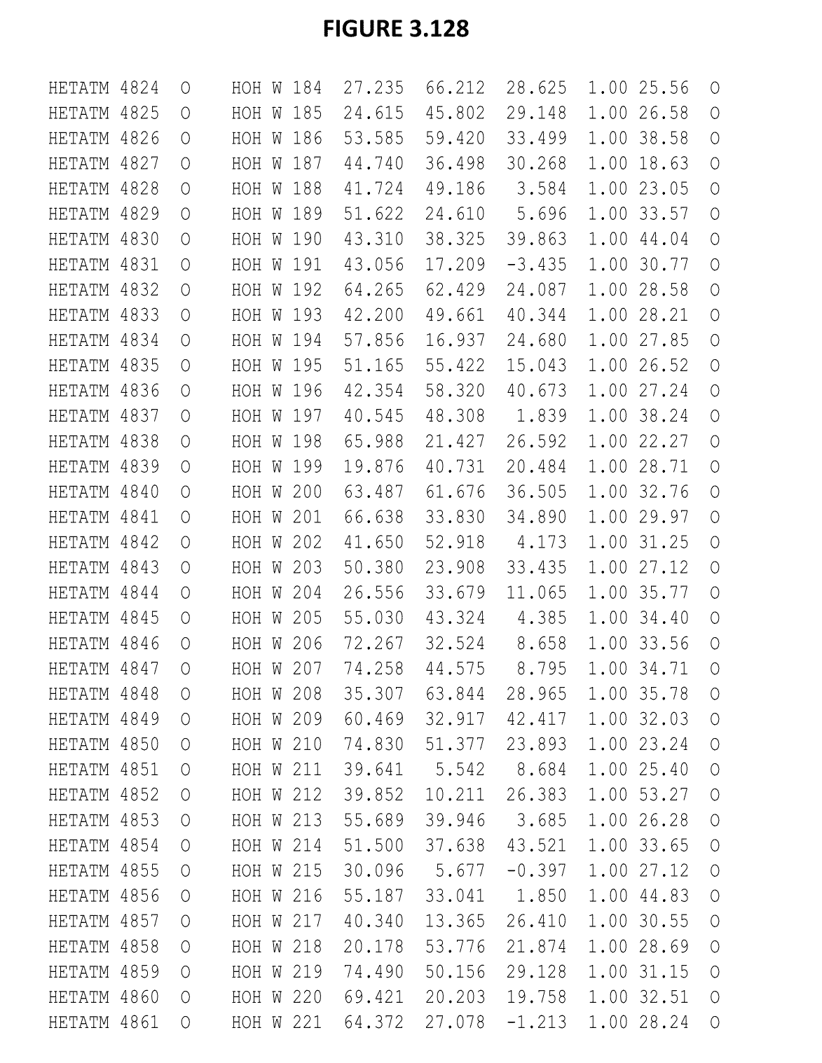

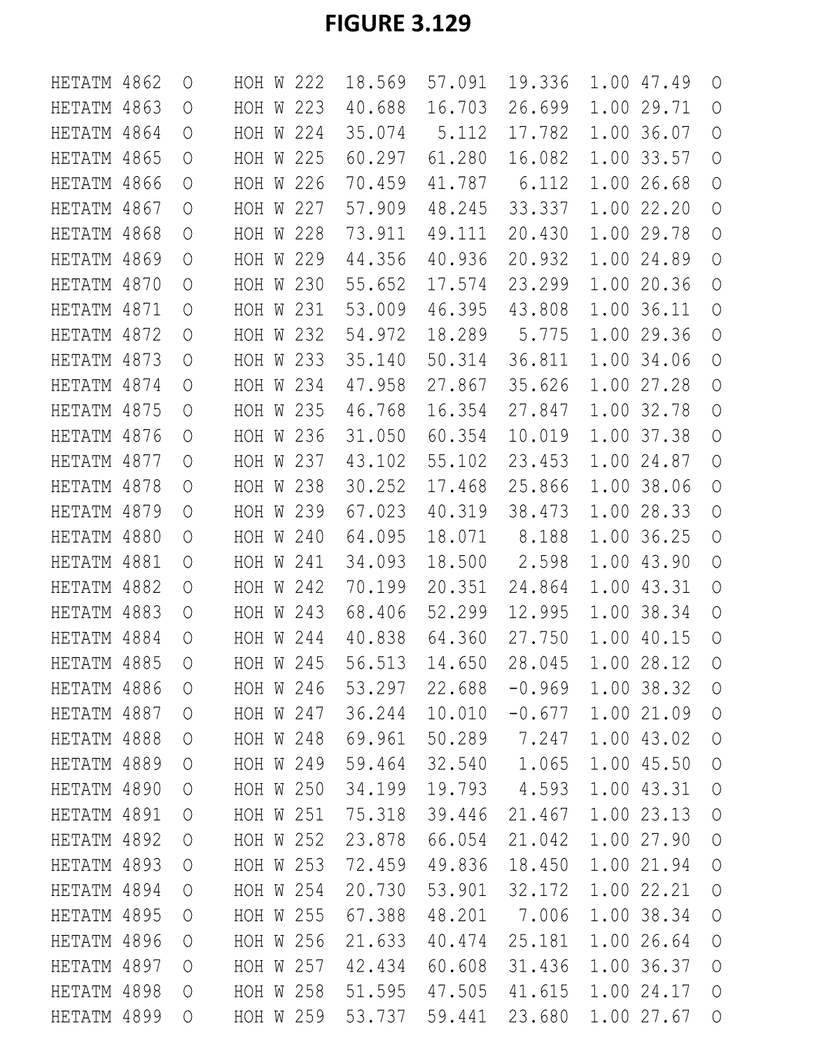

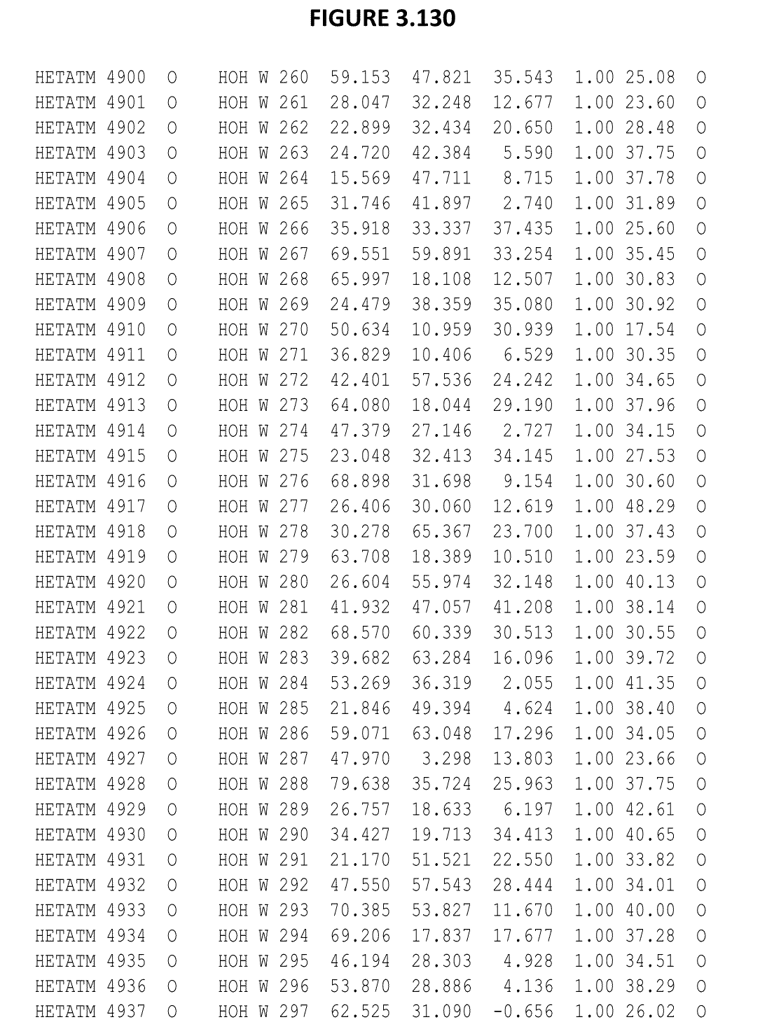

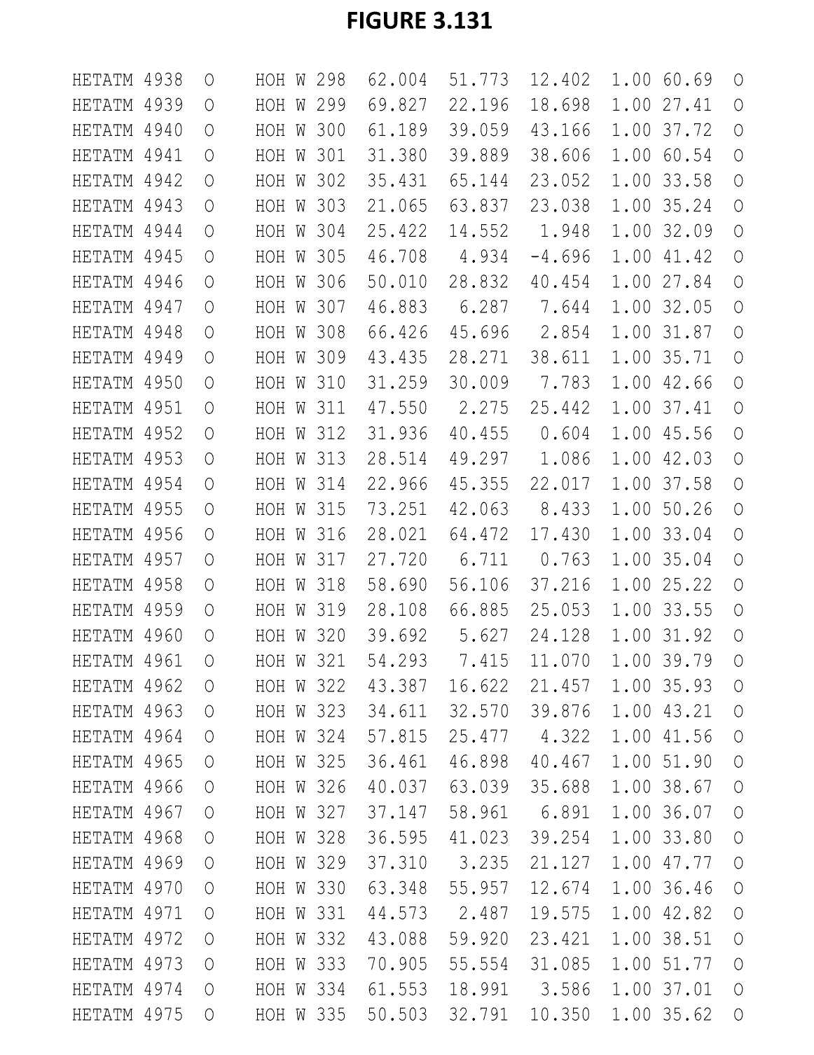

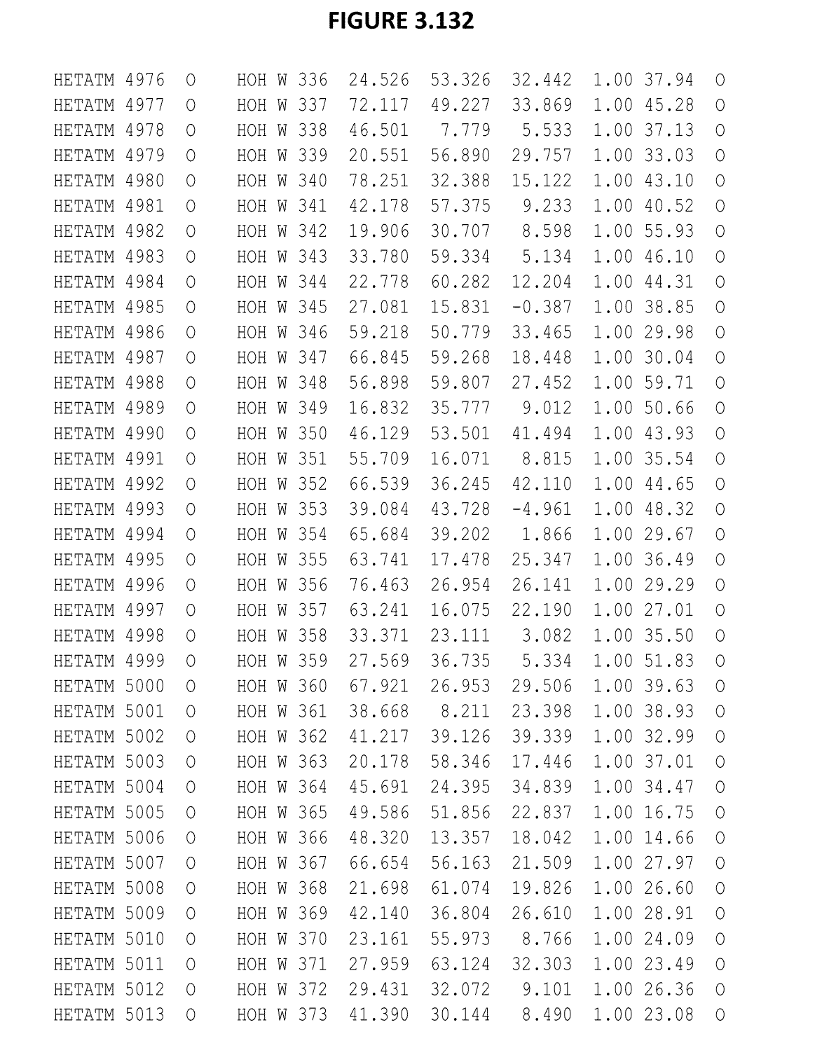

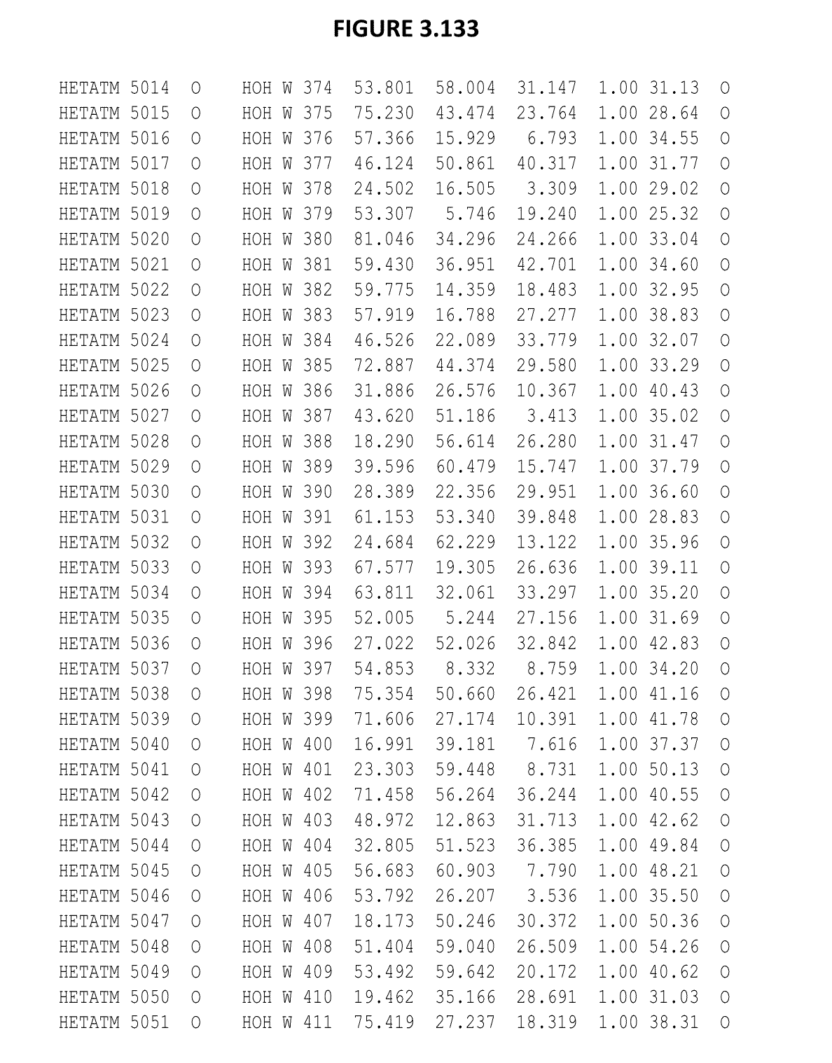

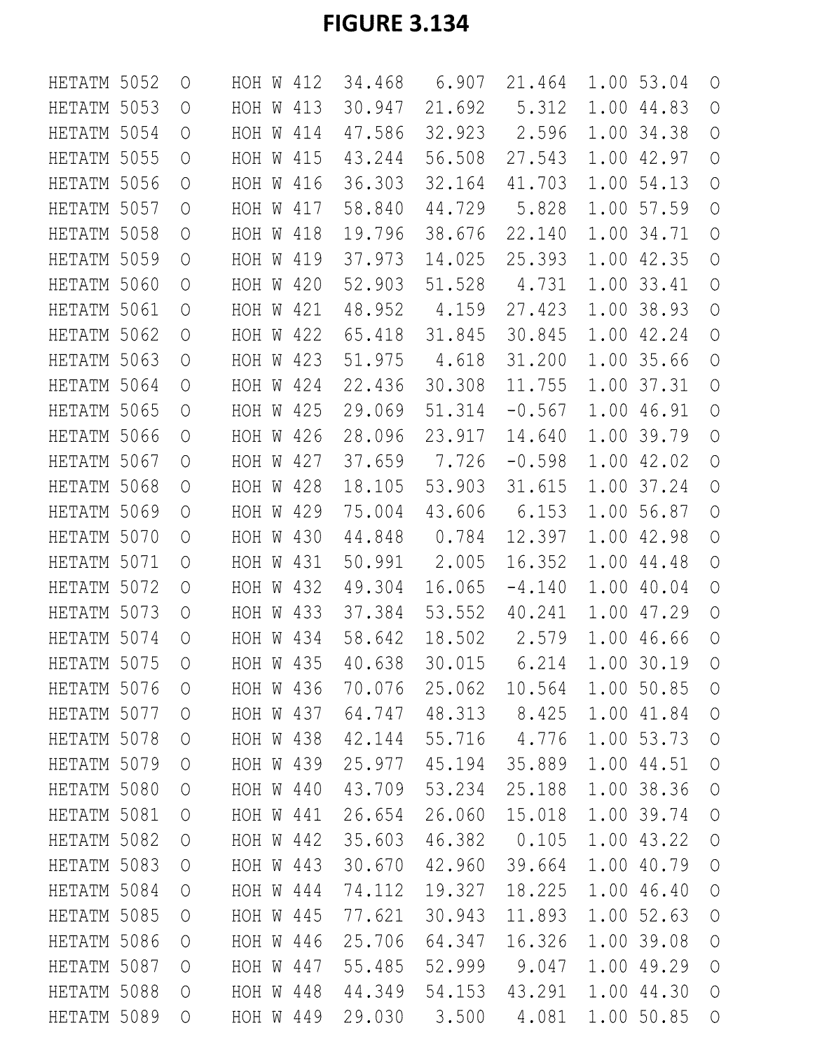

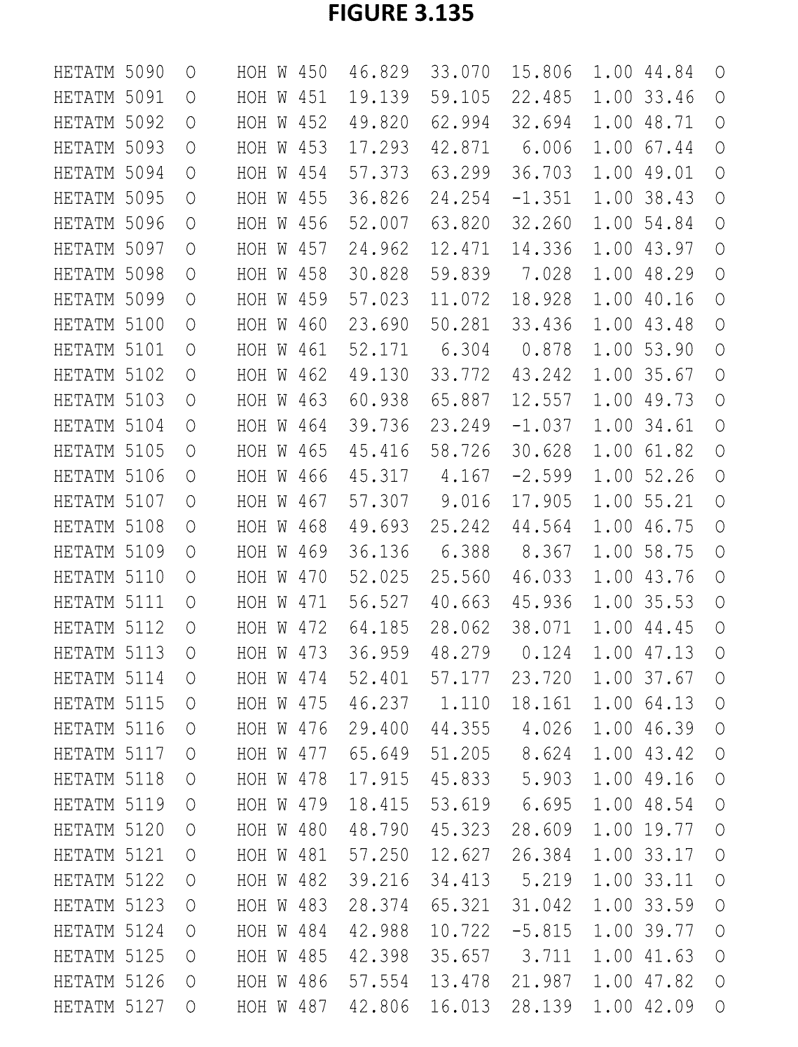

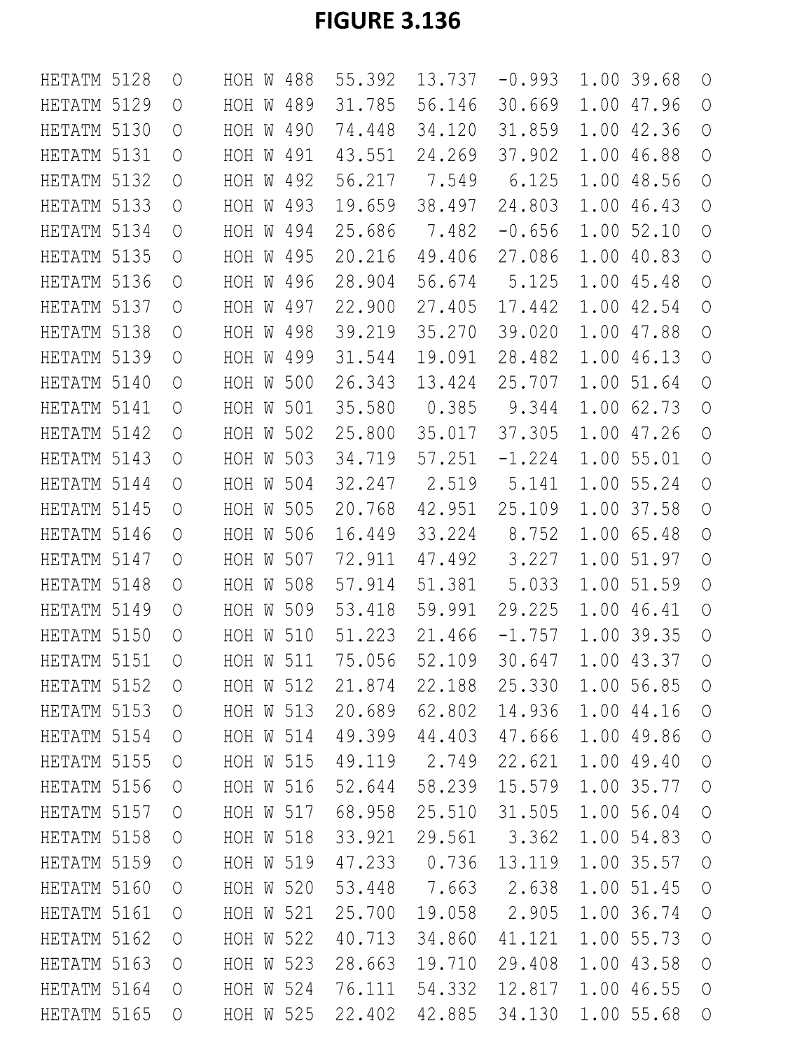

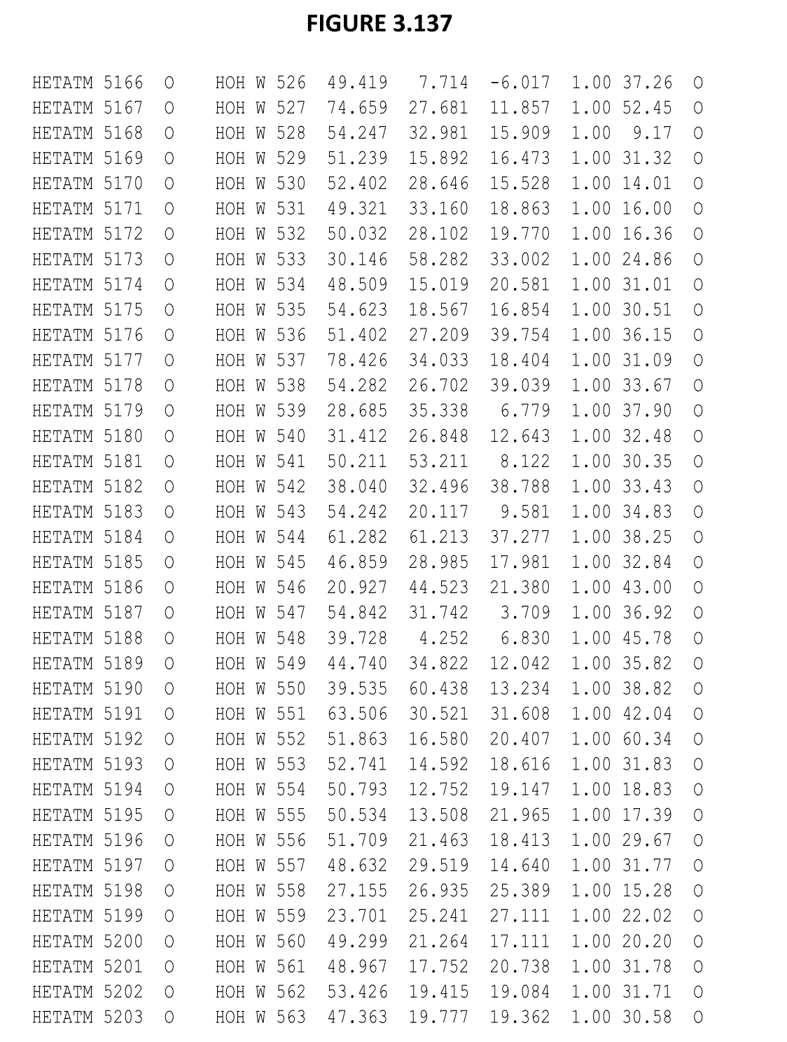

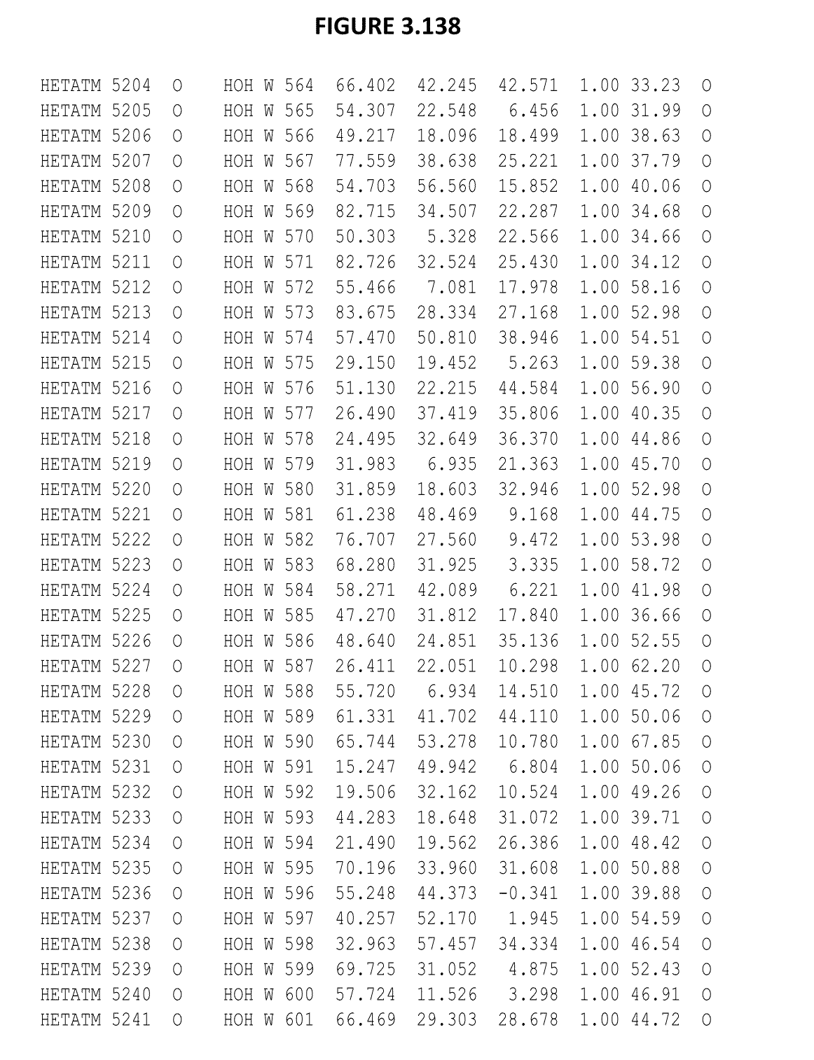

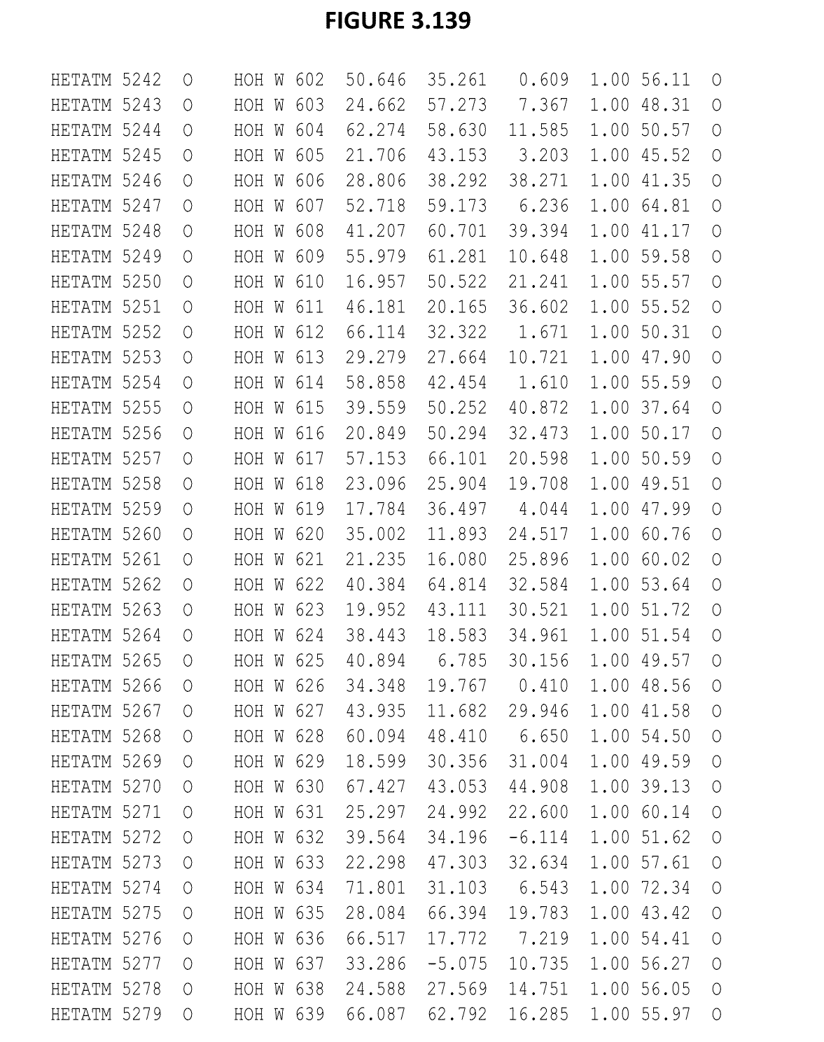

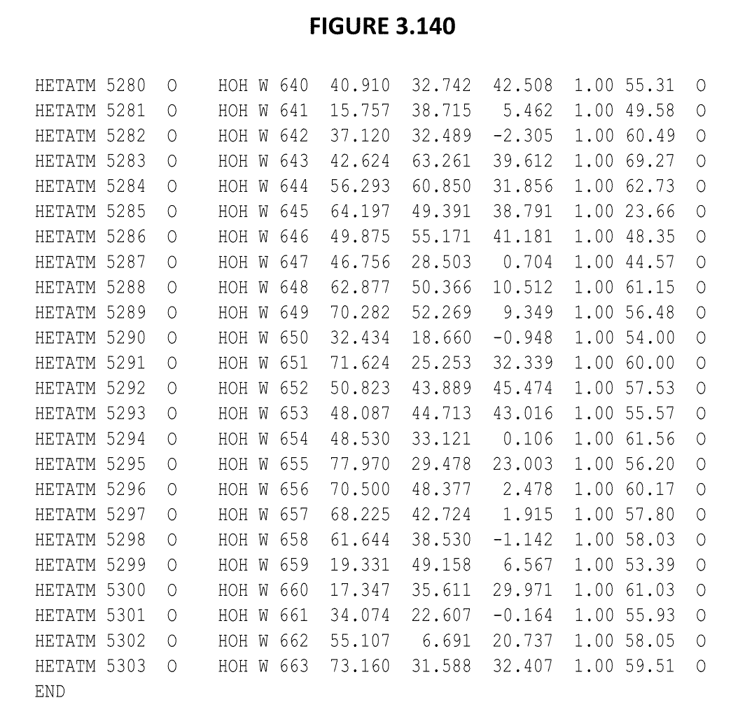

Mostly preferred modes of the invention pertain to the crystals with the aminoacids coordinated as shown in FIG. 2 or 3.

For these are exemplified by this specification and directly allow the analysis of GKRP as based on crystal data.

A second aspect of the invention resides in polynucleotides encoding for GKRP variants with at least one nucleotide different from SEQ ID NO. 1, 3 or 5 (other than wildtype) as defined above.

The present specification discloses nucleotide sequences for GKRP from the different organisms of human, mouse and rat under SEQ ID NO. 1, 3 and 5, respectively. Accordingly the teaching of the invention cannot refer directly to the pre-described wildtype sequences themselves. However, all variants, i.e. not-wildtype sequences developed in context with the invention aim at the creation of GKRP crystals or of crystals of appropriate deletion mutants for gaining useful crystals, the respective rationale explained above.

Accordingly, all nucleotide sequences coding for GKRP or GKRP deletion mutants that support the invention discussed here, also make up parts of the invention themselves. This becomes very clear from the examples which explain that certain mutants had to be created in order to receive sufficient amounts of the protein by expression via an appropriate system and to receive crystals. On the other hand, the GKRP variants described above can not be produced without the respective nucleotides coding for them which motives an equal protection for the polynucleotides encoding for GKRP variants with at least one nucleotide different from SEQ ID NO. 1, 3 or 5 (other than wildtype) as defined above

One mode of this aspect of the invention pertains to such polynucleotide comprising one or more codons optimized for an expression system, preferably one or more codons optimized for the expression in an eukaryotic expression system, more preferred for the expression in mammalian or insect cells.

This is supported by the fact that sufficient amounts of crystallizable protein are best produced by transgenic expression in an appropriate host. To ease this expression it preferred to adapt the sequence to the respective codon usage. This is exemplified by SEQ ID NO. 7 and SEQ ID NO. 15 which are sequences optimized for the codon usage in insect cells that can be used for expression, as exemplified by example 1.

Accordingly preferred are polynucleotides according this aspect encoding for a GKRP variant selected from SEQ ID NO. 8, 10, 12 and 14 or the polynucleotide of SEQ ID NO. 7, preferably a polynucleotide selected from SEQ ID NO. 7, 9, 11, 13 and 15, most preferred the polynucleotide of SEQ ID NO. 15.

For these aminoacid sequences have turned out to give useful crystals that are accessible by appropriate nucleotide sequences.

A further preferred subject of the invention is a GKRP variant with at least one aminoacid different from SEQ ID NO. 2, 4 or 6 (other than wildtype) as defined before.

A further preferred subject of the invention is such a GKRP variant selected from hGKRP_C-His (SEQ ID NO. 8), hGKRP_C-His_K326T/K327T (SEQ ID NO. 10), mGKRP_C-His (SEQ ID NO. 12) and rGKRP_C-His (SEQ ID NO. 14), preferably hGKRP_C-His_K326T/K327T (SEQ ID NO. 10).

A further preferred subject of the invention is a vector comprising a Polynucleotide encoding for a GKRP or GKRP variant according to the definitions above.

A further preferred subject of the invention is such a vector which is an expression vector.

A further preferred subject of the invention is a host cell comprising a polynucleotide encoding for a GKRP or GKRP variant according to the definitions above.

A further preferred subject of the invention is such a host cell, expressing the GKRP or GKRP variant, preferably an eukaryotic host cell, more preferred a mammalian or insect cell, mostly preferred a cell derived from Spodoptera frugiperda.

A further preferred subject of the invention is a process for the crystallization of a GKRP or GKRP variant comprising the steps

(1.) purification of the protein and
(2.) crystallization of the purified protein.

A further preferred subject of the invention is such a process for the crystallization of a GKRP or GKRP variant as defined above.

A further preferred subject of the invention is such a process, wherein for step (2.) the purified protein is complexed with a low molecular weight binding ligand in the active site, preferably with a low molecular weight binding ligand selected from Fructose-1-Phosphate (F1P), Fructose-6-Phosphate (F6P), Orthophosphate (Pᵢ) and Sorbitol-6-Phosphate (S6P), preferably Fructose-1-Phosphate (F1P) or Orthophosphate (Pᵢ).

A further preferred subject of the invention is such a process, characterized by the sitting drop vapour diffusion method for step (2.).

A further preferred subject of the invention is such a process wherein step (2.) is performed between 17.5 and 22.5° C. and preceded by a preincubation of the solution of the purified GKRP or GKRP variant at 12-16 mg/ml in buffer-P2 (25 mM Hepes pH 7.4, 50 mM KCl, 1 mM MgCl₂, 2 mM DTT) supplemented with 5 mM fructose-1-phosphate (F1P) for 0.5 to 1.5 h at 3 to 5° C.

A further preferred subject of the invention is such a process wherein step (2.) is performed between 17.5 and 22.5° C. and preceded by a preincubation of the solution of the purified GKRP or GKRP variant at 12-16 mg/ml in buffer-P2 (25 mM Hepes pH 7.4, 50 mM KCl, 1 mM MgCl₂, 2 mM DTT) for 0.5 to 1.5 h at 3 to 5° C.

A further preferred subject of the invention is such a process according to one or more of claims 30 to 32, wherein the solution of the GKRP or GKRP variant and a reservoir solution consisting of 14.4% PEG 8.000, 20% Glycerin, 0.16 M Calcium acetate and 0.08 M Cacodylate pH 6.5 are mixed in a volume ratio of 1:1 resulting in the mixture of the sitting drop, preferably by a mixture of 0.75 to 1.25 μl each.

A further preferred subject of the invention is such a process according to one or more of claims 27 to 33, wherein the crystals resulting from step (2.) are flash frozen with the mother liquor serving as cryo-protectant, preferably in a nitrogen stream below 150 K.

A further preferred subject of the invention is such a crystal of a GKRP or GKRP variant produced according to one or more of the processes defined above.

A further preferred subject of the invention is the use of a crystal of a GKRP or GKRP variant according to the definitions above for the identification of a low molecular weight chemical molecule or protein that binds to GKRP.

A further preferred subject of the invention is such a use, wherein the binding low molecular chemical molecule or protein binds to the active site of GKRP and/or to the contact site of its respective Glucokinase (GK), and preferably inhibits the enzymatic activity of the GKRP and/or interferes with the interaction of the GKRP with its respective GK.

A further preferred subject of the invention is such a use, wherein the active site of GKRP is defined by one or more of the aminoacid residues or H₂O molecules selected from Arg518, Leu515, His351, Lys514, Asn512, Ser183, Glu153, Glu348, Gly181, Ala184, Ser179, Arg259, Gly107, Val180, Thr109, Ser110, Ser258, Gly108, Ile178, a H₂O molecule complexed by Arg518 and His351, a H₂O molecule complexed by Gly153 and Ser183, a H₂O molecule complexed by Arg259 and Ser258, a H₂O molecule complexed by Thr109 and a H₂O molecule complexed by Gly107 and Ile178, preferably by one or more of the aminoacid residues selected from Lys514, Asn512, Glu153, Gly181, Ser179, Val180, Gly107, Ser110, Thr109, Glu348, wherein all numbers refer to SEQ ID NO. 2.

A further preferred subject of the invention is such a use, wherein the binding low molecular chemical molecule or protein binds partially or completely to another site than the active site of GKRP as defined by claim 37 but nonetheless interferes with the enzymatic activity and/or the interaction with the respective Glucokinase (GK).

A further preferred subject of the invention is such a use, wherein the binding of the low molecular weight chemical molecule or protein induces a conformational change and/or stabilizes a conformation of the GKRP that negatively affects the interaction with the respective Glucokinase (GK) in comparison to the conformation of the GKRP free from the same low molecular chemical molecule or protein.

A further preferred subject of the invention is such a use, wherein the identification takes place by the cocrystallization with the low molecular weight chemical molecule or protein, preferably according to a process as defined above, with the low molecular weight chemical molecule or protein instead of the otherwise complexed low molecular weight binding ligands, preferably instead of the complexed low molecular weight binding ligands mentioned above.

A further preferred subject of the invention is such a use, wherein the identification takes place by soaking of the crystal with a solution comprising the low molecular weight chemical molecule or protein.

A further preferred subject of the invention is such a use, wherein the identification takes place by a computer-aided modelling program for the design of binding molecules, preferably starting from the structure of hGKRP_C-His_K326T/K327T (SEQ ID NO. 12) and the low molecular weight binding ligand in the active site selected from Fructose-1-Phosphate (F1P; FIG. 2) and Orthophosphate (Pᵢ; FIG. 3).

A further preferred subject of the invention is such a use, wherein the low molecular weight chemical molecule is selected from a sugar and/or phosphate containing compound.

A further preferred subject of the invention is such a use, wherein the protein is selected from antibodies.

A further preferred subject of the invention is such a use, wherein the low molecular weight chemical molecule or protein is further characterized by a biochemical assay before, after or in parallel to the use of the crystal.

A further preferred subject of the invention is such a use, wherein the biochemical assay is characterized by the presence of glucokinase (GK; coupled assay), preferably an assay that measures the activity of glucokinase.

### EXAMPLES

### Example 1

### Molecular Biology for the Production of Human GKRP

The gene encoding for human GKRP (SWISS-Prot. entry Q14397; hGKRP) is disclosed in SEQ ID NO. 1, the derived aminoacid sequence in SEQ ID NO. 2. In order to allow an efficient expression and biotechnological production, the cDNA was codon-optimised for expression in insect cells by adapting the codon usage to the one of Spodoptera frugiperda genes, as taught by Sharp and Li (1987); Nucleic Acids Res., 15 (3), 1281-1295. Accordingly, the following sequence motifs were avoided: internal TATA-boxes, chi-sites and ribosomal entry sites, AT-rich or GC-rich sequence stretches, ARE, INS, CRS sequence elements, repeat sequences and RNA secondary structures, (cryptic) splice donor and acceptor sites, branch points; additionally a Kozak sequence was introduced to increase translational initiation and two STOP codons were added to ensure efficient termination. The resulting gene possesses an average GC content of about 60%, basically no negative cis-acting sites (such as splice sites, poly(A) signals, etc) which may negatively influence expression, and a codon usage adapted to the bias of Spodoptera frugiperda resulting in a high codon adaptation index according to Sharp and Li of about 0.97.

Further it was flanked by attB1 (upstream) and attB2 (downstream) sites (SEQ ID NO. 17 and 18) and cloning was performed using the commercially available Gateway® cloning system into vector pDONR221® and subsequently into pDEST8® vector (all commercially available by e.g. Invitrogen, Groningen, Netherlands; comparable cloning systems could be used as alternatives.)

The resulting open reading frame encodes for hGKRP with a C-terminal LEHHHHHH octapeptide added, referred to as hGKRP_C-His. It is disclosed in SEQ ID NO. 7. The deduced aminoacid sequence is disclosed in SEQ ID NO. 8, which shows that the protein according to this example is identical with the wildtype enzyme, plus the additional C-terminal octapeptide. This optimized gene is expected to allow high and stable expression rates of hGKRP_C-His and related proteins in Spodoptera frugiperda and other eukaryotic expression systems, especially insect cells.

The hGKRP_C-His_K326T/K327T double mutant is identical to hGKRP_C-His with the amino acids lysine in position 326 and lysine in position 327 both mutated to threonine (SEQ ID NO. 9, 10). After constructing the corresponding bacemids by the BAC-to-BAC® system (Invitrogen; comparable cloning systems could be used as alternatives), the proteins were expressed in High FIVE® cells for 72 h at 27° C. The cells were harvested by centrifugation and frozen at −70° C.

Mouse GKRP (mGKRP, deduced from genome data and disclosed in SEQ ID NO. 1) and rat (rGKRP; SWISS-Prot. entry Q07071) have been prepared as described for hGKRP. The nucleotide sequence used for the molecular biology production as well as the deduced aminoacid sequence (identical with the wildtype aminoacid sequence supplemented with the C-terminal histidine rich oligopeptide) are given in SEQ ID NO. 11 and 12 (mouse) and SEQ ID NO. 13 and 14 (rat), respectively.

### Example 2

### Protein Purification

Purification of hGKRP_C-His

Frozen cells expressing hGKRP_C-His; SEQ ID NO. 7, 8) prepared according to Example 1 were thawed, resuspended in lysis buffer (25 mM Hepes pH 8, 0.1 mM MgCl₂, 500 mM NaCl, Complete EDTA-free protease inhibitor (RocheDiagnostics, Penzberg, Germany; one tablet per 50 ml), 0.2 mM DTT, 3 μg/ml DNAse) and broken by one freeze-thaw cyclus. The lysate was centrifuged for 60 min at 20.000 g. The supernatant (400 ml) was incubated with 9 ml NiNTA agarose beads in buffer-A (50 mM Na₂HPO₄ pH 8.0 500 mM NaCl) for 60 min at 4° C. Beads were then washed with 40 ml buffer-A and subsequently with 2% buffer-B (50 mM Na₂HPO₄ pH 7.0, 500 mM NaCl, 0.5 M Imidazol, 5 mM DTT) in buffer-A until absorbance at 280 nm (A280) of the eluate returned to baseline (approx. 40 mL). GKRP was then eluted from the beads in 20 mL buffer-B. The eluted protein was concentrated and further purified by size exclusion chromatography (Superdex 200, Amersham) in buffer-S (100 mM Hepes pH 7.4, 200 mM KCl, 1 mM MgCl₂, 2 mM DTT).

Purification of hGKRP_C-His_K326T/K327T

The double mutant hGKRP_C-His_K326T/K327T (SEQ ID NO. 9, 10) was expressed and purified following the same protocol.

Purification of mGKRP and rGKRP

The C-terminally modified GKRP from mouse (mGKRP; SEQ ID NO. 3, 4) and rat (rGKRP; SEQ ID NO. 5, 6) were expressed and purified following the same protocol.

All resulting proteins could be purified in mg amounts, were homogenous according to ESI-MS and size exclusion chromatography and could be concentrated to more than 20 mg/ml.

### Example 3

### Enzymatic Characterization

Enzymatic Activity of hGKRP

GKRP preparations according to examples 1 and 2 have been examined with respect to their enzymatic activity. The applied enzymatic assay measures the effect of GKRP on glucokinase activity in the form of a glucose-6-phosphate dehydrogenase coupled assay at room temperature which is a modification of the method described by Van Schaftingen and Brocklehurst et al. (Van Schaftingen, E. (1989): A protein from rat liver confers to glucokinase the property of being antagonistically regulated by fructose 6-phosphate and fructose 1-phosphate; Eur. J. Biochem., 179, 179-184; Brocklehurst, K. J., Davies, R. A. and Agius, L. (2004): Differences in regulatory properties between human and rat glucokinase regulatory protein; Biochem. J., 378, 693-697).

The reaction mixture contained 150 mM KCl, 100 mM Hepes, 1 mM ATP, 1 mM MgCl2, 2 mM NADP⁺, 2 mM dithiothreitol, pH 7.4, 5 units/ml glucose-6-phosphate dehydrogenase, 0.5 mg/ml BSA, 10 mM glucose, 6 μM fructose 6-P, 15 nM human liver glucokinase and 100 nM of the respective GKRP. The enzymatic reaction was started by the addition of ATP and glucose. The increase in optical density was measured at a wavelength 340 nm over 10 minutes. From these kinetic data, the slope was calculated and graphically depicted.

As a result it was found that in line with Brocklehurst et al. the recombinant hGKRP_C-His alone is capable of inhibiting the apparent GK activity in a dose-dependent manner by inducing an inactive GK-GKRP complex. When dosed in excess over GK (final concentration 15 nM), an almost complete inhibition of the apparent GK enzymatic activity by >90% was observed, indicating a very pronounced shift of the equilibrium towards the inactive GK-GKRP complex (IC₅₀=124±9 nM).

A control experiment under identical conditions was performed in which the reaction buffer has been added 6 μM fructose 6-phosphate. As a result it was found that the addition of 6 μM fructose 6-phosphate apparently induced a higher affinity of the F6P-bound GKRP protein for GK binding, as the inhibition of GK activity already occurred at lower GKRP concentrations (IC₅₀=74±6 nM). In further experiments it was found that the effect of F6P on the formation of the inactive GK-GKRP-F6P complex is dose-dependent.

Enzymatic Activity of Expressed hGKRP_C-His_K326T/K327T

In comparison to hGKRP_C-His, hGKRP_C-His_K326T/K327T is equally capable of decreasing the apparent activity of GK in the reaction mixture by inducing the formation of the inactive complexes both alone but also in the presence of 6 μM F6P (IC₅₀=116±10 nM and 71±7 nM, respectively).

Competitive Binding of fructose-1-phosphate and fructose-6-phosphate

The ability of F1P to compete with the binding of F6P as has been suggested by Veiga-da-Cunha and Van Schaftingen (Veiga-da-Cunha, M. and Van Schaftingen, E. (2002): Identification of fructose 6-phosphate- and fructose 1-phosphate-binding residues in the regulatory protein of glucokinase; J. Biol. Chem., 277, 8466-8473).

This effect was investigated using both hGKRP_C-His as well as hGKRP_C-His_K326T/K327T. In the presence of 6 μM F6P, increasing concentrations of F1P are able to dose-dependently increase the apparent GK activity in the reaction mixture. The concentrations of F1P needed to drive the equilibrium from the inactive GK-GKRP-F6P complex towards free GK are comparable using either wild hGKRP_C-His (EC₅₀=6.28±1.07 μM) or hGKRP_C-His_K326T/K327T (EC₅₀=5.08±1.38 μM). This again indicates that the major functional properties of hGKRP_C-His_K326T/K327T according to the invention, especially to bind to GK, to function as a regulator of GK activity and to be regulated by its endogenous regulatory molecules F6P and F1P in a competitive way, are fully retained and are comparable to hGKRP_C-His.

In summary, it was shown by these experiments that hGKRP (wildtype) as well as the variants hGKRP_C-His and hGKRP_C-His_K326T/K327T, all produced according to the foregoing example are fully active GKRPs.

### Example 4

### Crystallisation of a hGKRP_C-His_K326T/K327T-fructose-1-phosphate Complex

Crystals of hGKRP_C-His_K326T/K327T in complex with fructose-1-phosphate (hGKRP_C-His_K326T/K327T-F1P) were grown at 20° C. by the publicly known sitting drop vapour diffusion method (McPherson, A. (1982) Preparation and Analysis of Protein Crystals, Wiley Interscience, New York). Prior to crystallization, hGKRP_C-His_K326T/K327T-F1P was prepared by incubating hGKRP_C-His_K326T/K327T at 12-16 mg/ml in buffer-P2 (25 mM Hepes pH 7.4, 50 mM KCl, 1 mM MgCl₂, 2 mM DTT) supplemented with 5 mM fructose-1-phosphate (F1P) for 1 h at 4° C. Typical crystallization drops were formed by mixing 1 μl hGKRP_C-His_K326T/K327T-F1P and 1 μl reservoir solution consisting of 14.4% PEG 8.000, 20% Glycerin, 0.16 M Calcium acetate and 0.08 M Cacodylate pH 6.5. Crystals were flash frozen in a 100 K nitrogen stream, with the mother liquor serving as cryo-protectant.

### Example 5

### Crystallisation of a hGKRP_C-His_K326T/K327T-phosphate Complex

hGKRP_C-His_K326T/K327T in complex with phosphate (hGKRP_C-His_K326T/K327T-P) was crystallized as described for hGKRP_C-His_K326T/K327T-F1P, but without the addition of 5 mM F1P. The reservoir solution consisted of 20% PEG 3350, 0.1 M Tris pH 8.0. Phosphate was not explicitly added, but residual phosphate from the previous NiNTA purification step remained bound to the protein (see below).

### Example 6

### Data Collection, Structure Solution and Refinement

All diffraction data were collected at 100 K on the PX-1 beamline at the SLS (Villigen, Switzerland) and processed with XDS according to Kabsch, W. (2010): XDS; Acta Cryst. D66, 125-132. Statistics of the data processing are shown below in table 1. An initial high resolution dataset was used for molecular replacement trials and SIR-AS phasing. For initial molecular replacement trials models were used that have been identified with the help of the HHpred server described by Söding, J. et al. (2005): The HHpred interactive server for protein homology detection and structure prediction; Nucleic Acids Res. 33, W244-W248.

The structure of hGKRP_C-His_K326T/K327T-F1P was solved with the SIR-AS method. For derivatization a crystal of hGKRP_C-His_K326T/K327T-F1P was soaked for 3 days in an artificial mother liquor where the calcium acetate was exchanged for 160 mM EuAc3. Identification of the heavy atom substructure, phasing and density modification were performed with program AutoSharp® (Global Phasing Ltd.). The model of hGKRP_C-His_K326T/K327T was semiautomatically built with arp-warp (Morris, R. et al. (2003): ARP/wARP and automatic interpretation of protein electron density maps; Methods Enzymol., 374, 229-244). Subsequently missing residues as well as fructose-1-phosphate were manually built using the computer program Coot (Emsley, P. and Cowtan, K. (2004): Coot: model-building tools for molecular graphics; Acta Cryst. D60, 2126-2132) and the resulting model was improved by iterative rounds of manual rebuilding and refinement with the computer program Buster® (Global Phasing Ltd.).

Final refinement was performed against the dataset of a second hGKRP_C-His_K326T/K327T crystal. The final model has been completed to residues 6 to 606 of hGKRP_C-His_K326T/K327T, one fructose-1-phosphate molecule, one Ca²⁺ ion and 700 water molecules. N- and C-termini as well as a short surface loop (residues 64-68) are disordered (and therefore not included in the coordinates given in FIG. 2). As defined by computer program MolProbity® (Davis, I. W. et al. (2007): MolProbity: all-atom contacts and structure validation for proteins and nucleic acids; Nucleic Acids Res., 35, W375-W383) there are 98.6% of residues in the most favored regions of the Ramachandran plot and 1.0% in additionally allowed regions. The hGKRP_C-His_K326T/K327T phosphate complex (hGKRP_C-His_K326T/K327T-P) was solved by difference fourier methods and refined as above. The final statistics for the models are listed in table 4.

TABLE 4
Data collection and refinement of hGKRP_C-His_K326T/K327T-
Fructose-1-phosphate complex (F1P) and hGKRP_C-
His_K326T/K327T-phosphate complex (Phosphate)

| | | |
|---|---|---|
| Data set | F1P | Phosphate |

Data collection ¹

| | | |
|---|---|---|
| Wavelength (Å) | 0.960 | 0.910 |
| Space Group | P2₁2₁2₁ | P2₁2₁2₁ |
| Unit cell dimensions | 61.0 | 60.8 |
| a, b, c (Å) | 72.3 | 72.2 |
| | 136.9 | 138.0 |
| Resolution (Å) | 72-1.47 | 69-1.92 |
| Highest Resolution Shell (Å) | 1.53-1.47 | 1.98-1.92 |
| Observed Reflections | 347484 | 306781 |
| Unique Reflections | 102068 | 47132 |
| Completeness (%) | 98.6 (98.2) | 99.9 (100.0) |
| R_{sym} (%) | 5.1 (39.8) | 9.8 (44.1) |
| <I/σ(I)> | 14.7 (3.9) | 17.0 (6.5) |

Refinement

| | | |
|---|---|---|
| R-factor ⁴ (%) | 16.0 | 16.0 |
| R-free ⁴ (%) | 17.7 | 18.6 |
| Number of refined atoms | 5357 | 5086 |
| protein | 4640 | 4635 |
| solvent | 700 | 446 |
| ligands | 17 | 5 |
| Average B-factor (Å²) | 18.6 | 18.1 |

Rms deviation

| | | |
|---|---|---|
| Bond length (Å) | 0.008 | 0.008 |
| Bond angles (°) | 0.96 | 0.98 |

Ramachandran statistics

| | | |
|---|---|---|
| favoured (%) | 98.6 | 98.1 |
| allowed (%) | 1.1 | 1.5 |
| outliers (%) | 0.3 ⁵ | 0.3 ⁵ |

¹ Values in parentheses are for the highest resolution shell.
² R_{sym} = ΣₕₖₗΣᵢ | Iᵢ − <I> |/ΣₕₖₗΣᵢIᵢ
³ R-factor = Σhkl | | Fobs | −k | Fcalc | |/Σhkl | Fobs |, R-free was calculated using 5% of data excluded from refinement.
⁴ The 3 Ramachandran outliers are well defined in the electron density.

The computer program PyMOL® (DeLano Scientific LLC) was used for figure preparation and structural analysis (RMSD calculations and distance measurements). Coordinates are shown in FIG. 2 (hGKRP_C-His_K326T/K327T-F1P) and FIG. 3 (hGKRP_C-His_K326T/K327T-P).

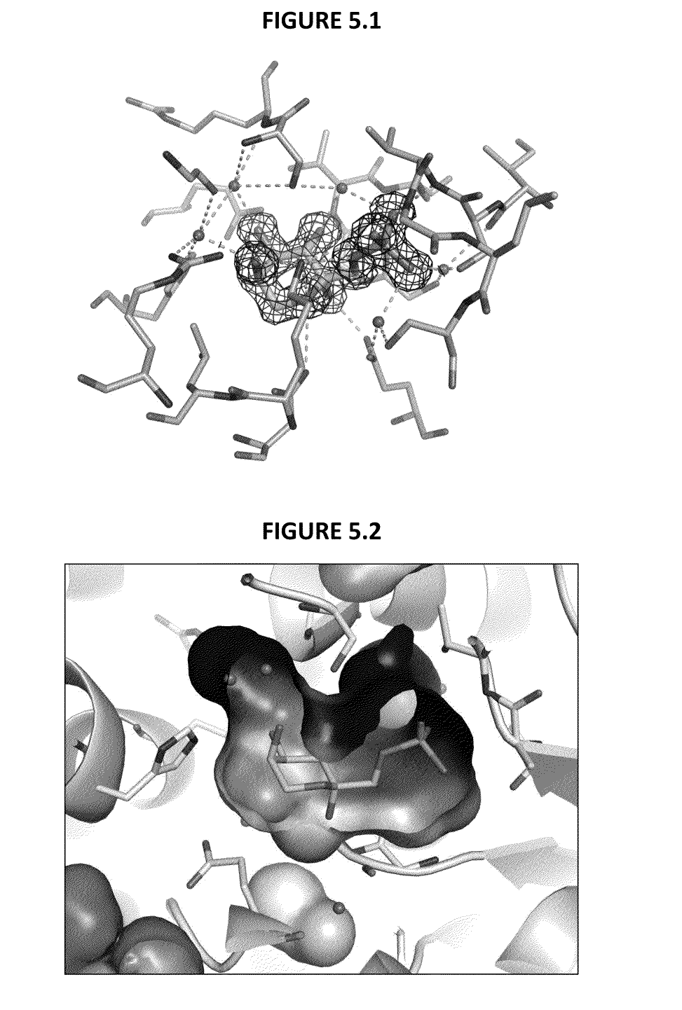

As can be seen, the double mutant hGKRP_C-His_K326T/K327T in complex with Fructose-1-phosphate (hGKRP_C-His_K326T/K327T-F1P) yielded well ordered crystals that diffracted to high resolution. hGKRP_C-His_K326T/K327T-F1P crystallized in space group P2₁2₁2₁ with one molecule in the asymmetric unit. The model of hGKRP_C-His_K326T/K327T-F1P was refined to a resolution of 1.47 Å with an R_{free} value of 17.7% and consists of residues 6-606 of hGKRP_C-His_K326T/K327T (table 1, FIG. 2). A representative portion of the final electron density is shown in FIG. 5.

The data for crystals of hGKRP_C-His_K326T/K327T in complex with phosphate (hGKRP_C-His_K326T/K327T-P) show a clear electron density for a phosphate ion. Further details of that crystal are given in table 1 and FIG. 3.

### Example 7

### Amide Hydrogen (H/D) Exchange Experiment

This experiment was performed to map the potential ligand binding sites via the amide hydrogen exchange behaviour of apo-GKRP in comparison to ligand-bound GKRP.

Amide hydrogen (H/D) exchange was initiated by a 20-fold dilution of 30 pmol hGKRP_C-His with or without ligand into D₂O containing 100 mM HEPES, pD 7.4, 200 mM KCl, 100 mM MgCl₂, and 2 mM DTT and incubated at room temperature.

After various time points (10 sec, 1 min and 30 min), the exchange reaction was quenched by decreasing the temperature to 0° C. and the pH to 2.5 with quench buffer (500 mM KH₂PO₄/H₃PO₄, pH 2.5, 2 M Urea, and 2 mM TCEP). Quenched samples were directly injected into an HPLC setup and analyzed on an electrospray ionization-quadrupole time of flight-mass spectrometer (QSTAR XL, Applied Biosystems) as described by Rist et al. (2003): Mapping temperature-induced conformational changes in the Escherichia coli heat shock transcription factor sigma 32 by amide hydrogen exchange, J. Biol. Chem., 278, 51415-51421.

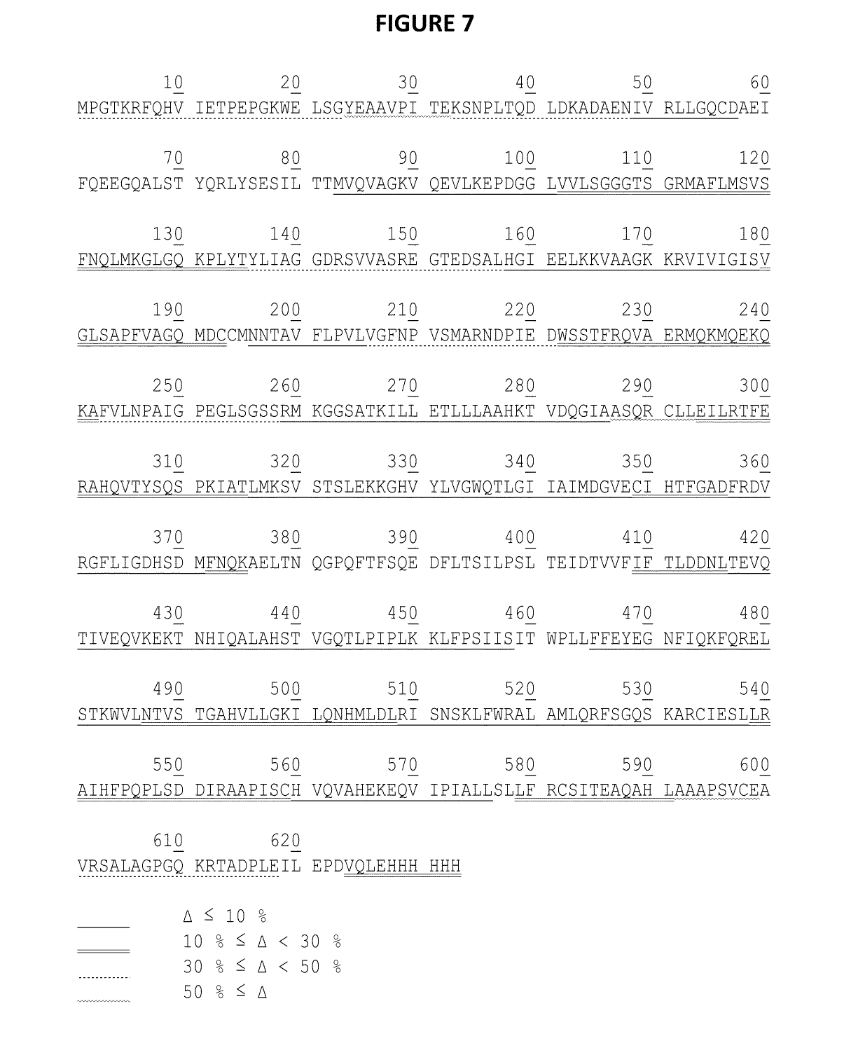

The HPLC setup contained a column (2×20 mm) packed with Poroszyme immobilized pepsin (Applied Biosystems, Darmstadt, Germany). The resulting peptides were trapped on a 0.5×5 mm reversed-phase column (Reprosil-Pur C8) and eluted from the trap column over a 0.5×100 mm Reprosil Gold C8 analytical reversed-phase column (Dr. Maisch, Ammerbuch-Entringen, Germany) with a 8-min gradient directly into the electrospray source. The digestion, desalting, and elution required less than 10 min. The whole setup was immersed in an ice-bath to minimize back-exchange. Peptic peptides of GKRP were identified on the basis of their MS/MS spectra. The deuterium content of the peptides was calculated by using the average mass difference between the isotopic envelopes of the deuterated and the undeuterated peptides. The results are shown in table 5 and visualized in FIG. 7.

TABLE 5
H/D exchange data (exchange time of 1 min) (position
numbering according to SEQ ID NO. 8)

| | | | Hydrogens | No. of amide | |
|---|---|---|---|---|---|
| Peptide | Start | End | exchanged | hydrogens | % exchanged |
| 1 | 2 | 24 | 8.4 | 20 | 42% |
| 2 | 24 | 32 | 3.9 | 7 | 56% |
| 3 | 33 | 48 | 5.6 | 14 | 40% |
| 4 | 49 | 57 | 0.5 | 8 | 6% |
| 5 | 83 | 101 | 0.9 | 17 | 5% |
| 6 | 102 | 116 | 1.7 | 14 | 12% |
| 7 | 117 | 135 | 2.5 | 17 | 15% |
| 8 | 136 | 157 | 7.2 | 21 | 34% |
| 9 | 158 | 179 | 1.0 | 21 | 5% |
| 10 | 180 | 193 | 2.0 | 12 | 17% |
| 11 | 196 | 205 | 0.0 | 8 | 0% |
| 12 | 206 | 213 | 2.2 | 6 | 37% |
| 13 | 214 | 221 | 1.8 | 6 | 30% |
| 14 | 222 | 242 | 3.9 | 20 | 19% |
| 15 | 243 | 258 | 4.0 | 13 | 31% |
| 16 | 259 | 274 | 0.1 | 15 | 0% |
| 17 | 271 | 286 | 1.1 | 15 | 7% |
| 18 | 287 | 293 | 5.8 | 6 | 96% |
| 19 | 294 | 315 | 2.8 | 20 | 14% |
| 20 | 316 | 324 | 0.0 | 8 | 0% |
| 21 | 325 | 342 | 1.0 | 17 | 6% |
| 22 | 343 | 348 | 0.0 | 5 | 0% |
| 23 | 349 | 356 | 1.4 | 7 | 20% |
| 24 | 357 | 371 | 1.3 | 14 | 9% |
| 25 | 360 | 375 | 3.2 | 15 | 21% |
| 26 | 409 | 416 | 1.3 | 7 | 19% |
| 27 | 417 | 435 | 0.0 | 18 | 0% |
| 28 | 436 | 458 | 0.0 | 19 | 0% |
| 29 | 465 | 472 | 0.0 | 7 | 0% |
| 30 | 473 | 486 | 0.0 | 13 | 0% |
| 31 | 487 | 508 | 3.3 | 21 | 16% |
| 32 | 509 | 522 | 0.4 | 13 | 3% |
| 33 | 523 | 538 | 1.3 | 15 | 9% |
| 34 | 539 | 550 | 1.2 | 9 | 13% |
| 35 | 551 | 559 | 1.9 | 7 | 27% |
| 36 | 560 | 576 | 0.9 | 15 | 6% |
| 37 | 579 | 591 | 2.6 | 12 | 22% |
| 38 | 592 | 599 | 3.9 | 6 | 65% |
| 39 | 600 | 618 | 7.4 | 16 | 46% |
| 40 | 624 | 632 | 2.1 | 8 | 27% |

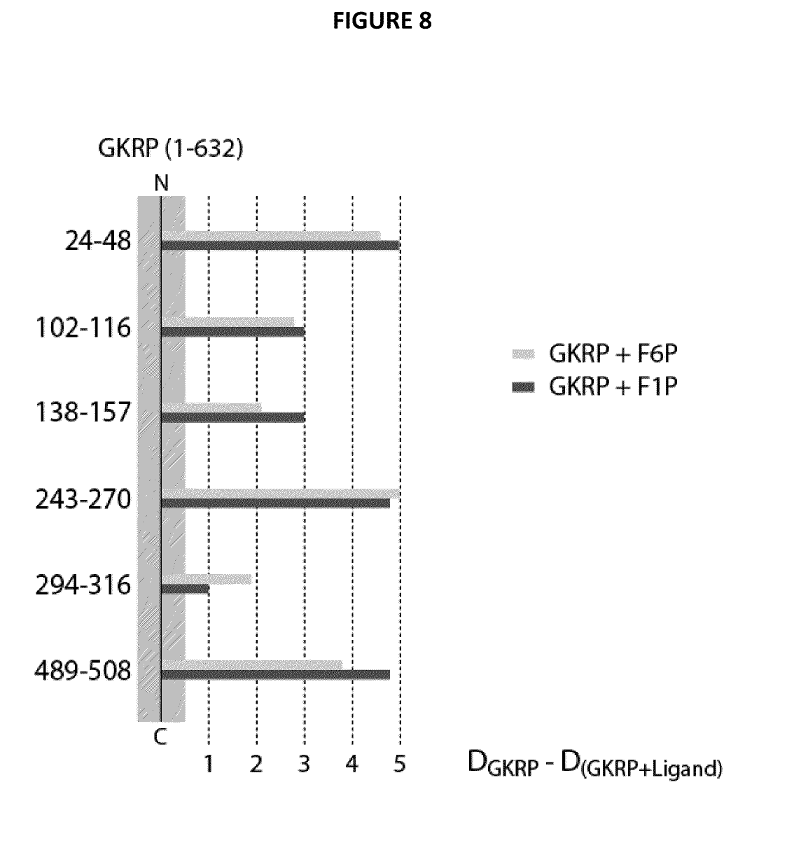

This experiment shows that after 30 min H/D exchange, six regions in GKRP show less deuterium incorporation in the presence of either ligand (F6P or F1P) as compared to apo-GKRP (FIG. 8). Protection against H/D exchange indicates a more compact and less flexible protein fold in the presence of ligand. F6P and F1P show protection against H/D exchange in the same regions in GKRP. This implies that there is one binding site in GKRP for both ligands.

A comparison of the H/D exchange results to the crystallographically observed F1P binding indicates 3 regions (102-116, 136-157 and 243-270) which include residues that are engaged in direct interactions to F1P (FIG. 6). Two loop regions that are not in direct contact to F1P (residues 24-48 of the N-terminus and residues 498-504 which initiates the LID domain) are also protected upon fructose phosphate binding. These loops are probably indirectly stabilized through the contacts of the LID domain to the fructose.

We claim:

1. A crystal of
(a) a glucokinase regulatory protein (GKRP) comprising
(i) at least 82% identity to SEQ ID NO: 2,
(ii) at least 82% identity to SEQ ID NO: 4,
(iii) at least 82% identity to SEQ ID NO: 6, or
(iv) at least 82% identity to SEQ ID NOS: 2 and 4, to SEQ ID NOS: 4 and 6, or to SEQ ID NOS: 2 and 6; or
(b) a deletion mutant (truncated form of GKRP) comprising
(i) at least 82% identity to positions 6 to 606 of SEQ ID NO: 2,
(ii) at least 82% identity to positions 6 to 606 of SEQ ID NO: 4,
(iii) at least 82% identity to positions 6 to 606 of SEQ ID NO: 6, or
(iv) at least 82% identity to positions 6 to 606 of SEQ ID NOS: 2 and 4, to positions 6 to 606 of SEQ ID NOS: 4 and 6, or to positions 6 to 606 of SEQ ID NOS: 2 and 6.

2. The crystal according to claim 1(a), wherein the GKRP comprises
(i) at least 85, 90, 95, 97.5, 98, 99 or 100% identity to SEQ ID NO: 2,
(ii) at least 85, 90, 95, 97.5, 98, 99 or 100% identity to SEQ ID NO: 4, or
(iii) at least 85, 90, 95, 97.5, 98, 99 or 100% identity to SEQ ID NO: 6; or
according to claim 1(b), wherein the deletion mutant (truncated form) of GKRP comprises
(i) at least 85, 90, 95, 97.5, 98, 99 or 100% identity to positions 6 to 606 of SEQ ID NO: 2,
(ii) at least 85, 90, 95, 97.5, 98, 99 or 100% identity to positions 6 to 606 of SEQ ID NO: 4, or
(iii) at least 85, 90, 95, 97.5, 98, 99 or 100% identity to positions 6 to 606 of SEQ ID NO: 6.

3. The crystal according to claim 1, wherein the GKRP or the deletion mutant (truncated form) of GKRP comprises point mutations selected from 1 to 20 additional amino acids that are added to the C- and/or N-terminus as tags.

4. The crystal according to claim 1, wherein the GKRP or the deletion mutant (truncated form) of GKRP comprises deletions of 1 to 50 amino acids from either the N-terminus (N-terminal truncation), C-terminus (C-terminal truncation) or both of the non-tagged GKRP or of the deletion mutant (truncated form) of GKRP.

5. The crystal according to claim 4, wherein the deletion is in the N-terminal 44 amino acids in the numbering according to SEQ ID NO: 2, the C-terminal 20 amino acids or both in the numbering according to SEQ ID NO: 2.

6. The crystal according to claim 1, wherein the GKRP or the deletion mutant (truncated form) of GKRP comprises point mutations selected from 1 to 15 deletions or substitutions of solvent exposed aminoacids.

7. The crystal according to claim 1, wherein the GKRP or the deletion mutant (truncated form) of GKRP is selected from hGKRP (SEQ ID NO: 2), mGKRP (SEQ ID NO: 4), rGKRP (SEQ ID NO: 6), hGKRP_C-His (SEQ ID NO: 8), hGKRP_C-His_K326T/K327T (SEQ ID NO: 10), mGKRP_C-His (SEQ ID NO: 12) or rGKRP_C-His (SEQ ID NO: 14).

8. The crystal according to one claim 1, wherein the GKRP or the deletion mutant (truncated form) of GKRP is complexed with a low molecular weight binding ligand in the active site, wherein the low molecular weight binding ligand is selected from Fructose-1-Phosphate (F1P), Fructose-6-Phosphate (F6P), Orthophosphate (Pᵢ) or Sorbitol-6-Phosphate (S6P).

9. The crystal according to claim 1, wherein the GKRP or the deletion mutant (truncated form) of GKRP is hGKRP_C-His_K326T/K327T (SEQ ID NO. 10), and the low molecular weight binding ligand in the active site is selected from Fructose-1-Phosphate (F1P) or Orthophosphate (Pᵢ).

10. The crystal according to claim 1, wherein the GKRP or the deletion mutant (truncated form) of GKRP is complexed with one or more molecules of water, one or more cations, or both.

11. The crystal according to claim 1, wherein the GKRP or the deletion mutant (truncated form) of GKRP comprises a fructose-phosphate binding site at the interface between a SIS domain and a 2^{nd} α-helical domain with ubiquitin-like fold.

12. The crystal according to claim 1 having a space group P2₁2₁2₁.

13. The crystal according to claim 1 having a unit cell dimension between 60.0 and 62.0 Å for a, between 71.5 to 73.5 Å for b, and between 136.0 and 139.0 Å for c.

14. The crystal according to one claim 1, with amino acids having coordinates as shown in FIG. 2 or FIG. 3.

15. A polynucleotide encoding for a GKRP variant with at least one nucleotide different from SEQ ID NO: 1, 3 or 5 (other than wild type) as defined in claim 1.

16. The polynucleotide according to claim 15 encoding for a GKRP variant selected from SEQ ID NO: 8, 10, 12 or 14; or the polynucleotide of SEQ ID NO: 7, 9, 11, 13 or 15.

17. A vector comprising a polynucleotide encoding for a GKRP or GKRP variant according to claim 15.

18. A host cell comprising a polynucleotide encoding for a GKRP or GKRP variant according to claim 15.

19. A process for the crystallization of a GKRP or GKRP variant comprising the steps of:
(1) purification of the protein, and
(2) crystallization of the purified protein,
wherein step (2), the purified protein is complexed with a low molecular weight binding ligand in the active site.

20. A crystal of a GKRP or GKRP variant made by the process according to claim 19.


SEQUENCE LISTING (18 sequences)

SEQ ID NO: 1 - DNA, 1878 bp, Homo sapiens
  CDS (1)..(1875)
atgccaggcacaaaacggtttcaacatgtcattgagaccccggagcctggcaagtgggagttgtctgggtacgaggcagctgtgccaatcacggagaagtcaaacccactgacccaggatctagacaaagcagatgctgagaacattgttcgactgctagggcaatgtgatgctgagatcttccaggaggaggggcaagccctgtccacataccagagactctacagcgaatccattctgaccaccatggtacaggtggctgggaaagttcaggaagtgctgaaggagccagatggggggctggttgtgctgagtggagggggcacctctggccggatggcattcctcatgtcggtgtcctttaatcagctgatgaaaggtctgggacagaaacctctttacacctacctcattgcaggtggtgacaggtctgtggtggcctctagggaggggacagaagatagtgccttgcacgggattgaggaactgaagaaggtggctgccgggaagaagagagtgattgtcattggcatttctgtgggactctctgctccctttgtggcaggccagatggactgctgcatgaacaacacagctgtcttcttgccagtcctggttggcttcaatccagtgagcatggccagaaatgaccccattgaagactggagttcaacattccgacaagtagcagagcggatgcagaaaatgcaggagaaacagaaagcttttgtgctcaatcctgccatcgggcccgagggtctcagcggctcctcccggatgaaaggtggaagtgccaccaagattctgctggaaaccctgttattagcagcccataagactgtggaccagggcattgcagcatctcaaagatgcctcctggaaatcttgcggacatttgagcgagctcatcaggtgacctacagccaaagccccaagattgccaccctgatgaagagtgtcagcaccagtctggagaagaaaggccacgtgtacctggttggctggcagaccctgggcatcattgccatcatggatggagtagagtgcatccacacctttggtgctgatttccgagatgtccgtggctttctcattggtgatcacagtgacatgtttaaccagaaggctgagctcaccaaccagggtccccagttcaccttctcccaggaggacttcctgacttccatccttccctctctcacggaaatcgatactgtggtcttcattttcaccctggatgacaacctcacggaggtgcagactatagtggagcaggtgaaagagaagaccaaccacatccaggccctggcacacagcaccgtgggtcagaccttgctgatccctctgaagaagctctttccctccatcatcagcatcacatggccactgcttttctttgaatatgaagggaacttcatccagaagttccagcgtgagctaagcaccaaatgggtgctgaatacagtgagtacaggtgctcatgtgcttcttggtaagatcctacaaaaccacatgttggaccttcggattagcaactccaagctcttctggcgggcgctggccatgctgcagcggttctctggacagtccaaggctcgatgcatcgagagcctcctccgagcgatccactttccccagccactgtcagatgatattcgggctgctcccatctcctgccatgtccaggttgcacatgagaaggaacaggtgatacccatcgccttgctgagcctcctattccggtgctcgatcactgaggctcaggcacacctggctgcagctccttctgtctgtgaggctgtcaggagtgctcttgctgggccaggtcagaagcgcactgcggaccccctcgagatcctagagcctgacgttcagtga

SEQ ID NO: 2 - protein, 625 aa, Homo sapiens
MPGTKRFQHVIETPEPGKWELSGYEAAVPITEKSNPLTQDLDKADAENIVRLLGQCDAEIFQEEGQALSTYQRLYSESILTTMVQVAGKVQEVLKEPDGGLVVLSGGGTSGRMAFLMSVSFNQLMKGLGQKPLYTYLIAGGDRSVVASREGTEDSALHGIEELKKVAAGKKRVIVIGISVGLSAPFVAGQMDCCMNNTAVFLPVLVGFNPVSMARNDPIEDWSSTFRQVAERMQKMQEKQKAFVLNPAIGPEGLSGSSRMKGGSATKILLETLLLAAHKTVDQGIAASQRCLLEILRTFERAHQVTYSQSPKIATLMKSVSTSLEKKGHVYLVGWQTLGIIAIMDGVECIHTFGADFRDVRGFLIGDHSDMFNQKAELTNQGPQFTFSQEDFLTSILPSLTEIDTVVFIFTLDDNLTEVQTIVEQVKEKTNHIQALAHSTVGQTLLIPLKKLFPSIISITWPLLFFEYEGNFIQKFQRELSTKWVLNTVSTGAHVLLGKILQNHMLDLRISNSKLFWRALAMLQRFSGQSKARCIESLLRAIHFPQPLSDDIRAAPISCHVQVAHEKEQVIPIALLSLLFRCSITEAQAHLAAAPSVCEAVRSALAGPGQKRTADPLEILEPDVQ

SEQ ID NO: 3 - DNA, 1764 bp, Mus musculus
  CDS (1)..(1764)
atgccaagcaccaagcggtatcagcatgtgatcgagacccctgagcctggggaatgggagttgtcagggtatgaagcagctgtgccaatcacagagaagtccaacccactgacccggaacttggacaaagcagatgcagagaaaattgttcaactgctggggcagtgtgatgctgagatattccaggaggaggggcaaatcatgcccacctaccagcgactgtacagtgagtcagttctgaccaccatgttgcaagtggctggcaaggtccaggaagtgctgaaggagccagatgggggcctggtggtgctgagtggagggggcacctctggtcgtatggcattccttatgtctgtgtctttcaaccagctgatgaaaggtctgggacaaaaacctctttacacatacctcattgcagggggtgacaggtctgttgtagcctctcgggaacggacagaagatagcgccctacacggaatcgaggagctgaagaaggtggctgctgggaaaaagagagtggtcgttataggcatttccgtgggactctctgcgccctttgtggcaggccagatggactactgcatggataacacagctgtcttcttgccggtcctggttggcttcaatccggtgagcatggccagaaatgatcccattgaagactggagatcgacattccgacaagtggcagagcggatgcagaagatgcaggagaaacaggaagcctttgtgctcaatcctgccatcgggcctgaggggctcagtggctcttcccgaatgaaaggtggaagcgccaccaagattctactggaaaccctgctactagcagcccataagactgtggaccagggtgttgtgtcctctcaaagatgccttctggaaatcctgaggacatttgagcgggctcatcaggtaacctacagtcaaagttccaaaattgccactctgacgaagcaagttggcatcagcctggagaaaaaaggccacgtgcacttggttggctggcagaccctcggtatcatcgccattatggatggggtagagtgtatccacacttttggtgctgatttccgagatatccgtggctttcttattggtgaccacaatgacatgtttaaccagaaggatgagctcagcaatcagggtccccagttcaccttctctcaggatgacttcctgacttctgttctgccatcccttacggaaattgacactgtggtcttcatttttaccctggatgataacctcgcagaagtacaggccctggcagaaagggtgagggagaagagttggaacatccaggccctggtgcacagcacagtggggcagtccttgccagctcctctaaagaagctctttccctcgctcatcagcatcacatggccacttcttttcttcgattatgaagggagctacgttcagaagttccagcgtgagttaagcaccaagtgggtgttgaatacaaggttctcaggacagtccaaggctcgctgcattgagagtcttcttcaagtgatacatttccctcaaccgctgtcgaatgatgtccgcgcggcccccatctcctgccatgtccaggttgcccacgagaaggaaaaggtgatccccacagccttgctgagtctcctactcaggtgctccatcactgaggctaaggaacgcctggctgcagcttcttcagtctgtgaggttgttaggagcgccctctctgggccaggtcagaaacgcagcatccaagcctttggagaccctgtggtgccctga

SEQ ID NO: 4 - protein, 587 aa, Mus musculus
MPSTKRYQHVIETPEPGEWELSGYEAAVPITEKSNPLTRNLDKADAEKIVQLLGQCDAEIFQEEGQIMPTYQRLYSESVLTTMLQVAGKVQEVLKEPDGGLVVLSGGGTSGRMAFLMSVSFNQLMKGLGQKPLYTYLIAGGDRSVVASRERTEDSALHGIEELKKVAAGKKRVVVIGISVGLSAPFVAGQMDYCMDNTAVFLPVLVGFNPVSMARNDPIEDWRSTFRQVAERMQKMQEKQEAFVLNPAIGPEGLSGSSRMKGGSATKILLETLLLAAHKTVDQGVVSSQRCLLEILRTFERAHQVTYSQSSKIATLTKQVGISLEKKGHVHLVGWQTLGIIAIMDGVECIHTFGADFRDIRGFLIGDHNDMFNQKDELSNQGPQFTFSQDDFLTSVLPSLTEIDTVVFIFTLDDNLAEVQALAERVREKSWNIQALVHSTVGQSLPAPLKKLFPSLISITWPLLFFDYEGSYVQKFQRELSTKWVLNTRFSGQSKARCIESLLQVIHFPQPLSNDVRAAPISCHVQVAHEKEKVIPTALLSLLLRCSITEAKERLAAASSVCEVVRSALSGPGQKRSIQAFGDPVVP

SEQ ID NO: 5 - DNA, 1884 bp, Rattus norvegicus
  CDS (1)..(1884)
atgccaggcaccaaacgatatcagcatgtgatcgagacccctgagcctggtgaatgggagttgtcagggtatgaagcggctgtgccaatcacagagaaatccaacccactgacccgaaacctggacaaagcagatgcagagaaaattgtcaaactgctggggcagtgtgatgctgagatattccaggaggaggggcagattgtgcccacctaccagcgactatacagcgaatcagttctgaccaccatgttgcaagtggctggaaaagtccaggaagttctgaaggagccagatgggggtctggtagtgctgagtggagggggaacctctggtcgtatggcatttctcatgtctgtgtctttcaaccagctgatgaaaggcctgggacaaaagcctctttacacctacctcattgcaggaggtgacaggtctgttgtggcctctcgtgaacagacagaagatagcgccctacacgggatcgaggagctgaagaaggtggctgctgggaagaagagagtggtcgtcataggcatctctgtgggactctctgcgccctttgtggcaggtcagatggactactgcatggataacacagccgtcttcttgccggttctggttggcttcaatccagtgagcatggccagaaatgaccccattgaagactggagatcaacattccggcaagtggcagagcggatgcaaaagatgcaggagaaacaggaagcttttgtgctcaatcctgccatcgggcccgaggggctcagcggctcttcccgaatgaaaggtggaggtgccaccaagattctactggaaaccctgctactagcagcccataagactgtggaccagggtgttgtgtcctctcaaagatgccttctggaaatcctgaggacatttgagcgggctcatcaggtgacctacagtcaaagttccaaaattgccacgctgatgaaacaagtcggcatcagcctggagaagaaaggccgagtgcacttggttggctggcagactctcggcatcattgccattatggacggagtagagtgcatccacacttttggtgctgatttccaagatatccgtggctttcttattggtgaccacagtgacatgtttaaccagaaggatgaactcaccaaccagggtccccagttcaccttctcccaggatgacttcctgacttccatcctgccatccctcacggagactgacaccgtggtcttcatttttaccctggatgataacctcacagaagtacaggccctggcagaaagagtgagagagaagtgccagaacatccaggccctggtgcacagcactgtggggcagtccttgccggcccctctaaagaaactctttccctcactcatcagtatcacgtggccacttcttttcttcgattatgaagggacctatgttcagaagttccagcgtgagttaagcaccaagtgggtgttgaatacagtgagtactggggcccatgtactgctggggaagatcctacagaaccacatgctggacctccgcatcgccaactccaagctcttctggagggcgctggccatgttgcagaggttctctggacagtccaaggctcgctgcattgagagcctccttcaagcaatccactttcctcaaccactgtcggatgatgtccgcgccgctcccatctcctgccacgtccaggttgcccacgagaaggaaaaggtgatccccacagccttgctgagcctcctactccggtgctccatctctgaggctaaggcacgcctgtctgcagcttcttcagtctgtgaggttgttaggagcgccctctctgggccgggtcagaagcgcagcacgcaagcccttgaagaccctcccgcctgtgggaccctgaattga

SEQ ID NO: 6 - protein, 627 aa, Rattus norvegicus [sequence omitted]

SEQ ID NO: 7 - DNA, 1905 bp, artificial
  human GKRP comprising C-terminal His-tag; codon optimized
atgcccggcaccaagcgtttccagcacgtgatcgagactcccgagcccggcaagtgggagctgtccggttacgaggctgctgtgcccatcaccgagaagtccaaccccctgacccaggacctggacaaggctgacgctgagaacatcgtgcgtctgctgggccagtgcgacgctgagatcttccaggaagaaggccaggctctgtccacctaccagcgcctgtactccgagtccatcctgaccactatggtgcaagtggccggcaaggtgcaggaagtgctgaaggaacccgacggcggtctggtggtgctgtctggtggcggcacctccggtcgtatggctttcctgatgtccgtgtccttcaaccagctgatgaagggtctgggccagaagcccctgtacacctacctgatcgctggcggtgaccgttccgtcgtcgcttcccgtgagggcaccgaggactccgctctgcacggtatcgaggaactgaagaaggtggccgctggcaagaagcgtgtcatcgtcatcggtatctccgtgggcctgtccgctcccttcgtggctggccagatggactgctgcatgaacaacaccgctgtgttcctccccgtgctggtcggtttcaaccccgtgtccatggctcgtaacgaccccatcgaggactggtcctccaccttccgtcaggtggccgagcgtatgcagaagatgcaggaaaagcagaaggctttcgtcctgaaccccgctatcggtcccgagggactgtctggttcctcccgtatgaagggcggttccgctaccaagatcctgctcgagactctgctgctggctgctcacaagaccgtggaccagggtatcgctgcttcccagcgttgcctcctcgagatcctgcgtaccttcgagcgtgctcaccaggtgacctactcccagtcccccaagatcgctaccctgatgaagtccgtgtccacctccctcgagaagaagggtcacgtctacctggtcggttggcagaccctgggtatcatcgctatcatggacggtgtcgagtgcatccacaccttcggtgctgacttccgtgacgtgcgcggtttcctgatcggtgaccactccgacatgttcaaccagaaggccgagctgaccaaccagggtccccagttcaccttctcccaggaagatttcctgacctccatcctgccctccctgaccgagatcgacaccgtggtgttcatcttcaccctggacgacaacctgaccgaggtgcagaccatcgtggagcaggtcaaagaaaagaccaaccacatccaggctctggctcactccaccgtcggccagaccctgcccatccccctgaagaagctgttcccctccatcatctccatcacctggcccctgctgttcttcgagtacgagggcaacttcatccagaagttccagcgcgagctgtccaccaagtgggtgctgaacaccgtgtctaccggtgctcacgtgctgctgggaaagatcctgcagaaccacatgctggacctgcgtatctccaactccaagctgttctggcgtgctctggctatgctgcagcgtttctccggccagtccaaggctcgttgcatcgagtccctgctgcgtgctatccacttcccccagcccctgtccgacgacatccgtgctgctcccatctcctgccacgtgcaggtcgcccacgagaaggaacaggtcatccctatcgctctgctgtccctgctcttccgttgctctatcaccgaggctcaggctcacctggctgctgctccctccgtgtgcgaggctgtgcgttccgctctggctggtcccggccagaagcgtaccgctgaccctctcgagatcctcgagcccgacgtgcagctcgagcaccaccaccatcatcactaatga

SEQ ID NO: 8 - protein, 633 aa, artificial
  Synthetic Construct
MPGTKRFQHVIETPEPGKWELSGYEAAVPITEKSNPLTQDLDKADAENIVRLLGQCDAEIFQEEGQALSTYQRLYSESILTTMVQVAGKVQEVLKEPDGGLVVLSGGGTSGRMAFLMSVSFNQLMKGLGQKPLYTYLIAGGDRSVVASREGTEDSALHGIEELKKVAAGKKRVIVIGISVGLSAPFVAGQMDCCMNNTAVFLPVLVGFNPVSMARNDPIEDWSSTFRQVAERMQKMQEKQKAFVLNPAIGPEGLSGSSRMKGGSATKILLETLLLAAHKTVDQGIAASQRCLLEILRTFERAHQVTYSQSPKIATLMKSVSTSLEKKGHVYLVGWQTLGIIAIMDGVECIHTFGADFRDVRGFLIGDHSDMFNQKAELTNQGPQFTFSQEDFLTSILPSLTEIDTVVFIFTLDDNLTEVQTIVEQVKEKTNHIQALAHSTVGQTLPIPLKKLFPSIISITWPLLFFEYEGNFIQKFQRELSTKWVLNTVSTGAHVLLGKILQNHMLDLRISNSKLFWRALAMLQRFSGQSKARCIESLLRAIHFPQPLSDDIRAAPISCHVQVAHEKEQVIPIALLSLLFRCSITEAQAHLAAAPSVCEAVRSALAGPGQKRTADPLEILEPDVQLEHHHHHH

SEQ ID NO: 9 - DNA, 1905 bp, artificial
  human GKRP comprising C-terminal His-tag; codon optimized; variant K326T/K327T
atgcccggcaccaagcgtttccagcacgtgatcgagactcccgagcccggcaagtgggagctgtccggttacgaggctgctgtgcccatcaccgagaagtccaaccccctgacccaggacctggacaaggctgacgctgagaacatcgtgcgtctgctgggccagtgcgacgctgagatcttccaggaagaaggccaggctctgtccacctaccagcgcctgtactccgagtccatcctgaccactatggtgcaagtggccggcaaggtgcaggaagtgctgaaggaacccgacggcggtctggtggtgctgtctggtggcggcacctccggtcgtatggctttcctgatgtccgtgtccttcaaccagctgatgaagggtctgggccagaagcccctgtacacctacctgatcgctggcggtgaccgttccgtcgtcgcttcccgtgagggcaccgaggactccgctctgcacggtatcgaggaactgaagaaggtggccgctggcaagaagcgtgtcatcgtcatcggtatctccgtgggcctgtccgctcccttcgtggctggccagatggactgctgcatgaacaacaccgctgtgttcctccccgtgctggtcggtttcaaccccgtgtccatggctcgtaacgaccccatcgaggactggtcctccaccttccgtcaggtggccgagcgtatgcagaagatgcaggaaaagcagaaggctttcgtcctgaaccccgctatcggtcccgagggactgtctggttcctcccgtatgaagggcggttccgctaccaagatcctgctcgagactctgctgctggctgctcacaagaccgtggaccagggtatcgctgcttcccagcgttgcctcctcgagatcctgcgtaccttcgagcgtgctcaccaggtgacctactcccagtcccccaagatcgctaccctgatgaagtccgtgtccacctccctcgagaccaccggtcacgtctacctggtcggttggcagaccctgggtatcatcgctatcatggacggtgtcgagtgcatccacaccttcggtgctgacttccgtgacgtgcgcggtttcctgatcggtgaccactccgacatgttcaaccagaaggccgagctgaccaaccagggtccccagttcaccttctcccaggaagatttcctgacctccatcctgccctccctgaccgagatcgacaccgtggtgttcatcttcaccctggacgacaacctgaccgaggtgcagaccatcgtggagcaggtcaaagaaaagaccaaccacatccaggctctggctcactccaccgtcggccagaccctgcccatccccctgaagaagctgttcccctccatcatctccatcacctggcccctgctgttcttcgagtacgagggcaacttcatccagaagttccagcgcgagctgtccaccaagtgggtgctgaacaccgtgtctaccggtgctcacgtgctgctgggaaagatcctgcagaaccacatgctggacctgcgtatctccaactccaagctgttctggcgtgctctggctatgctgcagcgtttctccggccagtccaaggctcgttgcatcgagtccctgctgcgtgctatccacttcccccagcccctgtccgacgacatccgtgctgctcccatctcctgccacgtgcaggtcgcccacgagaaggaacaggtcatccctatcgctctgctgtccctgctcttccgttgctctatcaccgaggctcaggctcacctggctgctgctccctccgtgtgcgaggctgtgcgttccgctctggctggtcccggccagaagcgtaccgctgaccctctcgagatcctcgagcccgacgtgcagctcgagcaccaccaccatcatcactaatga

SEQ ID NO: 10 - protein, 633 aa, artificial
  Synthetic Construct
MPGTKRFQHVIETPEPGKWELSGYEAAVPITEKSNPLTQDLDKADAENIVRLLGQCDAEIFQEEGQALSTYQRLYSESILTTMVQVAGKVQEVLKEPDGGLVVLSGGGTSGRMAFLMSVSFNQLMKGLGQKPLYTYLIAGGDRSVVASREGTEDSALHGIEELKKVAAGKKRVIVIGISVGLSAPFVAGQMDCCMNNTAVFLPVLVGFNPVSMARNDPIEDWSSTFRQVAERMQKMQEKQKAFVLNPAIGPEGLSGSSRMKGGSATKILLETLLLAAHKTVDQGIAASQRCLLEILRTFERAHQVTYSQSPKIATLMKSVSTSLETTGHVYLVGWQTLGIIAIMDGVECIHTFGADFRDVRGFLIGDHSDMFNQKAELTNQGPQFTFSQEDFLTSILPSLTEIDTVVFIFTLDDNLTEVQTIVEQVKEKTNHIQALAHSTVGQTLPIPLKKLFPSIISITWPLLFFEYEGNFIQKFQRELSTKWVLNTVSTGAHVLLGKILQNHMLDLRISNSKLFWRALAMLQRFSGQSKARCIESLLRAIHFPQPLSDDIRAAPISCHVQVAHEKEQVIPIALLSLLFRCSITEAQAHLAAAPSVCEAVRSALAGPGQKRTADPLEILEPDVQLEHHHHHH

SEQ ID NO: 11 - DNA, 1896 bp, artificial
  mouse GKRP comprising C-terminal His-tag
atgccaagcaccaagcggtatcagcatgtgatcgagacccctgagcctggggaatgggagttgtcagggtatgaagcagctgtgccaatcacagagaagtccaacccactgacccggaacttggacaaagcagatgcagagaaaattgttcaactgctggggcagtgtgatgctgagatattccaggaggaggggcaaatcatgcccacctaccagcgactgtacagtgagtcagttctgaccaccatgttgcaagtggctggcaaggtccaggaagtgctgaaggagccagatgggggcctggtggtgctgagtggagggggcacctctggtcgtatggcattccttatgtctgtgtctttcaaccagctgatgaaaggtctgggacaaaaacctctttacacatacctcattgcagggggtgacaggtctgttgtagcctctcgggaacggacagaagatagcgccctacacggaatcgaggagctgaagaaggtggctgctgggaaaaagagagtggtcgttataggcatttccgtgggactctctgcgccctttgtggcaggccagatggactactgcatggataacacagctgtcttcttgccggtcctggttggcttcaatccggtgagcatggccagaaatgatcccattgaagactggagatcgacattccgacaagtggcagagcggatgcagaagatgcaggagaaacaggaagcctttgtgctcaatcctgccatcgggcctgaggggctcagtggctcttcccgaatgaaaggtggaagcgccaccaagattctactggaaaccctgctactagcagcccataagactgtggaccagggtgttgtgtcctctcaaagatgccttctggaaatcctgaggacatttgagcgggctcatcaggtaacctacagtcaaagttccaaaattgccactctgacgaagcaagttggcatcagcctggagaaaaaaggccacgtgcacttggttggctggcagaccctcggtatcatcgccattatggatggggtagagtgtatccacacttttggtgctgatttccgagatatccgtggctttcttattggtgaccacaatgacatgtttaaccagaaggatgagctcagcaatcagggtccccagttcaccttctctcaggatgacttcctgacttctgttctgccatcccttacggaaattgacactgtggtcttcatttttaccctggatgataacctcgcagaagtacaggccctggcagaaagggtgagggagaagagttggaacatccaggccctggtgcacagcacagtggggcagtccttgccagctcctctaaagaagctctttccctcgctcatcagcatcacatggccacttcttttcttcgattatgaagggagctacgttcagaagttccagcgtgagttaagcaccaagtgggtgttgaatacagtgagtactggggcccatgtgctgctggggaagatcctacagaaccacatgctggacctccgcatcgccaactccaaactcttctggagggcactggccatgttgcagaggttctcaggacagtccaaggctcgctgcattgagagtcttcttcaagtgatacatttccctcaaccgctgtcgaatgatgtccgcgcggcccccatctcctgccatgtccaggttgcccacgagaaggaaaaggtgatccccacagccttgctgagtctcctactcaggtgctccatcactgaggctaaggaacgcctggctgcagcttcttcagtctgtgaggttgttaggagcgccctctctgggccaggtcagaaacgcagcatccaagcctttggagaccctgtggtgcccgtcgagcaccaccaccaccaccactaa

SEQ ID NO: 12 - protein, 631 aa, artificial
  Synthetic Construct
MPSTKRYQHVIETPEPGEWELSGYEAAVPITEKSNPLTRNLDKADAEKIVQLLGQCDAEIFQEEGQIMPTYQRLYSESVLTTMLQVAGKVQEVLKEPDGGLVVLSGGGTSGRMAFLMSVSFNQLMKGLGQKPLYTYLIAGGDRSVVASRERTEDSALHGIEELKKVAAGKKRVVVIGISVGLSAPFVAGQMDYCMDNTAVFLPVLVGFNPVSMARNDPIEDWRSTFRQVAERMQKMQEKQEAFVLNPAIGPEGLSGSSRMKGGSATKILLETLLLAAHKTVDQGVVSSQRCLLEILRTFERAHQVTYSQSSKIATLTKQVGISLEKKGHVHLVGWQTLGIIAIMDGVECIHTFGADFRDIRGFLIGDHNDMFNQKDELSNQGPQFTFSQDDFLTSVLPSLTEIDTVVFIFTLDDNLAEVQALAERVREKSWNIQALVHSTVGQSLPAPLKKLFPSLISITWPLLFFDYEGSYVQKFQRELSTKWVLNTVSTGAHVLLGKILQNHMLDLRIANSKLFWRALAMLQRFSGQSKARCIESLLQVIHFPQPLSNDVRAAPISCHVQVAHEKEKVIPTALLSLLLRCSITEAKERLAAASSVCEVVRSALSGPGQKRSIQAFGDPVVPVEHHHHHH

SEQ ID NO: 13 - DNA, 1929 bp, artificial
  rat GKRP comprising C-terminal His-tag
atgccaggcaccaaacgatatcagcatgtgatcgagacccctgagcctggtgaatgggagttgtcagggtatgaagcggctgtgccaatcacagagaaatccaacccactgacccgaaacctggacaaagcagatgcagagaaaattgtcaaactgctggggcagtgtgatgctgagatattccaggaggaggggcagattgtgcccacctaccagcgactatacagcgaatcagttctgaccaccatgttgcaagtggctggaaaagtccaggaagttctgaaggagccagatgggggtctggtagtgctgagtggagggggaacctctggtcgtatggcatttctcatgtctgtgtctttcaaccagctgatgaaaggcctgggacaaaagcctctttacacctacctcattgcaggaggtgacaggtctgttgtggcctctcgtgaacagacagaagatagcgccctacacgggatcgaggagctgaagaaggtggctgctgggaagaagagagtggtcgtcataggcatctctgtgggactctctgcgccctttgtggcaggtcagatggactactgcatggataacacagccgtcttcttgccggttctggttggcttcaatccagtgagcatggccagaaatgaccccattgaagactggagatcaacattccggcaagtggcagagcggatgcaaaagatgcaggagaaacaggaagcttttgtgctcaatcctgccatcgggcccgaggggctcagcggctcttcccgaatgaaaggtggaggtgccaccaagattctactggaaaccctgctactagcagcccataagactgtggaccagggtgttgtgtcctctcaaagatgccttctggaaatcctgaggacatttgagcgggctcatcaggtgacctacagtcaaagttccaaaattgccacgctgatgaaacaagtcggcatcagcctggagaagaaaggccgagtgcacttggttggctggcagactctcggcatcattgccattatggacggagtagagtgcatccacacttttggtgctgatttccaagatatccgtggctttcttattggtgaccacagtgacatgtttaaccagaaggatgaactcaccaaccagggtccccagttcaccttctcccaggatgacttcctgacttccatcctgccatccctcacggagactgacaccgtggtcttcatttttaccctggatgataacctcacagaagtacaggccctggcagaaagagtgagagagaagtgccagaacatccaggccctggtgcacagcactgtggggcagtccttgccggcccctctaaagaaactctttccctcactcatcagtatcacgtggccacttcttttcttcgattatgaagggacctatgttcagaagttccagcgtgagttaagcaccaagtgggtgttgaatacagtgagtactggggcccatgtactgctggggaagatcctacagaaccacatgctggacctccgcatcgccaactccaagctcttctggagggcgctggccatgttgcagaggttctctggacagtccaaggctcgctgcattgagagcctccttcaagcaatccactttcctcaaccactgtcggatgatgtccgcgccgctcccatctcctgccacgtccaggttgcccacgagaaggaaaaggtgatccccacagccttgctgagcctcctactccggtgctccatctctgaggctaaggcacgcctgtctgcagcttcttcagtctgtgaggttgttaggagcgccctctctgggccgggtcagaagcgcagcacgcaagcccttgaagaccctcccgcctgtgggaccctgaatgtcgacaagcttgcggccgcactcgagcaccaccaccaccaccactga

SEQ ID NO: 14 - protein, 642 aa, artificial
  Synthetic Construct
MPGTKRYQHVIETPEPGEWELSGYEAAVPITEKSNPLTRNLDKADAEKIVKLLGQCDAEIFQEEGQIVPTYQRLYSESVLTTMLQVAGKVQEVLKEPDGGLVVLSGGGTSGRMAFLMSVSFNQLMKGLGQKPLYTYLIAGGDRSVVASREQTEDSALHGIEELKKVAAGKKRVVVIGISVGLSAPFVAGQMDYCMDNTAVFLPVLVGFNPVSMARNDPIEDWRSTFRQVAERMQKMQEKQEAFVLNPAIGPEGLSGSSRMKGGGATKILLETLLLAAHKTVDQGVVSSQRCLLEILRTFERAHQVTYSQSSKIATLMKQVGISLEKKGRVHLVGWQTLGIIAIMDGVECIHTFGADFQDIRGFLIGDHSDMFNQKDELTNQGPQFTFSQDDFLTSILPSLTETDTVVFIFTLDDNLTEVQALAERVREKCQNIQALVHSTVGQSLPAPLKKLFPSLISITWPLLFFDYEGTYVQKFQRELSTKWVLNTVSTGAHVLLGKILQNHMLDLRIANSKLFWRALAMLQRFSGQSKARCIESLLQAIHFPQPLSDDVRAAPISCHVQVAHEKEKVIPTALLSLLLRCSISEAKARLSAASSVCEVVRSALSGPGQKRSTQALEDPPACGTLNVDKLAAALEHHHHHH

SEQ ID NO: 15 - DNA, 1878 bp, artificial
  human GKRP comprising no C-terminal His-tag; codon optimized
atgcccggcaccaagcgtttccagcacgtgatcgagactcccgagcccggcaagtgggagctgtccggttacgaggctgctgtgcccatcaccgagaagtccaaccccctgacccaggacctggacaaggctgacgctgagaacatcgtgcgtctgctgggccagtgcgacgctgagatcttccaggaagaaggccaggctctgtccacctaccagcgcctgtactccgagtccatcctgaccactatggtgcaagtggccggcaaggtgcaggaagtgctgaaggaacccgacggcggtctggtggtgctgtctggtggcggcacctccggtcgtatggctttcctgatgtccgtgtccttcaaccagctgatgaagggtctgggccagaagcccctgtacacctacctgatcgctggcggtgaccgttccgtcgtcgcttcccgtgagggcaccgaggactccgctctgcacggtatcgaggaactgaagaaggtggccgctggcaagaagcgtgtcatcgtcatcggtatctccgtgggcctgtccgctcccttcgtggctggccagatggactgctgcatgaacaacaccgctgtgttcctccccgtgctggtcggtttcaaccccgtgtccatggctcgtaacgaccccatcgaggactggtcctccaccttccgtcaggtggccgagcgtatgcagaagatgcaggaaaagcagaaggctttcgtcctgaaccccgctatcggtcccgagggactgtctggttcctcccgtatgaagggcggttccgctaccaagatcctgctcgagactctgctgctggctgctcacaagaccgtggaccagggtatcgctgcttcccagcgttgcctcctcgagatcctgcgtaccttcgagcgtgctcaccaggtgacctactcccagtcccccaagatcgctaccctgatgaagtccgtgtccacctccctcgagaagaagggtcacgtctacctggtcggttggcagaccctgggtatcatcgctatcatggacggtgtcgagtgcatccacaccttcggtgctgacttccgtgacgtgcgcggtttcctgatcggtgaccactccgacatgttcaaccagaaggccgagctgaccaaccagggtccccagttcaccttctcccaggaagatttcctgacctccatcctgccctccctgaccgagatcgacaccgtggtgttcatcttcaccctggacgacaacctgaccgaggtgcagaccatcgtggagcaggtcaaagaaaagaccaaccacatccaggctctggctcactccaccgtcggccagaccctgcccatccccctgaagaagctgttcccctccatcatctccatcacctggcccctgctgttcttcgagtacgagggcaacttcatccagaagttccagcgcgagctgtccaccaagtgggtgctgaacaccgtgtctaccggtgctcacgtgctgctgggaaagatcctgcagaaccacatgctggacctgcgtatctccaactccaagctgttctggcgtgctctggctatgctgcagcgtttctccggccagtccaaggctcgttgcatcgagtccctgctgcgtgctatccacttcccccagcccctgtccgacgacatccgtgctgctcccatctcctgccacgtgcaggtcgcccacgagaaggaacaggtcatccctatcgctctgctgtccctgctcttccgttgctctatcaccgaggctcaggctcacctggctgctgctccctccgtgtgcgaggctgtgcgttccgctctggctggtcccggccagaagcgtaccgctgaccctctcgagatcctcgagcccgacgtgcagtga

SEQ ID NO: 16 - protein, 625 aa, artificial [sequence omitted]

SEQ ID NO: 17 - DNA, 25 bp, artificial
  Primer attB1
acaagtttgtacaaaaaagcaggct

SEQ ID NO: 18 - DNA, 24 bp, artificial
  Primer attB2
accactttgtacaagaaagctggt